SEQUENCE LISTING (548 sequences)

SEQ ID NO: 1 - protein, 535 aa, Artificial Sequence
  source: /note=“Description of Artificial Sequence: Synthetic polypeptide”
MALPVTALLLPLALLLHAARPGSEIQLQQSGAELVKPGASVKLSCTGSGFNIEDYYIHWVKQRTEQGLEWIGRIDPENDETKYGPIFQGRATITADTSSNTVYLQLSSLTSEDTAVYYCAFRGGVYWGPGTTLTVSSGGGGSGGGGSGGGGSHMDVVMTQSPLTLSVAIGQSASISCKSSQSLLDSDGKTYLNWLLQRPGQSPKRLISLVSKLDSGVPDRFTGSGSGTDFTLRISRVEAEDLGIYYCWQGTHFPGTFGGGTKLEIKASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 491 aa, Artificial Sequence
  source: /note=“Description of Artificial Sequence: Synthetic polypeptide”
MALPVTALLLPLALLLHAARPGSEIQLQQSGAELVKPGASVKLSCTGSGFNIEDYYIHWVKQRTEQGLEWIGRIDPENDETKYGPIFQGRATITADTSSNTVYLQLSSLTSEDTAVYYCAFRGGVYWGPGTTLTVSSGGGGSGGGGSGGGGSHMDVVMTQSPLTLSVAIGQSASISCKSSQSLLDSDGKTYLNWLLQRPGQSPKRLISLVSKLDSGVPDRFTGSGSGTDFTLRISRVEAEDLGIYYCWQGTHFPGTFGGGTKLEIKASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 488 aa, Artificial Sequence
  source: /note=“Description of Artificial Sequence: Synthetic polypeptide”
MALPVTALLLPLALLLHAARPGSDIQMTQSPSSLSASVGDRVTITCRASQGIRNNLAWYQQKPGKAPKRLIYAASNLQSGVPSRFTGSGSGTEFTLIVSSLQPEDFATYYCLQHHSYPLTSGGGTKVEIKRTGSTSGSGKPGSGEGSEVQVLESGGGLVQPGGSLRLSCAASGFTFSSYAMSWVRQAPGKGLEWVSAISGSGGSTNYADSVKGRFTISRDNSKNTLYLQMNSLRAEDTAVYYCAGSSGWSEYWGQGTLVTVSSASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - DNA, 729 bp, Mus sp.
gagattcagctgcagcaatctggggcagaacttgtgaagccaggggcctcagtcaagctgtcctgcacaggttctggcttcaacattgaagactactatattcactgggtgaagcagaggactgaacagggcctggaatggattggaaggattgatcctgagaatgatgaaactaaatatggcccaatattccagggcagggccactataacagcagacacatcctccaacacagtctacctgcaactcagcagcctgacatctgaggacactgccgtctattactgtgcctttcgcggtggagtctactgggggccaggaaccactctcacagtctcctcaggaggtggtggttccggtggtggtggttccggaggtggtggttcacatatggatgttgtgatgacccagtctccactcactctatcggttgccattggacaatcagcctccatctcttgcaagtcaagtcagagcctcttagatagtgatggaaagacatatttgaattggttgttacagaggccaggccagtctccaaagcgcctaatctctctggtgtctaaactggactctggagtccctgacaggttcactggcagtggatcagggacagatttcacactgagaatcagcagagtggaggctgaggatttgggaatttattattgctggcaaggtacacattttcctgggacgttcggtggagggaccaagctggagataaaa

SEQ ID NO: 5 - DNA, 720 bp, Artificial Sequence
  source: /note=“Description of Artificial Sequence: Synthetic polynucleotide”
gacatccagatgacccagagccctagcagcctgagcgccagcgtgggcgacagagtgaccatcacctgtcgggccagccagggcatcagaaacaacctggcctggtatcagcagaagcccggcaaggcccccaagagactgatctacgctgccagcaatctgcagagcggcgtgcccagcagattcaccggaagcggctccggcaccgagttcaccctgatcgtgtccagcctgcagcccgaggacttcgccacctactactgcctgcagcaccacagctaccctctgaccagcggcggaggcaccaaggtggagatcaagcggaccggcagcaccagcggcagcggcaagcctggcagcggcgagggaagcgaggtccaggtgctggaatctggcggcggactggtgcagcctggcggcagcctgagactgagctgtgccgccagcggcttcaccttcagcagctacgccatgtcttgggtccggcaggctcctggaaagggcctggaatgggtgtccgccatcagcggctctggcggctccaccaactacgccgacagcgtgaagggccggttcaccatcagccgggacaacagcaagaacaccctgtatctgcagatgaacagcctgagagccgaggacaccgccgtgtactactgtgccggcagcagcgggtggagcgagtactggggccagggcacactggtcacagtgtctagc

SEQ ID NO: 6 - DNA, 63 bp, Artificial Sequence
  source: /note=“Description of Artificial Sequence: Synthetic oligonucleotide”
atggccttaccagtgaccgccttgctcctgccgctggccttgctgctccacgccgccaggccg

SEQ ID NO: 7 - DNA, 135 bp, Artificial Sequence
  source: /note=“Description of Artificial Sequence: Synthetic polynucleotide”
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 8 - DNA, 72 bp, Artificial Sequence
  source: /note=“Description of Artificial Sequence: Synthetic oligonucleotide”
atctacatctgggcgcccttggccgggacttgtggggtccttctcctgtcactggttatcaccctttactgc

SEQ ID NO: 9 - DNA, 126 bp, Artificial Sequence
  source: /note=“Description of Artificial Sequence: Synthetic polynucleotide”
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 10 - DNA, 336 bp, Artificial Sequence
  source: /note=“Description of Artificial Sequence: Synthetic polynucleotide”
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 11 - protein, 243 aa, Mus sp.
EIQLQQSGAELVKPGASVKLSCTGSGFNIEDYYIHWVKQRTEQGLEWIGRIDPENDETKYGPIFQGRATITADTSSNTVYLQLSSLTSEDTAVYYCAFRGGVYWGPGTTLTVSSGGGGSGGGGSGGGGSHMDVVMTQSPLTLSVAIGQSASISCKSSQSLLDSDGKTYLNWLLQRPGQSPKRLISLVSKLDSGVPDRFTGSGSGTDFTLRISRVEAEDLGIYYCWQGTHFPGTFGGGTKLEIK

SEQ ID NO: 12 - protein, 240 aa, Homo sapiens
DIQMTQSPSSLSASVGDRVTITCRASQGIRNNLAWYQQKPGKAPKRLIYAASNLQSGVPSRFTGSGSGTEFTLIVSSLQPEDFATYYCLQHHSYPLTSGGGTKVEIKRTGSTSGSGKPGSGEGSEVQVLESGGGLVQPGGSLRLSCAASGFTFSSYAMSWVRQAPGKGLEWVSAISGSGGSTNYADSVKGRFTISRDNSKNTLYLQMNSLRAEDTAVYYCAGSSGWSEYWGQGTLVTVSS

SEQ ID NO: 13 - protein, 21 aa, Artificial Sequence
  source: /note=“Description of Artificial Sequence: Synthetic peptide”
MALPVTALLLPLALLLHAARP

SEQ ID NO: 14 - protein, 45 aa, Artificial Sequence
  source: /note=“Description of Artificial Sequence: Synthetic polypeptide”
TTTPAPRPPTPAPTIASQPLSLRPEACRPAAGGAVHTRGLDFACD

SEQ ID NO: 15 - protein, 24 aa, Artificial Sequence
  source: /note=“Description of Artificial Sequence: Synthetic peptide”
IYIWAPLAGTCGVLLLSLVITLYC

SEQ ID NO: 16 - protein, 42 aa, Artificial Sequence
  source: /note=“Description of Artificial Sequence: Synthetic polypeptide”
KRGRKKLLYIFKQPFMRPVQTTQEEDGCSCRFPEEEEGGCEL

SEQ ID NO: 17 - protein, 112 aa, Artificial Sequence
  source: /note=“Description of Artificial Sequence: Synthetic polypeptide”
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - DNA, 1605 bp, Artificial Sequence
  source: /note=“Description of Artificial Sequence: Synthetic polynucleotide”
atggccttaccagtgaccgccttgctcctgccgctggccttgctgctccacgccgccaggccgggatccgagattcagctgcagcaatctggggcagaacttgtgaagccaggggcctcagtcaagctgtcctgcacaggttctggcttcaacattgaagactactatattcactgggtgaagcagaggactgaacagggcctggaatggattggaaggattgatcctgagaatgatgaaactaaatatggcccaatattccagggcagggccactataacagcagacacatcctccaacacagtctacctgcaactcagcagcctgacatctgaggacactgccgtctattactgtgcctttcgcggtggagtctactgggggccaggaaccactctcacagtctcctcaggaggtggtggttccggtggtggtggttccggaggtggtggttcacatatggatgttgtgatgacccagtctccactcactctatcggttgccattggacaatcagcctccatctcttgcaagtcaagtcagagcctcttagatagtgatggaaagacatatttgaattggttgttacagaggccaggccagtctccaaagcgcctaatctctctggtgtctaaactggactctggagtccctgacaggttcactggcagtggatcagggacagatttcacactgagaatcagcagagtggaggctgaggatttgggaatttattattgctggcaaggtacacattttcctgggacgttcggtggagggaccaagctggagataaaagctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 19 - DNA, 1473 bp, Artificial Sequence
  source: /note=“Description of Artificial Sequence: Synthetic polynucleotide”
atggccttaccagtgaccgccttgctcctgccgctggccttgctgctccacgccgccaggccgggatccgagattcagctgcagcaatctggggcagaacttgtgaagccaggggcctcagtcaagctgtcctgcacaggttctggcttcaacattgaagactactatattcactgggtgaagcagaggactgaacagggcctggaatggattggaaggattgatcctgagaatgatgaaactaaatatggcccaatattccagggcagggccactataacagcagacacatcctccaacacagtctacctgcaactcagcagcctgacatctgaggacactgccgtctattactgtgcctttcgcggtggagtctactgggggccaggaaccactctcacagtctcctcaggaggtggtggttccggtggtggtggttccggaggtggtggttcacatatggatgttgtgatgacccagtctccactcactctatcggttgccattggacaatcagcctccatctcttgcaagtcaagtcagagcctcttagatagtgatggaaagacatatttgaattggttgttacagaggccaggccagtctccaaagcgcctaatctctctggtgtctaaactggactctggagtccctgacaggttcactggcagtggatcagggacagatttcacactgagaatcagcagagtggaggctgaggatttgggaatttattattgctggcaaggtacacattttcctgggacgttcggtggagggaccaagctggagataaaagctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 20 - DNA, 1465 bp, Artificial Sequence
  source: /note=“Description of Artificial Sequence: Synthetic polynucleotide”
atggccttaccagtgaccgccttgctcctgccgctggccttgctgctccacgccgccaggccgggatccgacatccagatgacccagagccctagcagcctgagcgccagcgtgggcgacagagtgaccatcacctgtcgggccagccagggcatcagaaacaacctggcctggtatcagcagaagcccggcaaggcccccaagagactgatctacgctgccagcaatctgcagagcggcgtgcccagcagattcaccggaagcggctccggcaccgagttcaccctgatcgtgtccagcctgcagcccgaggacttcgccacctactactgcctgcagcaccacagctaccctctgaccagcggcggaggcaccaaggtggagatcaagcggaccggcagcaccagcggcagcggcaagcctggcagcggcgagggaagcgaggtccaggtgctggaatctggcggcggactggtgcagcctggcggcagcctgagactgagctgtgccgccagcggcttcaccttcagcagctacgccatgtcttgggtccggcaggctcctggaaagggcctggaatgggtgtccgccatcagcggctctggcggctccaccaactacgccgacagcgtgaagggccggttcaccatcagccgggacaacagcaagaacaccctgtatctgcagatgaacagcctgagagccgaggacaccgccgtgtactactgtgccggcagcagcgggtggagcgagtactggggccagggcacactggtcacagtgtctagcgctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgct

SEQ ID NO: 21 - protein, 114 aa, Artificial Sequence
  source: /note=“Description of Artificial Sequence: Synthetic polypeptide”
EIQLQQSGAELVKPGASVKLSCTGSGFNIEDYYIHWVKQRTEQGLEWIGRIDPENDETKYGPIFQGRATITADTSSNTVYLQLSSLTSEDTAVYYCAFRGGVYWGPGTTLTVSS

SEQ ID NO: 22 - protein, 5 aa, Artificial Sequence
  source: /note=“Description of Artificial Sequence: Synthetic peptide”
DYYIH

SEQ ID NO: 23 - protein, 17 aa, Artificial Sequence
  source: /note=“Description of Artificial Sequence: Synthetic peptide”
RIDPENDETKYGPIFQG

SEQ ID NO: 24 - protein, 5 aa, Artificial Sequence
  source: /note=“Description of Artificial Sequence: Synthetic peptide”
RGGVY

SEQ ID NO: 25 - protein, 112 aa, Artificial Sequence
  source: /note=“Description of Artificial Sequence: Synthetic polypeptide”
DVVMTQSPLTLSVAIGQSASISCKSSQSLLDSDGKTYLNWLLQRPGQSPKRLISLVSKLDSGVPDRFTGSGSGTDFTLRISRVEAEDLGIYYCWQGTHFPGTFGGGTKLEIK

SEQ ID NO: 26 - protein, 16 aa, Artificial Sequence
  source: /note=“Description of Artificial Sequence: Synthetic peptide”
KSSQSLLDSDGKTYLN

SEQ ID NO: 27 - protein, 7 aa, Artificial Sequence
  source: /note=“Description of Artificial Sequence: Synthetic peptide”
LVSKLDS

SEQ ID NO: 28 - protein, 9 aa, Artificial Sequence
  source: /note=“Description of Artificial Sequence: Synthetic peptide”
WQGTHFPGT

SEQ ID NO: 29 - protein, 114 aa, Artificial Sequence; identical to SEQ ID NO: 21
  source: /note=“Description of Artificial Sequence: Synthetic polypeptide”

SEQ ID NO: 30 - protein, 7 aa, Artificial Sequence
  source: /note=“Description of Artificial Sequence: Synthetic peptide”
GFNIEDY

SEQ ID NO: 31 - protein, 6 aa, Artificial Sequence
  source: /note=“Description of Artificial Sequence: Synthetic peptide”
DPENDE

SEQ ID NO: 32 - protein, 5 aa, Artificial Sequence
  source: /note=“Description of Artificial Sequence: Synthetic peptide”
RGGVY

SEQ ID NO: 33 - protein, 112 aa, Artificial Sequence; identical to SEQ ID NO: 25
  source: /note=“Description of Artificial Sequence: Synthetic polypeptide”

SEQ ID NO: 34 - protein, 12 aa, Artificial Sequence
  source: /note=“Description of Artificial Sequence: Synthetic peptide”
SQSLLDSDGKTY

SEQ ID NO: 35 - protein, 3 aa, Artificial Sequence
  source: /note=“Description of Artificial Sequence: Synthetic peptide”
LVS

SEQ ID NO: 36 - protein, 6 aa, Artificial Sequence
  source: /note=“Description of Artificial Sequence: Synthetic peptide”
GTHFPG

SEQ ID NO: 37 - protein, 5 aa, Artificial Sequence
  source: /note=“Description of Artificial Sequence: Synthetic peptide”
GGGGS

SEQ ID NO: 38 - protein, 246 aa, Artificial Sequence
  source: /note=“Description of Artificial Sequence: Synthetic polypeptide”
EIQLVQSGAEVKKPGATVKISCKGSGFNIEDYYIHWVQQAPGKGLEWMGRIDPENDETKYGPIFQGRVTITADTSTNTVYMELSSLRSEDTAVYYCAFRGGVYWGQGTTVTVSSGGGGSGGGGSGGGGSGGGGSDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIK

SEQ ID NO: 39 - DNA, 738 bp, Artificial Sequence
  source: /note=“Description of Artificial Sequence: Synthetic polynucleotide”
gaaatccagctggtccaatcgggagctgaggtcaagaagccgggagccaccgtcaagatctcatgcaaggggtcgggattcaacatcgaggactactacattcactgggtgcagcaagctccgggaaaaggcctggaatggatgggcagaatcgacccagaaaacgacgaaactaagtacggaccgattttccaaggaagagtgactatcaccgccgatacttcaaccaataccgtctacatggaactgagctcgctccggtccgaagatactgcagtgtattactgtgcctttcgcggaggggtgtactggggccaaggaactactgtcactgtctcgtcaggaggcggagggtcgggaggaggcgggagcggaggcggtggctcgggtggcggaggaagcgacgtggtgatgacccagtccccggactccctcgccgtgagcctcggagagagggcgactatcaattgcaagtcgtcccagtcacttctggattccgatggtaaaacgtacctcaactggctgcagcaaaagccagggcagccacccaaacggttgatctcccttgtgtccaaactggatagcggagtgcctgaccgcttctcgggttccggtagcgggaccgacttcaccctgacgatcagctcactgcaggcggaggacgtggcagtgtactactgctggcagggaacccacttccctggcacctttggaggtggcaccaaggtggagatcaag

SEQ ID NO: 40 - DNA, 831 bp, Artificial Sequence
  source: /note=“Description of Artificial Sequence: Synthetic polynucleotide”
atggccctccctgtcaccgccctgctgcttccgctggctcttctgctccacgccgctcggcccgaaatccagctggtccaatcgggagctgaggtcaagaagccgggagccaccgtcaagatctcatgcaaggggtcgggattcaacatcgaggactactacattcactgggtgcagcaagctccgggaaaaggcctggaatggatgggcagaatcgacccagaaaacgacgaaactaagtacggaccgattttccaaggaagagtgactatcaccgccgatacttcaaccaataccgtctacatggaactgagctcgctccggtccgaagatactgcagtgtattactgtgcctttcgcggaggggtgtactggggccaaggaactactgtcactgtctcgtcaggaggcggagggtcgggaggaggcgggagcggaggcggtggctcgggtggcggaggaagcgacgtggtgatgacccagtccccggactccctcgccgtgagcctcggagagagggcgactatcaattgcaagtcgtcccagtcacttctggattccgatggtaaaacgtacctcaactggctgcagcaaaagccagggcagccacccaaacggttgatctcccttgtgtccaaactggatagcggagtgcctgaccgcttctcgggttccggtagcgggaccgacttcaccctgacgatcagctcactgcaggcggaggacgtggcagtgtactactgctggcagggaacccacttccctggcacctttggaggtggcaccaaggtggagatcaagggatcgcaccaccatcaccatcatcatcac

SEQ ID NO: 41 - protein, 277 aa, Artificial Sequence
  source: /note=“Description of Artificial Sequence: Synthetic polypeptide”
MALPVTALLLPLALLLHAARPEIQLVQSGAEVKKPGATVKISCKGSGFNIEDYYIHWVQQAPGKGLEWMGRIDPENDETKYGPIFQGRVTITADTSTNTVYMELSSLRSEDTAVYYCAFRGGVYWGQGTTVTVSSGGGGSGGGGSGGGGSGGGGSDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIKGSHHHHHHHH

SEQ ID NO: 42 - DNA, 1470 bp, Artificial Sequence
  source: /note=“Description of Artificial Sequence: Synthetic polynucleotide”
atggccctccctgtcaccgccctgctgcttccgctggctcttctgctccacgccgctcggcccgagatccagctggtgcagtcgggagctgaagtcaaaaagcctggcgcaaccgtcaagatctcgtgcaaaggatcagggttcaacatcgaggactactacatccattgggtgcaacaggcacccggaaaaggcctggagtggatggggaggattgacccagaaaatgacgaaaccaagtacggaccgatcttccaaggacgggtgaccatcacggctgacacttccactaacaccgtctacatggaactctcgagccttcgctcggaagataccgcggtgtactactgcgcctttagaggtggagtctactggggacaagggactaccgtcaccgtgtcgtcaggtggcggaggatcaggcggaggcggctccggtggaggaggaagcggaggaggtggctccgacgtggtgatgacgcagtcaccggactccttggcggtgagcctgggtgaacgcgccactatcaactgcaagagctcccagagcttgctggactccgatggaaagacttatctcaattggctgcaacagaagcctggccagccgccaaagagactcatctcactggtgagcaagctggatagcggagtgccagatcggttttcgggatcgggctcaggcaccgacttcaccctgactatttcctccctccaagccgaggatgtggccgtctactactgttggcaggggactcacttcccggggaccttcggtggaggcactaaggtggagatcaaaaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 43 - protein, 490 aa, Artificial Sequence
  source: /note=“Description of Artificial Sequence: Synthetic polypeptide”
MALPVTALLLPLALLLHAARPEIQLVQSGAEVKKPGATVKISCKGSGFNIEDYYIHWVQQAPGKGLEWMGRIDPENDETKYGPIFQGRVTITADTSTNTVYMELSSLRSEDTAVYYCAFRGGVYWGQGTTVTVSSGGGGSGGGGSGGGGSGGGGSDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 246 aa, Artificial Sequence
  source: /note=“Description of Artificial Sequence: Synthetic polypeptide”
DVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIKGGGGSGGGGSGGGGSGGGGSEIQLVQSGAEVKKPGATVKISCKGSGFNIEDYYIHWVQQAPGKGLEWMGRIDPENDETKYGPIFQGRVTITADTSTNTVYMELSSLRSEDTAVYYCAFRGGVYWGQGTTVTVSS

SEQ ID NO: 45 - DNA, 738 bp, Artificial Sequence
  source: /note=“Description of Artificial Sequence: Synthetic polynucleotide”
gatgtcgtgatgacccagtccccagactccctcgcagtgtccttgggagaacgggccaccatcaactgcaaatcgagccagtcactgctggactcagacggaaagacctacctcaactggctgcagcagaagcctggccagccaccgaagcgcctgatctccctggtgtccaagctggactcgggcgtcccggacaggtttagcggtagcggctcgggaaccgacttcactctgaccattagctcgctccaagctgaagatgtggcggtctactactgctggcaggggacccacttccccgggacctttggcggaggaactaaagtcgaaatcaaaggaggaggcggatcaggtggaggaggcagcggaggaggagggagcggcggtggcggctccgaaattcaacttgtgcaatccggtgccgaggtgaagaaacctggtgccactgtcaagatctcgtgtaagggatcgggattcaatatcgaggactactacatccactgggtgcaacaggcgccaggaaagggattggagtggatgggtcgcatcgacccggaaaacgatgagactaagtacggaccgatcttccaaggccgggtcacgatcactgcggatacctccactaataccgtgtatatggagctctcgtcactgagaagcgaagatacggccgtgtactactgcgcattcagaggaggtgtgtactggggccagggaactactgtgaccgtgtcgtcg

SEQ ID NO: 46 - DNA, 831 bp, Artificial Sequence
  source: /note=“Description of Artificial Sequence: Synthetic polynucleotide”
atggccctccctgtcaccgccctgctgcttccgctggctcttctgctccacgccgctcggcccgatgtcgtgatgacccagtccccagactccctcgcagtgtccttgggagaacgggccaccatcaactgcaaatcgagccagtcactgctggactcagacggaaagacctacctcaactggctgcagcagaagcctggccagccaccgaagcgcctgatctccctggtgtccaagctggactcgggcgtcccggacaggtttagcggtagcggctcgggaaccgacttcactctgaccattagctcgctccaagctgaagatgtggcggtctactactgctggcaggggacccacttccccgggacctttggcggaggaactaaagtcgaaatcaaaggaggaggcggatcaggtggaggaggcagcggaggaggagggagcggcggtggcggctccgaaattcaacttgtgcaatccggtgccgaggtgaagaaacctggtgccactgtcaagatctcgtgtaagggatcgggattcaatatcgaggactactacatccactgggtgcaacaggcgccaggaaagggattggagtggatgggtcgcatcgacccggaaaacgatgagactaagtacggaccgatcttccaaggccgggtcacgatcactgcggatacctccactaataccgtgtatatggagctctcgtcactgagaagcgaagatacggccgtgtactactgcgcattcagaggaggtgtgtactggggccagggaactactgtgaccgtgtcgtcggggtcacatcaccaccatcatcatcaccac

SEQ ID NO: 47 - protein, 277 aa, Artificial Sequence
  source: /note=“Description of Artificial Sequence: Synthetic polypeptide”
MALPVTALLLPLALLLHAARPDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIKGGGGSGGGGSGGGGSGGGGSEIQLVQSGAEVKKPGATVKISCKGSGFNIEDYYIHWVQQAPGKGLEWMGRIDPENDETKYGPIFQGRVTITADTSTNTVYMELSSLRSEDTAVYYCAFRGGVYWGQGTTVTVSSGSHHHHHHHH

SEQ ID NO: 48 - DNA, 1470 bp, Artificial Sequence
  source: /note=“Description of Artificial Sequence: Synthetic polynucleotide”
atggccctccctgtcaccgccctgctgcttccgctggctcttctgctccacgccgctcggcccgacgtggtcatgactcaaagcccagattccttggctgtctcccttggagaaagagcaacgatcaattgcaaaagctcgcagtccctgttggactccgatggaaaaacctacctcaactggctgcagcagaagccgggacaaccaccaaagcggctgatttccctcgtgtccaagctggacagcggcgtgccggatcgcttctcgggcagcggctcgggaaccgattttactctcactatttcgtcactgcaagcggaggacgtggcggtgtattactgctggcagggcactcacttcccgggtacttttggtggaggtaccaaagtcgaaatcaagggtggaggcgggagcggaggaggcgggtcgggaggaggaggatcgggtggcggaggctcagaaatccagctggtgcagtcaggtgccgaagtgaagaagcctggggccacggtgaagatctcgtgcaaggggagcggattcaacatcgaggattactacatccattgggtgcaacaggcccctggcaaagggctggaatggatgggaaggatcgaccccgagaatgacgagactaagtacggcccgatcttccaaggacgggtgaccatcactgcagacacttcaaccaacaccgtctacatggaactctcctcgctgcgctccgaggacaccgccgtgtactactgtgctttcagaggaggagtctactggggacagggaacgaccgtgaccgtcagctcaaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 49 - protein, 490 aa, Artificial Sequence
  source: /note=“Description of Artificial Sequence: Synthetic polypeptide”
MALPVTALLLPLALLLHAARPDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIKGGGGSGGGGSGGGGSGGGGSEIQLVQSGAEVKKPGATVKISCKGSGFNIEDYYIHWVQQAPGKGLEWMGRIDPENDETKYGPIFQGRVTITADTSTNTVYMELSSLRSEDTAVYYCAFRGGVYWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 246 aa, Artificial Sequence
  source: /note=“Description of Artificial Sequence: Synthetic polypeptide”
EIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGGGGSGGGGSGGGGSGGGGSDVVMTQSPLSLPVTLGQPASISCKSSQSLLDSDGKTYLNWLQQRPGQSPRRLISLVSKLDSGVPDRFSGSGSGTDFTLKISRVEAEDVGVYYCWQGTHFPGTFGGGTKVEIK

SEQ ID NO: 51 - DNA, 738 bp, Artificial Sequence
  source: /note=“Description of Artificial Sequence: Synthetic polynucleotide”
gagattcagctggtccaaagcggcgcagaagtgaaaaagccaggggaatcgttgcgcatcagctgtaaaggttccggcttcaacatcgaggactattacatccattgggtgcggcagatgccaggaaaggggctggaatggatgggacggattgacccggagaacgacgaaaccaagtacggaccgatctttcaaggacacgtgactatctccgccgacaccagcatcaatacggtgtacctccaatggtcctcactcaaggcctcggataccgcgatgtactactgcgcgttcagaggaggcgtctactggggacaagggactactgtgactgtctcatcaggaggtggaggaagcggaggaggtggctcgggcggaggtggatcgggaggaggagggtccgatgtggtgatgacccagtccccactgtcgctcccggtgaccctcggacagcctgctagcatctcgtgcaaatcctcgcaatccctgctggactcggacggaaaaacgtacctcaattggctgcagcagcgccctggccagagcccgagaaggcttatctcgctggtgtcaaagctggatagcggtgtgcccgaccggttcagcggctcagggtcaggaaccgatttcaccttgaagatctcccgcgtggaagccgaagatgtcggagtctactactgctggcagggtactcacttcccggggacctttggtggcggcactaaggtcgagattaag

SEQ ID NO: 52 - DNA, 831 bp, Artificial Sequence
  source: /note=“Description of Artificial Sequence: Synthetic polynucleotide”
atggccctccctgtcaccgccctgctgcttccgctggctcttctgctccacgccgctcggcccgagattcagctggtccaaagcggcgcagaagtgaaaaagccaggggaatcgttgcgcatcagctgtaaaggttccggcttcaacatcgaggactattacatccattgggtgcggcagatgccaggaaaggggctggaatggatgggacggattgacccggagaacgacgaaaccaagtacggaccgatctttcaaggacacgtgactatctccgccgacaccagcatcaatacggtgtacctccaatggtcctcactcaaggcctcggataccgcgatgtactactgcgcgttcagaggaggcgtctactggggacaagggactactgtgactgtctcatcaggaggtggaggaagcggaggaggtggctcgggcggaggtggatcgggaggaggagggtccgatgtggtgatgacccagtccccactgtcgctcccggtgaccctcggacagcctgctagcatctcgtgcaaatcctcgcaatccctgctggactcggacggaaaaacgtacctcaattggctgcagcagcgccctggccagagcccgagaaggcttatctcgctggtgtcaaagctggatagcggtgtgcccgaccggttcagcggctcagggtcaggaaccgatttcaccttgaagatctcccgcgtggaagccgaagatgtcggagtctactactgctggcagggtactcacttcccggggacctttggtggcggcactaaggtcgagattaagggctcacaccatcatcaccatcaccaccac

SEQ ID NO: 53 - protein, 277 aa, Artificial Sequence
  source: /note=“Description of Artificial Sequence: Synthetic polypeptide”
MALPVTALLLPLALLLHAARPEIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGGGGSGGGGSGGGGSGGGGSDVVMTQSPLSLPVTLGQPASISCKSSQSLLDSDGKTYLNWLQQRPGQSPRRLISLVSKLDSGVPDRFSGSGSGTDFTLKISRVEAEDVGVYYCWQGTHFPGTFGGGTKVEIKGSHHHHHHHH

SEQ ID NO: 54 - DNA, 1470 bp, Artificial Sequence
  source: /note=“Description of Artificial Sequence: Synthetic polynucleotide”
atggccctccctgtcaccgccctgctgcttccgctggctcttctgctccacgccgctcggcccgaaatccagctggtgcaaagcggagccgaggtgaagaagcccggagaatccctgcgcatctcgtgtaagggttccggctttaacatcgaggattactacatccactgggtgagacagatgccgggcaaaggtctggaatggatgggccgcatcgacccggagaacgacgaaaccaaatacggaccaatcttccaaggacatgtgactatttccgcggatacctccatcaacactgtctacttgcagtggagctcgctcaaggcgtcggataccgccatgtactactgcgcattcagaggaggtgtgtactggggccagggcactacggtcaccgtgtcctcgggaggtggagggtcaggaggcggaggctcgggcggtggaggatcaggcggaggaggaagcgatgtggtcatgactcaatccccactgtcactgcctgtcactctggggcaaccggcttccatctcatgcaagtcaagccaatcgctgctcgactccgacggaaaaacctacctcaattggcttcagcagcgcccaggccagtcgcctcggaggctgatctcactcgtgtcgaagcttgactccggggtgccggatcggtttagcggaagcggatcggggaccgacttcacgttgaagattagccgggtggaagccgaggacgtgggagtctattactgctggcaggggacccacttcccggggactttcggaggaggcaccaaagtcgagattaagaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 55 - protein, 490 aa, Artificial Sequence
  source: /note=“Description of Artificial Sequence: Synthetic polypeptide”
MALPVTALLLPLALLLHAARPEIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGGGGSGGGGSGGGGSGGGGSDVVMTQSPLSLPVTLGQPASISCKSSQSLLDSDGKTYLNWLQQRPGQSPRRLISLVSKLDSGVPDRFSGSGSGTDFTLKISRVEAEDVGVYYCWQGTHFPGTFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - protein, 246 aa, Artificial Sequence
  source: /note=“Description of Artificial Sequence: Synthetic polypeptide”
DVVMTQSPLSLPVTLGQPASISCKSSQSLLDSDGKTYLNWLQQRPGQSPRRLISLVSKLDSGVPDRFSGSGSGTDFTLKISRVEAEDVGVYYCWQGTHFPGTFGGGTKVEIKGGGGSGGGGSGGGGSGGGGSEIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSS

SEQ ID NO: 57 - DNA, 738 bp, Artificial Sequence
  source: /note=“Description of Artificial Sequence: Synthetic polynucleotide”
gacgtcgtcatgacccagagcccgctgtcactgcctgtgaccctgggccagccggcgtccattagctgcaaatcctcgcaatccctgctcgactcagacggaaaaacgtacttgaactggctccaacagcgccctgggcaatccccaaggcggcttatctcactcgtcagcaagctcgatagcggtgtcccagacagattttcgggctcgggatcgggcactgatttcactctgaagatctcgcgggtggaagccgaggatgtgggagtgtactattgctggcagggcactcacttccccgggacgtttggcggaggaactaaggtcgagatcaaaggaggaggtggatcaggcggaggtgggagcggaggaggaggaagcggtggtggaggttccgaaatccagctggtgcaatcaggagccgaggtgaagaagccgggagaatccctgcgcatctcgtgcaagggctcgggcttcaacatcgaggattactacatccactgggtgcggcagatgccgggaaaggggttggaatggatgggacgcattgacccggaaaatgatgaaaccaaatacgggccaatcttccaaggccacgtgaccattagcgctgacacttccatcaacaccgtgtaccttcagtggtcctcactgaaggcgtcggacactgccatgtactactgtgcattcagaggaggggtctactggggacagggcaccaccgtgaccgtgagctcc

SEQ ID NO: 58 - DNA, 831 bp, Artificial Sequence
  source: /note=“Description of Artificial Sequence: Synthetic polynucleotide”
atggccctccctgtcaccgccctgctgcttccgctggctcttctgctccacgccgctcggcccgacgtcgtcatgacccagagcccgctgtcactgcctgtgaccctgggccagccggcgtccattagctgcaaatcctcgcaatccctgctcgactcagacggaaaaacgtacttgaactggctccaacagcgccctgggcaatccccaaggcggcttatctcactcgtcagcaagctcgatagcggtgtcccagacagattttcgggctcgggatcgggcactgatttcactctgaagatctcgcgggtggaagccgaggatgtgggagtgtactattgctggcagggcactcacttccccgggacgtttggcggaggaactaaggtcgagatcaaaggaggaggtggatcaggcggaggtgggagcggaggaggaggaagcggtggtggaggttccgaaatccagctggtgcaatcaggagccgaggtgaagaagccgggagaatccctgcgcatctcgtgcaagggctcgggcttcaacatcgaggattactacatccactgggtgcggcagatgccgggaaaggggttggaatggatgggacgcattgacccggaaaatgatgaaaccaaatacgggccaatcttccaaggccacgtgaccattagcgctgacacttccatcaacaccgtgtaccttcagtggtcctcactgaaggcgtcggacactgccatgtactactgtgcattcagaggaggggtctactggggacagggcaccaccgtgaccgtgagctccggctcgcatcaccatcatcaccaccatcac

SEQ ID NO: 59 - protein, 277 aa, Artificial Sequence
  source: /note=“Description of Artificial Sequence: Synthetic polypeptide”
MALPVTALLLPLALLLHAARPDVVMTQSPLSLPVTLGQPASISCKSSQSLLDSDGKTYLNWLQQRPGQSPRRLISLVSKLDSGVPDRFSGSGSGTDFTLKISRVEAEDVGVYYCWQGTHFPGTFGGGTKVEIKGGGGSGGGGSGGGGSGGGGSEIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGSHHHHHHHH

SEQ ID NO: 60 - DNA, 1470 bp, Artificial Sequence
  source: /note=“Description of Artificial Sequence: Synthetic polynucleotide”
atggccctccctgtcaccgccctgctgcttccgctggctcttctgctccacgccgctcggcccgacgtcgtcatgacccaatcccctctctccctgccggtcaccctgggtcagccggcgtcgatctcatgcaaaagctcacagtccctgctggattcggacggaaaaacctacttgaactggctccaacagaggccgggtcagtcccctcgcagactgatctcgctggtgagcaagctcgactcgggtgtgccggatcggttctccgggtcaggatcgggcaccgactttacgctcaagatttcgagagtggaggccgaggatgtgggagtgtactattgctggcagggcacgcatttccccgggacctttggaggcgggactaaggtggaaatcaagggaggtggcggatcaggcggaggaggcagcggcggaggtggatcaggaggcggagggtcagagatccagctggtccaaagcggagcagaggtgaagaagccaggcgagtcccttcgcatttcgtgcaaagggagcggcttcaacattgaagattactacatccactgggtgcggcaaatgccaggaaagggtctggaatggatgggacggatcgacccagaaaatgatgaaactaagtacggaccgatcttccaaggacacgtcactatctccgcggacacttcgatcaacaccgtgtacctccagtggagcagcttgaaagcctccgacaccgctatgtactactgtgccttccgcggaggagtctactggggacaggggactactgtgaccgtgtcgtccaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 61 - protein, 490 aa, Artificial Sequence
  source: /note=“Description of Artificial Sequence: Synthetic polypeptide”
MALPVTALLLPLALLLHAARPDVVMTQSPLSLPVTLGQPASISCKSSQSLLDSDGKTYLNWLQQRPGQSPRRLISLVSKLDSGVPDRFSGSGSGTDFTLKISRVEAEDVGVYYCWQGTHFPGTFGGGTKVEIKGGGGSGGGGSGGGGSGGGGSEIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 246 aa, Artificial Sequence
  source: /note=“Description of Artificial Sequence: Synthetic polypeptide”
EIQLVQSGAEVKKPGATVKISCKGSGFNIEDYYIHWVQQAPGKGLEWMGRIDPENDETKYGPIFQGRVTITADTSTNTVYMELSSLRSEDTAVYYCAFRGGVYWGQGTTVTVSSGGGGSGGGGSGGGGSGGGGSDVVMTQSPLSLPVTLGQPASISCKSSQSLLDSDGKTYLNWLQQRPGQSPRRLISLVSKLDSGVPDRFSGSGSGTDFTLKISRVEAEDVGVYYCWQGTHFPGTFGGGTKVEIK

SEQ ID NO: 63 - DNA, 738 bp, Artificial Sequence
  source: /note=“Description of Artificial Sequence: Synthetic polynucleotide”
gaaatccagctcgtgcagagcggagccgaggtcaagaaaccgggtgctaccgtgaagatttcatgcaagggatcgggcttcaacatcgaggattactacatccactgggtgcagcaggcaccaggaaaaggacttgaatggatgggccggatcgacccggaaaatgacgagactaagtacggccctatcttccaaggacgggtgacgatcaccgcagacactagcaccaacaccgtctatatggaactctcgtccctgaggtccgaagatactgccgtgtactactgtgcgtttcgcggaggtgtgtactggggacagggtaccaccgtcaccgtgtcatcgggcggtggaggctccggtggaggagggtcaggaggcggtggaagcggaggaggcggcagcgacgtggtcatgactcaatcgccgctgtcgctgcccgtcactctgggacaacccgcgtccatcagctgcaaatcctcgcagtcactgcttgactccgatggaaagacctacctcaactggctgcagcaacgcccaggccaatccccaagacgcctgatctcgttggtgtcaaagctggactcaggggtgccggaccggttctccgggagcgggtcgggcacggatttcactctcaagatctccagagtggaagccgaggatgtgggagtctactactgctggcagggaacccatttccctggaacttttggcggaggaactaaggtcgagattaaa

SEQ ID NO: 64 - DNA, 831 bp, Artificial Sequence
  source: /note=“Description of Artificial Sequence: Synthetic polynucleotide”
atggccctccctgtcaccgccctgctgcttccgctggctcttctgctccacgccgctcggcccgaaatccagctcgtgcagagcggagccgaggtcaagaaaccgggtgctaccgtgaagatttcatgcaagggatcgggcttcaacatcgaggattactacatccactgggtgcagcaggcaccaggaaaaggacttgaatggatgggccggatcgacccggaaaatgacgagactaagtacggccctatcttccaaggacgggtgacgatcaccgcagacactagcaccaacaccgtctatatggaactctcgtccctgaggtccgaagatactgccgtgtactactgtgcgtttcgcggaggtgtgtactggggacagggtaccaccgtcaccgtgtcatcgggcggtggaggctccggtggaggagggtcaggaggcggtggaagcggaggaggcggcagcgacgtggtcatgactcaatcgccgctgtcgctgcccgtcactctgggacaacccgcgtccatcagctgcaaatcctcgcagtcactgcttgactccgatggaaagacctacctcaactggctgcagcaacgcccaggccaatccccaagacgcctgatctcgttggtgtcaaagctggactcaggggtgccggaccggttctccgggagcgggtcgggcacggatttcactctcaagatctccagagtggaagccgaggatgtgggagtctactactgctggcagggaacccatttccctggaacttttggcggaggaactaaggtcgagattaaagggagccaccatcatcatcaccaccaccac

SEQ ID NO: 65 - protein, 277 aa, Artificial Sequence
  source: /note=“Description of Artificial Sequence: Synthetic polypeptide”
MALPVTALLLPLALLLHAARPEIQLVQSGAEVKKPGATVKISCKGSGFNIEDYYIHWVQQAPGKGLEWMGRIDPENDETKYGPIFQGRVTITADTSTNTVYMELSSLRSEDTAVYYCAFRGGVYWGQGTTVTVSSGGGGSGGGGSGGGGSGGGGSDVVMTQSPLSLPVTLGQPASISCKSSQSLLDSDGKTYLNWLQQRPGQSPRRLISLVSKLDSGVPDRFSGSGSGTDFTLKISRVEAEDVGVYYCWQGTHFPGTFGGGTKVEIKGSHHHHHHHH

SEQ ID NO: 66 - DNA, 1470 bp, Artificial Sequence
  source: /note=“Description of Artificial Sequence: Synthetic polynucleotide”
atggccctccctgtcaccgccctgctgcttccgctggctcttctgctccacgccgctcggcccgaaatccagctcgtgcagagcggagccgaggtcaagaaaccgggtgctaccgtgaagatttcatgcaagggatcgggcttcaacatcgaggattactacatccactgggtgcagcaggcaccaggaaaaggacttgaatggatgggccggatcgacccggaaaatgacgagactaagtacggccctatcttccaaggacgggtgacgatcaccgcagacactagcaccaacaccgtctatatggaactctcgtccctgaggtccgaagatactgccgtgtactactgtgcgtttcgcggaggtgtgtactggggacagggtaccaccgtcaccgtgtcatcgggcggtggaggctccggtggaggagggtcaggaggcggtggaagcggaggaggcggcagcgacgtggtcatgactcaatcgccgctgtcgctgcccgtcactctgggacaacccgcgtccatcagctgcaaatcctcgcagtcactgcttgactccgatggaaagacctacctcaactggctgcagcaacgcccaggccaatccccaagacgcctgatctcgttggtgtcaaagctggactcaggggtgccggaccggttctccgggagcgggtcgggcacggatttcactctcaagatctccagagtggaagccgaggatgtgggagtctactactgctggcagggaacccatttccctggaacttttggcggaggaactaaggtcgagattaaaaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 67 - protein, 490 aa, Artificial Sequence
  source: /note=“Description of Artificial Sequence: Synthetic polypeptide”
MALPVTALLLPLALLLHAARPEIQLVQSGAEVKKPGATVKISCKGSGFNIEDYYIHWVQQAPGKGLEWMGRIDPENDETKYGPIFQGRVTITADTSTNTVYMELSSLRSEDTAVYYCAFRGGVYWGQGTTVTVSSGGGGSGGGGSGGGGSGGGGSDVVMTQSPLSLPVTLGQPASISCKSSQSLLDSDGKTYLNWLQQRPGQSPRRLISLVSKLDSGVPDRFSGSGSGTDFTLKISRVEAEDVGVYYCWQGTHFPGTFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - protein, 246 aa, Artificial Sequence
  source: /note=“Description of Artificial Sequence: Synthetic polypeptide”
EIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGGGGSGGGGSGGGGSGGGGSDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIK

SEQ ID NO: 69 - DNA, 738 bp, Artificial Sequence
  source: /note=“Description of Artificial Sequence: Synthetic polynucleotide”
gaaatccagctggtgcagtcaggcgccgaggtcaagaagccgggagagtcgctgagaatctcgtgcaagggctcggggttcaacatcgaggactactacattcactgggtcaggcagatgccgggaaagggactggaatggatgggccggatcgacccagaaaatgacgaaaccaaatacgggccgatttttcaaggccacgtgactatcagcgcagacacgagcatcaacactgtctacctccagtggtcctcgcttaaggccagcgataccgctatgtactactgcgcattcagaggcggggtgtactggggacaaggaaccactgtgaccgtgagcagcggaggtggcggctcgggaggaggtgggagcggaggaggaggttccggcggtggaggatcagatgtcgtgatgacccagtccccggactccctcgctgtctcactgggcgagcgcgcgaccatcaactgcaaatcgagccagtcgctgttggactccgatggaaagacttatctgaattggctgcaacagaaaccaggacaacctcccaagcggctcatctcgcttgtgtcaaaactcgattcgggagtgccagaccgcttctcggggtccgggagcggaactgactttactttgaccatttcctcactgcaagcggaggatgtggccgtgtattactgttggcagggcacgcatttccctggaaccttcggtggcggaactaaggtggaaatcaag

SEQ ID NO: 70 - DNA, 834 bp, Artificial Sequence
  source: /note=“Description of Artificial Sequence: Synthetic polynucleotide”
atggccctccctgtcaccgccctgctgcttccgctggctcttctgctccacgccgctcggcccgaaatccagctggtgcagtcaggcgccgaggtcaagaagccgggagagtcgctgagaatctcgtgcaagggctcggggttcaacatcgaggactactacattcactgggtcaggcagatgccgggaaagggactggaatggatgggccggatcgacccagaaaatgacgaaaccaaatacgggccgatttttcaaggccacgtgactatcagcgcagacacgagcatcaacactgtctacctccagtggtcctcgcttaaggccagcgataccgctatgtactactgcgcattcagaggcggggtgtactggggacaaggaaccactgtgaccgtgagcagcggaggtggcggctcgggaggaggtgggagcggaggaggaggttccggcggtggaggatcagatgtcgtgatgacccagtccccggactccctcgctgtctcactgggcgagcgcgcgaccatcaactgcaaatcgagccagtcgctgttggactccgatggaaagacttatctgaattggctgcaacagaaaccaggacaacctcccaagcggctcatctcgcttgtgtcaaaactcgattcgggagtgccagaccgcttctcggggtccgggagcggaactgactttactttgaccatttcctcactgcaagcggaggatgtggccgtgtattactgttggcagggcacgcatttccctggaaccttcggtggcggaactaaggtggaaatcaagggatcacaccaccatcatcaccatcaccaccat

SEQ ID NO: 71 - protein, 278 aa, Artificial Sequence
  source: /note=“Description of Artificial Sequence: Synthetic polypeptide”
MALPVTALLLPLALLLHAARPEIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGGGGSGGGGSGGGGSGGGGSDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIKGSHHHHHHHHH

SEQ ID NO: 72 - DNA, 1470 bp, Artificial Sequence
  source: /note=“Description of Artificial Sequence: Synthetic polynucleotide”
atggccctccctgtcaccgccctgctgcttccgctggctcttctgctccacgccgctcggcccgagattcagctcgtgcaatcgggagcggaagtcaagaagccaggagagtccttgcggatctcatgcaagggtagcggctttaacatcgaggattactacatccactgggtgaggcagatgccggggaagggactcgaatggatgggacggatcgacccagaaaacgacgaaactaagtacggtccgatcttccaaggccatgtgactattagcgccgatacttcaatcaataccgtgtatctgcaatggtcctcattgaaagcctcagataccgcgatgtactactgtgctttcagaggaggggtctactggggacagggaactaccgtgactgtctcgtccggcggaggcgggtcaggaggtggcggcagcggaggaggagggtccggcggaggtgggtccgacgtcgtgatgacccagagccctgacagcctggcagtgagcctgggcgaaagagctaccattaactgcaaatcgtcgcagagcctgctggactcggacggaaaaacgtacctcaattggctgcagcaaaagcctggccagccaccgaagcgccttatctcactggtgtcgaagctggattcgggagtgcccgatcgcttctccggctcgggatcgggtactgacttcaccctcactatctcctcgcttcaagcagaggacgtggccgtctactactgctggcagggaacccactttccgggaaccttcggcggagggacgaaagtggagatcaagaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 73 - protein, 490 aa, Artificial Sequence
  source: /note=“Description of Artificial Sequence: Synthetic polypeptide”
MALPVTALLLPLALLLHAARPEIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGGGGSGGGGSGGGGSGGGGSDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - protein, 246 aa, Artificial Sequence
  source: /note=“Description of Artificial Sequence: Synthetic polypeptide”
DVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIKGGGGSGGGGSGGGGSGGGGSEIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSS

SEQ ID NO: 75 - DNA, 738 bp, Artificial Sequence
  source: /note=“Description of Artificial Sequence: Synthetic polynucleotide”
gacgtggtgatgacccaatcgccagattccctggcagtgtccctgggcgaacgcgccactattaactgcaaatcgtcacagtccttgcttgattccgacggaaagacctacctcaattggctccagcagaagccaggacaaccgccaaagagactgatctccctggtgtcaaagctggactcgggagtgcctgatcggttctcgggtagcgggagcggcaccgacttcactctgaccatctcgtcactccaggctgaggacgtggccgtgtattactgttggcagggtactcactttccgggcactttcggaggcggcaccaaggtggagattaaaggaggaggcggaagcggaggtggaggatcgggaggtggtgggagcggcggaggagggagcgagatccagctcgtccaatcgggagcggaagtgaagaagcccggagagtcacttagaatctcatgcaaggggtcgggcttcaacatcgaggattactacatccattgggtccgccagatgcctggtaaaggactggaatggatggggaggattgacccggaaaacgacgaaactaagtacggaccgatctttcaagggcacgtgactatctccgctgatacctcaatcaatactgtctacctccagtggtcctcgctgaaagcaagcgacaccgcgatgtactactgcgccttccggggaggagtgtactggggccaaggcaccacggtcacggtcagctcc

SEQ ID NO: 76 - DNA, 834 bp, Artificial Sequence
  source: /note=“Description of Artificial Sequence: Synthetic polynucleotide”
atggccctccctgtcaccgccctgctgcttccgctggctcttctgctccacgccgctcggcccgacgtggtgatgacccaatcgccagattccctggcagtgtccctgggcgaacgcgccactattaactgcaaatcgtcacagtccttgcttgattccgacggaaagacctacctcaattggctccagcagaagccaggacaaccgccaaagagactgatctccctggtgtcaaagctggactcgggagtgcctgatcggttctcgggtagcgggagcggcaccgacttcactctgaccatctcgtcactccaggctgaggacgtggccgtgtattactgttggcagggtactcactttccgggcactttcggaggcggcaccaaggtggagattaaaggaggaggcggaagcggaggtggaggatcgggaggtggtgggagcggcggaggagggagcgagatccagctcgtccaatcgggagcggaagtgaagaagcccggagagtcacttagaatctcatgcaaggggtcgggcttcaacatcgaggattactacatccattgggtccgccagatgcctggtaaaggactggaatggatggggaggattgacccggaaaacgacgaaactaagtacggaccgatctttcaagggcacgtgactatctccgctgatacctcaatcaatactgtctacctccagtggtcctcgctgaaagcaagcgacaccgcgatgtactactgcgccttccggggaggagtgtactggggccaaggcaccacggtcacggtcagctccggctcccatcaccaccaccatcaccatcatcac

SEQ ID NO: 77 - protein, 278 aa, Artificial Sequence
  source: /note=“Description of Artificial Sequence: Synthetic polypeptide”
MALPVTALLLPLALLLHAARPDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIKGGGGSGGGGSGGGGSGGGGSEIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGSHHHHHHHHH

SEQ ID NO: 78 - DNA, 1470 bp, Artificial Sequence
  source: /note=“Description of Artificial Sequence: Synthetic polynucleotide”
atggccctccctgtcaccgccctgctgcttccgctggctcttctgctccacgccgctcggcccgacgtggtgatgactcagtcgcctgactcgctggctgtgtcccttggagagcgggccactatcaattgcaagtcatcccagtcgctgctggattccgacgggaaaacctacctcaattggctgcagcaaaaaccgggacagcctccaaagcggctcatcagcctggtgtccaagttggacagcggcgtgccagaccgcttctccggttcgggaagcggtactgatttcacgctgaccatctcatccctccaagcggaggatgtggcagtctactactgttggcagggcacgcattttccgggcacttttggaggagggaccaaggtcgaaatcaagggaggaggtggctcgggcggaggaggctcgggaggaggaggatcaggaggcggtggaagcgagattcaactggtccagagcggcgcagaagtcaagaagccgggtgaatcgctcagaatctcgtgcaaaggatcgggattcaacatcgaggactactacattcactgggtcagacaaatgccgggcaaagggctggaatggatggggaggatcgaccccgaaaacgatgaaaccaagtacggaccaatcttccaagggcacgtgaccatttcggcggacacctcaatcaacactgtgtacctccagtggagctcacttaaggccagcgataccgccatgtactattgcgctttccgcggaggggtgtactggggacagggcactactgtgaccgtgtcatccaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 79 - protein, 490 aa, Artificial Sequence
  source: /note=“Description of Artificial Sequence: Synthetic polypeptide”
MALPVTALLLPLALLLHAARPDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIKGGGGSGGGGSGGGGSGGGGSEIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 80 - protein, 246 aa, Artificial Sequence
  source: /note=“Description of Artificial Sequence: Synthetic polypeptide”
DVVMTQSPLSLPVTLGQPASISCKSSQSLLDSDGKTYLNWLQQRPGQSPRRLISLVSKLDSGVPDRFSGSGSGTDFTLKISRVEAEDVGVYYCWQGTHFPGTFGGGTKVEIKGGGGSGGGGSGGGGSGGGGSEIQLVQSGAEVKKPGATVKISCKGSGFNIEDYYIHWVQQAPGKGLEWMGRIDPENDETKYGPIFQGRVTITADTSTNTVYMELSSLRSEDTAVYYCAFRGGVYWGQGTTVTVSS

SEQ ID NO: 81 - DNA, 738 bp, Artificial Sequence
  source: /note=“Description of Artificial Sequence: Synthetic polynucleotide”
gatgtggtcatgacgcagtcaccactgtccctccccgtgacccttggacagccagcgtcgattagctgcaagtcatcccaatccctgctcgattcggatggaaagacctatctcaactggctgcagcaaagacccggtcagagccctaggagactcatctcgttggtgtcaaagctggacagcggagtgccggaccggttttccggttcgggatcggggacggacttcactctgaagatttcacgggtggaagctgaggatgtgggagtgtactactgctggcagggaacccatttccctggcacttttggcggaggaactaaggtcgaaatcaagggaggaggtggctcgggaggaggcggatcgggcggaggcgggagcggcggaggagggtccgaaatccaacttgtccagtcaggagccgaagtgaagaaaccgggagccaccgtcaaaatcagctgtaagggatcgggattcaatatcgaggactactacatccactgggtgcagcaagctccgggcaaaggactggagtggatggggcgcatcgacccagagaacgacgaaaccaaatacggcccgatcttccaagggcgggtgaccatcaccgcggacacctcaactaacactgtgtacatggagctgagctccctgcgctccgaagatactgcagtctactactgcgccttccgcggtggtgtgtactggggacagggcaccactgtgactgtcagctcg

SEQ ID NO: 82 - DNA, 831 bp, Artificial Sequence
  source: /note=“Description of Artificial Sequence: Synthetic polynucleotide”
atggccctccctgtcaccgccctgctgcttccgctggctcttctgctccacgccgctcggcccgatgtggtcatgacgcagtcaccactgtccctccccgtgacccttggacagccagcgtcgattagctgcaagtcatcccaatccctgctcgattcggatggaaagacctatctcaactggctgcagcaaagacccggtcagagccctaggagactcatctcgttggtgtcaaagctggacagcggagtgccggaccggttttccggttcgggatcggggacggacttcactctgaagatttcacgggtggaagctgaggatgtgggagtgtactactgctggcagggaacccatttccctggcacttttggcggaggaactaaggtcgaaatcaagggaggaggtggctcgggaggaggcggatcgggcggaggcgggagcggcggaggagggtccgaaatccaacttgtccagtcaggagccgaagtgaagaaaccgggagccaccgtcaaaatcagctgtaagggatcgggattcaatatcgaggactactacatccactgggtgcagcaagctccgggcaaaggactggagtggatggggcgcatcgacccagagaacgacgaaaccaaatacggcccgatcttccaagggcgggtgaccatcaccgcggacacctcaactaacactgtgtacatggagctgagctccctgcgctccgaagatactgcagtctactactgcgccttccgcggtggtgtgtactggggacagggcaccactgtgactgtcagctcggggtcccaccatcatcaccaccaccatcac

SEQ ID NO: 83 - protein, 277 aa, Artificial Sequence
  source: /note=“Description of Artificial Sequence: Synthetic polypeptide”
MALPVTALLLPLALLLHAARPDVVMTQSPLSLPVTLGQPASISCKSSQSLLDSDGKTYLNWLQQRPGQSPRRLISLVSKLDSGVPDRFSGSGSGTDFTLKISRVEAEDVGVYYCWQGTHFPGTFGGGTKVEIKGGGGSGGGGSGGGGSGGGGSEIQLVQSGAEVKKPGATVKISCKGSGFNIEDYYIHWVQQAPGKGLEWMGRIDPENDETKYGPIFQGRVTITADTSTNTVYMELSSLRSEDTAVYYCAFRGGVYWGQGTTVTVSSGSHHHHHHHH

SEQ ID NO: 84 - DNA, 1470 bp, Artificial Sequence
  source: /note=“Description of Artificial Sequence: Synthetic polynucleotide”
atggccctccctgtcaccgccctgctgcttccgctggctcttctgctccacgccgctcggcccgatgtggtcatgacgcagtcaccactgtccctccccgtgacccttggacagccagcgtcgattagctgcaagtcatcccaatccctgctcgattcggatggaaagacctatctcaactggctgcagcaaagacccggtcagagccctaggagactcatctcgttggtgtcaaagctggacagcggagtgccggaccggttttccggttcgggatcggggacggacttcactctgaagatttcacgggtggaagctgaggatgtgggagtgtactactgctggcagggaacccatttccctggcacttttggcggaggaactaaggtcgaaatcaagggaggaggtggctcgggaggaggcggatcgggcggaggcgggagcggcggaggagggtccgaaatccaacttgtccagtcaggagccgaagtgaagaaaccgggagccaccgtcaaaatcagctgtaagggatcgggattcaatatcgaggactactacatccactgggtgcagcaagctccgggcaaaggactggagtggatggggcgcatcgacccagagaacgacgaaaccaaatacggcccgatcttccaagggcgggtgaccatcaccgcggacacctcaactaacactgtgtacatggagctgagctccctgcgctccgaagatactgcagtctactactgcgccttccgcggtggtgtgtactggggacagggcaccactgtgactgtcagctcgaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 85 - protein, 490 aa, Artificial Sequence
  source: /note=“Description of Artificial Sequence: Synthetic polypeptide”
MALPVTALLLPLALLLHAARPDVVMTQSPLSLPVTLGQPASISCKSSQSLLDSDGKTYLNWLQQRPGQSPRRLISLVSKLDSGVPDRFSGSGSGTDFTLKISRVEAEDVGVYYCWQGTHFPGTFGGGTKVEIKGGGGSGGGGSGGGGSGGGGSEIQLVQSGAEVKKPGATVKISCKGSGFNIEDYYIHWVQQAPGKGLEWMGRIDPENDETKYGPIFQGRVTITADTSTNTVYMELSSLRSEDTAVYYCAFRGGVYWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 86 - protein, 243 aa, Artificial Sequence; identical to SEQ ID NO: 11
  source: /note=“Description of Artificial Sequence: Synthetic polypeptide”

SEQ ID NO: 87 - DNA, 822 bp, Artificial Sequence
  source: /note=“Description of Artificial Sequence: Synthetic polynucleotide”
atggccctccctgtcaccgccctgctgcttccgctggctcttctgctccacgccgctcggcccgagatccagctccaacagagcggagccgaactggtcaaaccgggagcgtcggtgaagttgtcatgcactggatcgggcttcaacatcgaggattactacatccactgggtcaagcaacgcaccgagcaggggctggaatggatcggacggatcgaccccgaaaacgatgaaaccaagtacgggcctatcttccaaggacgggccaccattacggctgacacgtcaagcaataccgtctacctccagctttccagcctgacctccgaggacactgccgtgtactactgcgccttcagaggaggcgtgtactggggaccaggaaccactttgaccgtgtccagcggaggcggtggatcaggaggaggaggctcaggcggtggcggctcgcacatggacgtggtcatgactcagtccccgctgaccctgtcggtggcaattggacagagcgcatccatctcgtgcaagagctcacagtcgctgctggattccgacggaaagacttatctgaactggctgctccaaagaccagggcaatcaccgaaacgccttatctccctggtgtcgaaactcgactcgggtgtgccggatcggtttaccggtagcgggtccggcacggacttcactctccgcatttcgagggtggaagcggaggatctcgggatctactactgttggcagggaacccacttccctgggacttttggaggcggaactaagctggaaatcaagggtagccatcaccatcaccaccaccatcat

SEQ ID NO: 88 - protein, 274 aa, Artificial Sequence
  source: /note=“Description of Artificial Sequence: Synthetic polypeptide”
MALPVTALLLPLALLLHAARPEIQLQQSGAELVKPGASVKLSCTGSGFNIEDYYIHWVKQRTEQGLEWIGRIDPENDETKYGPIFQGRATITADTSSNTVYLQLSSLTSEDTAVYYCAFRGGVYWGPGTTLTVSSGGGGSGGGGSGGGGSHMDVVMTQSPLTLSVAIGQSASISCKSSQSLLDSDGKTYLNWLLQRPGQSPKRLISLVSKLDSGVPDRFTGSGSGTDFTLRISRVEAEDLGIYYCWQGTHFPGTFGGGTKLEIKGSHHHHHHHH

SEQ ID NO: 89 - DNA, 1461 bp, Artificial Sequence
  source: /note=“Description of Artificial Sequence: Synthetic polynucleotide”
atggccctccctgtcaccgccctgctgcttccgctggctcttctgctccacgccgctcggcccgagatccagctccaacagagcggagccgaactggtcaaaccgggagcgtcggtgaagttgtcatgcactggatcgggcttcaacatcgaggattactacatccactgggtcaagcaacgcaccgagcaggggctggaatggatcggacggatcgaccccgaaaacgatgaaaccaagtacgggcctatcttccaaggacgggccaccattacggctgacacgtcaagcaataccgtctacctccagctttccagcctgacctccgaggacactgccgtgtactactgcgccttcagaggaggcgtgtactggggaccaggaaccactttgaccgtgtccagcggaggcggtggatcaggaggaggaggctcaggcggtggcggctcgcacatggacgtggtcatgactcagtccccgctgaccctgtcggtggcaattggacagagcgcatccatctcgtgcaagagctcacagtcgctgctggattccgacggaaagacttatctgaactggctgctccaaagaccagggcaatcaccgaaacgccttatctccctggtgtcgaaactcgactcgggtgtgccggatcggtttaccggtagcgggtccggcacggacttcactctccgcatttcgagggtggaagcggaggatctcgggatctactactgttggcagggaacccacttccctgggacttttggaggcggaactaagctggaaatcaagaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 90 - protein, 487 aa, Artificial Sequence
  source: /note=“Description of Artificial Sequence: Synthetic polypeptide”
MALPVTALLLPLALLLHAARPEIQLQQSGAELVKPGASVKLSCTGSGFNIEDYYIHWVKQRTEQGLEWIGRIDPENDETKYGPIFQGRATITADTSSNTVYLQLSSLTSEDTAVYYCAFRGGVYWGPGTTLTVSSGGGGSGGGGSGGGGSHMDVVMTQSPLTLSVAIGQSASISCKSSQSLLDSDGKTYLNWLLQRPGQSPKRLISLVSKLDSGVPDRFTGSGSGTDFTLRISRVEAEDLGIYYCWQGTHFPGTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 91 - protein, 240 aa, Artificial Sequence; identical to SEQ ID NO: 12
  source: /note=“Description of Artificial Sequence: Synthetic polypeptide”

SEQ ID NO: 92 - DNA, 720 bp, Artificial Sequence
  source: /note=“Description of Artificial Sequence: Synthetic polynucleotide”
gatatccaaatgactcagagcccttcatccctgagcgccagcgtcggagacagggtgaccatcacgtgccgggcatcccaaggcattagaaataacttggcgtggtatcagcaaaaaccaggaaaggccccgaagcgcctgatctacgcggcctccaaccttcagtcaggagtgccctcgcgcttcaccgggagcggtagcggaactgagtttacccttatcgtgtcgtccctgcagccagaggacttcgcgacctactactgcctccagcatcactcgtacccgttgacttcgggaggcggaaccaaggtcgaaatcaaacgcactggctcgacgtcagggtccggtaaaccgggatcgggagaaggatcggaagtccaagtgctggagagcggaggcggactcgtgcaacctggcgggtcgctgcggctcagctgtgccgcgtcgggttttactttcagctcgtacgctatgtcatgggtgcggcaggctccgggaaaggggctggaatgggtgtccgctatttccggctcgggtggaagcaccaattacgccgactccgtgaagggacgcttcaccatctcacgggataactccaagaatactctgtacctccagatgaactcgctgagagccgaggacaccgcagtgtactactgcgcagggtcaagcggctggtccgaatactggggacagggcaccctcgtcactgtcagctcc

SEQ ID NO: 93 - DNA, 807 bp, Artificial Sequence
  source: /note=“Description of Artificial Sequence: Synthetic polynucleotide”
atggccctccctgtcaccgccctgctgcttccgctggctcttctgctccacgccgctcggcccgatatccaaatgactcagagcccttcatccctgagcgccagcgtcggagacagggtgaccatcacgtgccgggcatcccaaggcattagaaataacttggcgtggtatcagcaaaaaccaggaaaggccccgaagcgcctgatctacgcggcctccaaccttcagtcaggagtgccctcgcgcttcaccgggagcggtagcggaactgagtttacccttatcgtgtcgtccctgcagccagaggacttcgcgacctactactgcctccagcatcactcgtacccgttgacttcgggaggcggaaccaaggtcgaaatcaaacgcactggctcgacgtcagggtccggtaaaccgggatcgggagaaggatcggaagtccaagtgctggagagcggaggcggactcgtgcaacctggcgggtcgctgcggctcagctgtgccgcgtcgggttttactttcagctcgtacgctatgtcatgggtgcggcaggctccgggaaaggggctggaatgggtgtccgctatttccggctcgggtggaagcaccaattacgccgactccgtgaagggacgcttcaccatctcacgggataactccaagaatactctgtacctccagatgaactcgctgagagccgaggacaccgcagtgtactactgcgcagggtcaagcggctggtccgaatactggggacagggcaccctcgtcactgtcagctcccatcaccatcaccaccaccatcac

SEQ ID NO: 94 - protein, 269 aa, Artificial Sequence
  source: /note=“Description of Artificial Sequence: Synthetic polypeptide”
MALPVTALLLPLALLLHAARPDIQMTQSPSSLSASVGDRVTITCRASQGIRNNLAWYQQKPGKAPKRLIYAASNLQSGVPSRFTGSGSGTEFTLIVSSLQPEDFATYYCLQHHSYPLTSGGGTKVEIKRTGSTSGSGKPGSGEGSEVQVLESGGGLVQPGGSLRLSCAASGFTFSSYAMSWVRQAPGKGLEWVSAISGSGGSTNYADSVKGRFTISRDNSKNTLYLQMNSLRAEDTAVYYCAGSSGWSEYWGQGTLVTVSSHHHHHHHH

SEQ ID NO: 95 - DNA, 1452 bp, Artificial Sequence
  source: /note=“Description of Artificial Sequence: Synthetic polynucleotide”
atggccctccctgtcaccgccctgctgcttccgctggctcttctgctccacgccgctcggcccgatatccaaatgactcagagcccttcatccctgagcgccagcgtcggagacagggtgaccatcacgtgccgggcatcccaaggcattagaaataacttggcgtggtatcagcaaaaaccaggaaaggccccgaagcgcctgatctacgcggcctccaaccttcagtcaggagtgccctcgcgcttcaccgggagcggtagcggaactgagtttacccttatcgtgtcgtccctgcagccagaggacttcgcgacctactactgcctccagcatcactcgtacccgttgacttcgggaggcggaaccaaggtcgaaatcaaacgcactggctcgacgtcagggtccggtaaaccgggatcgggagaaggatcggaagtccaagtgctggagagcggaggcggactcgtgcaacctggcgggtcgctgcggctcagctgtgccgcgtcgggttttactttcagctcgtacgctatgtcatgggtgcggcaggctccgggaaaggggctggaatgggtgtccgctatttccggctcgggtggaagcaccaattacgccgactccgtgaagggacgcttcaccatctcacgggataactccaagaatactctgtacctccagatgaactcgctgagagccgaggacaccgcagtgtactactgcgcagggtcaagcggctggtccgaatactggggacagggcaccctcgtcactgtcagctccaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 96 - protein, 484 aa, Artificial Sequence
  source: /note=“Description of Artificial Sequence: Synthetic polypeptide”
MALPVTALLLPLALLLHAARPDIQMTQSPSSLSASVGDRVTITCRASQGIRNNLAWYQQKPGKAPKRLIYAASNLQSGVPSRFTGSGSGTEFTLIVSSLQPEDFATYYCLQHHSYPLTSGGGTKVEIKRTGSTSGSGKPGSGEGSEVQVLESGGGLVQPGGSLRLSCAASGFTFSSYAMSWVRQAPGKGLEWVSAISGSGGSTNYADSVKGRFTISRDNSKNTLYLQMNSLRAEDTAVYYCAGSSGWSEYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 97 - DNA, 1183 bp, Artificial Sequence
  source: /note=“Description of Artificial Sequence: Synthetic polynucleotide”
gtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 98 - DNA, 729 bp, Artificial Sequence
  source: /note=“Description of Artificial Sequence: Synthetic polynucleotide”
gagatccagctccaacagagcggagccgaactggtcaaaccgggagcgtcggtgaagttgtcatgcactggatcgggcttcaacatcgaggattactacatccactgggtcaagcaacgcaccgagcaggggctggaatggatcggacggatcgaccccgaaaacgatgaaaccaagtacgggcctatcttccaaggacgggccaccattacggctgacacgtcaagcaataccgtctacctccagctttccagcctgacctccgaggacactgccgtgtactactgcgccttcagaggaggcgtgtactggggaccaggaaccactttgaccgtgtccagcggaggcggtggatcaggaggaggaggctcaggcggtggcggctcgcacatggacgtggtcatgactcagtccccgctgaccctgtcggtggcaattggacagagcgcatccatctcgtgcaagagctcacagtcgctgctggattccgacggaaagacttatctgaactggctgctccaaagaccagggcaatcaccgaaacgccttatctccctggtgtcgaaactcgactcgggtgtgccggatcggtttaccggtagcgggtccggcacggacttcactctccgcatttcgagggtggaagcggaggatctcgggatctactactgttggcagggaacccacttccctgggacttttggaggcggaactaagctggaaatcaag

SEQ ID NO: 99 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 100 - DNA, 336 bp, Homo sapiens
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 101 - protein, 14 aa, Artificial Sequence
  source: /note=“Description of Artificial Sequence: Synthetic peptide”
LEEKKGNYVVTDHC

SEQ ID NO: 102 - protein, 48 aa, Artificial Sequence
  source: /note=“Description of Artificial Sequence: Synthetic polypeptide”
QRRKYRSNKGESPVEPAEPCRYSCPREEEGSTIPIQEDYRKPEPACSP

SEQ ID NO: 103 - DNA, 123 bp, Artificial Sequence
  source: /note=“Description of Artificial Sequence: Synthetic polynucleotide”
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 104 - protein, 230 aa, Artificial Sequence
  source: /note=“Description of Artificial Sequence: Synthetic polypeptide”
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKM

SEQ ID NO: 105 - DNA, 690 bp, Artificial Sequence
  source: /note=“Description of Artificial Sequence: Synthetic polynucleotide”
gagagcaagtacggccctccctgccccccttgccctgcccccgagttcctgggcggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttcaatagcacctaccgggtggtgtccgtgctgaccgtgctgcaccaggactggctgaacggcaaggaatacaagtgtaaggtgtccaacaagggcctgcccagcagcatcgagaaaaccatcagcaaggccaagggccagcctcgggagccccaggtgtacaccctgccccctagccaagaggagatgaccaagaaccaggtgtccctgacctgcctggtgaagggcttctaccccagcgacatcgccgtggagtgggagagcaacggccagcccgagaacaactacaagaccaccccccctgtgctggacagcgacggcagcttcttcctgtacagccggctgaccgtggacaagagccggtggcaggagggcaacgtctttagctgctccgtgatgcacgaggccctgcacaaccactacacccagaagagcctgagcctgtccctgggcaagatg

SEQ ID NO: 106 - protein, 282 aa, Artificial Sequence
  source: /note=“Description of Artificial Sequence: Synthetic polypeptide”
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 107 - DNA, 847 bp, Artificial Sequence
  source: /note=“Description of Artificial Sequence: Synthetic polynucleotide”
aggtggcccgaaagtcccaaggcccaggcatctagtgttcctactgcacagccccaggcagaaggcagcctagccaaagctactactgcacctgccactacgcgcaatactggccgtggcggggaggagaagaaaaaggagaaagagaaagaagaacaggaagagagggagaccaagacccctgaatgtccatcccatacccagccgctgggcgtctatctcttgactcccgcagtacaggacttgtggcttagagataaggccacctttacatgtttcgtcgtgggctctgacctgaaggatgcccatttgacttgggaggttgccggaaaggtacccacagggggggttgaggaagggttgctggagcgccattccaatggctctcagagccagcactcaagactcacccttccgagatccctgtggaacgccgggacctctgtcacatgtactctaaatcatcctagcctgcccccacagcgtctgatggcccttagagagccagccgcccaggcaccagttaagcttagcctgaatctgctcgccagtagtgatcccccagaggccgccagctggctcttatgcgaagtgtccggctttagcccgcccaacatcttgctcatgtggctggaggaccagcgagaagtgaacaccagcggcttcgctccagcccggcccccaccccagccgggttctaccacattctgggcctggagtgtcttaagggtcccagcaccacctagcccccagccagccacatacacctgtgttgtgtcccatgaagatagcaggaccctgctaaatgcttctaggagtctggaggtttcctacgtgactgaccatt

SEQ ID NO: 108 - protein, 10 aa, Artificial Sequence
  source: /note=“Description of Artificial Sequence: Synthetic peptide”
GGGGSGGGGS

SEQ ID NO: 109 - DNA, 30 bp, Artificial Sequence
  source: /note=“Description of Artificial Sequence: Synthetic oligonucleotide”
ggtggcggaggttctggaggtggaggttcc

SEQ ID NO: 110 - protein, 30 aa, Artificial Sequence
  source: /note=“Description of Artificial Sequence: Synthetic polypeptide”
  SITE (1)..(30): /note=“This sequence may encompass 1-6 ′Gly Gly Gly Gly Ser′ repeating units”
  source: /note=“See specification as filed for detailed description of substitutions and preferred embodiments”
GGGGSGGGGSGGGGSGGGGSGGGGSGGGGS

SEQ ID NO: 111 - DNA, 150 bp, Artificial Sequence
  source: /note=“Description of Artificial Sequence: Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 112 - protein, 4 aa, Artificial Sequence
  source: /note=“Description of Artificial Sequence: Synthetic peptide”
GGGS

SEQ ID NO: 113 - protein, 20 aa, Artificial Sequence
  source: /note=“Description of Artificial Sequence: Synthetic peptide”
GGGGSGGGGSGGGGSGGGGS

SEQ ID NO: 114 - protein, 15 aa, Artificial Sequence
  source: /note=“Description of Artificial Sequence: Synthetic peptide”
GGGGSGGGGSGGGGS

SEQ ID NO: 115 - DNA, 5000 bp, Artificial Sequence
  source: /note=“Description of Artificial Sequence: Synthetic polynucleotide”
  misc_feature (1)..(5000): /note=“This sequence may encompass 50-5000 nucleotides”
  source: /note=“See specification as filed for detailed description of substitutions and preferred embodiments”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 116 - DNA, 2000 bp, Artificial Sequence
  source: /note=“Description of Artificial Sequence: Synthetic polynucleotide”
  misc_feature (1)..(2000): /note=“This sequence may encompass 50-2000 nucleotides”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 117 - DNA, 100 bp, Artificial Sequence
  source: /note=“Description of Artificial Sequence: Synthetic polynucleotide”
tttttttttttttttttttttttttttttttttttttttttttttttttttttttttttttttttttttttttttttttttttttttttttttttttttt

SEQ ID NO: 118 - DNA, 5000 bp, Artificial Sequence
  source: /note=“Description of Artificial Sequence: Synthetic polynucleotide”
  misc_feature (1)..(5000): /note=“This sequence may encompass 50-5000 nucleotides”
tttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttt

SEQ ID NO: 119 - DNA, 5000 bp, Artificial Sequence; identical to SEQ ID NO: 115
  source: /note=“Description of Artificial Sequence: Synthetic polynucleotide”
  misc_feature (1)..(5000): /note=“This sequence may encompass 100-5000 nucleotides”

SEQ ID NO: 120 - DNA, 400 bp, Artificial Sequence
  source: /note=“Description of Artificial Sequence: Synthetic polynucleotide”
  misc_feature (1)..(400): /note=“This sequence may encompass 100-400 nucleotides”
  source: /note=“See specification as filed for detailed description of substitutions and preferred embodiments”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 121 - protein, 487 aa, Artificial Sequence
  source: /note=“Description of Artificial Sequence: Synthetic polypeptide”
MVLLVTSLLLCELPHPAFLIIPDIQMTQSPSSLSASVGDRVIIICRASQGIRNNLAWYQQKPGKAPKRLIYAASNLQSGVPSRFTGSGSGTEFTLIVSSLQPEDFATYYCLQHHSYPLTSGGGTKVEIKFTGSTSGSGKPGSGEGSEVQVLESGGGLVQPGGSLRLSCAASGFTFSSYAMSWVRQAPGKGLEWVSAISGSGGSTNYADSVKGRFTISRDNSKNTLYLQMNSLRAEDTAVYYCAGSSGWSEYWGQGTLVTVSSASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 122 - protein, 114 aa, Mus musculus; identical to SEQ ID NO: 21

SEQ ID NO: 123 - protein, 114 aa, Artificial Sequence
  source: /note=“Description of Artificial Sequence: Synthetic polypeptide”
EIQLVQSGAEVKKPGATVKISCKGSGFNIEDYYIHWVQQAPGKGLEWMGRIDPENDETKYGPIFQGRVTITADTSTNTVYMELSSLRSEDTAVYYCAFRGGVYWGQGTTVTVSS

SEQ ID NO: 124 - protein, 114 aa, Artificial Sequence
  source: /note=“Description of Artificial Sequence: Synthetic polypeptide”
EIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSS

SEQ ID NO: 125 - protein, 112 aa, Mus musculus; identical to SEQ ID NO: 25

SEQ ID NO: 126 - protein, 112 aa, Artificial Sequence
  source: /note=“Description of Artificial Sequence: Synthetic polypeptide”
DVVMTQSPLSLPVTLGQPASISCKSSQSLLDSDGKTYLNWLQQRPGQSPRRLISLVSKLDSGVPDRFSGSGSGTDFTLKISRVEAEDVGVYYCWQGTHFPGTFGGGTKVEIK

SEQ ID NO: 127 - protein, 112 aa, Artificial Sequence
  source: /note=“Description of Artificial Sequence: Synthetic polypeptide”
DVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIK

SEQ ID NO: 128 - protein, 373 aa, Artificial Sequence
  source: /note=“Description of Artificial Sequence: Synthetic polypeptide”
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 129 - DNA, 1182 bp, Artificial Sequence
  source: /note=“Description of Artificial Sequence: Synthetic polynucleotide”
atggccctccctgtcactgccctgcttctccccctcgcactcctgctccacgccgctagaccacccggatggtttctggactctccggatcgcccgtggaatcccccaaccttctcaccggcactcttggttgtgactgagggcgataatgcgaccttcacgtgctcgttctccaacacctccgaatcattcgtgctgaactggtaccgcatgagcccgtcaaaccagaccgacaagctcgccgcgtttccggaagatcggtcgcaaccgggacaggattgtcggttccgcgtgactcaactgccgaatggcagagacttccacatgagcgtggtccgcgctaggcgaaacgactccgggacctacctgtgcggagccatctcgctggcgcctaaggcccaaatcaaagagagcttgagggccgaactgagagtgaccgagcgcagagctgaggtgccaactgcacatccatccccatcgcctcggcctgcggggcagtttcagaccctggtcacgaccactccggcgccgcgcccaccgactccggccccaactatcgcgagccagcccctgtcgctgaggccggaagcatgccgccctgccgccggaggtgctgtgcatacccggggattggacttcgcatgcgacatctacatttgggctcctctcgccggaacttgtggcgtgctccttctgtccctggtcatcaccctgtactgcaagcggggtcggaaaaagcttctgtacattttcaagcagcccttcatgaggcccgtgcaaaccacccaggaggaggacggttgctcctgccggttccccgaagaggaagaaggaggttgcgagctgcgcgtgaagttctcccggagcgccgacgcccccgcctataagcagggccagaaccagctgtacaacgaactgaacctgggacggcgggaagagtacgatgtgctggacaagcggcgcggccgggaccccgaaatgggcgggaagcctagaagaaagaaccctcaggaaggcctgtataacgagctgcagaaggacaagatggccgaggcctactccgaaattgggatgaagggagagcggcggaggggaaaggggcacgacggcctgtaccaaggactgtccaccgccaccaaggacacatacgatgccctgcacatgcaggcccttccccctcgc

SEQ ID NO: 130 - protein, 394 aa, Artificial Sequence
  source: /note=“Description of Artificial Sequence: Synthetic polypeptide”
MALPVTALLLPLALLLHAARPPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 131-134 - skipped (4 entries)

SEQ ID NO: 135 - protein, 1132 aa, Homo sapiens [sequence omitted]

SEQ ID NO: 136 - DNA, 4027 bp, Homo sapiens
caggcagcgtggtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgttgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcggggctgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactgatggccacccgcccacagccaggccgagagcagacaccagcagccctgtcacgccgggctctacgtcccagggagggaggggcggcccacacccaggcccgcaccgctgggagtctgaggcctgagtgagtgtttggccgaggcctgcatgtccggctgaaggctgagtgtccggctgaggcctgagcgagtgtccagccaagggctgagtgtccagcacacctgccgtcttcacttccccacaggctggcgctcggctccaccccagggccagcttttcctcaccaggagcccggcttccactccccacataggaatagtccatccccagattcgccattgttcacccctcgccctgccctcctttgccttccacccccaccatccaggtggagaccctgagaaggaccctgggagctctgggaatttggagtgaccaaaggtgtgccctgtacacaggcgaggaccctgcacctggatgggggtccctgtgggtcaaattggggggaggtgctgtgggagtaaaatactgaatatatgagtttttcagttttgaaaaaaaaaaaaaaaaaa

SEQ ID NO: 137 - protein, 5 aa, Artificial Sequence
  source: /note=“Description of Artificial Sequence: Synthetic peptide”
TYWMH

SEQ ID NO: 138 - protein, 17 aa, Artificial Sequence
  source: /note=“Description of Artificial Sequence: Synthetic peptide”
NIYPGTGGSNFDEKFKN

SEQ ID NO: 139 - protein, 8 aa, Artificial Sequence
  source: /note=“Description of Artificial Sequence: Synthetic peptide”
WTTGTGAY

SEQ ID NO: 140 - protein, 7 aa, Artificial Sequence
  source: /note=“Description of Artificial Sequence: Synthetic peptide”
GYTFTTY

SEQ ID NO: 141 - protein, 6 aa, Artificial Sequence
  source: /note=“Description of Artificial Sequence: Synthetic peptide”
YPGTGG

SEQ ID NO: 142 - protein, 117 aa, Artificial Sequence
  source: /note=“Description of Artificial Sequence: Synthetic polypeptide”
QVQLQQPGSELVRPGASVKLSCKASGYTFTTYWMHWVRQRPGQGLEWIGNIYPGTGGSNFDEKFKNRTSLTVDTSSTTAYMHLASLTSEDSAVYYCTRWTTGTGAYWGQGTLVTVSA

SEQ ID NO: 143 - DNA, 351 bp, Artificial Sequence
  source: /note=“Description of Artificial Sequence: Synthetic polynucleotide”
caggtccagctgcagcaacctgggtctgagctggtgaggcctggagcttcagtgaagctgtcctgcaaggcgtctggctacacattcaccacttactggatgcactgggtgaggcagaggcctggacaaggccttgagtggattggaaatatttatcctggtactggtggttctaacttcgatgagaagttcaaaaacaggacctcactgactgtagacacatcctccaccacagcctacatgcacctcgccagcctgacatctgaggactctgcggtctattactgtacaagatggactactgggacgggagcttattggggccaagggactctggtcactgtctctgca

SEQ ID NO: 144 - protein, 117 aa, Artificial Sequence
  source: /note=“Description of Artificial Sequence: Synthetic polypeptide”
QVQLQQSGSELVRPGASVKLSCKASGYTFTTYWMHWVRQRPGQGLEWIGNIYPGTGGSNFDEKFKNRTSLTVDTSSTTAYMHLASLTSEDSAVYYCTRWTTGTGAYWGQGTLVTVSA

SEQ ID NO: 145 - DNA, 351 bp, Artificial Sequence
  source: /note=“Description of Artificial Sequence: Synthetic polynucleotide”
caggtccagctgcagcagtctgggtctgagctggtgaggcctggagcttcagtgaagctgtcctgcaaggcgtctggctacacattcaccacttactggatgcactgggtgaggcagaggcctggacaaggccttgagtggattggaaatatttatcctggtactggtggttctaacttcgatgagaagttcaaaaacaggacctcactgactgtagacacatcctccaccacagcctacatgcacctcgccagcctgacatctgaggactctgcggtctattactgtacaagatggactactgggacgggagcttattggggccaagggactctggtcactgtctctgca

SEQ ID NO: 146 - protein, 17 aa, Artificial Sequence
  source: /note=“Description of Artificial Sequence: Synthetic peptide”
KSSQSLLDSGNQKNFLT

SEQ ID NO: 147 - protein, 7 aa, Artificial Sequence
  source: /note=“Description of Artificial Sequence: Synthetic peptide”
WASTRES

SEQ ID NO: 148 - protein, 9 aa, Artificial Sequence
  source: /note=“Description of Artificial Sequence: Synthetic peptide”
QNDYSYPCT

SEQ ID NO: 149 - protein, 13 aa, Artificial Sequence
  source: /note=“Description of Artificial Sequence: Synthetic peptide”
SQSLLDSGNQKNF

SEQ ID NO: 150 - protein, 3 aa, Artificial Sequence
  source: /note=“Description of Artificial Sequence: Synthetic peptide”
WAS

SEQ ID NO: 151 - protein, 6 aa, Artificial Sequence
  source: /note=“Description of Artificial Sequence: Synthetic peptide”
DYSYPC

SEQ ID NO: 152 - protein, 113 aa, Artificial Sequence
  source: /note=“Description of Artificial Sequence: Synthetic polypeptide”
DIVMTQSPSSLTVTAGEKVTMSCKSSQSLLDSGNQKNFLTWYQQKPGQPPKLLIFWASTRESGVPDRFTGSGSVTDFTLTISSVQAEDLAVYYCQNDYSYPCTFGGGTKLEIK

SEQ ID NO: 153 - DNA, 339 bp, Artificial Sequence
  source: /note=“Description of Artificial Sequence: Synthetic polynucleotide”
gacattgtgatgacccagtctccatcctccctgactgtgacagcaggagagaaggtcactatgagctgcaagtccagtcagagtctgttagacagtggaaatcaaaagaacttcttgacctggtaccagcagaaaccagggcagcctcctaaactgttgatcttctgggcatccactagggaatctggggtccctgatcgcttcacaggcagtggatctgtaacagatttcactctcaccatcagcagtgtgcaggctgaagacctggcagtttattactgtcagaatgattatagttatccgtgcacgttcggaggggggaccaagctggaaataaaa

SEQ ID NO: 154 - protein, 117 aa, Artificial Sequence
  source: /note=“Description of Artificial Sequence: Synthetic polypeptide”
QVQLQQPGSELVRPGASVKLSCKASGYTFTTYWMHWVRQRPGQGLEWIGNIYPGTGGSNFDEKFKNRTSLTVDTSSTTAYMHLASLTSEDSAVYYCTRWTTGTGAYWGQGTTVTVSS

SEQ ID NO: 155 - DNA, 351 bp, Artificial Sequence
  source: /note=“Description of Artificial Sequence: Synthetic polynucleotide”
caggtccagctgcagcagcctgggtctgagctggtgaggcctggagcttcagtgaagctgtcctgcaaggcgtctggctacacattcaccacttactggatgcactgggtgaggcagaggcctggacaaggccttgagtggattggaaatatttatcctggtactggtggttctaacttcgatgagaagttcaaaaacaggacctcactgactgtagacacatcctccaccacagcctacatgcacctcgccagcctgacatctgaggactctgcggtctattactgtacaagatggactactgggacgggagcttattggggccagggcaccaccgtgaccgtgtcctcc

SEQ ID NO: 156 - protein, 444 aa, Artificial Sequence
  source: /note=“Description of Artificial Sequence: Synthetic polypeptide”
QVQLQQPGSELVRPGASVKLSCKASGYTFTTYWMHWVRQRPGQGLEWIGNIYPGTGGSNFDEKFKNRTSLTVDTSSTTAYMHLASLTSEDSAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 157 - DNA, 1332 bp, Artificial Sequence
  source: /note=“Description of Artificial Sequence: Synthetic polynucleotide”
caggtccagctgcagcagcctgggtctgagctggtgaggcctggagcttcagtgaagctgtcctgcaaggcgtctggctacacattcaccacttactggatgcactgggtgaggcagaggcctggacaaggccttgagtggattggaaatatttatcctggtactggtggttctaacttcgatgagaagttcaaaaacaggacctcactgactgtagacacatcctccaccacagcctacatgcacctcgccagcctgacatctgaggactctgcggtctattactgtacaagatggactactgggacgggagcttattggggccagggcaccaccgtgaccgtgtcctccgcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtgtccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 158 - protein, 117 aa, Artificial Sequence
  source: /note=“Description of Artificial Sequence: Synthetic polypeptide”
QVQLQQSGSELVRPGASVKLSCKASGYTFTTYWMHWVRQRPGQGLEWIGNIYPGTGGSNFDEKFKNRTSLTVDTSSTTAYMHLASLTSEDSAVYYCTRWTTGTGAYWGQGTTVTVSS

SEQ ID NO: 159 - DNA, 351 bp, Artificial Sequence
  source: /note=“Description of Artificial Sequence: Synthetic polynucleotide”
caggtccagctgcagcagtctgggtctgagctggtgaggcctggagcttcagtgaagctgtcctgcaaggcgtctggctacacattcaccacttactggatgcactgggtgaggcagaggcctggacaaggccttgagtggattggaaatatttatcctggtactggtggttctaacttcgatgagaagttcaaaaacaggacctcactgactgtagacacatcctccaccacagcctacatgcacctcgccagcctgacatctgaggactctgcggtctattactgtacaagatggactactgggacgggagcttattggggccagggcaccaccgtgaccgtgtcctcc

SEQ ID NO: 160 - protein, 444 aa, Artificial Sequence
  source: /note=“Description of Artificial Sequence: Synthetic polypeptide”
QVQLQQSGSELVRPGASVKLSCKASGYTFTTYWMHWVRQRPGQGLEWIGNIYPGTGGSNFDEKFKNRTSLTVDTSSTTAYMHLASLTSEDSAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 161 - DNA, 1332 bp, Artificial Sequence
  source: /note=“Description of Artificial Sequence: Synthetic polynucleotide”
caggtccagctgcagcagtctgggtctgagctggtgaggcctggagcttcagtgaagctgtcctgcaaggcgtctggctacacattcaccacttactggatgcactgggtgaggcagaggcctggacaaggccttgagtggattggaaatatttatcctggtactggtggttctaacttcgatgagaagttcaaaaacaggacctcactgactgtagacacatcctccaccacagcctacatgcacctcgccagcctgacatctgaggactctgcggtctattactgtacaagatggactactgggacgggagcttattggggccagggcaccaccgtgaccgtgtcctccgcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtgtccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 162 - protein, 113 aa, Artificial Sequence
  source: /note=“Description of Artificial Sequence: Synthetic polypeptide”
DIVMTQSPSSLTVTAGEKVTMSCKSSQSLLDSGNQKNFLTWYQQKPGQPPKLLIFWASTRESGVPDRFTGSGSVTDFTLTISSVQAEDLAVYYCQNDYSYPCTFGQGTKVEIK

SEQ ID NO: 163 - DNA, 339 bp, Artificial Sequence
  source: /note=“Description of Artificial Sequence: Synthetic polynucleotide”
gacattgtgatgacccagtctccatcctccctgactgtgacagcaggagagaaggtcactatgagctgcaagtccagtcagagtctgttagacagtggaaatcaaaagaacttcttgacctggtaccagcagaaaccagggcagcctcctaaactgttgatcttctgggcatccactagggaatctggggtccctgatcgcttcacaggcagtggatctgtaacagatttcactctcaccatcagcagtgtgcaggctgaagacctggcagtttattactgtcagaatgattatagttatccgtgcacgttcggccaagggaccaaggtggaaatcaaa

SEQ ID NO: 164 - protein, 220 aa, Artificial Sequence
  source: /note=“Description of Artificial Sequence: Synthetic polypeptide”
DIVMTQSPSSLTVTAGEKVTMSCKSSQSLLDSGNQKNFLTWYQQKPGQPPKLLIFWASTRESGVPDRFTGSGSVTDFTLTISSVQAEDLAVYYCQNDYSYPCTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 165 - DNA, 660 bp, Artificial Sequence
  source: /note=“Description of Artificial Sequence: Synthetic polynucleotide”
gacattgtgatgacccagtctccatcctccctgactgtgacagcaggagagaaggtcactatgagctgcaagtccagtcagagtctgttagacagtggaaatcaaaagaacttcttgacctggtaccagcagaaaccagggcagcctcctaaactgttgatcttctgggcatccactagggaatctggggtccctgatcgcttcacaggcagtggatctgtaacagatttcactctcaccatcagcagtgtgcaggctgaagacctggcagtttattactgtcagaatgattatagttatccgtgcacgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 166 - protein, 9 aa, Artificial Sequence
  source: /note=“Description of Artificial Sequence: Synthetic peptide”
QNDYSYPYT

SEQ ID NO: 167 - protein, 6 aa, Artificial Sequence
  source: /note=“Description of Artificial Sequence: Synthetic peptide”
DYSYPY

SEQ ID NO: 168 - protein, 113 aa, Artificial Sequence
  source: /note=“Description of Artificial Sequence: Synthetic polypeptide”
DIVMTQSPSSLTVTAGEKVTMSCKSSQSLLDSGNQKNFLTWYQQKPGQPPKLLIFWASTRESGVPDRFTGSGSVTDFTLTISSVQAEDLAVYYCQNDYSYPYTFGQGTKVEIK

SEQ ID NO: 169 - DNA, 339 bp, Artificial Sequence
  source: /note=“Description of Artificial Sequence: Synthetic polynucleotide”
gacattgtgatgacccagtctccatcctccctgactgtgacagcaggagagaaggtcactatgagctgcaagtccagtcagagtctgttagacagtggaaatcaaaagaacttcttgacctggtaccagcagaaaccagggcagcctcctaaactgttgatcttctgggcatccactagggaatctggggtccctgatcgcttcacaggcagtggatctgtaacagatttcactctcaccatcagcagtgtgcaggctgaagacctggcagtttattactgtcagaatgattatagttatccgtacacgttcggccaagggaccaaggtggaaatcaaa

SEQ ID NO: 170 - protein, 220 aa, Artificial Sequence
  source: /note=“Description of Artificial Sequence: Synthetic polypeptide”
DIVMTQSPSSLTVTAGEKVTMSCKSSQSLLDSGNQKNFLTWYQQKPGQPPKLLIFWASTRESGVPDRFTGSGSVTDFTLTISSVQAEDLAVYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 171 - DNA, 660 bp, Artificial Sequence
  source: /note=“Description of Artificial Sequence: Synthetic polynucleotide”
gacattgtgatgacccagtctccatcctccctgactgtgacagcaggagagaaggtcactatgagctgcaagtccagtcagagtctgttagacagtggaaatcaaaagaacttcttgacctggtaccagcagaaaccagggcagcctcctaaactgttgatcttctgggcatccactagggaatctggggtccctgatcgcttcacaggcagtggatctgtaacagatttcactctcaccatcagcagtgtgcaggctgaagacctggcagtttattactgtcagaatgattatagttatccgtacacgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 172 - protein, 117 aa, Artificial Sequence
  source: /note=“Description of Artificial Sequence: Synthetic polypeptide”
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSS

SEQ ID NO: 173 - DNA, 351 bp, Artificial Sequence
  source: /note=“Description of Artificial Sequence: Synthetic polynucleotide”
gaagtgcagctggtgcagtctggagcagaggtgaaaaagcccggggagtctctgaggatctcctgtaagggttctggctacacattcaccacttactggatgcactgggtgcgacaggccactggacaagggcttgagtggatgggtaatatttatcctggtactggtggttctaacttcgatgagaagttcaagaacagagtcacgattaccgcggacaaatccacgagcacagcctacatggagctgagcagcctgagatctgaggacacggccgtgtattactgtacaagatggactactgggacgggagcttattggggccagggcaccaccgtgaccgtgtcctcc

SEQ ID NO: 174 - protein, 444 aa, Artificial Sequence
  source: /note=“Description of Artificial Sequence: Synthetic polypeptide”
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 175 - DNA, 1332 bp, Artificial Sequence
  source: /note=“Description of Artificial Sequence: Synthetic polynucleotide”
gaagtgcagctggtgcagtctggagcagaggtgaaaaagcccggggagtctctgaggatctcctgtaagggttctggctacacattcaccacttactggatgcactgggtgcgacaggccactggacaagggcttgagtggatgggtaatatttatcctggtactggtggttctaacttcgatgagaagttcaagaacagagtcacgattaccgcggacaaatccacgagcacagcctacatggagctgagcagcctgagatctgaggacacggccgtgtattactgtacaagatggactactgggacgggagcttattggggccagggcaccaccgtgaccgtgtcctccgcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtgtccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 176 - protein, 113 aa, Artificial Sequence
  source: /note=“Description of Artificial Sequence: Synthetic polypeptide”
EIVLTQSPATLSLSPGERATLSCKSSQSLLDSGNQKNFLTWYQQKPGQAPRLLIYWASTRESGVPSRFSGSGSGTEFTLTISSLQPDDFATYYCQNDYSYPYTFGQGTKVEIK

SEQ ID NO: 177 - DNA, 339 bp, Artificial Sequence
  source: /note=“Description of Artificial Sequence: Synthetic polynucleotide”
gaaattgtgttgacacagtctccagccaccctgtctttgtctccaggggaaagagccaccctctcctgcaagtccagtcagagtctgttagacagtggaaatcaaaagaacttcttgacctggtaccagcagaaacctggccaggctcccaggctcctcatctattgggcatccactagggaatctggggtcccatcaaggttcagcggcagtggatctgggacagaattcactctcaccatcagcagcctgcagcctgatgattttgcaacttattactgtcagaatgattatagttatccgtacacgttcggccaagggaccaaggtggaaatcaaa

SEQ ID NO: 178 - protein, 220 aa, Artificial Sequence
  source: /note=“Description of Artificial Sequence: Synthetic polypeptide”
EIVLTQSPATLSLSPGERATLSCKSSQSLLDSGNQKNFLTWYQQKPGQAPRLLIYWASTRESGVPSRFSGSGSGTEFTLTISSLQPDDFATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 179 - DNA, 660 bp, Artificial Sequence
  source: /note=“Description of Artificial Sequence: Synthetic polynucleotide”
gaaattgtgttgacacagtctccagccaccctgtctttgtctccaggggaaagagccaccctctcctgcaagtccagtcagagtctgttagacagtggaaatcaaaagaacttcttgacctggtaccagcagaaacctggccaggctcccaggctcctcatctattgggcatccactagggaatctggggtcccatcaaggttcagcggcagtggatctgggacagaattcactctcaccatcagcagcctgcagcctgatgattttgcaacttattactgtcagaatgattatagttatccgtacacgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 180 - protein, 113 aa, Artificial Sequence
  source: /note=“Description of Artificial Sequence: Synthetic polypeptide”
DIQMTQSPSSLSASVGDRVTITCKSSQSLLDSGNQKNFLTWYQQKPGQAPRLLIYWASTRESGIPPRFSGSGYGTDFTLTINNIESEDAAYYFCQNDYSYPYTFGQGTKVEIK

SEQ ID NO: 181 - DNA, 339 bp, Artificial Sequence
  source: /note=“Description of Artificial Sequence: Synthetic polynucleotide”
gacatccagatgacccagtctccatcctccctgtctgcatctgtaggagacagagtcaccatcacttgcaagtccagtcagagtctgttagacagtggaaatcaaaagaacttcttgacctggtaccagcagaaacctggccaggctcccaggctcctcatctattgggcatccactagggaatctgggatcccacctcgattcagtggcagcgggtatggaacagattttaccctcacaattaataacatagaatctgaggatgctgcatattacttctgtcagaatgattatagttatccgtacacgttcggccaagggaccaaggtggaaatcaaa

SEQ ID NO: 182 - protein, 220 aa, Artificial Sequence
  source: /note=“Description of Artificial Sequence: Synthetic polypeptide”
DIQMTQSPSSLSASVGDRVTITCKSSQSLLDSGNQKNFLTWYQQKPGQAPRLLIYWASTRESGIPPRFSGSGYGTDFTLTINNIESEDAAYYFCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 183 - DNA, 660 bp, Artificial Sequence
  source: /note=“Description of Artificial Sequence: Synthetic polynucleotide”
gacatccagatgacccagtctccatcctccctgtctgcatctgtaggagacagagtcaccatcacttgcaagtccagtcagagtctgttagacagtggaaatcaaaagaacttcttgacctggtaccagcagaaacctggccaggctcccaggctcctcatctattgggcatccactagggaatctgggatcccacctcgattcagtggcagcgggtatggaacagattttaccctcacaattaataacatagaatctgaggatgctgcatattacttctgtcagaatgattatagttatccgtacacgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 184 - protein, 117 aa, Artificial Sequence
  source: /note=“Description of Artificial Sequence: Synthetic polypeptide”
EVQLVQSGAEVKKPGESLRISCKGSGYTFTTYWMHWIRQSPSRGLEWLGNIYPGTGGSNFDEKFKNRFTISRDNSKNTLYLQMNSLRAEDTAVYYCTRWTTGTGAYWGQGTTVTVSS

SEQ ID NO: 185 - DNA, 351 bp, Artificial Sequence
  source: /note=“Description of Artificial Sequence: Synthetic polynucleotide”
gaagtgcagctggtgcagtctggagcagaggtgaaaaagcccggggagtctctgaggatctcctgtaagggttctggctacacattcaccacttactggatgcactggatcaggcagtccccatcgagaggccttgagtggctgggtaatatttatcctggtactggtggttctaacttcgatgagaagttcaagaacagattcaccatctccagagacaattccaagaacacgctgtatcttcaaatgaacagcctgagagccgaggacacggccgtgtattactgtacaagatggactactgggacgggagcttattggggccagggcaccaccgtgaccgtgtcctcc

SEQ ID NO: 186 - protein, 444 aa, Artificial Sequence
  source: /note=“Description of Artificial Sequence: Synthetic polypeptide”
EVQLVQSGAEVKKPGESLRISCKGSGYTFTTYWMHWIRQSPSRGLEWLGNIYPGTGGSNFDEKFKNRFTISRDNSKNTLYLQMNSLRAEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 187 - DNA, 1332 bp, Artificial Sequence
  source: /note=“Description of Artificial Sequence: Synthetic polynucleotide”
gaagtgcagctggtgcagtctggagcagaggtgaaaaagcccggggagtctctgaggatctcctgtaagggttctggctacacattcaccacttactggatgcactggatcaggcagtccccatcgagaggccttgagtggctgggtaatatttatcctggtactggtggttctaacttcgatgagaagttcaagaacagattcaccatctccagagacaattccaagaacacgctgtatcttcaaatgaacagcctgagagccgaggacacggccgtgtattactgtacaagatggactactgggacgggagcttattggggccagggcaccaccgtgaccgtgtcctccgcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtgtccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 188 - protein, 113 aa, Artificial Sequence
  source: /note=“Description of Artificial Sequence: Synthetic polypeptide”
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIK

SEQ ID NO: 189 - DNA, 339 bp, Artificial Sequence
  source: /note=“Description of Artificial Sequence: Synthetic polynucleotide”
gaaattgtgttgacacagtctccagccaccctgtctttgtctccaggggaaagagccaccctctcctgcaagtccagtcagagtctgttagacagtggaaatcaaaagaacttcttgacctggtatcagcagaaaccagggaaagctcctaagctcctgatctattgggcatccactagggaatctggggtcccatcaaggttcagtggaagtggatctgggacagattttactttcaccatcagcagcctgcagcctgaagatattgcaacatattactgtcagaatgattatagttatccgtacacgttcggccaagggaccaaggtggaaatcaaa

SEQ ID NO: 190 - protein, 220 aa, Artificial Sequence
  source: /note=“Description of Artificial Sequence: Synthetic polypeptide”
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 191 - DNA, 660 bp, Artificial Sequence
  source: /note=“Description of Artificial Sequence: Synthetic polynucleotide”
gaaattgtgttgacacagtctccagccaccctgtctttgtctccaggggaaagagccaccctctcctgcaagtccagtcagagtctgttagacagtggaaatcaaaagaacttcttgacctggtatcagcagaaaccagggaaagctcctaagctcctgatctattgggcatccactagggaatctggggtcccatcaaggttcagtggaagtggatctgggacagattttactttcaccatcagcagcctgcagcctgaagatattgcaacatattactgtcagaatgattatagttatccgtacacgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 192 - protein, 113 aa, Artificial Sequence
  source: /note=“Description of Artificial Sequence: Synthetic polypeptide”
DIVMTQTPLSLPVTPGEPASISCKSSQSLLDSGNQKNFLTWYQQKPGQAPRLLIYWASTRESGVPSRFSGSGSGTDFTFTISSLEAEDAATYYCQNDYSYPYTFGQGTKVEIK

SEQ ID NO: 193 - DNA, 339 bp, Artificial Sequence
  source: /note=“Description of Artificial Sequence: Synthetic polynucleotide”
gatattgtgatgacccagactccactctccctgcccgtcacccctggagagccggcctccatctcctgcaagtccagtcagagtctgttagacagtggaaatcaaaagaacttcttgacctggtaccagcagaaacctggccaggctcccaggctcctcatctattgggcatccactagggaatctggggtcccctcgaggttcagtggcagtggatctgggacagatttcacctttaccatcagtagcctggaagctgaagatgctgcaacatattactgtcagaatgattatagttatccgtacacgttcggccaagggaccaaggtggaaatcaaa

SEQ ID NO: 194 - protein, 220 aa, Artificial Sequence
  source: /note=“Description of Artificial Sequence: Synthetic polypeptide”
DIVMTQTPLSLPVTPGEPASISCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 195 - DNA, 660 bp, Artificial Sequence
  source: /note=“Description of Artificial Sequence: Synthetic polynucleotide”
gatattgtgatgacccagactccactctccctgcccgtcacccctggagagccggcctccatctcctgcaagtccagtcagagtctgttagacagtggaaatcaaaagaacttcttgacctggtaccagcagaaacctggccaggctcccaggctcctcatctattgggcatccactagggaatctggggtcccctcgaggttcagtggcagtggatctgggacagatttcacctttaccatcagtagcctggaagctgaagatgctgcaacatattactgtcagaatgattatagttatccgtacacgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 196 - protein, 113 aa, Artificial Sequence
  source: /note=“Description of Artificial Sequence: Synthetic polypeptide”
EIVLTQSPATLSLSPGERATLSCKSSQSLLDSGNQKNFLTWYQQKPGKAPKLLIYWASTRESGVPSRFSGSGSGTDFTFTISSLEAEDAATYYCQNDYSYPYTFGQGTKVEIK

SEQ ID NO: 197 - DNA, 339 bp, Artificial Sequence
  source: /note=“Description of Artificial Sequence: Synthetic polynucleotide”
gaaattgtgttgacacagtctccagccaccctgtctttgtctccaggggaaagagccaccctctcctgcaagtccagtcagagtctgttagacagtggaaatcaaaagaacttcttgacctggtatcagcagaaaccagggaaagctcctaagctcctgatctattgggcatccactagggaatctggggtcccctcgaggttcagtggcagtggatctgggacagatttcacctttaccatcagtagcctggaagctgaagatgctgcaacatattactgtcagaatgattatagttatccgtacacgttcggccaagggaccaaggtggaaatcaaa

SEQ ID NO: 198 - protein, 220 aa, Artificial Sequence
  source: /note=“Description of Artificial Sequence: Synthetic polypeptide”
EIVLTQSPATLSLSPGERATLSCKSSQSLLDSGNQKNFLTWYQQKPGKAPKLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 199 - DNA, 660 bp, Artificial Sequence
  source: /note=“Description of Artificial Sequence: Synthetic polynucleotide”
gaaattgtgttgacacagtctccagccaccctgtctttgtctccaggggaaagagccaccctctcctgcaagtccagtcagagtctgttagacagtggaaatcaaaagaacttcttgacctggtatcagcagaaaccagggaaagctcctaagctcctgatctattgggcatccactagggaatctggggtcccctcgaggttcagtggcagtggatctgggacagatttcacctttaccatcagtagcctggaagctgaagatgctgcaacatattactgtcagaatgattatagttatccgtacacgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 200 - protein, 113 aa, Artificial Sequence
  source: /note=“Description of Artificial Sequence: Synthetic polypeptide”
EIVLTQSPDFQSVTPKEKVTITCKSSQSLLDSGNQKNFLTWYQQKPGQAPRLLIYWASTRESGVPSRFSGSGSGTDFTFTISSLEAEDAATYYCQNDYSYPYTFGQGTKVEIK

SEQ ID NO: 201 - DNA, 339 bp, Artificial Sequence
  source: /note=“Description of Artificial Sequence: Synthetic polynucleotide”
gaaattgtgctgactcagtctccagactttcagtctgtgactccaaaggagaaagtcaccatcacctgcaagtccagtcagagtctgttagacagtggaaatcaaaagaacttcttgacctggtaccagcagaaacctggccaggctcccaggctcctcatctattgggcatccactagggaatctggggtcccctcgaggttcagtggcagtggatctgggacagatttcacctttaccatcagtagcctggaagctgaagatgctgcaacatattactgtcagaatgattatagttatccgtacacgttcggccaagggaccaaggtggaaatcaaa

SEQ ID NO: 202 - protein, 220 aa, Artificial Sequence
  source: /note=“Description of Artificial Sequence: Synthetic polypeptide”
EIVLTQSPDFQSVTPKEKVTITCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 203 - DNA, 660 bp, Artificial Sequence
  source: /note=“Description of Artificial Sequence: Synthetic polynucleotide”
gaaattgtgctgactcagtctccagactttcagtctgtgactccaaaggagaaagtcaccatcacctgcaagtccagtcagagtctgttagacagtggaaatcaaaagaacttcttgacctggtaccagcagaaacctggccaggctcccaggctcctcatctattgggcatccactagggaatctggggtcccctcgaggttcagtggcagtggatctgggacagatttcacctttaccatcagtagcctggaagctgaagatgctgcaacatattactgtcagaatgattatagttatccgtacacgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 204 - protein, 113 aa, Artificial Sequence
  source: /note=“Description of Artificial Sequence: Synthetic polypeptide”
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIK

SEQ ID NO: 205 - DNA, 339 bp, Artificial Sequence
  source: /note=“Description of Artificial Sequence: Synthetic polynucleotide”
gaaattgtgttgacacagtctccagccaccctgtctttgtctccaggggaaagagccaccctctcctgcaagtccagtcagagtctgttagacagtggaaatcaaaagaacttcttgacctggtaccagcagaaacctggccaggctcccaggctcctcatctattgggcatccactagggaatctggggtcccctcgaggttcagtggcagtggatctgggacagatttcacctttaccatcagtagcctggaagctgaagatgctgcaacatattactgtcagaatgattatagttatccgtacacgttcggccaagggaccaaggtggaaatcaaa

SEQ ID NO: 206 - protein, 220 aa, Artificial Sequence
  source: /note=“Description of Artificial Sequence: Synthetic polypeptide”
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 207 - DNA, 660 bp, Artificial Sequence
  source: /note=“Description of Artificial Sequence: Synthetic polynucleotide”
gaaattgtgttgacacagtctccagccaccctgtctttgtctccaggggaaagagccaccctctcctgcaagtccagtcagagtctgttagacagtggaaatcaaaagaacttcttgacctggtaccagcagaaacctggccaggctcccaggctcctcatctattgggcatccactagggaatctggggtcccctcgaggttcagtggcagtggatctgggacagatttcacctttaccatcagtagcctggaagctgaagatgctgcaacatattactgtcagaatgattatagttatccgtacacgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 208 - protein, 113 aa, Artificial Sequence
  source: /note=“Description of Artificial Sequence: Synthetic polypeptide”
DIQMTQSPSSLSASVGDRVTITCKSSQSLLDSGNQKNFLTWYLQKPGQSPQLLIYWASTRESGVPSRFSGSGSGTDFTFTISSLEAEDAATYYCQNDYSYPYTFGQGTKVEIK

SEQ ID NO: 209 - DNA, 339 bp, Artificial Sequence
  source: /note=“Description of Artificial Sequence: Synthetic polynucleotide”
gacatccagatgacccagtctccatcctccctgtctgcatctgtaggagacagagtcaccatcacttgcaagtccagtcagagtctgttagacagtggaaatcaaaagaacttcttgacctggtacctgcagaagccagggcagtctccacagctcctgatctattgggcatccactagggaatctggggtcccctcgaggttcagtggcagtggatctgggacagatttcacctttaccatcagtagcctggaagctgaagatgctgcaacatattactgtcagaatgattatagttatccgtacacgttcggccaagggaccaaggtggaaatcaaa

SEQ ID NO: 210 - protein, 220 aa, Artificial Sequence
  source: /note=“Description of Artificial Sequence: Synthetic polypeptide”
DIQMTQSPSSLSASVGDRVTITCKSSQSLLDSGNQKNFLTWYLQKPGQSPQLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 211 - DNA, 660 bp, Artificial Sequence
  source: /note=“Description of Artificial Sequence: Synthetic polynucleotide”
gacatccagatgacccagtctccatcctccctgtctgcatctgtaggagacagagtcaccatcacttgcaagtccagtcagagtctgttagacagtggaaatcaaaagaacttcttgacctggtacctgcagaagccagggcagtctccacagctcctgatctattgggcatccactagggaatctggggtcccctcgaggttcagtggcagtggatctgggacagatttcacctttaccatcagtagcctggaagctgaagatgctgcaacatattactgtcagaatgattatagttatccgtacacgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 212 - protein, 113 aa, Artificial Sequence
  source: /note=“Description of Artificial Sequence: Synthetic polypeptide”
DVVMTQSPLSLPVTLGQPASISCKSSQSLLDSGNQKNFLTWYQQKPGKAPKLLIYWASTRESGVPSRFSGSGSGTDFTFTISSLEAEDAATYYCQNDYSYPYTFGQGTKVEIK

SEQ ID NO: 213 - DNA, 339 bp, Artificial Sequence
  source: /note=“Description of Artificial Sequence: Synthetic polynucleotide”
gatgttgtgatgactcagtctccactctccctgcccgtcacccttggacagccggcctccatctcctgcaagtccagtcagagtctgttagacagtggaaatcaaaagaacttcttaacctggtatcagcagaaaccagggaaagctcctaagctcctgatctattgggcatccactagggaatctggggtcccctcgaggttcagtggcagtggatctgggacagatttcacctttaccatcagtagcctggaagctgaagatgctgcaacatattactgtcagaatgattatagttatccgtacacgttcggccaagggaccaaggtggaaatcaaa

SEQ ID NO: 214 - protein, 220 aa, Artificial Sequence
  source: /note=“Description of Artificial Sequence: Synthetic polypeptide”
DVVMTQSPLSLPVTLGQPASISCKSSQSLLDSGNQKNFLTWYQQKPGKAPKLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 215 - DNA, 660 bp, Artificial Sequence
  source: /note=“Description of Artificial Sequence: Synthetic polynucleotide”
gatgttgtgatgactcagtctccactctccctgcccgtcacccttggacagccggcctccatctcctgcaagtccagtcagagtctgttagacagtggaaatcaaaagaacttcttaacctggtatcagcagaaaccagggaaagctcctaagctcctgatctattgggcatccactagggaatctggggtcccctcgaggttcagtggcagtggatctgggacagatttcacctttaccatcagtagcctggaagctgaagatgctgcaacatattactgtcagaatgattatagttatccgtacacgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 216 - protein, 117 aa, Artificial Sequence
  source: /note=“Description of Artificial Sequence: Synthetic polypeptide”
QVQLVQSGAEVKKPGASVKVSCKASGYTFTTYWMHWIRQSPSRGLEWLGNIYPGTGGSNFDEKFKNRFTISRDNSKNTLYLQMNSLRAEDTAVYYCTRWTTGTGAYWGQGTTVTVSS

SEQ ID NO: 217 - DNA, 351 bp, Artificial Sequence
  source: /note=“Description of Artificial Sequence: Synthetic polynucleotide”
caggttcagctggtgcagtctggagctgaggtgaagaagcctggggcctcagtgaaggtctcctgcaaggcttctggctacacattcaccacttactggatgcactggatcaggcagtccccatcgagaggccttgagtggctgggtaatatttatcctggtactggtggttctaacttcgatgagaagttcaagaacagattcaccatctccagagacaattccaagaacacgctgtatcttcaaatgaacagcctgagagccgaggacacggccgtgtattactgtacaagatggactactgggacgggagcttactggggccagggcaccaccgtgaccgtgtcctcc

SEQ ID NO: 218 - protein, 444 aa, Artificial Sequence
  source: /note=“Description of Artificial Sequence: Synthetic polypeptide”
QVQLVQSGAEVKKPGASVKVSCKASGYTFTTYWMHWIRQSPSRGLEWLGNIYPGTGGSNFDEKFKNRFTISRDNSKNTLYLQMNSLRAEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 219 - DNA, 1332 bp, Artificial Sequence
  source: /note=“Description of Artificial Sequence: Synthetic polynucleotide”
caggttcagctggtgcagtctggagctgaggtgaagaagcctggggcctcagtgaaggtctcctgcaaggcttctggctacacattcaccacttactggatgcactggatcaggcagtccccatcgagaggccttgagtggctgggtaatatttatcctggtactggtggttctaacttcgatgagaagttcaagaacagattcaccatctccagagacaattccaagaacacgctgtatcttcaaatgaacagcctgagagccgaggacacggccgtgtattactgtacaagatggactactgggacgggagcttactggggccagggcaccaccgtgaccgtgtcctccgcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtgtccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 220 - protein, 117 aa, Artificial Sequence
  source: /note=“Description of Artificial Sequence: Synthetic polypeptide”
EVQLVQSGAEVKKPGESLRISCKGSGYTFTTYWMHWVRQAPGQGLEWMGNIYPGTGGSNFDEKFKNRFTISRDNSKNTLYLQMNSLRAEDTAVYYCTRWTTGTGAYWGQGTTVTVSS

SEQ ID NO: 221 - DNA, 351 bp, Artificial Sequence
  source: /note=“Description of Artificial Sequence: Synthetic polynucleotide”
gaagtgcagctggtgcagtctggagcagaggtgaaaaagcccggggagtctctgaggatctcctgtaagggttctggctacacattcaccacttactggatgcactgggtgcgacaggcccctggacaagggcttgagtggatgggtaatatttatcctggtactggtggttctaacttcgatgagaagttcaagaacagattcaccatctccagagacaattccaagaacacgctgtatcttcaaatgaacagcctgagagccgaggacacggccgtgtattactgtacaagatggactactgggacgggagcttattggggccagggcaccaccgtgaccgtgtcctcc

SEQ ID NO: 222 - protein, 444 aa, Artificial Sequence
  source: /note=“Description of Artificial Sequence: Synthetic polypeptide”
EVQLVQSGAEVKKPGESLRISCKGSGYTFTTYWMHWVRQAPGQGLEWMGNIYPGTGGSNFDEKFKNRFTISRDNSKNTLYLQMNSLRAEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 223 - DNA, 1332 bp, Artificial Sequence
  source: /note=“Description of Artificial Sequence: Synthetic polynucleotide”
gaagtgcagctggtgcagtctggagcagaggtgaaaaagcccggggagtctctgaggatctcctgtaagggttctggctacacattcaccacttactggatgcactgggtgcgacaggcccctggacaagggcttgagtggatgggtaatatttatcctggtactggtggttctaacttcgatgagaagttcaagaacagattcaccatctccagagacaattccaagaacacgctgtatcttcaaatgaacagcctgagagccgaggacacggccgtgtattactgtacaagatggactactgggacgggagcttattggggccagggcaccaccgtgaccgtgtcctccgcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtgtccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 224 - DNA, 351 bp, Artificial Sequence
  source: /note=“Description of Artificial Sequence: Synthetic polynucleotide”
gaagtgcagctggtgcagtctggcgccgaagtgaagaagcctggcgagtccctgcggatctcctgcaagggctctggctacaccttcaccacctactggatgcactgggtgcgacaggctaccggccagggcctggaatggatgggcaacatctatcctggcaccggcggctccaacttcgacgagaagttcaagaacagagtgaccatcaccgccgacaagtccacctccaccgcctacatggaactgtcctccctgagatccgaggacaccgccgtgtactactgcacccggtggacaaccggcacaggcgcttattggggccagggcaccacagtgaccgtgtcctct

SEQ ID NO: 225 - protein, 443 aa, Artificial Sequence
  source: /note=“Description of Artificial Sequence: Synthetic polypeptide”
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 226 - DNA, 1329 bp, Artificial Sequence
  source: /note=“Description of Artificial Sequence: Synthetic polynucleotide”
gaagtgcagctggtgcagtctggcgccgaagtgaagaagcctggcgagtccctgcggatctcctgcaagggctctggctacaccttcaccacctactggatgcactgggtgcgacaggctaccggccagggcctggaatggatgggcaacatctatcctggcaccggcggctccaacttcgacgagaagttcaagaacagagtgaccatcaccgccgacaagtccacctccaccgcctacatggaactgtcctccctgagatccgaggacaccgccgtgtactactgcacccggtggacaaccggcacaggcgcttattggggccagggcaccacagtgaccgtgtcctctgcttctaccaaggggcccagcgtgttccccctggccccctgctccagaagcaccagcgagagcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcaccaagacctacacctgtaacgtggaccacaagcccagcaacaccaaggtggacaagagggtggagagcaagtacggcccaccctgccccccctgcccagcccccgagttcctgggcggacccagcgtgttcctgttcccccccaagcccaaggacaccctgatgatcagcagaacccccgaggtgacctgtgtggtggtggacgtgtcccaggaggaccccgaggtccagttcaactggtacgtggacggcgtggaggtgcacaacgccaagaccaagcccagagaggagcagtttaacagcacctaccgggtggtgtccgtgctgaccgtgctgcaccaggactggctgaacggcaaagagtacaagtgtaaggtctccaacaagggcctgccaagcagcatcgaaaagaccatcagcaaggccaagggccagcctagagagccccaggtctacaccctgccacccagccaagaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaggctgaccgtggacaagtccagatggcaggagggcaacgtctttagctgctccgtgatgcacgaggccctgcacaaccactacacccagaagagcctgagcctgtccctgggc

SEQ ID NO: 227 - DNA, 339 bp, Artificial Sequence
  source: /note=“Description of Artificial Sequence: Synthetic polynucleotide”
gagatcgtgctgacccagtcccctgccaccctgtcactgtctccaggcgagagagctaccctgtcctgcaagtcctcccagtccctgctggactccggcaaccagaagaacttcctgacctggtatcagcagaagcccggccaggcccccagactgctgatctactgggcctccacccgggaatctggcgtgccctctagattctccggctccggctctggcaccgagtttaccctgaccatctccagcctgcagcccgacgacttcgccacctactactgccagaacgactactcctacccctacaccttcggccagggcaccaaggtggaaatcaag

SEQ ID NO: 228 - DNA, 660 bp, Artificial Sequence
  source: /note=“Description of Artificial Sequence: Synthetic polynucleotide”
gagatcgtgctgacccagtcccctgccaccctgtcactgtctccaggcgagagagctaccctgtcctgcaagtcctcccagtccctgctggactccggcaaccagaagaacttcctgacctggtatcagcagaagcccggccaggcccccagactgctgatctactgggcctccacccgggaatctggcgtgccctctagattctccggctccggctctggcaccgagtttaccctgaccatctccagcctgcagcccgacgacttcgccacctactactgccagaacgactactcctacccctacaccttcggccagggcaccaaggtggaaatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

SEQ ID NO: 229 - DNA, 351 bp, Artificial Sequence
  source: /note=“Description of Artificial Sequence: Synthetic polynucleotide”
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagc

SEQ ID NO: 230 - DNA, 1329 bp, Artificial Sequence
  source: /note=“Description of Artificial Sequence: Synthetic polynucleotide”
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 231 - DNA, 339 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaag

SEQ ID NO: 232 - DNA, 660 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 233 - DNA, 339 bp, Artificial Sequence
  source: /note=“Description of Artificial Sequence: Synthetic polynucleotide”
gagatcgtgctgacccagtcccccgacttccagtccgtgacccccaaagaaaaagtgaccatcacatgcaagtcctcccagtccctgctggactccggcaaccagaagaacttcctgacctggtatcagcagaagcccggccaggcccccagactgctgatctactgggcctccacccgggaatctggcgtgccctctagattctccggctccggctctggcaccgactttaccttcaccatctccagcctggaagccgaggacgccgccacctactactgccagaacgactactcctacccctacaccttcggccagggcaccaaggtggaaatcaag

SEQ ID NO: 234 - DNA, 660 bp, Artificial Sequence
  source: /note=“Description of Artificial Sequence: Synthetic polynucleotide”
gagatcgtgctgacccagtcccccgacttccagtccgtgacccccaaagaaaaagtgaccatcacatgcaagtcctcccagtccctgctggactccggcaaccagaagaacttcctgacctggtatcagcagaagcccggccaggcccccagactgctgatctactgggcctccacccgggaatctggcgtgccctctagattctccggctccggctctggcaccgactttaccttcaccatctccagcctggaagccgaggacgccgccacctactactgccagaacgactactcctacccctacaccttcggccagggcaccaaggtggaaatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

SEQ ID NO: 235 - DNA, 351 bp, Artificial Sequence
  source: /note=“Description of Artificial Sequence: Synthetic polynucleotide”
gaagtgcagctggtgcagtctggcgccgaagtgaagaagcctggcgagtccctgcggatctcctgcaagggctctggctacaccttcaccacctactggatgcactggatccggcagtccccctctaggggcctggaatggctgggcaacatctaccctggcaccggcggctccaacttcgacgagaagttcaagaacaggttcaccatctcccgggacaactccaagaacaccctgtacctgcagatgaactccctgcgggccgaggacaccgccgtgtactactgtaccagatggaccaccggaaccggcgcctattggggccagggcacaacagtgaccgtgtcctcc

SEQ ID NO: 236 - protein, 443 aa, Artificial Sequence
  source: /note=“Description of Artificial Sequence: Synthetic polypeptide”
EVQLVQSGAEVKKPGESLRISCKGSGYTFTTYWMHWIRQSPSRGLEWLGNIYPGTGGSNFDEKFKNRFTISRDNSKNTLYLQMNSLRAEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 237 - DNA, 1329 bp, Artificial Sequence
  source: /note=“Description of Artificial Sequence: Synthetic polynucleotide”
gaagtgcagctggtgcagtctggcgccgaagtgaagaagcctggcgagtccctgcggatctcctgcaagggctctggctacaccttcaccacctactggatgcactggatccggcagtccccctctaggggcctggaatggctgggcaacatctaccctggcaccggcggctccaacttcgacgagaagttcaagaacaggttcaccatctcccgggacaactccaagaacaccctgtacctgcagatgaactccctgcgggccgaggacaccgccgtgtactactgtaccagatggaccaccggaaccggcgcctattggggccagggcacaacagtgaccgtgtcctccgcttctaccaaggggcccagcgtgttccccctggccccctgctccagaagcaccagcgagagcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcaccaagacctacacctgtaacgtggaccacaagcccagcaacaccaaggtggacaagagggtggagagcaagtacggcccaccctgccccccctgcccagcccccgagttcctgggcggacccagcgtgttcctgttcccccccaagcccaaggacaccctgatgatcagcagaacccccgaggtgacctgtgtggtggtggacgtgtcccaggaggaccccgaggtccagttcaactggtacgtggacggcgtggaggtgcacaacgccaagaccaagcccagagaggagcagtttaacagcacctaccgggtggtgtccgtgctgaccgtgctgcaccaggactggctgaacggcaaagagtacaagtgtaaggtctccaacaagggcctgccaagcagcatcgaaaagaccatcagcaaggccaagggccagcctagagagccccaggtctacaccctgccacccagccaagaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaggctgaccgtggacaagtccagatggcaggagggcaacgtctttagctgctccgtgatgcacgaggccctgcacaaccactacacccagaagagcctgagcctgtccctgggc

SEQ ID NO: 238 - DNA, 339 bp, Artificial Sequence
  source: /note=“Description of Artificial Sequence: Synthetic polynucleotide”
gagatcgtgctgacccagtcccctgccaccctgtcactgtctccaggcgagagagctaccctgtcctgcaagtcctcccagtccctgctggactccggcaaccagaagaacttcctgacctggtatcagcagaagcccggccaggcccccagactgctgatctactgggcctccacccgggaatctggcgtgccctctagattctccggctccggctctggcaccgactttaccttcaccatctccagcctggaagccgaggacgccgccacctactactgccagaacgactactcctacccctacaccttcggccagggcaccaaggtggaaatcaag

SEQ ID NO: 239 - DNA, 660 bp, Artificial Sequence
  source: /note=“Description of Artificial Sequence: Synthetic polynucleotide”
gagatcgtgctgacccagtcccctgccaccctgtcactgtctccaggcgagagagctaccctgtcctgcaagtcctcccagtccctgctggactccggcaaccagaagaacttcctgacctggtatcagcagaagcccggccaggcccccagactgctgatctactgggcctccacccgggaatctggcgtgccctctagattctccggctccggctctggcaccgactttaccttcaccatctccagcctggaagccgaggacgccgccacctactactgccagaacgactactcctacccctacaccttcggccagggcaccaaggtggaaatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

SEQ ID NO: 240 - DNA, 339 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaag

SEQ ID NO: 241 - DNA, 660 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 242 - DNA, 15 bp, Artificial Sequence
  source: /note=“Description of Artificial Sequence: Synthetic oligonucleotide”
acttactggatgcac

SEQ ID NO: 243 - DNA, 51 bp, Artificial Sequence
  source: /note=“Description of Artificial Sequence: Synthetic oligonucleotide”
aatatttatcctggtactggtggttctaacttcgatgagaagttcaagaac

SEQ ID NO: 244 - DNA, 24 bp, Artificial Sequence
  source: /note=“Description of Artificial Sequence: Synthetic oligonucleotide”
tggactactgggacgggagcttat

SEQ ID NO: 245 - DNA, 21 bp, Artificial Sequence
  source: /note=“Description of Artificial Sequence: Synthetic oligonucleotide”
ggctacacattcaccacttac

SEQ ID NO: 246 - DNA, 18 bp, Artificial Sequence
  source: /note=“Description of Artificial Sequence: Synthetic oligonucleotide”
tatcctggtactggtggt

SEQ ID NO: 247 - DNA, 51 bp, Artificial Sequence
  source: /note=“Description of Artificial Sequence: Synthetic oligonucleotide”
aagtccagtcagagtctgttagacagtggaaatcaaaagaacttcttgacc

SEQ ID NO: 248 - DNA, 21 bp, Artificial Sequence
  source: /note=“Description of Artificial Sequence: Synthetic oligonucleotide”
tgggcatccactagggaatct

SEQ ID NO: 249 - DNA, 27 bp, Artificial Sequence
  source: /note=“Description of Artificial Sequence: Synthetic oligonucleotide”
cagaatgattatagttatccgtgcacg

SEQ ID NO: 250 - DNA, 39 bp, Artificial Sequence
  source: /note=“Description of Artificial Sequence: Synthetic oligonucleotide”
agtcagagtctgttagacagtggaaatcaaaagaacttc

SEQ ID NO: 251 - DNA, 9 bp, Artificial Sequence
  source: /note=“Description of Artificial Sequence: Synthetic oligonucleotide”
tgggcatcc

SEQ ID NO: 252 - DNA, 18 bp, Artificial Sequence
  source: /note=“Description of Artificial Sequence: Synthetic oligonucleotide”
gattatagttatccgtgc

SEQ ID NO: 253 - DNA, 27 bp, Artificial Sequence
  source: /note=“Description of Artificial Sequence: Synthetic oligonucleotide”
cagaatgattatagttatccgtacacg

SEQ ID NO: 254 - DNA, 18 bp, Artificial Sequence
  source: /note=“Description of Artificial Sequence: Synthetic oligonucleotide”
gattatagttatccgtac

SEQ ID NO: 255 - DNA, 24 bp, Artificial Sequence
  source: /note=“Description of Artificial Sequence: Synthetic oligonucleotide”
tggactactgggacgggagcttac

SEQ ID NO: 256 - DNA, 15 bp, Artificial Sequence
  source: /note=“Description of Artificial Sequence: Synthetic oligonucleotide”
acctactggatgcac

SEQ ID NO: 257 - DNA, 51 bp, Artificial Sequence
  source: /note=“Description of Artificial Sequence: Synthetic oligonucleotide”
aacatctatcctggcaccggcggctccaacttcgacgagaagttcaagaac

SEQ ID NO: 258 - DNA, 24 bp, Artificial Sequence
  source: /note=“Description of Artificial Sequence: Synthetic oligonucleotide”
tggacaaccggcacaggcgcttat

SEQ ID NO: 259 - DNA, 21 bp, Artificial Sequence
  source: /note=“Description of Artificial Sequence: Synthetic oligonucleotide”
ggctacaccttcaccacctac

SEQ ID NO: 260 - DNA, 18 bp, Artificial Sequence
  source: /note=“Description of Artificial Sequence: Synthetic oligonucleotide”
tatcctggcaccggcggc

SEQ ID NO: 261 - DNA, 51 bp, Artificial Sequence
  source: /note=“Description of Artificial Sequence: Synthetic oligonucleotide”
aagtcctcccagtccctgctggactccggcaaccagaagaacttcctgacc

SEQ ID NO: 262 - DNA, 21 bp, Artificial Sequence
  source: /note=“Description of Artificial Sequence: Synthetic oligonucleotide”
tgggcctccacccgggaatct

SEQ ID NO: 263 - DNA, 27 bp, Artificial Sequence
  source: /note=“Description of Artificial Sequence: Synthetic oligonucleotide”
cagaacgactactcctacccctacacc

SEQ ID NO: 264 - DNA, 39 bp, Artificial Sequence
  source: /note=“Description of Artificial Sequence: Synthetic oligonucleotide”
tcccagtccctgctggactccggcaaccagaagaacttc

SEQ ID NO: 265 - DNA, 9 bp, Artificial Sequence
  source: /note=“Description of Artificial Sequence: Synthetic oligonucleotide”
tgggcctcc

SEQ ID NO: 266 - DNA, 18 bp, Artificial Sequence
  source: /note=“Description of Artificial Sequence: Synthetic oligonucleotide”
gactactcctacccctac

SEQ ID NO: 267 - DNA, 15 bp, Artificial Sequence
  source: /note=“Description of Artificial Sequence: Synthetic oligonucleotide”
acctactggatgcac

SEQ ID NO: 268 - DNA, 51 bp, Artificial Sequence
  source: /note=“Description of Artificial Sequence: Synthetic oligonucleotide”
aatatctaccccggcaccggcggctctaacttcgacgagaagtttaagaat

SEQ ID NO: 269 - DNA, 24 bp, Artificial Sequence
  source: /note=“Description of Artificial Sequence: Synthetic oligonucleotide”
tggactaccggcacaggcgcctac

SEQ ID NO: 270 - DNA, 21 bp, Artificial Sequence
  source: /note=“Description of Artificial Sequence: Synthetic oligonucleotide”
ggctacaccttcactacctac

SEQ ID NO: 271 - DNA, 18 bp, Artificial Sequence
  source: /note=“Description of Artificial Sequence: Synthetic oligonucleotide”
taccccggcaccggcggc

SEQ ID NO: 272 - DNA, 51 bp, Artificial Sequence
  source: /note=“Description of Artificial Sequence: Synthetic oligonucleotide”
aaatctagtcagtcactgctggatagcggtaatcagaagaacttcctgacc

SEQ ID NO: 273 - DNA, 21 bp, Artificial Sequence
  source: /note=“Description of Artificial Sequence: Synthetic oligonucleotide”
tgggcctctactagagaatca

SEQ ID NO: 274 - DNA, 27 bp, Artificial Sequence
  source: /note=“Description of Artificial Sequence: Synthetic oligonucleotide”
cagaacgactatagctacccctacacc

SEQ ID NO: 275 - DNA, 39 bp, Artificial Sequence
  source: /note=“Description of Artificial Sequence: Synthetic oligonucleotide”
agtcagtcactgctggatagcggtaatcagaagaacttc

SEQ ID NO: 276 - DNA, 9 bp, Artificial Sequence
  source: /note=“Description of Artificial Sequence: Synthetic oligonucleotide”
tgggcctct

SEQ ID NO: 277 - DNA, 18 bp, Artificial Sequence
  source: /note=“Description of Artificial Sequence: Synthetic oligonucleotide”
gactatagctacccctac

SEQ ID NO: 278 - DNA, 51 bp, Artificial Sequence
  source: /note=“Description of Artificial Sequence: Synthetic oligonucleotide”
aacatctaccctggcaccggcggctccaacttcgacgagaagttcaagaac

SEQ ID NO: 279 - DNA, 24 bp, Artificial Sequence
  source: /note=“Description of Artificial Sequence: Synthetic oligonucleotide”
tggaccaccggaaccggcgcctat

SEQ ID NO: 280 - DNA, 18 bp, Artificial Sequence
  source: /note=“Description of Artificial Sequence: Synthetic oligonucleotide”
taccctggcaccggcggc

SEQ ID NO: 281 - protein, 440 aa, Artificial Sequence
  source: /note=“Description of Artificial Sequence: Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 282 - protein, 214 aa, Artificial Sequence
  source: /note=“Description of Artificial Sequence: Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 283 - protein, 447 aa, Artificial Sequence
  source: /note=“Description of Artificial Sequence: Synthetic polypeptide”
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 284 - protein, 218 aa, Artificial Sequence
  source: /note=“Description of Artificial Sequence: Synthetic polypeptide”
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 285 - DNA, 51 bp, Artificial Sequence
  source: /note=“Description of Artificial Sequence: Synthetic oligonucleotide”
aagtccagtcagagtctgttagacagtggaaatcaaaagaacttcttaacc

SEQ ID NO: 286 - protein, 10 aa, Artificial Sequence
  source: /note=“Description of Artificial Sequence: Synthetic peptide”
GYTFTTYWMH

SEQ ID NO: 287-499 - skipped (213 entries)

SEQ ID NO: 500 - protein, 126 aa, Artificial Sequence
  source: /note=“Description of Artificial Sequence: Synthetic polypeptide”
QVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKVGYDSSGYYRDYYGMDVWGQGTTVTVSS

SEQ ID NO: 501 - protein, 110 aa, Artificial Sequence
  source: /note=“Description of Artificial Sequence: Synthetic polypeptide”
EIVLTQSPGTLSLSPGERATLSCRASQSVSSSYLAWYQQKPGQAPRLLIYGTSSRATGISDRFSGSGSGTDFTLTISRLEPEDFAVYYCQHYGNSPPKFTFGPGTKLEIK

SEQ ID NO: 502 - protein, 495 aa, Artificial Sequence
  source: /note=“Description of Artificial Sequence: Synthetic polypeptide”
MALPVTALLLPLALLLHAARPQVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKVGYDSSGYYRDYYGMDVWGQGTTVTVSSGGGGSGGGGSGGGGSEIVLTQSPGTLSLSPGERATLSCRASQSVSSSYLAWYQQKPGQAPRLLIYGTSSRATGISDRFSGSGSGTDFTLTISRLEPEDFAVYYCQHYGNSPPKFTFGPGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 503 - DNA, 1485 bp, Artificial Sequence
  source: /note=“Description of Artificial Sequence: Synthetic polynucleotide”
atggccctccctgtcaccgccctgctgcttccgctggctcttctgctccacgccgctcggccccaagtgcagctcgtggagtcaggcggaggactggtgcagcccgggggctccctgagactttcctgcgcggcatcgggttttaccttctcctcctatgctatgtcctgggtgcgccaggccccgggaaagggactggaatgggtgtccgcaatcagcggtagcgggggctcaacatactacgccgactccgtcaagggtcgcttcactatttcccgggacaactccaagaataccctgtacctccaaatgaacagcctcagggccgaggatactgccgtgtactactgcgccaaagtcggatacgatagctccggttactaccgggactactacggaatggacgtgtggggacagggcaccaccgtgaccgtgtcaagcggcggaggcggttcaggagggggaggctccggcggtggagggtccgaaatcgtcctgactcagtcgcctggcactctgtcgttgtccccgggggagcgcgctaccctgtcgtgtcgggcgtcgcagtccgtgtcgagctcctacctcgcgtggtaccagcagaagcccggacaggcccctagacttctgatctacggcacttcttcacgcgccaccgggatcagcgacaggttcagcggctccggctccgggaccgacttcaccctgaccattagccggctggagcctgaagatttcgccgtgtattactgccaacactacggaaactcgccgccaaagttcacgttcggacccggaaccaagctggaaatcaagaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 504 - protein, 246 aa, Artificial Sequence
  source: /note=“Description of Artificial Sequence: Synthetic polypeptide”
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKMGWSSGYLGAFDIWGQGTTVTVSSGGGGSGGGGSGGGGSEIVLTQSPGTLSLSPGERATLSCRASQSVASSFLAWYQQKPGQAPRLLIYGASGRATGIPDRFSGSGSGTDFTLTISRLEPEDFAVYYCQHYGGSPRLTFGGGTKVDIK

SEQ ID NO: 505 - DNA, 739 bp, Artificial Sequence
  source: /note=“Description of Artificial Sequence: Synthetic polynucleotide”
gaagtccaactggtggagtccgggggagggctcgtgcagcccggaggcagccttcggctgtcgtgcgccgcctccgggttcacgttctcatcctacgcgatgtcgtgggtcagacaggcaccaggaaagggactggaatgggtgtccgccattagcggctccggcggtagcacctactatgccgactcagtgaagggaaggttcactatctcccgcgacaacagcaagaacaccctgtacctccaaatgaactctctgcgggccgaggataccgcggtgtactattgcgccaagatgggttggtccagcggatacttgggagccttcgacatttggggacagggcactactgtgaccgtgtcctccgggggtggcggatcgggaggcggcggctcgggtggagggggttccgaaatcgtgttgacccagtcaccgggaaccctctcgctgtccccgggagaacgggctacactgtcatgtagagcgtcccagtccgtggcttcctcgttcctggcctggtaccagcagaagccgggacaggcaccccgcctgctcatctacggagccagcggccgggcgaccggcatccctgaccgcttctccggttccggctcgggcaccgactttactctgaccattagcaggcttgagcccgaggattttgccgtgtactactgccaacactacggggggagccctcgcctgaccttcggaggcggaactaaggtcgatatcaaaa

SEQ ID NO: 506 - protein, 122 aa, Artificial Sequence
  source: /note=“Description of Artificial Sequence: Synthetic polypeptide”
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKMGWSSGYLGAFDIWGQGTTVTVSS

SEQ ID NO: 507 - protein, 109 aa, Artificial Sequence
  source: /note=“Description of Artificial Sequence: Synthetic polypeptide”
EIVLTQSPGTLSLSPGERATLSCRASQSVASSFLAWYQQKPGQAPRLLIYGASGRATGIPDRFSGSGSGTDFTLTISRLEPEDFAVYYCQHYGGSPRLTFGGGTKVDIK

SEQ ID NO: 508 - protein, 490 aa, Artificial Sequence
  source: /note=“Description of Artificial Sequence: Synthetic polypeptide”
MALPVTALLLPLALLLHAARPEVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKMGWSSGYLGAFDIWGQGTTVTVSSGGGGSGGGGSGGGGSEIVLTQSPGTLSLSPGERATLSCRASQSVASSFLAWYQQKPGQAPRLLIYGASGRATGIPDRFSGSGSGTDFTLTISRLEPEDFAVYYCQHYGGSPRLTFGGGTKVDIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 509 - DNA, 1470 bp, Artificial Sequence
  source: /note=“Description of Artificial Sequence: Synthetic polynucleotide”
atggccctccctgtcaccgccctgctgcttccgctggctcttctgctccacgccgctcggcccgaagtccaactggtggagtccgggggagggctcgtgcagcccggaggcagccttcggctgtcgtgcgccgcctccgggttcacgttctcatcctacgcgatgtcgtgggtcagacaggcaccaggaaagggactggaatgggtgtccgccattagcggctccggcggtagcacctactatgccgactcagtgaagggaaggttcactatctcccgcgacaacagcaagaacaccctgtacctccaaatgaactctctgcgggccgaggataccgcggtgtactattgcgccaagatgggttggtccagcggatacttgggagccttcgacatttggggacagggcactactgtgaccgtgtcctccgggggtggcggatcgggaggcggcggctcgggtggagggggttccgaaatcgtgttgacccagtcaccgggaaccctctcgctgtccccgggagaacgggctacactgtcatgtagagcgtcccagtccgtggcttcctcgttcctggcctggtaccagcagaagccgggacaggcaccccgcctgctcatctacggagccagcggccgggcgaccggcatccctgaccgcttctccggttccggctcgggcaccgactttactctgaccattagcaggcttgagcccgaggattttgccgtgtactactgccaacactacggggggagccctcgcctgaccttcggaggcggaactaaggtcgatatcaaaaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 510 - protein, 122 aa, Artificial Sequence
  source: /note=“Description of Artificial Sequence: Synthetic polypeptide”
QIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSA

SEQ ID NO: 511 - protein, 107 aa, Artificial Sequence
  source: /note=“Description of Artificial Sequence: Synthetic polypeptide”
DVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIK

SEQ ID NO: 512 - protein, 244 aa, Artificial Sequence
  source: /note=“Description of Artificial Sequence: Synthetic polypeptide”
QIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSAGGGGSGGGGSGGGGSDVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIK

SEQ ID NO: 513 - protein, 467 aa, Artificial Sequence
  source: /note=“Description of Artificial Sequence: Synthetic polypeptide”
QIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSAGGGGSGGGGSGGGGSDVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 514 - protein, 117 aa, Artificial Sequence
  source: /note=“Description of Artificial Sequence: Synthetic polypeptide”
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 515 - protein, 111 aa, Artificial Sequence
  source: /note=“Description of Artificial Sequence: Synthetic polypeptide”
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 516 - protein, 249 aa, Artificial Sequence
  source: /note=“Description of Artificial Sequence: Synthetic polypeptide”
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 517 - protein, 472 aa, Artificial Sequence
  source: /note=“Description of Artificial Sequence: Synthetic polypeptide”
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 518 - protein, 117 aa, Artificial Sequence
  source: /note=“Description of Artificial Sequence: Synthetic polypeptide”
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 519 - protein, 111 aa, Artificial Sequence
  source: /note=“Description of Artificial Sequence: Synthetic polypeptide”
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 520 - protein, 243 aa, Artificial Sequence
  source: /note=“Description of Artificial Sequence: Synthetic polypeptide”
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSSGGGGSGGGGSGGGGSDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 521 - protein, 466 aa, Artificial Sequence
  source: /note=“Description of Artificial Sequence: Synthetic polypeptide”
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSSGGGGSGGGGSGGGGSDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 522 - protein, 117 aa, Artificial Sequence
  source: /note=“Description of Artificial Sequence: Synthetic polypeptide”
QIQLVQSGPELKKPGETVKISCKASGYTFTHYSMNWVKQAPGKGLKWMGRINTETGEPLYADDFKGRFAFSLETSASTAYLVINNLKNEDTATFFCSNDYLYSCDYWGQGTTLTVSS

SEQ ID NO: 523 - protein, 111 aa, Artificial Sequence; identical to SEQ ID NO: 519
  source: /note=“Description of Artificial Sequence: Synthetic polypeptide”

SEQ ID NO: 524 - protein, 243 aa, Artificial Sequence
  source: /note=“Description of Artificial Sequence: Synthetic polypeptide”
QIQLVQSGPELKKPGETVKISCKASGYTFTHYSMNWVKQAPGKGLKWMGRINTETGEPLYADDFKGRFAFSLETSASTAYLVINNLKNEDTATFFCSNDYLYSCDYWGQGTTLTVSSGGGGSGGGGSGGGGSDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 525 - protein, 466 aa, Artificial Sequence
  source: /note=“Description of Artificial Sequence: Synthetic polypeptide”
QIQLVQSGPELKKPGETVKISCKASGYTFTHYSMNWVKQAPGKGLKWMGRINTETGEPLYADDFKGRFAFSLETSASTAYLVINNLKNEDTATFFCSNDYLYSCDYWGQGTTLTVSSGGGGSGGGGSGGGGSDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 526 - protein, 4 aa, Artificial Sequence
  source: /note=“Description of Artificial Sequence: Synthetic peptide”
RGDS

SEQ ID NO: 527 - protein, 15 aa, Artificial Sequence
  source: /note=“Description of Artificial Sequence: Synthetic peptide”
RASKGVSTSGYSYLH

SEQ ID NO: 528 - protein, 7 aa, Artificial Sequence
  source: /note=“Description of Artificial Sequence: Synthetic peptide”
LASYLES

SEQ ID NO: 529 - protein, 9 aa, Artificial Sequence
  source: /note=“Description of Artificial Sequence: Synthetic peptide”
QHSRDLPLT

SEQ ID NO: 530 - protein, 5 aa, Artificial Sequence
  source: /note=“Description of Artificial Sequence: Synthetic peptide”
NYYMY

SEQ ID NO: 531 - protein, 17 aa, Artificial Sequence
  source: /note=“Description of Artificial Sequence: Synthetic peptide”
GINPSNGGTNFNEKFKN

SEQ ID NO: 532 - protein, 11 aa, Artificial Sequence
  source: /note=“Description of Artificial Sequence: Synthetic peptide”
RDYRFDMGFDY

SEQ ID NO: 533 - protein, 8 aa, Artificial Sequence
  source: /note=“Description of Artificial Sequence: Synthetic peptide”
GYTFTTYW

SEQ ID NO: 534 - protein, 8 aa, Artificial Sequence
  source: /note=“Description of Artificial Sequence: Synthetic peptide”
IYPGTGGS

SEQ ID NO: 535 - protein, 10 aa, Artificial Sequence
  source: /note=“Description of Artificial Sequence: Synthetic peptide”
TRWTTGTGAY

SEQ ID NO: 536 - protein, 12 aa, Artificial Sequence
  source: /note=“Description of Artificial Sequence: Synthetic peptide”
QSLLDSGNQKNF

SEQ ID NO: 537 - protein, 8 aa, Artificial Sequence
  source: /note=“Description of Artificial Sequence: Synthetic peptide”
GFNIEDYY

SEQ ID NO: 538 - protein, 8 aa, Artificial Sequence
  source: /note=“Description of Artificial Sequence: Synthetic peptide”
IDPENDET

SEQ ID NO: 539 - protein, 7 aa, Artificial Sequence
  source: /note=“Description of Artificial Sequence: Synthetic peptide”
AFRGGVY

SEQ ID NO: 540 - protein, 11 aa, Artificial Sequence
  source: /note=“Description of Artificial Sequence: Synthetic peptide”
QSLLDSDGKTY

SEQ ID NO: 541 - protein, 6 aa, Artificial Sequence
  source: /note=“Description of Artificial Sequence: Synthetic peptide”
QGIRNN

SEQ ID NO: 542 - protein, 3 aa, Artificial Sequence
  source: /note=“Description of Artificial Sequence: Synthetic peptide”
AAS

SEQ ID NO: 543 - protein, 9 aa, Artificial Sequence
  source: /note=“Description of Artificial Sequence: Synthetic peptide”
LQHHSYPLT

SEQ ID NO: 544 - protein, 107 aa, Artificial Sequence
  source: /note=“Description of Artificial Sequence: Synthetic polypeptide”
DIQMTQSPSSLSASVGDRVTITCRASQGIRNNLAWYQQKPGKAPKRLIYAASNLQSGVPSRFTGSGSGTEFTLIVSSLQPEDFATYYCLQHHSYPLTSGGGTKVEIK

SEQ ID NO: 545 - protein, 8 aa, Artificial Sequence
  source: /note=“Description of Artificial Sequence: Synthetic peptide”
GFTFSSYA

SEQ ID NO: 546 - protein, 8 aa, Artificial Sequence
  source: /note=“Description of Artificial Sequence: Synthetic peptide”
ISGSGGST

SEQ ID NO: 547 - protein, 9 aa, Artificial Sequence
  source: /note=“Description of Artificial Sequence: Synthetic peptide”
AGSSGWSEY

SEQ ID NO: 548 - protein, 116 aa, Artificial Sequence
  source: /note=“Description of Artificial Sequence: Synthetic polypeptide”
EVQVLESGGGLVQPGGSLRLSCAASGFTFSSYAMSWVRQAPGKGLEWVSAISGSGGSTNYADSVKGRFTISRDNSKNTLYLQMNSLRAEDTAVYYCAGSSGWSEYWGQGTLVTVSS

(19) United States
(12) Patent Application Publication
(10) Pub. No.: US 2022/0088075 A1
(43) Pub. Date: Mar. 24, 2022

(54) COMBINATION THERAPIES OF EGFRVIII CHIMERIC ANTIGEN RECEPTORS AND PD-1 INHIBITORS

(71) Applicants: The Trustees of the University of Pennsylvania, Philadelphia, PA (US); Novartis AG, Basel (CH)

(72) Inventors: Donald M. O'Rourke, Wynnewood, PA (US); Stephen Joseph Bagley, Blue Bell, PA (US); Randi Isaacs, Basking Ridge, NJ (US); Arati Suvas Desai, Philadelphia, PA (US)

(21) Appl. No.: 17/432,868

(22) PCT Filed: Feb. 21, 2020
(86) PCT No.: PCT/US2020/019264

§ 371 Date: Aug. 20, 2021

Related U.S. Application Data
(60) Provisional application No. 62/809,245, filed on Feb. 22, 2019.

(51) Int. Cl. A61K 35/17; C07K 16/28

(52) U.S. Cl. CPC A61K 35/17; C07K 16/2863; C07K 16/2818; C07K 2319/33; C07K 2317/76; C07K 2319/03; C07K 2317/622

(57) ABSTRACT

Provided are compositions and methods for treating diseases, e.g., cancers, e.g., diseases associated with expression of an antigen, e.g., EGFRvIII, comprising administering a cell that expresses a chimeric antigen receptor (CAR) specific to the antigen, e.g., EGFRvIII, in combination with a PD-1 inhibitor.

### CROSS REFERENCE TO RELATED APPLICATIONS

This application claims priority to U.S. Application Ser. No. 62/809,245, filed Feb. 22, 2019, the contents of which are incorporated herein by reference in their entireties.

### SEQUENCE LISTING

The instant application contains a Sequence Listing which has been submitted electronically in ASCII format and is hereby incorporated by reference in its entirety. Said ASCII copy, created on Feb. 20, 2020, is named N2067-7158WO_SL.txt and is 476,717 bytes in size.

### FIELD OF THE INVENTION

The present invention relates generally to the use of cells, e.g., immune effector cells, engineered to express a Chimeric Antigen Receptor (CAR) that targets an antigen, e.g., EGFRvIII, in combination with PD-1 inhibitors to treat a disease.

### BACKGROUND OF THE INVENTION

Although the central nervous system (CNS) is often considered to be immunologically privileged (Okada et al., 2009, Crit Rev Immunol 29:1-42), recent vaccine studies in patients with malignant glioma demonstrated positive results (Aguilar et al., 2012, Curr Treat Options Oncol 13:437-450; Ruzevick, et al., 2012, Neurosurg Clin N Am 23:459-470; 15; and Okada et al., 2011, J Clin Oncol 29:330-336). However, vaccine efficacy, which relies on intact host-immune activity, can suffer from systemic suppression of immunity due to tumor expression of immunosuppressive cytokines as well as chemo- and radiotherapy. On the other hand, adoptive cell transfer (ACT) therapy with autologous T-cells, especially with T-cells transduced with Chimeric Antigen Receptors (CARs), has shown promise in pilot hematologic cancer trials (Kalos et al., 2011, Sci Transl Med 3(95):95ra73; and Porter et al., 2011, New England Journal of Medicine 365:725-733).

Enhanced expression of epidermal growth factor receptor (EGFR) is frequently detected in a variety of carcinomas, including breast, lung, head and neck, as well as glioblastoma. Spontaneous rearrangements within the EGF receptor gene were first identified in primary human glioblastoma tumors, and in nearly all cases the alterations have been reported in tumors with EGFR amplification. Three different types of mutants result from these rearrangements. The most common of these is the Type III EGF deletion-mutant receptor (EGFRvIII), which is characterized by the deletion of exons 2-7 in the EGFR mRNA. These deletions correspond to cDNA nucleotides 275-1075, which encode amino acids 6-276, presumably through alternative splicing or rearrangements. Deletion of 801 bp within the extracellular domain of the EGFR gene causes an in-frame truncation of the normal EGFR protein, resulting in a 145-kDa receptor, thereby creating a tumor specific and immunogenic epitope (reviewed in Hatanpaa et al., 2010, Neoplasia 12:675-684; Mukasa et al., 2010, Proc Natl Acad Sci USA 107:2616-2621). EGFRvIII expression has been seen in many tumor types, including glioblastoma multiforme (GBM), but is rarely observed in normal tissue. EGFRvIII is expressed in 24% to 67% of GBM cases, and in patients surviving ≥1 year, the expression of EGFRvIII is an independent negative prognostic indicator (Heimberger et al., 2005, Clin. Cancer Res. 11:1462-1466; Heimberger et al., 2005, J Transl. Med 3:38).

GBM is the most common and most malignant of glial tumors, which in turn are the most common type of primary brain tumor (Ostrom et al., 2016, Neuro. Oncol. 18:v1-v75). GBMs are divided into two groups: Isocitrate Dehydrogenase (IDH)—wildtype (about 90% of cases, known as “primary” or “de novo” GBM) and IDH-mutant (about 10% of cases, known as “secondary” GBM due to the evolution of these tumors from lower grade precursor gliomas) (Louis et al., 2016, [ ]). IDH mutation status is significantly associated with clinical outcomes, with IDH-mutant tumors being independently associated with improved prognosis. The other major molecular marker of clinical significance in GBM is the methylation status of the promoter of the 06-methulguaninne-DNA-methyltransferase (MGMT) gene. MGMT is a DNA damage repair protein that removes the guanine-alkyl group and prevents apoptosis (Ludwig and Kornblum, 2017, J. Neuroncol. 134:505-512), thus, mediating resistance to alkylating chemotherapy. Loss of MGMT makes tumors more sensitive to alkylating agents. Expression of MGMT is tightly regulated by methylation of its promoter, which leads to decreased expression of this protein and ultimately increased response to alkylating chemotherapy (Ludwig and Kornblum, 2017, J. Neuroncol. 134:505-512). MGMT promoter methylation is associated with improved prodgnosis even without regard to whether alkylating chemotherapy is administered (Ludwig and Kornblum, 2017, J. Neuroncol. 134:505-512).

The treatment standard for GBM, since 2005, has been maximal safe surgical resection followed by adjuvant radiation therapy (RT) with oral alkylating agent temozolomide (TMZ) administered once daily concurrently (Stupp et al., 2005, NJEM 352:9987-996). Their remains a need in the art for treatments that provide improved overall survival if the tumor recurs after the standard treatment. Further, in patients whose tumors harbor an unmethylated MGMT promoter, see very little benefit with TMZ treatment (Hegi et al., 2005, NJEM 352:997-1003; Hegi and Stupp, 2015, Neuro. Oncol. 17:1425-1427). Thus, effective treatment for MGMT-unmethylated GBM represents a significant unmet medical need in the art.

### SUMMARY OF THE INVENTION

The present disclosure features, at least in part, methods and compositions for treating a disease (e.g., cancer), e.g., disease associated with an antigen, e.g., disease associated with the expression of EGFRvIII, e.g., a cancer, in a subject by using a combination therapy that includes a cell, e.g., an immune effector cell, expressing a chimeric antigen receptor (CAR) that specifically binds to an antigen, e.g., antigen described herein, e.g., EGFRvIII (also referred to herein as a “EGFRvIII CAR-expressing cell”) (also referred to herein as a “CAR therapy”) and an inhibitor of Programmed Death-1 (also referred to herein as a “PD-1 inhibitor”). In some embodiments, the CAR that specifically binds to the antigen, e.g., EGFRvIII, includes an antigen binding domain, e.g., an EGFRvIII binding domain, a transmembrane domain, and an intracellular signaling domain, e.g., as described herein. In some embodiments, the PD-1 inhibitor is an antibody molecule, a polypeptide, a small molecule, or a polynucleotide, e.g., an inhibitory nucleic acid. In one embodiment, the PD-1 inhibitor is an antibody molecule, e.g., an antibody molecule described herein. Without wishing to be bound by theory, treating a subject having a disease (e.g., cancer), e.g., disease associated with EGFRvIII expression, e.g., a cancer described herein, with a combination therapy that includes a CAR-expressing cell (e.g., EGFRvIII CAR-expressing cell) and a PD-1 inhibitor is believed to result in improved inhibition or reduction of tumor progression in the subject, e.g., as compared to treating a subject having the disease with either a CAR-expressing cell (e.g., EGFRvIII CAR-expressing cell) or a PD-1 inhibitor alone. For example, inhibition of the PD-1/PD-L1 interaction, in combination with the CAR therapy, can result in one or more of: (i) activation (or reactivation) of CAR-expressing cells (e.g., EGFRvIII CAR-expressing cells); (ii) expansion in a population of CAR-expressing cells; (iii) sustained duration of a therapeutic response to a CAR therapy; (iv) increased persistence of the CAR therapy, (v) reduction of exhausted effector T cells function, (vi) reversal or relief of T cell exhaustion, (vii) increased cytokine (e.g., IL-6, or IL-2) levels; or (viii) decreased expression of checkpoint inhibitors (e.g., one or more of PD-1, TIM-3 or LAG-3) on immune effector cells (e.g., CD4+ and/or CD8+ cells, e.g., CAR-expressing immune effector cells), thus resulting in an improved therapeutic outcome in a subject treated with the combination therapy, e.g., compared to a subject receiving a CAR-therapy alone or a PD-1 inhibitor alone.

Accordingly, in one aspect, the disclosure features a method of treating a subject having a disease (e.g., cancer), e.g., a disease associated with an antigen, e.g., a disease associated with expression of EGFRvIII, e.g., a cancer as described herein. The method includes administering to the subject a cell, e.g., a population of cells, comprising, e.g., expressing a CAR that specifically binds to an antigen, e.g., EGFRvIII (also referred to herein as a CAR therapy), and a PD-1 inhibitor. In one embodiment, the CAR-expressing cell and the PD-1 inhibitor is administered sequentially. In one embodiment, the PD-1 inhibitor is administered prior to administration of the CAR-expressing cell (e.g., EGFRvIII CAR-expressing cell). In one embodiment, the PD-1 inhibitor is administered after the administration of the CAR-expressing cell (e.g., EGFRvIII CAR-expressing cell). In one embodiment, the PD-1 inhibitor and CAR-expressing cell (e.g., EGFRvIII CAR-expressing cell) are administered simultaneously or concurrently.

In embodiments, the CAR-expressing cell e.g., EGFRvIII CAR-expressing cell described herein, and the PD-1 inhibitor are administered sequentially, e.g., in any order. In one embodiment, the combination is administered in a treatment interval. In one embodiment, the treatment interval comprises a single dose of the PD-1 inhibitor and a single dose of the CAR-expressing cell (e.g., in any order). In another embodiment, the treatment interval comprises multiple doses (e.g., a first and second dose) of the PD-1 inhibitor and a dose of the CAR-expressing cell (e.g., in any order).

In a related aspect, the disclosure provides a method of treating a subject having a cancer. The method comprises administering to the subject:

(i) a CAR therapy comprising a population of immune effector cells, comprising, e.g., expressing, a CAR, wherein the CAR comprises an antigen (e.g., an EGFRvIII) binding domain, a transmembrane domain, and an intracellular signaling domain; and

(ii) a PD-1 inhibitor.

In some embodiments, the dose of the PD-1 inhibitor, e.g., anti-PD-1 antibody molecule, is about 200 mg to about 450 mg, e.g., about 300 mg to about 400 mg, e.g., administered every 2 weeks, 3 weeks, 4 weeks, or 5 weeks.

In another aspect, the disclosure provides a method of treating a subject having a cancer.

The method comprises administering to the subject:

(i) a CAR therapy comprising a population of immune effector cells comprising, e.g., expressing, a CAR, wherein the CAR comprises an antigen (e.g., an EGFRvIII) binding domain, a transmembrane domain, and an intracellular signaling domain; and

(ii) a PD-1 inhibitor.

In some embodiments, administration of the PD-1 inhibitor is initiated 20 days or less after administration of the CAR therapy. For example, administration of the PD-1 inhibitor is initiated 16 days or less, 15 days or less, 14 days or less, 13 days or less, 12 days or less, 11 days or less, 10 days or less, 9 days or less, 8 days or less, 7 days or less, 6 days or less, 5 days or less, 4 days or less, 3 days or less, 2 days or less, 1 day or less before or after administration of the CAR therapy; or on the same day as administration of the CAR therapy.

In another aspect, the disclosure provides a method of treating a subject having a cancer. The method comprises administering to the subject:

(i) a CAR therapy comprising a population of immune effector cells comprising, e.g., expressing, a CAR, wherein the CAR comprises an antigen (e.g., an EGFRvIII) binding domain, a transmembrane domain, and an intracellular signaling domain; and

(ii) a PD-1 inhibitor.

In some embodiments, administration of the PD-1 inhibitor is initiated after the subject has, or is identified as having, one or more of the following:
(a) a partial or no detectable response to the CAR therapy,
(b) a relapsed cancer after the CAR therapy,
(c) a cancer refractory to the CAR therapy; or
(d) a progressive form of the cancer after the CAR therapy.

In yet another aspect, the disclosure provides a method of treating a subject having a cancer. The method comprises administering to the subject:

(i) a CAR therapy comprising a population of immune effector cells comprising, e.g., expressing, a chimeric antigen receptor (CAR), wherein the CAR comprises an antigen (e.g., an EGFRvIII) binding domain, a transmembrane domain, and an intracellular signaling domain; and (ii) a PD-1 inhibitor.

In some embodiments, administration of the PD-1 inhibitor is initiated after administration of the CAR therapy, and the subject does not have, or has not been identified as having, one or more of the following:

(a) a partial or no detectable response to the CAR therapy,

(b) a relapsed cancer after the CAR therapy,

(c) a cancer refractory to the CAR therapy; or

(d) a progressive form of the cancer.

In another aspect, the disclosure provides a CAR therapy for use in combination with a PD-1 inhibitor in any of the methods disclosed herein. In other embodiments, disclosed herein is the use of a CAR therapy in combination with a PD-1 inhibitor in the preparation of a medicament for treating a disorder, e.g., a proliferative disorder, e.g., a cancer.

Additional features or embodiments of any of the methods, uses, compositions or combinations disclosed herein include one or more of the following:

In some embodiments, one or more, e.g., 1, 2, 3, 4, or 5 or more, subsequent doses of the PD-1 inhibitor can be administered. In one embodiment, up to 6 doses of the PD-1 inhibitor are administered.

In some embodiments, the method or use further comprises evaluating the presence or absence of CRS in the subject. In one embodiment, the subject does not have, or is identified, as not having CRS, e.g., severe CRS (e.g., CRS grade 3 or grade 4), after the CAR therapy.

In other embodiments, administration of the PD-1 inhibitor is initiated after the subject is identified as not having CRS, e.g., severe CRS (e.g., CRS grade 3 or grade 4), after the CAR therapy.

In other embodiments, administration of the PD-1 inhibitor is initiated after treatment of CRS, e.g., CRS resolution, after the CAR therapy. In one embodiment, the CRS is resolved to grade 1. In an embodiment, the CRS is resolved to undetectable levels.

Where the treatment interval comprises a single dose of the PD-1 inhibitor and a single dose of the CAR-expressing cell, in certain embodiments, the dose of PD-1 inhibitor and the dose of the CAR-expressing cell are administered simultaneously or concurrently. For example, the dose of the PD-1 inhibitor and the dose of the CAR-expressing cell are administered within 20 days, 18 days, 16 days, 15 days, 12 days, 10 days, 9 days, 8 days, 7 days, 6 days, 5 days, 4 days, 3 days, 2 days, 1 day, 24 hours, 12 hours, 6 hours, 4 hours, 2 hours, or less of each other. In embodiments, the treatment interval is initiated upon administration of the first-administered dose and completed upon administration of the later-administered dose.

Where the treatment interval comprises a single dose of the PD-1 inhibitor and a single dose of the CAR-expressing cell, in certain embodiments, the dose of the PD-1 inhibitor and the dose of the CAR-expressing cell are administered sequentially. In embodiments, the dose of the CAR-expressing cell is administered prior to the dose of the PD-1 inhibitor, and the treatment interval is initiated upon administration of the dose of the CAR-expressing cell and completed upon administration of the dose of the PD-1 inhibitor. In other embodiments, the dose of the PD-1 inhibitor is administered prior to the dose of the CAR-expressing cell, and the treatment interval is initiated upon administration of the dose of the PD-1 inhibitor and completed upon administration of the dose of the CAR-expressing cell. In one embodiment, the treatment interval further comprises one or more, e.g., 1, 2, 3, 4, or 5 or more, subsequent doses of the PD-1 inhibitor. In such embodiments, the treatment interval comprises two, three, four, five, six, or more, doses of PD-1 inhibitor and one dose of the CAR-expressing cell. In one embodiment, the dose of the CAR-expressing cell is administered at least 2 days, at least 3 days, at least 4 days, at least 5 days, at least 6 days, at least 7 days, at least 8 days, at least 9 days, at least 10 days, at least 8 days, at least 9 days, at least 10 days, at least 11 days, at least 12 days, at least 13 days, or at least 2 weeks before or after a dose of PD-1 inhibitor is administered. In embodiments where more than one dose of PD-1 inhibitor is administered, the dose of the CAR-expressing cell is administered at least 2 days, at least 3 days, at least 4 days, at least 5 days, at least 6 days, at least 7 days, at least 8 days, at least 9 days, at least 10 days, at least 8 days, at least 9 days, at least 10 days, at least 11 days, at least 12 days, at least 13 days, or at least 2 weeks before or after the first dose of PD-1 inhibitor is administered or after the initiation of the treatment interval. In one embodiment, the dose of the PD-1 inhibitor is administered about 25-40 days (e.g., about 25-30, 30-35, or 35-40 days, e.g., about 35 days) or about 2-7 weeks (e.g., 2, 3, 4, 5, 6, or 7 weeks) after the dose of the CAR-expressing cell is administered. In embodiments, where more than one dose of PD-1 inhibitor is administered, the second PD-1 inhibitor dose is administered about 15-30 days (e.g., about 15-20, 20-25, or 25-30 days, e.g., about 20 days) or about 2-5 weeks (e.g., 2, 3, 4, or 5 weeks) after the first dose of PD-1 inhibitor is administered.

Where the treatment interval comprises multiple doses (e.g., a first and second, and optionally one or more subsequent doses) of a PD-1 inhibitor and a dose of a CAR-expressing cell, in certain embodiments, the dose of the CAR-expressing cell and the first dose of the PD-1 inhibitor are administered simultaneously or concurrently, e.g., within 2 days (e.g., within 2 days, 1 day, 24 hours, 12 hours, 6 hours, 4 hours, 2 hours, or less) of each other. In embodiments, the second dose of the PD-1 inhibitor is administered after either (i) the dose of the CAR-expressing cell or (ii) the first dose of the PD-1 inhibitor, whichever is later. In embodiments, the second dose of the PD-1 inhibitor is administered at least 2 days (e.g., at least 2 days, 3 days, 4 days, 5 days, 6 days, 7 days, 1 week, 2 weeks, 3 weeks, 4 weeks, 5 weeks, or more) after (i) or (ii). In embodiments, a subsequent dose (e.g., third, fourth, or fifth dose, and so on) of the PD-1 inhibitor is administered after the second dose of the PD-1 inhibitor. In embodiments, the subsequent dose of the PD-1 inhibitor is administered at least 2 days (e.g., at least 2 days, 3 days, 4 days, 5 days, 6 days, 7 days, 1 week, 2 weeks, 3 weeks, 4 weeks, 5 weeks, or more) after the second dose of the PD-1 inhibitor. In such embodiments, the treatment interval is initiated upon administration of the first-administered dose and completed upon administration of the second dose (or subsequent dose) of the PD-1 inhibitor.

In other embodiments where the treatment interval comprises multiple doses (e.g., a first and second, and optionally a subsequent dose) of a PD-1 inhibitor and a dose of a CAR-expressing cell, the dose of the CAR-expressing cell and the first dose of the PD-1 inhibitor are administered sequentially. In embodiments, the dose of the CAR-expressing cell is administered after administration of the first dose of the PD-1 inhibitor but before the administration of the second dose of the PD-1 inhibitor. In embodiments, a subsequent dose (e.g., third, fourth, or fifth dose, and so on) of the PD-1 inhibitor is administered after the second dose of the PD-1 inhibitor. In such embodiments, the treatment interval is initiated upon administration of the first dose of the PD-1 inhibitor and completed upon administration of the second dose (or subsequent dose) of the PD-1 inhibitor. In one embodiment, the second dose of the PD-1 inhibitor is administered at least 2 days (e.g., at least 2 days, 3 days, 4 days, 5 days, 6 days, 7 days, 1 week, 2 weeks, 3 weeks, 4 weeks, 5 weeks, or more) after administration of the first dose of the PD-1 inhibitor. In an embodiment, where the PD-1 inhibitor is an inhibitory RNA, e.g., siRNA, the second dose is administered every 2 days to every 2 weeks. In an embodiment, where the PD-1 inhibitor is an antibody molecule, the second dose is administered every 2-3 weeks. In one embodiment, the subsequent dose (e.g., third, fourth, or fifth dose, and so on) of the PD-1 inhibitor is administered at least 2 days (e.g., at least 2 days, 3 days, 4 days, 5 days, 6 days, 7 days, 1 week, 2 weeks, 3 weeks, 4 weeks, 5 weeks, or more) after the second dose of the PD-1 inhibitor. In one embodiment, the dose of the CAR-expressing cell is administered at least 2 days (e.g., at least 2 days, 3 days, 4 days, 5 days, 6 days, 7 days, 1 week, 2 weeks, 3 weeks, 4 weeks, 5 weeks, or more) after administration of the first dose of the PD-1 inhibitor. In one embodiment, the second dose of the PD-1 inhibitor is administered at least 2 days (e.g., at least 2 days, 3 days, 4 days, 5 days, 6 days, 7 days, 1 week, 2 weeks, 3 weeks, 4 weeks, 5 weeks, or more) after administration of the dose of the CAR-expressing cell. In embodiments, the PD-1 inhibitor (e.g., an anti-PD-1 antibody molecule) is administered every 2-3 weeks (e.g., every 2 weeks or every 3 weeks) during the treatment interval.

In other embodiments, the dose of the CAR-expressing cell is administered before administration of the first dose of the PD-1 inhibitor. In such embodiments, the treatment interval is initiated upon administration of the CAR-expressing cell and completed upon administration of the first dose (or subsequent dose) of the PD-1 inhibitor. In embodiments, the first dose of the PD-1 inhibitor is administered at least 2 days (e.g., at least 2 days, at least 3 days, at least 4 days, at least 5 days, at least 6 days, at least 1 week, at least 8 days, at least 9 days, at least 10 days, at least 11 days, at least 12 days, at least 13 days, at least 2 weeks, at least 15 days, at least 16 days, at least 17 days, at least 18 days, at least 19 days, at least 20 days, at least 3 weeks, at least 4 weeks, at least 5 weeks, or more) after administration of the CAR-expressing cell. In some embodiments, administration of the first dose of the PD-1 inhibitor occurs about 5 to about 10 days, e.g., about 8 days, after administration of the CAR-expressing cell. In other embodiments, administration of the first dose of the PD-1 inhibitor occurs about 10 to about 20 days, e.g., about 15 or 16 days, after administration of the CAR-expressing cell. In embodiments, the second dose of the PD-1 inhibitor is administered at least 2 days (e.g., at least 2 days, at least 3 days, at least 4 days, at least 5 days, at least 6 days, at least 1 week, at least 8 days, at least 9 days, at least 10 days, at least 11 days, at least 12 days, at least 13 days, 2 weeks, at least 15 days, at least 16 days, at least 17 days, at least 18 days, at least 19 days, at least 20 days, 3 weeks, 4 weeks, 5 weeks, or more) after administration of the first dose of the PD-1 inhibitor. In embodiments, the second dose of the PD-1 inhibitor is administered at about 2-4 weeks, e.g., 3 weeks after the first dose of the PD-1 inhibitor. In embodiments, the subsequent dose (e.g., third, fourth, or fifth dose, and so on) of the PD-1 inhibitor is administered at least 2 days (e.g., at least 2 days, 3 days, 4 days, 5 days, 6 days, 7 days, 1 week, 2 weeks, 3 weeks, 4 weeks, 5 weeks, or more) after the second dose of the PD-1 inhibitor. In embodiments, the subsequent dose (e.g., third, fourth, or fifth dose, and so on) of the PD-1 inhibitor is administered at about 2-4 weeks, e.g., 3 weeks after the previous dose of the PD-1 inhibitor. In embodiments, the first dose of the PD1 inhibitor is administered at least 2 days (e.g., at least 2 days, 3 days, 4 days, 5 days, 6 days, 7 days, 1 week, 2 weeks, 3 weeks, 4 weeks, 5 weeks, or more) after administration of the CAR-expressing cell.

In some embodiments, the treatment interval comprises one, two or three doses (e.g., a first and second, and a third dose) of a PD-1 inhibitor and a dose of a CAR-expressing cell. In one embodiment, the dose of the CAR-expressing cell and the first dose of the PD-1 inhibitor are administered sequentially. For example, the subject, e.g., a patient, receives one, two or three doses of the PD-1 inhibitor starting post administration of a CAR-expressing cell, e.g., about one week to 4 months, e.g., about 14 days to 2 months, after administration of a dose of CAR-expressing cells.

In one embodiment, any of the treatment intervals described herein can be repeated one or more times, e.g., 1, 2, 3, 4, or 5 more times. In one embodiment, the treatment interval is repeated once, resulting in a treatment regimen comprising two treatment intervals. In an embodiment, the repeated treatment interval is administered at least 1 day, e.g., at least 1 day, at least 2 days, at least 3 days, at least 4 days, at least 5 days, at least 6 days, at least 7 days, at least 2 weeks, at least 1 month, at least 3 months, at least 6 months, at least 1 year or more after the completion of the first or previous treatment interval. In an embodiment, the repeated treatment interval is administered at least 3 days after the completion of the first or previous treatment interval.

In one embodiment, any of the treatment intervals described herein can be followed by one or more, e.g., 1, 2, 3, 4, or 5, subsequent treatment intervals. The one or more subsequent treatment interval is different from the first or previous treatment interval. By way of example, a first treatment interval consisting of a single dose of a PD-1 inhibitor and a single dose of a

CAR-expressing cell is followed by a second treatment interval consisting of multiple doses (e.g., two, three, four, or more doses) of a PD-1 inhibitor and a single dose of a CAR-expressing cell. In one embodiment, the one or more subsequent treatment intervals is administered at least 1 day, e.g., 1 day, 2 days, 3 days, 4 days, 5 days, 6 days, 7 days, or 2 weeks, after the completion of the first or previous treatment interval.

In any of the methods described herein, one or more subsequent doses, e.g., 1, 2, 3, 4, or 5 or more doses, of the PD-1 inhibitor is administered after the completion of one or more treatment intervals. In embodiments where the treatment intervals are repeated or two or more treatment intervals are administered, one or more subsequent doses, e.g., 1, 2, 3, 4, or 5 or more doses, of the PD-1 inhibitor is administered after the completion of one treatment interval and before the initiation of another treatment interval. In one embodiment, a dose of the PD-1 inhibitor is administered every 5 days, 7 days, 2 weeks, 3 weeks, or 4 weeks after the completion of one or more, or each, treatment intervals.

In any of the methods described herein, one or more, e.g., 1, 2, 3, 4, or 5 or more, subsequent doses of the CAR-expressing cell are administered after the completion of one or more treatment intervals. In embodiments where the treatment intervals are repeated or two or more treatment intervals are administered, one or more subsequent doses, e.g., 1, 2, 3, 4, or 5, or more doses, of the CAR-expressing cell is administered after the completion of one treatment interval and before the initiation of another treatment interval. In one embodiment, a dose of the CAR-expressing cell is administered every 2 days, 3 days, 4 days, 5 days, 7 days, 2 weeks, 3 weeks, or 4 weeks after the completion of one or more, or each, treatment intervals.

In one embodiment, the treatment interval comprises a single dose of a CAR-expressing cell that is administered prior to a first dose of a PD-1 inhibitor. In this embodiment, the first dose of the PD-1 inhibitor is administered about 7, about 8, about 9, about 10, about 11, about 12, about 13, about 14, about 15, about 16, about 17, about 18, about 19, about 20, about 25, about 30, or about 35 days after administration of the CAR-expressing cell. In embodiments, a second dose of the PD-1 inhibitor is administered after administration of the first dose of the PD-1 inhibitor. In embodiments, the second dose of the PD-1 inhibitor is administered about 20 days after administration of the first dose of the PD-1 inhibitor, e.g., about 2-4 weeks, e.g., 3 weeks after the first dose of the PD-1 inhibitor. In embodiments, subsequent doses of the PD-1 inhibitor are administered after the second dose of the PD-1 inhibitor, e.g., every 1 week, 2 weeks, 3 weeks, 4 weeks, 5 weeks, 5 days, 7 days, 10 days, 14 days, 20 days, 25 days, 30 days, or 35 days, e.g., about 2-4 weeks, e.g., 3 weeks after the previous dose of the PD-1 inhibitor.

In an embodiment, the method comprises administering radiation to the subject, e.g. prior to administration of the CAR-expressing cell. In embodiments, the total dose of radiation does not exceed a standard dose, e.g. 60 Gy. In embodiments, the total dose of radiation does not exceed 40 Gy. In embodiments, the radiation is administered in one or more fractions, e.g. 2, 3, 4, 5, 6, 7, 8, 9, 10, 11, 12, 13, 14, 15, 16, 17, 18, 19, 20, 21, 22, 23, 24, 25, 26, 27, 28, 29, 30, or more fractions. For example, a total dose of 60 Gy may be delivered in 30 equivalent fractions of 2 Gy each, and a total dose of 40 Gy may be delivered in 15 equivalent fractions of 8/3 Gy each.

In embodiments, the PD-1 inhibitor is administered every 2-4 weeks (e.g., every 2-3 weeks or 3-4 weeks, e.g., every 3 weeks) during the treatment interval). In embodiments, the PD-1 inhibitor is administered at a dose of about 200 mg. In embodiments, the CAR-expressing cell is administered at a dose of about 1.75-5×10⁸ cells per infusion, e.g., about 2×10⁸ cells per infusion or about 5×10⁸ cells per infusion.

In embodiments, the treatment interval comprises a single dose of a CAR-expressing cell (e.g., EGFRvIII CAR-expressing cell) that is administered prior to a first dose of a PD-1 inhibitor, e.g., at least 2 weeks (e.g., 2, 3, 4, 5, 6 weeks or more) prior to the first dose of the PD-1 inhibitor (e.g., about 7, about 8, about 9, about 10, about 11, about 12, about 13, about 14, about 15, about 16, or more days prior to the first dose of the PD-1 inhibitor). In embodiments, the dose of the CAR-expressing cell is administered about 3-4 weeks before the first dose of the PD-1 inhibitor. In embodiments, the PD-1 inhibitor is administered every 2-4 weeks (e.g., every 2-3 weeks or 3-4 weeks, e.g., every 3 weeks) during the treatment interval).

In any of the methods described herein, the subject is administered a single dose of a CAR-expressing cell and a single dose of a PD-1 inhibitor. In one embodiment, the single dose of the CAR-expressing cell is administered at least 2 days, e.g., 2, 3, 4, 5, 6, 7, 8, 9, 10, 14, 15, 16, 17, 18, 20, 25, 30, 35, 40 days, or 2 weeks, 3 weeks, 4 weeks, or more, before administration of the single dose of the PD-1 inhibitor. In embodiments, the single dose of the CAR-expressing cell is administered about 35 days before administration of the PD-1 inhibitor.

In one embodiment, one or more, e.g., 1, 2, 3, 4, or 5, subsequent doses of a CAR-expressing cell are administered to the subject after the initial dose of the CAR-expressing cell. In one embodiment, the one or more subsequent doses of the CAR-expressing cell are administered at least 2 days, e.g., 2, 3, 4, 5, 6, 7, 8, 9, 10, 14, 20, 25, 30, 35, 40 days, or 2 weeks, 3 weeks, 4 weeks, or more, after the previous dose of the CAR-expressing cell. In one embodiment, the one or more subsequent doses of the CAR-expressing cell are administered at least 1 month, e.g., 1, 2, 3, 4, 5, 6, 7, 8, 9, 10, 11, 12, 13, 14, 15, 16, 17, 18, 19, 20 or more months, after the previous dose of the CAR-expressing cell. In one embodiment, the one or more subsequent doses of the CAR-expressing cell are administered at least 5 days after the previous dose of the CAR-expressing cell. In one embodiment, the subject is administered three doses of the CAR-expressing cell per week or one dose every 2 days.

In one embodiment, one or more, e.g., 1, 2, 3, 4, or 5, subsequent doses of PD-1 inhibitor are administered after administration of the single dose of the PD-1 inhibitor. In one embodiment, the one or more subsequent doses of the PD-1 inhibitor are administered at least 5 days, 7 days, 10 days, 14 days, 20 days, 25 days, 30 days, 2 weeks, 3 weeks, 4 weeks, or 5 weeks, e.g., 3 weeks, after the previous dose of PD-1 inhibitor.

In one embodiment, the one or more subsequent doses of the PD-1 inhibitor are administered at least 1, 2, 3, 4, 5, 6, or 7 days, after a dose of the CAR-expressing cell, e.g., the initial dose of the CAR-expressing cell.

In one embodiment, one or more, e.g., 1, 2, 3, 4, or 5, doses of the PD-1 inhibitor is administered prior to the first dose of the CAR-expressing cell.

In one embodiment, one or more, e.g., 1, 2, 3, 4, 5, or 6, doses of the PD-1 inhibitor is administered after the first dose of the CAR-expressing cell, e.g., 2 days, 3 days, 4 days, 5 days, 6 days, 7 days, 2 weeks, 3 weeks, 4 weeks, 5 weeks, 6 weeks, 7 weeks, or 8 weeks after the first dose of the CAR-expressing cell. In one embodiment, the one or more, e.g., 1, 2, 3, 4, or 5, doses of the PD-1 inhibitor is administered after the first dose of the CAR-expressing cells, e.g., 2, 3, 4, 5, 6, 7, 8, 9, 10, 11, 12, 13, 14, 15, 16, 17, 18, 19, or 20 months after the first dose of the CAR-expressing cell.

In one embodiment, one or more, e.g., 1, 2, 3, 4, 5, or 6, doses of the PD-1 inhibitor which is administered after the first dose of the CAR-expressing cell, is administered every 2-3 weeks, e.g., every 2, 3, 4, or 5 weeks, for at least 1 month, e.g., for 1, 2, 3, 4, 5, 6, 7, 8, 9, 10, 11, or 12 months or more. In one embodiment, the one or more doses of the PD-1 inhibitor are administered, e.g., about 2-4 weeks, e.g., 3 weeks after the previous dose of the PD-1 inhibitor, e.g., for up to six doses.

In some embodiments, a dose of CAR-expressing cells (e.g., EGFRvIII CAR-expressing cells) comprises about 10⁴ to about 10⁹ cells/kg, e.g., about 10⁴ to about 10⁵ cells/kg, about 10⁵ to about 10⁶ cells/kg, about 10⁶ to about 10⁷ cells/kg, about 10⁷ to about 10⁸ cells/kg, or about 10⁸ to about 10⁹ cells/kg; or at least about one of: 1×10⁷, 1.5×10⁷, 2×10⁷, 2.5×10⁷, 3×10⁷, 3.5×10⁷, 4×10⁷, 5×10⁷, 1×10⁸, 1.5×10⁸, 2×10⁸, 2.5×10⁸, 3×10⁸, 3.5×10⁸, 4×10⁸, 5×10⁸, 1×10⁹, 2×10⁹, or 5×10⁹ cells. In some embodiments, a dose of CAR-expressing cells (e.g., EGFRvIII CAR-expressing cells) comprises at least about 1-5×10⁷ to 1-5×10⁸ CAR-expressing cells. In other embodiments, the subject is administered about 1.75-5×10⁸ CAR-expressing cells (e.g., EGFRvIII CAR-expressing cells).

In embodiments, the CAR-expressing cells (e.g., EGFRvIII CAR-expressing cells) are administered to the subject according to a dosing regimen comprising a total dose of cells administered to the subject by dose fractionation, e.g., one, two, three or more separate administration of a partial dose. In embodiments, a first percentage of the total dose is administered on a first day of treatment, a second percentage of the total dose is administered on a subsequent (e.g., second, third, fourth, fifth, sixth, or seventh or later) day of treatment, and optionally, a third percentage (e.g., the remaining percentage) of the total dose is administered on a yet subsequent (e.g., third, fourth, fifth, sixth, seventh, eighth, ninth, tenth, or later) day of treatment. For example, 10% of the total dose of cells is delivered on the first day, 30% of the total dose of cells is delivered on the second day, and the remaining 60% of the total dose of cells is delivered on the third day of treatment. For example, a total cell dose includes 1.75 to 5×10⁸ CAR-expressing cells (e.g., EGFRvIII CAR-expressing cells).

In any of the administration regimens described herein, a dose of a PD-1 inhibitor, e.g., an anti-PD-1 antibody molecule described herein (e.g., pembrolizumab, nivolumab, PDR001, or an anti-PD-1 antibody molecule provided in Table 6), comprises about 1 to 30 mg/kg, e.g., about 1 to 20 mg/kg, about 2 to 15 mg/kg, about 5 to 25 mg/kg, about 10 to 20 mg/kg, about 1 to 5 mg/kg, about 2 mg/kg, about 3 mg/kg, or about 10 mg/kg. In one embodiment, the dose is about 10 to 20 mg/kg. In one embodiment, the dose is about 1 to 5 mg/kg. In one embodiment, the dose is less than 5 mg/kg, less than 4 mg/kg, less than 3 mg/kg, less than 2 mg/kg, or less than 1 mg/kg. In one embodiment, the dose is about 2 mg/kg.

In embodiments, in any of the administration regimens described herein, the dose of the PD-1 inhibitor is administered every 1-4 weeks, e.g., every week, every 2 weeks, every 3 weeks, or every 4 weeks.

In certain embodiments, the anti-PD-1 antibody molecule (e.g., pembrolizumab, nivolumab, PDR001, or an anti-PD-1 antibody molecule provided in Table 6) is administered by injection (e.g., subcutaneously or intravenously) at a dose of about 1 to 30 mg/kg, e.g., about 1 to 20 mg/kg, about 2 to 15 mg/kg, about 5 to 25 mg/kg, about 10 to 20 mg/kg, about 1 to 5 mg/kg, about 3 mg/kg, or about 2 mg/kg. The dosing schedule can vary from e.g., once a week to once every 2, 3, or 4 weeks. In one embodiment, the anti-PD-1 antibody molecule is administered at a dose from about 10 to 20 mg/kg every other week. In one embodiment, the dose is about 1 to 5 mg/kg every 2 weeks, every 3 weeks, or every 4 weeks. In one embodiment, the dose is less than 5 mg/kg, less than 4 mg/kg, less than 3 mg/kg, less than 2 mg/kg, or less than 1 mg/kg, every 2 weeks, every 3 weeks, or every 4 weeks. In one embodiment, the dose is about 2 mg/kg, every 2 weeks, every 3 weeks, or every 4 weeks.

In some embodiments, the dose of a PD-1 inhibitor, e.g., an anti-PD-1 antibody molecule (e.g., pembrolizumab, nivolumab, PDR001 or an anti-PD-1 antibody molecule provided in Table 6), is a flat dose. In some embodiments, the anti-PD-1 antibody molecule is administered by injection (e.g., subcutaneously or intravenously) at a dose (e.g., a flat dose) of about 200 mg to 500 mg, e.g., about 250 mg to 450 mg, about 300 mg to 400 mg, about 250 mg to 350 mg, about 350 mg to 450 mg, or about 200 mg, about 300 mg or about 400 mg. The dosing schedule (e.g., flat dosing schedule) can vary from, e.g., once a week to once every 2, 3, 4, 5, or 6 weeks. In one embodiment, the anti-PD-1 antibody molecule is administered at a dose from about 200 mg once every three weeks or once every four weeks. In one embodiment, the anti-PD-1 antibody molecule is administered at a dose from about 300 mg to 400 mg once every three weeks or once every four weeks. In one embodiment, the anti-PD-1 antibody molecule is administered at a dose from about 300 mg once every three weeks, e.g., via i.v. infusion. In one embodiment, the anti-PD-1 antibody molecule is administered at a dose from about 200 mg once every three weeks, e.g., via i.v. infusion. In one embodiment, the anti-PD-1 antibody molecule is administered at a dose from about 400 mg once every four weeks, e.g., via i.v. infusion. In one embodiment, the anti-PD-1 antibody molecule is administered at a dose from about 300 mg once every four weeks, e.g., via i.v. infusion. In one embodiment, the anti-PD-1 antibody molecule is administered at a dose from about 400 mg once every three weeks, e.g., via i.v. infusion.

In one embodiment, the PD-1 inhibitor is pembrolizumab administered at 200 mg every three weeks for up to six doses. In some embodiments, the PD-1 inhibitor is pembrolizumab administered at 300 mg every three weeks for up to six doses.

In one embodiment, the PD-1 inhibitor is selected from the group consisting of Nivolumab, Pembrolizumab, Pidilizumab, PDR001, AMP 514, AMP-224, and any anti-PD-1 antibody molecule provided in Table 6.

In some embodiments, the disclosure provides a method of treating a subject having a disease associated with expression of EGFRvIII, e.g. glioblastoma (GBM) (e.g., IDH-wildtype GBM, IDH-mutant GBM, or MGMT-unmethylated GBM). The method comprises administering to the subject an effective number of a population of cells that express a CAR molecule that binds EGFRvIII, e.g., an EGFRvIII CAR (“EGFRvIII CAR therapy”) as described herein, in combination with a PD1 inhibitor, e.g., an anti-PD1 antibody as described herein. In some embodiments, the EGFRvIII CAR therapy is administered prior to, simultaneously with or after the PD-1 inhibitor. In one embodiment, the EGFRvIII CAR therapy is administered prior to the PD-1 inhibitor. For example, one or more doses of the PD-1 inhibitor can be administered post-EGFRvIII CAR therapy (e.g., starting 5 days to 4 months, e.g., 10 day to 3 months, e.g., 14 days to 2 months post-EGFRvIII CAR therapy). In some embodiments, the combination of the EGFRvIII CAR therapy and PD-1 inhibitor therapy is repeated.

In one embodiment of the therapy comprising the EGFRvIII CAR-expressing cell and the PD1 inhibitor, the EGFRvIII CAR therapy comprises one or more treatments with cells that express an EGFRvIII CAR as described herein. In embodiments, the EGFRvIII CAR molecule comprises an antigen binding domain that binds specifically to EGFRvIII, e.g., as described herein. In embodiments, the EGFRvIII CAR and PD-1 inhibitor therapies are administered at a dosage described herein.

In some embodiments, the EGFRvIII CAR (or a nucleic acid encoding it) comprises a sequence set out in any of Table 2.

In another embodiment of the therapy comprising the EGFRvIII CAR-expressing cell and the PD1 inhibitor, the EGFRvIII CAR therapy comprises one or more treatments with cells that express a humanized EGFRvIII CAR, e.g., a humanized EGFRvIII CAR according to Table 2.

In some embodiments, the CAR molecule comprises one, two, and/or three CDRs from the heavy chain variable region and/or one, two, and/or three CDRs from the light chain variable region of the murine or humanized EGFRvIII CAR of Table 2.

In some embodiments of the therapy comprising the EGFRvIII CAR-expressing cell and the PD1 inhibitor, the PD-1 inhibitor is an antibody to PD-1. In some embodiments, the PD-1 inhibitor is chosen from pembrolizumab, nivolumab, PDR001 (e.g., an antibody molecule of Table 6), MEDI-0680 (AMP-514), AMP-224, REGN-2810, or BGB-A317.

In one embodiment of the therapy comprising the EGFRvIII CAR-expressing cell and the PD1 inhibitor, the PD-1 inhibitor is pembrolizumab. In one embodiment, the antibody molecule includes:

(i) a heavy chain variable (VH) region comprising the VHCDR1 amino acid sequence of SEQ ID NO: 503; the VHCDR2 amino acid sequence of SEQ ID NO: 504; and the VHCDR3 amino acid sequence of SEQ ID NO: 505; and

(ii) a light chain variable (VL) region comprising the VLCDR1 amino acid sequence of SEQ ID NO: 500; the VLCDR2 amino acid sequence of SEQ ID NO: 501; and the VLCDR3 amino acid sequence of SEQ ID NO: 502, or an amino acid sequence at least 85%, 90%, 95% identical or higher.

In another embodiment of the therapy comprising the EGFRvIII CAR-expressing cell and the PD1 inhibitor, the PD-1 inhibitor, e.g., the anti-PD-1 antibody molecule, includes at least one, two, three, four, five or six CDRs (or collectively all of the CDRs) from a heavy and light chain variable region from an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 1 of US 2015/0210769, or in Table 6 herein; or encoded by the nucleotide sequence in Table 6 herein, or a sequence substantially identical (e.g., at least 80%, 85%, 90%, 92%, 95%, 97%, 98%, 99% or higher identical) to any of the aforesaid sequences; or closely related CDRs, e.g., CDRs which are identical or which have at least one amino acid alteration, but not more than two, three or four alterations (e.g., substitutions, deletions, or insertions, e.g., conservative substitutions).

In yet another embodiment of the therapy comprising the EGFRvIII CAR-expressing cell and the PD1 inhibitor, the PD-1 inhibitor, e.g., the anti-PD-1 antibody molecule, comprises at least one, two, three or four variable regions from an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 1 of US 2015/0210769, or in Table 6 herein encoded by the nucleotide sequence in Table 6 herein, or a sequence substantially identical (e.g., at least 80%, 85%, 90%, 92%, 95%, 97%, 98%, 99% or higher identical) to any of the aforesaid sequences.

In embodiments, the PD-1 inhibitor, e.g., anti-PD-1 antibody molecule, is PDR-001, which contains the variable light chain and variable heavy chain amino acid sequences of BAP049-Clone-E, as described in Table 6.

In one embodiment of the therapy comprising the EGFRvIII CAR-expressing cell and the PD1 inhibitor, the PD-1 inhibitor, e.g., pembrolizumab, is administered post-EGFRvIII CAR therapy (e.g., starting 5 days to 4 months, e.g., 10 day to 3 months, e.g., 14 days to 2 months post-EGFRvIII CAR therapy). In embodiments, administration of the therapy is to a subject with GBM, e.g., relapsed or refractory GBM.

In yet another embodiment of the therapy comprising the EGFRvIII CAR-expressing cell and the PD1 inhibitor, the GBM is IDH-wildtype GBM, IDH-mutant GBM, or MGMT-unmethylated GBM, e.g., relapsed or refractory IDH-wildtype GBM, IDH-mutant GBM, or MGMT-unmethylated GBM. In one embodiment, the subject has a GBM, e.g., IDH-wildtype GBM, IDH-mutant GBM, or MGMT-unmethylated GBM, and may not respond to the CAR T therapy or may relapse, e.g., due to poor CAR T cell persistence. In one embodiment of the EGFRvIII CAR therapy-PD1 inhibitor therapy, the subject shows an improved therapeutic outcome, e.g., the subject achieves one or more of partial remission, complete remission, or prolonged CAR T cell persistence, in response to the EGFRvIII CAR therapy-PD1 inhibitor therapy, e.g., one or more cycles of the EGFRvIII CAR therapy-PD1 inhibitor therapy.

In one embodiment of the therapy comprising the EGFRvIII CAR-expressing cell and the PD1 inhibitor, prior to administration of the PD-1 inhibitor, the subject has relapsed or refractory IDH-wildtype GBM, IDH-mutant GBM, or MGMT-unmethylated GBM to a prior treatment with an EGFRvIII CAR therapy, e.g., a prior treatment with one or both EGFRvIII CAR therapy or the standard of care (radiation and TMZ). In some embodiments, the subject shows decreased or poor CAR T cell persistence.

In embodiments, the further administration of the combination therapy results in an improved therapeutic outcome, e.g., the subject achieves one or more of partial remission, complete remission, or a prolonged CAR T cell persistence. In an embodiment, the administration of the combination therapy results in prolonged persistence of a CAR T cell, e.g., an EGFRvIII CAR-expressing cell. In some embodiments, the subject after treatment with the combination disclosed herein has one or more of: (i) a decreased risk of relapse, (ii) delayed timing of the onset of relapse, or (iii) decreased severity of relapse, e.g., compared to a subject treated with EGFRvIII CAR therapy alone and/or the standard of care (radiation and TMZ). In an embodiment, administration of the combination therapy results in an objective clinical response.

In an embodiment, the subject, e.g., a subject showing relapse after an EGFRvIII CAR therapy, is eligible to receive repeat administration of an EGFRvIII CAR therapy, e.g., a second, third or fourth dose. In an embodiment, the subject is eligible to receive a repeat administration of an EGFRvIII CAR therapy, e.g., a second, third or fourth dose, along with a PD-1 inhibitor. In an embodiment, a subject showing low persistence of EGFRvIII CAR therapy after a first administration of an EGFRvIII CAR therapy is eligible to receive a repeat administration of an EGFRvIII CAR therapy, e.g., a second, third or fourth dose, along with a PD-1 inhibitor.

Optionally, the subject has, or is identified as having, at least 5%, 6%, 7%, 8%, 9%, 10%, 20%, 30%, 40%, 50%, 60%, 70%, 80%, or 90% of cancer cells, e.g., GBM cells, which are CD3+/PD1+.

In another aspect, the disclosure features a composition (e.g., one or more dosage formulations, combinations, or one or more pharmaceutical compositions) comprising a cell expressing a CAR (e.g., EGFRvIII CAR) described herein and a PD-1 inhibitor described herein. In one embodiment, the CAR (e.g., EGFRvIII CAR) comprises an antigen binding domain (e.g., EGFRvIII antigen binding domain), a transmembrane domain, and an intracellular signaling domain, as described herein. In one embodiment, the EGFRvIII CAR comprises an EGFRvIII antigen binding domain listed in Table 2. In one embodiment, the PD-1 inhibitor comprises an antibody molecule, a small molecule, a polypeptide, e.g., a fusion protein, or an inhibitory nucleic acid, e.g., a siRNA or shRNA. In one embodiment, the PD-1 inhibitor comprises an antibody molecule, e.g., pembrolizumab, nivolumab, PDR001 or an antibody molecule listed in Table 6. The CAR-expressing cell and the PD-1 inhibitor can be in the same or different formulation or pharmaceutical composition.

In another aspect, the disclosure features a composition (e.g., one or more dosage formulations, combinations, or one or more pharmaceutical compositions) comprising a cell expressing a CAR (e.g., EGFRvIII CAR) described herein and a PD-1 inhibitor described herein, for use in a method of treating a disease (e.g., cancer), e.g., disease associated with expression of EGFRvIII, e.g., a cancer described herein. In one embodiment, the CAR (e.g., EGFRvIII CAR) comprises an antigen binding domain (e.g., EGFRvIII antigen binding domain), a transmembrane domain, and an intracellular signaling domain, as described herein. In one embodiment, the EGFRvIII CAR comprises an EGFRvIII antigen binding domain listed in Table 2. In one embodiment, the PD-1 inhibitor comprises an antibody molecule, a small molecule, a polypeptide, e.g., a fusion protein, or an inhibitory nucleic acid, e.g., a siRNA or shRNA. In one embodiment, the PD-1 inhibitor comprises an antibody molecule, e.g., pembrolizumab, nivolumab, PDR001, or an antibody molecule listed in Table 6. The CAR-expressing cell and the PD-1 inhibitor can be in the same or different formulation or pharmaceutical composition.

### PD-1 Inhibitors

Provided herein are PD-1 inhibitors for use in any of the methods or compositions described herein. In any of the methods or compositions described herein, the PD-1 inhibitor comprises an antibody molecule, a small molecule, a polypeptide, e.g., a fusion protein, or an inhibitory nucleic acid, e.g., a siRNA or shRNA.

In one embodiment, the PD-1 inhibitor is characterized by one or more of the following: inhibits or reduces PD-1 expression, e.g., transcription or translation of PD-1; inhibits or reduces PD-1 activity, e.g., inhibits or reduces binding of PD-1 to its ligand, e.g., PD-L1; or binds to PD-1 or its ligand, e.g., PD-L1.

In one embodiment, the PD-1 inhibitor is an antibody molecule.

In one embodiment, the PD-1 inhibitor comprises an anti-PD-1 antibody molecule comprising a heavy chain complementary determining region 1 (HC CDR1), a heavy chain complementary determining region 2 (HC CDR2), and a heavy chain complementary determining region 3 (HC CDR3) of any PD-1 antibody molecule amino acid sequence listed in Table 6; and/or a light chain complementary determining region 1 (LC CDR1), a light chain complementary determining region 2 (LC CDR2), and a light chain complementary determining region 3 (LC CDR3) of any PD-1 antibody molecule amino acid sequence listed in Table 6.

In one embodiment, the anti-PD1 antibody molecule comprises a HC CDR1 amino acid sequence chosen from SEQ ID NO: 137 or 140, a HC CDR2 amino acid sequence of SEQ ID NO: 138 or 141, and a HC CDR3 amino acid sequence of SEQ ID NO: 139; and/or a LC CDR1 amino acid sequence of SEQ ID NO: 146 or 149, a LC CDR2 amino acid sequence of SEQ ID NO: 147 or 150, and a LC CDR3 amino acid sequence of SEQ ID NO: 148, 151, 166, or 167. In one embodiment, the anti-PD-1 antibody comprises a HC CDR1 amino acid sequence chosen from SEQ ID NO: 137 or 140, a HC CDR2 amino acid sequence of SEQ ID NO: 138 or 141, and a HC CDR3 amino acid sequence of SEQ ID NO: 139; and/or a LC CDR1 amino acid sequence of SEQ ID NO: 146 or 149, a LC CDR2 amino acid sequence of SEQ ID NO: 147 or 150, and a LC CDR3 amino acid sequence of SEQ ID NO: 166 or 167.

In one embodiment, the anti-PD-1 antibody molecule comprises a heavy chain variable region comprising the amino acid sequence of any heavy chain variable region listed in Table 6, e.g., SEQ ID NOs: 142, 144, 154, 158, 154, 158, 172, 184, 216, or 220. In one embodiment, the anti-PD-1 antibody molecule comprises a heavy chain variable region comprising the amino acid sequence having at least one, two or three modifications but not more than 30, 20 or 10 modifications to the amino acid sequence of any heavy chain variable region provided in Table 6, e.g., SEQ ID NOs: 142, 144, 154, 158, 154, 158, 172, 184, 216, or 220. In one embodiment, the anti-PD-1 antibody molecule comprises a heavy chain variable region comprising an amino acid sequence at least 95% identical (e.g., with 95-99% identity) to the amino acid sequence of any heavy chain variable region provided in Table 6, e.g., SEQ ID NOs: 142, 144, 154, 158, 154, 158, 172, 184, 216, or 220.

In one embodiment, the anti-PD-1 antibody molecule comprises a heavy chain comprising the amino acid sequence of any heavy chain listed in Table 6, e.g., SEQ ID NOs:156, 160, 174, 186, 218, 222, 225, or 236. In one embodiment, the anti-PD-1 antibody molecule comprises a heavy chain comprising the amino acid sequence having at least one, two or three modifications but not more than 30, 20 or 10 modifications to any heavy chain listed in Table 6, e.g., SEQ ID NOs: 156, 160, 174, 186, 218, 222, 225, or 236. In one embodiment, the anti-PD-1 antibody molecule comprises a heavy chain comprising an amino acid sequence with 95-99% identity to the amino acid sequence of any heavy chain listed in Table 6, e.g., SEQ ID NOs: 156, 160, 174, 186, 218, 222, 225, or 236.

In one embodiment, the anti-PD-1 antibody molecule comprises a light chain variable region comprising the amino acid sequence of any light chain variable region listed in Table 6, e.g., SEQ ID NOs: 152, 162, 168, 176, 180, 188, 192, 196, 200, 204, 208, or 212. In one embodiment, the anti-PD-1 antibody molecule comprises a light chain variable region comprising the amino acid sequence having at least one, two or three modifications but not more than 30, 20 or 10 modifications to the amino acid sequence of any light chain variable region provided in Table 6, e.g., SEQ ID NOs: 152, 162, 168, 176, 180, 188, 192, 196, 200, 204, 208, or 212. In one embodiment, the anti-PD-1 antibody molecule comprises a light chain variable region comprising an amino acid sequence at least 95% identical (e.g., with 95-99% identity) to the amino acid sequence of any light chain variable region provided in Table 6, e.g., SEQ ID NOs: 152, 162, 168, 176, 180, 188, 192, 196, 200, 204, 208, or 212.

In one embodiment, the anti-PD-1 antibody molecule comprises a light chain comprising the amino acid sequence of any light chain listed in Table 6, e.g., SEQ ID NOs: 164, 170, 178, 182, 190, 194, 198, 202, 206, 210, or 214. In one embodiment, the anti-PD-1 antibody molecule comprises a light chain comprising the amino acid sequence having at least one, two or three modifications but not more than 30, 20 or 10 modifications to any light chain listed in Table 6, e.g., SEQ ID NOs: 164, 170, 178, 182, 190, 194, 198, 202, 206, 210, or 214. In one embodiment, the anti-PD-1 antibody molecule comprises a light chain comprising an amino acid sequence at least 95% identical (e.g., with 95-99% identity) to the amino acid sequence to any any light chain listed in Table 6, e.g., SEQ ID NOs: 164, 170, 178, 182, 190, 194, 198, 202, 206, 210, or 214.

In one embodiment, the anti-PD-1 antibody molecule comprises:
i) a heavy chain comprising the amino acid sequence of SEQ ID NO: 144 and a light chain comprising the amino acid sequence of SEQ ID NO: 152;
ii) a heavy chain comprising the amino acid sequence of SEQ ID NO: 156 or 160 and a light chain comprising the amino acid sequence of SEQ ID NO: 164;
iii) a heavy chain comprising the amino acid sequence of SEQ ID NO: 156 or 160 and a light chain comprising the amino acid sequence of SEQ ID NO: 170.
iv) a heavy chain comprising the amino acid sequence of SEQ ID NO: 174 and a light chain comprising the amino acid sequence of SEQ ID NO: 178;
v) a heavy chain comprising the amino acid sequence of SEQ ID NO: 174 and a light chain comprising the amino acid sequence of SEQ ID NO: 182;
vi) a heavy chain comprising the amino acid sequence of SEQ ID NO: 186 and a light chain comprising the amino acid sequence of SEQ ID NO: 182;
vii) a heavy chain comprising the amino acid sequence of SEQ ID NO: 186 and a light chain comprising the amino acid sequence of SEQ ID NO: 190;
viii) a heavy chain comprising the amino acid sequence of SEQ ID NO: 174 and a light chain comprising the amino acid sequence of SEQ ID NO: 190;
ix) a heavy chain comprising the amino acid sequence of SEQ ID NO: 174 and a light chain comprising the amino acid sequence of SEQ ID NO: 194;
x) a heavy chain comprising the amino acid sequence of SEQ ID NO: 174 and a light chain comprising the amino acid sequence of SEQ ID NO: 198;
xi) a heavy chain comprising the amino acid sequence of SEQ ID NO: 186 and a light chain comprising the amino acid sequence of SEQ ID NO: 202;
xii) a heavy chain comprising the amino acid sequence of SEQ ID NO: 174 and a light chain comprising the amino acid sequence of SEQ ID NO: 202;
xiii) a heavy chain comprising the amino acid sequence of SEQ ID NO: 186 and a light chain comprising the amino acid sequence of SEQ ID NO: 206;
xiv) a heavy chain comprising the amino acid sequence of SEQ ID NO: 174 and a light chain comprising the amino acid sequence of SEQ ID NO: 206;
xv) a heavy chain comprising the amino acid sequence of SEQ ID NO: 174 and a light chain comprising the amino acid sequence of SEQ ID NO: 210;
xvi) a heavy chain comprising the amino acid sequence of SEQ ID NO: 174 and a light chain comprising the amino acid sequence of SEQ ID NO: 214;
xvii) a heavy chain comprising the amino acid sequence of SEQ ID NO: 218 and a light chain comprising the amino acid sequence of SEQ ID NO: 206;
xviii) a heavy chain comprising the amino acid sequence of SEQ ID NO: 218 and a light chain comprising the amino acid sequence of SEQ ID NO: 202;
xix) a heavy chain comprising the amino acid sequence of SEQ ID NO: 222 and a light chain comprising the amino acid sequence of SEQ ID NO: 202;
xx) a heavy chain comprising the amino acid sequence of SEQ ID NO: 225 and a light chain comprising the amino acid sequence of SEQ ID NO: 178;
xxi) a heavy chain comprising the amino acid sequence of SEQ ID NO: 225 and a light chain comprising the amino acid sequence of SEQ ID NO: 190;
xxii) a heavy chain comprising the amino acid sequence of SEQ ID NO: 225 and a light chain comprising the amino acid sequence of SEQ ID NO: 202;
xxiii) a heavy chain comprising the amino acid sequence of SEQ ID NO: 236 and a light chain comprising the amino acid sequence of SEQ ID NO: 206; or
xxiv) a heavy chain comprising the amino acid sequence of SEQ ID NO: 225 and a light chain comprising the amino acid sequence of SEQ ID NO: 206.

In one embodiment, the anti-PD-1 antibody molecule comprises:
i) a heavy chain variable domain comprising the amino acid sequence of SEQ ID NO: 172 and a light chain variable domain comprising the amino acid sequence of SEQ ID NO: 204;
ii) a heavy chain variable domain comprising the amino acid sequence of SEQ ID NO: 142 or 144 and a light chain variable domain comprising the amino acid sequence of SEQ ID NO: 152;
iii) a heavy chain variable domain comprising the amino acid sequence of SEQ ID NO: 154 or 158 and a light chain variable domain comprising the amino acid sequence of SEQ ID NO: 162;
iv) a heavy chain variable domain comprising the amino acid sequence of SEQ ID NO: 154 or 158 and a light chain variable domain comprising the amino acid sequence of SEQ ID NO: 168;
v) a heavy chain variable domain comprising the amino acid sequence of SEQ ID NO: 172 and a light chain variable domain comprising the amino acid sequence of SEQ ID NO: 176;
vi) a heavy chain variable domain comprising the amino acid sequence of SEQ ID NO: 172 and a light chain variable domain comprising the amino acid sequence of SEQ ID NO: 180;
vii) a heavy chain variable domain comprising the amino acid sequence of SEQ ID NO: 184 and a light chain variable domain comprising the amino acid sequence of SEQ ID NO: 180;
viii) a heavy chain variable domain comprising the amino acid sequence of SEQ ID NO: 184 and a light chain variable domain comprising the amino acid sequence of SEQ ID NO: 188;
ix) a heavy chain variable domain comprising the amino acid sequence of SEQ ID NO: 172 and a light chain variable domain comprising the amino acid sequence of SEQ ID NO: 188;
x) a heavy chain variable domain comprising the amino acid sequence of SEQ ID NO: 172 and a light chain variable domain comprising the amino acid sequence of SEQ ID NO: 192;
xi) a heavy chain variable domain comprising the amino acid sequence of SEQ ID NO: 172 and a light chain variable domain comprising the amino acid sequence of SEQ ID NO: 196;
xii) a heavy chain variable domain comprising the amino acid sequence of SEQ ID NO: 184 and a light chain variable domain comprising the amino acid sequence of SEQ ID NO: 200;
xiii) a heavy chain variable domain comprising the amino acid sequence of SEQ ID NO: 172 and a light chain variable domain comprising the amino acid sequence of SEQ ID NO: 200;
xiv) a heavy chain variable domain comprising the amino acid sequence of SEQ ID NO: 184 and a light chain variable domain comprising the amino acid sequence of SEQ ID NO: 204;
xv) a heavy chain variable domain comprising the amino acid sequence of SEQ ID NO: 172 and a light chain variable domain comprising the amino acid sequence of SEQ ID NO: 204;
xvi) a heavy chain variable domain comprising the amino acid sequence of SEQ ID NO: 172 and a light chain variable domain comprising the amino acid sequence of SEQ ID NO: 208;
xvii) a heavy chain variable domain comprising the amino acid sequence of SEQ ID NO: 172 and a light chain variable domain comprising the amino acid sequence of SEQ ID NO: 212;
xviii) a heavy chain variable domain comprising the amino acid sequence of SEQ ID NO: 216 and a light chain variable domain comprising the amino acid sequence of SEQ ID NO: 204;
xix) a heavy chain variable domain comprising the amino acid sequence of SEQ ID NO: 216 and a light chain variable domain comprising the amino acid sequence of SEQ ID NO: 200;
xx) a heavy chain variable domain comprising the amino acid sequence of SEQ ID NO: 220 and a light chain variable domain comprising the amino acid sequence of SEQ ID NO: 200;
xxi) a heavy chain variable domain comprising the amino acid sequence of SEQ ID NO: 172 and a light chain variable domain comprising the amino acid sequence of SEQ ID NO: 176;

xxii) a heavy chain variable domain comprising the amino acid sequence of SEQ ID NO: 172 and a light chain variable domain comprising the amino acid sequence of SEQ ID NO: 188;

xxiii) a heavy chain variable domain comprising the amino acid sequence of SEQ ID NO: 172 and a light chain variable domain comprising the amino acid sequence of SEQ ID NO: 200; or

xxiv) a heavy chain variable domain comprising the amino acid sequence of SEQ ID NO: 184 and a light chain variable domain comprising the amino acid sequence of SEQ ID NO: 204.

In one embodiment, the anti-PD-1 antibody molecule includes at least one or two heavy chain variable domain (optionally including a constant region), at least one or two light chain variable domain (optionally including a constant region), or both, comprising the amino acid sequence of BAP049-Clone-A, BAP049-Clone-B, BAP049-Clone-C, BAP049-Clone-D, or BAP049-Clone-E; or as described in Table 1 of US 2015/0210769, or in Table 6 herein, or encoded by the nucleotide sequence in Table 6; or a sequence substantially identical (e.g., at least 80%, 85%, 90%, 92%, 95%, 97%, 98%, 99% or higher identical) to any of the aforesaid sequences. The anti-PD-1 antibody molecule, optionally, comprises a leader sequence from a heavy chain, a light chain, or both, as shown in Table 4 of US 2015/0210769; or a sequence substantially identical thereto.

In yet another embodiment, the anti-PD-1 antibody molecule includes at least one, two, or three complementarity determining regions (CDRs) from a heavy chain variable region and/or a light chain variable region of an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 6, or encoded by the nucleotide sequence in Table 6; or a sequence substantially identical (e.g., at least 80%, 85%, 90%, 92%, 95%, 97%, 98%, 99% or higher identical) to any of the aforesaid sequences.

In yet another embodiment, the anti-PD-1 antibody molecule includes at least one, two, or three CDRs (or collectively all of the CDRs) from a heavy chain variable region comprising an amino acid sequence shown in Table 1 of US 2015/0210769, or in Table 6 herein, or encoded by a nucleotide sequence shown in Table 6. In one embodiment, one or more of the CDRs (or collectively all of the CDRs) have one, two, three, four, five, six or more changes, e.g., amino acid substitutions or deletions, relative to the amino acid sequence shown in Table 6, or encoded by a nucleotide sequence shown in Table 6.

In yet another embodiment, the anti-PD-1 antibody molecule includes at least one, two, or three CDRs (or collectively all of the CDRs) from a light chain variable region comprising an amino acid sequence shown in Table 1 of US 2015/0210769, or in Table 6 herein, or encoded by a nucleotide sequence shown in Table 6. In one embodiment, one or more of the CDRs (or collectively all of the CDRs) have one, two, three, four, five, six or more changes, e.g., amino acid substitutions or deletions, relative to the amino acid sequence shown in Table 6, or encoded by a nucleotide sequence shown in Table 6. In certain embodiments, the anti-PD-1 antibody molecule includes a substitution in a light chain CDR, e.g., one or more substitutions in a CDR1, CDR2 and/or CDR3 of the light chain. In one embodiment, the anti-PD-1 antibody molecule includes a substitution in the light chain CDR3 at position 102 of the light variable region, e.g., a substitution of a cysteine to tyrosine, or a cysteine to serine residue, at position 102 of the light variable region according to Table 6 (e.g., SEQ ID NO: 152 or 162 for murine or chimeric, unmodified; or any of SEQ ID NOs: 168, 176, 180, 188, 192, 196, 200, 204, 208, or 212 for a modified sequence).

In another embodiment, the anti-PD-1 antibody molecule includes at least one, two, three, four, five or six CDRs (or collectively all of the CDRs) from a heavy and light chain variable region comprising an amino acid sequence shown in Table 1 of US 2015/0210769, or in Table 6 herein, or encoded by a nucleotide sequence shown in Table 6. In one embodiment, one or more of the CDRs (or collectively all of the CDRs) have one, two, three, four, five, six or more changes, e.g., amino acid substitutions or deletions, relative to the amino acid sequence shown in Table 6, or encoded by a nucleotide sequence shown in Table 6.

In one embodiment, the anti-PD-1 antibody molecule includes:

(a) a heavy chain variable region (VH) comprising a VHCDR1 amino acid sequence of SEQ ID NO: 140, a VHCDR2 amino acid sequence of SEQ ID NO: 141, and a VHCDR3 amino acid sequence of SEQ ID NO: 139; and a light chain variable region (VL) comprising a VLCDR1 amino acid sequence of SEQ ID NO: 149, a VLCDR2 amino acid sequence of SEQ ID NO: 150, and a VLCDR3 amino acid sequence of SEQ ID NO: 167, each disclosed in Table 1 of US 2015/0210769, or in Table 6 herein;

(b) a VH comprising a VHCDR1 amino acid sequence chosen from SEQ ID NO: 137; a VHCDR2 amino acid sequence of SEQ ID NO: 138; and a VHCDR3 amino acid sequence of SEQ ID NO: 139; and a VL comprising a VLCDR1 amino acid sequence of SEQ ID NO: 146, a VLCDR2 amino acid sequence of SEQ ID NO: 147, and a VLCDR3 amino acid sequence of SEQ ID NO: 166, each disclosed in Table 1 of US 2015/0210769, or in Table 6 herein;

(c) a VH comprising a VHCDR1 amino acid sequence of SEQ ID NO: 286, a VHCDR2 amino acid sequence of SEQ ID NO: 141, and a VHCDR3 amino acid sequence of SEQ ID NO: 139; and a VL comprising a VLCDR1 amino acid sequence of SEQ ID NO: 149, a VLCDR2 amino acid sequence of SEQ ID NO: 150, and a VLCDR3 amino acid sequence of SEQ ID NO: 167, each disclosed in Table 1 of US 2015/0210769, or in Table 6 herein; or

(d) a VH comprising a VHCDR1 amino acid sequence of SEQ ID NO: 286; a VHCDR2 amino acid sequence of SEQ ID NO: 138; and a VHCDR3 amino acid sequence of SEQ ID NO: 139; and a VL comprising a VLCDR1 amino acid sequence of SEQ ID NO: 146, a VLCDR2 amino acid sequence of SEQ ID NO: 147, and a VLCDR3 amino acid sequence of SEQ ID NO: 166, each disclosed in Table 1 of US 2015/0210769, or in Table 6 herein.

In the combinations herein below, in another embodiment, the anti-PD-1 antibody molecule comprises (i) a heavy chain variable region (VH) comprising a VHCDR1 amino acid sequence chosen from SEQ ID NO: 137, SEQ ID NO: 140, or SEQ ID NO: 286; a VHCDR2 amino acid sequence of SEQ ID NO: 138 or SEQ ID NO: 141; and a VHCDR3 amino acid sequence of SEQ ID NO: 139; and (ii) a light chain variable region (VL) comprising a VLCDR1 amino acid sequence of SEQ ID NO: 146 or SEQ ID NO: 149, a VLCDR2 amino acid sequence of SEQ ID NO: 147 or SEQ ID NO: 150, and a VLCDR3 amino acid sequence of SEQ ID NO: 166 or SEQ ID NO: 167, each disclosed in Table 1 of US 2015/0210769, or in Table 6 herein.

In embodiments, the PD-1 inhibitor, e.g., anti-PD-1 antibody molecule, is PDR-001, which contains the variable light chain and variable heavy chain amino acid sequences of BAP049-Clone-E, as described in Table 6. In one embodiment, the anti-PD-1 antibody molecule comprises a heavy chain variable domain comprising the amino acid sequence of SEQ ID NO: 172 and a light chain variable domain comprising the amino acid sequence of SEQ ID NO: 204.

In some embodiments, the PD-1 inhibitor is chosen from Nivolumab, Pembrolizumab, Pidilizumab, AMP 514, AMP-224, or an anti-PD1 antibody described in U.S. Pat. No. 8,609,089, US 2010028330, and/or US 20120114649, each of which is incorporated herein by reference in its entirety.

In one embodiment, the PD-1 inhibitor is pembrolizumab. In one embodiment, the antibody molecule includes:

(i) a heavy chain variable (VH) region comprising the VHCDR1 amino acid sequence of SEQ ID NO: 503; the VHCDR2 amino acid sequence of SEQ ID NO: 504; and the VHCDR3 amino acid sequence of SEQ ID NO: 505; and

(ii) a light chain variable (VL) region comprising the VLCDR1 amino acid sequence of SEQ ID NO: 500; the VLCDR2 amino acid sequence of SEQ ID NO: 501; and rge VLCDR3 amino acid sequence of SEQ ID NO: 502,

or an amino acid sequence at least 85%, 90%, 95% identical or higher.

### CAR-Expressing Cells

Provided herein are cells, e.g., immune effector cells, that express a chimeric antigen receptor (CAR) that targets, e.g., specifically binds to, an antigen (e.g., EGFRvIII), for use in any of the methods or compositions described herein. The CAR that specifically binds to antigen X is also referred to herein as an “X CAR”. For example, the CAR that specifically binds to EGFRvIII also referred to herein as “an EGFRvIII CAR”. The CAR (e.g., EGFRvIII CAR) expressed by the CAR-expressing cell (e.g., EGFRvIII CAR-expressing cell) described herein includes an antigen binding domain (e.g., EGFRvIII binding domain), a transmembrane domain, and an intracellular signaling domain. In one embodiment, the intracellular signaling domain comprises a costimulatory domain and/or a primary signaling domain.

In embodiments, the CAR molecule comprises an antigen binding domain, transmembrane domain, and an intracellular signaling domain (e.g., an intracellular signaling domain comprising a costimulatory domain and/or a primary signaling domain).

In one embodiment, the CAR molecule comprises an antigen binding domain that is capable of binding an antigen described herein, e.g., a tumor antigen, e.g., chosen from one or more of the following: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

In embodiments, the antigen binding domain of the CAR binds to EGFRvIII.

### EGFRvIII Antigen Binding Domain

In one embodiment, the encoded anti-EGFRvIII binding domain comprises one or more (e.g., all three) light chain complementary determining region 1 (LC CDR1), light chain complementary determining region 2 (LC CDR2), and light chain complementary determining region 3 (LC CDR3) of an anti-EGFRvIII binding domain described herein, and one or more (e.g., all three) heavy chain complementary determining region 1 (HC CDR1), heavy chain complementary determining region 2 (HC CDR2), and heavy chain complementary determining region 3 (HC CDR3) of an anti-EGFRvIII binding domain described herein, e.g., a humanized anti-EGFRvIII binding domain comprising one or more, e.g., all three, LC CDRs and one or more, e.g., all three, HC CDRs. In one embodiment, the encoded anti-EGFRvIII binding domain comprises a light chain variable region described herein (e.g., in Table 2 or SEQ ID NO:11) and/or a heavy chain variable region described herein (e.g., in Table 2 or SEQ ID NO:11). In one embodiment, the encoded anti-EGFRvIII binding domain is a scFv comprising a light chain and a heavy chain of an amino acid sequence of Table 2 or SEQ ID NO:11. In an embodiment, the anti-EGFRvIII binding domain (e.g., an scFv) comprises: a light chain variable region comprising an amino acid sequence having at least one, two or three modifications (e.g., substitutions) but not more than 30, 20 or 10 modifications (e.g., substitutions) of an amino acid sequence of a light chain variable region provided in Table 2 or SEQ ID NO:11, or a sequence with 95-99% identity with an amino acid sequence of Table 2 or SEQ ID NO:11; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided in Table 2 or SEQ ID NO:11, or a sequence with 95-99% identity to an amino acid sequence of Table 2 or SEQ ID NO:11. In one embodiment, the anti-EGFRvIII binding domain comprises a sequence selected from a group consisting of SEQ ID NO:38, SEQ ID NO:44, SEQ ID NO:50, SEQ ID NO:56, SEQ ID NO:62, SEQ ID NO:68, SEQ ID NO:74, SEQ ID NO:80, and SEQ ID NO:86, or a sequence with 95-99% identify thereof. In one embodiment, the nucleic acid sequence encoding the anti-EGFRvIII binding domain comprises a sequence of SEQ ID NO:68. In one embodiment, the nucleic acid sequence encoding the anti-EGFRvIII binding domain comprises a sequence selected from a group consisting of SEQ ID NO:39, SEQ ID NO:45, SEQ ID NO:51, SEQ ID NO:57, SEQ ID NO:63, SEQ ID NO:69, SEQ ID NO:75, SEQ ID NO:81, and SEQ ID NO:98, or a sequence with 95-99% identify thereof. In one embodiment, the encoded anti-EGFRvIII binding domain is a scFv, and a light chain variable region comprising an amino acid sequence described herein, e.g., in Table 2 or SEQ ID NO:11, is attached to a heavy chain variable region comprising an amino acid sequence described herein, e.g., in Table 2 or SEQ ID NO:11, via a linker, e.g., a linker described herein. In one embodiment, the encoded anti-EGFRvIII binding domain includes a (Gly₄-Ser)n linker, wherein n is 1, 2, 3, 4, 5, or 6, preferably 4 (SEQ ID NO: 110). The light chain variable region and heavy chain variable region of a scFv can be, e.g., in any of the following orientations: light chain variable region-linker-heavy chain variable region or heavy chain variable region-linker-light chain variable region.

### Additional Domains of a CAR Molecule and Exemplary CAR Molecules

In one embodiment, the CAR, e.g., EGFRvIII CAR, includes a transmembrane domain that comprises a transmembrane domain of a protein, e.g., a protein described herein, e.g., selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 and CD154.

In one embodiment, the isolated nucleic acid molecule further comprises a sequence encoding a costimulatory domain, e.g., a costimulatory domain described herein. In embodiments, the intracellular signaling domain comprises a costimulatory domain. In embodiments, the intracellular signaling domain comprises a primary signaling domain. In embodiments, the intracellular signaling domain comprises a costimulatory domain and a primary signaling domain.

In one embodiment, the costimulatory domain is a functional signaling domain from a protein, e.g., described herein, e.g., selected from the group consisting of a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83

In one embodiment, the encoded anti-EGFRvIII binding domain comprises one or more (e.g., all three) light chain complementary determining region 1 (LC CDR1), light chain complementary determining region 2 (LC CDR2), and light chain complementary determining region 3 (LC CDR3) of an anti-EGFRvIII binding domain described herein, and one or more (e.g., all three) heavy chain complementary determining region 1 (HC CDR1), heavy chain complementary determining region 2 (HC CDR2), and heavy chain complementary determining region 3 (HC CDR3) of an anti-EGFRvIII binding domain described herein, e.g., a humanized anti-EGFRvIII binding domain comprising one or more, e.g., all three, LC CDRs and one or more, e.g., all three, HC CDRs. In one embodiment, the encoded anti-EGFRvIII binding domain comprises a light chain variable region described herein (e.g., in Table 2 or SEQ ID NO:11) and/or a heavy chain variable region described herein (e.g., in Table 2 or SEQ ID NO:11). In one embodiment, the encoded anti-EGFRvIII binding domain is a scFv comprising a light chain and a heavy chain of an amino acid sequence of Table 2 or SEQ ID NO:11. In an embodiment, the anti-EGFRvIII binding domain (e.g., an scFv) comprises: a light chain variable region comprising an amino acid sequence having at least one, two or three modifications (e.g., substitutions) but not more than 30, 20 or 10 modifications (e.g., substitutions) of an amino acid sequence of a light chain variable region provided in Table 2 or SEQ ID NO:11, or a sequence with 95-99% identity with an amino acid sequence of Table 2 or SEQ ID NO:11; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided in Table 2 or SEQ ID NO:11, or a sequence with 95-99% identity to an amino acid sequence of Table 2 or SEQ ID NO:11. In one embodiment, the anti-EGFRvIII binding domain comprises a sequence selected from a group consisting of SEQ ID NO:38, SEQ ID NO:44, SEQ ID NO:50, SEQ ID NO:56, SEQ ID NO:62, SEQ ID NO:68, SEQ ID NO:74, SEQ ID NO:80, and SEQ ID NO:86, or a sequence with 95-99% identify thereof. In one embodiment, the nucleic acid sequence encoding the anti-EGFRvIII binding domain comprises a sequence of SEQ ID NO:68. In one embodiment, the nucleic acid sequence encoding the anti-EGFRvIII binding domain comprises a sequence selected from a group consisting of SEQ ID NO:39, SEQ ID NO:45, SEQ ID NO:51, SEQ ID NO:57, SEQ ID NO:63, SEQ ID NO:69, SEQ ID NO:75, SEQ ID NO:81, and SEQ ID NO:98, or a sequence with 95-99% identify thereof. In one embodiment, the encoded anti-EGFRvIII binding domain is a scFv, and a light chain variable region comprising an amino acid sequence described herein, e.g., in Table 2 or SEQ ID NO:11, is attached to a heavy chain variable region comprising an amino acid sequence described herein, e.g., in Table 2 or SEQ ID NO:11, via a linker, e.g., a linker described herein. In one embodiment, the encoded anti-EGFRvIII binding domain includes a (Gly₄-Ser)n linker, wherein n is 1, 2, 3, 4, 5, or 6, preferably 4 (SEQ ID NO: 110). The light chain variable region and heavy chain variable region of a scFv can be, e.g., in any of the following orientations: light chain variable region-linker-heavy chain variable region or heavy chain variable region-linker-light chain variable region.

In another aspect, the invention pertains to an isolated polypeptide molecule encoded by the nucleic acid molecule. In one embodiment, the isolated polypeptide molecule comprises a sequence selected from the group consisting of SEQ ID NO:43, SEQ ID NO:49, SEQ ID NO:55, SEQ ID NO:61, SEQ ID NO:67, SEQ ID NO:73, SEQ ID NO:79, SEQ ID NO:85 and SEQ ID NO:90, or a sequence with 95-99% identify thereof. In one embodiment, the isolated polypeptide comprises a sequence of SEQ ID NO:73, or a sequence with 95-99% identify thereof. In one embodiment, the isolated polypeptide comprises a sequence of SEQ ID NO:79, or a sequence with 95-99% identify thereof.

In another aspect, the invention pertains to an isolated chimeric antigen receptor (CAR) molecule comprising an anti-EGFRvIII binding domain (e.g., a humanized antibody or antibody fragment that specifically binds to EGFRvIII), a transmembrane domain, and an intracellular signaling domain (e.g., an intracellular signaling domain comprising a costimulatory domain and/or a primary signaling domain). In one embodiment, the CAR comprises an antibody or antibody fragment which includes an anti-EGFRvIII binding domain described herein (e.g., a humanized antibody or antibody fragment that specifically binds to EGFRvIII as described herein), a transmembrane domain described herein, and an intracellular signaling domain described herein (e.g., an intracellular signaling domain comprising a costimulatory domain and/or a primary signaling domain described herein).

In one embodiment, the anti-EGFRvIII binding domain comprises one or more (e.g., all three) light chain complementary determining region 1 (LC CDR1), light chain complementary determining region 2 (LC CDR2), and light chain complementary determining region 3 (LC CDR3) of an anti-EGFRvIII binding domain described herein, and one or more (e.g., all three) heavy chain complementary determining region 1 (HC CDR1), heavy chain complementary determining region 2 (HC CDR2), and heavy chain complementary determining region 3 (HC CDR3) of an anti-EGFRvIII binding domain described herein, e.g., a humanized anti-EGFRvIII binding domain comprising one or more, e.g., all three, LC CDRs and one or more, e.g., all three, HC CDRs. In one embodiment, the anti-EGFRvIII binding domain comprises a light chain variable region described herein (e.g., in Table 2 or SEQ ID NO:11) and/or a heavy chain variable region described herein (e.g., in Table 2 or SEQ ID NO:11). In one embodiment, the anti-EGFRvIII binding domain is a scFv comprising a light chain and a heavy chain of an amino acid sequence listed in Table 2 or SEQ ID NO:11. In an embodiment, the anti-EGFRvIII binding domain (e.g., an scFv) comprises: a light chain variable region comprising an amino acid sequence having at least one, two or three modifications (e.g., substitutions) but not more than 30, 20 or 10 modifications (e.g., substitutions) of an amino acid sequence of a light chain variable region provided in Table 2 or SEQ ID NO:11, or a sequence with 95-99% identity with an amino acid sequence provided in Table 2 or SEQ ID NO:11; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided in Table 2 or SEQ ID NO:11, or a sequence with 95-99% identity to an amino acid sequence provided in Table 2 or SEQ ID NO:11. In one embodiment, the anti-EGFRvIII binding domain comprises a sequence selected from a group consisting of SEQ ID NO:38, SEQ ID NO:44, SEQ ID NO:50, SEQ ID NO:56, SEQ ID NO:62, SEQ ID NO:68, SEQ ID NO:74, SEQ ID NO:80, and SEQ ID NO:86, or a sequence with 95-99% identify thereof. In one embodiment, the anti-EGFRvIII binding domain is a scFv, and a light chain variable region comprising an amino acid sequence described herein, e.g., in Table 2 or SEQ ID NO:11, is attached to a heavy chain variable region comprising an amino acid sequence described herein, e.g., in Table 2 or SEQ ID NO:11, via a linker, e.g., a linker described herein. In one embodiment, the anti-EGFRvIII binding domain includes a (Gly₄-Ser)n linker, wherein n is 1, 2, 3, 4, 5, or 6, preferably 4 (SEQ ID NO: 110). The light chain variable region and heavy chain variable region of a scFv can be, e.g., in any of the following orientations: light chain variable region-linker-heavy chain variable region or heavy chain variable region-linker-light chain variable region.

In one embodiment, the isolated CAR molecule comprises a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 and CD154. In one embodiment, the transmembrane domain comprises a sequence of SEQ ID NO:15. In one embodiment, the transmembrane domain comprises an amino acid sequence having at least one, two or three modifications (e.g., substitutions) but not more than 20, 10 or 5 modifications (e.g., substitutions) of an amino acid sequence of SEQ ID NO:15, or a sequence with 95-99% identity to an amino acid sequence of SEQ ID NO:15.

In one embodiment, the anti-EGFRvIII binding domain is connected to the transmembrane domain by a hinge region, e.g., a hinge region described herein. In one embodiment, the encoded hinge region comprises SEQ ID NO:14 or SEQ ID NO:104 or SEQ ID NO:106 or SEQ ID NO:108, or a sequence with 95-99% identity thereof.

In one embodiment, the isolated CAR molecule further comprises a sequence encoding a costimulatory domain, e.g., a costimulatory domain described herein. In one embodiment, the costimulatory domain comprises a functional signaling domain of a protein selected from the group consisting of OX40, CD2, CD27, CD28, CDS, ICAM-1, LFA-1 (CD11a/CD18) and 4-1BB (CD137). In one embodiment, the costimulatory domain comprises a sequence of SEQ ID NO:16 or SEQ ID NO:102. In one embodiment, the costimulatory domain comprises an amino acid sequence having at least one, two or three modifications (e.g., substitutions) but not more than 20, 10 or 5 modifications (e.g., substitutions) of an amino acid sequence of SEQ ID NO:16 or SEQ ID NO:102, or a sequence with 95-99% identity to an amino acid sequence of SEQ ID NO:16 or SEQ ID NO:102. In one embodiment, the isolated CAR molecule further comprises a sequence encoding an intracellular signaling domain, e.g., an intracellular signaling domain described herein. In one embodiment, the intracellular signaling domain comprises a functional signaling domain of 4-1BB or CD27 and/or a functional signaling domain of CD3 zeta. In one embodiment, the intracellular signaling domain comprises the sequence of SEQ ID NO: 16 or SEQ ID NO:102 and/or the sequence of SEQ ID NO:17. In one embodiment, the intracellular signaling domain comprises the sequence of SEQ ID NO: 16 or SEQ ID NO:102 and/or the sequence of SEQ ID NO:99. In one embodiment, the intracellular signaling domain comprises an amino acid sequence having at least one, two or three modifications (e.g., substitutions) but not more than 20, 10 or 5 modifications (e.g., substitutions) of an amino acid sequence of SEQ ID NO:16 or SEQ ID NO:102 and/or an amino acid sequence of SEQ ID NO:17 or SEQ ID NO:99, or a sequence with 95-99% identity to an amino acid sequence of SEQ ID NO:16 or SEQ ID NO:102 and/or an amino acid sequence of SEQ ID NO:17 or SEQ ID NO:99. In one embodiment, the intracellular signaling domain comprises the sequence of SEQ ID NO: 16 or SEQ ID NO:102 and the sequence of SEQ ID NO: 17 or SEQ ID NO:99, wherein the sequences comprising the intracellular signaling domain are expressed in the same frame and as a single polypeptide chain.

In one embodiment, the isolated CAR molecule further comprises a leader sequence, e.g., a leader sequence described herein. In one embodiment, the leader sequence comprises an amino acid sequence of SEQ ID NO: 13, or a sequence with 95-99% identity to an amino acid sequence of SEQ ID NO:13.

In another aspect, the invention pertains to an isolated CAR molecule comprising a leader sequence, e.g., a leader sequence described herein, e.g., a leader sequence of SEQ ID NO: 13, or having 95-99% identity thereof, an anti-EGFRvIII binding domain described herein, e.g., an anti-EGFRvIII binding domain comprising a LC CDR1, a LC CDR2, a LC CDR3, a HC CDR1, a HC CDR2 and a HC CDR3 described herein, e.g., an anti-EGFRvIII binding domain described in Table 2 or SEQ ID NO:11, or a sequence with 95-99% identify thereof, a hinge region, e.g., a hinge region described herein, e.g., a hinge region of SEQ ID NO:14 or SEQ ID NO:104 or SEQ ID NO:106 or SEQ ID NO:108, or having 95-99% identity thereof, a transmembrane domain, e.g., a transmembrane domain described herein, e.g., a transmembrane domain having a sequence of SEQ ID NO: 15 or a sequence having 95-99% identity thereof, an intracellular signaling domain, e.g., an intracellular signaling domain described herein (e.g., an intracellular signaling domain comprising a costimulatory domain and/or a primary signaling domain). In one embodiment, the intracellular signaling domain comprises a costimulatory domain, e.g., a costimulatory domain described herein, e.g., a 4-1BB costimulatory domain having a sequence of SEQ ID NO:16 or a CD27 costimulatory domain having a sequence of SEQ ID NO:102, or having 95-99% identity thereof, and/or a primary signaling domain, e.g., a primary signaling domain described herein, e.g., a CD3 zeta stimulatory domain having a sequence of SEQ ID NO:17 or SEQ ID NO:99, or having 95-99% identity thereof. In one embodiment, the intracellular signaling domain comprises a costimulatory domain, e.g., a costimulatory domain described herein, e.g., a 4-1BB costimulatory domain having a sequence of SEQ ID NO:16 or a CD27 costimulatory domain having a sequence of SEQ ID NO:102, and/a primary signaling domain, e.g., a primary signaling domain described herein, e.g., a CD3 zeta stimulatory domain having a sequence of SEQ ID NO:17 or SEQ ID NO:99.

In one embodiment, the isolated CAR molecule comprises (e.g., consists of) an amino acid sequence of SEQ ID NO:43, SEQ ID NO:49, SEQ ID NO:55, SEQ ID NO:61, SEQ ID NO:67, SEQ ID NO:73, SEQ ID NO:79, SEQ ID NO:85, or SEQ ID NO:90, or an amino acid sequence having at least one, two, three, four, five, 10, 15, 20 or 30 modifications (e.g., substitutions) but not more than 60, 50 or 40 modifications (e.g., substitutions) of an amino acid sequence of SEQ ID NO:43, SEQ ID NO:49, SEQ ID NO:55, SEQ ID NO:61, SEQ ID NO:67, SEQ ID NO:73, SEQ ID NO:79, SEQ ID NO:85, or SEQ ID NO:90, or an amino acid sequence having 85%, 90%, 95%, 96%, 97%, 98% or 99% identity to an amino acid sequence of SEQ ID NO:43, SEQ ID NO:49, SEQ ID NO:55, SEQ ID NO:61, SEQ ID NO:67, SEQ ID NO:73, SEQ ID NO:79, SEQ ID NO:85, or SEQ ID NO:90. In one embodiment, the isolated CAR molecule comprises (e.g., consists of) an amino acid sequence of SEQ ID NO:1, or SEQ ID NO:2, or an amino acid sequence having at least one, two, three, four, five, 10, 15, 20 or 30 modifications (e.g., substitutions) but not more than 60, 50 or 40 modifications (e.g., substitutions) of an amino acid sequence of SEQ ID NO:1 or SEQ ID NO:2, or an amino acid sequence having 85%, 90%, 95%, 96%, 97%, 98% or 99% identity to an amino acid sequence of SEQ ID NO:1 or SEQ ID NO:2. In one embodiment, the isolated CAR molecule comprises (e.g., consists of) an amino acid sequence of SEQ ID NO:73, or an amino acid sequence having at least one, two, three, four, five, 10, 15, 20 or 30 modifications (e.g., substitutions) but not more than 60, 50 or 40 modifications (e.g., substitutions) of an amino acid sequence of SEQ ID NO:73, or an amino acid sequence having 85%, 90%, 95%, 96%, 97%, 98% or 99% identity to an amino acid sequence of SEQ ID NO:73. In one embodiment, the isolated CAR molecule comprises (e.g., consists of) an amino acid sequence of SEQ ID NO:79, or an amino acid sequence having at least one, two, three, four, five, 10, 15, 20 or 30 modifications (e.g., substitutions) but not more than 60, 50 or 40 modifications (e.g., substitutions) of an amino acid sequence of SEQ ID NO:79, or an amino acid sequence having 85%, 90%, 95%, 96%, 97%, 98% or 99% identity to an amino acid sequence of SEQ ID NO:79.

In one aspect, the invention pertains to an anti-EGFRvIII binding domain comprising one or more (e.g., all three) light chain complementary determining region 1 (LC CDR1), light chain complementary determining region 2 (LC CDR2), and light chain complementary determining region 3 (LC CDR3) of an anti-EGFRvIII binding domain described herein, and one or more (e.g., all three) heavy chain complementary determining region 1 (HC CDR1), heavy chain complementary determining region 2 (HC CDR2), and heavy chain complementary determining region 3 (HC CDR3) of an anti-EGFRvIII binding domain described herein, e.g., a humanized anti-EGFRvIII binding domain comprising one or more, e.g., all three, LC CDRs and one or more, e.g., all three, HC CDRs. In one embodiment, the anti-EGFRvIII binding domain comprises a light chain variable region described herein (e.g., in SEQ ID NO:38, 44, 50, 56, 62, 68, 74 or 80) and/or a heavy chain variable region described herein (e.g. in SEQ ID NO:38, 44, 50, 56, 62, 68, 74 or 80). In one embodiment, the anti-EGFRvIII binding domain is a scFv comprising a light chain and a heavy chain of an amino acid sequence of in SEQ ID NO:38, 44, 50, 56, 62, 68, 74 or 80. In an embodiment, the anti-EGFRvIII binding domain (e.g., an scFv) comprises: a light chain variable region comprising an amino acid sequence having at least one, two or three modifications (e.g., substitutions) but not more than 30, 20 or 10 modifications (e.g., substitutions) of an amino acid sequence of a light chain variable region provided, in SEQ ID NO:38, 44, 50, 56, 62, 68, 74 or 80 or a sequence with 95-99% identity with an amino acid sequence in SEQ ID NO:38, 44, 50, 56, 62, 68, 74 or 80; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided in SEQ ID NO:38, 44, 50, 56, 62, 68, 74 or 80, or a sequence with 95-99% identity to an amino acid sequence in SEQ ID NO:38, 44, 50, 56, 62, 68, 74 or 80. In one embodiment, the anti-EGFRvIII binding domain comprises a sequence selected from a group consisting of SEQ ID NO:38, SEQ ID NO:44, SEQ ID NO:50, SEQ ID NO:56, SEQ ID NO:62, SEQ ID NO:68, SEQ ID NO:74, SEQ ID NO:80, and SEQ ID NO:86, or a sequence with 95-99% identify thereof. In one embodiment, the anti-EGFRvIII binding domain is a scFv, and a light chain variable region comprising an amino acid sequence described herein, e.g., in Table 2, is attached to a heavy chain variable region comprising an amino acid sequence described herein, e.g., in Table 2, via a linker, e.g., a linker described herein. In one embodiment, the anti-EGFRvIII binding domain includes a (Gly₄-Ser)n linker, wherein n is 1, 2, 3, 4, 5, or 6, preferably 4 (SEQ ID NO: 110). The light chain variable region and heavy chain variable region of a scFv can be, e.g., in any of the following orientations: light chain variable region-linker-heavy chain variable region or heavy chain variable region-linker-light chain variable region.

In another aspect, the invention pertains to a vector comprising a nucleic acid molecule described herein, e.g., a nucleic acid molecule encoding a CAR described herein. In one embodiment, the vector is selected from the group consisting of a DNA, a RNA, a plasmid, a lentivirus vector, adenoviral vector, or a retrovirus vector.

In one embodiment, the vector is a lentivirus vector. In one embodiment, the vector further comprises a promoter. In one embodiment, the promoter is an EF-1 promoter. In one embodiment, the EF-1 promoter comprises a sequence of SEQ ID NO: 97.

In one embodiment, the vector is an in vitro transcribed vector, e.g., a vector that transcribes RNA of a nucleic acid molecule described herein. In one embodiment, the nucleic acid sequence in the vector further comprises a poly(A) tail, e.g., a poly A tail described herein, e.g., comprising about 150 adenosine bases (SEQ ID NO: 111). In one embodiment, the nucleic acid sequence in the vector further comprises a 3′UTR, e.g., a 3′ UTR described herein, e.g., comprising at least one repeat of a 3′UTR derived from human beta-globulin.

In another aspect, the invention pertains to a cell comprising a vector described herein. In one embodiment, the cell is a cell described herein, e.g., a human T cell, e.g., a human T cell described herein. In one embodiment, the human T cell is a CD8+ T cell.

In another aspect, the invention pertains to a method of making a cell comprising transducing a cell described herein, e.g., a T cell described herein, with a vector of comprising a nucleic acid encoding a CAR, e.g., a CAR described herein.

The present invention also provides a method of generating a population of RNA-engineered cells, e.g., cells described herein, e.g., T cells, transiently expressing exogenous RNA. The method comprises introducing an in vitro transcribed RNA or synthetic RNA into a cell, where the RNA comprises a nucleic acid encoding a CAR molecule described herein.

In an embodiment, the CAR molecule comprises a CD123 CAR described herein, e.g., a CD123 CAR described in US2014/0322212A1 or US2016/0068601A1, both incorporated herein by reference. In embodiments, the CD123 CAR comprises an amino acid, or has a nucleotide sequence shown in US2014/0322212A1 or US2016/0068601A1, both incorporated herein by reference.

In embodiments, the CAR molecule comprises a CD19 CAR molecule described herein, e.g., a CD19 CAR molecule described in US-2015-0283178-A1, e.g., CTL019. In embodiments, the CD19 CAR comprises an amino acid, or has a nucleotide sequence shown in US-2015-0283178-A1, incorporated herein by reference.

In one embodiment, CAR molecule comprises a BCMA CAR molecule described herein, e.g., a BCMA CAR described in US-2016-0046724-A1. In embodiments, the BCMA CAR comprises an amino acid, or has a nucleotide sequence shown in US-2016-0046724-A1, incorporated herein by reference.

In an embodiment, the CAR molecule comprises a CLL1 CAR described herein, e.g., a CLL1 CAR described in US2016/0051651A1, incorporated herein by reference. In embodiments, the CLL1 CAR comprises an amino acid, or has a nucleotide sequence shown in US2016/0051651A1, incorporated herein by reference.

In an embodiment, the CAR molecule comprises a CD33 CAR described herein, e.g. a CD33 CAR described in US2016/0096892A1, incorporated herein by reference. In embodiments, the CD33 CAR comprises an amino acid, or has a nucleotide sequence shown in US2016/0096892A1, incorporated herein by reference.

In an embodiment, the CAR molecule comprises an EGFRvIII CAR molecule described herein, e.g., an EGFRvIII CAR described US2014/0322275A1, incorporated herein by reference.

In embodiments, the EGFRvIII CAR comprises an amino acid, or has a nucleotide sequence shown in US2014/0322275A1, incorporated herein by reference.

In an embodiment, the CAR molecule comprises a mesothelin CAR described herein, e.g., a mesothelin CAR described in WO 2015/090230, incorporated herein by reference. In embodiments, the mesothelin CAR comprises an amino acid, or has a nucleotide sequence shown in WO 2015/090230, incorporated herein by reference.

In embodiments of any of the methods and compositions described herein, the cell comprising a CAR comprises a nucleic acid encoding the CAR.

In one embodiment, the nucleic acid encoding the CAR is a lentiviral vector. In one embodiment, the nucleic acid encoding the CAR is introduced into the cells by lentiviral transduction. In one embodiment, the nucleic acid encoding the CAR is an RNA, e.g., an in vitro transcribed RNA. In one embodiment, the nucleic acid encoding the CAR is introduced into the cells by electroporation.

In embodiments of any of the methods and compositions described herein, the cell is a T cell or an NK cell. In one embodiment, the T cell is an autologous or allogeneic T cell.

### Subjects

In one embodiment, the subject, e.g., the subject from which immune cells are acquired and/or the subject to be treated, is a human, e.g., a cancer patient. In certain embodiments, the subject is 18 years of age or younger (e.g., 18, 17, 16, 15, 14, 13, 12, 11, 10, 9, 8, 7, 6, 5, 4, 3, 2, 1 year or younger (e.g., 12 months, 6 months, 3 months or less)). In one embodiment, the subject is a pediatric cancer patient.

In other embodiments, the subject is an adult, e.g., the subject is older than 18 years of age (e.g., older than 18, 19, 20, 25, 30, 35, 40, 45, 50, 55, 60, 65, 70, 75, 80, or older). In one embodiment, the subject is an adult cancer patient.

In certain embodiments, the subject has a disease associated with expression of a tumor- or cancer associated-antigen, e.g., a disease as described herein. In one embodiment, the subject has a cancer, e.g., a cancer as described herein.

In one embodiment, the subject has a cancer that is chosen from a hematological cancer, a solid tumor, or a metastatic lesion thereof. Exemplary cancers include, but are not limited to,

B-cell acute lymphocytic leukemia (B-ALL), T-cell acute lymphocytic leukemia (T-ALL), acute gliobastoma (GBM), e.g. glioblastoma (GBM) (e.g., IDH-wildtype GBM, IDH-mutant GBM, or MGMT-unmethylated GBM). In one embodiment, the cancer is IDH-wildtype GBM. In another embodiment, the cancer is IDH-mutant GBM. In one embodiment, the cancer is MGMT-unmethylated GBM.

In embodiments, the subject has a GBM, wherein MGMT is unmethylated. In embodiments, the subject has MGMT-unmethlated GBM, and is a adult patient, e.g., is older than 18 years of age (e.g., older than 18, 19, 20, 25, 30, 35, 40, 45, 50, 55, 60, 65, 70, 75, 80, or older).

In embodiments, the subject has (e.g., is diagnosed with) a disease (e.g., cancer) described herein, e.g., a disease associated with EGFRvIII expression, e.g., a cancer associated with EGFRvIII expression described herein. In embodiments, the subject has a relapsed and/or refractory cancer, e.g., relapsed or refractory GBM, e.g., EGFRvIII+ GBM. In embodiments, the subject has a cancer in stage I, II, III, or IV. In embodiments, the subject has a tumor mass of at least 1, 2, 5, 10, 20, 50, 100, 200, 500, or 1000 g, e.g., in a single tumor or a plurality of tumors.

In embodiments, the subject has been administered a chemotherapy, e.g., a chemotherapy described herein (e.g., lymphodepleting chemotherapy, e.g., carboplatin and/or gemcitabine), prior to administration with a CAR-expressing cell and/or a PD-1 inhibitor described herein. In embodiments, the subject has been administered an immunotherapy, e.g., an allogeneic bone marrow transplant, prior to administration with a CAR-expressing cell and/or a PD-1 inhibitor described herein.

In embodiments of any of the methods and compositions described herein, the subject is a mammal, e.g., a human. In one embodiment, the subject expresses PD-1. In one embodiment, the cancer cell or a cell in close proximity to a cancer cell, e.g., a cancer-associated cell, in the subject expresses PD-1 or PL-L1. In an embodiment, the cancer-associated cell is an anti-tumor immune cell, e.g., a tumor infiltrating lymphocyte (TIL).

In one embodiment, the cell expressing a CAR, e.g., an EGFRvIII CAR-expressing cell described herein, expresses PD-1.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting. Headings, sub-headings or numbered or lettered elements, e.g., (a), (b), (i) etc., are presented merely for ease of reading. The use of headings or numbered or lettered elements in this document does not require the steps or elements be performed in alphabetical order or that the steps or elements are necessarily discrete from one another. Other features, objects, and advantages of the invention will be apparent from the description and drawings, and from the claims.

### BRIEF DESCRIPTION OF THE DRAWINGS

FIG. 1 demonstrates that immune checkpoint blockade with PD-1 enhances EGFRvIII CAR T cell function in NSG mice with a D270 GBM subcutaneous model.

FIG. 2 provides an exemplary schematic for a phase 1 study of the combination therapy of a CAR-expressing cell (e.g., EGFRvIII CAR-expressing cell) described herein and a PD-1 inhibitor described herein.

### DETAILED DESCRIPTION

### Definitions

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

The term “Chimeric Antigen Receptor” or alternatively a “CAR” refers to a recombinant polypeptide construct comprising at least an extracellular antigen binding domain, a transmembrane domain and a cytoplasmic signaling domain (also referred to herein as “an intracellular signaling domain”) comprising a functional signaling domain derived from a stimulatory molecule as defined below. In some embodiments, the domains in the CAR polypeptide construct are in the same polypeptide chain, e.g., comprise a chimeric fusion protein. In some embodiments, the domains in the CAR polypeptide construct are not contiguous with each other, e.g., are in different polypeptide chains, e.g., as provided in an RCAR as described herein.

In one aspect, the stimulatory molecule of the CAR is the zeta chain associated with the T cell receptor complex. In one aspect, the cytoplasmic signaling domain comprises a primary signaling domain (e.g., a primary signaling domain of CD3-zeta). In one aspect, the cytoplasmic signaling domain further comprises one or more functional signaling domains derived from at least one costimulatory molecule as defined below. In one aspect, the costimulatory molecule is chosen from 4 1BB (i.e., CD137), CD27, ICOS, and/or CD28. In one aspect, the CAR comprises a chimeric fusion protein comprising an extracellular antigen recognition domain, a transmembrane domain and an intracellular signaling domain comprising a functional signaling domain derived from a stimulatory molecule. In one aspect, the CAR comprises a chimeric fusion protein comprising an extracellular antigen recognition domain, a transmembrane domain and an intracellular signaling domain comprising a functional signaling domain derived from a co-stimulatory molecule and a functional signaling domain derived from a stimulatory molecule. In one aspect, the CAR comprises a chimeric fusion protein comprising an extracellular antigen recognition domain, a transmembrane domain and an intracellular signaling domain comprising two functional signaling domains derived from one or more co-stimulatory molecule(s) and a functional signaling domain derived from a stimulatory molecule. In one aspect, the CAR comprises a chimeric fusion protein comprising an extracellular antigen recognition domain, a transmembrane domain and an intracellular signaling domain comprising at least two functional signaling domains derived from one or more co-stimulatory molecule(s) and a functional signaling domain derived from a stimulatory molecule. In one aspect the CAR comprises an optional leader sequence at the amino-terminus (N-ter) of the CAR fusion protein. In one aspect, the CAR further comprises a leader sequence at the N-terminus of the extracellular antigen recognition domain, wherein the leader sequence is optionally cleaved from the antigen recognition domain (e.g., an scFv) during cellular processing and localization of the CAR to the cellular membrane.

A CAR that comprises an antigen binding domain (e.g., an scFv, a single domain antibody, or TCR (e.g., a TCR alpha binding domain or TCR beta binding domain)) that targets a specific tumor marker X, wherein X can be a tumor marker as described herein, is also referred to as XCAR. For example, a CAR that comprises an antigen binding domain that targets EGFRvIII is referred to as an EGFRvIII CAR. The CAR can be expressed in any cell, e.g., an immune effector cell as described herein (e.g., a T cell or an NK cell).

The term “signaling domain” refers to the functional portion of a protein which acts by transmitting information within the cell to regulate cellular activity via defined signaling pathways by generating second messengers or functioning as effectors by responding to such messengers. In some aspects, the signaling domain of the CAR described herein is derived from a stimulatory molecule or co-stimulatory molecule described herein, or is a synthesized or engineered signaling domain.

The term “EGFR” refers to any mammalian mature full-length epidermal growth factor receptor, including human and non-human forms. The 1186 amino acid human EGFR is described in Ullrich et al., Nature 309:418-425 (1984)) and GenBank Accession No. AF125253 and SwissProt Acc No P00533-2.

The term “EGFRvIII” refers to Epidermal growth factor receptor variant III. EGFRvIII is the most common variant of EGFR observed in human tumors but is rarely observed in normal tissue. This protein results from the in-frame deletion of exons 2-7 and the generation of a novel glycine residue at the junction of exons 1 and 8 within the extra-cellular domain of the EGFR, thereby creating a tumor specific epitope. EGFRvIII is expressed in 24% to 67% of GBM, but not in normal tissues. EGFRvIII is also known as type III mutant, delta-EGFR, EGFRde2-7, and □EGFR and is described in U.S. Pat. Nos. 6,455,498, 6,127,126, 5,981,725, 5,814,317, 5,710,010, 5,401,828, and 5,212,290. Expression of EGFRvIII may result from a chromosomal deletion, and may also result from aberrant alternative splicing. See Sugawa et al., 1990, Proc. Natl. Acad. Sci. 87:8602-8606.

The term “antibody,” as used herein, refers to a protein, or polypeptide sequence derived from an immunoglobulin molecule, which specifically binds with an antigen. Antibodies can be polyclonal or monoclonal, multiple or single chain, or intact immunoglobulins, and may be derived from natural sources or from recombinant sources. Antibodies can be tetramers of immunoglobulin molecules.

The term “antibody fragment” refers to at least one portion of an intact antibody, or recombinant variants thereof, and refers to the antigen binding domain, e.g., an antigenic determining variable region of an intact antibody, that is sufficient to confer recognition and specific binding of the antibody fragment to a target, such as an antigen. Examples of antibody fragments include, but are not limited to, Fab, Fab′, F(ab′)2, and Fv fragments, scFv antibody fragments, linear antibodies, single domain antibodies such as sdAb (either VL or VH), camelid VHH domains, and multi-specific molecules formed from antibody fragments such as a bivalent fragment comprising two or more, e.g., two, Fab fragments linked by a disulfide brudge at the hinge region, or two or more, e.g., two isolated CDR or other epitope binding fragments of an antibody linked. An antibody fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antibody fragments can also be grafted into scaffolds based on polypeptides such as a fibronectin type III (Fn3) (see U.S. Pat. No. 6,703,199, which describes fibronectin polypeptide minibodies).

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The terms “complementarity determining region” or “CDR,” as used herein, refer to the sequences of amino acids within antibody variable regions which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273,927-948 (“Chothia” numbering scheme), or a combination thereof. Under the Kabat numbering scheme, in some embodiments, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under the Chothia numbering scheme, in some embodiments, the CDR amino acids in the VH are numbered 26-32 (HCDR1), 52-56 (HCDR2), and 95-102 (HCDR3); and the CDR amino acid residues in the VL are numbered 26-32 (LCDR1), 50-52 (LCDR2), and 91-96 (LCDR3). In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35 (HCDR1), 50-65 (HCDR2), and 95-102 (HCDR3) in a VH, e.g., a mammalian VH, e.g., a human VH; and amino acid residues 24-34 (LCDR1), 50-56 (LCDR2), and 89-97 (LCDR3) in a VL, e.g., a mammalian VL, e.g., a human VL.

The portion of the CAR composition of the invention comprising an antibody or antibody fragment thereof may exist in a variety of forms, for example, where the antigen binding domain is expressed as part of a polypeptide chain including, for example, a single domain antibody fragment (sdAb), a single chain antibody (scFv), or e.g., a humanized antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In one aspect, the antigen binding domain of a CAR composition of the invention comprises an antibody fragment. In a further aspect, the CAR comprises an antibody fragment that comprises an scFv.

As used herein, the term “binding domain” or “antibody molecule refers to a protein, e.g., an immunoglobulin chain or fragment thereof, comprising at least one immunoglobulin variable domain sequence. The term “binding domain” or “antibody molecule” encompasses antibodies and antibody fragments. In an embodiment, an antibody molecule is a multispecific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (κ) and lambda (λ) light chains refer to the two major antibody light chain isotypes.

The term “recombinant antibody” refers to an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “antigen” or “Ag” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components.

The term “anti-tumor effect” refers to a biological effect which can be manifested by various means, including but not limited to, e.g., a decrease in tumor volume, a decrease in the number of tumor cells, a decrease in the number of metastases, an increase in life expectancy, decrease in tumor cell proliferation, decrease in tumor cell survival, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

The term “anti-cancer effect” refers to a biological effect which can be manifested by various means, including but not limited to, e.g., a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anti-cancer effect” can also be manifested by the ability of the peptides, polynucleotides, cells and antibodies in prevention of the occurrence of cancer in the first place. The term “anti-tumor effect” refers to a biological effect which can be manifested by various means, including but not limited to, e.g., a decrease in tumor volume, a decrease in the number of tumor cells, a decrease in tumor cell proliferation, or a decrease in tumor cell survival. The term “autologous” refers to any material derived from the same individual to whom it is later to be re-introduced into the individual.

The term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term “xenogeneic” refers to a graft derived from an animal of a different species.

The term “apheresis” as used herein refers to the art-recognized extracorporeal process by which the blood of a donor or patient is removed from the donor or patient and passed through an apparatus that separates out selected particular constituent(s) and returns the remainder to the circulation of the donor or patient, e.g., by retransfusion. Thus, in the context of “an apheresis sample” refers to a sample obtained using apheresis.

The term “combination” refers to either a fixed combination in one dosage unit form, or a combined administration where a compound of the present invention and a combination partner (e.g. another drug as explained below, also referred to as “therapeutic agent” or “co-agent”) may be administered independently at the same time or separately within time intervals, especially where these time intervals allow that the combination partners show a cooperative, e.g. synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g. a compound of the present invention and a combination partner, are both administered to a patient simultaneously in the form of a single entity or dosage. The term “non-fixed combination” means that the active ingredients, e.g. a compound of the present invention and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active ingredients.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to, glioblastoma, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

The terms “cancer associated antigen” or “tumor antigen” or “proliferative disorder antigen” or “antigen associated with a proliferative disorder” interchangeably refers to a molecule (typically protein, carbohydrate or lipid) that is preferentially expressed on the surface of a cancer cell, either entirely or as a fragment (e.g., MHC/peptide), in comparison to a normal cell, and which is useful for the preferential targeting of a pharmacological agent to the cancer cell. The term “disease associated with expression of EGFRvIII” as used herein includes, but is not limited to, a disease associated with expression of EGFRvIII or condition associated with cells which express EGFRvIII including, tumor cells of various cancers such as, e.g., glioblastoma (including glioblastoma stem cells); breast, ovarian, and non-small cell lung carcinomas; head and neck squamous cell carcinoma; medulloblastoma, colorectal cancer, prostate cancer, and bladder carcinoma. Without being bound to a particular theory or mechanism, it is believed that by eliciting an antigen-specific response against EGFRvIII, the CARs disclosed herein provide for one or more of the following: targeting and destroying EGFRvIII-expressing tumor cells, reducing or eliminating tumors, facilitating infiltration of immune cells to the tumor site, and enhancing/extending anti-tumor responses. Because EGFRvIII is not expressed at detectable levels in normal (i.e., non-cancerous) tissue, it is contemplated that the inventive CARs advantageously substantially avoid targeting/destroying normal tissues and cells.

As used herein, the term “Programmed Death 1” or “PD-1” include isoforms, mammalian, e.g., human PD-1, species homologs of human PD-1, and analogs comprising at least one common epitope with PD-1. The amino acid sequence of PD-1, e.g., human PD-1, is known in the art, e.g., Shinohara T et al. (1994) Genomics 23(3):704-6; Finger L R, et al. Gene (1997) 197(1-2):177-87.

The term “conservative sequence modifications” is intended to refer to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the invention by standard techniques known in the art, such as site-directed mutagenesis and PCR-mediated mutagenesis. Conservative amino acid.

The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

“Derived from” as that term is used herein, indicates a relationship between a first and a second molecule. It generally refers to structural similarity between the first molecule and a second molecule and does not connotate or include a process or source limitation on a first molecule that is derived from a second molecule. For example, in the case of an intracellular signaling domain that is derived from a CD3zeta molecule, the intracellular signaling domain retains sufficient CD3zeta structure such that is has the required function, namely, the ability to generate a signal under the appropriate conditions. It does not connotate or include a limitation to a particular process of producing the intracellular signaling domain, e.g., it does not mean that, to provide the intracellular signaling domain, one must start with a CD3zeta sequence and delete unwanted sequence, or impose mutations, to arrive at the intracellular signaling domain.

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the invention by standard techniques known in the art, such as site-directed mutagenesis and PCR-mediated mutagenesis. Conservative substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a CAR of the invention can be replaced with other amino acid residues from the same side chain family and the altered CAR can be tested using the functional assays described herein.

The term “stimulation,” refers to a primary response induced by binding of a stimulatory molecule (e.g., a TCR/CD3 complex) with its cognate ligand thereby mediating a signal transduction event, such as, but not limited to, signal transduction via the TCR/CD3 complex.

Stimulation can mediate altered expression of certain molecules, such as downregulation of TGF-β, and/or reorganization of cytoskeletal structures, and the like.

The term “stimulatory molecule,” refers to a molecule expressed by a T cell that provides the primary cytoplasmic signaling sequence(s) that regulate primary activation of the TCR complex in a stimulatory way for at least some aspect of the T cell signaling pathway. In some embodiments, the ITAM-containing domain within the CAR recapitulates the signaling of the primary TCR independently of endogenous TCR complexes. In one aspect, the primary signal is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or ITAM. Examples of an ITAM containing primary cytoplasmic signaling sequence that is of particular use in the invention includes, but is not limited to, those derived from TCR zeta, FcR gamma, FcR beta, CD3 gamma, CD3 delta, CD3 epsilon, CDS, CD22, CD79a, CD79b, CD278 (also known as “ICOS”), FcεRI and CD66d, DAP10 and DAP12. In a specific CAR of the invention, the intracellular signaling domain in any one or more CARs of the invention comprises an intracellular signaling sequence, e.g., a primary signaling sequence of CD3-zeta. In a specific CAR of the invention, the primary signaling sequence of CD3-zeta is the amino acid sequence provided as SEQ ID NO: 17, or the equivalent residues from a non-human species, e.g., mouse, rodent, monkey, ape and the like. In a specific CAR of the invention, the primary signaling sequence of CD3-zeta is the amino acid sequence as provided in SEQ ID NO: 99, or the equivalent residues from a non-human species, e.g., mouse, rodent, monkey, ape and the like.

The term “antigen presenting cell” or “APC” refers to an immune system cell such as an accessory cell (e.g., a B-cell, a dendritic cell, and the like) that displays a foreign antigen complexed with major histocompatibility complexes (MHC's) on its surface. T-cells may recognize these complexes using their T-cell receptors (TCRs). APCs process antigens and present them to T-cells.

An “intracellular signaling domain,” as the term is used herein, refers to an intracellular portion of a molecule. In embodiments, the intracellular signal domain transduces the effector function signal and directs the cell to perform a specialized function. While the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

The intracellular signaling domain generates a signal that promotes an immune effector function of the CAR containing cell, e.g., a CART cell. Examples of immune effector function, e.g., in a CART cell, include cytolytic activity and helper activity, including the secretion of cytokines.

In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation. For example, in the case of a CART, a primary intracellular signaling domain can comprise a cytoplasmic sequence of a T cell receptor, and a costimulatory intracellular signaling domain can comprise cytoplasmic sequence from co-receptor or costimulatory molecule.

A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, FcR gamma, FcR beta, CD3 gamma, CD3 delta, CD3 epsilon, CD5, CD22, CD79a, CD79b, CD278 (also known as “ICOS”), FcεRI, CD66d, DAP10 and DAP12.

The term “zeta” or alternatively “zeta chain”, “CD3-zeta” or “TCR-zeta” is defined as the protein provided as GenBan Acc. No. BAG36664.1, or the equivalent residues from a non-human species, e.g., mouse, rodent, monkey, ape and the like, and a “zeta stimulatory domain” or alternatively a “CD3-zeta stimulatory domain” or a “TCR-zeta stimulatory domain” is defined as the amino acid residues from the cytoplasmic domain of the zeta chain that are sufficient to functionally transmit an initial signal necessary for T cell activation. In one aspect the cytoplasmic domain of zeta comprises residues 52 through 164 of GenBank Acc. No. BAG36664.1 or the equivalent residues from a non-human species, e.g., mouse, rodent, monkey, ape and the like, that are functional orthologs thereof. In one aspect, the “zeta stimulatory domain” or a “CD3-zeta stimulatory domain” is the sequence provided as SEQ ID NO:17. In one aspect, the “zeta stimulatory domain” or a “CD3-zeta stimulatory domain” is the sequence provided as SEQ ID NO:99. Also encompassed herein are CD3 zeta domains comprising one or more mutations to the amino acid sequences described herein, e.g., SEQ ID NO: 99.

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include, but are not limited to an MHC class I molecule, TNF receptor proteins, Immunoglobulin-like proteins, cytokine receptors, integrins, signaling lymphocytic activation molecules (SLAM proteins), activating NK cell receptors, BTLA, Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

A costimulatory intracellular signaling domain refers to the intracellular portion of a costimulatory molecule.

The intracellular signaling domain can comprise the entire intracellular portion, or the entire native intracellular signaling domain, of the molecule from which it is derived, or a functional fragment thereof.

The term “4-1BB” refers to a member of the TNFR superfamily with an amino acid sequence provided as GenBank Acc. No. AAA62478.2, or the equivalent residues from a non-human species, e.g., mouse, rodent, monkey, ape and the like; and a “4-1BB costimulatory domain” is defined as amino acid residues 214-255 of GenBank Acc. No. AAA62478.2, or the equivalent residues from a non-human species, e.g., mouse, rodent, monkey, ape and the like. In one aspect, the “4-1BB costimulatory domain” is the sequence provided as SEQ ID NO:16 or the equivalent residues from a non-human species, e.g., mouse, rodent, monkey, ape and the like

“Immune effector cell,” as that term is used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and myeloic-derived phagocytes.

“Immune effector function or immune effector response,” as that term is used herein, refers to function or response, e.g., of an immune effector cell, that enhances or promotes an immune attack of a target cell. E.g., an immune effector function or response refers a property of a T or NK cell that promotes killing or the inhibition of growth or proliferation, of a target cell. In the case of a T cell, primary stimulation and co-stimulation are examples of immune effector function or response.

The term “effector function” refers to a specialized function of a cell. Effector function of a T cell, for example, may be cytolytic activity or helper activity including the secretion of cytokines.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

The term “effective amount” or “therapeutically effective amount” are used interchangeably herein, and refer to an amount of a compound, formulation, material, or composition, as described herein effective to achieve a particular biological result.

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence driven by a promoter.

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

The term “human” antibody refers to fully human antibodies as well as effectively human antibodies. “Fully human” refers to an immunoglobulin, such as an antibody or antibody fragment, where the whole molecule is of human origin or consists of an amino acid sequence identical to a human form of the antibody or immunoglobulin. An “effectively human” antibody is an antibody that includes a sufficient number of human amino acid positions such that the antibody does not elicit an immunogenic response in a normal human.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

The term “parenteral” administration of an immunogenic composition includes, e.g., subcutaneous (s.c.), intravenous (i.v.), intramuscular (i.m.), or intrasternal injection, intratumoral, or infusion techniques.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions, e.g., conservative substitutions), alleles, orthologs, SNPs, and complementary sequences as well as the sequence explicitly indicated. Specifically, degenerate codon substitutions, e.g., conservative substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “promoter” refers to a DNA sequence recognized by the synthetic machinery of the cell, or introduced synthetic machinery, required to initiate the specific transcription of a polynucleotide sequence.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “constitutive” promoter refers to a nucleotide sequence which, when operably linked with a polynucleotide which encodes or specifies a gene product, causes the gene product to be produced in a cell under most or all physiological conditions of the cell.

The term “inducible” promoter refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific” promoter refers to a nucleotide sequence which, when operably linked with a polynucleotide encodes or specified by a gene, causes the gene product to be produced in a cell substantially only if the cell is a cell of the tissue type corresponding to the promoter.

The terms “cancer associated antigen” or “tumor antigen” interchangeably refers to a molecule (typically a protein, carbohydrate or lipid) that is expressed on the surface of a cancer cell, either entirely or as a fragment (e.g., MHC/peptide), and which is useful for the preferential targeting of a pharmacological agent to the cancer cell. In some embodiments, a tumor antigen is a marker expressed by both normal cells and cancer cells, e.g., a lineage marker. In some embodiments, a tumor antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a tumor antigen is a cell surface molecule that is inappropriately synthesized in the cancer cell, for instance, a molecule that contains deletions, additions or mutations in comparison to the molecule expressed on a normal cell. In some embodiments, a tumor antigen will be expressed exclusively on the cell surface of a cancer cell, entirely or as a fragment (e.g., MHC/peptide), and not synthesized or expressed on the surface of a normal cell. In some embodiments, the CARs of the present invention includes CARs comprising an antigen binding domain (e.g., antibody or antibody fragment) that binds to a MHC presented peptide. Normally, peptides derived from endogenous proteins fill the pockets of Major histocompatibility complex (MHC) class I molecules, and are recognized by T cell receptors (TCRs) on CD8+T lymphocytes. The MHC class I complexes are constitutively expressed by all nucleated cells. In cancer, virus-specific and/or tumor-specific peptide/MHC complexes represent a unique class of cell surface targets for immunotherapy. TCR-like antibodies targeting peptides derived from viral or tumor antigens in the context of human leukocyte antigen (HLA)-A1 or HLA-A2 have been described (see, e.g., Sastry et al., J Virol. 2011 85(5):1935-1942; Sergeeva et al., Blood, 2011 117(16):4262-4272; Verma et al., J Immunol 2010 184(4):2156-2165; Willemsen et al., Gene Ther 2001 8(21):1601-1608; Dao et al., Sci Transl Med 2013 5(176):176ra33; Tassev et al., Cancer Gene Ther 2012 19(2):84-100). For example, TCR-like antibody can be identified from screening a library, such as a human scFv phage displayed library.

The term “tumor-supporting antigen” or “cancer-supporting antigen” interchangeably refer to a molecule (typically a protein, carbohydrate or lipid) that is expressed on the surface of a cell that is, itself, not cancerous, but supports the cancer cells, e.g., by promoting their growth or survival e.g., resistance to immune cells. Exemplary cells of this type include stromal cells and myeloid-derived suppressor cells (MDSCs). The tumor-supporting antigen itself need not play a role in supporting the tumor cells so long as the antigen is present on a cell that supports cancer cells.

The term “flexible polypeptide linker” or “linker” as used in the context of an scFv refers to a peptide linker that consists of amino acids such as glycine and/or serine residues used alone or in combination, to link variable heavy and variable light chain regions together. In one embodiment, the flexible polypeptide linker is a Gly/Ser linker and comprises the amino acid sequence (Gly-Gly-Gly-Ser)n (SEQ ID NO: 112), where n is a positive integer equal to or greater than 1. For example, n=1, n=2, n=3. n=4, n=5 and n=6, n=7, n=8, n=9 and n=10. In one embodiment, the flexible polypeptide linkers include, but are not limited to, (Gly4 Ser)4 (SEQ ID NO: 113) or (Gly4 Ser)3 (SEQ ID NO: 114). In another embodiment, the linkers include multiple repeats of (Gly2Ser), (GlySer) or (Gly3Ser) (SEQ ID NO: 112). Also included within the scope of the invention are linkers described in WO2012/138475, incorporated herein by reference).

As used herein, a 5′ cap (also termed an RNA cap, an RNA 7-methylguanosine cap or an RNA m7G cap) is a modified guanine nucleotide that has been added to the “front” or 5′ end of a eukaryotic messenger RNA shortly after the start of transcription. The 5′ cap consists of a terminal group which is linked to the first transcribed nucleotide. Its presence is critical for recognition by the ribosome and protection from RNases. Cap addition is coupled to transcription, and occurs co-transcriptionally, such that each influences the other. Shortly after the start of transcription, the 5′ end of the mRNA being synthesized is bound by a cap-synthesizing complex associated with RNA polymerase. This enzymatic complex catalyzes the chemical reactions that are required for mRNA capping. Synthesis proceeds as a multi-step biochemical reaction. The capping moiety can be modified to modulate functionality of mRNA such as its stability or efficiency of translation.

As used herein, “in vitro transcribed RNA” refers to RNA, preferably mRNA, that has been synthesized in vitro. Generally, the in vitro transcribed RNA is generated from an in vitro transcription vector. The in vitro transcription vector comprises a template that is used to generate the in vitro transcribed RNA.

As used herein, a “poly(A)” is a series of adenosines attached by polyadenylation to the mRNA. In the preferred embodiment of a construct for transient expression, the polyA is between 50 and 5000 (SEQ ID NO: 115), preferably greater than 64, more preferably greater than 100, most preferably greater than 300 or 400. poly(A) sequences can be modified chemically or enzymatically to modulate mRNA functionality such as localization, stability or efficiency of translation.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

As used herein, the terms “treat”, “treatment” and “treating” refer to the reduction or amelioration of the progression, severity and/or duration of a proliferative disorder, or the amelioration of one or more symptoms (preferably, one or more discernible symptoms) of a proliferative disorder resulting from the administration of one or more therapies (e.g., one or more therapeutic agents such as a CAR of the invention). In specific embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating”-refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

A dosage regimen, e.g., a therapeutic dosage regimen, can include one or more treatment intervals. The dosage regimen can result in at least one beneficial or desired clinical result including, but are not limited to, alleviation of a symptom, diminishment of extent of disease, stabilized (i.e., not worsening) state of disease, delay or slowing of disease progression, amelioration or palliation of the disease state, whether detectable or undetectable.

As used herein, a “treatment interval” refers to a treatment cycle, for example, a course of administration of a therapeutic agent that can be repeated, e.g., on a regular schedule. In embodiments, a dosage regimen can have one or more periods of no administration of the therapeutic agent in between treatment intervals. For example, a treatment interval can include one dose of a CAR molecule administered in combination with (prior, concurrently or after) administration of a second therapeutic agent, e.g., an inhibitor (e.g., a kinase inhibitor as described herein).

The term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (e.g., mammals, human). In an embodiment, a subject is a mammal. In an embodiment, a subject is a human. In an embodiment, a subject is a patient. In one embodiment, the subject is a pediatric subject. In other embodiments, the subject is an adult.

The term, a “substantially purified” cell refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “specifically binds,” refers to an antibody, or a ligand, which recognizes and binds with a cognate binding partner (e.g., a stimulatory and/or costimulatory molecule present on a T cell) protein present in a sample, but which antibody or ligand does not substantially recognize or bind other molecules in the sample.

“Regulatable chimeric antigen receptor (RCAR),” as used herein, refers to a set of polypeptides, typically two in the simplest embodiments, which when in an immune effector cell, provides the cell with specificity for a target cell, typically a cancer cell, and with intracellular signal generation. In some embodiments, an RCAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined herein in the context of a CAR molecule. In some embodiments, the set of polypeptides in the RCAR are not contiguous with each other, e.g., are in different polypeptide chains. In some embodiments, the RCAR includes a dimerization switch that, upon the presence of a dimerization molecule, can couple the polypeptides to one another, e.g., can couple an antigen binding domain to an intracellular signaling domain. In some embodiments, the RCAR is expressed in a cell (e.g., an immune effector cell) as described herein, e.g., an RCAR-expressing cell (also referred to herein as “RCARX cell”). In an embodiment the RCARX cell is a T cell, and is referred to as a RCART cell. In an embodiment the RCARX cell is an NK cell, and is referred to as a RCARN cell. The RCAR can provide the RCAR-expressing cell with specificity for a target cell, typically a cancer cell, and with regulatable intracellular signal generation or proliferation, which can optimize an immune effector property of the RCAR-expressing cell. In embodiments, an RCAR cell relies at least in part, on an antigen binding domain to provide specificity to a target cell that comprises the antigen bound by the antigen binding domain.

“Membrane anchor” or “membrane tethering domain”, as that term is used herein, refers to a polypeptide or moiety, e.g., a myristoyl group, sufficient to anchor an extracellular or intracellular domain to the plasma membrane.

“Switch domain,” as that term is used herein, e.g., when referring to an RCAR, refers to an entity, typically a polypeptide-based entity, that, in the presence of a dimerization molecule, associates with another switch domain. The association results in a functional coupling of a first entity linked to, e.g., fused to, a first switch domain, and a second entity linked to, e.g., fused to, a second switch domain. A first and second switch domain are collectively referred to as a dimerization switch. In embodiments, the first and second switch domains are the same as one another, e.g., they are polypeptides having the same primary amino acid sequence, and are referred to collectively as a homodimerization switch. In embodiments, the first and second switch domains are different from one another, e.g., they are polypeptides having different primary amino acid sequences, and are referred to collectively as a heterodimerization switch. In embodiments, the switch is intracellular. In embodiments, the switch is extracellular. In embodiments, the switch domain is a polypeptide-based entity, e.g., FKBP or FRB-based, and the dimerization molecule is small molecule, e.g., a rapalogue. In embodiments, the switch domain is a polypeptide-based entity, e.g., an scFv that binds a myc peptide, and the dimerization molecule is a polypeptide, a fragment thereof, or a multimer of a polypeptide, e.g., a myc ligand or multimers of a myc ligand that bind to one or more myc scFvs. In embodiments, the switch domain is a polypeptide-based entity, e.g., myc receptor, and the dimerization molecule is an antibody or fragments thereof, e.g., myc antibody.

“Dimerization molecule,” as that term is used herein, e.g., when referring to an RCAR, refers to a molecule that promotes the association of a first switch domain with a second switch domain. In embodiments, the dimerization molecule does not naturally occur in the subject, or does not occur in concentrations that would result in significant dimerization. In embodiments, the dimerization molecule is a small molecule, e.g., rapamycin or a rapalogue, e.g., RAD001.

The term “bioequivalent” refers to an amount of an agent other than the reference compound (e.g., RAD001), required to produce an effect equivalent to the effect produced by the reference dose or reference amount of the reference compound (e.g., RAD001). In an embodiment the effect is the level of mTOR inhibition, e.g., as measured by P70 S6 kinase inhibition, e.g., as evaluated in an in vivo or in vitro assay, e.g., as measured by an assay described herein, e.g., the Boulay assay, or measurement of phosphorylated S6 levels by western blot. In an embodiment, the effect is alteration of the ratio of PD-1 positive/PD-1 negative T cells, as measured by cell sorting. In an embodiment a bioequivalent amount or dose of an mTOR inhibitor is the amount or dose that achieves the same level of P70 S6 kinase inhibition as does the reference dose or reference amount of a reference compound. In an embodiment, a bioequivalent amount or dose of an mTOR inhibitor is the amount or dose that achieves the same level of alteration in the ratio of PD-1 positive/PD-1 negative T cells as does the reference dose or reference amount of a reference compound.

The term “low, immune enhancing, dose” when used in conjunction with an mTOR inhibitor, e.g., an allosteric mTOR inhibitor, e.g., RAD001 or rapamycin, or a catalytic mTOR inhibitor, refers to a dose of mTOR inhibitor that partially, but not fully, inhibits mTOR activity, e.g., as measured by the inhibition of P70 S6 kinase activity. Methods for evaluating mTOR activity, e.g., by inhibition of P70 S6 kinase, are discussed herein. The dose is insufficient to result in complete immune suppression but is sufficient to enhance the immune response. In an embodiment, the low, immune enhancing, dose of mTOR inhibitor results in a decrease in the number of PD-1 positive immune effector cells, e.g., T cells or NK cells, and/or an increase in the number of PD-1 negative immune effector cells, e.g., T cells or NK cells, or an increase in the ratio of PD-1 negative immune effector cells (e.g., T cells or NK cells)/PD-1 positive immune effector cells (e.g., T cells or NK cells).

In an embodiment, the low, immune enhancing, dose of mTOR inhibitor results in an increase in the number of naive T cells. In an embodiment, the low, immune enhancing, dose of mTOR inhibitor results in one or more of the following:

an increase in the expression of one or more of the following markers: CD62Lhigh, CD127high, CD27+, and BCL2, e.g., on memory T cells, e.g., memory T cell precursors;

a decrease in the expression of KLRG1, e.g., on memory T cells, e.g., memory T cell precursors; and

an increase in the number of memory T cell precursors, e.g., cells with any one or combination of the following characteristics: increased CD62Lhigh, increased CD127high, increased CD27+, decreased KLRG1, and increased BCL2;

wherein any of the changes described above occurs, e.g., at least transiently, e.g., as compared to a non-treated subject.

“Progressive” as used herein refers to a disease, e.g., cancer, that is progressing or worsening. With solid tumors, e.g., lung cancer, progressive disease typically shows at least 20% growth in size or the tumor or spread of the tumor since the beginning of treatment.

“Refractory” as used herein refers to a disease, e.g., cancer, that does not respond to a treatment. In embodiments, a refractory cancer can be resistant to a treatment before or at the beginning of the treatment. In other embodiments, the refractory cancer can become resistant during a treatment. A refractory cancer is also called a resistant cancer.

“Relapsed” or a “relapse” as used herein refers to the reappearance of a disease (e.g., cancer) or the signs and symptoms of a disease such as cancer after a period of improvement or responsiveness, e.g., after prior treatment of a therapy, e.g., cancer therapy. For example, the period of responsiveness may involve the level of cancer cells falling below a certain threshold, e.g., below 20%, 15%, 10%, 5%, 4%, 3%, 2%, or 1%. The reappearance may involve the level of cancer cells rising above a certain threshold, e.g., above 20%, 1%, 10%, 5%, 4%, 3%, 2%, or 1%. More generally, in an embodiment, a response (e.g., complete response or partial response) can involve the absence of detectable MRD (minimal residual disease). In an embodiment, the initial period of responsiveness lasts at least 1, 2, 3, 4, 5, or 6 days; at least 1, 2, 3, or 4 weeks; at least 1, 2, 3, 4, 6, 8, 10, or 12 months; or at least 1, 2, 3, 4, or 5 years.

Several methods can be used to determine if a patient responds to a treatment including, for example, criteria provided by NCCN Clinical Practice Guidelines in Oncology (NCCN) Guidelines®).

A “complete response” or “CR” refers to the absence of detectable evidence of disease, e.g., cancer, e.g., a complete remission, to a treatment. A complete response may be identified, e.g., using the NCCN Guidelines® as described herein.

A “complete responder” as used herein refers to a subject having a disease, e.g., a cancer, who exhibits a complete response, e.g., a complete remission, to a treatment.

A “partial response” or “PR” refers to a decrease in the disease, e.g., cancer, although, e.g., there is still detectable disease present.

A “partial responder” as used herein refers to a subject having a disease, e.g., a cancer, who exhibits a partial response, e.g., a partial remission, to a treatment. A partial response may be identified, e.g., using the NCCN Guidelines® as described herein.

A “non-responder” as used herein refers to a subject having a disease, e.g., a cancer, who does not exhibit a response to a treatment, e.g., the patient has stable disease or progressive disease after administration of a treatment, e.g., a treatment described herein. A non-responder may be identified, e.g., using the NCCN Guidelines® as described herein.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

Various aspects of the compositions and methods herein are described in further detail below. Additional definitions are set out throughout the specification.

### DESCRIPTION

Provided herein are compositions and methods for treating a disease such as cancer, by administering a cell comprising a chimeric antigen receptor that targets an antigen, e.g., antigen described herein, e.g., EGFRvIII, e.g., EGFRvIII CAR, in combination with a PD-1 inhibitor. Exemplary components to generate a CAR molecule, e.g., EGFRvIII CAR and a CAR-expressing cell (e.g., EGFRvIII CAR-expressing cell) are disclosure herein. Exemplary PD-1 inhibitors are also described herein.

In embodiments, the combination therapy of a CAR-expressing cell (e.g., EGFRvIII CAR-expressing cell) described herein and a PD-1 inhibitor described herein results in one or more of the following: improved or increased anti-tumor activity of the CAR-expressing cell; increased proliferation or persistence of the CAR-expressing cell; improved or increased infiltration of the CAR-expressing cell; improved inhibition of tumor progression; delay of tumor progression; inhibition or reduction in cancer cell proliferation; and/or reduction in tumor burden, e.g., tumor volume, or size. In an embodiment, the combination therapy of an EGFRvIII CAR-expressing cell, e.g., a plurality of EGFRvIII CAR-expressing cells, and a PD-1 inhibitor described herein results in increased or improved persistence of an EGFRvIII CAR-expressing cell, e.g., increased or improved persistence of a plurality of EGFRvIII CAR-expressing cells.

In some embodiments, administration of the PD-1 inhibitor prior to or subsequent to administration of a CAR-expressing cell (e.g., EGFRvIII CAR-expressing cell) results in increased therapeutic efficacy, e.g., increased inhibition of tumor progression and/or tumor growth, in some cancers, e.g., as compared to administration og the PD-1 inhibitor or CAR-expressing cell alone.

PD-1 is known to downregulate the immune response, e.g., anti-tumor immune response. PD-1 and/or PD-L1 can also be expressed by cancer cells or cancer associated cells, e.g., tumor infiltrating lymphocytes (TILs). Without wishing to be bound by theory, in some embodiments, a subject that is administered the combination therapy described herein, e.g., a CAR-expressing cell (e.g., EGFRvIII CAR-expressing cell) and a PD-1 inhibitor, is more likely to have increased anti-tumor activity if the subject has one or more of: a cancer that expresses, e.g., highly expresses, PD-1 and/or PD-L1; a cancer that is infiltrated by anti-tumor immune cells, e.g., tumor infiltrating lymphocytes (TILs); and/or cancer-associated cells that express, e.g., highly express, PD-1 and/or PD-L1, as compared to a subject that is not administered the combination therapy, or is administered a CAR-expressing cell or PD-1 inhibitor alone. For example, without wishing to be bound by theory, treatment with a PD-1 inhibitor prevents or reduces the downregulation of the anti-tumor immune response, e.g., exhaustion of anti-tumor immune cells, e.g., TILs, thereby increasing the anti-tumor efficacy of the CAR-expressing cell. Without wishing to be bound by theory, administration of the combination therapy, e.g., a CAR-expressing cell, e.g., an EGFRvIII CAR-expressing cell, and an immune checkpoint inhibitor, e.g., a PD-1 inhibitor, can reduce exhaustion of T cells leading to improved, e.g., longer, persistence of CAR-expressing cells. In an embodiment, administration of a combination of an EGFRvIII CAR-expressing cell and a PD-1 inhibitor can result in improved, e.g., longer, persistence of EGFRvIII CAR-expressing cells.

### Chimeric Antigen Receptor (CAR)

The present disclosure encompasses immune effector cells (e.g., T cells or NK cells) comprising a CAR molecule that targets, e.g., specifically binds, to an antigen, e.g., antigen described herein, e.g., EGFRvIII (a CAR, e.g., EGFRvIII CAR). In one embodiment, the immune effector cells are engineered to express the CAR, e.g., EGFRvIII CAR. In one embodiment, the immune effector cells comprise a recombinant nucleic acid construct comprising nucleic acid sequences encoding the CAR, e.g., EGFRvIII CAR.

In embodiments, the CAR, e.g., EGFRvIII CAR, comprises an antigen binding domain that specifically binds to an antigen, e.g., EGFRvIII, e.g., antigen binding domain (e.g., EGFRvIII binding domain), a transmembrane domain, and an intracellular signaling domain. In one embodiment, the sequence of the antigen binding domain is contiguous with and in the same reading frame as a nucleic acid sequence encoding an intracellular signaling domain. The intracellular signaling domain can comprise a costimulatory signaling domain and/or a primary signaling domain, e.g., a zeta chain. The costimulatory signaling domain refers to a portion of the CAR comprising at least a portion of the intracellular domain of a costimulatory molecule.

The present invention encompasses a recombinant DNA construct comprising sequences encoding a CAR, wherein the CAR comprises an antibody fragment that binds specifically to EGFRvIII, e.g., a human antibody fragment that specifically binds to EGFRvIII. In one aspect, the EGFRvIII is human EGFRvIII, and the nucleic acid sequence encoding the antibody fragment is contiguous with, and in the same reading frame as a nucleic acid sequence encoding an intracellular signaling domain. The intracellular signaling domain can comprise a costimulatory signaling domain and/or a primary signaling domain, e.g., a zeta chain. The costimulatory signaling domain refers to a portion of the CAR comprising at least a portion of the intracellular domain of a costimulatory molecule.

In specific aspects, a CAR construct of the invention comprises a scFv domain selected from the group consisting of SEQ ID NO:38, SEQ ID NO:44, SEQ ID NO:50, SEQ ID NO:56, SEQ ID NO:62, SEQ ID NO:68, SEQ ID NO:74, SEQ ID NO:80, and SEQ ID NO:86, wherein the scFv may be preceded by an optional leader sequence such as provided in SEQ ID NO: 13, and followed by an optional hinge sequence such as provided in SEQ ID NO:14 or SEQ ID NO:104 or SEQ ID NO:106 or SEQ ID NO:108, a transmembrane region such as provided in SEQ ID NO:15, an intracellular signalling domain that includes SEQ ID NO:16 or SEQ ID NO:102 and a CD3 zeta sequence that includes SEQ ID NO:17 or SEQ ID NO:99, wherein the domains are contiguous with and in the same reading frame to form a single fusion protein. Also included in the invention is a nucleotide sequence that encodes the polypeptide of each of the scFv fragments selected from the group consisting of SEQ ID NO:38, SEQ ID NO:44, SEQ ID NO:50, SEQ ID NO:56, SEQ ID NO:62, SEQ ID NO:68, SEQ ID NO:74, SEQ ID NO:80, and SEQ ID NO: 86, and each of the domains of SEQ ID NOS: 13-17. Also included in the invention is a nucleotide sequence that encodes the polypeptide of each of the scFv fragments selected from the group consisting of SEQ ID NO:38, SEQ ID NO:44, SEQ ID NO:50, SEQ ID NO:56, SEQ ID NO:62, SEQ ID NO:68, SEQ ID NO:74, SEQ ID NO:80, and SEQ ID NO: 86, and each of the domains of SEQ ID NOS: 13-16 and SEQ ID NO:99. In one aspect, the EGFRvIII CAR construct comprises an optional leader sequence, an extracellular antigen binding domain that specifically binds EGFRvIII, a hinge, a transmembrane domain, and an intracellular stimulatory domain. In one aspect, the EGFRvIII CAR construct comprises an optional leader sequence, an extracellular antigen binding domain that specifically binds EGFRvIII, a hinge, a transmembrane domain, an intracellular signaling domain that includes a costimulatory domain and a primary stimulatory domain. Specific EGFRvIII CAR constructs containing a humanized scFv domain are provided in SEQ ID NO:43, SEQ ID NO:49, SEQ ID NO:55, SEQ ID NO:61, SEQ ID NO:67, SEQ ID NO:73, SEQ ID NO:79, SEQ ID NO:85, and SEQ ID NO:90. Specific EGFRvIII CAR constructs containing a murine scFv domain is provided in SEQ ID NO:1 and SEQ ID NO:2.

An exemplary leader sequence is provided as SEQ ID NO: 13. An exemplary hinge/spacer sequence is provided as SEQ ID NO: 14 or SEQ ID NO:104 or SEQ ID NO:106 or SEQ ID NO:108. An exemplary transmembrane domain sequence is provided as SEQ ID NO:15. An exemplary sequence of a costimulatory domain of the 4-1BB protein is provided as SEQ ID NO: 16. An exemplary sequence of a costimulatory domain of the CD27 protein is provided as SEQ ID NO:102. An exemplary primary signaling domain of a CD3zeta domain sequence is provided as SEQ ID NO: 17. Another exemplary primary signaling domain of a CD3zeta domain sequence is provided as SEQ ID NO:99.

In one aspect, the present invention encompasses a recombinant nucleic acid construct comprising a nucleic acid molecule encoding a CAR, wherein the nucleic acid molecule comprises the nucleic acid sequence encoding an anti-EGFRvIII binding domain, e.g., described herein, that is contiguous with, and in the same reading frame as a nucleic acid sequence encoding an intracellular signaling domain. In one aspect, the anti-EGFRvIII binding domain is selected from one or more of SEQ ID NO:38, SEQ ID NO:44, SEQ ID NO:50, SEQ ID NO:56, SEQ ID NO:62, SEQ ID NO:68, SEQ ID NO:74, SEQ ID NO:80, and SEQ ID NO:86. In one aspect, the anti-EGFRvIII binding domain is encoded by a nucleotide sequence provided in a sequence selected from the group consisting of SEQ ID NO: 39, SEQ ID NO: 45, SEQ ID NO: 51, SEQ ID NO: 57, SEQ ID NO: 63, SEQ ID NO: 69, SEQ ID NO: 75, SEQ ID NO: 81, and SEQ ID NO:98. In one aspect, the anti-EGFRvIII binding domain is encoded by SEQ ID NO: 39. In one aspect, the anti-EGFRvIII binding domain is encoded by SEQ ID NO: 45. In one aspect, the anti-EGFRvIII binding domain is encoded by SEQ ID NO: 51. In one aspect, the anti-EGFRvIII binding domain is encoded by SEQ ID NO: 57. In one aspect, the anti-EGFRvIII binding domain is encoded by SEQ ID NO: 63. In one aspect, the anti-EGFRvIII binding domain is encoded by SEQ ID NO: 69. In one aspect, the anti-EGFRvIII binding domain is encoded by SEQ ID NO: 75. In one aspect, the anti-EGFRvIII binding domain is encoded by SEQ ID NO: 81.

In one aspect, the present invention encompasses a recombinant nucleic acid construct comprising a nucleic acid molecule encoding a CAR, wherein the nucleic acid molecule comprises a nucleic acid sequence encoding an anti-EGFRvIII binding domain selected from the group consisting of SEQ ID NO:42, SEQ ID NO:48, SEQ ID NO:54, SEQ ID NO:60, SEQ ID NO:66, SEQ ID NO:72, SEQ ID NO:78, SEQ ID NO:84, and SEQ ID NO:90 wherein the sequence is contiguous with and in the same reading frame as the nucleic acid sequence encoding an intracellular signaling domain. An exemplary intracellular signaling domain that can be used in the CAR includes, but is not limited to, one or more intracellular signaling domains of, e.g., CD3-zeta, CD28, 4-1BB, and the like. In some instances, the CAR can comprise any combination of intracellular signaling domains of CD3-zeta, CD28, 4-1BB, and the like. In one aspect the nucleic acid construct comprises SEQ ID NO: 42. In one aspect the nucleic acid sequence of a CAR construct is SEQ ID NO:48. In one aspect the nucleic acid construct comprises SEQ ID NO:54. In one aspect the nucleic acid construct comprises SEQ ID NO:60. In one aspect the nucleic acid construct comprises SEQ ID NO:66. In one aspect the nucleic acid construct comprises SEQ ID NO:72. In one aspect the nucleic acid construct comprises SEQ ID NO:78. In one aspect the nucleic acid construct comprises SEQ ID NO:84.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the nucleic acid of interest can be produced synthetically, rather than cloned.

The present invention includes retroviral and lentiviral vector constructs expressing a CAR that can be directly transduced into a cell.

The present invention also includes an RNA construct that can be directly transfected into a cell. A method for generating mRNA for use in transfection involves in vitro transcription (IVT) of a template with specially designed primers, followed by polyA addition, to produce a construct containing 3′ and 5′ untranslated sequence (“UTR”), a 5′ cap and/or Internal Ribosome Entry Site (IRES), the nucleic acid to be expressed, and a polyA tail, typically 50-2000 bases (SEQ ID NO: 116) in length. RNA so produced can efficiently transfect different kinds of cells. In one embodiment, the template includes sequences for the CAR. In an embodiment, an RNA CAR vector is transduced into a T cell by electroporation.

### Antigen Binding Domain

In one aspect, the CAR of the invention comprises a target-specific binding element otherwise referred to as an antigen binding domain. The choice of moiety depends upon the type and number of ligands that define the surface of a target cell. For example, the antigen binding domain may be chosen to recognize a ligand that acts as a cell surface marker on target cells associated with a particular disease state. Thus examples of cell surface markers that may act as ligands for the antigen binding domain in a CAR of the invention include those associated with viral, bacterial and parasitic infections, autoimmune disease and cancer cells. In one aspect, the CAR-mediated T-cell response can be directed to an antigen of interest by way of engineering an antigen binding domain that specifically binds a desired antigen into the CAR.

The antigen binding domain can be any domain that binds to the antigen including but not limited to a monoclonal antibody, a polyclonal antibody, a recombinant antibody, a human antibody, a humanized antibody, and a functional fragment thereof, including but not limited to a single-domain antibody such as a heavy chain variable domain (VH), a light chain variable domain (VL) and a variable domain (VHH) of camelid derived nanobody, and to an alternative scaffold known in the art to function as an antigen binding domain, such as a recombinant fibronectin domain, and the like. In some instances, it is beneficial for the antigen binding domain to be derived from the same species in which the CAR will ultimately be used in. For example, for use in humans, it may be beneficial for the antigen binding domain of the CAR to comprise human or humanized residues for the antigen binding domain of an antibody or antibody fragment. Thus, in one aspect, the antigen binding domain comprises a human antibody or an antibody fragment.

In one embodiment, the portion of the CAR comprising the antigen binding domain comprises an antigen binding domain that targets, e.g., specifically binds to, an antigen, e.g., antigen described herein, e.g., EGFRvIII. In one embodiment, the antigen binding domain targets, e.g., specifically binds to, human EGFRvIII.

For example, a mouse monoclonal antibody (IgG2b) 3C10 was produced against EGFRvIII by immunization of mice with a 14 amino acid peptide (LEEKKGNYVVTDHC; SEQ ID NO:101) including the EGFRvIII-specific fusion junction and demonstrated highly specific recognition of EGFRvIII without any detectable binding to wild-type EGFR (Okamoto et al, British J. Cancer 1996, 73:1366-1372). Accordingly, in some embodiments, the antigen binding domain targets an amino acid sequence, e.g., an amino acid sequence comprising an added glycine residue, within the EGFvIII fusion junction domain. In some embodiments, the antigen binding domain targets an one or more amino acid sequence in the amino acid sequence of SEQ ID NO:101.

The antigen binding domain can be any domain that binds to the antigen including, but not limited to, a monoclonal antibody, a polyclonal antibody, a recombinant antibody, a human antibody, a humanized antibody, and a functional fragment thereof, including but not limited to a single-domain antibody such as a heavy chain variable domain (VH), a light chain variable domain (VL) and a variable domain (VHH) of camelid derived nanobody, and to an alternative scaffold known in the art to function as antigen binding domain, such as a recombinant fibronectin domain, and the like. In some instances, it is beneficial for the antigen binding domain to be derived from the same species in which the CAR will ultimately be used in. For example, for use in humans, it may be beneficial for the antigen binding domain of the CAR to comprise human or humanized residues for the antigen binding domain of an antibody or antibody fragment.

Thus, in one aspect, the antigen binding domain comprises a human antibody or an antibody fragment. In another aspect, the antigen binding domain comprises a humanized antibody or antibody fragment. In one embodiment, the anti-EGFRvIII binding domain comprises one or more (e.g., one, two, or all three) light chain complementary determining region 1 (LC CDR1), light chain complementary determining region 2 (LC CDR2), and light chain complementary determining region 3 (LC CDR3) of an anti-EGFRvIII binding domain described herein, and one or more (e.g., one, two, or all three) heavy chain complementary determining region 1 (HC CDR1), heavy chain complementary determining region 2 (HC CDR2), and heavy chain complementary determining region 3 (HC CDR3) of an anti-EGFRvIII binding domain described herein. In one embodiment, the anti-EGFRvIII binding domain comprises a light chain variable region described herein and/or a heavy chain variable region described herein. In one embodiment, the anti-EGFRvIII binding domain is a scFv comprising a light chain variable region and a heavy chain variable region of an amino acid sequence, e.g., a light chain variable region and heavy chain variable region described herein. In an embodiment, the anti-EGFRvIII binding domain (e.g., an scFv) comprises: a light chain variable region comprising an amino acid sequence having at least one, two or three modifications (e.g., substitutions) but not more than 30, 20 or 10 modifications (e.g., substitutions) of an amino acid sequence of a light chain variable region provided herein, or a sequence with 85-99% (e.g., 90-99%, or 95-99%) identity to an amino acid sequence provided herein; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided herein, or a sequence with 85-99% (e.g., 90-99%, or 95-99%) identity to an amino acid sequence provided herein. In one aspect, the antigen binding domain comprises one or more sequence selected from the group consisting of SEQ ID NO:38, SEQ ID NO:44, SEQ ID NO:50, SEQ ID NO:56, SEQ ID NO:62, SEQ ID NO:68, SEQ ID NO:74, SEQ ID NO:80, and SEQ ID NO:86. In one aspect the humanized CAR is selected from one or more sequence selected from the group consisting of SEQ ID NO:43, SEQ ID NO:49, SEQ ID NO:55, SEQ ID NO:61, SEQ ID NO:67, SEQ ID NO:73, SEQ ID NO:79, SEQ ID NO:85, and SEQ ID NO:90.

In some aspects, a non-human antibody is humanized, where specific sequences or regions of the antibody are modified to increase similarity to an antibody naturally produced in a human or fragment thereof. In one aspect, the antigen binding domain is humanized.

A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., 2002, J. Immunol., 169:1119-25; Caldas et al., 2000, Protein Eng., 13(5):353-60; Morea et al., 2000, Methods, 20:267-79; Baca et al., 1997, J. Biol. Chem., 272:10678-84; Roguska et al., 1996, Protein Eng., 9(10):895-904; Couto et al., 1995, Cancer Res., 55:5973s-5977; Couto et al., 1995, Cancer Res., 55(8):1717-22; Sandhu 1994 Gene, 150(2):409-10; and Pedersen et al., 1994, J. Mol. Biol., 235(3):959-73, each of which is incorporated herein in its entirety by reference. Often, framework residues in the framework regions will be substituted with the corresponding residue from the CDR donor antibody to alter, for example improve, antigen binding. These framework substitutions are identified by methods well-known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature, 332:323, which are incorporated herein by reference in their entireties.)

A humanized antibody or antibody fragment has one or more amino acid residues remaining in it from a source which is nonhuman. These nonhuman amino acid residues are often referred to as “import” residues, which are typically taken from an “import” variable domain. As provided herein, humanized antibodies or antibody fragments comprise one or more CDRs from nonhuman immunoglobulin molecules and framework regions wherein the amino acid residues comprising the framework are derived completely or mostly from human germline. Multiple techniques for humanization of antibodies or antibody fragments are well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated herein by reference herein in their entirety). In such humanized antibodies and antibody fragments, substantially less than an intact human variable domain has been substituted by the corresponding sequence from a nonhuman species. Humanized antibodies are often human antibodies in which some CDR residues and possibly some framework (FR) residues are substituted by residues from analogous sites in rodent antibodies. Humanization of antibodies and antibody fragments can also be achieved by veneering or resurfacing (EP 592,106; EP 519,596; Padlan, 1991, Molecular Immunology, 28(4/5):489-498; Studnicka et al., Protein Engineering, 7(6):805-814 (1994); and Roguska et al., PNAS, 91:969-973 (1994)) or chain shuffling (U.S. Pat. No. 5,565,332), the contents of which are incorporated herein by reference herein in their entirety.

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987), the contents of which are incorporated herein by reference herein in their entirety). Another method uses a particular framework derived from the consensus sequence of all human antibodies of a particular subgroup of light or heavy chains. The same framework may be used for several different humanized antibodies (see, e.g., Carter et al., Proc. Natl. Acad. Sci. USA, 89:4285 (1992); Presta et al., J. Immunol., 151:2623 (1993), the contents of which are incorporated herein by reference herein in their entirety).

In some aspects, the portion of a CAR composition of the invention that comprises an antibody fragment is humanized with retention of high affinity for the target antigen and other favorable biological properties. According to one aspect of the invention, humanized antibodies and antibody fragments are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, e.g., the analysis of residues that influence the ability of the candidate immunoglobulin to bind the target antigen. In this way, FR residues can be selected and combined from the recipient and import sequences so that the desired antibody or antibody fragment characteristic, such as increased affinity for the target antigen, is achieved. In general, the CDR residues are directly and most substantially involved in influencing antigen binding.

In one aspect, the anti-EGFRvIII binding domain is, for example, a Fv, a Fab, or a (Fab′)2, or a bi-functional (e.g. bi-specific) hybrid antibody (e.g., Lanzavecchia et al., Eur. J. Immunol. 17, 105 (1987)). In one aspect, an antibody fragment provided herein is a scFv. In one aspect, the scFv binds an EGFRvIII protein but not wild type EGFR. In some instances, a human scFv may also be derived from a yeast display library.

In some instances, scFvs can be prepared according to method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise a linker (e.g., a Ser-Gly linker) with an optimized length and/or amino acid composition. The linker length can greatly affect how the variable regions of an scFv fold and interact. In fact, if a short polypeptide linker is employed (e.g., between 5-10 amino acids, intrachain folding is prevented. Interchain folding is also required to bring the two variable regions together to form a functional epitope binding site. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, is incorporated herein by reference.

An scFv can comprise a linker of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally occurring amino acid. In some embodiments, the linker sequence comprises amino acids glycine and serine. In another embodiment, the linker sequence comprises sets of glycine and serine repeats such as (Gly₄Ser)n (SEQ ID NO: 37), where n is a positive integer equal to or greater than 1. In one embodiment, the linker can be (Gly₄Ser)₄ (SEQ ID NO: 113) or (Gly₄Ser)₃ (SEQ ID NO: 114). Variation in the linker length may retain or enhance activity, giving rise to superior efficacy in activity studies.

Stability and Mutations

The stability of an anti-EGFRvIII binding domain, e.g., scFv molecules (e.g., soluble scFv), can be evaluated in reference to the biophysical properties (e.g., thermal stability) of a conventional control scFv molecule or a full length antibody. In one embodiment, the humanized scFv has a thermal stability that is greater than about 0.1, about 0.25, about 0.5, about 0.75, about 1, about 1.25, about 1.5, about 1.75, about 2, about 2.5, about 3, about 3.5, about 4, about 4.5, about 5, about 5.5, about 6, about 6.5, about 7, about 7.5, about 8, about 8.5, about 9, about 9.5, about 10 degrees, about 11 degrees, about 12 degrees, about 13 degrees, about 14 degrees, or about 15 degrees Celsius than a control binding molecule (e.g. a conventional scFv molecule) in the described assays.

The improved thermal stability of the anti-EGFRvIII binding domain, e.g., scFv, is subsequently conferred to the entire EGFRvIII CAR construct, leading to improved therapeutic properties of the EGFRvIII CAR construct. The thermal stability of the anti-EGFRvIII binding domain, e.g., scFv, can be improved by at least about 2° C. or 3° C. as compared to a conventional antibody. In one embodiment, the anti-EGFRvIII binding domain, e.g., scFv, has a 1° C. improved thermal stability as compared to a conventional antibody. In another embodiment, the anti-EGFRvIII binding domain, e.g., scFv, has a 2° C. improved thermal stability as compared to a conventional antibody. In another embodiment, the anti-EGFRvIII binding domain, e.g., scFv, has a 4, 5, 6, 7, 8, 9, 10, 11, 12, 13, 14, 15° C. improved thermal stability as compared to a conventional antibody. Comparisons can be made, for example, between the scFv molecules disclosed herein and scFv molecules or Fab fragments of an antibody from which the scFv VH and VL were derived. Thermal stability can be measured using methods known in the art. For example, in one embodiment, Tm can be measured. Methods for measuring Tm and other methods of determining protein stability are described in more detail below.

Mutations in scFv (arising through humanization or direct mutagenesis of the soluble scFv) alter the stability of the scFv and improve the overall stability of the scFv and the EGFRvIII CAR construct. Stability of the humanized scFv is compared against the murine scFv using measurements such as Tm, temperature denaturation and temperature aggregation. The binding capacity of the mutant scFvs can be determined using assays described in the Examples.

In one embodiment, the anti-EGFRvIII binding domain, e.g., scFv, comprises at least one mutation arising from the humanization process such that the mutated scFv confers improved stability to the EGFRvIII construct. In another embodiment, the anti-EGFRvIII binding domain, e.g., scFv, comprises at least 1, 2, 3, 4, 5, 6, 7, 8, 9, 10 mutations arising from the humanization process such that the mutated scFv confers improved stability to the EGFRvIII construct.

Methods of Evaluating Stability

The stability of an antigen binding domain may be assessed using, e.g., the methods described below. Such methods allow for the determination of multiple thermal unfolding transitions where the least stable domain either unfolds first or limits the overall stability threshold of a multidomain unit that unfolds cooperatively (e.g. a multidomain protein which exhibits a single unfolding transition). The least stable domain can be identified in a number of additional ways. Mutagenesis can be performed to probe which domain limits the overall stability. Additionally, protease resistance of a multidomain protein can be performed under conditions where the least stable domain is known to be intrinsically unfolded via DSC or other spectroscopic methods (Fontana, et al., (1997) Fold. Des., 2: R17-26; Dimasi et al. (2009) J. Mol. Biol. 393: 672-692). Once the least stable domain is identified, the sequence encoding this domain (or a portion thereof) may be employed as a test sequence in the methods.

a) Thermal Stability

The thermal stability of the compositions may be analyzed using a number of non-limiting biophysical or biochemical techniques known in the art. In certain embodiments, thermal stability is evaluated by analytical spectroscopy.

An exemplary analytical spectroscopy method is Differential Scanning calorimetry (DSC). DSC employs a calorimeter which is sensitive to the heat absorbances that accompany the unfolding of most proteins or protein domains (see, e.g. Sanchez-Ruiz, et al., Biochemistry, 27: 1648-52, 1988). To determine the thermal stability of a protein, a sample of the protein is inserted into the calorimeter and the temperature is raised until the Fab or scFv unfolds. The temperature at which the protein unfolds is indicative of overall protein stability.

Another exemplary analytical spectroscopy method is Circular Dichroism (CD) spectroscopy. CD spectrometry measures the optical activity of a composition as a function of increasing temperature. Circular dichroism (CD) spectroscopy measures differences in the absorption of left-handed polarized light versus right-handed polarized light which arise due to structural asymmetry. A disordered or unfolded structure results in a CD spectrum very different from that of an ordered or folded structure. The CD spectrum reflects the sensitivity of the proteins to the denaturing effects of increasing temperature and is therefore indicative of a protein's thermal stability (see van Mierlo and Steemsma, J. Biotechnol., 79(3):281-98, 2000).

Another exemplary analytical spectroscopy method for measuring thermal stability is Fluorescence Emission Spectroscopy (see van Mierlo and Steemsma, supra). Yet another exemplary analytical spectroscopy method for measuring thermal stability is Nuclear Magnetic Resonance (NMR) spectroscopy (see, e.g. van Mierlo and Steemsma, supra).

The thermal stability of a composition can be measured biochemically. An exemplary biochemical method for assessing thermal stability is a thermal challenge assay. In a “thermal challenge assay”, a composition is subjected to a range of elevated temperatures for a set period of time. For example, in one embodiment, test scFv molecules or molecules comprising scFv molecules are subject to a range of increasing temperatures, e.g., for 1-1.5 hours. The activity of the protein is then assayed by a relevant biochemical assay. For example, if the protein is a binding protein (e.g. an scFv or scFv-containing polypeptide) the binding activity of the binding protein may be determined by a functional or quantitative ELISA.

Such an assay may be done in a high-throughput format and those disclosed in the Examples using E. coli and high throughput screening. A library of anti-EGFRvIII binding domain, e.g., scFv, variants may be created using methods known in the art. Anti-EGFRvIII binding domain, e.g., scFv, expression may be induced and the anti-EGFRvIII binding domain, e.g., scFv, may be subjected to thermal challenge. The challenged test samples may be assayed for binding and those anti-EGFRvIII binding domains, e.g., scFvs, which are stable may be scaled up and further characterized.

Thermal stability is evaluated by measuring the melting temperature (Tm) of a composition using any of the above techniques (e.g. analytical spectroscopy techniques). The melting temperature is the temperature at the midpoint of a thermal transition curve wherein 50% of molecules of a composition are in a folded state (See e.g., Dimasi et al. (2009) J. Mol Biol. 393: 672-692). In one embodiment, Tm values for an anti-EGFRvIII binding domain, e.g., scFv, are about 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., 100° C. In one embodiment, Tm values for an IgG is about 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., 100° C. In one embodiment, Tm values for an multivalent antibody is about 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., 100° C.

Thermal stability is also evaluated by measuring the specific heat or heat capacity (Cp) of a composition using an analytical calorimetric technique (e.g. DSC). The specific heat of a composition is the energy (e.g. in kcal/mol) is required to rise by 1° C., the temperature of 1 mol of water. As large Cp is a hallmark of a denatured or inactive protein composition. The change in heat capacity (□Cp) of a composition is measured by determining the specific heat of a composition before and after its thermal transition. Thermal stability may also be evaluated by measuring or determining other parameters of thermodynamic stability including Gibbs free energy of unfolding (□G), enthalpy of unfolding (□H), or entropy of unfolding (□S). One or more of the above biochemical assays (e.g. a thermal challenge assay) are used to determine the temperature (i.e. the Tc value) at which 50% of the composition retains its activity (e.g. binding activity).

In addition, mutations to the anti-EGFRvIII binding domain, e.g., scFv, alter the thermal stability of the anti-EGFRvIII binding domain, e.g., scFv, compared with the unmutated anti-EGFRvIII binding domain, e.g., scFv. When the humanized anti-EGFRvIII binding domain, e.g., scFv, is incorporated into an anti-EGFRvIII CAR construct, the anti-EGFRvIII binding domain, e.g., humanized scFv confers thermal stability to the overall anti-EGFRvIII CAR construct. In one embodiment, the anti-EGFRvIII binding domain, e.g., scFv, comprises a single mutation that confers thermal stability to the anti-EGFRvIII binding domain, e.g., scFv. In another embodiment, the anti-EGFRvIII binding domain, e.g., scFv, comprises multiple mutations that confer thermal stability to the anti-EGFRvIII binding domain, e.g., scFv. In one embodiment, the multiple mutations in the anti-EGFRvIII binding domain, e.g., scFv, have an additive effect on thermal stability of the anti-EGFRvIII binding domain, e.g., scFv.

b) % Aggregation

The stability of a composition can be determined by measuring its propensity to aggregate. Aggregation can be measured by a number of non-limiting biochemical or biophysical techniques. For example, the aggregation of a composition may be evaluated using chromatography, e.g. Size-Exclusion Chromatography (SEC). SEC separates molecules on the basis of size. A column is filled with semi-solid beads of a polymeric gel that will admit ions and small molecules into their interior but not large ones. When a protein composition is applied to the top of the column, the compact folded proteins (i.e. non-aggregated proteins) are distributed through a larger volume of solvent than is available to the large protein aggregates. Consequently, the large aggregates move more rapidly through the column, and in this way the mixture can be separated or fractionated into its components. Each fraction can be separately quantified (e.g. by light scattering) as it elutes from the gel. Accordingly, the % aggregation of a composition can be determined by comparing the concentration of a fraction with the total concentration of protein applied to the gel. Stable compositions elute from the column as essentially a single fraction and appear as essentially a single peak in the elution profile or chromatogram.

c) Binding Affinity

The stability of a composition can be assessed by determining its target binding affinity. A wide variety of methods for determining binding affinity are known in the art. An exemplary method for determining binding affinity employs surface plasmon resonance. Surface plasmon resonance is an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Jonsson, U., et al. (1993) Ann. Biol. Clin. 51:19-26; Jonsson, U., i (1991) Biotechniques 11:620-627; Johnsson, B., et al. (1995) J. Mol. Recognit. 8:125-131; and Johnnson, B., et al. (1991) Anal. Biochem. 198:268-277.

In one aspect, the antigen binding domain of the CAR comprises an amino acid sequence that is homologous to an antigen binding domain amino acid sequence described herein, and the antigen binding domain retains the desired functional properties of the anti-EGFRvIII antibody fragments described herein. In one specific aspect, the CAR composition of the invention comprises an antibody fragment. In a further aspect, that antibody fragment comprises an scFv.

In various aspects, the antigen binding domain of the CAR is engineered by modifying one or more amino acids within one or both variable regions (e.g., VH and/or VL), for example within one or more CDR regions and/or within one or more framework regions. In one specific aspect, the CAR composition of the invention comprises an antibody fragment. In a further aspect, that antibody fragment comprises an scFv.

It will be understood by one of ordinary skill in the art that the antibody or antibody fragment of the invention may further be modified such that they vary in amino acid sequence (e.g., from wild-type), but not in desired activity. For example, additional nucleotide substitutions leading to amino acid substitutions at “non-essential” amino acid residues may be made to the protein. For example, a nonessential amino acid residue in a molecule may be replaced with another amino acid residue from the same side chain family. In another embodiment, a string of amino acids can be replaced with a structurally similar string that differs in order and/or composition of side chain family members, e.g., a conservative substitution, in which an amino acid residue is replaced with an amino acid residue having a similar side chain, may be made.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Percent identity in the context of two or more nucleic acids or polypeptide sequences, refers to two or more sequences that are the same. Two sequences are “substantially identical” if two sequences have a specified percentage of amino acid residues or nucleotides that are the same (e.g., 60% identity, optionally 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well known in the art. Optimal alignment of sequences for comparison can be conducted, e.g., by the local homology algorithm of Smith and Waterman, (1970) Adv. Appl. Math. 2:482c, by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., (2003) Current Protocols in Molecular Biology).

Two examples of algorithms that are suitable for determining percent sequence identity and sequence similarity are the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information.

The percent identity between two amino acid sequences can also be determined using the algorithm of E. Meyers and W. Miller, (1988) Comput. Appl. Biosci. 4:11-17) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (1970) J. Mol. Biol. 48:444-453) algorithm which has been incorporated into the GAP program in the GCG software package (available at www.gcg.com), using either a Blossom 62 matrix or a PAM250 matrix, and a gap weight of 16, 14, 12, 10, 8, 6, or 4 and a length weight of 1, 2, 3, 4, 5, or 6.

In one aspect, the present invention contemplates modifications of the starting antibody or fragment (e.g., scFv) amino acid sequence that generate functionally equivalent molecules.

For example, the VH or VL of an anti-EGFRvIII binding domain, e.g., scFv, comprised in the CAR can be modified to retain at least about 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity of the starting VH or VL framework region of the anti-EGFRvIII binding domain, e.g., scFv. The present invention contemplates modifications of the entire CAR construct, e.g., modifications in one or more amino acid sequences of the various domains of the CAR construct in order to generate functionally equivalent molecules. The CAR construct can be modified to retain at least about 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity of the starting CAR construct.

Bispecific CARs

In an embodiment a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment a bispecific antibody molecule comprises a half antibody having binding specificity for a first epitope and a half antibody having binding specificity for a second epitope. In an embodiment a bispecific antibody molecule comprises a half antibody, or fragment thereof, having binding specificity for a first epitope and a half antibody, or fragment thereof, having binding specificity for a second epitope. In an embodiment a bispecific antibody molecule comprises a scFv, or fragment thereof, have binding specificity for a first epitope and a scFv, or fragment thereof, have binding specificity for a second epitope.

In certain embodiments, the antibody molecule is a multi-specific (e.g., a bispecific or a trispecific) antibody molecule. Protocols for generating bispecific or heterodimeric antibody molecules are known in the art; including but not limited to, for example, the “knob in a hole” approach described in, e.g., U.S. Pat. No. 5,731,168; the electrostatic steering Fc pairing as described in, e.g., WO 09/089004, WO 06/106905 and WO 2010/129304; Strand Exchange Engineered Domains (SEED) heterodimer formation as described in, e.g., WO 07/110205; Fab arm exchange as described in, e.g., WO 08/119353, WO 2011/131746, and WO 2013/060867; double antibody conjugate, e.g., by antibody cross-linking to generate a bi-specific structure using a heterobifunctional reagent having an amine-reactive group and a sulfhydryl reactive group as described in, e.g., U.S. Pat. No. 4,433,059; bispecific antibody determinants generated by recombining half antibodies (heavy-light chain pairs or Fabs) from different antibodies through cycle of reduction and oxidation of disulfide bonds between the two heavy chains, as described in, e.g., U.S. Pat. No. 4,444,878; trifunctional antibodies, e.g., three Fab′ fragments cross-linked through sulfhydryl reactive groups, as described in, e.g., U.S. Pat. No. 5,273,743; biosynthetic binding proteins, e.g., pair of scFvs cross-linked through C-terminal tails preferably through disulfide or amine-reactive chemical cross-linking, as described in, e.g., U.S. Pat. No. 5,534,254; bifunctional antibodies, e.g., Fab fragments with different binding specificities dimerized through leucine zippers (e.g., c-fos and c-jun) that have replaced the constant domain, as described in, e.g., U.S. Pat. No. 5,582,996; bispecific and oligospecific mono- and oligovalent receptors, e.g., VH-CH1 regions of two antibodies (two Fab fragments) linked through a polypeptide spacer between the CH1 region of one antibody and the VH region of the other antibody typically with associated light chains, as described in, e.g., U.S. Pat. No. 5,591,828; bispecific DNA-antibody conjugates, e.g., crosslinking of antibodies or Fab fragments through a double stranded piece of DNA, as described in, e.g., U.S. Pat. No. 5,635,602; bispecific fusion proteins, e.g., an expression construct containing two scFvs with a hydrophilic helical peptide linker between them and a full constant region, as described in, e.g., U.S. Pat. No. 5,637,481; multivalent and multispecific binding proteins, e.g., dimer of polypeptides having first domain with binding region of Ig heavy chain variable region, and second domain with binding region of Ig light chain variable region, generally termed diabodies (higher order structures are also encompassed creating for bispecific, trispecific, or tetraspecific molecules, as described in, e.g., U.S. Pat. No. 5,837,242; minibody constructs with linked VL and VH chains further connected with peptide spacers to an antibody hinge region and CH3 region, which can be dimerized to form bispecific/multivalent molecules, as described in, e.g., U.S. Pat. No. 5,837,821; VH and VL domains linked with a short peptide linker (e.g., 5 or 10 amino acids) or no linker at all in either orientation, B which can form dimers to form bispecific diabodies; trimers and tetramers, as described in, e.g., U.S. Pat. No. 5,844,094; String of VH domains (or VL domains in family members) connected by peptide linkages with crosslinkable groups at the C-terminus further associated with VL domains to form a series of FVs (or scFvs), as described in, e.g., U.S. Pat. No. 5,864,019; and single chain binding polypeptides with both a VH and a VL domain linked through a peptide linker are combined into multivalent structures through non-covalent or chemical crosslinking to form, e.g., homobivalent, heterobivalent, trivalent, and tetravalent structures using both scFV or diabody type format, as described in, e.g., U.S. Pat. No. 5,869,620. Additional exemplary multispecific and bispecific molecules and methods of making the same are found, for example, in U.S. Pat. Nos. 5,910,573, 5,932,448, 5,959,083, 5,989,830, 6,005,079, 6,239,259, 6,294,353, 6,333,396, 6,476,198, 6,511,663, 6,670,453, 6,743,896, 6,809,185, 6,833,441, 7,129,330, 7,183,076, 7,521,056, 7,527,787, 7,534,866, 7,612,181, US2002004587A1, US2002076406A1, US2002103345A1, US2003207346A1, US2003211078A1, US2004219643A1, US2004220388A1, US2004242847A1, US2005003403A1, US2005004352A1, US2005069552A1, US2005079170A1, US2005100543A1, US2005136049A1, US2005136051A1, US2005163782A1, US2005266425A1, US2006083747A1, US2006120960A1, US2006204493A1, US2006263367A1, US2007004909A1, US2007087381A1, US2007128150A1, US2007141049A1, US2007154901A1, US2007274985A1, US2008050370A1, US2008069820A1, US2008152645A1, US2008171855A1, US2008241884A1, US2008254512A1, US2008260738A1, US2009130106A1, US2009148905A1, US2009155275A1, US2009162359A1, US2009162360A1, US2009175851A1, US2009175867A1, US2009232811A1, US2009234105A1, US2009263392A1, US2009274649A1, EP346087A2, WO0006605A2, WO02072635A2, WO04081051A1, WO06020258A2, WO2007044887A2, WO2007095338A2, WO2007137760A2, WO2008119353A1, WO2009021754A2, WO2009068630A1, WO9103493A1, WO9323537A1, WO9409131A1, WO9412625A2, WO9509917A1, WO9637621A2, WO9964460A1. The contents of the above-referenced applications are incorporated herein by reference in their entireties.

Within each antibody or antibody fragment (e.g., scFv) of a bispecific antibody molecule, the VH can be upstream or downstream of the VL. In some embodiments, the upstream antibody or antibody fragment (e.g., scFv) is arranged with its VH (VH₁) upstream of its VL (VL₁) and the downstream antibody or antibody fragment (e.g., scFv) is arranged with its VL (VL₂) upstream of its VH (VH₂), such that the overall bispecific antibody molecule has the arrangement VH₁-VL₁-VL₂-VH₂. In other embodiments, the upstream antibody or antibody fragment (e.g., scFv) is arranged with its VL (VL₁) upstream of its VH (VH₁) and the downstream antibody or antibody fragment (e.g., scFv) is arranged with its VH (VH₂) upstream of its VL (VL₂), such that the overall bispecific antibody molecule has the arrangement VL₁-VH₁-VH₂-VL₂. Optionally, a linker is disposed between the two antibodies or antibody fragments (e.g., scFvs), e.g., between VL₁ and VL₂ if the construct is arranged as VH₁-VL₁-VL₂-VH₂, or between VH₁ and VH₂ if the construct is arranged as VL₁-VH₁-VH₂-VL₂. The linker may be a linker as described herein, e.g., a (Gly₄-Ser)n linker, wherein n is 1, 2, 3, 4, 5, or 6, preferably 4 (SEQ ID NO: 110). In general, the linker between the two scFvs should be long enough to avoid mispairing between the domains of the two scFvs. Optionally, a linker is disposed between the VL and VH of the first scFv. Optionally, a linker is disposed between the VL and VH of the second scFv. In constructs that have multiple linkers, any two or more of the linkers can be the same or different. Accordingly, in some embodiments, a bispecific CAR comprises VLs, VHs, and optionally one or more linkers in an arrangement as described herein.

In certain embodiments the antibody molecule is a bispecific antibody molecule having a first binding specificity for EGFRvIII and a second binding specificity for an antigen other than EGFRvIII.

In one aspect, the EGFRvIII antibodies and antibody fragments of the present invention (for example, those disclosed in Table 2) can be grafted to one or more constant domain of a T cell receptor (“TCR”) chain, for example, a TCR alpha or TCR beta chain, to create an chimeric TCR that binds specificity to EGFRvIII. Without being bound by theory, it is believed that chimeric TCRs will signal through the TCR complex upon antigen binding. For example, an EGFRvIII scFv as disclosed herein, can be grafted to the constant domain, e.g., at least a portion of the extracellular constant domain, the transmembrane domain and the cytoplasmic domain, of a TCR chain, for example, the TCR alpha chain and/or the TCR beta chain. As another example, an EGFRvIII antibody fragment, for example a VL domain as described herein, can be grafted to the constant domain of a TCR alpha chain, and an EGFRvIII antibody fragment, for example a VH domain as described herein, can be grafted to the constant domain of a TCR beta chain (or alternatively, a VL domain may be grafted to the constant domain of the TCR beta chain and a VH domain may be grafted to a TCR alpha chain). As another example, the CDRs of an EGFRvIII antibody or antibody fragment, e.g., the CDRs of an EGFRvIII antibody or antibody fragment as described in Table 2 may be grafted into a TCR alpha and/or beta chain to create a chimeric TCR that binds specifically to EGFRvIII. For example, the LCDRs disclosed herein may be grafted into the variable domain of a TCR alpha chain and the HCDRs disclosed herein may be grafted to the variable domain of a TCR beta chain, or vice versa. Such chimeric TCRs may be produced by methods known in the art (For example, Willemsen R A et al, Gene Therapy 2000; 7: 1369-1377; Zhang T et al, Cancer Gene Ther 2004; 11: 487-496; Aggen et al, Gene Ther. 2012 April; 19(4):365-74).

### Transmembrane Domain

With respect to the transmembrane domain, in various embodiments, a CAR can be designed to comprise a transmembrane domain that is attached to the extracellular domain of the CAR. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In one aspect, the transmembrane domain is one that is associated with one of the other domains of the CAR is used. In some instances, the transmembrane domain can be selected or modified by amino acid substitution to avoid binding of such domains to the transmembrane domains of the same or different surface membrane proteins, e.g., to minimize interactions with other members of the receptor complex. In one aspect, the transmembrane domain is capable of homodimerization with another CAR on the CART cell surface. In a different aspect, the amino acid sequence of the transmembrane domain may be modified or substituted so as to minimize interactions with the binding domains of the native binding partner present in the same CART.

The transmembrane domain may be derived either from a natural or from a recombinant source. Where the source is natural, the domain may be derived from any membrane-bound or transmembrane protein. In one aspect the transmembrane domain is capable of signaling to the intracellular domain(s) whenever the CAR has bound to a target. A transmembrane domain of particular use in this invention may include at least the transmembrane region(s) of e.g., the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154.

In some instances, the transmembrane domain can be attached to the extracellular region of the CAR, e.g., the antigen binding domain of the CAR, via a hinge, e.g., a hinge from a human protein. For example, in one embodiment, the hinge can be a human Ig (immunoglobulin) hinge, e.g., an IgG4 hinge, or a CD8a hinge. In one embodiment, the hinge or spacer comprises (e.g., consists of) the amino acid sequence of SEQ ID NO:14. In one aspect, the transmembrane domain comprises (e.g., consists of) a transmembrane domain of SEQ ID NO: 15.

In one aspect, the hinge or spacer comprises an IgG4 hinge. For example, in one embodiment, the hinge or spacer comprises a hinge of the amino acid sequence ESKYGPPCPPCPAPEFLGGPSVFLFPPKPKDTLMISRTPEVTCVVVDVSQEDPEVQFNWY VDGVEVHNAKTKPREEQFNSTYRVVSVLTVLHQDWLNGKEYKCKVSNKGLPSSIEKTIS KAKGQPREPQVYTLPPSQEEMTKNQVSLTCLVKGFYPSDIAVEWESNGQPENNYKTTPP VLDSDGSFFLYSRLTVDKSRWQEGNVFSCSVMHEALHNHYTQKSLSLSLGKM (SEQ ID NO:104). In some embodiments, the hinge or spacer comprises a hinge encoded by a nucleotide sequence of

(SEQ ID NO: 105)
GAGAGCAAGTACGGCCCTCCCTGCCCCCCTTGCCCTGCCCCCGAGTTCC
TGGGCGGACCCAGCGTGTTCCTGTTCCCCCCCAAGCCCAAGGACACCCT
GATGATCAGCCGGACCCCCGAGGTGACCTGTGTGGTGGTGGACGTGTCC
CAGGAGGACCCCGAGGTCCAGTTCAACTGGTACGTGGACGGCGTGGAGG
TGCACAACGCCAAGACCAAGCCCCGGGAGGAGCAGTTCAATAGCACCTA
CCGGGTGGTGTCCGTGCTGACCGTGCTGCACCAGGACTGGCTGAACGGC
AAGGAATACAAGTGTAAGGTGTCCAACAAGGGCCTGCCCAGCAGCATCG
AGAAAACCATCAGCAAGGCCAAGGGCCAGCCTCGGGAGCCCCAGGTGTA
CACCCTGCCCCCTAGCCAAGAGGAGATGACCAAGAACCAGGTGTCCCTG
ACCTGCCTGGTGAAGGGCTTCTACCCCAGCGACATCGCCGTGGAGTGGG
AGAGCAACGGCCAGCCCGAGAACAACTACAAGACCACCCCCCCTGTGCT
GGACAGCGACGGCAGCTTCTTCCTGTACAGCCGGCTGACCGTGGACAAG
AGCCGGTGGCAGGAGGGCAACGTCTTTAGCTGCTCCGTGATGCACGAGG
CCCTGCACAACCACTACACCCAGAAGAGCCTGAGCCTGTCCCTGGGCAA
GATG.

In one aspect, the hinge or spacer comprises an IgD hinge. For example, in one embodiment, the hinge or spacer comprises a hinge of the amino acid sequence

(SEQ ID NO: 106)
RWPESPKAQASSVPTAQPQAEGSLAKATTAPATTRNTGRGGEEKKKEKE
KEEQEERETKTPECPSHTQPLGVYLLTPAVQDLWLRDKATFTCFVVGSD
LKDAHLTWEVAGKVPTGGVEEGLLERHSNGSQSQHSRLTLPRSLWNAGT
SVTCTLNHPSLPPQRLMALREPAAQAPVKLSLNLLASSDPPEAASWLLC
EVSGFSPPNILLMWLEDQREVNTSGFAPARPPPQPGSTTFWAWSVLRVP
APPSPQPATYTCVVSHEDSRTLLNASRSLEVSYVTDH.

In some embodiments, the hinge or spacer comprises a hinge encoded by a nucleotide sequence of

(SEQ ID NO: 107)
AGGTGGCCCGAAAGTCCCAAGGCCCAGGCATCTAGTGTTCCTACTGCAC
AGCCCCAGGCAGAAGGCAGCCTAGCCAAAGCTACTACTGCACCTGCCAC
TACGCGCAATACTGGCCGTGGCGGGGAGGAGAAGAAAAAGGAGAAAGAG
AAAGAAGAACAGGAAGAGAGGGAGACCAAGACCCCTGAATGTCCATCCC
ATACCCAGCCGCTGGGCGTCTATCTCTTGACTCCCGCAGTACAGGACTT
GTGGCTTAGAGATAAGGCCACCTTTACATGTTTCGTCGTGGGCTCTGAC
CTGAAGGATGCCCATTTGACTTGGGAGGTTGCCGGAAAGGTACCCACAG
GGGGGGTTGAGGAAGGGTTGCTGGAGCGCCATTCCAATGGCTCTCAGAG
CCAGCACTCAAGACTCACCCTTCCGAGATCCCTGTGGAACGCCGGGACC
TCTGTCACATGTACTCTAAATCATCCTAGCCTGCCCCCACAGCGTCTGA
TGGCCCTTAGAGAGCCAGCCGCCCAGGCACCAGTTAAGCTTAGCCTGAA
TCTGCTCGCCAGTAGTGATCCCCCAGAGGCCGCCAGCTGGCTCTTATGC
GAAGTGTCCGGCTTTAGCCCGCCCAACATCTTGCTCATGTGGCTGGAGG
ACCAGCGAGAAGTGAACACCAGCGGCTTCGCTCCAGCCCGGCCCCCACC
CCAGCCGGGTTCTACCACATTCTGGGCCTGGAGTGTCTTAAGGGTCCCA
GCACCACCTAGCCCCCAGCCAGCCACATACACCTGTGTTGTGTCCCATG
AAGATAGCAGGACCCTGCTAAATGCTTCTAGGAGTCTGGAGGTTTCCTA
CGTGACTGACCATT.

In one aspect, the transmembrane domain may be recombinant, in which case it will comprise predominantly hydrophobic residues such as leucine and valine. In one aspect, a triplet of phenylalanine, tryptophan and valine can be found at each end of a recombinant transmembrane domain.

Optionally, a short oligo- or polypeptide linker, e.g., between 2 and 10 amino acids in length, may form the linkage between the transmembrane domain and the cytoplasmic region of the CAR. A glycine-serine doublet is an example of a suitable linker. For example, in one aspect, the linker comprises the amino acid sequence of GGGGSGGGGS (SEQ ID NO:108). In some embodiments, the linker is encoded by a nucleotide sequence of

(SEQ ID NO: 109)
GGTGGCGGAGGTTCTGGAGGTGGAGGTTCC.

### Cytoplasmic Domain

The cytoplasmic domain or region of the CAR includes an intracellular signaling domain. An intracellular signaling domain is generally responsible for activation of at least one of the normal effector functions of the immune cell in which the CAR has been introduced. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Examples of intracellular signaling domains for use in the CAR of the invention include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

It is known that signals generated through the TCR alone are insufficient for full activation of the T cell and that a secondary and/or costimulatory signal is also required. Thus, T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic signaling domain, e.g., a costimulatory domain).

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs.

Examples of ITAM containing primary intracellular signaling domains that are of particular use in the invention include those of TCR zeta, FcR gamma, FcR beta, CD3 gamma, CD3 delta, CD3 epsilon, CDS, CD22, CD79a, CD79b, and CD66d. In one embodiment, a CAR of the invention, e.g., a CAR selected from the group consisting of SEQ ID NO:43, SEQ ID NO:49, SEQ ID NO:55, SEQ ID NO:61, SEQ ID NO:67, SEQ ID NO:73, SEQ ID NO:79, and SEQ ID NO:85, comprises a intracellular signaling domain, e.g., a primary signaling domain, of CD3-zeta. In one embodiment, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In an embodiment, a primary signaling domain comprises one, two, three, four or more ITAM motifs.

The intracellular signaling domain of the CAR can comprise the CD3-zeta signaling domain by itself or it can be combined with any other desired intracellular signaling domain(s) useful in the context of a CAR of the invention. For example, the intracellular signaling domain of the CAR can comprise a CD3 zeta chain portion and a costimulatory signaling domain. The costimulatory signaling domain refers to a portion of the CAR comprising the intracellular domain of a costimulatory molecule. A costimulatory molecule is a cell surface molecule other than an antigen receptor or its ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like. For example, CD27 costimulation has been demonstrated to enhance expansion, effector function, and survival of human CART cells in vitro and augments human T cell persistence and antitumor activity in vivo (Song et al. Blood. 2012; 119(3):696-706).

The intracellular signaling sequences within the cytoplasmic portion of a CAR of the invention may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling sequence. In one embodiment, a glycine-serine doublet can be used as a suitable linker. In one embodiment, a single amino acid, e.g., an alanine, a glycine, can be used as a suitable linker.

In one aspect, the intracellular signaling domain is designed to comprise two or more, e.g., 2, 3, 4, 5, or more, costimulatory signaling domains. In an embodiment, the two or more, e.g., 2, 3, 4, 5, or more, costimulatory signaling domains, are separated by a linker molecule, e.g., a linker molecule described herein. In one embodiment, the intracellular signaling domain comprises two costimulatory signaling domains. In some embodiments, the linker molecule is a glycine residue. In some embodiments, the linker is an alanine residue.

In one aspect, the intracellular signaling domain is designed to comprise the signaling domain of CD3-zeta and the signaling domain of CD28. In one aspect, the intracellular signalling domain is designed to comprise the signaling domain of CD3-zeta and the signaling domain of 4-1BB. In one aspect, the signaling domain of 4-1BB is a signaling domain of SEQ ID NO: 16. In one aspect, the signaling domain of CD3-zeta is a signaling domain of SEQ ID NO: 17.

In one aspect, the intracellular signaling domain is designed to comprise the signaling domain of CD3-zeta and the signaling domain of CD27. In one aspect, the signaling domain of CD27 comprises an amino acid sequence of

(SEQ ID NO: 102)
QRRKYRSNKGESPVEPAEPCRYSCPREEEGSTIPIQEDYRKPEPACSP.

In one aspect, the signalling domain of CD27 is encoded by a nucleic acid sequence of

(SEQ ID NO: 103)
AGGAGTAAGAGGAGCAGGCTCCTGCACAGTGACTACATGAACATGACTC
CCCGCCGCCCCGGGCCCACCCGCAAGCATTACCAGCCCTATGCCCCACC
ACGCGACTTCGCAGCCTATCGCTCC.

In one aspect, the CAR-expressing cell described herein can further comprise a second CAR, e.g., a second CAR that includes a different antigen binding domain, e.g., to the same target (EGFRvIII) or a different target.

In another aspect, the present invention provides a population of CAR-expressing cells, e.g., CART cells. In some embodiments, the population of CAR-expressing cells comprises a mixture of cells expressing different CARs. For example, in one embodiment, the population of CART cells can include a first cell expressing a CAR having an anti-EGFRvIII binding domain described herein, and a second cell expressing a CAR having a different anti-EGFRvIII binding domain, e.g., an anti-EGFRvIII binding domain described herein that differs from the anti-EGFRvIII binding domain in the CAR expressed by the first cell. As another example, the population of CAR-expressing cells can include a first cell expressing a CAR that includes an anti-EGFRvIII binding domain, e.g., as described herein, and a second cell expressing a CAR that includes an antigen binding domain to a target other than EGFRvIII. In one embodiment, the population of CAR-expressing cells includes, e.g., a first cell expressing a CAR that includes a primary intracellular signaling domain, and a second cell expressing a CAR that includes a secondary signaling domain.

In another aspect, the present invention provides a population of cells wherein at least one cell in the population expresses a CAR having an anti-EGFRvIII domain described herein, and a second cell expressing another agent, e.g., an agent which enhances the activity of a CAR-expressing cell. For example, in one embodiment, the agent can be an agent which inhibits an inhibitory molecule. Inhibitory molecules, e.g., PD1, can, in some embodiments, decrease the ability of a CAR-expressing cell to mount an immune effector response. Examples of inhibitory molecules include PD1, PD-L1, CTLA4, TIM3, LAG3, VISTA, BTLA, TIGIT, LAIR1, CD160, 2B4 and TGFR beta.

### Natural Killer Cell Receptor (NKR) CARs

In an embodiment, the CAR molecule described herein comprises one or more components of a natural killer cell receptor (NKR), thereby forming an NKR-CAR. The NKR component can be a transmembrane domain, a hinge domain, or a cytoplasmic domain from any of the following natural killer cell receptors: killer cell immunoglobulin-like receptor (KIR), e.g., KIR2DL1, KIR2DL2/L3, KIR2DL4, KIR2DL5A, KIR2DL5B, KIR2DS1, KIR2DS2, KIR2DS3, KIR2DS4, DIR2DS5, KIR3DL1/S1, KIR3DL2, KIR3DL3, KIR2DP1, and KIR3DP1; natural cytotoxicity receptor (NCR), e.g., NKp30, NKp44, NKp46; signaling lymphocyte activation molecule (SLAM) family of immune cell receptors, e.g., CD48, CD229, 2B4, CD84, NTB-A, CRACC, BLAME, and CD2F-10; Fc receptor (FcR), e.g., CD16, and CD64; and Ly49 receptors, e.g., LY49A, LY49C. The NKR-CAR molecules described herein may interact with an adaptor molecule or intracellular signaling domain, e.g., DAP12. Exemplary configurations and sequences of CAR molecules comprising NKR components are described in International Publication No. WO2014/145252, the contents of which are hereby incorporated by reference.

### Split CAR

In some embodiments, the CAR-expressing cell described herein, uses a split CAR. The split CAR approach is described in more detail in publications WO2014/055442 and WO2014/055657, incorporated herein by reference. Briefly, a split CAR system comprises a cell expressing a first CAR having a first antigen binding domain and a costimulatory domain (e.g., 4-1BB), and the cell also expresses a second CAR having a second antigen binding domain and an intracellular signaling domain (e.g., CD3 zeta). When the cell encounters the first antigen, the costimulatory domain is activated, and the cell proliferates. When the cell encounters the second antigen, the intracellular signaling domain is activated and cell-killing activity begins. Thus, the CAR-expressing cell is only fully activated in the presence of both antigens. In embodiments the first antigen binding domain recognizes the tumor antigen described herein, e.g., comprises an antigen binding domain described herein, and the second antigen binding domain recognizes a second antigen, e.g., a second tumor antigen described herein.

Co-Expression of CAR with Other Molecules or Agents

Co-Expression of a Second CAR

In one aspect, the CAR-expressing cell described herein can further comprise a second CAR, e.g., a second CAR that includes a different antigen binding domain, e.g., to the same target (EGFRvIII) or a different target (e.g., a target other than EGFRvIII). In one embodiment, the CAR-expressing cell comprises a first CAR that targets a first antigen and includes an intracellular signaling domain having a costimulatory signaling domain but not a primary signaling domain, and a second CAR that targets a second, different, antigen and includes an intracellular signaling domain having a primary signaling domain but not a costimulatory signaling domain. Placement of a costimulatory signaling domain, e.g., 4-1BB, CD28, CD27, OX-40 or ICOS, onto the first CAR, and the primary signaling domain, e.g., CD3 zeta, on the second CAR can limit the CAR activity to cells where both targets are expressed. In one embodiment, the CAR expressing cell comprises a first EGFRvIII CAR that includes an EGFRvIII binding domain, a transmembrane domain and a costimulatory domain and a second CAR that targets an antigen other than EGFRvIII and includes an antigen binding domain, a transmembrane domain and a primary signaling domain. In another embodiment, the CAR expressing cell comprises a first EGFRvIII CAR that includes an EGFRvIII binding domain, a transmembrane domain and a primary signaling domain and a second CAR that targets an antigen other than EGFRvIII and includes an antigen binding domain to the antigen, a transmembrane domain and a costimulatory signaling domain.

In one embodiment, the CAR-expressing cell comprises an EGFRvIII CAR described herein and an inhibitory CAR. In one embodiment, the inhibitory CAR comprises an antigen binding domain that binds an antigen found on normal cells but not cancer cells, e.g., normal cells that also express EGFRvIII. In one embodiment, the inhibitory CAR comprises the antigen binding domain, a transmembrane domain and an intracellular domain of an inhibitory molecule. For example, the intracellular domain of the inhibitory CAR can be an intracellular domain of PD1, PD-L1, CTLA4, TIM3, LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, or TGF beta.

In one embodiment, when the CAR-expressing cell comprises two or more different CARs, the antigen binding domains of the different CARs can be such that the antigen binding domains do not interact with one another. For example, a cell expressing a first and second CAR can have an antigen binding domain of the first CAR, e.g., as a fragment, e.g., an scFv, that does not form an association with the antigen binding domain of the second CAR, e.g., the antigen binding domain of the second CAR is a VHH.

In some embodiments, the antigen binding domain comprises a single domain antigen binding (SDAB) molecules include molecules whose complementary determining regions are part of a single domain polypeptide. Examples include, but are not limited to, heavy chain variable domains, binding molecules naturally devoid of light chains, single domains derived from conventional 4-chain antibodies, engineered domains and single domain scaffolds other than those derived from antibodies. SDAB molecules may be any of the art, or any future single domain molecules. SDAB molecules may be derived from any species including, but not limited to mouse, human, camel, llama, lamprey, fish, shark, goat, rabbit, and bovine. This term also includes naturally occurring single domain antibody molecules from species other than Camelidae and sharks.

In one aspect, an SDAB molecule can be derived from a variable region of the immunoglobulin found in fish, such as, for example, that which is derived from the immunoglobulin isotype known as Novel Antigen Receptor (NAR) found in the serum of shark. Methods of producing single domain molecules derived from a variable region of NAR (“IgNARs”) are described in WO 03/014161 and Streltsov (2005) Protein Sci. 14:2901-2909.

According to another aspect, an SDAB molecule is a naturally occurring single domain antigen binding molecule known as heavy chain devoid of light chains. Such single domain molecules are disclosed in WO 9404678 and Hamers-Casterman, C. et al. (1993) Nature 363:446-448, for example. For clarity reasons, this variable domain derived from a heavy chain molecule naturally devoid of light chain is known herein as a VHH or nanobody to distinguish it from the conventional VH of four chain immunoglobulins. Such a VHH molecule can be derived from Camelidae species, for example in camel, llama, dromedary, alpaca and guanaco. Other species besides Camelidae may produce heavy chain molecules naturally devoid of light chain; such VHHs are within the scope of the invention.

The SDAB molecules can be recombinant, CDR-grafted, humanized, camelized, deimmunized and/or in vitro generated (e.g., selected by phage display).

It has also been discovered, that cells having a plurality of chimeric membrane embedded receptors comprising an antigen binding domain that interactions between the antigen binding domain of the receptors can be undesirable, e.g., because it inhibits the ability of one or more of the antigen binding domains to bind its cognate antigen. Accordingly, disclosed herein are cells having a first and a second non-naturally occurring chimeric membrane embedded receptor comprising antigen binding domains that minimize such interactions. Also disclosed herein are nucleic acids encoding a first and a second non-naturally occurring chimeric membrane embedded receptor comprising an antigen binding domains that minimize such interactions, as well as methods of making and using such cells and nucleic acids. In an embodiment the antigen binding domain of one of the first and the second non-naturally occurring chimeric membrane embedded receptor, comprises an scFv, and the other comprises a single VH domain, e.g., a camelid, shark, or lamprey single VH domain, or a single VH domain derived from a human or mouse sequence.

In some embodiments, the claimed invention comprises a first and second CAR, wherein the antigen binding domain of one of the first and the second CAR does not comprise a variable light domain and a variable heavy domain. In some embodiments, the antigen binding domain of one of the first and the second CAR is an scFv, and the other is not an scFv. In some embodiments, the antigen binding domain of one of the first and the second CAR comprises a single VH domain, e.g., a camelid, shark, or lamprey single VH domain, or a single VH domain derived from a human or mouse sequence. In some embodiments, the antigen binding domain of one of the first and the second CAR comprises a nanobody. In some embodiments, the antigen binding domain of one of the first and the second CAR comprises a camelid VHH domain.

In some embodiments, the antigen binding domain of one of the first and the second CAR comprises an scFv, and the other comprises a single VH domain, e.g., a camelid, shark, or lamprey single VH domain, or a single VH domain derived from a human or mouse sequence. In some embodiments, the antigen binding domain of one of the first and the second CAR comprises an scFv, and the other comprises a nanobody. In some embodiments, the antigen binding domain of one of the first and the second CAR comprises comprises an scFv, and the other comprises a camelid VHH domain.

In some embodiments, when present on the surface of a cell, binding of the antigen binding domain of the first CAR to its cognate antigen is not substantially reduced by the presence of the second CAR. In some embodiments, binding of the antigen binding domain of the first CAR to its cognate antigen in the presence of the second CAR is 85%, 90%, 95%, 96%, 97%, 98% or 99% of binding of the antigen binding domain of the first CAR to its cognate antigen in the absence of the second CAR.

In some embodiments, when present on the surface of a cell, the antigen binding domains of the first and the second CAR, associate with one another less than if both were scFv antigen binding domains. In some embodiments, the antigen binding domains of the first and the second CAR, associate with one another 85%, 90%, 95%, 96%, 97%, 98% or 99% less than if both were scFv antigen binding domains.

Co-Expression of an Agent that Enhances CAR Activity

In another aspect, the CAR-expressing cell described herein can further express another agent, e.g., an agent that enhances the activity or fitness of a CAR-expressing cell.

For example, in one embodiment, the agent can be an agent which inhibits a molecule that modulates or regulates, e.g., inhibits, T cell function. In some embodiments, the molecule that modulates or regulates T cell function is an inhibitory molecule. Inhibitory molecules, e.g., PD-1, can, in some embodiments, decrease the ability of a CAR-expressing cell to mount an immune effector response. Examples of inhibitory molecules include PD-1, PD-L1, CTLA4, TIM3, LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, or TGF beta.

In embodiments, an agent, e.g., an inhibitory nucleic acid, e.g., a dsRNA, e.g., an siRNA or shRNA; or e.g., an inhibitory protein or system, e.g., a clustered regularly interspaced short palindromic repeats (CRISPR), a transcription-activator like effector nuclease (TALEN), or a zinc finger endonuclease (ZFN), e.g., as described herein, can be used to inhibit expression of a molecule that modulates or regulates, e.g., inhibits, T-cell function in the CAR-expressing cell. In an embodiment the agent is an shRNA, e.g., an shRNA described herein. In an embodiment, the agent that modulates or regulates, e.g., inhibits, T-cell function is inhibited within a CAR-expressing cell. For example, a dsRNA molecule that inhibits expression of a molecule that modulates or regulates, e.g., inhibits, T-cell function is linked to the nucleic acid that encodes a component, e.g., all of the components, of the CAR.

In one embodiment, the agent that inhibits an inhibitory molecule comprises a first polypeptide, e.g., an inhibitory molecule, associated with a second polypeptide that provides a positive signal to the cell, e.g., an intracellular signaling domain described herein. In one embodiment, the agent comprises a first polypeptide, e.g., of an inhibitory molecule such as PD-1, PD-L1, CTLA4, TIM3, LAGS, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, or TGF beta, or a fragment of any of these (e.g., at least a portion of an extracellular domain of any of these), and a second polypeptide which is an intracellular signaling domain described herein (e.g., comprising a costimulatory domain (e.g., 4-1BB, CD27 or CD28, e.g., as described herein) and/or a primary signaling domain (e.g., a CD3 zeta signaling domain described herein). In one embodiment, the agent comprises a first polypeptide of PD-1 or a fragment thereof (e.g., at least a portion of an extracellular domain of PD-1), and a second polypeptide of an intracellular signaling domain described herein (e.g., a CD28 signaling domain described herein and/or a CD3 zeta signaling domain described herein). PD-1 is an inhibitory member of the CD28 family of receptors that also includes CD28, CTLA-4, ICOS, and BTLA. PD-1 is expressed on activated B cells, T cells and myeloid cells (Agata et al. 1996 Int. Immunol 8:765-75). Two ligands for PD-1, PD-L₁ and PD-L2 have been shown to downregulate T cell activation upon binding to PD-1 (Freeman et a. 2000 J Exp Med 192:1027-34; Latchman et al. 2001 Nat Immunol 2:261-8; Carter et al. 2002 Eur J Immunol 32:634-43). PD-L1 is abundant in human cancers (Dong et al. 2003 J Mol Med 81:281-7; Blank et al. 2005 Cancer Immunol. Immunother 54:307-314; Konishi et al. 2004 Clin Cancer Res 10:5094). Immune suppression can be reversed by inhibiting the local interaction of PD-1 with PD-L1.

In one embodiment, the agent comprising the extracellular domain (ECD) of an inhibitory molecule, e.g., Programmed Death 1 (PD-1), can be fused to a transmembrane domain and intracellular signaling domains such as 4-1BB and CD3 zeta (also referred to herein as a PD1 CAR). In one embodiment, the PD1 CAR, when used in combinations with an EGFRvIII CAR described herein, improves the persistence of the T cell. In one embodiment, the CAR is a PD1 CAR comprising the extracellular domain of PD-1 indicated as underlined in SEQ ID NO: 130 and a signal sequence at amino acids 1-21 of SEQ ID NO: 130. In one embodiment, the PD1 CAR comprises the amino acid sequence of SEQ ID NO: 130.

In one embodiment, the PD1 CAR without the N-terminal signal sequence comprises the amino acid sequence provided of SEQ ID NO: 128.

In one embodiment, the agent comprises a nucleic acid sequence encoding the PD1 CAR with the N-terminal signal sequence, e.g., the PD1 CAR described herein. In one embodiment, the nucleic acid sequence for the PD1 CAR, with the PD1 ECD underlined in SEQ ID NO: 129:

| | | |
|---|---|---|
| SEQ | PD1 CAR (aa) | Pgwfldspdrpwnpptfspallvvtegdnatftcsfsntsesfvlnwyrmspsnqtdklaafpedrsqpgqdc |
| ID | (PD1 ECD | rfrvtqlpngrdfhmsvvrarrndsgtylcgaislapkaqikeslraelrvterraevptahpspsprpagqfq |
| NO: | underlined) | tlvtttpaprpptpaptiasqplslrpeacrpaaggavhtrgldfacdiyiwaplagtcgvlllslvitlyckr |
| 128 | | grkkllyifkqpfmrpvqttqeedgcscrfpeeeeggcelrvkfsrsadapaykqgqnqlynelnlgrreeydv |
| | | ldkrrgrdpemggkprrknpqeglynelqkdkmaeayseigmkgerrrgkghdglyqglstatkdtydalhmqa |
| | | lppr |
| SEQ | PD-1 CAR | atggccctccctgtcactgccctgcttctccccctcgcactcctgctccacgccgctagaccacccggatggtt |
| ID | (na) | tctggactctccggatcgcccgtggaatcccccaaccactcaccggcactcaggttgtgactgagggcgataat |
| NO: | (PD1 ECD | gcgaccttcacgtgctcgttctccaacacctccgaatcattcgtgctgaactggtaccgcatgagcccgtcaaa |
| 129 | underlined) | ccagaccgacaagctcgccgcgtttccggaagatcggtcgcaaccgggacaggattgtcggttccgcgtgactc |
| | | aactgccgaatggcagagacttccacatgagcgtggtccgcgctaggcgaaacgactccgggacctacctgtgc |
| | | ggagccatctcgctggcgcctaaggcccaaatcaaagagagcttgagggccgaactgagagtgaccgagcgcag |
| | | agctgaggtgccaactgcacatccatccccatcgcctcggcctgcggggcagtttcagaccctggtcacgacca |
| | | ctccggcgccgcgcccaccgactccggccccaactatcgcgagccagcccctgtcgctgaggccggaagcatgc |
| | | cgccctgccgccggaggtgctgtgcatacccggggattggacttcgcatgcgacatctacatttgggctcctct |
| | | cgccggaacttgtggcgtgctccttctgtccctggtcatcaccctgtactgcaagcggggtcggaaaaagcttc |
| | | tgtacattttcaagcagcccttcatgaggcccgtgcaaaccacccaggaggaggacggttgctcctgccggttc |
| | | cccgaagaggaagaaggaggttgcgagctgcgcgtgaagttctcccggagcgccgacgcccccgcctataagca |
| | | gggccagaaccagctgtacaacgaactgaacctgggacggcgggaagagtacgatgtgctggacaagcggcgcg |
| | | gccgggaccccgaaatgggcgggaagcctagaagaaagaaccctcaggaaggcctgtataacgagctgcagaag |
| | | gacaagatggccgaggcctactccgaaattgggatgaagggagagcggcggaggggaaaggggcacgacggcct |
| | | gtaccaaggactgtccaccgccaccaaggacacatacgatgccctgcacatgcaggcccttccccctcgc |
| SEQ | PD-1 CAR | Malpvtalllplalllhaarppgwfldspdrpwnpptfspallvvtegdnatftcsfsntsesfvlnwyrmsps |
| ID | (aa) | nqtdklaafpedrsqpgqdcrfrvtqlpngrdfhmsvvrarrndsgtylcgaislapkaqikeslraelrvter |
| NO: | with signal | raevptahpspsprpagqfqtlvtttpaprpptpaptiasqplslrpeacrpaaggavhtrgldfacdiyiwap |
| 130 | (PD1 ECD | lagtcgvlllslvitlyclagrkkllyifkqpfmrpvqttqeedgcscrfpeeeeggcelrvkfsrsadapayk |
| | underlined) | qgqnqlynelnlgrreeydvldkrrgrdpemggkprrknpqeglynelqkdkmaeayseigmkgeragkghdgl |
| | | yqglstatkdtydalhmqalppr |

In another example, in one embodiment, the agent which enhances the activity of a CAR-expressing cell can be a costimulatory molecule or costimulatory molecule ligand. Examples of costimulatory molecules include MHC class I molecule, BTLA and a Toll ligand receptor, as well as OX40, CD27, CD28, CDS, ICAM-1, LFA-1 (CD11a/CD18), ICOS (CD278), and 4-1BB (CD137). Further examples of such costimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83., e.g., as described herein. Examples of costimulatory molecule ligands include CD80, CD86, CD40L, ICOSL, CD70, OX40L, 4-1BBL, GITRL, and LIGHT. In embodiments, the costimulatory molecule ligand is a ligand for a costimulatory molecule different from the costimulatory molecule domain of the CAR. In embodiments, the costimulatory molecule ligand is a ligand for a costimulatory molecule that is the same as the costimulatory molecule domain of the CAR. In an embodiment, the costimulatory molecule ligand is 4-1BBL. In an embodiment, the costimulatory ligand is CD80 or CD86. In an embodiment, the costimulatory molecule ligand is CD70. In embodiments, a CAR-expressing immune effector cell described herein can be further engineered to express one or more additional costimulatory molecules or costimulatory molecule ligands.

Co-Expression of CAR with a Chemokine Receptor

In embodiments, the CAR-expressing cell described herein, e.g., EGFRvIII CAR-expressing cell, further comprises a chemokine receptor molecule. Transgenic expression of chemokine receptors CCR2b or CXCR2 in T cells enhances trafficking to CCL2- or CXCL1-secreting solid tumors including melanoma and neuroblastoma (Craddock et al., J Immunother. 2010 October; 33(8):780-8 and Kershaw et al., Hum Gene Ther. 2002 Nov. 1; 13(16):1971-80). Thus, without wishing to be bound by theory, it is believed that chemokine receptors expressed in CAR-expressing cells that recognize chemokines secreted by tumors, e.g., solid tumors, can improve homing of the CAR-expressing cell to the tumor, facilitate the infiltration of the CAR-expressing cell to the tumor, and enhances antitumor efficacy of the CAR-expressing cell. The chemokine receptor molecule can comprise a naturally occurring or recombinant chemokine receptor or a chemokine-binding fragment thereof. A chemokine receptor molecule suitable for expression in a CAR-expressing cell (e.g., CAR-Tx) described herein include a CXC chemokine receptor (e.g., CXCR1, CXCR2, CXCR3, CXCR4, CXCR5, CXCR6, or CXCR7), a CC chemokine receptor (e.g., CCR1, CCR2, CCR3, CCR4, CCR5, CCR6, CCR7, CCR8, CCR9, CCR10, or CCR11), a CX3C chemokine receptor (e.g., CX3CR1), a XC chemokine receptor (e.g., XCR1), or a chemokine-binding fragment thereof. In one embodiment, the chemokine receptor molecule to be expressed with a CAR described herein is selected based on the chemokine(s) secreted by the tumor. In one embodiment, the CAR-expressing cell described herein further comprises, e.g., expresses, a CCR2b receptor or a CXCR2 receptor. In an embodiment, the CAR described herein and the chemokine receptor molecule are on the same vector or are on two different vectors. In embodiments where the CAR described herein and the chemokine receptor molecule are on the same vector, the CAR and the chemokine receptor molecule are each under control of two different promoters or are under the control of the same promoter.

### Nucleic Acid Constructs Encoding a CAR

The present invention provides nucleic acid molecules encoding one or more CAR constructs described herein. In one aspect, the nucleic acid molecule is provided as a messenger RNA transcript. In one aspect, the nucleic acid molecule is provided as a DNA construct.

Accordingly, in one aspect, the invention pertains to an isolated nucleic acid molecule encoding a chimeric antigen receptor (CAR), wherein the CAR comprises a anti-EGFRvIII binding domain (e.g., a humanized anti-EGFRvIII binding domain), a transmembrane domain, and an intracellular signaling domain comprising a stimulatory domain, e.g., a costimulatory signaling domain and/or a primary signaling domain, e.g., zeta chain. In one embodiment, the anti-EGFRvIII binding domain is an anti-EGFRvIII binding domain described herein, e.g., an anti-EGFRvIII binding domain which comprises a sequence selected from a group consisting of SEQ ID NO:38, SEQ ID NO:44, SEQ ID NO:50, SEQ ID NO:56, SEQ ID NO:62, SEQ ID NO:68, SEQ ID NO:74, and SEQ ID NO:80, or a sequence with 95-99% identify thereof. In one embodiment, the isolated nucleic acid molecule further comprises a sequence encoding a costimulatory domain. In one embodiment, the costimulatory domain is a functional signaling domain of a protein selected from the group consisting of OX40, CD27, CD28, CDS, ICAM-1, LFA-1 (CD11a/CD18), ICOS (CD278), and 4-1BB (CD137). In one embodiment, the costimulatory domain comprises a sequence of SEQ ID NO:16, or a sequence with 95-99% identity thereof. In one embodiment, the transmembrane domain is transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 and CD154. In one embodiment, the transmembrane domain comprises a sequence of SEQ ID NO: 15, or a sequence with 95-99% identity thereof. In one embodiment, the intracellular signaling domain comprises a functional signaling domain of 4-1BB and a functional signaling domain of CD3 zeta. In one embodiment, the intracellular signaling domain comprises the sequence of SEQ ID NO: 16 or SEQ ID NO:102, or a sequence with 95-99% identity thereof, and the sequence of SEQ ID NO: 17 or SEQ ID NO:99, or a sequence with 95-99% identity thereof, wherein the sequences comprising the intracellular signaling domain are expressed in the same frame and as a single polypeptide chain. In one embodiment, the anti-EGFRvIII binding domain is connected to the transmembrane domain by a hinge region, e.g., a hinge described herein. In one embodiment, the hinge region comprises SEQ ID NO:14 or SEQ ID NO:104 or SEQ ID NO:106 or SEQ ID NO:108, or a sequence with 95-99% identity thereof.

In another aspect, the invention pertains to an isolated nucleic acid molecule encoding a CAR construct comprising a leader sequence of SEQ ID NO: 13, a scFv domain having a sequence selected from the group consisting of SEQ ID NO:38, SEQ ID NO:44, SEQ ID NO:50, SEQ ID NO:56, SEQ ID NO:62, SEQ ID NO:68, SEQ ID NO:74, SEQ ID NO:80, and SEQ ID NO:86 (or a sequence with 95-99% identify thereof), a hinge region of SEQ ID NO:14 or SEQ ID NO:104 or SEQ ID NO:106 or SEQ ID NO:108 (or a sequence with 95-99% identity thereof), a transmembrane domain having a sequence of SEQ ID NO: 15 (or a sequence with 95-99% identity thereof), a 4-1BB costimulatory domain having a sequence of SEQ ID NO:16 or a CD27 costimulatory domain having a sequence of SEQ ID NO:102 (or a sequence with 95-99% identity thereof), and a CD3 zeta stimulatory domain having a sequence of SEQ ID NO:17 or SEQ ID NO:99 (or a sequence with 95-99% identity thereof).

In another aspect, the invention pertains to an isolated polypeptide molecule encoded by the nucleic acid molecule. In one embodiment, the isolated polypeptide molecule comprises a sequence selected from the group consisting of SEQ ID NO:43, SEQ ID NO:49, SEQ ID NO:55, SEQ ID NO:61, SEQ ID NO:67, SEQ ID NO:73, SEQ ID NO:79, SEQ ID NO:85, and SEQ ID NO:90 or a sequence with 95-99% identify thereof. In one embodiment, the isolated polypeptide comprises a sequence of SEQ ID NO:73, or a sequence with 95-99% identify thereof. In one embodiment, the isolated polypeptide comprises a sequence of SEQ ID NO:79, or a sequence with 95-99% identify thereof.

In another aspect, the invention pertains to a nucleic acid molecule encoding a chimeric antigen receptor (CAR) molecule that comprises an anti-EGFRvIII binding domain, a transmembrane domain, and an intracellular signaling domain comprising a stimulatory domain, and wherein said anti-EGFRvIII binding domain comprises a sequence selected from the group consisting of SEQ ID NO:38, SEQ ID NO:44, SEQ ID NO:50, SEQ ID NO:56, SEQ ID NO:62, SEQ ID NO:68, SEQ ID NO:74, SEQ ID NO:80, and SEQ ID NO:86, or a sequence with 95-99% identify thereof.

In one embodiment, the encoded CAR molecule further comprises a sequence encoding a costimulatory domain. In one embodiment, the costimulatory domain is a functional signaling domain of a protein selected from the group consisting of OX40, CD27, CD28, CDS, ICAM-1, LFA-1 (CD11a/CD18) and 4-1BB (CD137). In one embodiment, the costimulatory domain comprises a sequence of SEQ ID NO:16. In one embodiment, the transmembrane domain is a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 and CD154. In one embodiment, the transmembrane domain comprises a sequence of SEQ ID NO:15. In one embodiment, the intracellular signaling domain comprises a functional signaling domain of 4-1BB and a functional signaling domain of zeta. In one embodiment, the intracellular signaling domain comprises the sequence of SEQ ID NO: 16 and the sequence of SEQ ID NO: 17, wherein the sequences comprising the intracellular signaling domain are expressed in the same frame and as a single polypeptide chain. In one embodiment, the anti-EGFRvIII binding domain is connected to the transmembrane domain by a hinge region. In one embodiment, the hinge region comprises SEQ ID NO:14. In one embodiment, the hinge region comprises SEQ ID NO:104 or SEQ ID NO:106 or SEQ ID NO:108

In another aspect, the invention pertains to an encoded CAR molecule comprising a leader sequence of SEQ ID NO: 13, a scFv domain having a sequence selected from the group consisting of SEQ ID NO:38, SEQ ID NO:44, SEQ ID NO:50, SEQ ID NO:56, SEQ ID NO:62, SEQ ID NO:68, SEQ ID NO:74, SEQ ID NO:80, and SEQ ID NO:86, or a sequence with 95-99% identify thereof, a hinge region of SEQ ID NO:14 or SEQ ID NO:104 or SEQ ID NO:106 or SEQ ID NO:108, a transmembrane domain having a sequence of SEQ ID NO: 15, a 4-1BB costimulatory domain having a sequence of SEQ ID NO:16 or a CD27 costimulatory domain having a sequence of SEQ ID NO:102, and a CD3 zeta stimulatory domain having a sequence of SEQ ID NO:17 or SEQ ID NO:99. In one embodiment, the encoded CAR molecule comprises a sequence selected from a group consisting of SEQ ID NO:43, SEQ ID NO:49, SEQ ID NO:55, SEQ ID NO:61, SEQ ID NO:67, SEQ ID NO:73, SEQ ID NO:79, SEQ ID NO:85, and SEQ ID NO:90, or a sequence with 95-99% identify thereof. In one embodiment, the encoded CAR molecule comprises a sequence of SEQ ID NO:73, or a sequence with 95-99% identify thereof. In one embodiment, the isolated CAR molecule comprises a sequence of SEQ ID NO:79, or a sequence with 95-99% identify thereof.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The present invention also provides vectors in which a DNA of the present invention is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

In brief summary, the expression of natural or synthetic nucleic acids encoding CARs is typically achieved by operably linking a nucleic acid encoding the CAR polypeptide or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The expression constructs of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a nanoparticle, e.g., a liposome or other suitable sub-micron sized delivery system. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

The present invention further provides a vector comprising a CAR encoding nucleic acid molecule. In one aspect, a CAR vector can be directly transduced into a cell, e.g., a T cell. In one aspect, the vector is a cloning or expression vector, e.g., a vector including, but not limited to, one or more plasmids (e.g., expression plasmids, cloning vectors, minicircles, minivectors, double minute chromosomes), retroviral and lentiviral vector constructs. In one aspect, the vector is capable of expressing the CAR construct in mammalian T cells. In one aspect, the mammalian T cell is a human T cell.

### RNA Transfection

Disclosed herein are methods for producing an in vitro transcribed RNA CAR. The present invention also includes a CAR encoding RNA construct that can be directly transfected into a cell. A method for generating mRNA for use in transfection can involve in vitro transcription (IVT) of a template with specially designed primers, followed by polyA addition, to produce a construct containing 3′ and 5′ untranslated sequence (“UTR”), a 5′ cap and/or Internal Ribosome Entry Site (IRES), the nucleic acid to be expressed, and a polyA tail, typically 50-2000 bases in length (SEQ ID NO: 116). RNA so produced can efficiently transfect different kinds of cells. In one aspect, the template includes sequences for the CAR.

In one aspect the EGFRvIII CAR is encoded by a messenger RNA (mRNA). In one aspect the mRNA encoding the EGFRvIII CAR is introduced into a T cell for production of a CART cell.

In one embodiment, the in vitro transcribed RNA CAR can be introduced to a cell as a form of transient transfection. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired temple for in vitro transcription is a CAR of the present invention. For example, the template for the RNA CAR comprises an extracellular region comprising a single chain variable domain of an anti-tumor antibody; a hinge region, a transmembrane domain (e.g., a transmembrane domain of CD8a); and a cytoplasmic region that includes an intracellular signaling domain, e.g., comprising the signaling domain of CD3-zeta and the signaling domain of 4-1BB.

In one embodiment, the DNA to be used for PCR contains an open reading frame. The DNA can be from a naturally occurring DNA sequence from the genome of an organism. In one embodiment, the nucleic acid can include some or all of the 5′ and/or 3′ untranslated regions (UTRs). The nucleic acid can include exons and introns. In one embodiment, the DNA to be used for PCR is a human nucleic acid sequence. In another embodiment, the DNA to be used for PCR is a human nucleic acid sequence including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

PCR is used to generate a template for in vitro transcription of mRNA which is used for transfection. Methods for performing PCR are well known in the art. Primers for use in PCR are designed to have regions that are substantially complementary to regions of the DNA to be used as a template for the PCR. “Substantially complementary,” as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a nucleic acid that is normally transcribed in cells (the open reading frame), including 5′ and 3′ UTRs. The primers can also be designed to amplify a portion of a nucleic acid that encodes a particular domain of interest. In one embodiment, the primers are designed to amplify the coding region of a human cDNA, including all or portions of the 5′ and 3′ UTRs. Primers useful for PCR can be generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Any DNA polymerase useful for PCR can be used in the methods disclosed herein. The reagents and polymerase are commercially available from a number of sources.

Chemical structures with the ability to promote stability and/or translation efficiency may also be used. The RNA preferably has 5′ and 3′ UTRs. In one embodiment, the 5′ UTR is between one and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the nucleic acid of interest. Alternatively, UTR sequences that are not endogenous to the nucleic acid of interest can be added by incorporating the UTR sequences into the forward and reverse primers or by any other modifications of the template. The use of UTR sequences that are not endogenous to the nucleic acid of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous nucleic acid. Alternatively, when a 5′ UTR that is not endogenous to the nucleic acid of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be 5′UTR of an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one preferred embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In a preferred embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The polyA/T segment of the transcriptional DNA template can be produced during PCR by using a reverse primer containing a polyT tail, such as 100T tail (SEQ ID NO: 117) (size can be 50-5000 T (SEQ ID NO: 118)), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines (SEQ ID NO: 119).

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides (SEQ ID NO: 120) results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps on also provide stability to RNA molecules. In a preferred embodiment, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

RNA can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001

### Non-Viral Delivery Methods

In some aspects, non-viral methods can be used to deliver a nucleic acid encoding a CAR described herein into a cell or tissue or a subject. In some embodiments, the non-viral method includes the use of a transposon (also called a transposable element). In some embodiments, a transposon is a piece of DNA that can insert itself at a location in a genome, for example, a piece of DNA that is capable of self-replicating and inserting its copy into a genome, or a piece of DNA that can be spliced out of a longer nucleic acid and inserted into another place in a genome.

Additional and exemplary transposons and non-viral delivery methods are described on pages 196-198 of International Application WO 2016/164731, filed Apr. 8, 2016, which is incorporated by reference in its entirety.

### Sources of T Cells

Prior to expansion and genetic modification, e.g., to express a CAR described herein, a source of cells, e.g., T cell or NK cells, can be obtained from a subject. The term “subject” is intended to include living organisms in which an immune response can be elicited (e.g., mammals). Examples of subjects include humans, dogs, cats, mice, rats, and transgenic species thereof.

In embodiments, immune effector cells (e.g., a population of immune effector cells), e.g., T cells, are derived from (e.g., differentiated from) a stem cell, e.g., an embryonic stem cell or a pluripotent stem cell, e.g., an induced pluripotent stem cell (iPSC). In embodiments, the cells are autologous or allogeneic. In embodiments wherein the cells are allogeneic, the cells, e.g., derived from stem cells (e.g., iPSCs), are modified to reduce their alloreactivity. For example, the cells can be modified to reduce alloreactivity, e.g., by modifying (e.g., disrupting) their T cell receptor. In embodiments, a site specific nuclease can be used to disrupt the T cell receptor, e.g., after T-cell differentiation. In other examples, cells, e.g., T cells derived from iPSCs, can be generated from virus-specific T cells, which are less likely to cause graft-versus-host disease because of their recognition of a pathogen-derived antigen. In yet other examples, alloreactivity can be reduced, e.g., minimized, by generating iPSCs from common HLA haplotypes such that they are histocompatible with matched, unrelated recipient subjects. In yet other examples, alloreactivity can be reduced, e.g., minimized, by repressing HLA expression through genetic modification. For example, T cells derived from iPSCs can be processed as described in, e.g., Themeli et al. Nat. Biotechnol. 31.10(2013):928-35, incorporated herein by reference. In some examples, immune effector cells, e.g., T cells, derived from stem cells, can be processed/generated using methods described in WO2014/165707, incorporated herein by reference. Additional embodiments pertaining to allogeneic cells are described herein, e.g., in the “Allogeneic CAR Immune Effector Cells” section herein.

T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain aspects of the present disclosure, any number of T cell lines available in the art, may be used. In certain aspects of the present disclosure, T cells can be obtained from a unit of blood collected from a subject using any number of techniques known to the skilled artisan, such as Ficoll™ separation. In one preferred aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and to place the cells in an appropriate buffer or media for subsequent processing steps. In one aspect of the invention, the cells are washed with phosphate buffered saline (PBS). In an alternative aspect, the wash solution lacks calcium and may lack magnesium or may lack many if not all divalent cations. Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

It is recognized that the methods of the application can utilize culture media conditions comprising 5% or less, for example 2%, human AB serum, and employ known culture media conditions and compositions, for example those described in Smith et al., “Ex vivo expansion of human T cells for adoptive immunotherapy using the novel Xeno-free CTS Immune Cell Serum Replacement” Clinical & Translational Immunology (2015) 4, e31; doi:10.1038/cti.2014.31.

In one aspect, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3+, CD28+, CD4+, CD8+, CD45RA+, and CD45RO+ T cells, can be further isolated by positive or negative selection techniques. For example, in one aspect, T cells are isolated by incubation with anti-CD3/anti-CD28 (e.g., 3×28)-conjugated beads, such as DYNABEADS® M-450 CD3/CD28 T, for a time period sufficient for positive selection of the desired T cells. In one aspect, the time period is about 30 minutes. In a further aspect, the time period ranges from 30 minutes to 36 hours or longer and all integer values there between. In a further aspect, the time period is at least 1, 2, 3, 4, 5, or 6 hours. In yet another preferred aspect, the time period is 10 to 24 hours. In one aspect, the incubation time period is 24 hours. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In certain aspects, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In certain aspects, it may be desirable to enrich for or positively select for regulatory T cells which typically express CD4+, CD25+, CD62Lhi, GITR+, and FoxP3+. Alternatively, in certain aspects, T regulatory cells are depleted by anti-C25 conjugated beads or other similar method of selection.

The methods described herein can include, e.g., selection of a specific subpopulation of immune effector cells, e.g., T cells, that are a T regulatory cell-depleted population, CD25+ depleted cells, using, e.g., a negative selection technique, e.g., described herein. Preferably, the population of T regulatory depleted cells contains less than 30%, 25%, 20%, 15%, 10%, 5%, 4%, 3%, 2%, 1% of CD25+ cells.

In one embodiment, T regulatory cells, e.g., CD25+ T cells, are removed from the population using an anti-CD25 antibody, or fragment thereof, or a CD25-binding ligand, IL-2. In one embodiment, the anti-CD25 antibody, or fragment thereof, or CD25-binding ligand is conjugated to a substrate, e.g., a bead, or is otherwise coated on a substrate, e.g., a bead. In one embodiment, the anti-CD25 antibody, or fragment thereof, is conjugated to a substrate as described herein.

In one embodiment, the T regulatory cells, e.g., CD25+ T cells, are removed from the population using CD25 depletion reagent from Miltenyi™. In one embodiment, the ratio of cells to CD25 depletion reagent is 1e7 cells to 20 uL, or 1e7 cells to 15 uL, or 1e7 cells to 10 uL, or 1e7 cells to 5 uL, or 1e7 cells to 2.5 uL, or 1e7 cells to 1.25 uL. In one embodiment, e.g., for T regulatory cells, e.g., CD25+ depletion, greater than 500 million cells/ml is used. In a further aspect, a concentration of cells of 600, 700, 800, or 900 million cells/ml is used.

In one embodiment, the population of immune effector cells to be depleted includes about 6×10⁹ CD25+ T cells. In other aspects, the population of immune effector cells to be depleted include about 1×10⁹ to 1×10¹⁰ CD25+ T cell, and any integer value in between. In one embodiment, the resulting population T regulatory depleted cells has 2×10⁹ T regulatory cells, e.g., CD25+ cells, or less (e.g., 1×10⁹, 5×10⁸, 1×10⁸, 5×10⁷, 1×10⁷, or less CD25+ cells).

In one embodiment, the T regulatory cells, e.g., CD25+ cells, are removed from the population using the CliniMAC system with a depletion tubing set, such as, e.g., tubing 162-01. In one embodiment, the CliniMAC system is run on a depletion setting such as, e.g., DEPLETION2.1.

Without wishing to be bound by a particular theory, decreasing the level of negative regulators of immune cells (e.g., decreasing the number of unwanted immune cells, e.g., T_{REG} cells), in a subject prior to apheresis or during manufacturing of a CAR-expressing cell product can reduce the risk of subject relapse. For example, methods of depleting T_{REG} cells are known in the art. Methods of decreasing T_{REG} cells include, but are not limited to, cyclophosphamide, anti-GITR antibody (an anti-GITR antibody described herein), CD25-depletion, and combinations thereof.

In some embodiments, the manufacturing methods comprise reducing the number of (e.g., depleting) T_{REG} cells prior to manufacturing of the CAR-expressing cell. For example, manufacturing methods comprise contacting the sample, e.g., the apheresis sample, with an anti-GITR antibody and/or an anti-CD25 antibody (or fragment thereof, or a CD25-binding ligand), e.g., to deplete T_{REG} cells prior to manufacturing of the CAR-expressing cell (e.g., T cell, NK cell) product.

In an embodiment, a subject is pre-treated with one or more therapies that reduce T_{REG} cells prior to collection of cells for CAR-expressing cell product manufacturing, thereby reducing the risk of subject relapse to CAR-expressing cell treatment. In an embodiment, methods of decreasing T_{REG} cells include, but are not limited to, administration to the subject of one or more of cyclophosphamide, anti-GITR antibody, CD25-depletion, or a combination thereof. Administration of one or more of cyclophosphamide, anti-GITR antibody, CD25-depletion, or a combination thereof, can occur before, during or after an infusion of the CAR-expressing cell product.

In an embodiment, a subject is pre-treated with cyclophosphamide prior to collection of cells for CAR-expressing cell product manufacturing, thereby reducing the risk of subject relapse to CAR-expressing cell treatment. In an embodiment, a subject is pre-treated with an anti-GITR antibody prior to collection of cells for CAR-expressing cell product manufacturing, thereby reducing the risk of subject relapse to CAR-expressing cell treatment.

In one embodiment, the population of cells to be removed are neither the regulatory T cells or tumor cells, but cells that otherwise negatively affect the expansion and/or function of CART cells, e.g. cells expressing CD14, CD11b, CD33, CD15, or other markers expressed by potentially immune suppressive cells. In one embodiment, such cells are envisioned to be removed concurrently with regulatory T cells and/or tumor cells, or following said depletion, or in another order.

The methods described herein can include more than one selection step, e.g., more than one depletion step. Enrichment of a T cell population by negative selection can be accomplished, e.g., with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail can include antibodies to CD14, CD20, CD11b, CD16, HLA-DR, and CD8.

The methods described herein can further include removing cells from the population which express a tumor antigen, e.g., a tumor antigen that does not comprise CD25, e.g., CD19, CD30, CD38, CD123, CD20, CD14 or CD11b, to thereby provide a population of T regulatory depleted, e.g., CD25+ depleted, and tumor antigen depleted cells that are suitable for expression of a CAR, e.g., a CAR described herein. In one embodiment, tumor antigen expressing cells are removed simultaneously with the T regulatory, e.g., CD25+ cells. For example, an anti-CD25 antibody, or fragment thereof, and an anti-tumor antigen antibody, or fragment thereof, can be attached to the same substrate, e.g., bead, which can be used to remove the cells or an anti-CD25 antibody, or fragment thereof, or the anti-tumor antigen antibody, or fragment thereof, can be attached to separate beads, a mixture of which can be used to remove the cells. In other embodiments, the removal of T regulatory cells, e.g., CD25+ cells, and the removal of the tumor antigen expressing cells is sequential, and can occur, e.g., in either order.

Also provided are methods that include removing cells from the population which express a check point inhibitor, e.g., a check point inhibitor described herein, e.g., one or more of PD1+ cells, LAG3+ cells, and TIM3+ cells, to thereby provide a population of T regulatory depleted, e.g., CD25+ depleted cells, and check point inhibitor depleted cells, e.g., PD1+, LAG3+ and/or TIM3+ depleted cells. Exemplary check point inhibitors include B7-H1, B7-1, CD160, P1H, 2B4, PD1, TIM3, CEACAM (e.g., CEACAM-1, CEACAM-3 and/or CEACAM-5), LAG3, TIGIT, CTLA-4, BTLA and LAIR1. In one embodiment, check point inhibitor expressing cells are removed simultaneously with the T regulatory, e.g., CD25+ cells. For example, an anti-CD25 antibody, or fragment thereof, and an anti-check point inhibitor antibody, or fragment thereof, can be attached to the same bead which can be used to remove the cells, or an anti-CD25 antibody, or fragment thereof, and the anti-check point inhibitor antibody, or fragment there, can be attached to separate beads, a mixture of which can be used to remove the cells. In other embodiments, the removal of T regulatory cells, e.g., CD25+ cells, and the removal of the check point inhibitor expressing cells is sequential, and can occur, e.g., in either order.

In one embodiment, a T cell population can be selected that expresses one or more of IFN-γ, TNFα, IL-17A, IL-2, IL-3, IL-4, GM-CSF, IL-10, IL-13, granzyme B, and perforin, or other appropriate molecules, e.g., other cytokines. Methods for screening for cell expression can be determined, e.g., by the methods described in PCT Publication No.: WO 2013/126712.

For isolation of a desired population of cells by positive or negative selection, the concentration of cells and surface (e.g., particles such as beads) can be varied. In certain aspects, it may be desirable to significantly decrease the volume in which beads and cells are mixed together (e.g., increase the concentration of cells), to ensure maximum contact of cells and beads. For example, in one aspect, a concentration of 2 billion cells/ml is used. In one aspect, a concentration of 1 billion cells/ml is used. In a further aspect, greater than 100 million cells/ml is used. In a further aspect, a concentration of cells of 10, 15, 20, 25, 30, 35, 40, 45, or 50 million cells/ml is used. In yet one aspect, a concentration of cells from 75, 80, 85, 90, 95, or 100 million cells/ml is used. In further aspects, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (e.g., leukemic blood, tumor tissue, etc.). Such populations of cells may have therapeutic value and would be desirable to obtain. For example, using high concentration of cells allows more efficient selection of CD8+ T cells that normally have weaker CD28 expression.

In a related aspect, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one aspect, the concentration of cells used is 5×10e6/ml. In other aspects, the concentration used can be from about 1×10⁵/ml to 1×10⁶/ml, and any integer value in between.

In other aspects, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In certain aspects, cryopreserved cells are thawed and washed as described herein and allowed to rest for one hour at room temperature prior to activation using the methods of the present disclosure.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one aspect a blood sample or an apheresis is taken from a generally healthy subject. In certain aspects, a blood sample or an apheresis is taken from a generally healthy subject who is at risk of developing a disease, but who has not yet developed a disease, and the cells of interest are isolated and frozen for later use. In certain aspects, the T cells may be expanded, frozen, and used at a later time. In certain aspects, samples are collected from a patient shortly after diagnosis of a particular disease as described herein but prior to any treatments. In a further aspect, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation.

In a further aspect of the present disclosure, T cells are obtained from a patient directly following treatment that leaves the subject with functional T cells. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present disclosure to collect blood cells, including T cells, dendritic cells, or other cells of the hematopoietic lineage, during this recovery phase. Further, in certain aspects, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

In one embodiment, a T cell population is diaglycerol kinase (DGK)-deficient. DGK-deficient cells include cells that do not express DGK RNA or protein, or have reduced or inhibited DGK activity. DGK-deficient cells can be generated by genetic approaches, e.g., administering RNA-interfering agents, e.g., siRNA, shRNA, miRNA, to reduce or prevent DGK expression. Alternatively, DGK-deficient cells can be generated by treatment with DGK inhibitors described herein.

In one embodiment, a T cell population is Ikaros-deficient. Ikaros-deficient cells include cells that do not express Ikaros RNA or protein, or have reduced or inhibited Ikaros activity, Ikaros-deficient cells can be generated by genetic approaches, e.g., administering RNA-interfering agents, e.g., siRNA, shRNA, miRNA, to reduce or prevent Ikaros expression. Alternatively, Ikaros-deficient cells can be generated by treatment with Ikaros inhibitors, e.g., lenalidomide.

In embodiments, a T cell population is DGK-deficient and Ikaros-deficient, e.g., does not express DGK and Ikaros, or has reduced or inhibited DGK and Ikaros activity. Such DGK and Ikaros-deficient cells can be generated by any of the methods described herein.

In an embodiment, the NK cells are obtained from the subject. In another embodiment, the NK cells are an NK cell line, e.g., NK-92 cell line (Conkwest).

### Allogeneic CAR Immune Effector Cells

In embodiments described herein, the immune effector cell can be an allogeneic immune effector cell, e.g., T cell or NK cell. For example, the cell can be an allogeneic T cell, e.g., an allogeneic T cell lacking expression of a functional T cell receptor (TCR) and/or human leukocyte antigen (HLA), e.g., HLA class I and/or HLA class II.

A T cell lacking a functional TCR can be, e.g., engineered such that it does not express any functional TCR on its surface, engineered such that it does not express one or more subunits that comprise a functional TCR or engineered such that it produces very little functional TCR on its surface. Alternatively, the T cell can express a substantially impaired TCR, e.g., by expression of mutated or truncated forms of one or more of the subunits of the TCR. The term “substantially impaired TCR” means that this TCR will not elicit an adverse immune reaction in a host.

A T cell described herein can be, e.g., engineered such that it does not express a functional HLA on its surface. For example, a T cell described herein, can be engineered such that cell surface expression HLA, e.g., HLA class I and/or HLA class II, is downregulated.

In some embodiments, the T cell can lack a functional TCR and a functional HLA, e.g., HLA class I and/or HLA class II.

Modified T cells that lack expression of a functional TCR and/or HLA can be obtained by any suitable means, including a knock out or knock down of one or more subunit of TCR or HLA. For example, the T cell can include a knock down of TCR and/or HLA using siRNA, shRNA, clustered regularly interspaced short palindromic repeats (CRISPR) transcription-activator like effector nuclease (TALEN), or zinc finger endonuclease (ZFN).

In some embodiments, the allogeneic cell can be a cell which does not expresses or expresses at low levels an inhibitory molecule, e.g. by any method described herein. For example, the cell can be a cell that does not express or expresses at low levels an inhibitory molecule, e.g., that can decrease the ability of a CAR-expressing cell to mount an immune effector response. Examples of inhibitory molecules include PD1, PD-L1, CTLA4, TIM3, LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta. Inhibition of an inhibitory molecule, e.g., by inhibition at the DNA, RNA or protein level, can optimize a CAR-expressing cell performance. In embodiments, an inhibitory nucleic acid, e.g., an inhibitory nucleic acid, e.g., a dsRNA, e.g., an siRNA or shRNA, a clustered regularly interspaced short palindromic repeats (CRISPR), a transcription-activator like effector nuclease (TALEN), or a zinc finger endonuclease (ZFN), e.g., as described herein, can be used.

siRNA and shRNA to Inhibit TCR or HLA

In some embodiments, TCR expression and/or HLA expression can be inhibited using siRNA or shRNA that targets a nucleic acid encoding a TCR and/or HLA, and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), in a cell, e.g., T cell.

Expression systems for siRNA and shRNAs, and exemplary shRNAs, are described, e.g., in paragraphs 649 and 650 of International Publication WO2015/142675, filed Mar. 13, 2015, which is incorporated by reference in its entirety.

### CRISPR to Inhibit TCR or HLA

“CRISPR” or “CRISPR to TCR and/or HLA” or “CRISPR to inhibit TCR and/or HLA” as used herein refers to a set of clustered regularly interspaced short palindromic repeats, or a system comprising such a set of repeats. “Cas”, as used herein, refers to a CRISPR-associated protein.

A “CRISPR/Cas” system refers to a system derived from CRISPR and Cas which can be used to silence or mutate a TCR and/or HLA gene, and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), in a cell, e.g., T cell.

The CRISPR/Cas system, and uses thereof, are described, e.g., in paragraphs 651-658 of International Publication WO2015/142675, filed Mar. 13, 2015, which is incorporated by reference in its entirety.

TALEN to Inhibit TCR and/or HLA

“TALEN” or “TALEN to HLA and/or TCR” or “TALEN to inhibit HLA and/or TCR” refers to a transcription activator-like effector nuclease, an artificial nuclease which can be used to edit the HLA and/or TCR gene, and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), in a cell, e.g., T cell.

TALENs, and uses thereof, are described, e.g., in paragraphs 659-665 of International Publication WO2015/142675, filed Mar. 13, 2015, which is incorporated by reference in its entirety.

Zinc Finger Nuclease to Inhibit HLA and/or TCR

“ZFN” or “Zinc Finger Nuclease” or “ZFN to HLA and/or TCR” or “ZFN to inhibit HLA and/or TCR” refer to a zinc finger nuclease, an artificial nuclease which can be used to edit the HLA and/or TCR gene, and/or an inhibitory molecule described herein (e.g., PD1, PD-L1, PD-L2, CTLA4, TIM3, CEACAM (e.g., CEACAM-1, CEACAM-3 and/or CEACAM-5), LAGS, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGF beta), in a cell, e.g., T cell.

ZFNs, and uses thereof, are described, e.g., in paragraphs 666-671 of International Publication WO2015/142675, filed Mar. 13, 2015, which is incorporated by reference in its entirety.

### Telomerase Expression

While not wishing to be bound by any particular theory, in some embodiments, a therapeutic T cell has short term persistence in a patient, due to shortened telomeres in the T cell; accordingly, transfection with a telomerase gene can lengthen the telomeres of the T cell and improve persistence of the T cell in the patient. See Carl June, “Adoptive T cell therapy for cancer in the clinic”, Journal of Clinical Investigation, 117:1466-1476 (2007). Thus, in an embodiment, an immune effector cell, e.g., a T cell, ectopically expresses a telomerase subunit, e.g., the catalytic subunit of telomerase, e.g., TERT, e.g., hTERT. In some aspects, this disclosure provides a method of producing a CAR-expressing cell, comprising contacting a cell with a nucleic acid encoding a telomerase subunit, e.g., the catalytic subunit of telomerase, e.g., TERT, e.g., hTERT. The cell may be contacted with the nucleic acid before, simultaneous with, or after being contacted with a construct encoding a CAR.

In one aspect, the disclosure features a method of making a population of immune effector cells (e.g., T cells, NK cells). In an embodiment, the method comprises: providing a population of immune effector cells (e.g., T cells or NK cells), contacting the population of immune effector cells with a nucleic acid encoding a CAR; and contacting the population of immune effector cells with a nucleic acid encoding a telomerase subunit, e.g., hTERT, under conditions that allow for CAR and telomerase expression.

In an embodiment, the nucleic acid encoding the telomerase subunit is DNA. In an embodiment, the nucleic acid encoding the telomerase subunit comprises a promoter capable of driving expression of the telomerase subunit.

In an embodiment, hTERT has the amino acid sequence of GenBank Protein ID AAC51724.1 (Meyerson et al., “hEST2, the Putative Human Telomerase Catalytic Subunit Gene, Is Up-Regulated in Tumor Cells and during Immortalization” Cell Volume 90, Issue 4, 22 Aug. 1997, Pages 785-795) as provided in SEQ ID NO: 135.

In an embodiment, the hTERT has a sequence at least 80%, 85%, 90%, 95%, 96\hat{ }, 97%, 98%, or 99% identical to the sequence of SEQ ID NO: 135. In an embodiment, the hTERT has a sequence of SEQ ID NO: 135. In an embodiment, the hTERT comprises a deletion (e.g., of no more than 5, 10, 15, 20, or 30 amino acids) at the N-terminus, the C-terminus, or both. In an embodiment, the hTERT comprises a transgenic amino acid sequence (e.g., of no more than 5, 10, 15, 20, or 30 amino acids) at the N-terminus, the C-terminus, or both.

In an embodiment, the hTERT is encoded by the nucleic acid sequence of GenBank Accession No. AF018167 (Meyerson et al., “hEST2, the Putative Human Telomerase Catalytic Subunit Gene, Is Up-Regulated in Tumor Cells and during Immortalization” Cell Volume 90, Issue 4, 22 Aug. 1997, Pages 785-795) as provided in SEQ ID NO: 136

In an embodiment, the hTERT is encoded by a nucleic acid having a sequence at least 80%, 85%, 90%, 95%, 96, 97%, 98%, or 99% identical to the sequence of SEQ ID NO: 136. In an embodiment, the hTERT is encoded by a nucleic acid of SEQ ID NO: 136.

Activation and Expansion of Immune Effector Cells (e.g., T Cells)

Immune effector cells, such as T cells, may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, the T cells of the invention may be expanded by contact with a surface having attached thereto an agent that stimulates a CD3/TCR complex associated signal and a ligand that stimulates a costimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In certain aspects, the primary stimulatory signal and the costimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one aspect, the agent providing the costimulatory signal is bound to a cell surface and the agent providing the primary activation signal is in solution or coupled to a surface. In certain aspects, both agents can be in solution. In one aspect, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In one aspect, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the costimulatory signal is an anti-CD28 antibody or antigen-binding fragment thereof; and both agents are co-immobilized to the same bead in equivalent molecular amounts. In one aspect, a 1:1 ratio of each antibody bound to the beads for CD4+ T cell expansion and T cell growth is used. In certain aspects of the present invention, a ratio of anti CD3:CD28 antibodies bound to the beads is used such that an increase in T cell expansion is observed as compared to the expansion observed using a ratio of 1:1. In one particular aspect an increase of from about 1 to about 3 fold is observed as compared to the expansion observed using a ratio of 1:1. In one aspect, the ratio of CD3:CD28 antibody bound to the beads ranges from 100:1 to 1:100 and all integer values there between. In one aspect of the present invention, more anti-CD28 antibody is bound to the particles than anti-CD3 antibody, i.e., the ratio of CD3:CD28 is less than one. In certain aspects of the invention, the ratio of anti CD28 antibody to anti CD3 antibody bound to the beads is greater than 2:1. In one particular aspect, a 1:100 CD3:CD28 ratio of antibody bound to beads is used. In one aspect, a 1:75 CD3:CD28 ratio of antibody bound to beads is used. In a further aspect, a 1:50 CD3:CD28 ratio of antibody bound to beads is used. In one aspect, a 1:30 CD3:CD28 ratio of antibody bound to beads is used. In one preferred aspect, a 1:10 CD3:CD28 ratio of antibody bound to beads is used. In one aspect, a 1:3 CD3:CD28 ratio of antibody bound to the beads is used. In yet one aspect, a 3:1 CD3:CD28 ratio of antibody bound to the beads is used.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain aspects the ratio of cells to particles ranges from 1:100 to 100:1 and any integer values in-between and in further aspects the ratio comprises 1:9 to 9:1 and any integer values in between, can also be used to stimulate T cells. The ratio of anti-CD3- and anti-CD28-coupled particles to T cells that result in T cell stimulation can vary as noted above, however certain preferred values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one preferred ratio being at least 1:1 particles per T cell. In one aspect, a ratio of particles to cells of 1:1 or less is used. In one particular aspect, a preferred particle: cell ratio is 1:5. In further aspects, the ratio of particles to cells can be varied depending on the day of stimulation. For example, in one aspect, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In one particular aspect, the ratio of particles to cells is 1:1 on the first day of stimulation and adjusted to 1:5 on the third and fifth days of stimulation. In one aspect, particles are added on a daily or every other day basis to a final ratio of 1:1 on the first day, and 1:5 on the third and fifth days of stimulation. In one aspect, the ratio of particles to cells is 2:1 on the first day of stimulation and adjusted to 1:10 on the third and fifth days of stimulation. In one aspect, particles are added on a daily or every other day basis to a final ratio of 1:1 on the first day, and 1:10 on the third and fifth days of stimulation. One of skill in the art will appreciate that a variety of other ratios may be suitable for use in the present invention. In particular, ratios will vary depending on particle size and on cell size and type. In one aspect, the most typical ratios for use are in the neighborhood of 1:1, 2:1 and 3:1 on the first day.

In further aspects of the present invention, the cells, such as T cells, are combined with agent-coated beads, the beads and the cells are subsequently separated, and then the cells are cultured. In an alternative aspect, prior to culture, the agent-coated beads and cells are not separated but are cultured together. In a further aspect, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In one aspect the cells (for example, 10\hat{ }4 to 10\hat{ }9 T cells) and beads (for example, DYNABEADS® M-450 CD3/CD28 T paramagnetic beads at a ratio of 1:1) are combined in a buffer, for example PBS (without divalent cations such as, calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present invention. In certain aspects, it may be desirable to significantly decrease the volume in which particles and cells are mixed together (i.e., increase the concentration of cells), to ensure maximum contact of cells and particles. For example, in one aspect, a concentration of about 2 billion cells/ml is used. In one aspect, greater than 100 million cells/ml is used. In a further aspect, a concentration of cells of 10, 15, 20, 25, 30, 35, 40, 45, or 50 million cells/ml is used. In yet one aspect, a concentration of cells from 75, 80, 85, 90, 95, or 100 million cells/ml is used. In further aspects, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in certain aspects. For example, using high concentration of cells allows more efficient selection of CD8+ T cells that normally have weaker CD28 expression.

In one aspect of the present invention, the mixture may be cultured for several hours (about 3 hours) to about 14 days or any hourly integer value in between. In one aspect, the mixture may be cultured for 21 days. In one aspect of the invention the beads and the T cells are cultured together for about eight days. In one aspect, the beads and T cells are cultured together for 2-3 days. Several cycles of stimulation may also be desired such that culture time of T cells can be 60 days or more. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, a-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

T cells that have been exposed to varied stimulation times may exhibit different characteristics. For example, typical blood or apheresed peripheral blood mononuclear cell products have a helper T cell population (TH, CD4+) that is greater than the cytotoxic or suppressor T cell population (TC, CD8+). Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

Once an EGFRvIII CAR is constructed, various assays can be used to evaluate the activity of the molecule, such as but not limited to, the ability to expand T cells following antigen stimulation, sustain T cell expansion in the absence of re-stimulation, and anti-cancer activities in appropriate in vitro and animal models. Assays to evaluate the effects of an EGFRvIII CAR are described in further detail below

Western blot analysis of CAR expression in primary T cells can be used to detect the presence of monomers and dimers. See, e.g., Milone et al., Molecular Therapy 17(8): 1453-1464 (2009). Very briefly, T cells (1:1 mixture of CD4⁺ and CD8⁺ T cells) expressing the CARs are expanded in vitro for more than 10 days followed by lysis and SDS-PAGE under reducing conditions. CARs containing the full length TCR-t cytoplasmic domain and the endogenous TCR-ζ chain are detected by western blotting using an antibody to the TCR-ζ chain. The same T cell subsets are used for SDS-PAGE analysis under non-reducing conditions to permit evaluation of covalent dimer formation.

In vitro expansion of CARP T cells following antigen stimulation can be measured by flow cytometry. For example, a mixture of CD4⁺ and CD8⁺ T cells are stimulated with αCD3/αCD28 aAPCs followed by transduction with lentiviral vectors expressing GFP under the control of the promoters to be analyzed. Exemplary promoters include the CMV IE gene, EF-1α, ubiquitin C, or phosphoglycerokinase (PGK) promoters. GFP fluorescence is evaluated on day 6 of culture in the CD4+ and/or CD8+ T cell subsets by flow cytometry. See, e.g., Milone et al., Molecular Therapy 17(8): 1453-1464 (2009). Alternatively, a mixture of CD4⁺ and CD8⁺ T cells are stimulated with αCD3/αCD28 coated magnetic beads on day 0, and transduced with CAR on day 1 using a bicistronic lentiviral vector expressing CAR along with eGFP using a 2A ribosomal skipping sequence. Cultures are re-stimulated with either EGFRvIII⁺ U-87 cells (U-87-EGFRvIII), wild-type U-87 cells (U-87 wild type) or K562 cells expressing hCD32 and 4-1BBL in the presence of antiCD3 and anti-CD28 antibody (K562-BBL-3/28) following washing. Exogenous IL-2 is added to the cultures every other day at 100 IU/ml. GFP T cells are enumerated by flow cytometry using bead-based counting. See, e.g., Milone et al., Molecular Therapy 17(8): 1453-1464 (2009).

Sustained CAR⁺ T cell expansion in the absence of re-stimulation can also be measured. See, e.g., Milone et al., Molecular Therapy 17(8): 1453-1464 (2009). Briefly, mean T cell volume (fl) is measured on day 8 of culture using a Coulter Multisizer III particle counter following stimulation with αCD3/αCD28 coated magnetic beads on day 0, and transduction with the indicated CAR on day 1.

Assessment of cell proliferation and cytokine production has been previously described, e.g., at Milone et al., Molecular Therapy 17(8): 1453-1464 (2009). Briefly, assessment of CAR-mediated proliferation is performed in microtiter plates by mixing washed T cells with target cells, such asU87MG, BHK or CHO cells expressing EGFRvIII or EGFR wildtype (wt) or CD32 and CD137 (KT32-BBL) for a final T-cell:target cell ratio of 1:1. Anti-CD3 (clone OKT3) and anti-CD28 (clone 9.3) monoclonal antibodies are added to cultures with KT32-BBL cells to serve as a positive control for stimulating T-cell proliferation since these signals support long-term CD8⁺ T cell expansion ex vivo. T cells are enumerated in cultures using CountBright™ fluorescent beads (Invitrogen, Carlsbad, Calif.) and flow cytometry as described by the manufacturer. CARP T cells are identified by GFP expression using T cells that are engineered with eGFP-2A linked CAR-expressing lentiviral vectors. For CAR+ T cells not expressing GFP, the CAR+ T cells are detected with biotinylated recombinant EGFRvIII protein and a secondary avidin-PE conjugate. CD4+ and CD8⁺ expression on T cells are also simultaneously detected with specific monoclonal antibodies (BD Biosciences). Cytokine measurements are performed on supernatants collected 24 hours following re-stimulation using the human TH1/TH2 cytokine cytometric bead array kit (BD Biosciences, San Diego, Calif.) according the manufacturer's instructions. Fluorescence is assessed using a FACScalibur flow cytometer, and data is analyzed according to the manufacturer's instructions.

Cytotoxicity can be assessed by a standard 51Cr-release assay. See, e.g., Milone et al., Molecular Therapy 17(8): 1453-1464 (2009). Briefly, target cells (U87MG, BHK or CHO cells expressing EGFRvIII or EGFR wildtype (wt) are loaded with 51Cr (as NaCrO4, New England Nuclear, Boston, Mass.) at 37° C. for 2 hours with frequent agitation, washed twice in complete RPMI and plated into microtiter plates. Effector T cells are mixed with target cells in the wells in complete RPMI at varying ratios of effector cell:target cell (E:T). Additional wells containing media only (spontaneous release, SR) or a 1% solution of triton-X 100 detergent (total release, TR) are also prepared. After 4 hours of incubation at 37° C., supernatant from each well is harvested. Released ⁵¹Cr is then measured using a gamma particle counter (Packard Instrument Co., Waltham, Mass.). Each condition is performed in at least triplicate, and the percentage of lysis is calculated using the formula: % Lysis=(ER−SR)/(TR−SR), where ER represents the average 51Cr released for each experimental condition. Alternative cytotoxicity assays may also be used, such as flow based cytotoxicity assays, as described in Example 8.

Click beetle red and click beetle green luciferase can be used to simultaneously follow tumor progression and T cell trafficking, as each use the same luciferin substrate but emit light at the opposite ends of the visible light spectrum.

Other assays, including those described in the Example section herein as well as those that are known in the art can also be used to evaluate the EGFRvIII CAR constructs of the invention.

### Populations of CAR Cells

In another aspect, the present invention provides a population of CAR-expressing cells, e.g., a population of EGFRvIII CAR-expressing cells. In some embodiments, the population of CAR-expressing cells comprises a mixture of cells expressing different CARs.

For example, in one embodiment, the population of CAR-expressing cells can include a first cell expressing a CAR having an anti-EGFRvIII binding domain described herein, and a second cell expressing a CAR having a different anti-EGFRvIII binding domain, e.g., an anti-EGFRvIII binding domain described herein that differs from the anti-EGFRvIII binding domain in the CAR expressed by the first cell.

As another example, the population of CAR-expressing cells can include a first cell expressing a CAR that includes an anti-EGFRvIII binding domain, e.g., as described herein, and a second cell expressing a CAR that includes an antigen binding domain to a target other than EGFRvIII. In one embodiment, the population of CAR-expressing cells includes, e.g., a first cell expressing a CAR that includes a primary intracellular signaling domain, and a second cell expressing a CAR that includes a secondary signaling domain.

In another aspect, the present invention provides a population of cells wherein at least one cell in the population expresses a CAR having an anti-EGFRvIII binding domain described herein, and a second cell expressing another agent, e.g., an agent which enhances the activity or function of a CAR-expressing cell. For example, in one embodiment, the agent can be an agent which modulates or regulates, e.g., inhibits, T cell function. In some embodiments, the molecule that modulates or regulates T cell function is an inhibitory molecule, e.g., an agent described herein. Inhibitory molecules, e.g., can, in some embodiments, decrease the ability of a CAR-expressing cell to mount an immune effector response. Examples of inhibitory molecules include PD1, PD-L1, CTLA4, TIM3, LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, or TGF beta. In one embodiment, the agent which inhibits an inhibitory molecule comprises a first polypeptide, e.g., an inhibitory molecule, associated with a second polypeptide that provides a positive signal to the cell, e.g., an intracellular signaling domain described herein. In one embodiment, the agent comprises a first polypeptide, e.g., of an inhibitory molecule such as PD1, PD-L1, CTLA4, TIM3, LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, or TGF beta, or a fragment of any of these (e.g., at least a portion of an extracellular domain of any of these), and a second polypeptide which is an intracellular signaling domain described herein (e.g., comprising a costimulatory domain (e.g., 4-1BB, CD27, CD28, or ICOS, e.g., as described herein) and/or a primary signaling domain (e.g., a CD3 zeta signaling domain described herein). In one embodiment, the agent comprises a first polypeptide of PD1 or a fragment thereof (e.g., at least a portion of the extracellular domain of PD1), and a second polypeptide of an intracellular signaling domain described herein (e.g., a CD28 signaling domain described herein and/or a CD3 zeta signaling domain described herein).

In one aspect, the present invention provides methods comprising administering a population of CAR-expressing cells, e.g., CART cells, e.g., a mixture of cells expressing different CARs, in combination with another agent, e.g., a PD-1 inhibitor, such as a PD-1 inhibitor described herein. In another aspect, the present invention provides methods comprising administering a population of cells wherein at least one cell in the population expresses a CAR having an anti-EGFRvIII binding domain as described herein, and a second cell expressing another agent, e.g., an agent which enhances the activity or fitness of a CAR-expressing cell, in combination with another agent, e.g., a PD-1 inhibitor, such as a PD-1 inhibitor described herein.

### PD-1 Inhibitors

The immune system has the capability of recognizing and eliminating tumor cells; however, tumors can use multiple strategies to evade immunity. Blockade of immune checkpoints is an approach to activating or reactivating therapeutic antitumor immunity. PD-1 is an exemplary immune checkpoint molecule.

PD-1 is a CD28/CTLA-4 family member expressed, e.g., on activated CD4⁺ and CD8⁺ T cells, T_{regs}, and B cells. See, e.g., Agata et al. 1996 Int. Immunol 8:765-75. PD-1 is an inhibitory member of the CD28 family of receptors that also includes CD28, CTLA-4, ICOS, and BTLA. PD-1 negatively regulates effector T cell signaling and function. PD-1 is induced on tumor-infiltrating T cells, and can result in functional exhaustion or dysfunction (Keir et al. (2008) Annu. Rev. Immunol. 26:677-704; Pardoll et al. (2012) Nat Rev Cancer 12(4):252-64). PD-1 delivers a coinhibitory signal upon binding to either of its two ligands, Programmed Death-Ligand 1 (PD-L1) or Programmed Death-Ligand 2 (PD-L2). PD-L1 and PD-L2 have been shown to downregulate T cell activation upon binding to PD-1 (Freeman et a. 2000 J Exp Med 192:1027-34; Latchman et al. 2001 Nat Immunol 2:261-8; Carter et al. 2002 Eur J Immunol 32:634-43). PD-L1 is expressed on a number of cell types, including T cells, natural killer (NK) cells, macrophages, dendritic cells (DCs), B cells, epithelial cells, vascular endothelial cells, as well as many types of tumors. PD-L1 is abundant in human cancers (Dong et al. 2003 J Mol Med 81:281-7; Blank et al. 2005 Cancer Immunol. Immunother 54:307-314; Konishi et al. 2004 Clin Cancer Res 10:5094), and high expression of PD-L1 on murine and human tumors has been linked to poor clinical outcomes in a variety of cancers (Keir et al. (2008) Annu. Rev. Immunol. 26:677-704; Pardoll et al. (2012) Nat Rev Cancer 12(4):252-64). PD-L2 is expressed on dendritic cells, macrophages, and some tumors. Blockade of the PD-1 pathway has been pre-clinically and clinically validated for cancer immunotherapy. Immune suppression can be reversed by inhibiting the local interaction of PD-1 with PD-L1. Both preclinical and clinical studies have demonstrated that anti-PD-1 blockade can restore activity of effector T cells and results in robust anti-tumor response. For example, blockade of PD-1 pathway can restore exhausted/dysfunctional effector T cell function (e.g., proliferation, IFN-γ secretion, or cytolytic function) and/or inhibit T_{reg} cell function (Keir et al. (2008) Annu. Rev. Immunol. 26:677-704; Pardoll et al. (2012) Nat Rev Cancer 12(4):252-64). Blockade of the PD-1 pathway can be affected with an antibody, an antigen binding fragment thereof, an immunoadhesin, a fusion protein, or oligopeptide of PD-1, PD-L1 and/or PD-L2.

Antibody Molecules to PD-1

In one embodiment, the PD-1 inhibitor is an anti-PD-1 antibody molecule as described in US 2015/0210769, published on Jul. 30, 2015, entitled “Antibody Molecules to PD-1 and Uses Thereof,” incorporated by reference in its entirety.

In some embodiments, the anti-PD-1 antibody molecule (e.g., an isolated or recombinant antibody molecule) has one or more of the following properties:

(i) binds to PD-1, e.g., human PD-1, with high affinity, e.g., with an affinity constant of at least about 10⁷ M⁻¹, typically about 10⁸ M⁻¹, and more typically, about 10⁹ M⁻¹ to 10¹⁰ M⁻¹ or stronger;

(ii) does not substantially bind to CD28, CTLA-4, ICOS or BTLA;

(iii) inhibits or reduces binding of PD-1 to a PD-1 ligand, e.g., PD-L1 or PD-L2, or both;

(iv) binds specifically to an epitope on PD-1, e.g., the same or similar epitope as the epitope recognized by murine monoclonal antibody BAP049 or a chimeric antibody BAP049, e.g., BAP049-chi or BAP049-chi-Y;

(v) shows the same or similar binding affinity or specificity, or both, as any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E;

(vi) shows the same or similar binding affinity or specificity, or both, as an antibody molecule (e.g., an heavy chain variable region and light chain variable region) described in Table 6;

(vii) shows the same or similar binding affinity or specificity, or both, as an antibody molecule (e.g., an heavy chain variable region and light chain variable region) having an amino acid sequence shown in Table 6;

(viii) shows the same or similar binding affinity or specificity, or both, as an antibody molecule (e.g., an heavy chain variable region and light chain variable region) encoded by the nucleotide sequence shown in Table 6;

(ix) inhibits, e.g., competitively inhibits, the binding of a second antibody molecule to PD-1, wherein the second antibody molecule is an antibody molecule described herein, e.g., an antibody molecule chosen from, e.g., any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E;

(x) binds the same or an overlapping epitope with a second antibody molecule to PD-1, wherein the second antibody molecule is an antibody molecule described herein, e.g., an antibody molecule chosen from, e.g., any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E;

(xi) competes for binding, and/or binds the same epitope, with a second antibody molecule to PD-1, wherein the second antibody molecule is an antibody molecule described herein, e.g., an antibody molecule chosen from, e.g., any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E;

(xii) has one or more biological properties of an antibody molecule described herein, e.g., an antibody molecule chosen from, e.g., any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E;

(xiii) has one or more pharmacokinetic properties of an antibody molecule described herein, e.g., an antibody molecule chosen from, e.g., any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E;

(xiv) inhibits one or more activities of PD-1, e.g., results in one or more of: an increase in tumor infiltrating lymphocytes, an increase in T-cell receptor mediated proliferation, or a decrease in immune evasion by cancerous cells;

(xv) binds human PD-1 and is cross-reactive with cynomolgus PD-1;

(xvi) binds to one or more residues within the C strand, CC′ loop, C′ strand, or FG loop of PD-1, or a combination two, three or all of the C strand, CC′ loop, C′ strand or FG loop of PD-1, e.g., wherein the binding is assayed using ELISA or Biacore; or

(xvii) has a VL region that contributes more to binding to PD-1 than a VH region.

In some embodiments, the antibody molecule binds to PD-1 with high affinity, e.g., with a K_{D} that is about the same, or at least about 10%, 20%, 30%, 40%, 50%, 60%, 70%, 80% or 90% higher or lower than the K_{D} of a murine or chimeric anti-PD-1 antibody molecule, e.g., a murine or chimeric anti-PD-1 antibody molecule described herein. In some embodiments, the K_{D} of the murine or chimeric anti-PD-1 antibody molecule is less than about 0.4, 0.3, 0.2, 0.1, or 0.05 nM, e.g., measured by a Biacore method. In some embodiments, the K_{D} of the murine or chimeric anti-PD-1 antibody molecule is less than about 0.2 nM, e.g., about 0.135 nM. In other embodiments, the K_{D} of the murine or chimeric anti PD-1 antibody molecule is less than about 10, 5, 3, 2, or 1 nM, e.g., measured by binding on cells expressing PD-1 (e.g., 300.19 cells). In some embodiments, the K_{D} of the murine or chimeric anti PD-1 antibody molecule is less than about 5 nM, e.g., about 4.60 nM (or about 0.69 μg/mL).

In some embodiments, the anti-PD-1 antibody molecule binds to PD-1 with a K_{off} slower than 1×10⁴, 5×10⁻⁵, or 1×10⁻⁵ s⁻¹, e.g., about 1.65×10⁻⁵ s⁻¹. In some embodiments, the anti-PD-1 antibody molecule binds to PD-1 with a Kₒₙ faster than 1×10⁴, 5×10⁴, 1×10⁵, or 5×10⁵ M⁻¹ s⁻¹, e.g., about 1.23×10⁵ M⁻¹ s⁻¹.

In some embodiments, the expression level of the antibody molecule is higher, e.g., at least about 0.5, 1, 2, 3, 4, 5, 6, 7, 8, 9 or 10-fold higher, than the expression level of a murine or chimeric antibody molecule, e.g., a murine or chimeric anti-PD-1 antibody molecule described herein. In some embodiments, the antibody molecule is expressed in CHO cells.

In some embodiments, the anti-PD-1 antibody molecule reduces one or more PD-1-associated activities with an IC₅₀ (concentration at 50% inhibition) that is about the same or lower, e.g., at least about 10%, 20%, 30%, 40%, 50%, 60%, 70%, 80% or 90% lower, than the IC₅₀ of a murine or chimeric anti-PD-1 antibody molecule, e.g., a murine or chimeric anti-PD-1 antibody molecule described herein. In some embodiments, the IC₅₀ of the murine or chimeric anti-PD-1 antibody molecule is less than about 6, 5, 4, 3, 2, or 1 nM, e.g., measured by binding on cells expressing PD-1 (e.g., 300.19 cells). In some embodiments, the IC₅₀ of the murine or chimeric anti-PD-1 antibody molecule is less than about 4 nM, e.g., about 3.40 nM (or about 0.51 μg/mL). In some embodiments, the PD-1-associated activity reduced is the binding of PD-L1 and/or PD-L2 to PD-1. In some embodiments, the anti-PD-1 antibody molecule binds to peripheral blood mononucleated cells (PBMCs) activated by Staphylococcal enterotoxin B (SEB). In other embodiments, the anti-PD-1 antibody molecule increases the expression of IL-2 on whole blood activated by SEB. For example, the anti-PD-1 antibody increases the expression of IL-2 by at least about 2, 3, 4, or 5-fold, compared to the expression of IL-2 when an isotype control (e.g., IgG4) is used.

In some embodiments, the anti-PD-1 antibody molecule has improved stability, e.g., at least about 0.5, 1, 2, 3, 4, 5, 6, 7, 8, 9 or 10-fold more stable in vivo or in vitro, than a murine or chimeric anti-PD-1 antibody molecule, e.g., a murine or chimeric anti-PD-1 antibody molecule described herein.

In one embodiment, the anti-PD-1 antibody molecule is a humanized antibody molecule and has a risk score based on T cell epitope analysis of 300 to 700, 400 to 650, 450 to 600, or a risk score as described herein.

In another embodiment, the anti-PD-1 antibody molecule comprises at least one antigen-binding region, e.g., a variable region or an antigen-binding fragment thereof, from an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 6, or encoded by the nucleotide sequence in Table 6; or a sequence substantially identical (e.g., at least 80%, 85%, 90%, 92%, 95%, 97%, 98%, 99% or higher identical) to any of the aforesaid sequences.

In yet another embodiment, the anti-PD-1 antibody molecule comprises at least one, two, three or four variable regions from an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 6, or encoded by the nucleotide sequence in Table 6; or a sequence substantially identical (e.g., at least 80%, 85%, 90%, 92%, 95%, 97%, 98%, 99% or higher identical) to any of the aforesaid sequences.

In yet another embodiment, the anti-PD-1 antibody molecule comprises at least one or two heavy chain variable regions from an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 6, or encoded by the nucleotide sequence in Table 6; or a sequence substantially identical (e.g., at least 80%, 85%, 90%, 92%, 95%, 97%, 98%, 99% or higher identical) to any of the aforesaid sequences.

In yet another embodiment, the anti-PD-1 antibody molecule comprises at least one or two light chain variable regions from an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 6, or encoded by the nucleotide sequence in Table 6; or a sequence substantially identical (e.g., at least 80%, 85%, 90%, 92%, 95%, 97%, 98%, 99% or higher identical) to any of the aforesaid sequences.

In yet another embodiment, the anti-PD-1 antibody molecule includes a heavy chain constant region for an IgG4, e.g., a human IgG4. In one embodiment, the human IgG4 includes a substitution at position 228 according to EU numbering (e.g., a Ser to Pro substitution). In still another embodiment, the anti-PD-1 antibody molecule includes a heavy chain constant region for an IgG1, e.g., a human IgG1. In one embodiment, the human IgG1 includes a substitution at position 297 according to EU numbering (e.g., an Asn to Ala substitution). In one embodiment, the human IgG1 includes a substitution at position 265 according to EU numbering, a substitution at position 329 according to EU numbering, or both (e.g., an Asp to Ala substitution at position 265 and/or a Pro to Ala substitution at position 329). In one embodiment, the human IgG1 includes a substitution at position 234 according to EU numbering, a substitution at position 235 according to EU numbering, or both (e.g., a Leu to Ala substitution at position 234 and/or a Leu to Ala substitution at position 235). In one embodiment, the heavy chain constant region comprises an amino sequence set forth in Table 2, or a sequence substantially identical (e.g., at least 80%, 85%, 90%, 92%, 95%, 97%, 98%, 99% or higher identical) thereto.

In yet another embodiment, the anti-PD-1 antibody molecule includes a kappa light chain constant region, e.g., a human kappa light chain constant region. In one embodiment, the light chain constant region comprises an amino sequence set forth in Table 3 of US 2015/0210769A1, or a sequence substantially identical (e.g., at least 80%, 85%, 90%, 92%, 95%, 97%, 98%, 99% or higher identical) thereto.

In another embodiment, the anti-PD-1 antibody molecule includes a heavy chain constant region for an IgG4, e.g., a human IgG4, and a kappa light chain constant region, e.g., a human kappa light chain constant region, e.g., a heavy and light chain constant region comprising an amino sequence set forth in Table 3 of US 2015/0210769A1, or a sequence substantially identical (e.g., at least 80%, 85%, 90%, 92%, 95%, 97%, 98%, 99% or higher identical) thereto. In one embodiment, the human IgG4 includes a substitution at position 228 according to EU numbering (e.g., a Ser to Pro substitution). In yet another embodiment, the anti-PD-1 antibody molecule includes a heavy chain constant region for an IgG1, e.g., a human IgG1, and a kappa light chain constant region, e.g., a human kappa light chain constant region, e.g., a heavy and light chain constant region comprising an amino sequence set forth in Table 3 of US 2015/0210769A1, or a sequence substantially identical (e.g., at least 80%, 85%, 90%, 92%, 95%, 97%, 98%, 99% or higher identical) thereto. In one embodiment, the human IgG1 includes a substitution at position 297 according to EU numbering (e.g., an Asn to Ala substitution). In one embodiment, the human IgG1 includes a substitution at position 265 according to EU numbering, a substitution at position 329 according to EU numbering, or both (e.g., an Asp to Ala substitution at position 265 and/or a Pro to Ala substitution at position 329). In one embodiment, the human IgG1 includes a substitution at position 234 according to EU numbering, a substitution at position 235 according to EU numbering, or both (e.g., a Leu to Ala substitution at position 234 and/or a Leu to Ala substitution at position 235).

In another embodiment, the anti-PD-1 antibody molecule includes a heavy chain variable domain and a constant region, a light chain variable domain and a constant region, or both, comprising the amino acid sequence of BAP049-Clone-A, BAP049-Clone-B, BAP049-Clone-C, BAP049-Clone-D, or BAP049-Clone-E; or as described in Table 6, or encoded by the nucleotide sequence in Table 6; or a sequence substantially identical (e.g., at least 80%, 85%, 90%, 92%, 95%, 97%, 98%, 99% or higher identical) to any of the aforesaid sequences. The anti-PD-1 antibody molecule, optionally, comprises a leader sequence from a heavy chain, a light chain, or both, as show in in Table 4 of US 2015/0210769A1; or a sequence substantially identical thereto.

In yet another embodiment, the anti-PD-1 antibody molecule includes at least one, two, or three complementarity determining regions (CDRs) from a heavy chain variable region of an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 6, or encoded by the nucleotide sequence in Table 6; or a sequence substantially identical (e.g., at least 80%, 85%, 90%, 92%, 95%, 97%, 98%, 99% or higher identical) to any of the aforesaid sequences.

In yet another embodiment, the anti-PD-1 antibody molecule includes at least one, two, or three CDRs (or collectively all of the CDRs) from a heavy chain variable region comprising an amino acid sequence shown in Table 6, or encoded by a nucleotide sequence shown in Table 6. In one embodiment, one or more of the CDRs (or collectively all of the CDRs) have one, two, three, four, five, six or more changes, e.g., amino acid substitutions or deletions, relative to the amino acid sequence shown in Table 6, or encoded by a nucleotide sequence shown in Table 6.

In yet another embodiment, the anti-PD-1 antibody molecule includes at least one, two, or three CDRs from a light chain variable region of an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 6, or encoded by the nucleotide sequence in Table 6; or a sequence substantially identical (e.g., at least 80%, 85%, 90%, 92%, 95%, 97%, 98%, 99% or higher identical) to any of the aforesaid sequence.

In yet another embodiment, the anti-PD-1 antibody molecule includes at least one, two, or three CDRs (or collectively all of the CDRs) from a light chain variable region comprising an amino acid sequence shown in Table 6, or encoded by a nucleotide sequence shown in Table 6. In one embodiment, one or more of the CDRs (or collectively all of the CDRs) have one, two, three, four, five, six or more changes, e.g., amino acid substitutions or deletions, relative to the amino acid sequence shown in Table 6, or encoded by a nucleotide sequence shown in Table 6. In certain embodiments, the anti-PD-1 antibody molecule includes a substitution in a light chain CDR, e.g., one or more substitutions in a CDR1, CDR2 and/or CDR3 of the light chain. In one embodiment, the anti-PD-1 antibody molecule includes a substitution in the light chain CDR3 at position 102 of the light variable region, e.g., a substitution of a cysteine to tyrosine, or a cysteine to serine residue, at position 102 of the light variable region according to Table 6 (e.g., SEQ ID NO: 152 or 162 for murine or chimeric, unmodified; or any of SEQ ID NOs: 168, 176, 180, 188, 192, 196, 200, 204, 208, or 212 for a modified sequence).

In another embodiment, the anti-PD-1 antibody molecule includes at least one, two, three, four, five or six CDRs (or collectively all of the CDRs) from a heavy and light chain variable region comprising an amino acid sequence shown in Table 6, or encoded by a nucleotide sequence shown in Table 6. In one embodiment, one or more of the CDRs (or collectively all of the CDRs) have one, two, three, four, five, six or more changes, e.g., amino acid substitutions or deletions, relative to the amino acid sequence shown in Table 6, or encoded by a nucleotide sequence shown in Table 6.

In one embodiment, the anti-PD-1 antibody molecule includes all six CDRs from an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 6, or encoded by the nucleotide sequence in Table 6, or closely related CDRs, e.g., CDRs which are identical or which have at least one amino acid alteration, but not more than two, three or four alterations (e.g., substitutions, deletions, or insertions, e.g., conservative substitutions). In one embodiment, the anti-PD-1 antibody molecule may include any CDR described herein. In certain embodiments, the anti-PD-1 antibody molecule includes a substitution in a light chain CDR, e.g., one or more substitutions in a CDR1, CDR2 and/or CDR3 of the light chain. In one embodiment, the anti-PD-1 antibody molecule includes a substitution in the light chain CDR3 at position 102 of the light variable region, e.g., a substitution of a cysteine to tyrosine, or a cysteine to serine residue, at position 102 of the light variable region according to Table 6 (e.g., SEQ ID NO: 152 or 162 for murine or chimeric, unmodified; or any of SEQ ID NOs: 168, 176, 180, 188, 192, 196, 200, 204, 208, or 212 for a modified sequence).

In another embodiment, the anti-PD-1 antibody molecule includes at least one, two, or three CDRs according to Kabat et al. (e.g., at least one, two, or three CDRs according to the Kabat definition as set out in Table 6) from a heavy chain variable region of an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 6, or encoded by the nucleotide sequence in Table 6; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 6.

In another embodiment, the anti-PD-1 antibody molecule includes at least one, two, or three CDRs according to Kabat et al. (e.g., at least one, two, or three CDRs according to the Kabat definition as set out in Table 6) from a light chain variable region of an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 6, or encoded by the nucleotide sequence in Table 6; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 6.

In yet another embodiment, the anti-PD-1 antibody molecule includes at least one, two, three, four, five, or six CDRs according to Kabat et al. (e.g., at least one, two, three, four, five, or six CDRs according to the Kabat definition as set out in Table 6) from the heavy and light chain variable regions of an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 6, or encoded by the nucleotide sequence in Table 6; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Kabat et al. shown in Table 6.

In yet another embodiment, the anti-PD-1 antibody molecule includes all six CDRs according to Kabat et al. (e.g., all six CDRs according to the Kabat definition as set out in Table 6) from the heavy and light chain variable regions of an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 6, or encoded by the nucleotide sequence in Table 6; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Kabat et al. shown in Table 6. In one embodiment, the anti-PD-1 antibody molecule may include any CDR described herein.

In another embodiment, the anti-PD-1 antibody molecule includes at least one, two, or three Chothia hypervariable loops (e.g., at least one, two, or three hypervariable loops according to the Chothia definition as set out in Table 6) from a heavy chain variable region of an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 6, or encoded by the nucleotide sequence in Table 6; or at least the amino acids from those hypervariable loops that contact PD-1; or which have at least one amino acid alteration, but not more than two, three or four alterations (e.g., substitutions, deletions, or insertions, e.g., conservative substitutions) relative to one, two, or three hypervariable loops according to Chothia et al. shown in Table 6.

In another embodiment, the anti-PD-1 antibody molecule includes at least one, two, or three Chothia hypervariable loops (e.g., at least one, two, or three hypervariable loops according to the Chothia definition as set out in Table 6) of a light chain variable region of an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 6, or encoded by the nucleotide sequence in Table 6; or at least the amino acids from those hypervariable loops that contact PD-1; or which have at least one amino acid alteration, but not more than two, three or four alterations (e.g., substitutions, deletions, or insertions, e.g., conservative substitutions) relative to one, two, or three hypervariable loops according to Chothia et al. shown in Table 6.

In yet another embodiment, the anti-PD-1 antibody molecule includes at least one, two, three, four, five, or six hypervariable loops (e.g., at least one, two, three, four, five, or six hypervariable loops according to the Chothia definition as set out in Table 6) from the heavy and light chain variable regions of an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 6, or encoded by the nucleotide sequence in Table 6; or at least the amino acids from those hypervariable loops that contact PD-1; or which have at least one amino acid alteration, but not more than two, three or four alterations (e.g., substitutions, deletions, or insertions, e.g., conservative substitutions) relative to one, two, three, four, five or six hypervariable loops according to Chothia et al. shown in Table 6.

In one embodiment, the anti-PD-1 antibody molecule includes all six hypervariable loops (e.g., all six hypervariable loops according to the Chothia definition as set out in Table 6) of an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E, or closely related hypervariable loops, e.g., hypervariable loops which are identical or which have at least one amino acid alteration, but not more than two, three or four alterations (e.g., substitutions, deletions, or insertions, e.g., conservative substitutions); or which have at least one amino acid alteration, but not more than two, three or four alterations (e.g., substitutions, deletions, or insertions, e.g., conservative substitutions) relative to all six hypervariable loops according to Chothia et al. shown in Table 6. In one embodiment, the anti-PD-1 antibody molecule may include any hypervariable loop described herein.

In still another embodiment, the anti-PD-1 antibody molecule includes at least one, two, or three hypervariable loops that have the same canonical structures as the corresponding hypervariable loop of an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E, e.g., the same canonical structures as at least loop 1 and/or loop 2 of the heavy and/or light chain variable domains of an antibody described herein. See, e.g., Chothia et al., (1992) J. Mol. Biol. 227:799-817; Tomlinson et al., (1992) J. Mol. Biol. 227:776-798 for descriptions of hypervariable loop canonical structures. These structures can be determined by inspection of the tables described in these references.

In certain embodiments, the anti-PD-1 antibody molecule includes a combination of CDRs or hypervariable loops defined according to the Kabat et al. and Chothia et al.

In one embodiment, the anti-PD-1 antibody molecule includes at least one, two or three CDRs or hypervariable loops from a heavy chain variable region of an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E, according to the Kabat and Chothia definition (e.g., at least one, two, or three CDRs or hypervariable loops according to the Kabat and Chothia definition as set out in Table 6); or encoded by the nucleotide sequence in Table 6; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs or hypervariable loops according to Kabat and/or Chothia shown in Table 6.

For example, the anti-PD-1 antibody molecule can include VH CDR1 according to Kabat et al. or VH hypervariable loop 1 according to Chothia et al., or a combination thereof, e.g., as shown in Table 6. In one embodiment, the combination of Kabat and Chothia CDR of VH CDR1 comprises the amino acid sequence GYTFTTYWMH (SEQ ID NO: 286), or an amino acid sequence substantially identical thereto (e.g., having at least one amino acid alteration, but not more than two, three or four alterations (e.g., substitutions, deletions, or insertions, e.g., conservative substitutions)). The anti-PD-1 antibody molecule can further include, e.g., VH CDRs 2-3 according to Kabat et al. and VL CDRs 1-3 according to Kabat et al., e.g., as shown in Table 6. Accordingly, in some embodiments, framework regions are defined based on a combination of CDRs defined according to Kabat et al. and hypervariable loops defined according to Chothia et al. For example, the anti-PD-1 antibody molecule can include VH FR1 defined based on VH hypervariable loop 1 according to Chothia et al. and VH FR2 defined based on VH CDRs 1-2 according to Kabat et al., e.g., as shown in Table 6. The anti-PD-1 antibody molecule can further include, e.g., VH FRs 3-4 defined based on VH CDRs 2-3 according to Kabat et al. and VL FRs 1˜4 defined based on VL CDRs 1-3 according to Kabat et al.

The anti-PD-1 antibody molecule can contain any combination of CDRs or hypervariable loops according to the Kabat and Chothia definitions. In one embodiment, the anti-PD-1 antibody molecule includes at least one, two or three CDRs from a light chain variable region of an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E, according to the Kabat and Chothia definition (e.g., at least one, two, or three CDRs according to the Kabat and Chothia definition as set out in Table 6).

In an embodiment, e.g., an embodiment comprising a variable region, a CDR (e.g., Chothia CDR or Kabat CDR), or other sequence referred to herein, e.g., in Table 6, the antibody molecule is a monospecific antibody molecule, a bispecific antibody molecule, or is an antibody molecule that comprises an antigen binding fragment of an antibody, e.g., a half antibody or antigen binding fragment of a half antibody. In certain embodiments the antibody molecule is a bispecific antibody molecule having a first binding specificity for PD-1 and a second binding specificity for TIM-3, LAG-3, CEACAM (e.g., CEACAM-1 and/or CEACAM-5), PD-L1 or PD-L2.

In one embodiment, the anti-PD-1 antibody molecule includes:

(a) a heavy chain variable region (VH) comprising a VHCDR1 amino acid sequence of SEQ ID NO: 140, a VHCDR2 amino acid sequence of SEQ ID NO: 141, and a VHCDR3 amino acid sequence of SEQ ID NO: 139; and a light chain variable region (VL) comprising a VLCDR1 amino acid sequence of SEQ ID NO: 149, a VLCDR2 amino acid sequence of SEQ ID NO: 150, and a VLCDR3 amino acid sequence of SEQ ID NO: 167;

(b) a VH comprising a VHCDR1 amino acid sequence chosen from SEQ ID NO: 137; a VHCDR2 amino acid sequence of SEQ ID NO: 138; and a VHCDR3 amino acid sequence of SEQ ID NO: 139; and a VL comprising a VLCDR1 amino acid sequence of SEQ ID NO: 146, a VLCDR2 amino acid sequence of SEQ ID NO: 147, and a VLCDR3 amino acid sequence of SEQ ID NO: 166;

(c) a VH comprising a VHCDR1 amino acid sequence of SEQ ID NO: 286, a VHCDR2 amino acid sequence of SEQ ID NO: 141, and a VHCDR3 amino acid sequence of SEQ ID NO: 139; and a VL comprising a VLCDR1 amino acid sequence of SEQ ID NO: 149, a VLCDR2 amino acid sequence of SEQ ID NO: 150, and a VLCDR3 amino acid sequence of SEQ ID NO: 167; or

(d) a VH comprising a VHCDR1 amino acid sequence of SEQ ID NO: 286; a VHCDR2 amino acid sequence of SEQ ID NO: 138; and a VHCDR3 amino acid sequence of SEQ ID NO: 139; and a VL comprising a VLCDR1 amino acid sequence of SEQ ID NO: 146, a VLCDR2 amino acid sequence of SEQ ID NO: 147, and a VLCDR3 amino acid sequence of SEQ ID NO: 166.

In one embodiment, the anti-PD-1 antibody molecule comprises a VH comprising a VHCDR1 amino acid sequence of SEQ ID NO: 140, a VHCDR2 amino acid sequence of SEQ ID NO: 141, and a VHCDR3 amino acid sequence of SEQ ID NO: 139; and a VL comprising a VLCDR1 amino acid sequence of SEQ ID NO: 149, a VLCDR2 amino acid sequence of SEQ ID NO: 150, and a VLCDR3 amino acid sequence of SEQ ID NO: 167.

In one embodiment, the anti-PD-1 antibody molecule comprises a VH comprising a VHCDR1 amino acid sequence of SEQ ID NO: 137; a VHCDR2 amino acid sequence of SEQ ID NO: 138; and a VHCDR3 amino acid sequence of SEQ ID NO: 139; and a VL comprising a VLCDR1 amino acid sequence of SEQ ID NO: 146, a VLCDR2 amino acid sequence of SEQ ID NO: 147, and a VLCDR3 amino acid sequence of SEQ ID NO: 166.

In one embodiment, the anti-PD-1 antibody molecule comprises a VH comprising a VHCDR1 amino acid sequence of SEQ ID NO: 286, a VHCDR2 amino acid sequence of SEQ ID NO: 141, and a VHCDR3 amino acid sequence of SEQ ID NO: 139; and a VL comprising a VLCDR1 amino acid sequence of SEQ ID NO: 149, a VLCDR2 amino acid sequence of SEQ ID NO: 150, and a VLCDR3 amino acid sequence of SEQ ID NO: 167.

In one embodiment, the anti-PD-1 antibody molecule comprises a VH comprising a VHCDR1 amino acid sequence of SEQ ID NO: 286; a VHCDR2 amino acid sequence of SEQ ID NO: 138; and a VHCDR3 amino acid sequence of SEQ ID NO: 139; and a VL comprising a VLCDR1 amino acid sequence of SEQ ID NO: 146, a VLCDR2 amino acid sequence of SEQ ID NO: 147, and a VLCDR3 amino acid sequence of SEQ ID NO: 166.

In one embodiment, the antibody molecule is a humanized antibody molecule. In another embodiment, the antibody molecule is a monospecific antibody molecule. In yet another embodiment, the antibody molecule is a bispecific antibody molecule.

In one embodiment, the anti-PD-1 antibody molecule includes:

(i) a heavy chain variable region (VH) including a VHCDR1 amino acid sequence chosen from SEQ ID NO: 137, SEQ ID NO: 140 or SEQ ID NO: 286; a VHCDR2 amino acid sequence of SEQ ID NO: 138; and a VHCDR3 amino acid sequence of SEQ ID NO: 139; and

(ii) a light chain variable region (VL) including a VLCDR1 amino acid sequence of SEQ ID NO: 146, a VLCDR2 amino acid sequence of SEQ ID NO: 147, and a VLCDR3 amino acid sequence of SEQ ID NO: 166.

In another embodiment, the anti-PD-1 antibody molecule includes:

(i) a heavy chain variable region (VH) including a VHCDR1 amino acid sequence chosen from SEQ ID NO: 137, SEQ ID NO: 140 or SEQ ID NO: 286; a VHCDR2 amino acid sequence of SEQ ID NO: 141, and a VHCDR3 amino acid sequence of SEQ ID NO: 139; and

(ii) a light chain variable region (VL) including a VLCDR1 amino acid sequence of SEQ ID NO: 149, a VLCDR2 amino acid sequence of SEQ ID NO: 150, and a VLCDR3 amino acid sequence of SEQ ID NO: 167.

In one embodiment, the anti-PD-1 antibody molecule comprises the VHCDR1 amino acid sequence of SEQ ID NO: 137. In another embodiment, the anti-PD-1 antibody molecule comprises the VHCDR1 amino acid sequence of SEQ ID NO: 140. In yet another embodiment, the anti-PD-1 antibody molecule comprises the VHCDR1 amino acid sequence of SEQ ID NO: 286.

In one embodiment, the light or the heavy chain variable framework (e.g., the region encompassing at least FR1, FR2, FR3, and optionally FR4) of the anti-PD-1 antibody molecule can be chosen from: (a) a light or heavy chain variable framework including at least 80%, 85%, 87% 90%, 92%, 93%, 95%, 97%, 98%, or preferably 100% of the amino acid residues from a human light or heavy chain variable framework, e.g., a light or heavy chain variable framework residue from a human mature antibody, a human germline sequence, or a human consensus sequence; (b) a light or heavy chain variable framework including from 20% to 80%, 40% to 60%, 60% to 90%, or 70% to 95% of the amino acid residues from a human light or heavy chain variable framework, e.g., a light or heavy chain variable framework residue from a human mature antibody, a human germline sequence, or a human consensus sequence; (c) a non-human framework (e.g., a rodent framework); or (d) a non-human framework that has been modified, e.g., to remove antigenic or cytotoxic determinants, e.g., deimmunized, or partially humanized. In one embodiment, the light or heavy chain variable framework region (particularly FR1, FR2 and/or FR3) includes a light or heavy chain variable framework sequence at least 70, 75, 80, 85, 87, 88, 90, 92, 94, 95, 96, 97, 98, 99% identical or identical to the frameworks of a VL or VH segment of a human germline gene.

In certain embodiments, the anti-PD-1 antibody molecule comprises a heavy chain variable domain having at least one, two, three, four, five, six, seven, ten, fifteen, twenty or more changes, e.g., amino acid substitutions or deletions, from an amino acid sequence of BAP049-chi-HC, e.g., the amino acid sequence of the FR region in the entire variable region, e.g., shown in FIGS. 9A-9B of US 2015/0210769A1, or SEQ ID NO: 154, 156, 158 or 160. In one embodiment, the anti-PD-1 antibody molecule comprises a heavy chain variable domain having one or more of: E at position 1, V at position 5, A at position 9, V at position 11, K at position 12, K at position 13, E at position 16, L at position 18, R at position 19, I or V at position 20, G at position 24, I at position 37, A or S at position 40, T at position 41, S at position 42, R at position 43, M or L at position 48, V or F at position 68, T at position 69, I at position 70, S at position 71, A or R at position 72, K or N at position 74, T or K at position 76, S or N at position 77, L at position 79, L at position 81, E or Q at position 82, M at position 83, S or N at position 84, R at position 87, A at position 88, or T at position 91 of amino acid sequence of BAP049-chi-HC, e.g., the amino acid sequence of the FR in the entire variable region, e.g., shown in FIGS. 9A-9B of US 2015/0210769A1, or SEQ ID NO: 154, 156, 158 or 160.

Alternatively, or in combination with the heavy chain substitutions of BAP049-chi-HC described herein, the anti-PD-1 antibody molecule comprises a light chain variable domain having at least one, two, three, four, five, six, seven, ten, fifteen, twenty or more amino acid changes, e.g., amino acid substitutions or deletions, from an amino acid sequence of BAP049-chi-LC, e.g., the amino acid sequence shown in FIGS. 10A-10B of US 2015/0210769A1, or SEQ ID NO: 162 or 164. In one embodiment, the anti-PD-1 antibody molecule comprises a heavy chain variable domain having one or more of: E at position 1, V at position 2, Q at position 3, L at position 4, T at position 7, D or L or A at position 9, F or T at position 10, Q at position 11, S or P at position 12, L or A at position 13, S at position 14, P or L or V at position 15, K at position 16, Q or D at position 17, R at position 18, A at position 19, S at position 20, I or L at position 21, T at position 22, L at position 43, K at position 48, A or S at position 49, R or Q at position 51, Y at position 55, I at position 64, S or P at position 66, S at position 69, Y at position 73, G at position 74, E at position 76, F at position 79, N at position 82, N at position 83, L or I at position 84, E at position 85, S or P at position 86, D at position 87, A or F or I at position 89, T or Y at position 91, F at position 93, or Y at position 102 of the amino acid sequence of BAP049-chi-LC, e.g., the amino acid sequence shown in FIGS. 10A-10B of US 2015/0210769A1, or SEQ ID NO: 162 or 164.

In other embodiments, the anti-PD-1 antibody molecule includes one, two, three, or four heavy chain framework regions (e.g., a VHFW amino acid sequence shown in Table 2 of US 2015/0210769A1, or encoded by the nucleotide sequence shown in Table 2 of US 2015/0210769A1), or a sequence substantially identical thereto.

In yet other embodiments, the anti-PD-1 antibody molecule includes one, two, three, or four light chain framework regions (e.g., a VLFW amino acid sequence shown in Table 2 of US 2015/0210769A1, or encoded by the nucleotide sequence shown in Table 2 of US 2015/0210769A1), or a sequence substantially identical thereto.

In other embodiments, the anti-PD-1 antibody molecule includes one, two, three, or four heavy chain framework regions (e.g., a VHFW amino acid sequence shown in Table 2 of US 2015/0210769A1, or encoded by the nucleotide sequence shown in Table 2 of US 2015/0210769A1), or a sequence substantially identical thereto; and one, two, three, or four light chain framework regions (e.g., a VLFW amino acid sequence shown in Table 2 of US 2015/0210769A1, or encoded by the nucleotide sequence shown in Table 2 of US 2015/0210769A1), or a sequence substantially identical thereto.

In some embodiments, the anti-PD-1 antibody molecule comprises the heavy chain framework region 1 (VHFW1) of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum15, BAP049-hum16, BAP049-Clone-A, BAP049-Clone-B, BAP049-Clone-C, BAP049-Clone-D, or BAP049-Clone-E (e.g., SEQ ID NO: 147 of US 2015/0210769A1). In some embodiments, the antibody molecule comprises the heavy chain framework region 1 (VHFW1) of BAP049-hum14 or BAP049-hum15 (e.g., SEQ ID NO: 151 of US 2015/0210769A1).

In some embodiments, the anti-PD-1 antibody molecule comprises the heavy chain framework region 2 (VHFW2) of BAP049-hum01, BAP049-hum02, BAP049-hum05, BAP049-hum06, BAP049-hum07, BAP049-hum09, BAP049-hum11, BAP049-hum12, BAP049-hum13, BAP049-Clone-A, BAP049-Clone-B, BAP049-Clone-C, or BAP049-Clone-E (e.g., SEQ ID NO: 153 of US 2015/0210769A1). In some embodiments, the antibody molecule comprises the heavy chain framework region 2 (VHFW2) of BAP049-hum03, BAP049-hum04, BAP049-hum08, BAP049-hum10, BAP049-hum14, BAP049-hum15, or BAP049-Clone-D (e.g., SEQ ID NO: 157 of US 2015/0210769A1). In some embodiments, the antibody molecule comprises the heavy chain framework region 2 (VHFW2) of BAP049-hum16 (e.g., SEQ ID NO: 160 of US 2015/0210769A1).

In some embodiments, the anti-PD-1 antibody molecule comprises the heavy chain framework region 3 (VHFW3) of BAP049-hum01, BAP049-hum02, BAP049-hum05, BAP049-hum06, BAP049-hum07, BAP049-hum09, BAP049-hum11, BAP049-hum12, BAP049-hum13, BAP049-Clone-A, BAP049-Clone-B, BAP049-Clone-C, or BAP049-Clone-E (e.g., SEQ ID NO: 162 of US 2015/0210769A1). In some embodiments, the antibody molecule comprises the heavy chain framework region 3 (VHFW3) of BAP049-hum03, BAP049-hum04, BAP049-hum08, BAP049-hum10, BAP049-hum14, BAP049-hum15, BAP049-hum16, or BAP049-Clone-D (e.g., SEQ ID NO: 166 of US 2015/0210769A1).

In some embodiments, the anti-PD-1 antibody molecule comprises the heavy chain framework region 4 (VHFW4) of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E (e.g., SEQ ID NO: 169 of US 2015/0210769A1).

In some embodiments, the anti-PD-1 antibody molecule comprises the light chain framework region 1 (VLFW1) of BAP049-hum08, BAP049-hum09, BAP049-hum15, BAP049-hum16, or BAP049-Clone-C(e.g., SEQ ID NO: 174 of US 2015/0210769A1). In some embodiments, the antibody molecule comprises the light chain framework region 1 (VLFW1) of BAP049-hum01, BAP049-hum04, BAP049-hum05, BAP049-hum07, BAP049-hum10, BAP049-hum11, BAP049-hum14, BAP049-Clone-A, BAP049-Clone-B, BAP049-Clone-D, or BAP049-Clone-E (e.g., SEQ ID NO: 177 of US 2015/0210769A1). In some embodiments, the antibody molecule comprises the light chain framework region 1 (VLFW1) of BAP049-hum06 (e.g., SEQ ID NO: 181 of US 2015/0210769A1). In some embodiments, the antibody molecule comprises the light chain framework region 1 (VLFW1) of BAP049-hum13 (e.g., SEQ ID NO: 183 of US 2015/0210769A1). In some embodiments, the antibody molecule comprises the light chain framework region 1 (VLFW1) of BAP049-hum02, BAP049-hum03, or BAP049-hum12 (e.g., SEQ ID NO: 185 of US 2015/0210769A1).

In some embodiments, the anti-PD-1 antibody molecule comprises the light chain framework region 2 (VLFW2) of BAP049-hum01, BAP049-hum02, BAP049-hum03, BAP049-hum06, BAP049-hum08, BAP049-hum09, BAP049-hum10, BAP049-hum11, BAP049-hum14, BAP049-hum15, BAP049-hum16, BAP049-Clone-A, BAP049-Clone-B, BAP049-Clone-D, or BAP049-Clone-E (e.g., SEQ ID NO: 187 of US 2015/0210769A1). In some embodiments, the antibody molecule comprises the light chain framework region 2 (VLFW2) of BAP049-hum04, BAP049-hum05, BAP049-hum07, BAP049-hum13, or BAP049-Clone-C(e.g., SEQ ID NO: 191 of US 2015/0210769A1). In some embodiments, the antibody molecule comprises the light chain framework region 2 (VLFW2) of BAP049-hum12 (e.g., SEQ ID NO: 194 of US 2015/0210769A1).

In some embodiments, the anti-PD-1 antibody molecule comprises the light chain framework region 3 (VLFW3) of BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-C, BAP049-Clone-D, or BAP049-Clone-E (e.g., SEQ ID NO: 196 of US 2015/0210769A1). In some embodiments, the antibody molecule comprises the light chain framework region 3 (VLFW3) of BAP049-hum02 or BAP049-hum03 (e.g., SEQ ID NO: 200 of US 2015/0210769A1). In some embodiments, the antibody molecule comprises the light chain framework region 3 (VLFW3) of BAP049-hum01 or BAP049-Clone-A (e.g., SEQ ID NO: 202 of US 2015/0210769A1). In some embodiments, the antibody molecule comprises the light chain framework region 3 (VLFW3) of BAP049-hum04, BAP049-hum05, or BAP049-Clone-B (e.g., SEQ ID NO: 205 of US 2015/0210769A1).

In some embodiments, the anti-PD-1 antibody molecule comprises the light chain framework region 4 (VLFW4) of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E (e.g., SEQ ID NO: 208 of US 2015/0210769A1).

In some embodiments, the anti-PD-1 antibody molecule comprises the heavy chain framework regions 1-3 of BAP049-hum01, BAP049-hum02, BAP049-hum05, BAP049-hum06, BAP-hum07, BAP049-hum09, BAP049-hum11, BAP049-hum12, BAP049-hum13, BAP049-Clone-A, BAP049-Clone-B, BAP049-Clone-C, or BAP049-Clone-E (e.g., SEQ ID NO: 147 (VHFW1), SEQ ID NO: 153 (VHFW2), and SEQ ID NO: 162 (VHFW3) of US 2015/0210769A1). In some embodiments, the antibody molecule comprises the heavy chain framework regions 1-3 of BAP049-hum03, BAP049-hum04, BAP049-hum08, BAP049-hum10, or BAP049-Clone-D (e.g., SEQ ID NO: 147 (VHFW1), SEQ ID NO: 157 (VHFW2), and SEQ ID NO: 166 (VHFW3) of US 2015/0210769A1). In some embodiments, the antibody molecule comprises the heavy chain framework regions 1-3 of BAP049-hum14 or BAP049-hum15 (e.g., SEQ ID NO: 151 (VHFW1), SEQ ID NO: 157 (VHFW2), and SEQ ID NO: 166 (VHFW3) of US 2015/0210769A1). In some embodiments, the antibody molecule comprises the heavy chain framework regions 1-3 of BAP049-hum16 (e.g., SEQ ID NO: 147 (VHFW1), SEQ ID NO: 160 (VHFW2), and SEQ ID NO: 166 (VHFW3) of US 2015/0210769A1). In some embodiments, the antibody molecule further comprises the heavy chain framework region 4 (VHFW4) of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E (e.g., SEQ ID NO: 169 of US 2015/0210769A1).

In some embodiments, the anti-PD-1 antibody molecule comprises the light chain framework regions 1-3 of BAP049-hum01 or BAP049-Clone-A (e.g., SEQ ID NO: 177 (VLFW1), SEQ ID NO: 187 (VLFW2), and SEQ ID NO: 202 (VLFW3) of US 2015/0210769A1). In some embodiments, the antibody molecule comprises the light chain framework regions 1-3 of BAP049-hum02 or BAP049-hum03 (e.g., SEQ ID NO: 185 (VLFW1), SEQ ID NO: 187 (VLFW2), and SEQ ID NO: 200 (VLFW3) of US 2015/0210769A1). In some embodiments, the antibody molecule comprises the light chain framework regions 1-3 of BAP049-hum04, BAP049-hum05, or BAP049-Clone-B (e.g., SEQ ID NO: 177 (VLFW1), SEQ ID NO: 191 (VLFW2), and SEQ ID NO: 205 (VLFW3) of US 2015/0210769A1). In some embodiments, the antibody molecule comprises the light chain framework regions 1-3 of BAP049-hum06 (e.g., SEQ ID NO: 181 (VLFW1), SEQ ID NO: 187 (VLFW2), and SEQ ID NO: 196 (VLFW3) of US 2015/0210769A1). In some embodiments, the antibody molecule comprises the light chain framework regions 1-3 of BAP049-hum07 (e.g., SEQ ID NO: 177 (VLFW1), SEQ ID NO: 191 (VLFW2), and SEQ ID NO: 196 (VLFW3) of US 2015/0210769A1). In some embodiments, the antibody molecule comprises the light chain framework regions 1-3 of BAP049-hum08, BAP049-hum09, BAP049-hum15, BAP049-hum16, or BAP049-Clone-C(e.g., SEQ ID NO: 174 (VLFW1), SEQ ID NO: 187 (VLFW2), and SEQ ID NO: 196 (VLFW3) of US 2015/0210769A1). In some embodiments, the antibody molecule comprises the light chain framework regions 1-3 of BAP049-hum10, BAP049-hum11, BAP049-hum14, BAP049-Clone-D, or BAP049-Clone-E (e.g., SEQ ID NO: 177 (VLFW1), SEQ ID NO: 187 (VLFW2), and SEQ ID NO: 196 (VLFW3) of US 2015/0210769A1). In some embodiments, the antibody molecule comprises the light chain framework regions 1-3 of BAP049-hum12 (e.g., SEQ ID NO: 185 (VLFW1), SEQ ID NO: 194 (VLFW2), and SEQ ID NO: 196 (VLFW3) of US 2015/0210769A1). In some embodiments, the antibody molecule comprises the light chain framework regions 1-3 of BAP049-hum13 (e.g., SEQ ID NO: 183 (VLFW1), SEQ ID NO: 191 (VLFW2), and SEQ ID NO: 196 (VLFW3) of US 2015/0210769A1). In some embodiments, the antibody molecule further comprises the light chain framework region 4 (VLFW4) of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E (e.g., SEQ ID NO: 208 of US 2015/0210769A1).

In some embodiments, the anti-PD-1 antibody molecule comprises the heavy chain framework regions 1-3 of BAP049-hum01 or BAP049-Clone-A (e.g., SEQ ID NO: 147 (VHFW1), SEQ ID NO: 153 (VHFW2), and SEQ ID NO: 162 (VHFW3) of US 2015/0210769A1) and the light chain framework regions 1-3 of BAP049-hum01 or BAP049-Clone-A (e.g., SEQ ID NO: 177 (VLFW1), SEQ ID NO: 187 (VLFW2), and SEQ ID NO: 202 (VLFW3) of US 2015/0210769A1).

In some embodiments, the anti-PD-1 antibody molecule comprises the heavy chain framework regions 1-3 of BAP049-hum02 (e.g., SEQ ID NO: 147 (VHFW1), SEQ ID NO: 153 (VHFW2), and SEQ ID NO: 162 (VHFW3) of US 2015/0210769A1) and the light chain framework regions 1-3 of BAP049-hum02 (e.g., SEQ ID NO: 185 (VLFW1), SEQ ID NO: 187 (VLFW2), and SEQ ID NO: 200 (VLFW3) of US 2015/0210769A1).

In some embodiments, the anti-PD-1 antibody molecule comprises the heavy chain framework regions 1-3 of BAP049-hum03 (e.g., SEQ ID NO: 147 (VHFW1), SEQ ID NO: 157 (VHFW2), and SEQ ID NO: 166 (VHFW3) of US 2015/0210769A1) and the light chain framework regions 1-3 of BAP049-hum03 (e.g., SEQ ID NO: 185 (VLFW1), SEQ ID NO: 187 (VLFW2), and SEQ ID NO: 200 (VLFW3) of US 2015/0210769A1).

In some embodiments, the anti-PD-1 antibody molecule comprises the heavy chain framework regions 1-3 of BAP049-hum04 (e.g., SEQ ID NO: 147 (VHFW1), SEQ ID NO: 157 (VHFW2), and SEQ ID NO: 166 (VHFW3) of US 2015/0210769A1) and the light chain framework regions 1-3 of BAP049-hum04 (e.g., SEQ ID NO: 177 (VLFW1), SEQ ID NO: 191 (VLFW2), and SEQ ID NO: 205 (VLFW3) of US 2015/0210769A1).

In some embodiments, the anti-PD-1 antibody molecule comprises the heavy chain framework regions 1-3 of BAP049-hum05 or BAP049-Clone-B (e.g., SEQ ID NO: 147 (VHFW1), SEQ ID NO: 153 (VHFW2), and SEQ ID NO: 162 (VHFW3) of US 2015/0210769A1) and the light chain framework regions 1-3 of BAP049-hum05 or BAP049-Clone-B (e.g., SEQ ID NO: 177 (VLFW1), SEQ ID NO: 191 (VLFW2), and SEQ ID NO: 205 (VLFW3) of US 2015/0210769A1).

In some embodiments, the anti-PD-1 antibody molecule comprises the heavy chain framework regions 1-3 of BAP049-hum06 (e.g., SEQ ID NO: 147 (VHFW1), SEQ ID NO: 153 (VHFW2), and SEQ ID NO: 162 (VHFW3) of US 2015/0210769A1) and the light chain framework regions 1-3 of BAP049-hum06 (e.g., SEQ ID NO: 181 (VLFW1), SEQ ID NO: 187 (VLFW2), and SEQ ID NO: 196 (VLFW3) of US 2015/0210769A1).

In some embodiments, the anti-PD-1 antibody molecule comprises the heavy chain framework regions 1-3 of BAP049-hum07 (e.g., SEQ ID NO: 147 (VHFW1), SEQ ID NO: 153 (VHFW2), and SEQ ID NO: 162 (VHFW3) of US 2015/0210769A1) and the light chain framework regions 1-3 of BAP049-hum07 (e.g., SEQ ID NO: 177 (VLFW1), SEQ ID NO: 191 (VLFW2), and SEQ ID NO: 196 (VLFW3) of US 2015/0210769A1).

In some embodiments, the anti-PD-1 antibody molecule comprises the heavy chain framework regions 1-3 of BAP049-hum08 (e.g., SEQ ID NO: 147 (VHFW1), SEQ ID NO: 157 (VHFW2), and SEQ ID NO: 166 (VHFW3) of US 2015/0210769A1) and the light chain framework regions 1-3 of BAP049-hum08 (e.g., SEQ ID NO: 174 (VLFW1), SEQ ID NO: 187 (VLFW2), and SEQ ID NO: 196 (VLFW3) of US 2015/0210769A1).

In some embodiments, the anti-PD-1 antibody molecule comprises the heavy chain framework regions 1-3 of BAP049-hum09 or BAP049-Clone-C(e.g., SEQ ID NO: 147 (VHFW1), SEQ ID NO: 153 (VHFW2), and SEQ ID NO: 162 (VHFW3) of US 2015/0210769A1) and the light chain framework regions 1-3 of BAP049-hum09 or BAP049-Clone-C(e.g., SEQ ID NO: 174 (VLFW1), SEQ ID NO: 187 (VLFW2), and SEQ ID NO: 196 (VLFW3) of US 2015/0210769A1).

In some embodiments, the anti-PD-1 antibody molecule comprises the heavy chain framework regions 1-3 of BAP049-hum10 or BAP049-Clone-D (e.g., SEQ ID NO: 147 (VHFW1), SEQ ID NO: 157 (VHFW2), and SEQ ID NO: 166 (VHFW3) of US 2015/0210769A1) and the light chain framework regions 1-3 of BAP049-hum10 or BAP049-Clone-D (e.g., SEQ ID NO: 177 (VLFW1), SEQ ID NO: 187 (VLFW2), and SEQ ID NO: 196 (VLFW3) of US 2015/0210769A1).

In some embodiments, the anti-PD-1 antibody molecule comprises the heavy chain framework regions 1-3 of BAP049-hum11 or BAP049-Clone-E (e.g., SEQ ID NO: 147 (VHFW1), SEQ ID NO: 153 (VHFW2), and SEQ ID NO: 162 (VHFW3) of US 2015/0210769A1) and the light chain framework regions 1-3 of BAP049-hum11 or BAP049-Clone-E (e.g., SEQ ID NO: 177 (VLFW1), SEQ ID NO: 187 (VLFW2), and SEQ ID NO: 196 (VLFW3) of US 2015/0210769A1).

In some embodiments, the anti-PD-1 antibody molecule comprises the heavy chain framework regions 1-3 of BAP049-hum12 (e.g., SEQ ID NO: 147 (VHFW1), SEQ ID NO: 153 (VHFW2), and SEQ ID NO: 162 (VHFW3) of US 2015/0210769A1) and the light chain framework regions 1-3 of BAP049-hum12 (e.g., SEQ ID NO: 185 (VLFW1), SEQ ID NO: 194 (VLFW2), and SEQ ID NO: 196 (VLFW3) of US 2015/0210769A1).

In some embodiments, the anti-PD-1 antibody molecule comprises the heavy chain framework regions 1-3 of BAP049-hum13 (e.g., SEQ ID NO: 147 (VHFW1), SEQ ID NO: 153 (VHFW2), and SEQ ID NO: 162 (VHFW3) of US 2015/0210769A1) and the light chain framework regions 1-3 of BAP049-hum13 (e.g., SEQ ID NO: 183 (VLFW1), SEQ ID NO: 191 (VLFW2), and SEQ ID NO: 196 (VLFW3) of US 2015/0210769A1).

In some embodiments, the anti-PD-1 antibody molecule comprises the heavy chain framework regions 1-3 of BAP049-hum14 (e.g., SEQ ID NO: 151 (VHFW1), SEQ ID NO: 157 (VHFW2), and SEQ ID NO: 166 (VHFW3) of US 2015/0210769A1) and the light chain framework regions 1-3 of BAP049-hum14 (e.g., SEQ ID NO: 177 (VLFW1), SEQ ID NO: 187 (VLFW2), and SEQ ID NO: 196 (VLFW3) of US 2015/0210769A1).

In some embodiments, the anti-PD-1 antibody molecule comprises the heavy chain framework regions 1-3 of BAP049-hum15 (e.g., SEQ ID NO: 151 (VHFW1), SEQ ID NO: 157 (VHFW2), and SEQ ID NO: 166 (VHFW3) of US 2015/0210769A1) and the light chain framework regions 1-3 of BAP049-hum15 (e.g., SEQ ID NO: 174 (VLFW1), SEQ ID NO: 187 (VLFW2), and SEQ ID NO: 196 (VLFW3) of US 2015/0210769A1).

In some embodiments, the anti-PD-1 antibody molecule comprises the heavy chain framework regions 1-3 of BAP049-hum16 (e.g., SEQ ID NO: 147 (VHFW1), SEQ ID NO: 160 (VHFW2), and SEQ ID NO: 166 (VHFW3) of US 2015/0210769A1) and the light chain framework regions 1-3 of BAP049-hum16 (e.g., SEQ ID NO: 174 (VLFW1), SEQ ID NO: 187 (VLFW2), and SEQ ID NO: 196 (VLFW3) of US 2015/0210769A1).

In some embodiments, the anti-PD-1 antibody molecule further comprises the heavy chain framework region 4 (VHFW4) of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E (e.g., SEQ ID NO: 169 of US 2015/0210769A1) and the light chain framework region 4 (VLFW4) of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E (e.g., SEQ ID NO: 208 of US 2015/0210769A1).

In some embodiments, the anti-PD-1 antibody molecule comprises a heavy chain framework region having a combination of framework regions FW1, FW2 and FW3 as show in FIG. 5 or 7 of US 2015/0210769A1. In other embodiment, the antibody molecule comprises a light chain framework region having a combination of framework regions FW1, FW2 and FW3 as show in FIG. 5 or 7 of US 2015/0210769A1. In yet other embodiments, the antibody molecule comprises a heavy chain framework region having a combination of framework regions FW1, FW2 and FW3 as show in FIG. 5 or 7 of US 2015/0210769A1, and a light chain framework region having a combination of framework regions FW1, FW2 and FW3 as shown in FIG. 5 or 7 of US 2015/0210769A1.

In one embodiment, the heavy or light chain variable domain, or both, of the anti-PD-1 antibody molecule includes an amino acid sequence, which is substantially identical to an amino acid disclosed herein, e.g., at least 80%, 85%, 90%, 92%, 95%, 97%, 98%, 99% or higher identical to a variable region of an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-Clone-A, BAP049-Clone-B, BAP049-Clone-C, BAP049-Clone-D, or BAP049-Clone-E; or as described in Table 6, or encoded by the nucleotide sequence in Table 6; or which differs at least 1 or 5 residues, but less than 40, 30, 20, or 10 residues, from a variable region of an antibody described herein.

In one embodiment, the heavy or light chain variable region, or both, of the anti-PD-1 antibody molecule includes an amino acid sequence encoded by a nucleic acid sequence described herein or a nucleic acid that hybridizes to a nucleic acid sequence described herein (e.g., a nucleic acid sequence as shown in Tables 1 and 2 of US 2015/0210769A1, or Table 6 herein) or its complement, e.g., under low stringency, medium stringency, or high stringency, or other hybridization condition described herein.

In another embodiment, the anti-PD-1 antibody molecule comprises at least one, two, three, or four antigen-binding regions, e.g., variable regions, having an amino acid sequence as set forth in Table 6, or a sequence substantially identical thereto (e.g., a sequence at least about 85%, 90%, 95%, 99% or more identical thereto, or which differs by no more than 1, 2, 5, 10, or 15 amino acid residues from the sequences shown in Table 6. In another embodiment, the anti-PD-1 antibody molecule includes a VH and/or VL domain encoded by a nucleic acid having a nucleotide sequence as set forth in Table 6, or a sequence substantially identical thereto (e.g., a sequence at least about 85%, 90%, 95%, 99% or more identical thereto, or which differs by no more than 3, 6, 15, 30, or 45 nucleotides from the sequences shown in Table 6.

In yet another embodiment, the anti-PD-1 antibody molecule comprises at least one, two, or three CDRs from a heavy chain variable region having an amino acid sequence as set forth in Table 6, or a sequence substantially homologous thereto (e.g., a sequence at least about 85%, 90%, 95%, 99% or more identical thereto, and/or having one, two, three or more substitutions, insertions or deletions, e.g., conserved substitutions). In yet another embodiment, the anti-PD-1 antibody molecule comprises at least one, two, or three CDRs from a light chain variable region having an amino acid sequence as set forth in Table 6, or a sequence substantially homologous thereto (e.g., a sequence at least about 85%, 90%, 95%, 99% or more identical thereto, and/or having one, two, three or more substitutions, insertions or deletions, e.g., conserved substitutions). In yet another embodiment, the anti-PD-1 antibody molecule comprises at least one, two, three, four, five or six CDRs from heavy and light chain variable regions having an amino acid sequence as set forth in Table 6), or a sequence substantially homologous thereto (e.g., a sequence at least about 85%, 90%, 95%, 99% or more identical thereto, and/or having one, two, three or more substitutions, insertions or deletions, e.g., conserved substitutions).

In one embodiment, the anti-PD-1 antibody molecule comprises at least one, two, or three CDRs and/or hypervariable loops from a heavy chain variable region having an amino acid sequence of an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E, as summarized in Table 6, or a sequence substantially identical thereto (e.g., a sequence at least about 85%, 90%, 95%, 99% or more identical thereto, and/or having one, two, three or more substitutions, insertions or deletions, e.g., conserved substitutions). In another embodiment, the anti-PD-1 antibody molecule comprises at least one, two, or three CDRs and/or hypervariable loops from a light chain variable region having an amino acid sequence of an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E, as summarized in Table 6, or a sequence substantially identical thereto (e.g., a sequence at least about 85%, 90%, 95%, 99% or more identical thereto, and/or having one, two, three or more substitutions, insertions or deletions, e.g., conserved substitutions). In one embodiment, the anti-PD-1 antibody molecule comprises all six CDRs and/or hypervariable loops described herein, e.g., described in Table 6.

In one embodiment, the anti-PD-1 antibody molecule has a variable region that is identical in sequence, or which differs by 1, 2, 3, or 4 amino acids from a variable region described herein (e.g., an FR region disclosed herein).

In one embodiment, the anti-PD-1 antibody molecule is a full antibody or fragment thereof (e.g., a Fab, F(ab′)2, Fv, or a single chain Fv fragment (scFv)). In certain embodiments, the anti-PD-1 antibody molecule is a monoclonal antibody or an antibody with single specificity. The anti-PD-1 antibody molecule can also be a humanized, chimeric, camelid, shark, or an in vitro-generated antibody molecule. In one embodiment, the anti-PD-1 antibody molecule thereof is a humanized antibody molecule. The heavy and light chains of the anti-PD-1 antibody molecule can be full-length (e.g., an antibody can include at least one, and preferably two, complete heavy chains, and at least one, and preferably two, complete light chains) or can include an antigen-binding fragment (e.g., a Fab, F(ab′)2, Fv, a single chain Fv fragment, a single domain antibody, a diabody (dAb), a bivalent antibody, or bispecific antibody or fragment thereof, a single domain variant thereof, or a camelid antibody).

In yet other embodiments, the anti-PD-1 antibody molecule has a heavy chain constant region (Fc) chosen from, e.g., the heavy chain constant regions of IgG1, IgG2, IgG3, IgG4, IgM, IgA1, IgA2, IgD, and IgE; particularly, chosen from, e.g., the heavy chain constant regions of IgG1, IgG2, IgG3, and IgG4, more particularly, the heavy chain constant region of IgG1 or IgG2 (e.g., human IgG1, IgG2 or IgG4). In one embodiment, the heavy chain constant region is human IgG1. In another embodiment, the anti-PD-1 antibody molecule has a light chain constant region chosen from, e.g., the light chain constant regions of kappa or lambda, preferably kappa (e.g., human kappa). In one embodiment, the constant region is altered, e.g., mutated, to modify the properties of the anti-PD-1 antibody molecule (e.g., to increase or decrease one or more of: Fc receptor binding, antibody glycosylation, the number of cysteine residues, effector cell function, or complement function). For example, the constant region is mutated at positions 296 (M to Y), 298 (S to T), 300 (T to E), 477 (H to K) and 478 (N to F) to alter Fc receptor binding (e.g., the mutated positions correspond to positions 132 (M to Y), 134 (S to T), 136 (T to E), 313 (H to K) and 314 (N to F) of SEQ ID NOs: 212 or 214; or positions 135 (M to Y), 137 (S to T), 139 (T to E), 316 (H to K) and 317 (N to F) of SEQ ID NOs: 215, 216, 217 or 218). In another embodiment, the heavy chain constant region of an IgG4, e.g., a human IgG4, is mutated at position 228 according to EU numbering (e.g., S to P), e.g., as shown in Table 3 of US 2015/0210769A1. In certain embodiments, the anti-PD-1 antibody molecules comprises a human IgG4 mutated at position 228 according to EU numbering (e.g., S to P), e.g., as shown in Table 3 of US 2015/0210769A1; and a kappa light chain constant region, e.g., as shown in Table 3 of US 2015/0210769A1. In still another embodiment, the heavy chain constant region of an IgG1, e.g., a human IgG1, is mutated at one or more of position 297 according to EU numbering (e.g., N to A), position 265 according to EU numbering (e.g., D to A), position 329 according to EU numbering (e.g., P to A), position 234 according to EU numbering (e.g., L to A), or position 235 according to EU numbering (e.g., L to A), e.g., as shown in Table 3 of US 2015/0210769A1. In certain embodiments, the anti-PD-1 antibody molecules comprises a human IgG1 mutated at one or more of the aforesaid positions, e.g., as shown in Table 3 of US 2015/0210769A1; and a kappa light chain constant region, e.g., as shown in Table 3 of US 2015/0210769A1.

In one embodiment, the anti-PD-1 antibody molecule is isolated or recombinant.

In one embodiment, the anti-PD-1 antibody molecule is a humanized antibody molecule.

In one embodiment, the anti-PD-1 antibody molecule has a risk score based on T cell epitope analysis of less than 700, 600, 500, 400 or less.

In one embodiment, the anti-PD-1 antibody molecule is a humanized antibody molecule and has a risk score based on T cell epitope analysis of 300 to 700, 400 to 650, 450 to 600, or a risk score as described herein.

In one embodiment, the anti-PD-1 antibody molecule includes:

(a) a heavy chain variable region (VH) comprising a VHCDR1 amino acid sequence of SEQ ID NO: 140, a VHCDR2 amino acid sequence of SEQ ID NO: 141, and a VHCDR3 amino acid sequence of SEQ ID NO: 139; and a light chain variable region (VL) comprising a VLCDR1 amino acid sequence of SEQ ID NO: 149, a VLCDR2 amino acid sequence of SEQ ID NO: 150, and a VLCDR3 amino acid sequence of SEQ ID NO: 167;

(b) a VH comprising a VHCDR1 amino acid sequence chosen from SEQ ID NO: 137; a VHCDR2 amino acid sequence of SEQ ID NO: 138; and a VHCDR3 amino acid sequence of SEQ ID NO: 139; and a VL comprising a VLCDR1 amino acid sequence of SEQ ID NO: 146, a VLCDR2 amino acid sequence of SEQ ID NO: 147, and a VLCDR3 amino acid sequence of SEQ ID NO: 166;

(c) a VH comprising a VHCDR1 amino acid sequence of SEQ ID NO: 286, a VHCDR2 amino acid sequence of SEQ ID NO: 141, and a VHCDR3 amino acid sequence of SEQ ID NO: 139; and a VL comprising a VLCDR1 amino acid sequence of SEQ ID NO: 149, a VLCDR2 amino acid sequence of SEQ ID NO: 150, and a VLCDR3 amino acid sequence of SEQ ID NO: 167; or

(d) a VH comprising a VHCDR1 amino acid sequence of SEQ ID NO: 286; a VHCDR2 amino acid sequence of SEQ ID NO: 138; and a VHCDR3 amino acid sequence of SEQ ID NO: 139; and a VL comprising a VLCDR1 amino acid sequence of SEQ ID NO: 146, a VLCDR2 amino acid sequence of SEQ ID NO: 147, and a VLCDR3 amino acid sequence of SEQ ID NO: 166.

In certain embodiments, the anti-PD-1 antibody molecule comprises:

(i) a heavy chain variable region (VH) comprising a VHCDR1 amino acid sequence chosen from SEQ ID NO: 137, SEQ ID NO: 140 or SEQ ID NO: 286; a VHCDR2 amino acid sequence of SEQ ID NO: 138; and a VHCDR3 amino acid sequence of SEQ ID NO: 139; and

(ii) a light chain variable region (VL) comprising a VLCDR1 amino acid sequence of SEQ ID NO: 146, a VLCDR2 amino acid sequence of SEQ ID NO: 147, and a VLCDR3 amino acid sequence of SEQ ID NO: 166.

In other embodiments, the anti-PD-1 antibody molecule comprises:

(i) a heavy chain variable region (VH) comprising a VHCDR1 amino acid sequence chosen from SEQ ID NO: 137, SEQ ID NO: 140 or SEQ ID NO: 286; a VHCDR2 amino acid sequence of SEQ ID NO: 141, and a VHCDR3 amino acid sequence of SEQ ID NO: 139; and (ii) a light chain variable region (VL) comprising a VLCDR1 amino acid sequence of SEQ ID NO: 149, a VLCDR2 amino acid sequence of SEQ ID NO: 150, and a VLCDR3 amino acid sequence of SEQ ID NO: 167.

In embodiments of the aforesaid antibody molecules, the VHCDR1 comprises the amino acid sequence of SEQ ID NO: 137. In other embodiments, the VHCDR1 comprises the amino acid sequence of SEQ ID NO: 140. In yet other embodiments, the VHCDR1 amino acid sequence of SEQ ID NO: 286.

In embodiments, the aforesaid antibody molecules have a heavy chain variable region comprising at least one framework (FW) region comprising the amino acid sequence of any of SEQ ID NOs: 147, 151, 153, 157, 160, 162, 166, or 169 of US 2015/0210769A1, or an amino acid sequence at least 90% identical thereto, or having no more than two amino acid substitutions, insertions or deletions compared to the amino acid sequence of any of SEQ ID NOs: 147, 151, 153, 157, 160, 162, 166, or 169 of US 2015/0210769A1.

In other embodiments, the aforesaid antibody molecules have a heavy chain variable region comprising at least one framework region comprising the amino acid sequence of any of SEQ ID NOs: 147, 151, 153, 157, 160, 162, 166, or 169 of US 2015/0210769A1.

In yet other embodiments, the aforesaid antibody molecules have a heavy chain variable region comprising at least two, three, or four framework regions comprising the amino acid sequences of any of SEQ ID NOs: 147, 151, 153, 157, 160, 162, 166, or 169 of US 2015/0210769A1.

In other embodiments, the aforesaid antibody molecules comprise a VHFW1 amino acid sequence of SEQ ID NO: 147 or 151 of US 2015/0210769A1, a VHFW2 amino acid sequence of SEQ ID NO: 153, 157, or 160 of US 2015/0210769A1, and a VHFW3 amino acid sequence of SEQ ID NO: 162 or 166 of US 2015/0210769A1, and, optionally, further comprising a VHFW4 amino acid sequence of SEQ ID NO: 169 of US 2015/0210769A1.

In other embodiments, the aforesaid antibody molecules have a light chain variable region comprising at least one framework region comprising the amino acid sequence of any of SEQ ID NOs: 174, 177, 181, 183, 185, 187, 191, 194, 196, 200, 202, 205, or 208 of US 2015/0210769A1, or an amino acid sequence at least 90% identical thereto, or having no more than two amino acid substitutions, insertions or deletions compared to the amino acid sequence of any of 174, 177, 181, 183, 185, 187, 191, 194, 196, 200, 202, 205, or 208 of US 2015/0210769A1.

In other embodiments, the aforesaid antibody molecules have a light chain variable region comprising at least one framework region comprising the amino acid sequence of any of SEQ ID NOs: 174, 177, 181, 183, 185, 187, 191, 194, 196, 200, 202, 205, or 208 of US 2015/0210769A1.

In other embodiments, the aforesaid antibody molecules have a light chain variable region comprising at least two, three, or four framework regions comprising the amino acid sequences of any of SEQ ID NOs: 174, 177, 181, 183, 185, 187, 191, 194, 196, 200, 202, 205, or 208 of US 2015/0210769A1.

In other embodiments, the aforesaid antibody molecules comprise a VLFW1 amino acid sequence of SEQ ID NO: 174, 177, 181, 183, or 185 of US 2015/0210769A1, a VLFW2 amino acid sequence of SEQ ID NO: 187, 191, or 194 of US 2015/0210769A1, and a VLFW3 amino acid sequence of SEQ ID NO: 196, 200, 202, or 205 of US 2015/0210769A1, and, optionally, further comprising a VLFW4 amino acid sequence of SEQ ID NO: 208 of US 2015/0210769A1.

In other embodiments, the aforesaid antibodies comprise a heavy chain variable domain comprising an amino acid sequence at least 85% identical to any of SEQ ID NOs: 172, 184, 216, or 220.

In other embodiments, the aforesaid antibody molecules comprise a heavy chain variable domain comprising the amino acid sequence of SEQ ID NO: 172, 184, 216, or 220.

In other embodiments, the aforesaid antibody molecules comprise a light chain variable domain comprising an amino acid sequence at least 85% identical to any of SEQ ID NOs: 176, 180, 188, 192, 196, 200, 204, 208, or 212.

In other embodiments, the aforesaid antibody molecules comprise a light chain variable domain comprising the amino acid sequence of SEQ ID NO: 176, 180, 188, 192, 196, 200, 204, 208, or 212.

In other embodiments, the aforesaid antibody molecules comprise a heavy chain variable domain comprising the amino acid sequence of SEQ ID NO: 172.

In other embodiments, the aforesaid antibody molecules comprise a heavy chain comprising the amino acid sequence of SEQ ID NO: 174.

In other embodiments, the aforesaid antibody molecules comprise a heavy chain comprising the amino acid sequence of SEQ ID NO: 225.

In other embodiments, the aforesaid antibody molecules comprise a heavy chain variable domain comprising the amino acid sequence of SEQ ID NO: 184.

In other embodiments, the aforesaid antibody molecules comprise a heavy chain comprising the amino acid sequence of SEQ ID NO: 186 or SEQ ID NO: 236.

In other embodiments, the aforesaid antibody molecules comprise a heavy chain variable domain comprising the amino acid sequence of SEQ ID NO: 216.

In other embodiments, the aforesaid antibody molecules comprise a heavy chain comprising the amino acid sequence of SEQ ID NO: 218.

In other embodiments, the aforesaid antibody molecules comprise a heavy chain variable domain comprising the amino acid sequence of SEQ ID NO: 220.

In other embodiments, the aforesaid antibody molecules comprise a heavy chain comprising the amino acid sequence of SEQ ID NO: 222.

In other embodiments, the aforesaid antibody molecules comprise a light chain variable domain comprising the amino acid sequence of SEQ ID NO: 176.

In other embodiments, the aforesaid antibody molecules comprise a light chain comprising the amino acid sequence of SEQ ID NO: 178.

In other embodiments, the aforesaid antibody molecules comprise a light chain variable domain comprising the amino acid sequence of SEQ ID NO: 180.

In other embodiments, the aforesaid antibody molecules comprise a light chain comprising the amino acid sequence of SEQ ID NO: 182.

In other embodiments, the aforesaid antibody molecules comprise a light chain variable domain comprising the amino acid sequence of SEQ ID NO: 188.

In other embodiments, the aforesaid antibody molecules comprise a light chain comprising the amino acid sequence of SEQ ID NO: 190.

In other embodiments, the aforesaid antibody molecules comprise a light chain variable domain comprising the amino acid sequence of SEQ ID NO: 192.

In other embodiments, the aforesaid antibody molecules comprise a light chain comprising the amino acid sequence of SEQ ID NO: 194.

In other embodiments, the aforesaid antibody molecules comprise a light chain variable domain comprising the amino acid sequence of SEQ ID NO: 196.

In other embodiments, the aforesaid antibodies comprise a light chain comprising the amino acid sequence of SEQ ID NO: 198.

In other embodiments, the aforesaid antibody molecules comprise a light chain variable domain comprising the amino acid sequence of SEQ ID NO: 200.

In other embodiments, the aforesaid antibody molecules comprise a light chain comprising the amino acid sequence of SEQ ID NO: 202.

In other embodiments, the aforesaid antibody molecules comprise a light chain variable domain comprising the amino acid sequence of SEQ ID NO: 204.

In other embodiments, the aforesaid antibody molecules comprise a light chain comprising the amino acid sequence of SEQ ID NO: 206.

In other embodiments, the aforesaid antibody molecules comprise a light chain variable domain comprising the amino acid sequence of SEQ ID NO: 208.

In other embodiments, the aforesaid antibody molecules comprise a light chain comprising the amino acid sequence of SEQ ID NO: 210.

In other embodiments, the aforesaid antibody molecules comprise a light chain variable domain comprising the amino acid sequence of SEQ ID NO: 212.

In other embodiments, the aforesaid antibody molecules comprise a light chain comprising the amino acid sequence of SEQ ID NO: 214.

In other embodiments, the aforesaid antibody molecules comprise a heavy chain variable domain comprising the amino acid sequence of SEQ ID NO: 172 and a light chain variable domain comprising the amino acid sequence of SEQ ID NO: 176.

In other embodiments, the aforesaid antibody molecules comprise a heavy chain variable domain comprising the amino acid sequence of SEQ ID NO: 172 and a light chain variable domain comprising the amino acid sequence of SEQ ID NO: 200.

In other embodiments, the aforesaid antibody molecules comprise a heavy chain variable domain comprising the amino acid sequence of SEQ ID NO: 172 and a light chain variable domain comprising the amino acid sequence of SEQ ID NO: 204.

In other embodiments, the aforesaid antibody molecules comprise a heavy chain variable domain comprising the amino acid sequence of SEQ ID NO: 184 and a light chain variable domain comprising the amino acid sequence of SEQ ID NO: 204.

In other embodiments, the aforesaid antibody molecules comprise a heavy chain variable domain comprising the amino acid sequence of SEQ ID NO: 172 and a light chain variable domain comprising the amino acid sequence of SEQ ID NO: 180.

In other embodiments, the aforesaid antibody molecules comprise a heavy chain variable domain comprising the amino acid sequence of SEQ ID NO: 184 and a light chain variable domain comprising the amino acid sequence of SEQ ID NO: 180.

In other embodiments, the aforesaid antibody molecules comprise a heavy chain variable domain comprising the amino acid sequence of SEQ ID NO: 184 and a light chain variable domain comprising the amino acid sequence of SEQ ID NO: 188.

In other embodiments, the aforesaid antibody molecules comprise a heavy chain variable domain comprising the amino acid sequence of SEQ ID NO: 172 and a light chain variable domain comprising the amino acid sequence of SEQ ID NO: 188.

In other embodiments, the aforesaid antibody molecules comprise a heavy chain variable domain comprising the amino acid sequence of SEQ ID NO: 172 and a light chain variable domain comprising the amino acid sequence of SEQ ID NO: 192.

In other embodiments, the aforesaid antibody molecules comprise a heavy chain variable domain comprising the amino acid sequence of SEQ ID NO: 172 and a light chain variable domain comprising the amino acid sequence of SEQ ID NO: 196.

In other embodiments, the aforesaid antibody molecules comprise a heavy chain variable domain comprising the amino acid sequence of SEQ ID NO: 184 and a light chain variable domain comprising the amino acid sequence of SEQ ID NO: 200.

In other embodiments, the aforesaid antibody molecules comprise a heavy chain variable domain comprising the amino acid sequence of SEQ ID NO: 172 and a light chain variable domain comprising the amino acid sequence of SEQ ID NO: 208.

In other embodiments, the aforesaid antibody molecules comprise a heavy chain variable domain comprising the amino acid sequence of SEQ ID NO: 172 and a light chain variable domain comprising the amino acid sequence of SEQ ID NO: 212.

In other embodiments, the aforesaid antibody molecules comprise a heavy chain variable domain comprising the amino acid sequence of SEQ ID NO: 216 and a light chain variable domain comprising the amino acid sequence of SEQ ID NO: 204.

In other embodiments, the aforesaid antibody molecules comprise a heavy chain variable domain comprising the amino acid sequence of SEQ ID NO: 216 and a light chain variable domain comprising the amino acid sequence of SEQ ID NO: 200.

In other embodiments, the aforesaid antibody molecules comprise a heavy chain variable domain comprising the amino acid sequence of SEQ ID NO: 220 and a light chain variable domain comprising the amino acid sequence of SEQ ID NO: 200.

In other embodiments, the aforesaid antibody molecules comprise a heavy chain comprising the amino acid sequence of SEQ ID NO: 225 and a light chain comprising the amino acid sequence of SEQ ID NO: 178.

In other embodiments, the aforesaid antibody molecules comprise a heavy chain comprising the amino acid sequence of SEQ ID NO: 225 and a light chain comprising the amino acid sequence of SEQ ID NO: 190.

In other embodiments, the aforesaid antibody molecules comprise a heavy chain comprising the amino acid sequence of SEQ ID NO: 225 and a light chain comprising the amino acid sequence of SEQ ID NO: 202.

In other embodiments, the aforesaid antibody molecules comprise a heavy chain comprising the amino acid sequence of SEQ ID NO: 225 and a light chain comprising the amino acid sequence of SEQ ID NO: 206.

In other embodiments, the aforesaid antibody molecules comprise a heavy chain comprising the amino acid sequence of SEQ ID NO: 236 and a light chain comprising the amino acid sequence of SEQ ID NO: 206.

In other embodiments, the aforesaid antibody molecules comprise a heavy chain comprising the amino acid sequence of SEQ ID NO: 174 and a light chain comprising the amino acid sequence of SEQ ID NO: 178.

In other embodiments, the aforesaid antibody molecules comprise a heavy chain comprising the amino acid sequence of SEQ ID NO: 174 and a light chain comprising the amino acid sequence of SEQ ID NO: 182.

In other embodiments, the aforesaid antibody molecules comprise a heavy chain comprising the amino acid sequence of SEQ ID NO: 186 and a light chain comprising the amino acid sequence of SEQ ID NO: 182.

In other embodiments, the aforesaid antibody molecules comprise a heavy chain comprising the amino acid sequence of SEQ ID NO: 186 and a light chain comprising the amino acid sequence of SEQ ID NO: 190.

In other embodiments, the aforesaid antibody molecules comprise a heavy chain comprising the amino acid sequence of SEQ ID NO: 174 and a light chain comprising the amino acid sequence of SEQ ID NO: 190.

In other embodiments, the aforesaid antibodies comprise a heavy chain comprising the amino acid sequence of SEQ ID NO: 174 and a light chain comprising the amino acid sequence of SEQ ID NO: 194.

In other embodiments, the aforesaid antibody molecules comprise a heavy chain comprising the amino acid sequence of SEQ ID NO: 174 and a light chain comprising the amino acid sequence of SEQ ID NO: 198.

In other embodiments, the aforesaid antibody molecules comprise a heavy chain comprising the amino acid sequence of SEQ ID NO: 186 and a light chain comprising the amino acid sequence of SEQ ID NO: 202.

In other embodiments, the aforesaid antibody molecules comprise a heavy chain comprising the amino acid sequence of SEQ ID NO: 174 and a light chain comprising the amino acid sequence of SEQ ID NO: 202.

In other embodiments, the aforesaid antibody molecules comprise a heavy chain comprising the amino acid sequence of SEQ ID NO: 186 and a light chain comprising the amino acid sequence of SEQ ID NO: 206.

In other embodiments, the aforesaid antibody molecules comprise a heavy chain comprising the amino acid sequence of SEQ ID NO: 174 and a light chain comprising the amino acid sequence of SEQ ID NO: 206.

In other embodiments, the aforesaid antibody molecules comprise a heavy chain comprising the amino acid sequence of SEQ ID NO: 174 and a light chain comprising the amino acid sequence of SEQ ID NO: 210.

In other embodiments, the aforesaid antibody molecules comprise a heavy chain comprising the amino acid sequence of SEQ ID NO: 174 and a light chain comprising the amino acid sequence of SEQ ID NO: 214.

In other embodiments, the aforesaid antibody molecules comprise a heavy chain comprising the amino acid sequence of SEQ ID NO: 218 and a light chain comprising the amino acid sequence of SEQ ID NO: 206.

In other embodiments, the aforesaid antibodies comprise a heavy chain comprising the amino acid sequence of SEQ ID NO: 218 and a light chain comprising the amino acid sequence of SEQ ID NO: 202.

In other embodiments, the aforesaid antibody molecules comprise a heavy chain comprising the amino acid sequence of SEQ ID NO: 222 and a light chain comprising the amino acid sequence of SEQ ID NO: 202.

In other embodiments, the aforesaid antibody molecules are chosen from a Fab, F(ab′)2, Fv, or a single chain Fv fragment (scFv).

In other embodiments, the aforesaid antibody molecules comprise a heavy chain constant region selected from IgG1, IgG2, IgG3, and IgG4.

In other embodiments, the aforesaid antibody molecules comprise a light chain constant region chosen from the light chain constant regions of kappa or lambda.

In other embodiments, the aforesaid antibody molecules comprise a human IgG4 heavy chain constant region with a mutation at position 228 according to EU numbering or position 108 of SEQ ID NO: 212 or 214 of US 2015/0210769A1 and a kappa light chain constant region.

In other embodiments, the aforesaid antibody molecules comprise a human IgG4 heavy chain constant region with a Serine to Proline mutation at position 228 according to EU numbering or position 108 of SEQ ID NO: 212 or 214 of US 2015/0210769A1 and a kappa light chain constant region.

In other embodiments, the aforesaid antibody molecules comprise a human IgG1 heavy chain constant region with an Asparagine to Alanine mutation at position 297 according to EU numbering or position 180 of SEQ ID NO: 216 of US 2015/0210769A1 and a kappa light chain constant region.

In other embodiments, the aforesaid antibody molecules comprise a human IgG1 heavy chain constant region with an Aspartate to Alanine mutation at position 265 according to EU numbering or position 148 of SEQ ID NO: 217 of US 2015/0210769A1, and Proline to Alanine mutation at position 329 according to EU numbering or position 212 of SEQ ID NO: 217 of US 2015/0210769A1 and a kappa light chain constant region.

In other embodiments, the aforesaid antibody molecules comprise a human IgG1 heavy chain constant region with a Leucine to Alanine mutation at position 234 according to EU numbering or position 117 of SEQ ID NO: 218 of US 2015/0210769A1, and Leucine to Alanine mutation at position 235 according to EU numbering or position 118 of SEQ ID NO: 218 of US 2015/0210769A1 and a kappa light chain constant region.

In other embodiments, the aforesaid antibody molecules are capable of binding to human PD-1 with a dissociation constant (K_{D}) of less than about 0.2 nM.

In some embodiments, the aforesaid antibody molecules bind to human PD-1 with a K_{D} of less than about 0.2 nM, 0.15 nM, 0.1 nM, 0.05 nM, or 0.02 nM, e.g., about 0.13 nM to 0.03 nM, e.g., about 0.077 nM to 0.088 nM, e.g., about 0.083 nM, e.g., as measured by a Biacore method.

In other embodiments, the aforesaid antibody molecules bind to cynomolgus PD-1 with a K_{D} of less than about 0.2 nM, 0.15 nM, 0.1 nM, 0.05 nM, or 0.02 nM, e.g., about 0.11 nM to 0.08 nM, e.g., about 0.093 nM, e.g., as measured by a Biacore method.

In certain embodiments, the aforesaid antibody molecules bind to both human PD-1 and cynomolgus PD-1 with similar K_{D}, e.g., in the nM range, e.g., as measured by a Biacore method. In some embodiments, the aforesaid antibody molecules bind to a human PD-1-Ig fusion protein with a K_{D} of less than about 0.1 nM, 0.075 nM, 0.05 nM, 0.025 nM, or 0.01 nM, e.g., about 0.04 nM, e.g., as measured by ELISA.

In some embodiments, the aforesaid antibody molecules bind to Jurkat cells that express human PD-1 (e.g., human PD-1-transfected Jurkat cells) with a K_{D} of less than about 0.1 nM, 0.075 nM, 0.05 nM, 0.025 nM, or 0.01 nM, e.g., about 0.06 nM, e.g., as measured by FACS analysis.

In some embodiments, the aforesaid antibody molecules bind to cynomolgus T cells with a K_{D} of less than about 1 nM, 0.75 nM, 0.5 nM, 0.25 nM, or 0.1 nM, e.g., about 0.4 nM, e.g., as measured by FACS analysis.

In some embodiments, the aforesaid antibody molecules bind to cells that express cynomolgus PD-1 (e.g., cells transfected with cynomolgus PD-1) with a K_{D} of less than about 1 nM, 0.75 nM, 0.5 nM, 0.25 nM, or 0.01 nM, e.g., about 0.6 nM, e.g., as measured by FACS analysis.

In certain embodiments, the aforesaid antibody molecules are not cross-reactive with mouse or rat PD-1. In other embodiments, the aforesaid antibodies are cross-reactive with rhesus PD-1. For example, the cross-reactivity can be measured by a Biacore method or a binding assay using cells that expresses PD-1 (e.g., human PD-1-expressing 300.19 cells). In other embodiments, the aforesaid antibody molecules bind an extracellular Ig-like domain of PD-1.

In other embodiments, the aforesaid antibody molecules are capable of reducing binding of PD-1 to PD-L1, PD-L2, or both, or a cell that expresses PD-L1, PD-L2, or both. In some embodiments, the aforesaid antibody molecules reduce (e.g., block) PD-L1 binding to a cell that expresses PD-1 (e.g., human PD-1-expressing 300.19 cells) with an IC50 of less than about 1.5 nM, 1 nM, 0.8 nM, 0.6 nM, 0.4 nM, 0.2 nM, or 0.1 nM, e.g., between about 0.79 nM and about 1.09 nM, e.g., about 0.94 nM, or about 0.78 nM or less, e.g., about 0.3 nM. In some embodiments, the aforesaid antibodies reduce (e.g., block) PD-L2 binding to a cell that expresses PD-1 (e.g., human PD-1-expressing 300.19 cells) with an IC50 of less than about 2 nM, 1.5 nM, 1 nM, 0.5 nM, or 0.2 nM, e.g., between about 1.05 nM and about 1.55 nM, or about 1.3 nM or less, e.g., about 0.9 nM.

In other embodiments, the aforesaid antibody molecules are capable of enhancing an antigen-specific T cell response.

In embodiments, the antibody molecule is a monospecific antibody molecule or a bispecific antibody molecule. In embodiments, the antibody molecule has a first binding specificity for PD-1 and a second binding specifity for TIM-3, LAG-3, CEACAM (e.g., CEACAM-1, CEACAM-3, and/or CEACAM-5), PD-L1 or PD-L2. In embodiments, the antibody molecule comprises an antigen binding fragment of an antibody, e.g., a half antibody or antigen binding fragment of a half antibody.

In some embodiments, the aforesaid antibody molecules increase the expression of IL-2 from cells activated by Staphylococcal enterotoxin B (SEB) (e.g., at 25 μg/mL) by at least about 2, 3, 4, 5-fold, e.g., about 2 to 3-fold, e.g., about 2 to 2.6-fold, e.g., about 2.3-fold, compared to the expression of IL-2 when an isotype control (e.g., IgG4) is used, e.g., as measured in a SEB T cell activation assay or a human whole blood ex vivo assay.

In some embodiments, the aforesaid antibody molecules increase the expression of IFN-γ from T cells stimulated by anti-CD3 (e.g., at 0.1 μg/mL) by at least about 2, 3, 4, 5-fold, e.g., about 1.2 to 3.4-fold, e.g., about 2.3-fold, compared to the expression of IFN-γ when an isotype control (e.g., IgG4) is used, e.g., as measured in an IFN-γ activity assay.

In some embodiments, the aforesaid antibody molecules increase the expression of IFN-γ from T cells activated by SEB (e.g., at 3 pg/mL) by at least about 2, 3, 4, 5-fold, e.g., about 0.5 to 4.5-fold, e.g., about 2.5-fold, compared to the expression of IFN-γ when an isotype control (e.g., IgG4) is used, e.g., as measured in an IFN-γ activity assay.

In some embodiments, the aforesaid antibody molecules increase the expression of IFN-γ from T cells activated with an CMV peptide by at least about 2, 3, 4, 5-fold, e.g., about 2 to 3.6-fold, e.g., about 2.8-fold, compared to the expression of IFN-γ when an isotype control (e.g., IgG4) is used, e.g., as measured in an IFN-γ activity assay.

In some embodiments, the aforesaid antibody molecules increase the proliferation of CD8⁺ T cells activated with an CMV peptide by at least about 1, 2, 3, 4, 5-fold, e.g., about 1.5-fold, compared to the proliferation of CD8⁺ T cells when an isotype control (e.g., IgG4) is used, e.g., as measured by the percentage of CD8+ T cells that passed through at least n (e.g., n=2 or 4) cell divisions.

In certain embodiments, the aforesaid antibody molecules has a Cmax between about 100 μg/mL and about 500 μg/mL, between about 150 μg/mL and about 450 μg/mL, between about 250 μg/mL and about 350 μg/mL, or between about 200 μg/mL and about 400 μg/mL, e.g., about 292.5 μg/mL, e.g., as measured in monkey.

In certain embodiments, the aforesaid antibody molecules has a T_{1/2} between about 250 hours and about 650 hours, between about 300 hours and about 600 hours, between about 350 hours and about 550 hours, or between about 400 hours and about 500 hours, e.g., about 465.5 hours, e.g., as measured in monkey.

In some embodiments, the aforesaid antibody molecules bind to PD-1 with a Kd slower than 5×10⁻⁴, 1×10⁻⁴, 5×10⁻⁵, or 1×10⁻⁵ s⁻¹, e.g., about 2.13×10⁻⁴ s⁻¹, e.g., as measured by a Biacore method. In some embodiments, the aforesaid antibody molecules bind to PD-1 with a Ka faster than 1×10⁴, 5×10⁴, 1×10⁵, or 5×10⁵ M⁻¹s⁻¹, e.g., about 2.78×10⁵ M⁻¹s⁻¹, e.g., as measured by a Biacore method.

In some embodiments, the aforesaid anti-PD-1 antibody molecules bind to one or more residues within the C strand, CC′ loop, C′ strand and FG loop of PD-1. The domain structure of PD-1 is described, e.g., in Cheng et al., “Structure and Interactions of the Human Programmed Cell Death 1 Receptor” J. Biol. Chem. 2013, 288:11771-11785. As described in Cheng et. al., the C strand comprises residues F43-M50, the CC′ loop comprises S51-N54, the C′ strand comprises residues Q55-F62, and the FG loop comprises residues L108-I114 (amino acid numbering according to Chang et al. supra). Accordingly, in some embodiments, an anti-PD-1 antibody as described herein binds to at least one residue in one or more of the ranges F43-M50, S51-N54, Q55-F62, and L1084114 of PD-1. In some embodiments, an anti-PD-1 antibody as described herein binds to at least one residue in two, three, or all four of the ranges F43-M50, S51-N54, Q55-F62, and L1084114 of PD-1. In some embodiments, the anti-PD-1 antibody binds to a residue in PD-1 that is also part of a binding site for one or both of PD-L1 and PD-L2.

In another aspect, the invention provides an isolated nucleic acid molecule encoding any of the aforesaid antibody molecules, vectors and host cells thereof.

An isolated nucleic acid encoding the antibody heavy chain variable region or light chain variable region, or both, of any the aforesaid antibody molecules is also provided.

In one embodiment, the isolated nucleic acid encodes heavy chain CDRs 1-3, wherein said nucleic acid comprises a nucleotide sequence of SEQ ID NO: 242-246, 255, 256-260, 267-271, or 278-280.

In another embodiment, the isolated nucleic acid encodes light chain CDRs 1-3, wherein said nucleic acid comprises a nucleotide sequence of SEQ ID NO: 247-254, 261-266, or 272-277.

In other embodiments, the aforesaid nucleic acid further comprises a nucleotide sequence encoding a heavy chain variable domain, wherein said nucleotide sequence is at least 85% identical to any of SEQ ID NO: 173, 185, 217, 221, 224, 229, or 235.

In other embodiments, the aforesaid nucleic acid further comprises a nucleotide sequence encoding a heavy chain variable domain, wherein said nucleotide sequence comprises any of SEQ ID NO: 173, 185, 217, 221, 224, 229, or 235.

In other embodiments, the aforesaid nucleic acid further comprises a nucleotide sequence encoding a heavy chain, wherein said nucleotide sequence is at least 85% identical to any of SEQ ID NO: 175, 187, 219, 223, 226, 230, or 237.

In other embodiments, the aforesaid nucleic acid further comprises a nucleotide sequence encoding a heavy chain, wherein said nucleotide sequence comprises any of SEQ ID NO: 175, 187, 219, 223, 226, 230, or 237.

In other embodiments, the aforesaid nucleic acid further comprises a nucleotide sequence encoding a light chain variable domain, wherein said nucleotide sequence is at least 85% identical to any of SEQ ID NO: 177, 181, 189, 193, 197, 201, 205, 209, 213, 227, 231, 233, 238, or 240.

In other embodiments, the aforesaid nucleic acid further comprises a nucleotide sequence encoding a light chain variable domain, wherein said nucleotide sequence comprises any of SEQ ID NO: 177, 181, 189, 193, 197, 201, 205, 209, 213, 227, 231, 233, 238, or 240.

In other embodiments, the aforesaid nucleic acid further comprises a nucleotide sequence encoding a light chain, wherein said nucleotide sequence is at least 85% identical to any of SEQ ID NO: 179, 183, 191, 195, 199, 203, 207, 211, 215, 228, 232, 234, 239 or 241.

In other embodiments, the aforesaid nucleic acid further comprises a nucleotide sequence encoding a light chain, wherein said nucleotide sequence comprises any of SEQ ID NO: 179, 183, 191, 195, 199, 203, 207, 211, 215, 228, 232, 234, 239 or 241.

In certain embodiments, one or more expression vectors and host cells comprising the aforesaid nucleic acids are provided.

A method of producing an antibody molecule or fragment thereof, comprising culturing the host cell as described herein under conditions suitable for gene expression is also provided.

In one aspect, the disclosure features a method of providing an antibody molecule described herein. The method includes: providing a PD-1 antigen (e.g., an antigen comprising at least a portion of a PD-1 epitope); obtaining an antibody molecule that specifically binds to the PD-1 polypeptide; and evaluating if the antibody molecule specifically binds to the PD-1 polypeptide, or evaluating efficacy of the antibody molecule in modulating, e.g., inhibiting, the activity of the PD-1. The method can further include administering the antibody molecule to a subject, e.g., a human or non-human animal.

In another aspect, the disclosure provides compositions, e.g., pharmaceutical compositions, which include a pharmaceutically acceptable carrier, excipient or stabilizer, and at least one of the therapeutic agents, e.g., anti-PD-1 antibody molecules described herein. In one embodiment, the composition, e.g., the pharmaceutical composition, includes a combination of the antibody molecule and one or more agents, e.g., a therapeutic agent or other antibody molecule, as described herein. In one embodiment, the antibody molecule is conjugated to a label or a therapeutic agent.

TABLE 6
Amino acid and nucleotide sequences for murine, chimeric and humanized anti-PD-1
antibody molecules. The antibody molecules include murine mAb BAP049, chimeric mAbs
BAP049-chi and BAP049-chi-Y, and humanized mAbs BAP049-hum01 to BAP049-hum16 and
BAP049-Clone-A to BAP049-Clone-E. The amino acid and nucleotide sequences of the heavy
and light chain CDRs, the heavy and light chain variable regions, and the heavy and light
chains are shown.

| BAP049 HC | | |
|---|---|---|
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 | HCDR1 | GYTFTTY |
| (Chothia) | | |
| SEQ ID NO: 141 | HCDR2 | YPGTGG |
| (Chothia) | | |
| SEQ ID NO: 139 | HCDR3 | WTTGTGAY |
| (Chothia) | | |
| SEQ ID NO: 142 | VH | QVQLQQPGSELVRPGASVKLS |
| | | CKASGYTFTTYWMHWVRQRPGQGL |
| | | EWIGNIYPGTGGSNFDEKFKNRTSLT |
| | | VDTSSTTAYMHLASLTSEDSAVYYC |
| | | TRWTTGTGAYWGQGTLVTVSA |
| SEQ ID NO: 143 | DNA VH | CAGGTCCAGCTGCAGCAACC |
| | | TGGGTCTGAGCTGGTGAGGCCTGG |
| | | AGCTTCAGTGAAGCTGTCCTGCAA |
| | | GGCGTCTGGCTACACATTCACCACT |
| | | TACTGGATGCACTGGGTGAGGCAG |
| | | AGGCCTGGACAAGGCCTTGAGTGG |
| | | ATTGGAAATATTTATCCTGGTACTG |
| | | GTGGTTCTAACTTCGATGAGAAGTT |
| | | CAAAAACAGGACCTCACTGACTGT |
| | | AGACACATCCTCCACCACAGCCTA |
| | | CATGCACCTCGCCAGCCTGACATCT |
| | | GAGGACTCTGCGGTCTATTACTGTA |
| | | CAAGATGGACTACTGGGACGGGAG |
| | | CTTATTGGGGCCAAGGGACTCTGG |
| | | TCACTGTCTCTGCA |
| SEQ ID NO: 144 | VH | QVQLQQSGSELVRPGASVKLS |
| | | CKASGYTFTTYWMHWVRQRPGQGL |
| | | EWIGNIYPGTGGSNFDEKFKNRTSLT |
| | | VDTSSTTAYMHLASLTSEDSAVYYC |
| | | TRWTTGTGAYWGQGTLVTVSA |
| SEQ ID NO: 145 | DNA VH | CAGGTCCAGCTGCAGCAGTC |
| | | TGGGTCTGAGCTGGTGAGGCCTGG |
| | | AGCTTCAGTGAAGCTGTCCTGCAA |
| | | GGCGTCTGGCTACACATTCACCACT |
| | | TACTGGATGCACTGGGTGAGGCAG |
| | | AGGCCTGGACAAGGCCTTGAGTGG |
| | | ATTGGAAATATTTATCCTGGTACTG |
| | | GTGGTTCTAACTTCGATGAGAAGTT |
| | | CAAAAACAGGACCTCACTGACTGT |
| | | AGACACATCCTCCACCACAGCCTA |
| | | CATGCACCTCGCCAGCCTGACATCT |
| | | GAGGACTCTGCGGTCTATTACTGTA |
| | | CAAGATGGACTACTGGGACGGGAG |
| | | CTTATTGGGGCCAAGGGACTCTGG |
| | | TCACTGTCTCTGCA |
| BAP049 LC | | |
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 148 (Kabat) | LCDR3 | QNDYSYPCT |
| SEQ ID NO: 149 | LCDR1 | SQSLLDSGNQKNF |
| (Chothia) | | |
| SEQ ID NO: 150 | LCDR2 | WAS |
| (Chothia) | | |
| SEQ ID NO: 151 | LCDR3 | DYSYPC |
| (Chothia) | | |
| SEQ ID NO: 152 | VL | DIVMTQSPSSLTVTAGEKVTM |
| | | SCKSSQSLLDSGNQKNFLTWYQQKP |
| | | GQPPKLLIFWASTRESGVPDRFTGSG |
| | | SVTDFTLTISSVQAEDLAVYYCQND |
| | | YSYPCTFGGGTKLEIK |
| SEQ ID NO: 153 | DNA VL | GACATTGTGATGACCCAGTC |
| | | TCCATCCTCCCTGACTGTGACAGCA |
| | | GGAGAGAAGGTCACTATGAGCTGC |
| | | AAGTCCAGTCAGAGTCTGTTAGAC |
| | | AGTGGAAATCAAAAGAACTTCTTG |
| | | ACCTGGTACCAGCAGAAACCAGGG |
| | | CAGCCTCCTAAACTGTTGATCTTCT |
| | | GGGCATCCACTAGGGAATCTGGGG |
| | | TCCCTGATCGCTTCACAGGCAGTGG |
| | | ATCTGTAACAGATTTCACTCTCACC |
| | | ATCAGCAGTGTGCAGGCTGAAGAC |
| | | CTGGCAGTTTATTACTGTCAGAATG |
| | | ATTATAGTTATCCGTGCACGTTCGG |
| | | AGGGGGGACCAAGCTGGAAATAAA |
| | | A |
| BAP049-chi HC | | |
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 | HCDR1 | GYTFTTY |
| (Chothia) | | |
| SEQ ID NO: 141 | HCDR2 | YPGTGG |
| (Chothia) | | |
| SEQ ID NO: 139 | HCDR3 | WTTGTGAY |
| (Chothia) | | |
| SEQ ID NO: 154 | VH | QVQLQQPGSELVRPGASVKLS |
| | | CKASGYTFTTYWMHWVRQRPGQGL |
| | | EWIGNIYPGTGGSNFDEKFKNRTSLT |
| | | VDTSSTTAYMHLASLTSEDSAVYYC |
| | | TRWTTGTGAYWGQGTTVTVSS |
| SEQ ID NO: 155 | DNA VH | CAGGTCCAGCTGCAGCAGCC |
| | | TGGGTCTGAGCTGGTGAGGCCTGG |
| | | AGCTTCAGTGAAGCTGTCCTGCAA |
| | | GGCGTCTGGCTACACATTCACCACT |
| | | TACTGGATGCACTGGGTGAGGCAG |
| | | AGGCCTGGACAAGGCCTTGAGTGG |
| | | ATTGGAAATATTTATCCTGGTACTG |
| | | GTGGTTCTAACTTCGATGAGAAGTT |
| | | CAAAAACAGGACCTCACTGACTGT |
| | | AGACACATCCTCCACCACAGCCTA |
| | | CATGCACCTCGCCAGCCTGACATCT |
| | | GAGGACTCTGCGGTCTATTACTGTA |
| | | CAAGATGGACTACTGGGACGGGAG |
| | | CTTATTGGGGCCAGGGCACCACCG |
| | | TGACCGTGTCCTCC |
| SEQ ID NO: 156 | HC | QVQLQQPGSELVRPGASVKLS |
| | | CKASGYTFTTYWMHWVRQRPGQGL |
| | | EWIGNIYPGTGGSNFDEKFKNRTSLT |
| | | VDTSSTTAYMHLASLTSEDSAVYYC |
| | | TRWTTGTGAYWGQGTTVTVSSAST |
| | | KGPSVFPLAPCSRSTSESTAALGCLV |
| | | KDYFPEPVTVSWNSGALTSGVHTFP |
| | | AVLQSSGLYSLSSVVTVPSSSLGTKT |
| | | YTCNVDHKPSNTKVDKRVESKYGPP |
| | | CPPCPAPEFLGGPSVFLFPPKPKDTL |
| | | MISRTPEVTCVVVDVSQEDPEVQFN |
| | | WYVDGVEVHNAKTKPREEQFNSTY |
| | | RVVSVLTVLHQDWLNGKEYKCKVS |
| | | NKGLPSSIEKTISKAKGQPREPQVYT |
| | | LPPSQEEMTKNQVSLTCLVKGFYPSD |
| | | IAVEWESNGQPENNYKTTPPVLDSD |
| | | GSFFLYSRLTVDKSRWQEGNVFSCS |
| | | VMHEALHNHYTQKSLSLSLGK |
| SEQ ID NO: 157 | DNA HC | CAGGTCCAGCTGCAGCAGCC |
| | | TGGGTCTGAGCTGGTGAGGCCTGG |
| | | AGCTTCAGTGAAGCTGTCCTGCAA |
| | | GGCGTCTGGCTACACATTCACCACT |
| | | TACTGGATGCACTGGGTGAGGCAG |
| | | AGGCCTGGACAAGGCCTTGAGTGG |
| | | ATTGGAAATATTTATCCTGGTACTG |
| | | GTGGTTCTAACTTCGATGAGAAGTT |
| | | CAAAAACAGGACCTCACTGACTGT |
| | | AGACACATCCTCCACCACAGCCTA |
| | | CATGCACCTCGCCAGCCTGACATCT |
| | | GAGGACTCTGCGGTCTATTACTGTA |
| | | CAAGATGGACTACTGGGACGGGAG |
| | | CTTATTGGGGCCAGGGCACCACCG |
| | | TGACCGTGTCCTCCGCTTCCACCAA |
| | | GGGCCCATCCGTCTTCCCCCTGGCG |
| | | CCCTGCTCCAGGAGCACCTCCGAG |
| | | AGCACAGCCGCCCTGGGCTGCCTG |
| | | GTCAAGGACTACTTCCCCGAACCG |
| | | GTGACGGTGTCGTGGAACTCAGGC |
| | | GCCCTGACCAGCGGCGTGCACACC |
| | | TTCCCGGCTGTCCTACAGTCCTCAG |
| | | GACTCTACTCCCTCAGCAGCGTGGT |
| | | GACCGTGCCCTCCAGCAGCTTGGG |
| | | CACGAAGACCTACACCTGCAACGT |
| | | AGATCACAAGCCCAGCAACACCAA |
| | | GGTGGACAAGAGAGTTGAGTCCAA |
| | | ATATGGTCCCCCATGCCCACCGTGC |
| | | CCAGCACCTGAGTTCCTGGGGGGA |
| | | CCATCAGTCTTCCTGTTCCCCCCAA |
| | | AACCCAAGGACACTCTCATGATCT |
| | | CCCGGACCCCTGAGGTCACGTGCG |
| | | TGGTGGTGGACGTGAGCCAGGAAG |
| | | ACCCCGAGGTCCAGTTCAACTGGT |
| | | ACGTGGATGGCGTGGAGGTGCATA |
| | | ATGCCAAGACAAAGCCGCGGGAGG |
| | | AGCAGTTCAACAGCACGTACCGTG |
| | | TGGTCAGCGTCCTCACCGTCCTGCA |
| | | CCAGGACTGGCTGAACGGCAAGGA |
| | | GTACAAGTGCAAGGTGTCCAACAA |
| | | AGGCCTCCCGTCCTCCATCGAGAA |
| | | AACCATCTCCAAAGCCAAAGGGCA |
| | | GCCCCGAGAGCCACAGGTGTACAC |
| | | CCTGCCCCCATCCCAGGAGGAGAT |
| | | GACCAAGAACCAGGTCAGCCTGAC |
| | | CTGCCTGGTCAAAGGCTTCTACCCC |
| | | AGCGACATCGCCGTGGAGTGGGAG |
| | | AGCAATGGGCAGCCGGAGAACAAC |
| | | TACAAGACCACGCCTCCCGTGCTG |
| | | GACTCCGACGGCTCCTTCTTCCTCT |
| | | ACAGCAGGCTAACCGTGGACAAGA |
| | | GCAGGTGGCAGGAGGGGAATGTCT |
| | | TCTCATGCTCCGTGATGCATGAGGC |
| | | TCTGCACAACCACTACACACAGAA |
| | | GAGCCTCTCCCTGTCTCTGGGTAAA |
| SEQ ID NO: 158 | VH | QVQLQQSGSELVRPGASVKLS |
| | | CKASGYTFTTYWMHWVRQRPGQGL |
| | | EWIGNIYPGTGGSNFDEKFKNRTSLT |
| | | VDTSSTTAYMHLASLTSEDSAVYYC |
| | | TRWTTGTGAYWGQGTTVTVSS |
| SEQ ID NO: 159 | DNA VH | CAGGTCCAGCTGCAGCAGTC |
| | | TGGGTCTGAGCTGGTGAGGCCTGG |
| | | AGCTTCAGTGAAGCTGTCCTGCAA |
| | | GGCGTCTGGCTACACATTCACCACT |
| | | TACTGGATGCACTGGGTGAGGCAG |
| | | AGGCCTGGACAAGGCCTTGAGTGG |
| | | ATTGGAAATATTTATCCTGGTACTG |
| | | GTGGTTCTAACTTCGATGAGAAGTT |
| | | CAAAAACAGGACCTCACTGACTGT |
| | | AGACACATCCTCCACCACAGCCTA |
| | | CATGCACCTCGCCAGCCTGACATCT |
| | | GAGGACTCTGCGGTCTATTACTGTA |
| | | CAAGATGGACTACTGGGACGGGAG |
| | | CTTATTGGGGCCAGGGCACCACCG |
| | | TGACCGTGTCCTCC |
| SEQ ID NO: 160 | HC | QVQLQQSGSELVRPGASVKLS |
| | | CKASGYTFTTYWMHWVRQRPGQGL |
| | | EWIGNIYPGTGGSNFDEKFKNRTSLT |
| | | VDTSSTTAYMHLASLTSEDSAVYYC |
| | | TRWTTGTGAYWGQGTTVTVSSAST |
| | | KGPSVFPLAPCSRSTSESTAALGCLV |
| | | KDYFPEPVTVSWNSGALTSGVHTFP |
| | | AVLQSSGLYSLSSVVTVPSSSLGTKT |
| | | YTCNVDHKPSNTKVDKRVESKYGPP |
| | | CPPCPAPEFLGGPSVFLFPPKPKDTL |
| | | MISRTPEVTCVVVDVSQEDPEVQFN |
| | | WYVDGVEVHNAKTKPREEQFNSTY |
| | | RVVSVLTVLHQDWLNGKEYKCKVS |
| | | NKGLPSSIEKTISKAKGQPREPQVYT |
| | | LPPSQEEMTKNQVSLTCLVKGFYPSD |
| | | IAVEWESNGQPENNYKTTPPVLDSD |
| | | GSFFLYSRLTVDKSRWQEGNVFSCS |
| | | VMHEALHNHYTQKSLSLSLGK |
| SEQ ID NO: 161 | DNA HC | CAGGTCCAGCTGCAGCAGTC |
| | | TGGGTCTGAGCTGGTGAGGCCTGG |
| | | AGCTTCAGTGAAGCTGTCCTGCAA |
| | | GGCGTCTGGCTACACATTCACCACT |
| | | TACTGGATGCACTGGGTGAGGCAG |
| | | AGGCCTGGACAAGGCCTTGAGTGG |
| | | ATTGGAAATATTTATCCTGGTACTG |
| | | GTGGTTCTAACTTCGATGAGAAGTT |
| | | CAAAAACAGGACCTCACTGACTGT |
| | | AGACACATCCTCCACCACAGCCTA |
| | | CATGCACCTCGCCAGCCTGACATCT |
| | | GAGGACTCTGCGGTCTATTACTGTA |
| | | CAAGATGGACTACTGGGACGGGAG |
| | | CTTATTGGGGCCAGGGCACCACCG |
| | | TGACCGTGTCCTCCGCTTCCACCAA |
| | | GGGCCCATCCGTCTTCCCCCTGGCG |
| | | CCCTGCTCCAGGAGCACCTCCGAG |
| | | AGCACAGCCGCCCTGGGCTGCCTG |
| | | GTCAAGGACTACTTCCCCGAACCG |
| | | GTGACGGTGTCGTGGAACTCAGGC |
| | | GCCCTGACCAGCGGCGTGCACACC |
| | | TTCCCGGCTGTCCTACAGTCCTCAG |
| | | GACTCTACTCCCTCAGCAGCGTGGT |
| | | GACCGTGCCCTCCAGCAGCTTGGG |
| | | CACGAAGACCTACACCTGCAACGT |
| | | AGATCACAAGCCCAGCAACACCAA |
| | | GGTGGACAAGAGAGTTGAGTCCAA |
| | | ATATGGTCCCCCATGCCCACCGTGC |
| | | CCAGCACCTGAGTTCCTGGGGGGA |
| | | CCATCAGTCTTCCTGTTCCCCCCAA |
| | | AACCCAAGGACACTCTCATGATCT |
| | | CCCGGACCCCTGAGGTCACGTGCG |
| | | TGGTGGTGGACGTGAGCCAGGAAG |
| | | ACCCCGAGGTCCAGTTCAACTGGT |
| | | ACGTGGATGGCGTGGAGGTGCATA |
| | | ATGCCAAGACAAAGCCGCGGGAGG |
| | | AGCAGTTCAACAGCACGTACCGTG |
| | | TGGTCAGCGTCCTCACCGTCCTGCA |
| | | CCAGGACTGGCTGAACGGCAAGGA |
| | | GTACAAGTGCAAGGTGTCCAACAA |
| | | AGGCCTCCCGTCCTCCATCGAGAA |
| | | AACCATCTCCAAAGCCAAAGGGCA |
| | | GCCCCGAGAGCCACAGGTGTACAC |
| | | CCTGCCCCCATCCCAGGAGGAGAT |
| | | GACCAAGAACCAGGTCAGCCTGAC |
| | | CTGCCTGGTCAAAGGCTTCTACCCC |
| | | AGCGACATCGCCGTGGAGTGGGAG |
| | | AGCAATGGGCAGCCGGAGAACAAC |
| | | TACAAGACCACGCCTCCCGTGCTG |
| | | GACTCCGACGGCTCCTTCTTCCTCT |
| | | ACAGCAGGCTAACCGTGGACAAGA |
| | | GCAGGTGGCAGGAGGGGAATGTCT |
| | | TCTCATGCTCCGTGATGCATGAGGC |
| | | TCTGCACAACCACTACACACAGAA |
| | | GAGCCTCTCCCTGTCTCTGGGTAAA |
| BAP049-chi LC | | |
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 148 (Kabat) | LCDR3 | QNDYSYPCT |
| SEQ ID NO: 49 | LCDR1 | SQSLLDSGNQKNF |
| (Chothia) | | |
| SEQ ID NO: 150 | LCDR2 | WAS |
| (Chothia) | | |
| SEQ ID NO: 151 | LCDR3 | DYSYPC |
| (Chothia) | | |
| SEQ ID NO: 162 | VL | DIVMTQSPSSLTVTAGEKVTM |
| | | SCKSSQSLLDSGNQKNFLTWYQQKP |
| | | GQPPKLLIFWASTRESGVPDRFTGSG |
| | | SVTDFTLTISSVQAEDLAVYYCQND |
| | | YSYPCTFGQGTKVEIK |
| SEQ ID NO: 163 | DNA VL | GACATTGTGATGACCCAGTC |
| | | TCCATCCTCCCTGACTGTGACAGCA |
| | | GGAGAGAAGGTCACTATGAGCTGC |
| | | AAGTCCAGTCAGAGTCTGTTAGAC |
| | | AGTGGAAATCAAAAGAACTTCTTG |
| | | ACCTGGTACCAGCAGAAACCAGGG |
| | | CAGCCTCCTAAACTGTTGATCTTCT |
| | | GGGCATCCACTAGGGAATCTGGGG |
| | | TCCCTGATCGCTTCACAGGCAGTGG |
| | | ATCTGTAACAGATTTCACTCTCACC |
| | | ATCAGCAGTGTGCAGGCTGAAGAC |
| | | CTGGCAGTTTATTACTGTCAGAATG |
| | | ATTATAGTTATCCGTGCACGTTCGG |
| | | CCAAGGGACCAAGGTGGAAATCAA |
| | | A |
| SEQ ID NO: 164 | LC | DIVMTQSPSSLTVTAGEKVTM |
| | | SCKSSQSLLDSGNQKNFLTWYQQKP |
| | | GQPPKLLIFWASTRESGVPDRFTGSG |
| | | SVTDFTLTISSVQAEDLAVYYCQND |
| | | YSYPCTFGQGTKVEIKRTVAAPSVFI |
| | | FPPSDEQLKSGTASVVCLLNNFYPRE |
| | | AKVQWKVDNALQSGNSQESVTEQD |
| | | SKDSTYSLSSTLTLSKADYEKHKVY |
| | | ACEVTHQGLSSPVTKSFNRGEC |
| SEQ ID NO: 165 | DNA LC | GACATTGTGATGACCCAGTC |
| | | TCCATCCTCCCTGACTGTGACAGCA |
| | | GGAGAGAAGGTCACTATGAGCTGC |
| | | AAGTCCAGTCAGAGTCTGTTAGAC |
| | | AGTGGAAATCAAAAGAACTTCTTG |
| | | ACCTGGTACCAGCAGAAACCAGGG |
| | | CAGCCTCCTAAACTGTTGATCTTCT |
| | | GGGCATCCACTAGGGAATCTGGGG |
| | | TCCCTGATCGCTTCACAGGCAGTGG |
| | | ATCTGTAACAGATTTCACTCTCACC |
| | | ATCAGCAGTGTGCAGGCTGAAGAC |
| | | CTGGCAGTTTATTACTGTCAGAATG |
| | | ATTATAGTTATCCGTGCACGTTCGG |
| | | CCAAGGGACCAAGGTGGAAATCAA |
| | | ACGTACGGTGGCTGCACCATCTGTC |
| | | TTCATCTTCCCGCCATCTGATGAGC |
| | | AGTTGAAATCTGGAACTGCCTCTGT |
| | | TGTGTGCCTGCTGAATAACTTCTAT |
| | | CCCAGAGAGGCCAAAGTACAGTGG |
| | | AAGGTGGATAACGCCCTCCAATCG |
| | | GGTAACTCCCAGGAGAGTGTCACA |
| | | GAGCAGGACAGCAAGGACAGCACC |
| | | TACAGCCTCAGCAGCACCCTGACG |
| | | CTGAGCAAAGCAGACTACGAGAAA |
| | | CACAAAGTCTACGCCTGCGAAGTC |
| | | ACCCATCAGGGCCTGAGCTCGCCC |
| | | GTCACAAAGAGCTTCAACAGGGGA |
| | | GAGTGT |
| BAP049-chi-Y HC | | |
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 | HCDR1 | GYTFTTY |
| (Chothia) | | |
| SEQ ID NO: 141 | HCDR2 | YPGTGG |
| (Chothia) | | |
| SEQ ID NO: 139 | HCDR3 | WTTGTGAY |
| (Chothia) | | |
| SEQ ID NO: 154 | VH | QVQLQQPGSELVRPGASVKLS |
| | | CKASGYTFTTYWMHWVRQRPGQGL |
| | | EWIGNIYPGTGGSNFDEKFKNRTSLT |
| | | VDTSSTTAYMHLASLTSEDSAVYYC |
| | | TRWTTGTGAYWGQGTTVTVSS |
| SEQ ID NO: 155 | DNA VH | CAGGTCCAGCTGCAGCAGCC |
| | | TGGGTCTGAGCTGGTGAGGCCTGG |
| | | AGCTTCAGTGAAGCTGTCCTGCAA |
| | | GGCGTCTGGCTACACATTCACCACT |
| | | TACTGGATGCACTGGGTGAGGCAG |
| | | AGGCCTGGACAAGGCCTTGAGTGG |
| | | ATTGGAAATATTTATCCTGGTACTG |
| | | GTGGTTCTAACTTCGATGAGAAGTT |
| | | CAAAAACAGGACCTCACTGACTGT |
| | | AGACACATCCTCCACCACAGCCTA |
| | | CATGCACCTCGCCAGCCTGACATCT |
| | | GAGGACTCTGCGGTCTATTACTGTA |
| | | CAAGATGGACTACTGGGACGGGAG |
| | | CTTATTGGGGCCAGGGCACCACCG |
| | | TGACCGTGTCCTCC |
| SEQ ID NO: 156 | HC | QVQLQQPGSELVRPGASVKLS |
| | | CKASGYTFTTYWMHWVRQRPGQGL |
| | | EWIGNIYPGTGGSNFDEKFKNRTSLT |
| | | VDTSSTTAYMHLASLTSEDSAVYYC |
| | | TRWTTGTGAYWGQGTTVTVSSAST |
| | | KGPSVFPLAPCSRSTSESTAALGCLV |
| | | KDYFPEPVTVSWNSGALTSGVHTFP |
| | | AVLQSSGLYSLSSVVTVPSSSLGTKT |
| | | YTCNVDHKPSNTKVDKRVESKYGPP |
| | | CPPCPAPEFLGGPSVFLFPPKPKDTL |
| | | MISRTPEVTCVVVDVSQEDPEVQFN |
| | | WYVDGVEVHNAKTKPREEQFNSTY |
| | | RVVSVLTVLHQDWLNGKEYKCKVS |
| | | NKGLPSSIEKTISKAKGQPREPQVYT |
| | | LPPSQEEMTKNQVSLTCLVKGFYPSD |
| | | IAVEWESNGQPENNYKTTPPVLDSD |
| | | GSFFLYSRLTVDKSRWQEGNVFSCS |
| | | VMHEALHNHYTQKSLSLSLGK |
| SEQ ID NO: 157 | DNA HC | CAGGTCCAGCTGCAGCAGCC |
| | | TGGGTCTGAGCTGGTGAGGCCTGG |
| | | AGCTTCAGTGAAGCTGTCCTGCAA |
| | | GGCGTCTGGCTACACATTCACCACT |
| | | TACTGGATGCACTGGGTGAGGCAG |
| | | AGGCCTGGACAAGGCCTTGAGTGG |
| | | ATTGGAAATATTTATCCTGGTACTG |
| | | GTGGTTCTAACTTCGATGAGAAGTT |
| | | CAAAAACAGGACCTCACTGACTGT |
| | | AGACACATCCTCCACCACAGCCTA |
| | | CATGCACCTCGCCAGCCTGACATCT |
| | | GAGGACTCTGCGGTCTATTACTGTA |
| | | CAAGATGGACTACTGGGACGGGAG |
| | | CTTATTGGGGCCAGGGCACCACCG |
| | | TGACCGTGTCCTCCGCTTCCACCAA |
| | | GGGCCCATCCGTCTTCCCCCTGGCG |
| | | CCCTGCTCCAGGAGCACCTCCGAG |
| | | AGCACAGCCGCCCTGGGCTGCCTG |
| | | GTCAAGGACTACTTCCCCGAACCG |
| | | GTGACGGTGTCGTGGAACTCAGGC |
| | | GCCCTGACCAGCGGCGTGCACACC |
| | | TTCCCGGCTGTCCTACAGTCCTCAG |
| | | GACTCTACTCCCTCAGCAGCGTGGT |
| | | GACCGTGCCCTCCAGCAGCTTGGG |
| | | CACGAAGACCTACACCTGCAACGT |
| | | AGATCACAAGCCCAGCAACACCAA |
| | | GGTGGACAAGAGAGTTGAGTCCAA |
| | | ATATGGTCCCCCATGCCCACCGTGC |
| | | CCAGCACCTGAGTTCCTGGGGGGA |
| | | CCATCAGTCTTCCTGTTCCCCCCAA |
| | | AACCCAAGGACACTCTCATGATCT |
| | | CCCGGACCCCTGAGGTCACGTGCG |
| | | TGGTGGTGGACGTGAGCCAGGAAG |
| | | ACCCCGAGGTCCAGTTCAACTGGT |
| | | ACGTGGATGGCGTGGAGGTGCATA |
| | | ATGCCAAGACAAAGCCGCGGGAGG |
| | | AGCAGTTCAACAGCACGTACCGTG |
| | | TGGTCAGCGTCCTCACCGTCCTGCA |
| | | CCAGGACTGGCTGAACGGCAAGGA |
| | | GTACAAGTGCAAGGTGTCCAACAA |
| | | AGGCCTCCCGTCCTCCATCGAGAA |
| | | AACCATCTCCAAAGCCAAAGGGCA |
| | | GCCCCGAGAGCCACAGGTGTACAC |
| | | CCTGCCCCCATCCCAGGAGGAGAT |
| | | GACCAAGAACCAGGTCAGCCTGAC |
| | | CTGCCTGGTCAAAGGCTTCTACCCC |
| | | AGCGACATCGCCGTGGAGTGGGAG |
| | | AGCAATGGGCAGCCGGAGAACAAC |
| | | TACAAGACCACGCCTCCCGTGCTG |
| | | GACTCCGACGGCTCCTTCTTCCTCT |
| | | ACAGCAGGCTAACCGTGGACAAGA |
| | | GCAGGTGGCAGGAGGGGAATGTCT |
| | | TCTCATGCTCCGTGATGCATGAGGC |
| | | TCTGCACAACCACTACACACAGAA |
| | | GAGCCTCTCCCTGTCTCTGGGTAAA |
| SEQ ID NO: 158 | VH | QVQLQQSGSELVRPGASVKLS |
| | | CKASGYTFTTYWMHWVRQRPGQGL |
| | | EWIGNIYPGTGGSNFDEKFKNRTSLT |
| | | VDTSSTTAYMHLASLTSEDSAVYYC |
| | | TRWTTGTGAYWGQGTTVTVSS |
| SEQ ID NO: 159 | DNA VH | CAGGTCCAGCTGCAGCAGTC |
| | | TGGGTCTGAGCTGGTGAGGCCTGG |
| | | AGCTTCAGTGAAGCTGTCCTGCAA |
| | | GGCGTCTGGCTACACATTCACCACT |
| | | TACTGGATGCACTGGGTGAGGCAG |
| | | AGGCCTGGACAAGGCCTTGAGTGG |
| | | ATTGGAAATATTTATCCTGGTACTG |
| | | GTGGTTCTAACTTCGATGAGAAGTT |
| | | CAAAAACAGGACCTCACTGACTGT |
| | | AGACACATCCTCCACCACAGCCTA |
| | | CATGCACCTCGCCAGCCTGACATCT |
| | | GAGGACTCTGCGGTCTATTACTGTA |
| | | CAAGATGGACTACTGGGACGGGAG |
| | | CTTATTGGGGCCAGGGCACCACCG |
| | | TGACCGTGTCCTCC |
| SEQ ID NO: 160 | HC | QVQLQQSGSELVRPGASVKLS |
| | | CKASGYTFTTYWMHWVRQRPGQGL |
| | | EWIGNIYPGTGGSNFDEKFKNRTSLT |
| | | VDTSSTTAYMHLASLTSEDSAVYYC |
| | | TRWTTGTGAYWGQGTTVTVSSAST |
| | | KGPSVFPLAPCSRSTSESTAALGCLV |
| | | KDYFPEPVTVSWNSGALTSGVHTFP |
| | | AVLQSSGLYSLSSVVTVPSSSLGTKT |
| | | YTCNVDHKPSNTKVDKRVESKYGPP |
| | | CPPCPAPEFLGGPSVFLFPPKPKDTL |
| | | MISRTPEVTCVVVDVSQEDPEVQFN |
| | | WYVDGVEVHNAKTKPREEQFNSTY |
| | | RVVSVLTVLHQDWLNGKEYKCKVS |
| | | NKGLPSSIEKTISKAKGQPREPQVYT |
| | | LPPSQEEMTKNQVSLTCLVKGFYPSD |
| | | IAVEWESNGQPENNYKTTPPVLDSD |
| | | GSFFLYSRLTVDKSRWQEGNVFSCS |
| | | VMHEALHNHYTQKSLSLSLGK |
| SEQ ID NO: 161 | DNA HC | CAGGTCCAGCTGCAGCAGTC |
| | | TGGGTCTGAGCTGGTGAGGCCTGG |
| | | AGCTTCAGTGAAGCTGTCCTGCAA |
| | | GGCGTCTGGCTACACATTCACCACT |
| | | TACTGGATGCACTGGGTGAGGCAG |
| | | AGGCCTGGACAAGGCCTTGAGTGG |
| | | ATTGGAAATATTTATCCTGGTACTG |
| | | GTGGTTCTAACTTCGATGAGAAGTT |
| | | CAAAAACAGGACCTCACTGACTGT |
| | | AGACACATCCTCCACCACAGCCTA |
| | | CATGCACCTCGCCAGCCTGACATCT |
| | | GAGGACTCTGCGGTCTATTACTGTA |
| | | CAAGATGGACTACTGGGACGGGAG |
| | | CTTATTGGGGCCAGGGCACCACCG |
| | | TGACCGTGTCCTCCGCTTCCACCAA |
| | | GGGCCCATCCGTCTTCCCCCTGGCG |
| | | CCCTGCTCCAGGAGCACCTCCGAG |
| | | AGCACAGCCGCCCTGGGCTGCCTG |
| | | GTCAAGGACTACTTCCCCGAACCG |
| | | GTGACGGTGTCGTGGAACTCAGGC |
| | | GCCCTGACCAGCGGCGTGCACACC |
| | | TTCCCGGCTGTCCTACAGTCCTCAG |
| | | GACTCTACTCCCTCAGCAGCGTGGT |
| | | GACCGTGCCCTCCAGCAGCTTGGG |
| | | CACGAAGACCTACACCTGCAACGT |
| | | AGATCACAAGCCCAGCAACACCAA |
| | | GGTGGACAAGAGAGTTGAGTCCAA |
| | | ATATGGTCCCCCATGCCCACCGTGC |
| | | CCAGCACCTGAGTTCCTGGGGGGA |
| | | CCATCAGTCTTCCTGTTCCCCCCAA |
| | | AACCCAAGGACACTCTCATGATCT |
| | | CCCGGACCCCTGAGGTCACGTGCG |
| | | TGGTGGTGGACGTGAGCCAGGAAG |
| | | ACCCCGAGGTCCAGTTCAACTGGT |
| | | ACGTGGATGGCGTGGAGGTGCATA |
| | | ATGCCAAGACAAAGCCGCGGGAGG |
| | | AGCAGTTCAACAGCACGTACCGTG |
| | | TGGTCAGCGTCCTCACCGTCCTGCA |
| | | CCAGGACTGGCTGAACGGCAAGGA |
| | | GTACAAGTGCAAGGTGTCCAACAA |
| | | AGGCCTCCCGTCCTCCATCGAGAA |
| | | AACCATCTCCAAAGCCAAAGGGCA |
| | | GCCCCGAGAGCCACAGGTGTACAC |
| | | CCTGCCCCCATCCCAGGAGGAGAT |
| | | GACCAAGAACCAGGTCAGCCTGAC |
| | | CTGCCTGGTCAAAGGCTTCTACCCC |
| | | AGCGACATCGCCGTGGAGTGGGAG |
| | | AGCAATGGGCAGCCGGAGAACAAC |
| | | TACAAGACCACGCCTCCCGTGCTG |
| | | GACTCCGACGGCTCCTTCTTCCTCT |
| | | ACAGCAGGCTAACCGTGGACAAGA |
| | | GCAGGTGGCAGGAGGGGAATGTCT |
| | | TCTCATGCTCCGTGATGCATGAGGC |
| | | TCTGCACAACCACTACACACAGAA |
| | | GAGCCTCTCCCTGTCTCTGGGTAAA |
| BAP049-chi-Y LC | | |
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 | LCDR1 | SQSLLDSGNQKNF |
| (Chothia) | | |
| SEQ ID NO: 150 | LCDR2 | WAS |
| (Chothia) | | |
| SEQ ID NO: 167 | LCDR3 | DYSYPY |
| (Chothia) | | |
| SEQ ID NO: 168 | VL | DIVMTQSPSSLTVTAGEKVTM |
| | | SCKSSQSLLDSGNQKNFLTWYQQKP |
| | | GQPPKLLIFWASTRESGVPDRFTGSG |
| | | SVTDFTLTISSVQAEDLAVYYCQND |
| | | YSYPYTFGQGTKVEIK |
| SEQ ID NO: 169 | DNA VL | GACATTGTGATGACCCAGTC |
| | | TCCATCCTCCCTGACTGTGACAGCA |
| | | GGAGAGAAGGTCACTATGAGCTGC |
| | | AAGTCCAGTCAGAGTCTGTTAGAC |
| | | AGTGGAAATCAAAAGAACTTCTTG |
| | | ACCTGGTACCAGCAGAAACCAGGG |
| | | CAGCCTCCTAAACTGTTGATCTTCT |
| | | GGGCATCCACTAGGGAATCTGGGG |
| | | TCCCTGATCGCTTCACAGGCAGTGG |
| | | ATCTGTAACAGATTTCACTCTCACC |
| | | ATCAGCAGTGTGCAGGCTGAAGAC |
| | | CTGGCAGTTTATTACTGTCAGAATG |
| | | ATTATAGTTATCCGTACACGTTCGG |
| | | CCAAGGGACCAAGGTGGAAATCAA |
| | | A |
| SEQ ID NO: 170 | LC | DIVMTQSPSSLTVTAGEKVTM |
| | | SCKSSQSLLDSGNQKNFLTWYQQKP |
| | | GQPPKLLIFWASTRESGVPDRFTGSG |
| | | SVTDFTLTISSVQAEDLAVYYCQND |
| | | YSYPYTFGQGTKVEIKRTVAAPSVFI |
| | | FPPSDEQLKSGTASVVCLLNNFYPRE |
| | | AKVQWKVDNALQSGNSQESVTEQD |
| | | SKDSTYSLSSTLTLSKADYEKHKVY |
| | | ACEVTHQGLSSPVTKSFNRGEC |
| SEQ ID NO: 171 | DNA LC | GACATTGTGATGACCCAGTC |
| | | TCCATCCTCCCTGACTGTGACAGCA |
| | | GGAGAGAAGGTCACTATGAGCTGC |
| | | AAGTCCAGTCAGAGTCTGTTAGAC |
| | | AGTGGAAATCAAAAGAACTTCTTG |
| | | ACCTGGTACCAGCAGAAACCAGGG |
| | | CAGCCTCCTAAACTGTTGATCTTCT |
| | | GGGCATCCACTAGGGAATCTGGGG |
| | | TCCCTGATCGCTTCACAGGCAGTGG |
| | | ATCTGTAACAGATTTCACTCTCACC |
| | | ATCAGCAGTGTGCAGGCTGAAGAC |
| | | CTGGCAGTTTATTACTGTCAGAATG |
| | | ATTATAGTTATCCGTACACGTTCGG |
| | | CCAAGGGACCAAGGTGGAAATCAA |
| | | ACGTACGGTGGCTGCACCATCTGTC |
| | | TTCATCTTCCCGCCATCTGATGAGC |
| | | AGTTGAAATCTGGAACTGCCTCTGT |
| | | TGTGTGCCTGCTGAATAACTTCTAT |
| | | CCCAGAGAGGCCAAAGTACAGTGG |
| | | AAGGTGGATAACGCCCTCCAATCG |
| | | GGTAACTCCCAGGAGAGTGTCACA |
| | | GAGCAGGACAGCAAGGACAGCACC |
| | | TACAGCCTCAGCAGCACCCTGACG |
| | | CTGAGCAAAGCAGACTACGAGAAA |
| | | CACAAAGTCTACGCCTGCGAAGTC |
| | | ACCCATCAGGGCCTGAGCTCGCCC |
| | | GTCACAAAGAGCTTCAACAGGGGA |
| | | GAGTGT |
| BAP049-hum01 HC | | |
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 | HCDR1 | GYTFTTY |
| (Chothia) | | |
| SEQ ID NO: 141 | HCDR2 | YPGTGG |
| (Chothia) | | |
| SEQ ID NO: 139 | HCDR3 | WTTGTGAY |
| (Chothia) | | |
| SEQ ID NO: 172 | VH | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSS |
| SEQ ID NO: 173 | DNA VH | GAAGTGCAGCTGGTGCAGTC |
| | | TGGAGCAGAGGTGAAAAAGCCCGG |
| | | GGAGTCTCTGAGGATCTCCTGTAA |
| | | GGGTTCTGGCTACACATTCACCACT |
| | | TACTGGATGCACTGGGTGCGACAG |
| | | GCCACTGGACAAGGGCTTGAGTGG |
| | | ATGGGTAATATTTATCCTGGTACTG |
| | | GTGGTTCTAACTTCGATGAGAAGTT |
| | | CAAGAACAGAGTCACGATTACCGC |
| | | GGACAAATCCACGAGCACAGCCTA |
| | | CATGGAGCTGAGCAGCCTGAGATC |
| | | TGAGGACACGGCCGTGTATTACTG |
| | | TACAAGATGGACTACTGGGACGGG |
| | | AGCTTATTGGGGCCAGGGCACCAC |
| | | CGTGACCGTGTCCTCC |
| SEQ ID NO: 174 | HC | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSSAS |
| | | TKGPSVFPLAPCSRSTSESTAALGCL |
| | | VKDYFPEPVTVSWNSGALTSGVHTF |
| | | PAVLQSSGLYSLSSVVTVPSSSLGTK |
| | | TYTCNVDHKPSNTKVDKRVESKYGP |
| | | PCPPCPAPEFLGGPSVFLFPPKPKDTL |
| | | MISRTPEVTCVVVDVSQEDPEVQFN |
| | | WYVDGVEVHNAKTKPREEQFNSTY |
| | | RVVSVLTVLHQDWLNGKEYKCKVS |
| | | NKGLPSSIEKTISKAKGQPREPQVYT |
| | | LPPSQEEMTKNQVSLTCLVKGFYPSD |
| | | IAVEWESNGQPENNYKTTPPVLDSD |
| | | GSFFLYSRLTVDKSRWQEGNVFSCS |
| | | VMHEALHNHYTQKSLSLSLGK |
| SEQ ID NO: 175 | DNA HC | GAAGTGCAGCTGGTGCAGTC |
| | | TGGAGCAGAGGTGAAAAAGCCCGG |
| | | GGAGTCTCTGAGGATCTCCTGTAA |
| | | GGGTTCTGGCTACACATTCACCACT |
| | | TACTGGATGCACTGGGTGCGACAG |
| | | GCCACTGGACAAGGGCTTGAGTGG |
| | | ATGGGTAATATTTATCCTGGTACTG |
| | | GTGGTTCTAACTTCGATGAGAAGTT |
| | | CAAGAACAGAGTCACGATTACCGC |
| | | GGACAAATCCACGAGCACAGCCTA |
| | | CATGGAGCTGAGCAGCCTGAGATC |
| | | TGAGGACACGGCCGTGTATTACTG |
| | | TACAAGATGGACTACTGGGACGGG |
| | | AGCTTATTGGGGCCAGGGCACCAC |
| | | CGTGACCGTGTCCTCCGCTTCCACC |
| | | AAGGGCCCATCCGTCTTCCCCCTGG |
| | | CGCCCTGCTCCAGGAGCACCTCCG |
| | | AGAGCACAGCCGCCCTGGGCTGCC |
| | | TGGTCAAGGACTACTTCCCCGAAC |
| | | CGGTGACGGTGTCGTGGAACTCAG |
| | | GCGCCCTGACCAGCGGCGTGCACA |
| | | CCTTCCCGGCTGTCCTACAGTCCTC |
| | | AGGACTCTACTCCCTCAGCAGCGT |
| | | GGTGACCGTGCCCTCCAGCAGCTT |
| | | GGGCACGAAGACCTACACCTGCAA |
| | | CGTAGATCACAAGCCCAGCAACAC |
| | | CAAGGTGGACAAGAGAGTTGAGTC |
| | | CAAATATGGTCCCCCATGCCCACC |
| | | GTGCCCAGCACCTGAGTTCCTGGG |
| | | GGGACCATCAGTCTTCCTGTTCCCC |
| | | CCAAAACCCAAGGACACTCTCATG |
| | | ATCTCCCGGACCCCTGAGGTCACGT |
| | | GCGTGGTGGTGGACGTGAGCCAGG |
| | | AAGACCCCGAGGTCCAGTTCAACT |
| | | GGTACGTGGATGGCGTGGAGGTGC |
| | | ATAATGCCAAGACAAAGCCGCGGG |
| | | AGGAGCAGTTCAACAGCACGTACC |
| | | GTGTGGTCAGCGTCCTCACCGTCCT |
| | | GCACCAGGACTGGCTGAACGGCAA |
| | | GGAGTACAAGTGCAAGGTGTCCAA |
| | | CAAAGGCCTCCCGTCCTCCATCGA |
| | | GAAAACCATCTCCAAAGCCAAAGG |
| | | GCAGCCCCGAGAGCCACAGGTGTA |
| | | CACCCTGCCCCCATCCCAGGAGGA |
| | | GATGACCAAGAACCAGGTCAGCCT |
| | | GACCTGCCTGGTCAAAGGCTTCTAC |
| | | CCCAGCGACATCGCCGTGGAGTGG |
| | | GAGAGCAATGGGCAGCCGGAGAAC |
| | | AACTACAAGACCACGCCTCCCGTG |
| | | CTGGACTCCGACGGCTCCTTCTTCC |
| | | TCTACAGCAGGCTAACCGTGGACA |
| | | AGAGCAGGTGGCAGGAGGGGAAT |
| | | GTCTTCTCATGCTCCGTGATGCATG |
| | | AGGCTCTGCACAACCACTACACAC |
| | | AGAAGAGCCTCTCCCTGTCTCTGGG |
| | | TAAA |
| BAP049-hum01 LC | | |
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 | LCDR1 | SQSLLDSGNQKNF |
| (Chothia) | | |
| SEQ ID NO: 150 | LCDR2 | WAS |
| (Chothia) | | |
| SEQ ID NO: 167 | LCDR3 | DYSYPY |
| (Chothia) | | |
| SEQ ID NO: 176 | VL | EIVLTQSPATLSLSPGERATLS |
| | | CKSSQSLLDSGNQKNFLTWYQQKPG |
| | | QAPRLLIYWASTRESGVPSRFSGSGS |
| | | GTEFTLTISSLQPDDFATYYCQNDYS |
| | | YPYTFGQGTKVEIK |
| SEQ ID NO: 177 | DNA VL | GAAATTGTGTTGACACAGTC |
| | | TCCAGCCACCCTGTCTTTGTCTCCA |
| | | GGGGAAAGAGCCACCCTCTCCTGC |
| | | AAGTCCAGTCAGAGTCTGTTAGAC |
| | | AGTGGAAATCAAAAGAACTTCTTG |
| | | ACCTGGTACCAGCAGAAACCTGGC |
| | | CAGGCTCCCAGGCTCCTCATCTATT |
| | | GGGCATCCACTAGGGAATCTGGGG |
| | | TCCCATCAAGGTTCAGCGGCAGTG |
| | | GATCTGGGACAGAATTCACTCTCA |
| | | CCATCAGCAGCCTGCAGCCTGATG |
| | | ATTTTGCAACTTATTACTGTCAGAA |
| | | TGATTATAGTTATCCGTACACGTTC |
| | | GGCCAAGGGACCAAGGTGGAAATC |
| | | AAA |
| SEQ ID NO: 178 | LC | EIVLTQSPATLSLSPGERATLS |
| | | CKSSQSLLDSGNQKNFLTWYQQKPG |
| | | QAPRLLIYWASTRESGVPSRFSGSGS |
| | | GTEFTLTISSLQPDDFATYYCQNDYS |
| | | YPYTFGQGTKVEIKRTVAAPSVFIFPP |
| | | SDEQLKSGTASVVCLLNNFYPREAK |
| | | VQWKVDNALQSGNSQESVTEQDSK |
| | | DSTYSLSSTLTLSKADYEKHKVYAC |
| | | EVTHQGLSSPVTKSFNRGEC |
| SEQ ID NO: 179 | DNA LC | GAAATTGTGTTGACACAGTC |
| | | TCCAGCCACCCTGTCTTTGTCTCCA |
| | | GGGGAAAGAGCCACCCTCTCCTGC |
| | | AAGTCCAGTCAGAGTCTGTTAGAC |
| | | AGTGGAAATCAAAAGAACTTCTTG |
| | | ACCTGGTACCAGCAGAAACCTGGC |
| | | CAGGCTCCCAGGCTCCTCATCTATT |
| | | GGGCATCCACTAGGGAATCTGGGG |
| | | TCCCATCAAGGTTCAGCGGCAGTG |
| | | GATCTGGGACAGAATTCACTCTCA |
| | | CCATCAGCAGCCTGCAGCCTGATG |
| | | ATTTTGCAACTTATTACTGTCAGAA |
| | | TGATTATAGTTATCCGTACACGTTC |
| | | GGCCAAGGGACCAAGGTGGAAATC |
| | | AAACGTACGGTGGCTGCACCATCT |
| | | GTCTTCATCTTCCCGCCATCTGATG |
| | | AGCAGTTGAAATCTGGAACTGCCT |
| | | CTGTTGTGTGCCTGCTGAATAACTT |
| | | CTATCCCAGAGAGGCCAAAGTACA |
| | | GTGGAAGGTGGATAACGCCCTCCA |
| | | ATCGGGTAACTCCCAGGAGAGTGT |
| | | CACAGAGCAGGACAGCAAGGACA |
| | | GCACCTACAGCCTCAGCAGCACCC |
| | | TGACGCTGAGCAAAGCAGACTACG |
| | | AGAAACACAAAGTCTACGCCTGCG |
| | | AAGTCACCCATCAGGGCCTGAGCT |
| | | CGCCCGTCACAAAGAGCTTCAACA |
| | | GGGGAGAGTGT |
| BAP049-hum02 HC | | |
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 | HCDR1 | GYTFTTY |
| (Chothia) | | |
| SEQ ID NO: 141 | HCDR2 | YPGTGG |
| (Chothia) | | |
| SEQ ID NO: 139 | HCDR3 | WTTGTGAY |
| (Chothia) | | |
| SEQ ID NO: 172 | VH | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSS |
| SEQ ID NO: 173 | DNA VH | GAAGTGCAGCTGGTGCAGTC |
| | | TGGAGCAGAGGTGAAAAAGCCCGG |
| | | GGAGTCTCTGAGGATCTCCTGTAA |
| | | GGGTTCTGGCTACACATTCACCACT |
| | | TACTGGATGCACTGGGTGCGACAG |
| | | GCCACTGGACAAGGGCTTGAGTGG |
| | | ATGGGTAATATTTATCCTGGTACTG |
| | | GTGGTTCTAACTTCGATGAGAAGTT |
| | | CAAGAACAGAGTCACGATTACCGC |
| | | GGACAAATCCACGAGCACAGCCTA |
| | | CATGGAGCTGAGCAGCCTGAGATC |
| | | TGAGGACACGGCCGTGTATTACTG |
| | | TACAAGATGGACTACTGGGACGGG |
| | | AGCTTATTGGGGCCAGGGCACCAC |
| | | CGTGACCGTGTCCTCC |
| SEQ ID NO: 174 | HC | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSSAS |
| | | TKGPSVFPLAPCSRSTSESTAALGCL |
| | | VKDYFPEPVTVSWNSGALTSGVHTF |
| | | PAVLQSSGLYSLSSVVTVPSSSLGTK |
| | | TYTCNVDHKPSNTKVDKRVESKYGP |
| | | PCPPCPAPEFLGGPSVFLFPPKPKDTL |
| | | MISRTPEVTCVVVDVSQEDPEVQFN |
| | | WYVDGVEVHNAKTKPREEQFNSTY |
| | | RVVSVLTVLHQDWLNGKEYKCKVS |
| | | NKGLPSSIEKTISKAKGQPREPQVYT |
| | | LPPSQEEMTKNQVSLTCLVKGFYPSD |
| | | IAVEWESNGQPENNYKTTPPVLDSD |
| | | GSFFLYSRLTVDKSRWQEGNVFSCS |
| | | VMHEALHNHYTQKSLSLSLGK |
| SEQ ID NO: 175 | DNA HC | GAAGTGCAGCTGGTGCAGTC |
| | | TGGAGCAGAGGTGAAAAAGCCCGG |
| | | GGAGTCTCTGAGGATCTCCTGTAA |
| | | GGGTTCTGGCTACACATTCACCACT |
| | | TACTGGATGCACTGGGTGCGACAG |
| | | GCCACTGGACAAGGGCTTGAGTGG |
| | | ATGGGTAATATTTATCCTGGTACTG |
| | | GTGGTTCTAACTTCGATGAGAAGTT |
| | | CAAGAACAGAGTCACGATTACCGC |
| | | GGACAAATCCACGAGCACAGCCTA |
| | | CATGGAGCTGAGCAGCCTGAGATC |
| | | TGAGGACACGGCCGTGTATTACTG |
| | | TACAAGATGGACTACTGGGACGGG |
| | | AGCTTATTGGGGCCAGGGCACCAC |
| | | CGTGACCGTGTCCTCCGCTTCCACC |
| | | AAGGGCCCATCCGTCTTCCCCCTGG |
| | | CGCCCTGCTCCAGGAGCACCTCCG |
| | | AGAGCACAGCCGCCCTGGGCTGCC |
| | | TGGTCAAGGACTACTTCCCCGAAC |
| | | CGGTGACGGTGTCGTGGAACTCAG |
| | | GCGCCCTGACCAGCGGCGTGCACA |
| | | CCTTCCCGGCTGTCCTACAGTCCTC |
| | | AGGACTCTACTCCCTCAGCAGCGT |
| | | GGTGACCGTGCCCTCCAGCAGCTT |
| | | GGGCACGAAGACCTACACCTGCAA |
| | | CGTAGATCACAAGCCCAGCAACAC |
| | | CAAGGTGGACAAGAGAGTTGAGTC |
| | | CAAATATGGTCCCCCATGCCCACC |
| | | GTGCCCAGCACCTGAGTTCCTGGG |
| | | GGGACCATCAGTCTTCCTGTTCCCC |
| | | CCAAAACCCAAGGACACTCTCATG |
| | | ATCTCCCGGACCCCTGAGGTCACGT |
| | | GCGTGGTGGTGGACGTGAGCCAGG |
| | | AAGACCCCGAGGTCCAGTTCAACT |
| | | GGTACGTGGATGGCGTGGAGGTGC |
| | | ATAATGCCAAGACAAAGCCGCGGG |
| | | AGGAGCAGTTCAACAGCACGTACC |
| | | GTGTGGTCAGCGTCCTCACCGTCCT |
| | | GCACCAGGACTGGCTGAACGGCAA |
| | | GGAGTACAAGTGCAAGGTGTCCAA |
| | | CAAAGGCCTCCCGTCCTCCATCGA |
| | | GAAAACCATCTCCAAAGCCAAAGG |
| | | GCAGCCCCGAGAGCCACAGGTGTA |
| | | CACCCTGCCCCCATCCCAGGAGGA |
| | | GATGACCAAGAACCAGGTCAGCCT |
| | | GACCTGCCTGGTCAAAGGCTTCTAC |
| | | CCCAGCGACATCGCCGTGGAGTGG |
| | | GAGAGCAATGGGCAGCCGGAGAAC |
| | | AACTACAAGACCACGCCTCCCGTG |
| | | CTGGACTCCGACGGCTCCTTCTTCC |
| | | TCTACAGCAGGCTAACCGTGGACA |
| | | AGAGCAGGTGGCAGGAGGGGAAT |
| | | GTCTTCTCATGCTCCGTGATGCATG |
| | | AGGCTCTGCACAACCACTACACAC |
| | | AGAAGAGCCTCTCCCTGTCTCTGGG |
| | | TAAA |
| BAP049-hum02 LC | | |
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPT |
| SEQ ID NO: 149 | LCDR1 | SQSLLDSGNQKNF |
| (Chothia) | | |
| SEQ ID NO: 150 | LCDR2 | WAS |
| (Chothia) | | |
| SEQ ID NO: 167 | LCDR3 | DYSYPY |
| (Chothia) | | |
| SEQ ID NO: 180 | VL | DIQMTQSPSSLSASVGDRVTIT |
| | | CKSSQSLLDSGNQKNFLTWYQQKPG |
| | | QAPRLLIYWASTRESGIPPRFSGSGY |
| | | GTDFTLTINNIESEDAAYYFCQNDYS |
| | | YPYTFGQGTKVEIK |
| SEQ ID NO: 181 | DNA VL | GACATCCAGATGACCCAGTC |
| | | TCCATCCTCCCTGTCTGCATCTGTA |
| | | GGAGACAGAGTCACCATCACTTGC |
| | | AAGTCCAGTCAGAGTCTGTTAGAC |
| | | AGTGGAAATCAAAAGAACTTCTTG |
| | | ACCTGGTACCAGCAGAAACCTGGC |
| | | CAGGCTCCCAGGCTCCTCATCTATT |
| | | GGGCATCCACTAGGGAATCTGGGA |
| | | TCCCACCTCGATTCAGTGGCAGCG |
| | | GGTATGGAACAGATTTTACCCTCAC |
| | | AATTAATAACATAGAATCTGAGGA |
| | | TGCTGCATATTACTTCTGTCAGAAT |
| | | GATTATAGTTATCCGTACACGTTCG |
| | | GCCAAGGGACCAAGGTGGAAATCA |
| | | AA |
| SEQ ID NO: 182 | LC | DIQMTQSPSSLSASVGDRVTIT |
| | | CKSSQSLLDSGNQKNFLTWYQQKPG |
| | | QAPRLLIYWASTRESGIPPRFSGSGY |
| | | GTDFTLTINNIESEDAAYYFCQNDYS |
| | | YPYTFGQGTKVEIKRTVAAPSVFIFPP |
| | | SDEQLKSGTASVVCLLNNFYPREAK |
| | | VQWKVDNALQSGNSQESVTEQDSK |
| | | DSTYSLSSTLTLSKADYEKHKVYAC |
| | | EVTHQGLSSPVTKSFNRGEC |
| SEQ ID NO: 183 | DNA LC | GACATCCAGATGACCCAGTC |
| | | TCCATCCTCCCTGTCTGCATCTGTA |
| | | GGAGACAGAGTCACCATCACTTGC |
| | | AAGTCCAGTCAGAGTCTGTTAGAC |
| | | AGTGGAAATCAAAAGAACTTCTTG |
| | | ACCTGGTACCAGCAGAAACCTGGC |
| | | CAGGCTCCCAGGCTCCTCATCTATT |
| | | GGGCATCCACTAGGGAATCTGGGA |
| | | TCCCACCTCGATTCAGTGGCAGCG |
| | | GGTATGGAACAGATTTTACCCTCAC |
| | | AATTAATAACATAGAATCTGAGGA |
| | | TGCTGCATATTACTTCTGTCAGAAT |
| | | GATTATAGTTATCCGTACACGTTCG |
| | | GCCAAGGGACCAAGGTGGAAATCA |
| | | AACGTACGGTGGCTGCACCATCTG |
| | | TCTTCATCTTCCCGCCATCTGATGA |
| | | GCAGTTGAAATCTGGAACTGCCTCT |
| | | GTTGTGTGCCTGCTGAATAACTTCT |
| | | ATCCCAGAGAGGCCAAAGTACAGT |
| | | GGAAGGTGGATAACGCCCTCCAAT |
| | | CGGGTAACTCCCAGGAGAGTGTCA |
| | | CAGAGCAGGACAGCAAGGACAGC |
| | | ACCTACAGCCTCAGCAGCACCCTG |
| | | ACGCTGAGCAAAGCAGACTACGAG |
| | | AAACACAAAGTCTACGCCTGCGAA |
| | | GTCACCCATCAGGGCCTGAGCTCG |
| | | CCCGTCACAAAGAGCTTCAACAGG |
| | | GGAGAGTGT |
| BAP049-hum03 HC | | |
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 | HCDR1 | GYTFTTY |
| (Chothia) | | |
| SEQ ID NO: 141 | HCDR2 | YPGTGG |
| (Chothia) | | |
| SEQ ID NO: 139 | HCDR3 | WTTGTGAY |
| (Chothia) | | |
| SEQ ID NO: 184 | VH | EVQLVQSGAEVKKPGESLRIS |
| | | CKGSGYTFTTYWMHWIRQSPSRGLE |
| | | WLGNIYPGTGGSNFDEKFKNRFTISR |
| | | DNSKNTLYLQMNSLRAEDTAVYYC |
| | | TRWTTGTGAYWGQGTTVTVSS |
| SEQ ID NO: 185 | DNA VH | GAAGTGCAGCTGGTGCAGTC |
| | | TGGAGCAGAGGTGAAAAAGCCCGG |
| | | GGAGTCTCTGAGGATCTCCTGTAA |
| | | GGGTTCTGGCTACACATTCACCACT |
| | | TACTGGATGCACTGGATCAGGCAG |
| | | TCCCCATCGAGAGGCCTTGAGTGG |
| | | CTGGGTAATATTTATCCTGGTACTG |
| | | GTGGTTCTAACTTCGATGAGAAGTT |
| | | CAAGAACAGATTCACCATCTCCAG |
| | | AGACAATTCCAAGAACACGCTGTA |
| | | TCTTCAAATGAACAGCCTGAGAGC |
| | | CGAGGACACGGCCGTGTATTACTG |
| | | TACAAGATGGACTACTGGGACGGG |
| | | AGCTTATTGGGGCCAGGGCACCAC |
| | | CGTGACCGTGTCCTCC |
| SEQ ID NO: 186 | HC | EVQLVQSGAEVKKPGESLRIS |
| | | CKGSGYTFTTYWMHWIRQSPSRGLE |
| | | WLGNIYPGTGGSNFDEKFKNRFTISR |
| | | DNSKNTLYLQMNSLRAEDTAVYYC |
| | | TRWTTGTGAYWGQGTTVTVSSAST |
| | | KGPSVFPLAPCSRSTSESTAALGCLV |
| | | KDYFPEPVTVSWNSGALTSGVHTFP |
| | | AVLQSSGLYSLSSVVTVPSSSLGTKT |
| | | YTCNVDHKPSNTKVDKRVESKYGPP |
| | | CPPCPAPEFLGGPSVFLFPPKPKDTL |
| | | MISRTPEVTCVVVDVSQEDPEVQFN |
| | | WYVDGVEVHNAKTKPREEQFNSTY |
| | | RVVSVLTVLHQDWLNGKEYKCKVS |
| | | NKGLPSSIEKTISKAKGQPREPQVYT |
| | | LPPSQEEMTKNQVSLTCLVKGFYPSD |
| | | IAVEWESNGQPENNYKTTPPVLDSD |
| | | GSFFLYSRLTVDKSRWQEGNVFSCS |
| | | VMHEALHNHYTQKSLSLSLGK |
| SEQ ID NO: 187 | DNA HC | GAAGTGCAGCTGGTGCAGTC |
| | | TGGAGCAGAGGTGAAAAAGCCCGG |
| | | GGAGTCTCTGAGGATCTCCTGTAA |
| | | GGGTTCTGGCTACACATTCACCACT |
| | | TACTGGATGCACTGGATCAGGCAG |
| | | TCCCCATCGAGAGGCCTTGAGTGG |
| | | CTGGGTAATATTTATCCTGGTACTG |
| | | GTGGTTCTAACTTCGATGAGAAGTT |
| | | CAAGAACAGATTCACCATCTCCAG |
| | | AGACAATTCCAAGAACACGCTGTA |
| | | TCTTCAAATGAACAGCCTGAGAGC |
| | | CGAGGACACGGCCGTGTATTACTG |
| | | TACAAGATGGACTACTGGGACGGG |
| | | AGCTTATTGGGGCCAGGGCACCAC |
| | | CGTGACCGTGTCCTCCGCTTCCACC |
| | | AAGGGCCCATCCGTCTTCCCCCTGG |
| | | CGCCCTGCTCCAGGAGCACCTCCG |
| | | AGAGCACAGCCGCCCTGGGCTGCC |
| | | TGGTCAAGGACTACTTCCCCGAAC |
| | | CGGTGACGGTGTCGTGGAACTCAG |
| | | GCGCCCTGACCAGCGGCGTGCACA |
| | | CCTTCCCGGCTGTCCTACAGTCCTC |
| | | AGGACTCTACTCCCTCAGCAGCGT |
| | | GGTGACCGTGCCCTCCAGCAGCTT |
| | | GGGCACGAAGACCTACACCTGCAA |
| | | CGTAGATCACAAGCCCAGCAACAC |
| | | CAAGGTGGACAAGAGAGTTGAGTC |
| | | CAAATATGGTCCCCCATGCCCACC |
| | | GTGCCCAGCACCTGAGTTCCTGGG |
| | | GGGACCATCAGTCTTCCTGTTCCCC |
| | | CCAAAACCCAAGGACACTCTCATG |
| | | ATCTCCCGGACCCCTGAGGTCACGT |
| | | GCGTGGTGGTGGACGTGAGCCAGG |
| | | AAGACCCCGAGGTCCAGTTCAACT |
| | | GGTACGTGGATGGCGTGGAGGTGC |
| | | ATAATGCCAAGACAAAGCCGCGGG |
| | | AGGAGCAGTTCAACAGCACGTACC |
| | | GTGTGGTCAGCGTCCTCACCGTCCT |
| | | GCACCAGGACTGGCTGAACGGCAA |
| | | GGAGTACAAGTGCAAGGTGTCCAA |
| | | CAAAGGCCTCCCGTCCTCCATCGA |
| | | GAAAACCATCTCCAAAGCCAAAGG |
| | | GCAGCCCCGAGAGCCACAGGTGTA |
| | | CACCCTGCCCCCATCCCAGGAGGA |
| | | GATGACCAAGAACCAGGTCAGCCT |
| | | GACCTGCCTGGTCAAAGGCTTCTAC |
| | | CCCAGCGACATCGCCGTGGAGTGG |
| | | GAGAGCAATGGGCAGCCGGAGAAC |
| | | AACTACAAGACCACGCCTCCCGTG |
| | | CTGGACTCCGACGGCTCCTTCTTCC |
| | | TCTACAGCAGGCTAACCGTGGACA |
| | | AGAGCAGGTGGCAGGAGGGGAAT |
| | | GTCTTCTCATGCTCCGTGATGCATG |
| | | AGGCTCTGCACAACCACTACACAC |
| | | AGAAGAGCCTCTCCCTGTCTCTGGG |
| | | TAAA |
| BAP049-hum03 LC | | |
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 | LCDR1 | SQSLLDSGNQKNF |
| (Chothia) | | |
| SEQ ID NO: 150 | LCDR2 | WAS |
| (Chothia) | | |
| SEQ ID NO: 167 | LCDR3 | DYSYPY |
| (Chothia) | | |
| SEQ ID NO: 180 | VL | DIQMTQSPSSLSASVGDRVTIT |
| | | CKSSQSLLDSGNQKNFLTWYQQKPG |
| | | QAPRLLIYWASTRESGIPPRFSGSGY |
| | | GTDFTLTINNIESEDAAYYFCQNDYS |
| | | YPYTFGQGTKVEIK |
| SEQ ID NO: 181 | DNA VL | GACATCCAGATGACCCAGTC |
| | | TCCATCCTCCCTGTCTGCATCTGTA |
| | | GGAGACAGAGTCACCATCACTTGC |
| | | AAGTCCAGTCAGAGTCTGTTAGAC |
| | | AGTGGAAATCAAAAGAACTTCTTG |
| | | ACCTGGTACCAGCAGAAACCTGGC |
| | | CAGGCTCCCAGGCTCCTCATCTATT |
| | | GGGCATCCACTAGGGAATCTGGGA |
| | | TCCCACCTCGATTCAGTGGCAGCG |
| | | GGTATGGAACAGATTTTACCCTCAC |
| | | AATTAATAACATAGAATCTGAGGA |
| | | TGCTGCATATTACTTCTGTCAGAAT |
| | | GATTATAGTTATCCGTACACGTTCG |
| | | GCCAAGGGACCAAGGTGGAAATCA |
| | | AA |
| SEQ ID NO: 182 | LC | DIQMTQSPSSLSASVGDRVTIT |
| | | CKSSQSLLDSGNQKNFLTWYQQKPG |
| | | QAPRLLIYWASTRESGIPPRFSGSGY |
| | | GTDFTLTINNIESEDAAYYFCQNDYS |
| | | YPYTFGQGTKVEIKRTVAAPSVFIFPP |
| | | SDEQLKSGTASVVCLLNNFYPREAK |
| | | VQWKVDNALQSGNSQESVTEQDSK |
| | | DSTYSLSSTLTLSKADYEKHKVYAC |
| | | EVTHQGLSSPVTKSFNRGEC |
| SEQ ID NO: 183 | DNA LC | GACATCCAGATGACCCAGTC |
| | | TCCATCCTCCCTGTCTGCATCTGTA |
| | | GGAGACAGAGTCACCATCACTTGC |
| | | AAGTCCAGTCAGAGTCTGTTAGAC |
| | | AGTGGAAATCAAAAGAACTTCTTG |
| | | ACCTGGTACCAGCAGAAACCTGGC |
| | | CAGGCTCCCAGGCTCCTCATCTATT |
| | | GGGCATCCACTAGGGAATCTGGGA |
| | | TCCCACCTCGATTCAGTGGCAGCG |
| | | GGTATGGAACAGATTTTACCCTCAC |
| | | AATTAATAACATAGAATCTGAGGA |
| | | TGCTGCATATTACTTCTGTCAGAAT |
| | | GATTATAGTTATCCGTACACGTTCG |
| | | GCCAAGGGACCAAGGTGGAAATCA |
| | | AACGTACGGTGGCTGCACCATCTG |
| | | TCTTCATCTTCCCGCCATCTGATGA |
| | | GCAGTTGAAATCTGGAACTGCCTCT |
| | | GTTGTGTGCCTGCTGAATAACTTCT |
| | | ATCCCAGAGAGGCCAAAGTACAGT |
| | | GGAAGGTGGATAACGCCCTCCAAT |
| | | CGGGTAACTCCCAGGAGAGTGTCA |
| | | CAGAGCAGGACAGCAAGGACAGC |
| | | ACCTACAGCCTCAGCAGCACCCTG |
| | | ACGCTGAGCAAAGCAGACTACGAG |
| | | AAACACAAAGTCTACGCCTGCGAA |
| | | GTCACCCATCAGGGCCTGAGCTCG |
| | | CCCGTCACAAAGAGCTTCAACAGG |
| | | GGAGAGTGT |
| BAP049-hum04 HC | | |
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 | HCDR1 | GYTFTTY |
| (Chothia) | | |
| SEQ ID NO: 141 | HCDR2 | YPGTGG |
| (Chothia) | | |
| SEQ ID NO: 139 | HCDR3 | WTTGTGAY |
| (Chothia) | | |
| SEQ ID NO: 184 | VH | EVQLVQSGAEVKKPGESLRIS |
| | | CKGSGYTFTTYWMHWIRQSPSRGLE |
| | | WLGNIYPGTGGSNFDEKFKNRFTISR |
| | | DNSKNTLYLQMNSLRAEDTAVYYC |
| | | TRWTTGTGAYWGQGTTVTVSS |
| SEQ ID NO: 185 | DNA VH | GAAGTGCAGCTGGTGCAGTC |
| | | TGGAGCAGAGGTGAAAAAGCCCGG |
| | | GGAGTCTCTGAGGATCTCCTGTAA |
| | | GGGTTCTGGCTACACATTCACCACT |
| | | TACTGGATGCACTGGATCAGGCAG |
| | | TCCCCATCGAGAGGCCTTGAGTGG |
| | | CTGGGTAATATTTATCCTGGTACTG |
| | | GTGGTTCTAACTTCGATGAGAAGTT |
| | | CAAGAACAGATTCACCATCTCCAG |
| | | AGACAATTCCAAGAACACGCTGTA |
| | | TCTTCAAATGAACAGCCTGAGAGC |
| | | CGAGGACACGGCCGTGTATTACTG |
| | | TACAAGATGGACTACTGGGACGGG |
| | | AGCTTATTGGGGCCAGGGCACCAC |
| | | CGTGACCGTGTCCTCC |
| SEQ ID NO: 186 | HC | EVQLVQSGAEVKKPGESLRIS |
| | | CKGSGYTFTTYWMHWIRQSPSRGLE |
| | | WLGNIYPGTGGSNFDEKFKNRFTISR |
| | | DNSKNTLYLQMNSLRAEDTAVYYC |
| | | TRWTTGTGAYWGQGTTVTVSSAST |
| | | KGPSVFPLAPCSRSTSESTAALGCLV |
| | | KDYFPEPVTVSWNSGALTSGVHTFP |
| | | AVLQSSGLYSLSSVVTVPSSSLGTKT |
| | | YTCNVDHKPSNTKVDKRVESKYGPP |
| | | CPPCPAPEFLGGPSVFLFPPKPKDTL |
| | | MISRTPEVTCVVVDVSQEDPEVQFN |
| | | WYVDGVEVHNAKTKPREEQFNSTY |
| | | RVVSVLTVLHQDWLNGKEYKCKVS |
| | | NKGLPSSIEKTISKAKGQPREPQVYT |
| | | LPPSQEEMTKNQVSLTCLVKGFYPSD |
| | | IAVEWESNGQPENNYKTTPPVLDSD |
| | | GSFFLYSRLTVDKSRWQEGNVFSCS |
| | | VMHEALHNHYTQKSLSLSLGK |
| SEQ ID NO: 187 | DNA HC | GAAGTGCAGCTGGTGCAGTC |
| | | TGGAGCAGAGGTGAAAAAGCCCGG |
| | | GGAGTCTCTGAGGATCTCCTGTAA |
| | | GGGTTCTGGCTACACATTCACCACT |
| | | TACTGGATGCACTGGATCAGGCAG |
| | | TCCCCATCGAGAGGCCTTGAGTGG |
| | | CTGGGTAATATTTATCCTGGTACTG |
| | | GTGGTTCTAACTTCGATGAGAAGTT |
| | | CAAGAACAGATTCACCATCTCCAG |
| | | AGACAATTCCAAGAACACGCTGTA |
| | | TCTTCAAATGAACAGCCTGAGAGC |
| | | CGAGGACACGGCCGTGTATTACTG |
| | | TACAAGATGGACTACTGGGACGGG |
| | | AGCTTATTGGGGCCAGGGCACCAC |
| | | CGTGACCGTGTCCTCCGCTTCCACC |
| | | AAGGGCCCATCCGTCTTCCCCCTGG |
| | | CGCCCTGCTCCAGGAGCACCTCCG |
| | | AGAGCACAGCCGCCCTGGGCTGCC |
| | | TGGTCAAGGACTACTTCCCCGAAC |
| | | CGGTGACGGTGTCGTGGAACTCAG |
| | | GCGCCCTGACCAGCGGCGTGCACA |
| | | CCTTCCCGGCTGTCCTACAGTCCTC |
| | | AGGACTCTACTCCCTCAGCAGCGT |
| | | GGTGACCGTGCCCTCCAGCAGCTT |
| | | GGGCACGAAGACCTACACCTGCAA |
| | | CGTAGATCACAAGCCCAGCAACAC |
| | | CAAGGTGGACAAGAGAGTTGAGTC |
| | | CAAATATGGTCCCCCATGCCCACC |
| | | GTGCCCAGCACCTGAGTTCCTGGG |
| | | GGGACCATCAGTCTTCCTGTTCCCC |
| | | CCAAAACCCAAGGACACTCTCATG |
| | | ATCTCCCGGACCCCTGAGGTCACGT |
| | | GCGTGGTGGTGGACGTGAGCCAGG |
| | | AAGACCCCGAGGTCCAGTTCAACT |
| | | GGTACGTGGATGGCGTGGAGGTGC |
| | | ATAATGCCAAGACAAAGCCGCGGG |
| | | AGGAGCAGTTCAACAGCACGTACC |
| | | GTGTGGTCAGCGTCCTCACCGTCCT |
| | | GCACCAGGACTGGCTGAACGGCAA |
| | | GGAGTACAAGTGCAAGGTGTCCAA |
| | | CAAAGGCCTCCCGTCCTCCATCGA |
| | | GAAAACCATCTCCAAAGCCAAAGG |
| | | GCAGCCCCGAGAGCCACAGGTGTA |
| | | CACCCTGCCCCCATCCCAGGAGGA |
| | | GATGACCAAGAACCAGGTCAGCCT |
| | | GACCTGCCTGGTCAAAGGCTTCTAC |
| | | CCCAGCGACATCGCCGTGGAGTGG |
| | | GAGAGCAATGGGCAGCCGGAGAAC |
| | | AACTACAAGACCACGCCTCCCGTG |
| | | CTGGACTCCGACGGCTCCTTCTTCC |
| | | TCTACAGCAGGCTAACCGTGGACA |
| | | AGAGCAGGTGGCAGGAGGGGAAT |
| | | GTCTTCTCATGCTCCGTGATGCATG |
| | | AGGCTCTGCACAACCACTACACAC |
| | | AGAAGAGCCTCTCCCTGTCTCTGGG |
| | | TAAA |
| BAP049-hum04 LC | | |
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 | LCDR1 | SQSLLDSGNQKNF |
| (Chothia) | | |
| SEQ ID NO: 150 | LCDR2 | WAS |
| (Chothia) | | |
| SEQ ID NO: 167 | LCDR3 | DYSYPY |
| (Chothia) | | |
| SEQ ID NO: 188 | VL | EIVLTQSPATLSLSPGERATLS |
| | | CKSSQSLLDSGNQKNFLTWYQQKPG |
| | | KAPKLLIYWASTRESGVPSRFSGSGS |
| | | GTDFTFTISSLQPEDIATYYCQNDYS |
| | | YPYTFGQGTKVEIK |
| SEQ ID NO: 189 | DNA VL | GAAATTGTGTTGACACAGTC |
| | | TCCAGCCACCCTGTCTTTGTCTCCA |
| | | GGGGAAAGAGCCACCCTCTCCTGC |
| | | AAGTCCAGTCAGAGTCTGTTAGAC |
| | | AGTGGAAATCAAAAGAACTTCTTG |
| | | ACCTGGTATCAGCAGAAACCAGGG |
| | | AAAGCTCCTAAGCTCCTGATCTATT |
| | | GGGCATCCACTAGGGAATCTGGGG |
| | | TCCCATCAAGGTTCAGTGGAAGTG |
| | | GATCTGGGACAGATTTTACTTTCAC |
| | | CATCAGCAGCCTGCAGCCTGAAGA |
| | | TATTGCAACATATTACTGTCAGAAT |
| | | GATTATAGTTATCCGTACACGTTCG |
| | | GCCAAGGGACCAAGGTGGAAATCA |
| | | AA |
| SEQ ID NO: 190 | LC | EIVLTQSPATLSLSPGERATLS |
| | | CKSSQSLLDSGNQKNFLTWYQQKPG |
| | | KAPKLLIYWASTRESGVPSRFSGSGS |
| | | GTDFTFTISSLQPEDIATYYCQNDYS |
| | | YPYTFGQGTKVEIKRTVAAPSVFIFPP |
| | | SDEQLKSGTASVVCLLNNFYPREAK |
| | | VQWKVDNALQSGNSQESVTEQDSK |
| | | DSTYSLSSTLTLSKADYEKHKVYAC |
| | | EVTHQGLSSPVTKSFNRGEC |
| SEQ ID NO: 191 | DNA LC | GAAATTGTGTTGACACAGTC |
| | | TCCAGCCACCCTGTCTTTGTCTCCA |
| | | GGGGAAAGAGCCACCCTCTCCTGC |
| | | AAGTCCAGTCAGAGTCTGTTAGAC |
| | | AGTGGAAATCAAAAGAACTTCTTG |
| | | ACCTGGTATCAGCAGAAACCAGGG |
| | | AAAGCTCCTAAGCTCCTGATCTATT |
| | | GGGCATCCACTAGGGAATCTGGGG |
| | | TCCCATCAAGGTTCAGTGGAAGTG |
| | | GATCTGGGACAGATTTTACTTTCAC |
| | | CATCAGCAGCCTGCAGCCTGAAGA |
| | | TATTGCAACATATTACTGTCAGAAT |
| | | GATTATAGTTATCCGTACACGTTCG |
| | | GCCAAGGGACCAAGGTGGAAATCA |
| | | AACGTACGGTGGCTGCACCATCTG |
| | | TCTTCATCTTCCCGCCATCTGATGA |
| | | GCAGTTGAAATCTGGAACTGCCTCT |
| | | GTTGTGTGCCTGCTGAATAACTTCT |
| | | ATCCCAGAGAGGCCAAAGTACAGT |
| | | GGAAGGTGGATAACGCCCTCCAAT |
| | | CGGGTAACTCCCAGGAGAGTGTCA |
| | | CAGAGCAGGACAGCAAGGACAGC |
| | | ACCTACAGCCTCAGCAGCACCCTG |
| | | ACGCTGAGCAAAGCAGACTACGAG |
| | | AAACACAAAGTCTACGCCTGCGAA |
| | | GTCACCCATCAGGGCCTGAGCTCG |
| | | CCCGTCACAAAGAGCTTCAACAGG |
| | | GGAGAGTGT |
| BAP049-hum05 HC | | |
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 | HCDR1 | GYTFTTY |
| (Chothia) | | |
| SEQ ID NO: 141 | HCDR2 | YPGTGG |
| (Chothia) | | |
| SEQ ID NO: 139 | HCDR3 | WTTGTGAY |
| (Chothia) | | |
| SEQ ID NO: 172 | VH | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSS |
| SEQ ID NO: 173 | DNA VH | GAAGTGCAGCTGGTGCAGTC |
| | | TGGAGCAGAGGTGAAAAAGCCCGG |
| | | GGAGTCTCTGAGGATCTCCTGTAA |
| | | GGGTTCTGGCTACACATTCACCACT |
| | | TACTGGATGCACTGGGTGCGACAG |
| | | GCCACTGGACAAGGGCTTGAGTGG |
| | | ATGGGTAATATTTATCCTGGTACTG |
| | | GTGGTTCTAACTTCGATGAGAAGTT |
| | | CAAGAACAGAGTCACGATTACCGC |
| | | GGACAAATCCACGAGCACAGCCTA |
| | | CATGGAGCTGAGCAGCCTGAGATC |
| | | TGAGGACACGGCCGTGTATTACTG |
| | | TACAAGATGGACTACTGGGACGGG |
| | | AGCTTATTGGGGCCAGGGCACCAC |
| | | CGTGACCGTGTCCTCC |
| SEQ ID NO: 174 | HC | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSSAS |
| | | TKGPSVFPLAPCSRSTSESTAALGCL |
| | | VKDYFPEPVTVSWNSGALTSGVHTF |
| | | PAVLQSSGLYSLSSVVTVPSSSLGTK |
| | | TYTCNVDHKPSNTKVDKRVESKYGP |
| | | PCPPCPAPEFLGGPSVFLFPPKPKDTL |
| | | MISRTPEVTCVVVDVSQEDPEVQFN |
| | | WYVDGVEVHNAKTKPREEQFNSTY |
| | | RVVSVLTVLHQDWLNGKEYKCKVS |
| | | NKGLPSSIEKTISKAKGQPREPQVYT |
| | | LPPSQEEMTKNQVSLTCLVKGFYPSD |
| | | IAVEWESNGQPENNYKTTPPVLDSD |
| | | GSFFLYSRLTVDKSRWQEGNVFSCS |
| | | VMHEALHNHYTQKSLSLSLGK |
| SEQ ID NO: 175 | DNA HC | GAAGTGCAGCTGGTGCAGTC |
| | | TGGAGCAGAGGTGAAAAAGCCCGG |
| | | GGAGTCTCTGAGGATCTCCTGTAA |
| | | GGGTTCTGGCTACACATTCACCACT |
| | | TACTGGATGCACTGGGTGCGACAG |
| | | GCCACTGGACAAGGGCTTGAGTGG |
| | | ATGGGTAATATTTATCCTGGTACTG |
| | | GTGGTTCTAACTTCGATGAGAAGTT |
| | | CAAGAACAGAGTCACGATTACCGC |
| | | GGACAAATCCACGAGCACAGCCTA |
| | | CATGGAGCTGAGCAGCCTGAGATC |
| | | TGAGGACACGGCCGTGTATTACTG |
| | | TACAAGATGGACTACTGGGACGGG |
| | | AGCTTATTGGGGCCAGGGCACCAC |
| | | CGTGACCGTGTCCTCCGCTTCCACC |
| | | AAGGGCCCATCCGTCTTCCCCCTGG |
| | | CGCCCTGCTCCAGGAGCACCTCCG |
| | | AGAGCACAGCCGCCCTGGGCTGCC |
| | | TGGTCAAGGACTACTTCCCCGAAC |
| | | CGGTGACGGTGTCGTGGAACTCAG |
| | | GCGCCCTGACCAGCGGCGTGCACA |
| | | CCTTCCCGGCTGTCCTACAGTCCTC |
| | | AGGACTCTACTCCCTCAGCAGCGT |
| | | GGTGACCGTGCCCTCCAGCAGCTT |
| | | GGGCACGAAGACCTACACCTGCAA |
| | | CGTAGATCACAAGCCCAGCAACAC |
| | | CAAGGTGGACAAGAGAGTTGAGTC |
| | | CAAATATGGTCCCCCATGCCCACC |
| | | GTGCCCAGCACCTGAGTTCCTGGG |
| | | GGGACCATCAGTCTTCCTGTTCCCC |
| | | CCAAAACCCAAGGACACTCTCATG |
| | | ATCTCCCGGACCCCTGAGGTCACGT |
| | | GCGTGGTGGTGGACGTGAGCCAGG |
| | | AAGACCCCGAGGTCCAGTTCAACT |
| | | GGTACGTGGATGGCGTGGAGGTGC |
| | | ATAATGCCAAGACAAAGCCGCGGG |
| | | AGGAGCAGTTCAACAGCACGTACC |
| | | GTGTGGTCAGCGTCCTCACCGTCCT |
| | | GCACCAGGACTGGCTGAACGGCAA |
| | | GGAGTACAAGTGCAAGGTGTCCAA |
| | | CAAAGGCCTCCCGTCCTCCATCGA |
| | | GAAAACCATCTCCAAAGCCAAAGG |
| | | GCAGCCCCGAGAGCCACAGGTGTA |
| | | CACCCTGCCCCCATCCCAGGAGGA |
| | | GATGACCAAGAACCAGGTCAGCCT |
| | | GACCTGCCTGGTCAAAGGCTTCTAC |
| | | CCCAGCGACATCGCCGTGGAGTGG |
| | | GAGAGCAATGGGCAGCCGGAGAAC |
| | | AACTACAAGACCACGCCTCCCGTG |
| | | CTGGACTCCGACGGCTCCTTCTTCC |
| | | TCTACAGCAGGCTAACCGTGGACA |
| | | AGAGCAGGTGGCAGGAGGGGAAT |
| | | GTCTTCTCATGCTCCGTGATGCATG |
| | | AGGCTCTGCACAACCACTACACAC |
| | | AGAAGAGCCTCTCCCTGTCTCTGGG |
| | | TAAA |
| BAP049-hum05 LC | | |
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 | LCDR1 | SQSLLDSGNQKNF |
| (Chothia) | | |
| SEQ ID NO: 150 | LCDR2 | WAS |
| (Chothia) | | |
| SEQ ID NO: 167 | LCDR3 | DYSYPY |
| (Chothia) | | |
| SEQ ID NO: 188 | VL | EIVLTQSPATLSLSPGERATLS |
| | | CKSSQSLLDSGNQKNFLTWYQQKPG |
| | | KAPKLLIYWASTRESGVPSRFSGSGS |
| | | GTDFTFTISSLQPEDIATYYCQNDYS |
| | | YPYTFGQGTKVEIK |
| SEQ ID NO: 189 | DNA VL | AAGAAATTGTGTTGACACAGTC |
| | | TCCAGCCACCCTGTCTTTGTCTCCA |
| | | GGGGAAAGAGCCACCCTCTCCTGC |
| | | AAGTCCAGTCAGAGTCTGTTAGAC |
| | | AGTGGAAATCAAAAGAACTTCTTG |
| | | ACCTGGTATCAGCAGAAACCAGGG |
| | | AAAGCTCCTAAGCTCCTGATCTATT |
| | | GGGCATCCACTAGGGAATCTGGGG |
| | | TCCCATCAAGGTTCAGTGGAAGTG |
| | | GATCTGGGACAGATTTTACTTTCAC |
| | | CATCAGCAGCCTGCAGCCTGAAGA |
| | | TATTGCAACATATTACTGTCAGAAT |
| | | GATTATAGTTATCCGTACACGTTCG |
| | | GCCAAGGGACCAAGGTGGAAATCA |
| SEQ ID NO: 190 | LC | EIVLTQSPATLSLSPGERATLS |
| | | CKSSQSLLDSGNQKNFLTWYQQKPG |
| | | KAPKLLIYWASTRESGVPSRFSGSGS |
| | | GTDFTFTISSLQPEDIATYYCQNDYS |
| | | YPYTFGQGTKVEIKRTVAAPSVFIFPP |
| | | SDEQLKSGTASVVCLLNNFYPREAK |
| | | VQWKVDNALQSGNSQESVTEQDSK |
| | | DSTYSLSSTLTLSKADYEKHKVYAC |
| | | EVTHQGLSSPVTKSFNRGEC |
| SEQ ID NO: 191 | DNA LC | GAAATTGTGTTGACACAGTC |
| | | TCCAGCCACCCTGTCTTTGTCTCCA |
| | | GGGGAAAGAGCCACCCTCTCCTGC |
| | | AAGTCCAGTCAGAGTCTGTTAGAC |
| | | AGTGGAAATCAAAAGAACTTCTTG |
| | | ACCTGGTATCAGCAGAAACCAGGG |
| | | AAAGCTCCTAAGCTCCTGATCTATT |
| | | GGGCATCCACTAGGGAATCTGGGG |
| | | TCCCATCAAGGTTCAGTGGAAGTG |
| | | GATCTGGGACAGATTTTACTTTCAC |
| | | CATCAGCAGCCTGCAGCCTGAAGA |
| | | TATTGCAACATATTACTGTCAGAAT |
| | | GATTATAGTTATCCGTACACGTTCG |
| | | GCCAAGGGACCAAGGTGGAAATCA |
| | | AACGTACGGTGGCTGCACCATCTG |
| | | TCTTCATCTTCCCGCCATCTGATGA |
| | | GCAGTTGAAATCTGGAACTGCCTCT |
| | | GTTGTGTGCCTGCTGAATAACTTCT |
| | | ATCCCAGAGAGGCCAAAGTACAGT |
| | | GGAAGGTGGATAACGCCCTCCAAT |
| | | CGGGTAACTCCCAGGAGAGTGTCA |
| | | CAGAGCAGGACAGCAAGGACAGC |
| | | ACCTACAGCCTCAGCAGCACCCTG |
| | | ACGCTGAGCAAAGCAGACTACGAG |
| | | AAACACAAAGTCTACGCCTGCGAA |
| | | GTCACCCATCAGGGCCTGAGCTCG |
| | | CCCGTCACAAAGAGCTTCAACAGG |
| | | GGAGAGTGT |
| BAP049-hum06 HC | | |
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 | HCDR1 | GYTFTTY |
| (Chothia) | | |
| SEQ ID NO: 141 | HCDR2 | YPGTGG |
| (Chothia) | | |
| SEQ ID NO: 139 | HCDR3 | WTTGTGAY |
| (Chothia) | | |
| SEQ ID NO: 172 | VH | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSS |
| SEQ ID NO: 173 | DNA VH | GAAGTGCAGCTGGTGCAGTC |
| | | TGGAGCAGAGGTGAAAAAGCCCGG |
| | | GGAGTCTCTGAGGATCTCCTGTAA |
| | | GGGTTCTGGCTACACATTCACCACT |
| | | TACTGGATGCACTGGGTGCGACAG |
| | | GCCACTGGACAAGGGCTTGAGTGG |
| | | ATGGGTAATATTTATCCTGGTACTG |
| | | GTGGTTCTAACTTCGATGAGAAGTT |
| | | CAAGAACAGAGTCACGATTACCGC |
| | | GGACAAATCCACGAGCACAGCCTA |
| | | CATGGAGCTGAGCAGCCTGAGATC |
| | | TGAGGACACGGCCGTGTATTACTG |
| | | TACAAGATGGACTACTGGGACGGG |
| | | AGCTTATTGGGGCCAGGGCACCAC |
| | | CGTGACCGTGTCCTCC |
| SEQ ID NO: 174 | HC | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSSAS |
| | | TKGPSVFPLAPCSRSTSESTAALGCL |
| | | VKDYFPEPVTVSWNSGALTSGVHTF |
| | | PAVLQSSGLYSLSSVVTVPSSSLGTK |
| | | TYTCNVDHKPSNTKVDKRVESKYGP |
| | | PCPPCPAPEFLGGPSVFLFPPKPKDTL |
| | | MISRTPEVTCVVVDVSQEDPEVQFN |
| | | WYVDGVEVHNAKTKPREEQFNSTY |
| | | RVVSVLTVLHQDWLNGKEYKCKVS |
| | | NKGLPSSIEKTISKAKGQPREPQVYT |
| | | LPPSQEEMTKNQVSLTCLVKGFYPSD |
| | | IAVEWESNGQPENNYKTTPPVLDSD |
| | | GSFFLYSRLTVDKSRWQEGNVFSCS |
| | | VMHEALHNHYTQKSLSLSLGK |
| SEQ ID NO: 175 | DNA HC | GAAGTGCAGCTGGTGCAGTC |
| | | TGGAGCAGAGGTGAAAAAGCCCGG |
| | | GGAGTCTCTGAGGATCTCCTGTAA |
| | | GGGTTCTGGCTACACATTCACCACT |
| | | TACTGGATGCACTGGGTGCGACAG |
| | | GCCACTGGACAAGGGCTTGAGTGG |
| | | ATGGGTAATATTTATCCTGGTACTG |
| | | GTGGTTCTAACTTCGATGAGAAGTT |
| | | CAAGAACAGAGTCACGATTACCGC |
| | | GGACAAATCCACGAGCACAGCCTA |
| | | CATGGAGCTGAGCAGCCTGAGATC |
| | | TGAGGACACGGCCGTGTATTACTG |
| | | TACAAGATGGACTACTGGGACGGG |
| | | AGCTTATTGGGGCCAGGGCACCAC |
| | | CGTGACCGTGTCCTCCGCTTCCACC |
| | | AAGGGCCCATCCGTCTTCCCCCTGG |
| | | CGCCCTGCTCCAGGAGCACCTCCG |
| | | AGAGCACAGCCGCCCTGGGCTGCC |
| | | TGGTCAAGGACTACTTCCCCGAAC |
| | | CGGTGACGGTGTCGTGGAACTCAG |
| | | GCGCCCTGACCAGCGGCGTGCACA |
| | | CCTTCCCGGCTGTCCTACAGTCCTC |
| | | AGGACTCTACTCCCTCAGCAGCGT |
| | | GGTGACCGTGCCCTCCAGCAGCTT |
| | | GGGCACGAAGACCTACACCTGCAA |
| | | CGTAGATCACAAGCCCAGCAACAC |
| | | CAAGGTGGACAAGAGAGTTGAGTC |
| | | CAAATATGGTCCCCCATGCCCACC |
| | | GTGCCCAGCACCTGAGTTCCTGGG |
| | | GGGACCATCAGTCTTCCTGTTCCCC |
| | | CCAAAACCCAAGGACACTCTCATG |
| | | ATCTCCCGGACCCCTGAGGTCACGT |
| | | GCGTGGTGGTGGACGTGAGCCAGG |
| | | AAGACCCCGAGGTCCAGTTCAACT |
| | | GGTACGTGGATGGCGTGGAGGTGC |
| | | ATAATGCCAAGACAAAGCCGCGGG |
| | | AGGAGCAGTTCAACAGCACGTACC |
| | | GTGTGGTCAGCGTCCTCACCGTCCT |
| | | GCACCAGGACTGGCTGAACGGCAA |
| | | GGAGTACAAGTGCAAGGTGTCCAA |
| | | CAAAGGCCTCCCGTCCTCCATCGA |
| | | GAAAACCATCTCCAAAGCCAAAGG |
| | | GCAGCCCCGAGAGCCACAGGTGTA |
| | | CACCCTGCCCCCATCCCAGGAGGA |
| | | GATGACCAAGAACCAGGTCAGCCT |
| | | GACCTGCCTGGTCAAAGGCTTCTAC |
| | | CCCAGCGACATCGCCGTGGAGTGG |
| | | GAGAGCAATGGGCAGCCGGAGAAC |
| | | AACTACAAGACCACGCCTCCCGTG |
| | | CTGGACTCCGACGGCTCCTTCTTCC |
| | | TCTACAGCAGGCTAACCGTGGACA |
| | | AGAGCAGGTGGCAGGAGGGGAAT |
| | | GTCTTCTCATGCTCCGTGATGCATG |
| | | AGGCTCTGCACAACCACTACACAC |
| | | AGAAGAGCCTCTCCCTGTCTCTGGG |
| | | TAAA |
| BAP049-hum06 LC | | |
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 | LCDR1 | SQSLLDSGNQKNF |
| (Chothia) | | |
| SEQ ID NO: 150 | LCDR2 | WAS |
| (Chothia) | | |
| SEQ ID NO: 167 | LCDR3 | DYSYPY |
| (Chothia) | | |
| SEQ ID NO: 192 | VL | DIVMTQTPLSLPVTPGEPASIS |
| | | CKSSQSLLDSGNQKNFLTWYQQKPG |
| | | QAPRLLIYWASTRESGVPSRFSGSGS |
| | | GTDFTFTISSLEAEDAATYYCQNDYS |
| | | YPYTFGQGTKVEIK |
| SEQ ID NO: 193 | DNA VL | GATATTGTGATGACCCAGAC |
| | | TCCACTCTCCCTGCCCGTCACCCCT |
| | | GGAGAGCCGGCCTCCATCTCCTGC |
| | | AAGTCCAGTCAGAGTCTGTTAGAC |
| | | AGTGGAAATCAAAAGAACTTCTTG |
| | | ACCTGGTACCAGCAGAAACCTGGC |
| | | CAGGCTCCCAGGCTCCTCATCTATT |
| | | GGGCATCCACTAGGGAATCTGGGG |
| | | TCCCCTCGAGGTTCAGTGGCAGTG |
| | | GATCTGGGACAGATTTCACCTTTAC |
| | | CATCAGTAGCCTGGAAGCTGAAGA |
| | | TGCTGCAACATATTACTGTCAGAAT |
| | | GATTATAGTTATCCGTACACGTTCG |
| | | GCCAAGGGACCAAGGTGGAAATCA |
| | | AA |
| SEQ ID NO: 194 | LC | DIVMTQTPLSLPVTPGEPASIS |
| | | CKSSQSLLDSGNQKNFLTWYQQKPG |
| | | QAPRLLIYWASTRESGVPSRFSGSGS |
| | | GTDFTFTISSLEAEDAATYYCQNDYS |
| | | YPYTFGQGTKVEIKRTVAAPSVFIFPP |
| | | SDEQLKSGTASVVCLLNNFYPREAK |
| | | VQWKVDNALQSGNSQESVTEQDSK |
| | | DSTYSLSSTLTLSKADYEKHKVYAC |
| | | EVTHQGLSSPVTKSFNRGEC |
| SEQ ID NO: 195 | DNA LC | GATATTGTGATGACCCAGAC |
| | | TCCACTCTCCCTGCCCGTCACCCCT |
| | | GGAGAGCCGGCCTCCATCTCCTGC |
| | | AAGTCCAGTCAGAGTCTGTTAGAC |
| | | AGTGGAAATCAAAAGAACTTCTTG |
| | | ACCTGGTACCAGCAGAAACCTGGC |
| | | CAGGCTCCCAGGCTCCTCATCTATT |
| | | GGGCATCCACTAGGGAATCTGGGG |
| | | TCCCCTCGAGGTTCAGTGGCAGTG |
| | | GATCTGGGACAGATTTCACCTTTAC |
| | | CATCAGTAGCCTGGAAGCTGAAGA |
| | | TGCTGCAACATATTACTGTCAGAAT |
| | | GATTATAGTTATCCGTACACGTTCG |
| | | GCCAAGGGACCAAGGTGGAAATCA |
| | | AACGTACGGTGGCTGCACCATCTG |
| | | TCTTCATCTTCCCGCCATCTGATGA |
| | | GCAGTTGAAATCTGGAACTGCCTCT |
| | | GTTGTGTGCCTGCTGAATAACTTCT |
| | | ATCCCAGAGAGGCCAAAGTACAGT |
| | | GGAAGGTGGATAACGCCCTCCAAT |
| | | CGGGTAACTCCCAGGAGAGTGTCA |
| | | CAGAGCAGGACAGCAAGGACAGC |
| | | ACCTACAGCCTCAGCAGCACCCTG |
| | | ACGCTGAGCAAAGCAGACTACGAG |
| | | AAACACAAAGTCTACGCCTGCGAA |
| | | GTCACCCATCAGGGCCTGAGCTCG |
| | | CCCGTCACAAAGAGCTTCAACAGG |
| | | GGAGAGTGT |
| BAP049-hum07 HC | | |
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 | HCDR1 | GYTFTTY |
| (Chothia) | | |
| SEQ ID NO: 141 | HCDR2 | YPGTGG |
| (Chothia) | | |
| SEQ ID NO: 139 | HCDR3 | WTTGTGAY |
| (Chothia) | | |
| SEQ ID NO: 172 | VH | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSS |
| SEQ ID NO: 173 | DNA VH | GAAGTGCAGCTGGTGCAGTC |
| | | TGGAGCAGAGGTGAAAAAGCCCGG |
| | | GGAGTCTCTGAGGATCTCCTGTAA |
| | | GGGTTCTGGCTACACATTCACCACT |
| | | TACTGGATGCACTGGGTGCGACAG |
| | | GCCACTGGACAAGGGCTTGAGTGG |
| | | ATGGGTAATATTTATCCTGGTACTG |
| | | GTGGTTCTAACTTCGATGAGAAGTT |
| | | CAAGAACAGAGTCACGATTACCGC |
| | | GGACAAATCCACGAGCACAGCCTA |
| | | CATGGAGCTGAGCAGCCTGAGATC |
| | | TGAGGACACGGCCGTGTATTACTG |
| | | TACAAGATGGACTACTGGGACGGG |
| | | AGCTTATTGGGGCCAGGGCACCAC |
| | | CGTGACCGTGTCCTCC |
| SEQ ID NO: 174 | HC | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSAS |
| | | TKGPSVFPLAPCSRSTSESTAALGCL |
| | | VKDYFPEPVTVSWNSGALTSGVHTF |
| | | PAVLQSSGLYSLSSVVTVPSSSLGTK |
| | | TYTCNVDHKPSNTKVDKRVESKYGP |
| | | PCPPCPAPEFLGGPSVFLFPPKPKDTL |
| | | MISRTPEVTCVVVDVSQEDPEVQFN |
| | | WYVDGVEVHNAKTKPREEQFNSTY |
| | | RVVSVLTVLHQDWLNGKEYKCKVS |
| | | NKGLPSSIEKTISKAKGQPREPQVYT |
| | | LPPSQEEMTKNQVSLTCLVKGFYPSD |
| | | IAVEWESNGQPENNYKTTPPVLDSD |
| | | GSFFLYSRLTVDKSRWQEGNVFSCS |
| | | VMHEALHNHYTQKSLSLSLGK |
| SEQ ID NO: 175 | DNA HC | GAAGTGCAGCTGGTGCAGTC |
| | | TGGAGCAGAGGTGAAAAAGCCCGG |
| | | GGAGTCTCTGAGGATCTCCTGTAA |
| | | GGGTTCTGGCTACACATTCACCACT |
| | | TACTGGATGCACTGGGTGCGACAG |
| | | GCCACTGGACAAGGGCTTGAGTGG |
| | | ATGGGTAATATTTATCCTGGTACTG |
| | | GTGGTTCTAACTTCGATGAGAAGTT |
| | | CAAGAACAGAGTCACGATTACCGC |
| | | GGACAAATCCACGAGCACAGCCTA |
| | | CATGGAGCTGAGCAGCCTGAGATC |
| | | TGAGGACACGGCCGTGTATTACTG |
| | | TACAAGATGGACTACTGGGACGGG |
| | | AGCTTATTGGGGCCAGGGCACCAC |
| | | CGTGACCGTGTCCTCCGCTTCCACC |
| | | AAGGGCCCATCCGTCTTCCCCCTGG |
| | | CGCCCTGCTCCAGGAGCACCTCCG |
| | | AGAGCACAGCCGCCCTGGGCTGCC |
| | | TGGTCAAGGACTACTTCCCCGAAC |
| | | CGGTGACGGTGTCGTGGAACTCAG |
| | | GCGCCCTGACCAGCGGCGTGCACA |
| | | CCTTCCCGGCTGTCCTACAGTCCTC |
| | | AGGACTCTACTCCCTCAGCAGCGT |
| | | GGTGACCGTGCCCTCCAGCAGCTT |
| | | GGGCACGAAGACCTACACCTGCAA |
| | | CGTAGATCACAAGCCCAGCAACAC |
| | | CAAGGTGGACAAGAGAGTTGAGTC |
| | | CAAATATGGTCCCCCATGCCCACC |
| | | GTGCCCAGCACCTGAGTTCCTGGG |
| | | GGGACCATCAGTCTTCCTGTTCCCC |
| | | CCAAAACCCAAGGACACTCTCATG |
| | | ATCTCCCGGACCCCTGAGGTCACGT |
| | | GCGTGGTGGTGGACGTGAGCCAGG |
| | | AAGACCCCGAGGTCCAGTTCAACT |
| | | GGTACGTGGATGGCGTGGAGGTGC |
| | | ATAATGCCAAGACAAAGCCGCGGG |
| | | AGGAGCAGTTCAACAGCACGTACC |
| | | GTGTGGTCAGCGTCCTCACCGTCCT |
| | | GCACCAGGACTGGCTGAACGGCAA |
| | | GGAGTACAAGTGCAAGGTGTCCAA |
| | | CAAAGGCCTCCCGTCCTCCATCGA |
| | | GAAAACCATCTCCAAAGCCAAAGG |
| | | GCAGCCCCGAGAGCCACAGGTGTA |
| | | CACCCTGCCCCCATCCCAGGAGGA |
| | | GATGACCAAGAACCAGGTCAGCCT |
| | | GACCTGCCTGGTCAAAGGCTTCTAC |
| | | CCCAGCGACATCGCCGTGGAGTGG |
| | | GAGAGCAATGGGCAGCCGGAGAAC |
| | | AACTACAAGACCACGCCTCCCGTG |
| | | CTGGACTCCGACGGCTCCTTCTTCC |
| | | TCTACAGCAGGCTAACCGTGGACA |
| | | AGAGCAGGTGGCAGGAGGGGAAT |
| | | GTCTTCTCATGCTCCGTGATGCATG |
| | | AGGCTCTGCACAACCACTACACAC |
| | | AGAAGAGCCTCTCCCTGTCTCTGGG |
| | | TAAA |
| BAP049-hum07 LC | | |
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 | LCDR1 | SQSLLDSGNQKNF |
| (Chothia) | | |
| SEQ ID NO: 150 | LCDR2 | WAS |
| (Chothia) | | |
| SEQ ID NO: 167 | LCDR3 | DYSYPY |
| (Chothia) | | |
| SEQ ID NO: 196 | VL | EIVLTQSPATLSLSPGERATLS |
| | | CKSSQSLLDSGNQKNFLTWYQQKPG |
| | | KAPKLLIYWASTRESGVPSRFSGSGS |
| | | GTDFTFTISSLEAEDAATYYCQNDYS |
| | | PYTFGQGTKVEIK |
| SEQ ID NO: 197 | DNA VL | GAAATTGTGTTGACACAGTC |
| | | TCCAGCCACCCTGTCTTTGTCTCCA |
| | | GGGGAAAGAGCCACCCTCTCCTGC |
| | | AAGTCCAGTCAGAGTCTGTTAGAC |
| | | AGTGGAAATCAAAAGAACTTCTTG |
| | | ACCTGGTATCAGCAGAAACCAGGG |
| | | AAAGCTCCTAAGCTCCTGATCTATT |
| | | GGGCATCCACTAGGGAATCTGGGG |
| | | TCCCCTCGAGGTTCAGTGGCAGTG |
| | | GATCTGGGACAGATTTCACCTTTAC |
| | | CATCAGTAGCCTGGAAGCTGAAGA |
| | | TGCTGCAACATATTACTGTCAGAAT |
| | | GATTATAGTTATCCGTACACGTTCG |
| | | GCCAAGGGACCAAGGTGGAAATCA |
| | | AA |
| SEQ ID NO: 198 | LC | EIVLTQSPATLSLSPGERATLS |
| | | CKSSQSLLDSGNQKNFLTWYQQKPG |
| | | KAPKLLIYWASTRESGVPSRFSGSGS |
| | | GTDFTFTISSLEAEDAATYYCQNDYS |
| | | YPYTFGQGTKVEIKRTVAAPSVFIFPP |
| | | SDEQLKSGTASVVCLLNNFYPREAK |
| | | VQWKVDNALQSGNSQESVTEQDSK |
| | | DSTYSLSSTLTLSKADYEKHKVYAC |
| | | EVTHQGLSSPVTKSFNRGEC |
| SEQ ID NO: 199 | DNA LC | GAAATTGTGTTGACACAGTC |
| | | TCCAGCCACCCTGTCTTTGTCTCCA |
| | | GGGGAAAGAGCCACCCTCTCCTGC |
| | | AAGTCCAGTCAGAGTCTGTTAGAC |
| | | AGTGGAAATCAAAAGAACTTCTTG |
| | | ACCTGGTATCAGCAGAAACCAGGG |
| | | AAAGCTCCTAAGCTCCTGATCTATT |
| | | GGGCATCCACTAGGGAATCTGGGG |
| | | TCCCCTCGAGGTTCAGTGGCAGTG |
| | | GATCTGGGACAGATTTCACCTTTAC |
| | | CATCAGTAGCCTGGAAGCTGAAGA |
| | | TGCTGCAACATATTACTGTCAGAAT |
| | | GATTATAGTTATCCGTACACGTTCG |
| | | GCCAAGGGACCAAGGTGGAAATCA |
| | | AACGTACGGTGGCTGCACCATCTG |
| | | TCTTCATCTTCCCGCCATCTGATGA |
| | | GCAGTTGAAATCTGGAACTGCCTCT |
| | | GTTGTGTGCCTGCTGAATAACTTCT |
| | | ATCCCAGAGAGGCCAAAGTACAGT |
| | | GGAAGGTGGATAACGCCCTCCAAT |
| | | CGGGTAACTCCCAGGAGAGTGTCA |
| | | CAGAGCAGGACAGCAAGGACAGC |
| | | ACCTACAGCCTCAGCAGCACCCTG |
| | | ACGCTGAGCAAAGCAGACTACGAG |
| | | AAACACAAAGTCTACGCCTGCGAA |
| | | GTCACCCATCAGGGCCTGAGCTCG |
| | | CCCGTCACAAAGAGCTTCAACAGG |
| | | GGAGAGTGT |
| BAP049-hum08 HC | | |
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 | HCDR1 | GYTFTTY |
| (Chothia) | | |
| SEQ ID NO: 141 | HCDR2 | YPGTGG |
| (Chothia) | | |
| SEQ ID NO: 139 | HCDR3 | WTTGTGAY |
| (Chothia) | | |
| SEQ ID NO: 184 | VH | EVQLVQSGAEVKKPGESLRIS |
| | | CKGSGYTFTTYWMHWIRQSPSRGLE |
| | | WLGNIYPGTGGSNFDEKFKNRFTISR |
| | | DNSKNTLYLQMNSLRAEDTAVYYC |
| | | TRWTTGTGAYWGQGTTVTVSS |
| SEQ ID NO: 185 | DNA VH | GAAGTGCAGCTGGTGCAGTC |
| | | TGGAGCAGAGGTGAAAAAGCCCGG |
| | | GGAGTCTCTGAGGATCTCCTGTAA |
| | | GGGTTCTGGCTACACATTCACCACT |
| | | TACTGGATGCACTGGATCAGGCAG |
| | | TCCCCATCGAGAGGCCTTGAGTGG |
| | | CTGGGTAATATTTATCCTGGTACTG |
| | | GTGGTTCTAACTTCGATGAGAAGTT |
| | | CAAGAACAGATTCACCATCTCCAG |
| | | AGACAATTCCAAGAACACGCTGTA |
| | | TCTTCAAATGAACAGCCTGAGAGC |
| | | CGAGGACACGGCCGTGTATTACTG |
| | | TACAAGATGGACTACTGGGACGGG |
| | | AGCTTATTGGGGCCAGGGCACCAC |
| | | CGTGACCGTGTCCTCC |
| SEQ ID NO: 186 | HC | EVQLVQSGAEVKKPGESLRIS |
| | | CKGSGYTFTTYWMHWIRQSPSRGLE |
| | | WLGNIYPGTGGSNFDEKFKNRFTISR |
| | | DNSKNTLYLQMNSLRAEDTAVYYC |
| | | TRWTTGTGAYWGQGTTVTVSSAST |
| | | KGPSVFPLAPCSRSTSESTAALGCLV |
| | | KDYFPEPVTVSWNSGALTSGVHTFP |
| | | AVLQSSGLYSLSSVVTVPSSSLGTKT |
| | | YTCNVDHKPSNTKVDKRVESKYGPP |
| | | CPPCPAPEFLGGPSVFLFPPKPKDTL |
| | | MISRTPEVTCVVVDVSQEDPEVQFN |
| | | WYVDGVEVHNAKTKPREEQFNSTY |
| | | RVVSVLTVLHQDWLNGKEYKCKVS |
| | | NKGLPSSIEKTISKAKGQPREPQVYT |
| | | LPPSQEEMTKNQVSLTCLVKGFYPSD |
| | | IAVEWESNGQPENNYKTTPPVLDSD |
| | | GSFFLYSRLTVDKSRWQEGNVFSCS |
| | | VMHEALHNHYTQKSLSLSLGK |
| SEQ ID NO: 187 | DNA HC | GAAGTGCAGCTGGTGCAGTC |
| | | TGGAGCAGAGGTGAAAAAGCCCGG |
| | | GGAGTCTCTGAGGATCTCCTGTAA |
| | | GGGTTCTGGCTACACATTCACCACT |
| | | TACTGGATGCACTGGATCAGGCAG |
| | | TCCCCATCGAGAGGCCTTGAGTGG |
| | | CTGGGTAATATTTATCCTGGTACTG |
| | | GTGGTTCTAACTTCGATGAGAAGTT |
| | | CAAGAACAGATTCACCATCTCCAG |
| | | AGACAATTCCAAGAACACGCTGTA |
| | | TCTTCAAATGAACAGCCTGAGAGC |
| | | CGAGGACACGGCCGTGTATTACTG |
| | | TACAAGATGGACTACTGGGACGGG |
| | | AGCTTATTGGGGCCAGGGCACCAC |
| | | CGTGACCGTGTCCTCCGCTTCCACC |
| | | AAGGGCCCATCCGTCTTCCCCCTGG |
| | | CGCCCTGCTCCAGGAGCACCTCCG |
| | | AGAGCACAGCCGCCCTGGGCTGCC |
| | | TGGTCAAGGACTACTTCCCCGAAC |
| | | CGGTGACGGTGTCGTGGAACTCAG |
| | | GCGCCCTGACCAGCGGCGTGCACA |
| | | CCTTCCCGGCTGTCCTACAGTCCTC |
| | | AGGACTCTACTCCCTCAGCAGCGT |
| | | GGTGACCGTGCCCTCCAGCAGCTT |
| | | GGGCACGAAGACCTACACCTGCAA |
| | | CGTAGATCACAAGCCCAGCAACAC |
| | | CAAGGTGGACAAGAGAGTTGAGTC |
| | | CAAATATGGTCCCCCATGCCCACC |
| | | GTGCCCAGCACCTGAGTTCCTGGG |
| | | GGGACCATCAGTCTTCCTGTTCCCC |
| | | CCAAAACCCAAGGACACTCTCATG |
| | | ATCTCCCGGACCCCTGAGGTCACGT |
| | | GCGTGGTGGTGGACGTGAGCCAGG |
| | | AAGACCCCGAGGTCCAGTTCAACT |
| | | GGTACGTGGATGGCGTGGAGGTGC |
| | | ATAATGCCAAGACAAAGCCGCGGG |
| | | AGGAGCAGTTCAACAGCACGTACC |
| | | GTGTGGTCAGCGTCCTCACCGTCCT |
| | | GCACCAGGACTGGCTGAACGGCAA |
| | | GGAGTACAAGTGCAAGGTGTCCAA |
| | | CAAAGGCCTCCCGTCCTCCATCGA |
| | | GAAAACCATCTCCAAAGCCAAAGG |
| | | GCAGCCCCGAGAGCCACAGGTGTA |
| | | CACCCTGCCCCCATCCCAGGAGGA |
| | | GATGACCAAGAACCAGGTCAGCCT |
| | | GACCTGCCTGGTCAAAGGCTTCTAC |
| | | CCCAGCGACATCGCCGTGGAGTGG |
| | | GAGAGCAATGGGCAGCCGGAGAAC |
| | | AACTACAAGACCACGCCTCCCGTG |
| | | CTGGACTCCGACGGCTCCTTCTTCC |
| | | TCTACAGCAGGCTAACCGTGGACA |
| | | AGAGCAGGTGGCAGGAGGGGAAT |
| | | GTCTTCTCATGCTCCGTGATGCATG |
| | | AGGCTCTGCACAACCACTACACAC |
| | | AGAAGAGCCTCTCCCTGTCTCTGGG |
| | | TAAA |
| BAP049-hum08 LC | | |
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 | LCDR1 | SQSLLDSGNQKNF |
| (Chothia) | | |
| SEQ ID NO: 150 | LCDR2 | WAS |
| (Chothia) | | |
| SEQ ID NO: 167 | LCDR3 | DYSYPY |
| (Chothia) | | |
| SEQ ID NO: 200 | VL | EIVLTQSPDFQSVTPKEKVTIT |
| | | CKSSQSLLDSGNQKNFLTWYQQKPG |
| | | QAPRLLIYWASTRESGVPSRFSGSGS |
| | | GTDFTFTISSLEAEDAATYYCQNDYS |
| | | YPYTFGQGTKVEIK |
| SEQ ID NO: 201 | DNA VL | GAAATTGTGCTGACTCAGTC |
| | | TCCAGACTTTCAGTCTGTGACTCCA |
| | | AAGGAGAAAGTCACCATCACCTGC |
| | | AAGTCCAGTCAGAGTCTGTTAGAC |
| | | AGTGGAAATCAAAAGAACTTCTTG |
| | | ACCTGGTACCAGCAGAAACCTGGC |
| | | CAGGCTCCCAGGCTCCTCATCTATT |
| | | GGGCATCCACTAGGGAATCTGGGG |
| | | TCCCCTCGAGGTTCAGTGGCAGTG |
| | | GATCTGGGACAGATTTCACCTTTAC |
| | | CATCAGTAGCCTGGAAGCTGAAGA |
| | | TGCTGCAACATATTACTGTCAGAAT |
| | | GATTATAGTTATCCGTACACGTTCG |
| | | GCCAAGGGACCAAGGTGGAAATCA |
| | | AA |
| SEQ ID NO: 202 | LC | EIVLTQSPDFQSVTPKEKVTIT |
| | | CKSSQSLLDSGNQKNFLTWYQQKPG |
| | | QAPRLLIYWASTRESGVPSRFSGSGS |
| | | GTDFTFTISSLEAEDAATYYCQNDYS |
| | | YPYTFGQGTKVEIKRTVAAPSVFIFPP |
| | | SDEQLKSGTASVVCLLNNFYPREAK |
| | | VQWKVDNALQSGNSQESVTEQDSK |
| | | DSTYSLSSTLTLSKADYEKHKVYAC |
| | | EVTHQGLSSPVTKSFNRGEC |
| SEQ ID NO: 203 | DNA LC | GAAATTGTGCTGACTCAGTC |
| | | TCCAGACTTTCAGTCTGTGACTCCA |
| | | AAGGAGAAAGTCACCATCACCTGC |
| | | AAGTCCAGTCAGAGTCTGTTAGAC |
| | | AGTGGAAATCAAAAGAACTTCTTG |
| | | ACCTGGTACCAGCAGAAACCTGGC |
| | | CAGGCTCCCAGGCTCCTCATCTATT |
| | | GGGCATCCACTAGGGAATCTGGGG |
| | | TCCCCTCGAGGTTCAGTGGCAGTG |
| | | GATCTGGGACAGATTTCACCTTTAC |
| | | CATCAGTAGCCTGGAAGCTGAAGA |
| | | TGCTGCAACATATTACTGTCAGAAT |
| | | GATTATAGTTATCCGTACACGTTCG |
| | | GCCAAGGGACCAAGGTGGAAATCA |
| | | AACGTACGGTGGCTGCACCATCTG |
| | | TCTTCATCTTCCCGCCATCTGATGA |
| | | GCAGTTGAAATCTGGAACTGCCTCT |
| | | GTTGTGTGCCTGCTGAATAACTTCT |
| | | ATCCCAGAGAGGCCAAAGTACAGT |
| | | GGAAGGTGGATAACGCCCTCCAAT |
| | | CGGGTAACTCCCAGGAGAGTGTCA |
| | | CAGAGCAGGACAGCAAGGACAGC |
| | | ACCTACAGCCTCAGCAGCACCCTG |
| | | ACGCTGAGCAAAGCAGACTACGAG |
| | | AAACACAAAGTCTACGCCTGCGAA |
| | | GTCACCCATCAGGGCCTGAGCTCG |
| | | CCCGTCACAAAGAGCTTCAACAGG |
| | | GGAGAGTGT |
| BAP049-hum09 HC | | |
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 | HCDR1 | GYTFTTY |
| (Chothia) | | |
| SEQ ID NO: 141 | HCDR2 | YPGTGG |
| (Chothia) | | |
| SEQ ID NO: 139 | HCDR3 | WTTGTGAY |
| (Chothia) | | |
| SEQ ID NO: 172 | VH | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSS |
| SEQ ID NO: 173 | DNA VH | GAAGTGCAGCTGGTGCAGTC |
| | | TGGAGCAGAGGTGAAAAAGCCCGG |
| | | GGAGTCTCTGAGGATCTCCTGTAA |
| | | GGGTTCTGGCTACACATTCACCACT |
| | | TACTGGATGCACTGGGTGCGACAG |
| | | GCCACTGGACAAGGGCTTGAGTGG |
| | | ATGGGTAATATTTATCCTGGTACTG |
| | | GTGGTTCTAACTTCGATGAGAAGTT |
| | | CAAGAACAGAGTCACGATTACCGC |
| | | GGACAAATCCACGAGCACAGCCTA |
| | | CATGGAGCTGAGCAGCCTGAGATC |
| | | TGAGGACACGGCCGTGTATTACTG |
| | | TACAAGATGGACTACTGGGACGGG |
| | | AGCTTATTGGGGCCAGGGCACCAC |
| | | CGTGACCGTGTCCTCC |
| SEQ ID NO: 174 | HC | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSSAS |
| | | TKGPSVFPLAPCSRSTSESTAALGCL |
| | | VKDYFPEPVTVSWNSGALTSGVHTF |
| | | PAVLQSSGLYSLSSVVTVPSSSLGTK |
| | | TYTCNVDHKPSNTKVDKRVESKYGP |
| | | PCPPCPAPEFLGGPSVFLFPPKPKDTL |
| | | MISRTPEVTCVVVDVSQEDPEVQFN |
| | | WYVDGVEVHNAKTKPREEQFNSTY |
| | | RVVSVLTVLHQDWLNGKEYKCKVS |
| | | NKGLPSSIEKTISKAKGQPREPQVYT |
| | | LPPSQEEMTKNQVSLTCLVKGFYPSD |
| | | IAVEWESNGQPENNYKTTPPVLDSD |
| | | GSFFLYSRLTVDKSRWQEGNVFSCS |
| | | VMHEALHNHYTQKSLSLSLGK |
| SEQ ID NO: 175 | DNA HC | GAAGTGCAGCTGGTGCAGTC |
| | | TGGAGCAGAGGTGAAAAAGCCCGG |
| | | GGAGTCTCTGAGGATCTCCTGTAA |
| | | GGGTTCTGGCTACACATTCACCACT |
| | | TACTGGATGCACTGGGTGCGACAG |
| | | GCCACTGGACAAGGGCTTGAGTGG |
| | | ATGGGTAATATTTATCCTGGTACTG |
| | | GTGGTTCTAACTTCGATGAGAAGTT |
| | | CAAGAACAGAGTCACGATTACCGC |
| | | GGACAAATCCACGAGCACAGCCTA |
| | | CATGGAGCTGAGCAGCCTGAGATC |
| | | TGAGGACACGGCCGTGTATTACTG |
| | | TACAAGATGGACTACTGGGACGGG |
| | | AGCTTATTGGGGCCAGGGCACCAC |
| | | CGTGACCGTGTCCTCCGCTTCCACC |
| | | AAGGGCCCATCCGTCTTCCCCCTGG |
| | | CGCCCTGCTCCAGGAGCACCTCCG |
| | | AGAGCACAGCCGCCCTGGGCTGCC |
| | | TGGTCAAGGACTACTTCCCCGAAC |
| | | CGGTGACGGTGTCGTGGAACTCAG |
| | | GCGCCCTGACCAGCGGCGTGCACA |
| | | CCTTCCCGGCTGTCCTACAGTCCTC |
| | | AGGACTCTACTCCCTCAGCAGCGT |
| | | GGTGACCGTGCCCTCCAGCAGCTT |
| | | GGGCACGAAGACCTACACCTGCAA |
| | | CGTAGATCACAAGCCCAGCAACAC |
| | | CAAGGTGGACAAGAGAGTTGAGTC |
| | | CAAATATGGTCCCCCATGCCCACC |
| | | GTGCCCAGCACCTGAGTTCCTGGG |
| | | GGGACCATCAGTCTTCCTGTTCCCC |
| | | CCAAAACCCAAGGACACTCTCATG |
| | | ATCTCCCGGACCCCTGAGGTCACGT |
| | | GCGTGGTGGTGGACGTGAGCCAGG |
| | | AAGACCCCGAGGTCCAGTTCAACT |
| | | GGTACGTGGATGGCGTGGAGGTGC |
| | | ATAATGCCAAGACAAAGCCGCGGG |
| | | AGGAGCAGTTCAACAGCACGTACC |
| | | GTGTGGTCAGCGTCCTCACCGTCCT |
| | | GCACCAGGACTGGCTGAACGGCAA |
| | | GGAGTACAAGTGCAAGGTGTCCAA |
| | | CAAAGGCCTCCCGTCCTCCATCGA |
| | | GAAAACCATCTCCAAAGCCAAAGG |
| | | GCAGCCCCGAGAGCCACAGGTGTA |
| | | CACCCTGCCCCCATCCCAGGAGGA |
| | | GATGACCAAGAACCAGGTCAGCCT |
| | | GACCTGCCTGGTCAAAGGCTTCTAC |
| | | CCCAGCGACATCGCCGTGGAGTGG |
| | | GAGAGCAATGGGCAGCCGGAGAAC |
| | | AACTACAAGACCACGCCTCCCGTG |
| | | CTGGACTCCGACGGCTCCTTCTTCC |
| | | TCTACAGCAGGCTAACCGTGGACA |
| | | AGAGCAGGTGGCAGGAGGGGAAT |
| | | GTCTTCTCATGCTCCGTGATGCATG |
| | | AGGCTCTGCACAACCACTACACAC |
| | | AGAAGAGCCTCTCCCTGTCTCTGGG |
| | | TAAA |
| BAP049-hum09 LC | | |
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 | LCDR1 | SQSLLDSGNQKNF |
| (Chothia) | | |
| SEQ ID NO: 150 | LCDR2 | WAS |
| (Chothia) | | |
| SEQ ID NO: 167 | LCDR3 | DYSYPY |
| (Chothia) | | |
| SEQ ID NO: 200 | VL | EIVLTQSPDFQSVTPKEKVTIT |
| | | CKSSQSLLDSGNQKNFLTWYQQKPG |
| | | QAPRLLIYWASTRESGVPSRFSGSGS |
| | | GTDFTFTISSLEAEDAATYYCQNDYS |
| | | YPYTFGQGTKVEIK |
| SEQ ID NO: 201 | DNA VL | GAAATTGTGCTGACTCAGTC |
| | | TCCAGACTTTCAGTCTGTGACTCCA |
| | | AAGGAGAAAGTCACCATCACCTGC |
| | | AAGTCCAGTCAGAGTCTGTTAGAC |
| | | AGTGGAAATCAAAAGAACTTCTTG |
| | | ACCTGGTACCAGCAGAAACCTGGC |
| | | CAGGCTCCCAGGCTCCTCATCTATT |
| | | GGGCATCCACTAGGGAATCTGGGG |
| | | TCCCCTCGAGGTTCAGTGGCAGTG |
| | | GATCTGGGACAGATTTCACCTTTAC |
| | | CATCAGTAGCCTGGAAGCTGAAGA |
| | | TGCTGCAACATATTACTGTCAGAAT |
| | | GATTATAGTTATCCGTACACGTTCG |
| | | GCCAAGGGACCAAGGTGGAAATCA |
| | | AA |
| SEQ ID NO: 202 | LC | EIVLTQSPDFQSVTPKEKVTIT |
| | | CKSSQSLLDSGNQKNFLTWYQQKPG |
| | | QAPRLLIYWASTRESGVPSRFSGSGS |
| | | GTDFTFTISSLEAEDAATYYCQNDYS |
| | | YPYTFGQGTKVEIKRTVAAPSVFIFPP |
| | | SDEQLKSGTASVVCLLNNFYPREAK |
| | | VQWKVDNALQSGNSQESVTEQDSK |
| | | DSTYSLSSTLTLSKADYEKHKVYAC |
| | | EVTHQGLSSPVTKSFNRGEC |
| SEQ ID NO: 203 | DNA LC | GAAATTGTGCTGACTCAGTC |
| | | TCCAGACTTTCAGTCTGTGACTCCA |
| | | AAGGAGAAAGTCACCATCACCTGC |
| | | AAGTCCAGTCAGAGTCTGTTAGAC |
| | | AGTGGAAATCAAAAGAACTTCTTG |
| | | ACCTGGTACCAGCAGAAACCTGGC |
| | | CAGGCTCCCAGGCTCCTCATCTATT |
| | | GGGCATCCACTAGGGAATCTGGGG |
| | | TCCCCTCGAGGTTCAGTGGCAGTG |
| | | GATCTGGGACAGATTTCACCTTTAC |
| | | CATCAGTAGCCTGGAAGCTGAAGA |
| | | TGCTGCAACATATTACTGTCAGAAT |
| | | GATTATAGTTATCCGTACACGTTCG |
| | | GCCAAGGGACCAAGGTGGAAATCA |
| | | AACGTACGGTGGCTGCACCATCTG |
| | | TCTTCATCTTCCCGCCATCTGATGA |
| | | GCAGTTGAAATCTGGAACTGCCTCT |
| | | GTTGTGTGCCTGCTGAATAACTTCT |
| | | ATCCCAGAGAGGCCAAAGTACAGT |
| | | GGAAGGTGGATAACGCCCTCCAAT |
| | | CGGGTAACTCCCAGGAGAGTGTCA |
| | | CAGAGCAGGACAGCAAGGACAGC |
| | | ACCTACAGCCTCAGCAGCACCCTG |
| | | ACGCTGAGCAAAGCAGACTACGAG |
| | | AAACACAAAGTCTACGCCTGCGAA |
| | | GTCACCCATCAGGGCCTGAGCTCG |
| | | CCCGTCACAAAGAGCTTCAACAGG |
| | | GGAGAGTGT |
| BAP049-hum10 HC | | |
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 | HCDR1 | GYTFTTY |
| (Chothia) | | |
| SEQ ID NO: 141 | HCDR2 | YPGTGG |
| (Chothia) | | |
| SEQ ID NO: 139 | HCDR3 | WTTGTGAY |
| (Chothia) | | |
| SEQ ID NO: 184 | VH | EVQLVQSGAEVKKPGESLRIS |
| | | CKGSGYTFTTYWMHWIRQSPSRGLE |
| | | WLGNIYPGTGGSNFDEKFKNRFTISR |
| | | DNSKNTLYLQMNSLRAEDTAVYYC |
| | | TRWTTGTGAYWGQGTTVTVSS |
| SEQ ID NO: 185 | DNA VH | GAAGTGCAGCTGGTGCAGTC |
| | | TGGAGCAGAGGTGAAAAAGCCCGG |
| | | GGAGTCTCTGAGGATCTCCTGTAA |
| | | GGGTTCTGGCTACACATTCACCACT |
| | | TACTGGATGCACTGGATCAGGCAG |
| | | TCCCCATCGAGAGGCCTTGAGTGG |
| | | CTGGGTAATATTTATCCTGGTACTG |
| | | GTGGTTCTAACTTCGATGAGAAGTT |
| | | CAAGAACAGATTCACCATCTCCAG |
| | | AGACAATTCCAAGAACACGCTGTA |
| | | TCTTCAAATGAACAGCCTGAGAGC |
| | | CGAGGACACGGCCGTGTATTACTG |
| | | TACAAGATGGACTACTGGGACGGG |
| | | AGCTTATTGGGGCCAGGGCACCAC |
| | | CGTGACCGTGTCCTCC |
| SEQ ID NO: 186 | HC | EVQLVQSGAEVKKPGESLRIS |
| | | CKGSGYTFTTYWMHWIRQSPSRGLE |
| | | WLGNIYPGTGGSNFDEKFKNRFTISR |
| | | DNSKNTLYLQMNSLRAEDTAVYYC |
| | | TRWTTGTGAYWGQGTTVTVSSAST |
| | | KGPSVFPLAPCSRSTSESTAALGCLV |
| | | KDYFPEPVTVSWNSGALTSGVHTFP |
| | | AVLQSSGLYSLSSVVTVPSSSLGTKT |
| | | YTCNVDHKPSNTKVDKRVESKYGPP |
| | | CPPCPAPEFLGGPSVFLFPPKPKDTL |
| | | MISRTPEVTCVVVDVSQEDPEVQFN |
| | | WYVDGVEVHNAKTKPREEQFNSTY |
| | | RVVSVLTVLHQDWLNGKEYKCKVS |
| | | NKGLPSSIEKTISKAKGQPREPQVYT |
| | | LPPSQEEMTKNQVSLTCLVKGFYPSD |
| | | IAVEWESNGQPENNYKTTPPVLDSD |
| | | GSFFLYSRLTVDKSRWQEGNVFSCS |
| | | VMHEALHNHYTQKSLSLSLGK |
| SEQ ID NO: 187 | DNA HC | GAAGTGCAGCTGGTGCAGTC |
| | | TGGAGCAGAGGTGAAAAAGCCCGG |
| | | GGAGTCTCTGAGGATCTCCTGTAA |
| | | GGGTTCTGGCTACACATTCACCACT |
| | | TACTGGATGCACTGGATCAGGCAG |
| | | TCCCCATCGAGAGGCCTTGAGTGG |
| | | CTGGGTAATATTTATCCTGGTACTG |
| | | GTGGTTCTAACTTCGATGAGAAGTT |
| | | CAAGAACAGATTCACCATCTCCAG |
| | | AGACAATTCCAAGAACACGCTGTA |
| | | TCTTCAAATGAACAGCCTGAGAGC |
| | | CGAGGACACGGCCGTGTATTACTG |
| | | TACAAGATGGACTACTGGGACGGG |
| | | AGCTTATTGGGGCCAGGGCACCAC |
| | | CGTGACCGTGTCCTCCGCTTCCACC |
| | | AAGGGCCCATCCGTCTTCCCCCTGG |
| | | CGCCCTGCTCCAGGAGCACCTCCG |
| | | AGAGCACAGCCGCCCTGGGCTGCC |
| | | TGGTCAAGGACTACTTCCCCGAAC |
| | | CGGTGACGGTGTCGTGGAACTCAG |
| | | GCGCCCTGACCAGCGGCGTGCACA |
| | | CCTTCCCGGCTGTCCTACAGTCCTC |
| | | AGGACTCTACTCCCTCAGCAGCGT |
| | | GGTGACCGTGCCCTCCAGCAGCTT |
| | | GGGCACGAAGACCTACACCTGCAA |
| | | CGTAGATCACAAGCCCAGCAACAC |
| | | CAAGGTGGACAAGAGAGTTGAGTC |
| | | CAAATATGGTCCCCCATGCCCACC |
| | | GTGCCCAGCACCTGAGTTCCTGGG |
| | | GGGACCATCAGTCTTCCTGTTCCCC |
| | | CCAAAACCCAAGGACACTCTCATG |
| | | ATCTCCCGGACCCCTGAGGTCACGT |
| | | GCGTGGTGGTGGACGTGAGCCAGG |
| | | AAGACCCCGAGGTCCAGTTCAACT |
| | | GGTACGTGGATGGCGTGGAGGTGC |
| | | ATAATGCCAAGACAAAGCCGCGGG |
| | | AGGAGCAGTTCAACAGCACGTACC |
| | | GTGTGGTCAGCGTCCTCACCGTCCT |
| | | GCACCAGGACTGGCTGAACGGCAA |
| | | GGAGTACAAGTGCAAGGTGTCCAA |
| | | CAAAGGCCTCCCGTCCTCCATCGA |
| | | GAAAACCATCTCCAAAGCCAAAGG |
| | | GCAGCCCCGAGAGCCACAGGTGTA |
| | | CACCCTGCCCCCATCCCAGGAGGA |
| | | GATGACCAAGAACCAGGTCAGCCT |
| | | GACCTGCCTGGTCAAAGGCTTCTAC |
| | | CCCAGCGACATCGCCGTGGAGTGG |
| | | GAGAGCAATGGGCAGCCGGAGAAC |
| | | AACTACAAGACCACGCCTCCCGTG |
| | | CTGGACTCCGACGGCTCCTTCTTCC |
| | | TCTACAGCAGGCTAACCGTGGACA |
| | | AGAGCAGGTGGCAGGAGGGGAAT |
| | | GTCTTCTCATGCTCCGTGATGCATG |
| | | AGGCTCTGCACAACCACTACACAC |
| | | AGAAGAGCCTCTCCCTGTCTCTGGG |
| | | TAAA |
| BAP049-hum10 LC | | |
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 | LCDR1 | SQSLLDSGNQKNF |
| (Chothia) | | |
| SEQ ID NO: 150 | LCDR2 | WAS |
| (Chothia) | | |
| SEQ ID NO: 167 | LCDR3 | DYSYPY |
| (Chothia) | | |
| SEQ ID NO: 204 | VL | EIVLTQSPATLSLSPGERATLS |
| | | CKSSQSLLDSGNQKNFLTWYQQKPG |
| | | QAPRLLIYWASTRESGVPSRFSGSGS |
| | | GTDFTFTISSLEAEDAATYYCQNDYS |
| | | YPYTFGQGTKVEIK |
| SEQ ID NO: 205 | DNA VL | GAAATTGTGTTGACACAGTC |
| | | TCCAGCCACCCTGTCTTTGTCTCCA |
| | | GGGGAAAGAGCCACCCTCTCCTGC |
| | | AAGTCCAGTCAGAGTCTGTTAGAC |
| | | AGTGGAAATCAAAAGAACTTCTTG |
| | | ACCTGGTACCAGCAGAAACCTGGC |
| | | CAGGCTCCCAGGCTCCTCATCTATT |
| | | GGGCATCCACTAGGGAATCTGGGG |
| | | TCCCCTCGAGGTTCAGTGGCAGTG |
| | | GATCTGGGACAGATTTCACCTTTAC |
| | | CATCAGTAGCCTGGAAGCTGAAGA |
| | | TGCTGCAACATATTACTGTCAGAAT |
| | | GATTATAGTTATCCGTACACGTTCG |
| | | GCCAAGGGACCAAGGTGGAAATCA |
| | | AA |
| SEQ ID NO: 206 | LC | EIVLTQSPATLSLSPGERATLS |
| | | CKSSQSLLDSGNQKNFLTWYQQKPG |
| | | QAPRLLIYWASTRESGVPSRFSGSGS |
| | | GTDFTFTISSLEAEDAATYYCQNDYS |
| | | YPYTFGQGTKVEIKRTVAAPSVFIFPP |
| | | SDEQLKSGTASVVCLLNNFYPREAK |
| | | VQWKVDNALQSGNSQESVTEQDSK |
| | | DSTYSLSSTLTLSKADYEKHKVYAC |
| | | EVTHQGLSSPVTKSFNRGEC |
| SEQ ID NO: 207 | DNA LC | GAAATTGTGTTGACACAGTC |
| | | TCCAGCCACCCTGTCTTTGTCTCCA |
| | | GGGGAAAGAGCCACCCTCTCCTGC |
| | | AAGTCCAGTCAGAGTCTGTTAGAC |
| | | AGTGGAAATCAAAAGAACTTCTTG |
| | | ACCTGGTACCAGCAGAAACCTGGC |
| | | CAGGCTCCCAGGCTCCTCATCTATT |
| | | GGGCATCCACTAGGGAATCTGGGG |
| | | TCCCCTCGAGGTTCAGTGGCAGTG |
| | | GATCTGGGACAGATTTCACCTTTAC |
| | | CATCAGTAGCCTGGAAGCTGAAGA |
| | | TGCTGCAACATATTACTGTCAGAAT |
| | | GATTATAGTTATCCGTACACGTTCG |
| | | GCCAAGGGACCAAGGTGGAAATCA |
| | | AACGTACGGTGGCTGCACCATCTG |
| | | TCTTCATCTTCCCGCCATCTGATGA |
| | | GCAGTTGAAATCTGGAACTGCCTCT |
| | | GTTGTGTGCCTGCTGAATAACTTCT |
| | | ATCCCAGAGAGGCCAAAGTACAGT |
| | | GGAAGGTGGATAACGCCCTCCAAT |
| | | CGGGTAACTCCCAGGAGAGTGTCA |
| | | CAGAGCAGGACAGCAAGGACAGC |
| | | ACCTACAGCCTCAGCAGCACCCTG |
| | | ACGCTGAGCAAAGCAGACTACGAG |
| | | AAACACAAAGTCTACGCCTGCGAA |
| | | GTCACCCATCAGGGCCTGAGCTCG |
| | | CCCGTCACAAAGAGCTTCAACAGG |
| | | GGAGAGTGT |
| BAP049-hum11 HC | | |
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 | HCDR1 | GYTFTTY |
| (Chothia) | | |
| SEQ ID NO: 141 | HCDR2 | YPGTGG |
| (Chothia) | | |
| SEQ ID NO: 139 | HCDR3 | WTTGTGAY |
| (Chothia) | | |
| SEQ ID NO: 172 | VH | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSS |
| SEQ ID NO: 173 | DNA VH | GAAGTGCAGCTGGTGCAGTC |
| | | TGGAGCAGAGGTGAAAAAGCCCGG |
| | | GGAGTCTCTGAGGATCTCCTGTAA |
| | | GGGTTCTGGCTACACATTCACCACT |
| | | TACTGGATGCACTGGGTGCGACAG |
| | | GCCACTGGACAAGGGCTTGAGTGG |
| | | ATGGGTAATATTTATCCTGGTACTG |
| | | GTGGTTCTAACTTCGATGAGAAGTT |
| | | CAAGAACAGAGTCACGATTACCGC |
| | | GGACAAATCCACGAGCACAGCCTA |
| | | CATGGAGCTGAGCAGCCTGAGATC |
| | | TGAGGACACGGCCGTGTATTACTG |
| | | TACAAGATGGACTACTGGGACGGG |
| | | AGCTTATTGGGGCCAGGGCACCAC |
| | | CGTGACCGTGTCCTCC |
| SEQ ID NO: 174 | HC | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSSAS |
| | | TKGPSVFPLAPCSRSTSESTAALGCL |
| | | VKDYFPEPVTVSWNSGALTSGVHTF |
| | | PAVLQSSGLYSLSSVVTVPSSSLGTK |
| | | TYTCNVDHKPSNTKVDKRVESKYGP |
| | | PCPPCPAPEFLGGPSVFLFPPKPKDTL |
| | | MISRTPEVTCVVVDVSQEDPEVQFN |
| | | WYVDGVEVHNAKTKPREEQFNSTY |
| | | RVVSVLTVLHQDWLNGKEYKCKVS |
| | | NKGLPSSIEKTISKAKGQPREPQVYT |
| | | LPPSQEEMTKNQVSLTCLVKGFYPSD |
| | | IAVEWESNGQPENNYKTTPPVLDSD |
| | | GSFFLYSRLTVDKSRWQEGNVFSCS |
| | | VMHEALHNHYTQKSLSLSLGK |
| SEQ ID NO: 175 | DNA HC | GAAGTGCAGCTGGTGCAGTC |
| | | TGGAGCAGAGGTGAAAAAGCCCGG |
| | | GGAGTCTCTGAGGATCTCCTGTAA |
| | | GGGTTCTGGCTACACATTCACCACT |
| | | TACTGGATGCACTGGGTGCGACAG |
| | | GCCACTGGACAAGGGCTTGAGTGG |
| | | ATGGGTAATATTTATCCTGGTACTG |
| | | GTGGTTCTAACTTCGATGAGAAGTT |
| | | CAAGAACAGAGTCACGATTACCGC |
| | | GGACAAATCCACGAGCACAGCCTA |
| | | CATGGAGCTGAGCAGCCTGAGATC |
| | | TGAGGACACGGCCGTGTATTACTG |
| | | TACAAGATGGACTACTGGGACGGG |
| | | AGCTTATTGGGGCCAGGGCACCAC |
| | | CGTGACCGTGTCCTCCGCTTCCACC |
| | | AAGGGCCCATCCGTCTTCCCCCTGG |
| | | CGCCCTGCTCCAGGAGCACCTCCG |
| | | AGAGCACAGCCGCCCTGGGCTGCC |
| | | TGGTCAAGGACTACTTCCCCGAAC |
| | | CGGTGACGGTGTCGTGGAACTCAG |
| | | GCGCCCTGACCAGCGGCGTGCACA |
| | | CCTTCCCGGCTGTCCTACAGTCCTC |
| | | AGGACTCTACTCCCTCAGCAGCGT |
| | | GGTGACCGTGCCCTCCAGCAGCTT |
| | | GGGCACGAAGACCTACACCTGCAA |
| | | CGTAGATCACAAGCCCAGCAACAC |
| | | CAAGGTGGACAAGAGAGTTGAGTC |
| | | CAAATATGGTCCCCCATGCCCACC |
| | | GTGCCCAGCACCTGAGTTCCTGGG |
| | | GGGACCATCAGTCTTCCTGTTCCCC |
| | | CCAAAACCCAAGGACACTCTCATG |
| | | ATCTCCCGGACCCCTGAGGTCACGT |
| | | GCGTGGTGGTGGACGTGAGCCAGG |
| | | AAGACCCCGAGGTCCAGTTCAACT |
| | | GGTACGTGGATGGCGTGGAGGTGC |
| | | ATAATGCCAAGACAAAGCCGCGGG |
| | | AGGAGCAGTTCAACAGCACGTACC |
| | | GTGTGGTCAGCGTCCTCACCGTCCT |
| | | GCACCAGGACTGGCTGAACGGCAA |
| | | GGAGTACAAGTGCAAGGTGTCCAA |
| | | CAAAGGCCTCCCGTCCTCCATCGA |
| | | GAAAACCATCTCCAAAGCCAAAGG |
| | | GCAGCCCCGAGAGCCACAGGTGTA |
| | | CACCCTGCCCCCATCCCAGGAGGA |
| | | GATGACCAAGAACCAGGTCAGCCT |
| | | GACCTGCCTGGTCAAAGGCTTCTAC |
| | | CCCAGCGACATCGCCGTGGAGTGG |
| | | GAGAGCAATGGGCAGCCGGAGAAC |
| | | AACTACAAGACCACGCCTCCCGTG |
| | | CTGGACTCCGACGGCTCCTTCTTCC |
| | | TCTACAGCAGGCTAACCGTGGACA |
| | | AGAGCAGGTGGCAGGAGGGGAAT |
| | | GTCTTCTCATGCTCCGTGATGCATG |
| | | AGGCTCTGCACAACCACTACACAC |
| | | AGAAGAGCCTCTCCCTGTCTCTGGG |
| | | TAAA |
| BAP049-hum11 LC | | |
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 | LCDR1 | SQSLLDSGNQKNF |
| (Chothia) | | |
| SEQ ID NO: 150 | LCDR2 | WAS |
| (Chothia) | | |
| SEQ ID NO: 167 | LCDR3 | DYSYPY |
| (Chothia) | | |
| SEQ ID NO: 204 | VL | EIVLTQSPATLSLSPGERATLS |
| | | CKSSQSLLDSGNQKNFLTWYQQKPG |
| | | QAPRLLIYWASTRESGVPSRFSGSGS |
| | | GTDFTFTISSLEAEDAATYYCQNDYS |
| | | YPYTFGQGTKVEIK |
| SEQ ID NO: 205 | DNA VL | GAAATTGTGTTGACACAGTC |
| | | TCCAGCCACCCTGTCTTTGTCTCCA |
| | | GGGGAAAGAGCCACCCTCTCCTGC |
| | | AAGTCCAGTCAGAGTCTGTTAGAC |
| | | AGTGGAAATCAAAAGAACTTCTTG |
| | | ACCTGGTACCAGCAGAAACCTGGC |
| | | CAGGCTCCCAGGCTCCTCATCTATT |
| | | GGGCATCCACTAGGGAATCTGGGG |
| | | TCCCCTCGAGGTTCAGTGGCAGTG |
| | | GATCTGGGACAGATTTCACCTTTAC |
| | | CATCAGTAGCCTGGAAGCTGAAGA |
| | | TGCTGCAACATATTACTGTCAGAAT |
| | | GATTATAGTTATCCGTACACGTTCG |
| | | GCCAAGGGACCAAGGTGGAAATCA |
| | | AA |
| SEQ ID NO: 206 | LC | EIVLTQSPATLSLSPGERATLS |
| | | CKSSQSLLDSGNQKNFLTWYQQKPG |
| | | QAPRLLIYWASTRESGVPSRFSGSGS |
| | | GTDFTFTISSLEAEDAATYYCQNDYS |
| | | YPYTFGQGTKVEIKRTVAAPSVFIFPP |
| | | SDEQLKSGTASVVCLLNNFYPREAK |
| | | VQWKVDNALQSGNSQESVTEQDSK |
| | | DSTYSLSSTLTLSKADYEKHKVYAC |
| | | EVTHQGLSSPVTKSFNRGEC |
| SEQ ID NO: 207 | DNA LC | GAAATTGTGTTGACACAGTC |
| | | TCCAGCCACCCTGTCTTTGTCTCCA |
| | | GGGGAAAGAGCCACCCTCTCCTGC |
| | | AAGTCCAGTCAGAGTCTGTTAGAC |
| | | AGTGGAAATCAAAAGAACTTCTTG |
| | | ACCTGGTACCAGCAGAAACCTGGC |
| | | CAGGCTCCCAGGCTCCTCATCTATT |
| | | GGGCATCCACTAGGGAATCTGGGG |
| | | TCCCCTCGAGGTTCAGTGGCAGTG |
| | | GATCTGGGACAGATTTCACCTTTAC |
| | | CATCAGTAGCCTGGAAGCTGAAGA |
| | | TGCTGCAACATATTACTGTCAGAAT |
| | | GATTATAGTTATCCGTACACGTTCG |
| | | GCCAAGGGACCAAGGTGGAAATCA |
| | | AACGTACGGTGGCTGCACCATCTG |
| | | TCTTCATCTTCCCGCCATCTGATGA |
| | | GCAGTTGAAATCTGGAACTGCCTCT |
| | | GTTGTGTGCCTGCTGAATAACTTCT |
| | | ATCCCAGAGAGGCCAAAGTACAGT |
| | | GGAAGGTGGATAACGCCCTCCAAT |
| | | CGGGTAACTCCCAGGAGAGTGTCA |
| | | CAGAGCAGGACAGCAAGGACAGC |
| | | ACCTACAGCCTCAGCAGCACCCTG |
| | | ACGCTGAGCAAAGCAGACTACGAG |
| | | AAACACAAAGTCTACGCCTGCGAA |
| | | GTCACCCATCAGGGCCTGAGCTCG |
| | | CCCGTCACAAAGAGCTTCAACAGG |
| | | GGAGAGTGT |
| BAP049-hum12 HC | | |
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 | HCDR1 | GYTFTTY |
| (Chothia) | | |
| SEQ ID NO: 141 | HCDR2 | YPGTGG |
| (Chothia) | | |
| SEQ ID NO: 139 | HCDR3 | WTTGTGAY |
| (Chothia) | | |
| SEQ ID NO: 172 | VH | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSS |
| SEQ ID NO: 173 | DNA VH | GAAGTGCAGCTGGTGCAGTC |
| | | TGGAGCAGAGGTGAAAAAGCCCGG |
| | | GGAGTCTCTGAGGATCTCCTGTAA |
| | | GGGTTCTGGCTACACATTCACCACT |
| | | TACTGGATGCACTGGGTGCGACAG |
| | | GCCACTGGACAAGGGCTTGAGTGG |
| | | ATGGGTAATATTTATCCTGGTACTG |
| | | GTGGTTCTAACTTCGATGAGAAGTT |
| | | CAAGAACAGAGTCACGATTACCGC |
| | | GGACAAATCCACGAGCACAGCCTA |
| | | CATGGAGCTGAGCAGCCTGAGATC |
| | | TGAGGACACGGCCGTGTATTACTG |
| | | TACAAGATGGACTACTGGGACGGG |
| | | AGCTTATTGGGGCCAGGGCACCAC |
| | | CGTGACCGTGTCCTCC |
| SEQ ID NO: 174 | HC | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSSAS |
| | | TKGPSVFPLAPCSRSTSESTAALGCL |
| | | VKDYFPEPVTVSWNSGALTSGVHTF |
| | | PAVLQSSGLYSLSSVVTVPSSSLGTK |
| | | TYTCNVDHKPSNTKVDKRVESKYGP |
| | | PCPPCPAPEFLGGPSVFLFPPKPKDTL |
| | | MISRTPEVTCVVVDVSQEDPEVQFN |
| | | WYVDGVEVHNAKTKPREEQFNSTY |
| | | RVVSVLTVLHQDWLNGKEYKCKVS |
| | | NKGLPSSIEKTISKAKGQPREPQVYT |
| | | LPPSQEEMTKNQVSLTCLVKGFYPSD |
| | | IAVEWESNGQPENNYKTTPPVLDSD |
| | | GSFFLYSRLTVDKSRWQEGNVFSCS |
| | | VMHEALHNHYTQKSLSLSLGK |
| SEQ ID NO: 175 | DNA HC | GAAGTGCAGCTGGTGCAGTC |
| | | TGGAGCAGAGGTGAAAAAGCCCGG |
| | | GGAGTCTCTGAGGATCTCCTGTAA |
| | | GGGTTCTGGCTACACATTCACCACT |
| | | TACTGGATGCACTGGGTGCGACAG |
| | | GCCACTGGACAAGGGCTTGAGTGG |
| | | ATGGGTAATATTTATCCTGGTACTG |
| | | GTGGTTCTAACTTCGATGAGAAGTT |
| | | CAAGAACAGAGTCACGATTACCGC |
| | | GGACAAATCCACGAGCACAGCCTA |
| | | CATGGAGCTGAGCAGCCTGAGATC |
| | | TGAGGACACGGCCGTGTATTACTG |
| | | TACAAGATGGACTACTGGGACGGG |
| | | AGCTTATTGGGGCCAGGGCACCAC |
| | | CGTGACCGTGTCCTCCGCTTCCACC |
| | | AAGGGCCCATCCGTCTTCCCCCTGG |
| | | CGCCCTGCTCCAGGAGCACCTCCG |
| | | AGAGCACAGCCGCCCTGGGCTGCC |
| | | TGGTCAAGGACTACTTCCCCGAAC |
| | | CGGTGACGGTGTCGTGGAACTCAG |
| | | GCGCCCTGACCAGCGGCGTGCACA |
| | | CCTTCCCGGCTGTCCTACAGTCCTC |
| | | AGGACTCTACTCCCTCAGCAGCGT |
| | | GGTGACCGTGCCCTCCAGCAGCTT |
| | | GGGCACGAAGACCTACACCTGCAA |
| | | CGTAGATCACAAGCCCAGCAACAC |
| | | CAAGGTGGACAAGAGAGTTGAGTC |
| | | CAAATATGGTCCCCCATGCCCACC |
| | | GTGCCCAGCACCTGAGTTCCTGGG |
| | | GGGACCATCAGTCTTCCTGTTCCCC |
| | | CCAAAACCCAAGGACACTCTCATG |
| | | ATCTCCCGGACCCCTGAGGTCACGT |
| | | GCGTGGTGGTGGACGTGAGCCAGG |
| | | AAGACCCCGAGGTCCAGTTCAACT |
| | | GGTACGTGGATGGCGTGGAGGTGC |
| | | ATAATGCCAAGACAAAGCCGCGGG |
| | | AGGAGCAGTTCAACAGCACGTACC |
| | | GTGTGGTCAGCGTCCTCACCGTCCT |
| | | GCACCAGGACTGGCTGAACGGCAA |
| | | GGAGTACAAGTGCAAGGTGTCCAA |
| | | CAAAGGCCTCCCGTCCTCCATCGA |
| | | GAAAACCATCTCCAAAGCCAAAGG |
| | | GCAGCCCCGAGAGCCACAGGTGTA |
| | | CACCCTGCCCCCATCCCAGGAGGA |
| | | GATGACCAAGAACCAGGTCAGCCT |
| | | GACCTGCCTGGTCAAAGGCTTCTAC |
| | | CCCAGCGACATCGCCGTGGAGTGG |
| | | GAGAGCAATGGGCAGCCGGAGAAC |
| | | AACTACAAGACCACGCCTCCCGTG |
| | | CTGGACTCCGACGGCTCCTTCTTCC |
| | | TCTACAGCAGGCTAACCGTGGACA |
| | | AGAGCAGGTGGCAGGAGGGGAAT |
| | | GTCTTCTCATGCTCCGTGATGCATG |
| | | AGGCTCTGCACAACCACTACACAC |
| | | AGAAGAGCCTCTCCCTGTCTCTGGG |
| | | TAAA |
| BAP049-hum12 LC | | |
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 | LCDR1 | SQSLLDSGNQKNF |
| (Chothia) | | |
| SEQ ID NO: 150 | LCDR2 | WAS |
| (Chothia) | | |
| SEQ ID NO: 167 | LCDR3 | DYSYPY |
| (Chothia) | | |
| SEQ ID NO: 208 | VL | DIQMTQSPSSLSASVGDRVTIT |
| | | CKSSQSLLDSGNQKNFLTWYLQKPG |
| | | QSPQLLIYWASTRESGVPSRFSGSGS |
| | | GTDFTFTISSLEAEDAATYYCQNDYS |
| | | YPYTFGQGTKVEIK |
| SEQ ID NO: 209 | DNA VL | GACATCCAGATGACCCAGTC |
| | | TCCATCCTCCCTGTCTGCATCTGTA |
| | | GGAGACAGAGTCACCATCACTTGC |
| | | AAGTCCAGTCAGAGTCTGTTAGAC |
| | | AGTGGAAATCAAAAGAACTTCTTG |
| | | ACCTGGTACCTGCAGAAGCCAGGG |
| | | CAGTCTCCACAGCTCCTGATCTATT |
| | | GGGCATCCACTAGGGAATCTGGGG |
| | | TCCCCTCGAGGTTCAGTGGCAGTG |
| | | GATCTGGGACAGATTTCACCTTTAC |
| | | CATCAGTAGCCTGGAAGCTGAAGA |
| | | TGCTGCAACATATTACTGTCAGAAT |
| | | GATTATAGTTATCCGTACACGTTCG |
| | | GCCAAGGGACCAAGGTGGAAATCA |
| | | AA |
| SEQ ID NO: 210 | LC | DIQMTQSPSSLSASVGDRVTIT |
| | | CKSSQSLLDSGNQKNFLTWYLQKPG |
| | | QSPQLLIYWASTRESGVPSRFSGSGS |
| | | GTDFTFTISSLEAEDAATYYCQNDYS |
| | | YPYTFGQGTKVEIKRTVAAPSVFIFPP |
| | | SDEQLKSGTASVVCLLNNFYPREAK |
| | | VQWKVDNALQSGNSQESVTEQDSK |
| | | DSTYSLSSTLTLSKADYEKHKVYAC |
| | | EVTHQGLSSPVTKSFNRGEC |
| SEQ ID NO: 211 | DNA LC | GACATCCAGATGACCCAGTC |
| | | TCCATCCTCCCTGTCTGCATCTGTA |
| | | GGAGACAGAGTCACCATCACTTGC |
| | | AAGTCCAGTCAGAGTCTGTTAGAC |
| | | AGTGGAAATCAAAAGAACTTCTTG |
| | | ACCTGGTACCTGCAGAAGCCAGGG |
| | | CAGTCTCCACAGCTCCTGATCTATT |
| | | GGGCATCCACTAGGGAATCTGGGG |
| | | TCCCCTCGAGGTTCAGTGGCAGTG |
| | | GATCTGGGACAGATTTCACCTTTAC |
| | | CATCAGTAGCCTGGAAGCTGAAGA |
| | | TGCTGCAACATATTACTGTCAGAAT |
| | | GATTATAGTTATCCGTACACGTTCG |
| | | GCCAAGGGACCAAGGTGGAAATCA |
| | | AACGTACGGTGGCTGCACCATCTG |
| | | TCTTCATCTTCCCGCCATCTGATGA |
| | | GCAGTTGAAATCTGGAACTGCCTCT |
| | | GTTGTGTGCCTGCTGAATAACTTCT |
| | | ATCCCAGAGAGGCCAAAGTACAGT |
| | | GGAAGGTGGATAACGCCCTCCAAT |
| | | CGGGTAACTCCCAGGAGAGTGTCA |
| | | CAGAGCAGGACAGCAAGGACAGC |
| | | ACCTACAGCCTCAGCAGCACCCTG |
| | | ACGCTGAGCAAAGCAGACTACGAG |
| | | AAACACAAAGTCTACGCCTGCGAA |
| | | GTCACCCATCAGGGCCTGAGCTCG |
| | | CCCGTCACAAAGAGCTTCAACAGG |
| | | GGAGAGTGT |
| BAP049-hum13 HC | | |
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 | HCDR1 | GYTFTTY |
| (Chothia) | | |
| SEQ ID NO: 141 | HCDR2 | YPGTGG |
| (Chothia) | | |
| SEQ ID NO: 139 | HCDR3 | WTTGTGAY |
| (Chothia) | | |
| SEQ ID NO: 172 | VH | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSS |
| SEQ ID NO: 173 | DNA VH | GAAGTGCAGCTGGTGCAGTC |
| | | TGGAGCAGAGGTGAAAAAGCCCGG |
| | | GGAGTCTCTGAGGATCTCCTGTAA |
| | | GGGTTCTGGCTACACATTCACCACT |
| | | TACTGGATGCACTGGGTGCGACAG |
| | | GCCACTGGACAAGGGCTTGAGTGG |
| | | ATGGGTAATATTTATCCTGGTACTG |
| | | GTGGTTCTAACTTCGATGAGAAGTT |
| | | CAAGAACAGAGTCACGATTACCGC |
| | | GGACAAATCCACGAGCACAGCCTA |
| | | CATGGAGCTGAGCAGCCTGAGATC |
| | | TGAGGACACGGCCGTGTATTACTG |
| | | TACAAGATGGACTACTGGGACGGG |
| | | AGCTTATTGGGGCCAGGGCACCAC |
| | | CGTGACCGTGTCCTCC |
| SEQ ID NO: 174 | HC | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSSAS |
| | | TKGPSVFPLAPCSRSTSESTAALGCL |
| | | VKDYFPEPVTVSWNSGALTSGVHTF |
| | | PAVLQSSGLYSLSSVVTVPSSSLGTK |
| | | TYTCNVDHKPSNTKVDKRVESKYGP |
| | | PCPPCPAPEFLGGPSVFLFPPKPKDTL |
| | | MISRTPEVTCVVVDVSQEDPEVQFN |
| | | WYVDGVEVHNAKTKPREEQFNSTY |
| | | RVVSVLTVLHQDWLNGKEYKCKVS |
| | | NKGLPSSIEKTISKAKGQPREPQVYT |
| | | LPPSQEEMTKNQVSLTCLVKGFYPSD |
| | | IAVEWESNGQPENNYKTTPPVLDSD |
| | | GSFFLYSRLTVDKSRWQEGNVFSCS |
| | | VMHEALHNHYTQKSLSLSLGK |
| SEQ ID NO: 175 | DNA HC | GAAGTGCAGCTGGTGCAGTC |
| | | TGGAGCAGAGGTGAAAAAGCCCGG |
| | | GGAGTCTCTGAGGATCTCCTGTAA |
| | | GGGTTCTGGCTACACATTCACCACT |
| | | TACTGGATGCACTGGGTGCGACAG |
| | | GCCACTGGACAAGGGCTTGAGTGG |
| | | ATGGGTAATATTTATCCTGGTACTG |
| | | GTGGTTCTAACTTCGATGAGAAGTT |
| | | CAAGAACAGAGTCACGATTACCGC |
| | | GGACAAATCCACGAGCACAGCCTA |
| | | CATGGAGCTGAGCAGCCTGAGATC |
| | | TGAGGACACGGCCGTGTATTACTG |
| | | TACAAGATGGACTACTGGGACGGG |
| | | AGCTTATTGGGGCCAGGGCACCAC |
| | | CGTGACCGTGTCCTCCGCTTCCACC |
| | | AAGGGCCCATCCGTCTTCCCCCTGG |
| | | CGCCCTGCTCCAGGAGCACCTCCG |
| | | AGAGCACAGCCGCCCTGGGCTGCC |
| | | TGGTCAAGGACTACTTCCCCGAAC |
| | | CGGTGACGGTGTCGTGGAACTCAG |
| | | GCGCCCTGACCAGCGGCGTGCACA |
| | | CCTTCCCGGCTGTCCTACAGTCCTC |
| | | AGGACTCTACTCCCTCAGCAGCGT |
| | | GGTGACCGTGCCCTCCAGCAGCTT |
| | | GGGCACGAAGACCTACACCTGCAA |
| | | CGTAGATCACAAGCCCAGCAACAC |
| | | CAAGGTGGACAAGAGAGTTGAGTC |
| | | CAAATATGGTCCCCCATGCCCACC |
| | | GTGCCCAGCACCTGAGTTCCTGGG |
| | | GGGACCATCAGTCTTCCTGTTCCCC |
| | | CCAAAACCCAAGGACACTCTCATG |
| | | ATCTCCCGGACCCCTGAGGTCACGT |
| | | GCGTGGTGGTGGACGTGAGCCAGG |
| | | AAGACCCCGAGGTCCAGTTCAACT |
| | | GGTACGTGGATGGCGTGGAGGTGC |
| | | ATAATGCCAAGACAAAGCCGCGGG |
| | | AGGAGCAGTTCAACAGCACGTACC |
| | | GTGTGGTCAGCGTCCTCACCGTCCT |
| | | GCACCAGGACTGGCTGAACGGCAA |
| | | GGAGTACAAGTGCAAGGTGTCCAA |
| | | CAAAGGCCTCCCGTCCTCCATCGA |
| | | GAAAACCATCTCCAAAGCCAAAGG |
| | | GCAGCCCCGAGAGCCACAGGTGTA |
| | | CACCCTGCCCCCATCCCAGGAGGA |
| | | GATGACCAAGAACCAGGTCAGCCT |
| | | GACCTGCCTGGTCAAAGGCTTCTAC |
| | | CCCAGCGACATCGCCGTGGAGTGG |
| | | GAGAGCAATGGGCAGCCGGAGAAC |
| | | AACTACAAGACCACGCCTCCCGTG |
| | | CTGGACTCCGACGGCTCCTTCTTCC |
| | | TCTACAGCAGGCTAACCGTGGACA |
| | | AGAGCAGGTGGCAGGAGGGGAAT |
| | | GTCTTCTCATGCTCCGTGATGCATG |
| | | AGGCTCTGCACAACCACTACACAC |
| | | AGAAGAGCCTCTCCCTGTCTCTGGG |
| | | TAAA |
| BAP049-hum13 LC | | |
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 | LCDR1 | SQSLLDSGNQKNF |
| (Chothia) | | |
| SEQ ID NO: 150 | LCDR2 | WAS |
| (Chothia) | | |
| SEQ ID NO: 167 | LCDR3 | DYSYPY |
| (Chothia) | | |
| SEQ ID NO: 212 | VL | DVVMTQSPLSLPVTLGQPASIS |
| | | CKSSQSLLDSGNQKNFLTWYQQKPG |
| | | KAPKLLIYWASTRESGVPSRFSGSGS |
| | | GTDFTFTISSLEAEDAATYYCQNDYS |
| | | YPYTFGQGTKVEIK |
| SEQ ID NO: 213 | DNA VL | GATGTTGTGATGACTCAGTC |
| | | TCCACTCTCCCTGCCCGTCACCCTT |
| | | GGACAGCCGGCCTCCATCTCCTGC |
| | | AAGTCCAGTCAGAGTCTGTTAGAC |
| | | AGTGGAAATCAAAAGAACTTCTTA |
| | | ACCTGGTATCAGCAGAAACCAGGG |
| | | AAAGCTCCTAAGCTCCTGATCTATT |
| | | GGGCATCCACTAGGGAATCTGGGG |
| | | TCCCCTCGAGGTTCAGTGGCAGTG |
| | | GATCTGGGACAGATTTCACCTTTAC |
| | | CATCAGTAGCCTGGAAGCTGAAGA |
| | | TGCTGCAACATATTACTGTCAGAAT |
| | | GATTATAGTTATCCGTACACGTTCG |
| | | GCCAAGGGACCAAGGTGGAAATCA |
| | | AA |
| SEQ ID NO: 214 | LC | DVVMTQSPLSLPVTLGQPASIS |
| | | CKSSQSLLDSGNQKNFLTWYQQKPG |
| | | KAPKLLIYWASTRESGVPSRFSGSGS |
| | | GTDFTFTISSLEAEDAATYYCQNDYS |
| | | YPYTFGQGTKVEIKRTVAAPSVFIFPP |
| | | SDEQLKSGTASVVCLLNNFYPREAK |
| | | VQWKVDNALQSGNSQESVTEQDSK |
| | | DSTYSLSSTLTLSKADYEKHKVYAC |
| | | EVTHQGLSSPVTKSFNRGEC |
| SEQ ID NO: 215 | DNA LC | GATGTTGTGATGACTCAGTC |
| | | TCCACTCTCCCTGCCCGTCACCCTT |
| | | GGACAGCCGGCCTCCATCTCCTGC |
| | | AAGTCCAGTCAGAGTCTGTTAGAC |
| | | AGTGGAAATCAAAAGAACTTCTTA |
| | | ACCTGGTATCAGCAGAAACCAGGG |
| | | AAAGCTCCTAAGCTCCTGATCTATT |
| | | GGGCATCCACTAGGGAATCTGGGG |
| | | TCCCCTCGAGGTTCAGTGGCAGTG |
| | | GATCTGGGACAGATTTCACCTTTAC |
| | | CATCAGTAGCCTGGAAGCTGAAGA |
| | | TGCTGCAACATATTACTGTCAGAAT |
| | | GATTATAGTTATCCGTACACGTTCG |
| | | GCCAAGGGACCAAGGTGGAAATCA |
| | | AACGTACGGTGGCTGCACCATCTG |
| | | TCTTCATCTTCCCGCCATCTGATGA |
| | | GCAGTTGAAATCTGGAACTGCCTCT |
| | | GTTGTGTGCCTGCTGAATAACTTCT |
| | | ATCCCAGAGAGGCCAAAGTACAGT |
| | | GGAAGGTGGATAACGCCCTCCAAT |
| | | CGGGTAACTCCCAGGAGAGTGTCA |
| | | CAGAGCAGGACAGCAAGGACAGC |
| | | ACCTACAGCCTCAGCAGCACCCTG |
| | | ACGCTGAGCAAAGCAGACTACGAG |
| | | AAACACAAAGTCTACGCCTGCGAA |
| | | GTCACCCATCAGGGCCTGAGCTCG |
| | | CCCGTCACAAAGAGCTTCAACAGG |
| | | GGAGAGTGT |
| BAP049-hum14 HC | | |
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 | HCDR1 | GYTFTTY |
| (Chothia) | | |
| SEQ ID NO: 141 | HCDR2 | YPGTGG |
| (Chothia) | | |
| SEQ ID NO: 139 | HCDR3 | WTTGTGAY |
| (Chothia) | | |
| SEQ ID NO: 216 | VH | QVQLVQSGAEVKKPGASVKV |
| | | SCKASGYTFTTYWMHWIRQSPSRGL |
| | | EWLGNIYPGTGGSNFDEKFKNRFTIS |
| | | RDNSKNTLYLQMNSLRAEDTAVYY |
| | | TRWTTGTGAYWGQGTTVTVSS |
| SEQ ID NO: 217 | DNA VH | CAGGTTCAGCTGGTGCAGTC |
| | | TGGAGCTGAGGTGAAGAAGCCTGG |
| | | GGCCTCAGTGAAGGTCTCCTGCAA |
| | | GGCTTCTGGCTACACATTCACCACT |
| | | TACTGGATGCACTGGATCAGGCAG |
| | | TCCCCATCGAGAGGCCTTGAGTGG |
| | | CTGGGTAATATTTATCCTGGTACTG |
| | | GTGGTTCTAACTTCGATGAGAAGTT |
| | | CAAGAACAGATTCACCATCTCCAG |
| | | AGACAATTCCAAGAACACGCTGTA |
| | | TCTTCAAATGAACAGCCTGAGAGC |
| | | CGAGGACACGGCCGTGTATTACTG |
| | | TACAAGATGGACTACTGGGACGGG |
| | | AGCTTACTGGGGCCAGGGCACCAC |
| | | CGTGACCGTGTCCTCC |
| SEQ ID NO: 218 | HC | QVQLVQSGAEVKKPGASVKV |
| | | SCKASGYTFTTYWMHWIRQSPSRGL |
| | | EWLGNIYPGTGGSNFDEKFKNRFTIS |
| | | RDNSKNTLYLQMNSLRAEDTAVYY |
| | | CTRWTTGTGAYWGQGTTVTVSSAS |
| | | TKGPSVFPLAPCSRSTSESTAALGCL |
| | | VKDYFPEPVTVSWNSGALTSGVHTF |
| | | PAVLQSSGLYSLSSVVTVPSSSLGTK |
| | | TYTCNVDHKPSNTKVDKRVESKYGP |
| | | PCPPCPAPEFLGGPSVFLFPPKPKDTL |
| | | MISRTPEVTCVVVDVSQEDPEVQFN |
| | | WYVDGVEVHNAKTKPREEQFNSTY |
| | | RVVSVLTVLHQDWLNGKEYKCKVS |
| | | NKGLPSSIEKTISKAKGQPREPQVYT |
| | | LPPSQEEMTKNQVSLTCLVKGFYPSD |
| | | IAVEWESNGQPENNYKTTPPVLDSD |
| | | GSFFLYSRLTVDKSRWQEGNVFSCS |
| | | VMHEALHNHYTQKSLSLSLGK |
| SEQ ID NO: 219 | DNA HC | CAGGTTCAGCTGGTGCAGTC |
| | | TGGAGCTGAGGTGAAGAAGCCTGG |
| | | GGCCTCAGTGAAGGTCTCCTGCAA |
| | | GGCTTCTGGCTACACATTCACCACT |
| | | TACTGGATGCACTGGATCAGGCAG |
| | | TCCCCATCGAGAGGCCTTGAGTGG |
| | | CTGGGTAATATTTATCCTGGTACTG |
| | | GTGGTTCTAACTTCGATGAGAAGTT |
| | | CAAGAACAGATTCACCATCTCCAG |
| | | AGACAATTCCAAGAACACGCTGTA |
| | | TCTTCAAATGAACAGCCTGAGAGC |
| | | CGAGGACACGGCCGTGTATTACTG |
| | | TACAAGATGGACTACTGGGACGGG |
| | | AGCTTACTGGGGCCAGGGCACCAC |
| | | CGTGACCGTGTCCTCCGCTTCCACC |
| | | AAGGGCCCATCCGTCTTCCCCCTGG |
| | | CGCCCTGCTCCAGGAGCACCTCCG |
| | | AGAGCACAGCCGCCCTGGGCTGCC |
| | | TGGTCAAGGACTACTTCCCCGAAC |
| | | CGGTGACGGTGTCGTGGAACTCAG |
| | | GCGCCCTGACCAGCGGCGTGCACA |
| | | CCTTCCCGGCTGTCCTACAGTCCTC |
| | | AGGACTCTACTCCCTCAGCAGCGT |
| | | GGTGACCGTGCCCTCCAGCAGCTT |
| | | GGGCACGAAGACCTACACCTGCAA |
| | | CGTAGATCACAAGCCCAGCAACAC |
| | | CAAGGTGGACAAGAGAGTTGAGTC |
| | | CAAATATGGTCCCCCATGCCCACC |
| | | GTGCCCAGCACCTGAGTTCCTGGG |
| | | GGGACCATCAGTCTTCCTGTTCCCC |
| | | CCAAAACCCAAGGACACTCTCATG |
| | | ATCTCCCGGACCCCTGAGGTCACGT |
| | | GCGTGGTGGTGGACGTGAGCCAGG |
| | | AAGACCCCGAGGTCCAGTTCAACT |
| | | GGTACGTGGATGGCGTGGAGGTGC |
| | | ATAATGCCAAGACAAAGCCGCGGG |
| | | AGGAGCAGTTCAACAGCACGTACC |
| | | GTGTGGTCAGCGTCCTCACCGTCCT |
| | | GCACCAGGACTGGCTGAACGGCAA |
| | | GGAGTACAAGTGCAAGGTGTCCAA |
| | | CAAAGGCCTCCCGTCCTCCATCGA |
| | | GAAAACCATCTCCAAAGCCAAAGG |
| | | GCAGCCCCGAGAGCCACAGGTGTA |
| | | CACCCTGCCCCCATCCCAGGAGGA |
| | | GATGACCAAGAACCAGGTCAGCCT |
| | | GACCTGCCTGGTCAAAGGCTTCTAC |
| | | CCCAGCGACATCGCCGTGGAGTGG |
| | | GAGAGCAATGGGCAGCCGGAGAAC |
| | | AACTACAAGACCACGCCTCCCGTG |
| | | CTGGACTCCGACGGCTCCTTCTTCC |
| | | TCTACAGCAGGCTAACCGTGGACA |
| | | AGAGCAGGTGGCAGGAGGGGAAT |
| | | GTCTTCTCATGCTCCGTGATGCATG |
| | | AGGCTCTGCACAACCACTACACAC |
| | | AGAAGAGCCTCTCCCTGTCTCTGGG |
| | | TAAA |
| BAP049-hum14 LC | | |
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 | LCDR1 | SQSLLDSGNQKNF |
| (Chothia) | | |
| SEQ ID NO: 150 | LCDR2 | WAS |
| (Chothia) | | |
| SEQ ID NO: 167 | LCDR3 | DYSYPY |
| (Chothia) | | |
| SEQ ID NO: 204 | VL | EIVLTQSPATLSLSPGERATLS |
| | | CKSSQSLLDSGNQKNFLTWYQQKPG |
| | | QAPRLLIYWASTRESGVPSRFSGSGS |
| | | GTDFTFTISSLEAEDAATYYCQNDYS |
| | | YPYTFGQGTKVEIK |
| SEQ ID NO: 205 | DNA VL | GAAATTGTGTTGACACAGTC |
| | | TCCAGCCACCCTGTCTTTGTCTCCA |
| | | GGGGAAAGAGCCACCCTCTCCTGC |
| | | AAGTCCAGTCAGAGTCTGTTAGAC |
| | | AGTGGAAATCAAAAGAACTTCTTG |
| | | ACCTGGTACCAGCAGAAACCTGGC |
| | | CAGGCTCCCAGGCTCCTCATCTATT |
| | | GGGCATCCACTAGGGAATCTGGGG |
| | | TCCCCTCGAGGTTCAGTGGCAGTG |
| | | GATCTGGGACAGATTTCACCTTTAC |
| | | CATCAGTAGCCTGGAAGCTGAAGA |
| | | TGCTGCAACATATTACTGTCAGAAT |
| | | GATTATAGTTATCCGTACACGTTCG |
| | | GCCAAGGGACCAAGGTGGAAATCA |
| | | AA |
| SEQ ID NO: 206 | LC | EIVLTQSPATLSLSPGERATLS |
| | | CKSSQSLLDSGNQKNFLTWYQQKPG |
| | | QAPRLLIYWASTRESGVPSRFSGSGS |
| | | GTDFTFTISSLEAEDAATYYCQNDYS |
| | | YPYTFGQGTKVEIKRTVAAPSVFIFPP |
| | | SDEQLKSGTASVVCLLNNFYPREAK |
| | | VQWKVDNALQSGNSQESVTEQDSK |
| | | DSTYSLSSTLTLSKADYEKHKVYAC |
| | | EVTHQGLSSPVTKSFNRGEC |
| SEQ ID NO: 207 | DNA LC | GAAATTGTGTTGACACAGTC |
| | | TCCAGCCACCCTGTCTTTGTCTCCA |
| | | GGGGAAAGAGCCACCCTCTCCTGC |
| | | AAGTCCAGTCAGAGTCTGTTAGAC |
| | | AGTGGAAATCAAAAGAACTTCTTG |
| | | ACCTGGTACCAGCAGAAACCTGGC |
| | | CAGGCTCCCAGGCTCCTCATCTATT |
| | | GGGCATCCACTAGGGAATCTGGGG |
| | | TCCCCTCGAGGTTCAGTGGCAGTG |
| | | GATCTGGGACAGATTTCACCTTTAC |
| | | CATCAGTAGCCTGGAAGCTGAAGA |
| | | TGCTGCAACATATTACTGTCAGAAT |
| | | GATTATAGTTATCCGTACACGTTCG |
| | | GCCAAGGGACCAAGGTGGAAATCA |
| | | AACGTACGGTGGCTGCACCATCTG |
| | | TCTTCATCTTCCCGCCATCTGATGA |
| | | GCAGTTGAAATCTGGAACTGCCTCT |
| | | GTTGTGTGCCTGCTGAATAACTTCT |
| | | ATCCCAGAGAGGCCAAAGTACAGT |
| | | GGAAGGTGGATAACGCCCTCCAAT |
| | | CGGGTAACTCCCAGGAGAGTGTCA |
| | | CAGAGCAGGACAGCAAGGACAGC |
| | | ACCTACAGCCTCAGCAGCACCCTG |
| | | ACGCTGAGCAAAGCAGACTACGAG |
| | | AAACACAAAGTCTACGCCTGCGAA |
| | | GTCACCCATCAGGGCCTGAGCTCG |
| | | CCCGTCACAAAGAGCTTCAACAGG |
| | | GGAGAGTGT |
| BAP049-hum15 HC | | |
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 | HCDR1 | GYTFTTY |
| (Chothia) | | |
| SEQ ID NO: 141 | HCDR2 | YPGTGG |
| (Chothia) | | |
| SEQ ID NO: 139 | HCDR3 | WTTGTGAY |
| (Chothia) | | |
| SEQ ID NO: 216 | VH | QVQLVQSGAEVKKPGASVKV |
| | | SCKASGYTFTTYWMHWIRQSPSRGL |
| | | EWLGNIYPGTGGSNFDEKFKNRFTIS |
| | | RDNSKNTLYLQMNSLRAEDTAVYY |
| | | CTRWTTGTGAYWGQGTTVTVSS |
| SEQ ID NO: 217 | DNA VH | CAGGTTCAGCTGGTGCAGTC |
| | | TGGAGCTGAGGTGAAGAAGCCTGG |
| | | GGCCTCAGTGAAGGTCTCCTGCAA |
| | | GGCTTCTGGCTACACATTCACCACT |
| | | TACTGGATGCACTGGATCAGGCAG |
| | | TCCCCATCGAGAGGCCTTGAGTGG |
| | | CTGGGTAATATTTATCCTGGTACTG |
| | | GTGGTTCTAACTTCGATGAGAAGTT |
| | | CAAGAACAGATTCACCATCTCCAG |
| | | AGACAATTCCAAGAACACGCTGTA |
| | | TCTTCAAATGAACAGCCTGAGAGC |
| | | CGAGGACACGGCCGTGTATTACTG |
| | | TACAAGATGGACTACTGGGACGGG |
| | | AGCTTACTGGGGCCAGGGCACCAC |
| | | CGTGACCGTGTCCTCC |
| SEQ ID NO: 218 | HC | QVQLVQSGAEVKKPGASVKV |
| | | SCKASGYTFTTYWMHWIRQSPSRGL |
| | | EWLGNIYPGTGGSNFDEKFKNRFTIS |
| | | RDNSKNTLYLQMNSLRAEDTAVYY |
| | | CTRWTTGTGAYWGQGTTVTVSSAS |
| | | TKGPSVFPLAPCSRSTSESTAALGCL |
| | | VKDYFPEPVTVSWNSGALTSGVHTF |
| | | PAVLQSSGLYSLSSVVTVPSSSLGTK |
| | | TYTCNVDHKPSNTKVDKRVESKYGP |
| | | PCPPCPAPEFLGGPSVFLFPPKPKDTL |
| | | MISRTPEVTCVVVDVSQEDPEVQFN |
| | | WYVDGVEVHNAKTKPREEQFNSTY |
| | | RVVSVLTVLHQDWLNGKEYKCKVS |
| | | NKGLPSSIEKTISKAKGQPREPQVYT |
| | | LPPSQEEMTKNQVSLTCLVKGFYPSD |
| | | IAVEWESNGQPENNYKTTPPVLDSD |
| | | GSFFLYSRLTVDKSRWQEGNVFSCS |
| | | VMHEALHNHYTQKSLSLSLGK |
| SEQ ID NO: 219 | DNA HC | CAGGTTCAGCTGGTGCAGTC |
| | | TGGAGCTGAGGTGAAGAAGCCTGG |
| | | GGCCTCAGTGAAGGTCTCCTGCAA |
| | | GGCTTCTGGCTACACATTCACCACT |
| | | TACTGGATGCACTGGATCAGGCAG |
| | | TCCCCATCGAGAGGCCTTGAGTGG |
| | | CTGGGTAATATTTATCCTGGTACTG |
| | | GTGGTTCTAACTTCGATGAGAAGTT |
| | | CAAGAACAGATTCACCATCTCCAG |
| | | AGACAATTCCAAGAACACGCTGTA |
| | | TCTTCAAATGAACAGCCTGAGAGC |
| | | CGAGGACACGGCCGTGTATTACTG |
| | | TACAAGATGGACTACTGGGACGGG |
| | | AGCTTACTGGGGCCAGGGCACCAC |
| | | CGTGACCGTGTCCTCCGCTTCCACC |
| | | AAGGGCCCATCCGTCTTCCCCCTGG |
| | | CGCCCTGCTCCAGGAGCACCTCCG |
| | | AGAGCACAGCCGCCCTGGGCTGCC |
| | | TGGTCAAGGACTACTTCCCCGAAC |
| | | CGGTGACGGTGTCGTGGAACTCAG |
| | | GCGCCCTGACCAGCGGCGTGCACA |
| | | CCTTCCCGGCTGTCCTACAGTCCTC |
| | | AGGACTCTACTCCCTCAGCAGCGT |
| | | GGTGACCGTGCCCTCCAGCAGCTT |
| | | GGGCACGAAGACCTACACCTGCAA |
| | | CGTAGATCACAAGCCCAGCAACAC |
| | | CAAGGTGGACAAGAGAGTTGAGTC |
| | | CAAATATGGTCCCCCATGCCCACC |
| | | GTGCCCAGCACCTGAGTTCCTGGG |
| | | GGGACCATCAGTCTTCCTGTTCCCC |
| | | CCAAAACCCAAGGACACTCTCATG |
| | | ATCTCCCGGACCCCTGAGGTCACGT |
| | | GCGTGGTGGTGGACGTGAGCCAGG |
| | | AAGACCCCGAGGTCCAGTTCAACT |
| | | GGTACGTGGATGGCGTGGAGGTGC |
| | | ATAATGCCAAGACAAAGCCGCGGG |
| | | AGGAGCAGTTCAACAGCACGTACC |
| | | GTGTGGTCAGCGTCCTCACCGTCCT |
| | | GCACCAGGACTGGCTGAACGGCAA |
| | | GGAGTACAAGTGCAAGGTGTCCAA |
| | | CAAAGGCCTCCCGTCCTCCATCGA |
| | | GAAAACCATCTCCAAAGCCAAAGG |
| | | GCAGCCCCGAGAGCCACAGGTGTA |
| | | CACCCTGCCCCCATCCCAGGAGGA |
| | | GATGACCAAGAACCAGGTCAGCCT |
| | | GACCTGCCTGGTCAAAGGCTTCTAC |
| | | CCCAGCGACATCGCCGTGGAGTGG |
| | | GAGAGCAATGGGCAGCCGGAGAAC |
| | | AACTACAAGACCACGCCTCCCGTG |
| | | CTGGACTCCGACGGCTCCTTCTTCC |
| | | TCTACAGCAGGCTAACCGTGGACA |
| | | AGAGCAGGTGGCAGGAGGGGAAT |
| | | GTCTTCTCATGCTCCGTGATGCATG |
| | | AGGCTCTGCACAACCACTACACAC |
| | | AGAAGAGCCTCTCCCTGTCTCTGGG |
| | | TAAA |
| BAP049-hum15 LC | | |
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 | LCDR1 | SQSLLDSGNQKNF |
| (Chothia) | | |
| SEQ ID NO: 150 | LCDR2 | WAS |
| (Chothia) | | |
| SEQ ID NO: 167 | LCDR3 | DYSYPY |
| (Chothia) | | |
| SEQ ID NO: 200 | VL | EIVLTQSPDFQSVTPKEKVTIT |
| | | CKSSQSLLDSGNQKNFLTWYQQKPG |
| | | QAPRLLIYWASTRESGVPSRFSGSGS |
| | | GTDFTFTISSLEAEDAATYYCQNDYS |
| | | YPYTFGQGTKVEIK |
| SEQ ID NO: 201 | DNA VL | GAAATTGTGCTGACTCAGTC |
| | | TCCAGACTTTCAGTCTGTGACTCCA |
| | | AAGGAGAAAGTCACCATCACCTGC |
| | | AAGTCCAGTCAGAGTCTGTTAGAC |
| | | AGTGGAAATCAAAAGAACTTCTTG |
| | | ACCTGGTACCAGCAGAAACCTGGC |
| | | CAGGCTCCCAGGCTCCTCATCTATT |
| | | GGGCATCCACTAGGGAATCTGGGG |
| | | TCCCCTCGAGGTTCAGTGGCAGTG |
| | | GATCTGGGACAGATTTCACCTTTAC |
| | | CATCAGTAGCCTGGAAGCTGAAGA |
| | | TGCTGCAACATATTACTGTCAGAAT |
| | | GATTATAGTTATCCGTACACGTTCG |
| | | GCCAAGGGACCAAGGTGGAAATCA |
| | | AA |
| SEQ ID NO: 202 | LC | EIVLTQSPDFQSVTPKEKVTIT |
| | | CKSSQSLLDSGNQKNFLTWYQQKPG |
| | | QAPRLLIYWASTRESGVPSRFSGSGS |
| | | GTDFTFTISSLEAEDAATYYCQNDYS |
| | | YPYTFGQGTKVEIKRTVAAPSVFIFPP |
| | | SDEQLKSGTASVVCLLNNFYPREAK |
| | | VQWKVDNALQSGNSQESVTEQDSK |
| | | DSTYSLSSTLTLSKADYEKHKVYAC |
| | | EVTHQGLSSPVTKSFNRGEC |
| SEQ ID NO: 203 | DNA LC | GAAATTGTGCTGACTCAGTC |
| | | TCCAGACTTTCAGTCTGTGACTCCA |
| | | AAGGAGAAAGTCACCATCACCTGC |
| | | AAGTCCAGTCAGAGTCTGTTAGAC |
| | | AGTGGAAATCAAAAGAACTTCTTG |
| | | ACCTGGTACCAGCAGAAACCTGGC |
| | | CAGGCTCCCAGGCTCCTCATCTATT |
| | | GGGCATCCACTAGGGAATCTGGGG |
| | | TCCCCTCGAGGTTCAGTGGCAGTG |
| | | GATCTGGGACAGATTTCACCTTTAC |
| | | CATCAGTAGCCTGGAAGCTGAAGA |
| | | TGCTGCAACATATTACTGTCAGAAT |
| | | GATTATAGTTATCCGTACACGTTCG |
| | | GCCAAGGGACCAAGGTGGAAATCA |
| | | AACGTACGGTGGCTGCACCATCTG |
| | | TCTTCATCTTCCCGCCATCTGATGA |
| | | GCAGTTGAAATCTGGAACTGCCTCT |
| | | GTTGTGTGCCTGCTGAATAACTTCT |
| | | ATCCCAGAGAGGCCAAAGTACAGT |
| | | GGAAGGTGGATAACGCCCTCCAAT |
| | | CGGGTAACTCCCAGGAGAGTGTCA |
| | | CAGAGCAGGACAGCAAGGACAGC |
| | | ACCTACAGCCTCAGCAGCACCCTG |
| | | ACGCTGAGCAAAGCAGACTACGAG |
| | | AAACACAAAGTCTACGCCTGCGAA |
| | | GTCACCCATCAGGGCCTGAGCTCG |
| | | CCCGTCACAAAGAGCTTCAACAGG |
| | | GGAGAGTGT |
| BAP049-hum16 HC | | |
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 | HCDR1 | GYTFTTY |
| (Chothia) | | |
| SEQ ID NO: 141 | HCDR2 | YPGTGG |
| (Chothia) | | |
| SEQ ID NO: 139 | HCDR3 | WTTGTGAY |
| (Chothia) | | |
| SEQ ID NO: 220 | VH | EVQLVQSGAEVKKPGESLRIS |
| | | CKGSGYTFTTYWMHWVRQAPGQGL |
| | | EWMGNIYPGTGGSNFDEKFKNRFTIS |
| | | RDNSKNTLYLQMNSLRAEDTAVYY |
| | | CTRWTTGTGAYWGQGTTVTVSS |
| SEQ ID NO: 221 | DNA VH | GAAGTGCAGCTGGTGCAGTC |
| | | TGGAGCAGAGGTGAAAAAGCCCGG |
| | | GGAGTCTCTGAGGATCTCCTGTAA |
| | | GGGTTCTGGCTACACATTCACCACT |
| | | TACTGGATGCACTGGGTGCGACAG |
| | | GCCCCTGGACAAGGGCTTGAGTGG |
| | | ATGGGTAATATTTATCCTGGTACTG |
| | | GTGGTTCTAACTTCGATGAGAAGTT |
| | | CAAGAACAGATTCACCATCTCCAG |
| | | AGACAATTCCAAGAACACGCTGTA |
| | | TCTTCAAATGAACAGCCTGAGAGC |
| | | CGAGGACACGGCCGTGTATTACTG |
| | | TACAAGATGGACTACTGGGACGGG |
| | | AGCTTATTGGGGCCAGGGCACCAC |
| | | CGTGACCGTGTCCTCC |
| SEQ ID NO: 222 | HC | EVQLVQSGAEVKKPGESLRIS |
| | | CKGSGYTFTTYWMHWVRQAPGQGL |
| | | EWMGNIYPGTGGSNFDEKFKNRFTIS |
| | | RDNSKNTLYLQMNSLRAEDTAVYY |
| | | CTRWTTGTGAYWGQGTTVTVSSAS |
| | | TKGPSVFPLAPCSRSTSESTAALGCL |
| | | VKDYFPEPVTVSWNSGALTSGVHTF |
| | | PAVLQSSGLYSLSSVVTVPSSSLGTK |
| | | TYTCNVDHKPSNTKVDKRVESKYGP |
| | | PCPPCPAPEFLGGPSVFLFPPKPKDTL |
| | | MISRTPEVTCVVVDVSQEDPEVQFN |
| | | WYVDGVEVHNAKTKPREEQFNSTY |
| | | RVVSVLTVLHQDWLNGKEYKCKVS |
| | | NKGLPSSIEKTISKAKGQPREPQVYT |
| | | LPPSQEEMTKNQVSLTCLVKGFYPSD |
| | | IAVEWESNGQPENNYKTTPPVLDSD |
| | | GSFFLYSRLTVDKSRWQEGNVFSCS |
| | | VMHEALHNHYTQKSLSLSLGK |
| SEQ ID NO: 223 | DNA HC | GAAGTGCAGCTGGTGCAGTC |
| | | TGGAGCAGAGGTGAAAAAGCCCGG |
| | | GGAGTCTCTGAGGATCTCCTGTAA |
| | | GGGTTCTGGCTACACATTCACCACT |
| | | TACTGGATGCACTGGGTGCGACAG |
| | | GCCCCTGGACAAGGGCTTGAGTGG |
| | | ATGGGTAATATTTATCCTGGTACTG |
| | | GTGGTTCTAACTTCGATGAGAAGTT |
| | | CAAGAACAGATTCACCATCTCCAG |
| | | AGACAATTCCAAGAACACGCTGTA |
| | | TCTTCAAATGAACAGCCTGAGAGC |
| | | CGAGGACACGGCCGTGTATTACTG |
| | | TACAAGATGGACTACTGGGACGGG |
| | | AGCTTATTGGGGCCAGGGCACCAC |
| | | CGTGACCGTGTCCTCCGCTTCCACC |
| | | AAGGGCCCATCCGTCTTCCCCCTGG |
| | | CGCCCTGCTCCAGGAGCACCTCCG |
| | | AGAGCACAGCCGCCCTGGGCTGCC |
| | | TGGTCAAGGACTACTTCCCCGAAC |
| | | CGGTGACGGTGTCGTGGAACTCAG |
| | | GCGCCCTGACCAGCGGCGTGCACA |
| | | CCTTCCCGGCTGTCCTACAGTCCTC |
| | | AGGACTCTACTCCCTCAGCAGCGT |
| | | GGTGACCGTGCCCTCCAGCAGCTT |
| | | GGGCACGAAGACCTACACCTGCAA |
| | | CGTAGATCACAAGCCCAGCAACAC |
| | | CAAGGTGGACAAGAGAGTTGAGTC |
| | | CAAATATGGTCCCCCATGCCCACC |
| | | GTGCCCAGCACCTGAGTTCCTGGG |
| | | GGGACCATCAGTCTTCCTGTTCCCC |
| | | CCAAAACCCAAGGACACTCTCATG |
| | | ATCTCCCGGACCCCTGAGGTCACGT |
| | | GCGTGGTGGTGGACGTGAGCCAGG |
| | | AAGACCCCGAGGTCCAGTTCAACT |
| | | GGTACGTGGATGGCGTGGAGGTGC |
| | | ATAATGCCAAGACAAAGCCGCGGG |
| | | AGGAGCAGTTCAACAGCACGTACC |
| | | GTGTGGTCAGCGTCCTCACCGTCCT |
| | | GCACCAGGACTGGCTGAACGGCAA |
| | | GGAGTACAAGTGCAAGGTGTCCAA |
| | | CAAAGGCCTCCCGTCCTCCATCGA |
| | | GAAAACCATCTCCAAAGCCAAAGG |
| | | GCAGCCCCGAGAGCCACAGGTGTA |
| | | CACCCTGCCCCCATCCCAGGAGGA |
| | | GATGACCAAGAACCAGGTCAGCCT |
| | | GACCTGCCTGGTCAAAGGCTTCTAC |
| | | CCCAGCGACATCGCCGTGGAGTGG |
| | | GAGAGCAATGGGCAGCCGGAGAAC |
| | | AACTACAAGACCACGCCTCCCGTG |
| | | CTGGACTCCGACGGCTCCTTCTTCC |
| | | TCTACAGCAGGCTAACCGTGGACA |
| | | AGAGCAGGTGGCAGGAGGGGAAT |
| | | GTCTTCTCATGCTCCGTGATGCATG |
| | | AGGCTCTGCACAACCACTACACAC |
| | | AGAAGAGCCTCTCCCTGTCTCTGGG |
| | | TAAA |
| BAP049-hum16 LC | | |
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 | LCDR1 | SQSLLDSGNQKNF |
| (Chothia) | | |
| SEQ ID NO: 150 | LCDR2 | WAS |
| (Chothia) | | |
| SEQ ID NO: 167 | LCDR3 | DYSYPY |
| (Chothia) | | |
| SEQ ID NO: 200 | VL | EIVLTQSPDFQSVTPKEKVTIT |
| | | CKSSQSLLDSGNQKNFLTWYQQKPG |
| | | QAPRLLIYWASTRESGVPSRFSGSGS |
| | | GTDFTFTISSLEAEDAATYYCQNDYS |
| | | YPYTFGQGTKVEIK |
| SEQ ID NO: 201 | DNA VL | GAAATTGTGCTGACTCAGTC |
| | | TCCAGACTTTCAGTCTGTGACTCCA |
| | | AAGGAGAAAGTCACCATCACCTGC |
| | | AAGTCCAGTCAGAGTCTGTTAGAC |
| | | AGTGGAAATCAAAAGAACTTCTTG |
| | | ACCTGGTACCAGCAGAAACCTGGC |
| | | CAGGCTCCCAGGCTCCTCATCTATT |
| | | GGGCATCCACTAGGGAATCTGGGG |
| | | TCCCCTCGAGGTTCAGTGGCAGTG |
| | | GATCTGGGACAGATTTCACCTTTAC |
| | | CATCAGTAGCCTGGAAGCTGAAGA |
| | | TGCTGCAACATATTACTGTCAGAAT |
| | | GATTATAGTTATCCGTACACGTTCG |
| | | GCCAAGGGACCAAGGTGGAAATCA |
| | | AA |
| SEQ ID NO: 202 | LC | EIVLTQSPDFQSVTPKEKVTIT |
| | | CKSSQSLLDSGNQKNFLTWYQQKPG |
| | | QAPRLLIYWASTRESGVPSRFSGSGS |
| | | GTDFTFTISSLEAEDAATYYCQNDYS |
| | | YPYTFGQGTKVEIKRTVAAPSVFIFPP |
| | | SDEQLKSGTASVVCLLNNFYPREAK |
| | | VQWKVDNALQSGNSQESVTEQDSK |
| | | DSTYSLSSTLTLSKADYEKHKVYAC |
| | | EVTHQGLSSPVTKSFNRGEC |
| SEQ ID NO: 203 | DNA LC | GAAATTGTGCTGACTCAGTC |
| | | TCCAGACTTTCAGTCTGTGACTCCA |
| | | AAGGAGAAAGTCACCATCACCTGC |
| | | AAGTCCAGTCAGAGTCTGTTAGAC |
| | | AGTGGAAATCAAAAGAACTTCTTG |
| | | ACCTGGTACCAGCAGAAACCTGGC |
| | | CAGGCTCCCAGGCTCCTCATCTATT |
| | | GGGCATCCACTAGGGAATCTGGGG |
| | | TCCCCTCGAGGTTCAGTGGCAGTG |
| | | GATCTGGGACAGATTTCACCTTTAC |
| | | CATCAGTAGCCTGGAAGCTGAAGA |
| | | TGCTGCAACATATTACTGTCAGAAT |
| | | GATTATAGTTATCCGTACACGTTCG |
| | | GCCAAGGGACCAAGGTGGAAATCA |
| | | AACGTACGGTGGCTGCACCATCTG |
| | | TCTTCATCTTCCCGCCATCTGATGA |
| | | GCAGTTGAAATCTGGAACTGCCTCT |
| | | GTTGTGTGCCTGCTGAATAACTTCT |
| | | ATCCCAGAGAGGCCAAAGTACAGT |
| | | GGAAGGTGGATAACGCCCTCCAAT |
| | | CGGGTAACTCCCAGGAGAGTGTCA |
| | | CAGAGCAGGACAGCAAGGACAGC |
| | | ACCTACAGCCTCAGCAGCACCCTG |
| | | ACGCTGAGCAAAGCAGACTACGAG |
| | | AAACACAAAGTCTACGCCTGCGAA |
| | | GTCACCCATCAGGGCCTGAGCTCG |
| | | CCCGTCACAAAGAGCTTCAACAGG |
| | | GGAGAGTGT |
| BAP049-Clone-A HC | | |
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 | HCDR1 | GYTFTTY |
| (Chothia) | | |
| SEQ ID NO: 141 | HCDR2 | YPGTGG |
| (Chothia) | | |
| SEQ ID NO: 139 | HCDR3 | WTTGTGAY |
| (Chothia) | | |
| SEQ ID NO: 172 | VH | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSS |
| SEQ ID NO: 224 | DNA VH | GAAGTGCAGCTGGTGCAGTC |
| | | TGGCGCCGAAGTGAAGAAGCCTGG |
| | | CGAGTCCCTGCGGATCTCCTGCAA |
| | | GGGCTCTGGCTACACCTTCACCACC |
| | | TACTGGATGCACTGGGTGCGACAG |
| | | GCTACCGGCCAGGGCCTGGAATGG |
| | | ATGGGCAACATCTATCCTGGCACC |
| | | GGCGGCTCCAACTTCGACGAGAAG |
| | | TTCAAGAACAGAGTGACCATCACC |
| | | GCCGACAAGTCCACCTCCACCGCC |
| | | TACATGGAACTGTCCTCCCTGAGAT |
| | | CCGAGGACACCGCCGTGTACTACT |
| | | GCACCCGGTGGACAACCGGCACAG |
| | | GCGCTTATTGGGGCCAGGGCACCA |
| | | CAGTGACCGTGTCCTCT |
| SEQ ID NO: 225 | HC | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSSAS |
| | | TKGPSVFPLAPCSRSTSESTAALGCL |
| | | VKDYFPEPVTVSWNSGALTSGVHTF |
| | | PAVLQSSGLYSLSSVVTVPSSSLGTK |
| | | TYTCNVDHKPSNTKVDKRVESKYGP |
| | | PCPPCPAPEFLGGPSVFLFPPKPKDTL |
| | | MISRTPEVTCVVVDVSQEDPEVQFN |
| | | WYVDGVEVHNAKTKPREEQFNSTY |
| | | RVVSVLTVLHQDWLNGKEYKCKVS |
| | | NKGLPSSIEKTISKAKGQPREPQVYT |
| | | LPPSQEEMTKNQVSLTCLVKGFYPSD |
| | | IAVEWESNGQPENNYKTTPPVLDSD |
| | | GSFFLYSRLTVDKSRWQEGNVFSCS |
| | | VMHEALHNHYTQKSLSLSLG |
| SEQ ID NO: 226 | DNA HC | GAAGTGCAGCTGGTGCAGTC |
| | | TGGCGCCGAAGTGAAGAAGCCTGG |
| | | CGAGTCCCTGCGGATCTCCTGCAA |
| | | GGGCTCTGGCTACACCTTCACCACC |
| | | TACTGGATGCACTGGGTGCGACAG |
| | | GCTACCGGCCAGGGCCTGGAATGG |
| | | ATGGGCAACATCTATCCTGGCACC |
| | | GGCGGCTCCAACTTCGACGAGAAG |
| | | TTCAAGAACAGAGTGACCATCACC |
| | | GCCGACAAGTCCACCTCCACCGCC |
| | | TACATGGAACTGTCCTCCCTGAGAT |
| | | CCGAGGACACCGCCGTGTACTACT |
| | | GCACCCGGTGGACAACCGGCACAG |
| | | GCGCTTATTGGGGCCAGGGCACCA |
| | | CAGTGACCGTGTCCTCTGCTTCTAC |
| | | CAAGGGGCCCAGCGTGTTCCCCCT |
| | | GGCCCCCTGCTCCAGAAGCACCAG |
| | | CGAGAGCACAGCCGCCCTGGGCTG |
| | | CCTGGTGAAGGACTACTTCCCCGA |
| | | GCCCGTGACCGTGTCCTGGAACAG |
| | | CACCTTCCCCGCCGTGCTGCAGAGC |
| | | AGCGGCCTGTACAGCCTGAGCAGC |
| | | GTGGTGACCGTGCCCAGCAGCAGC |
| | | CTGGGCACCAAGACCTACACCTGT |
| | | AACGTGGACCACAAGCCCAGCAAC |
| | | ACCAAGGTGGACAAGAGGGTGGAG |
| | | AGCAAGTACGGCCCACCCTGCCCC |
| | | CCCTGCCCAGCCCCCGAGTTCCTGG |
| | | GCGGACCCAGCGTGTTCCTGTTCCC |
| | | CCCCAAGCCCAAGGACACCCTGAT |
| | | GATCAGCAGAACCCCCGAGGTGAC |
| | | CTGTGTGGTGGTGGACGTGTCCCA |
| | | GGAGGACCCCGAGGTCCAGTTCAA |
| | | CTGGTACGTGGACGGCGTGGAGGT |
| | | GCACAACGCCAAGACCAAGCCCAG |
| | | AGAGGAGCAGTTTAACAGCACCTA |
| | | CCGGGTGGTGTCCGTGCTGACCGT |
| | | GCTGCACCAGGACTGGCTGAACGG |
| | | CAAAGAGTACAAGTGTAAGGTCTC |
| | | CAACAAGGGCCTGCCAAGCAGCAT |
| | | CGAAAAGACCATCAGCAAGGCCAA |
| | | GGGCCAGCCTAGAGAGCCCCAGGT |
| | | CTACACCCTGCCACCCAGCCAAGA |
| | | GGAGATGACCAAGAACCAGGTGTC |
| | | CCTGACCTGTCTGGTGAAGGGCTTC |
| | | TACCCAAGCGACATCGCCGTGGAG |
| | | TGGGAGAGCAACGGCCAGCCCGAG |
| | | AACAACTACAAGACCACCCCCCCA |
| | | GTGCTGGACAGCGACGGCAGCTTC |
| | | TTCCTGTACAGCAGGCTGACCGTG |
| | | GACAAGTCCAGATGGCAGGAGGGC |
| | | AACGTCTTTAGCTGCTCCGTGATGC |
| | | ACGAGGCCCTGCACAACCACTACA |
| | | CCCAGAAGAGCCTGAGCCTGTCCC |
| | | TGGGC |
| BAP049-Clone-A LC | | |
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 | LCDR1 | SQSLLDSGNQKNF |
| (Chothia) | | |
| SEQ ID NO: 150 | LCDR2 | WAS |
| (Chothia) | | |
| SEQ ID NO: 167 | LCDR3 | DYSYPY |
| (Chothia) | | |
| SEQ ID NO: 176 | VL | EIVLTQSPATLSLSPGERATLS |
| | | CKSSQSLLDSGNQKNFLTWYQQKPG |
| | | QAPRLLIYWASTRESGVPSRFSGSGS |
| | | GTEFTLTISSLQPDDFATYYCQNDYS |
| | | YPYTFGQGTKVEIK |
| SEQ ID NO: 227 | DNA VL | GAGATCGTGCTGACCCAGTC |
| | | CCCTGCCACCCTGTCACTGTCTCCA |
| | | GGCGAGAGAGCTACCCTGTCCTGC |
| | | AAGTCCTCCCAGTCCCTGCTGGACT |
| | | CCGGCAACCAGAAGAACTTCCTGA |
| | | CCTGGTATCAGCAGAAGCCCGGCC |
| | | AGGCCCCCAGACTGCTGATCTACT |
| | | GGGCCTCCACCCGGGAATCTGGCG |
| | | TGCCCTCTAGATTCTCCGGCTCCGG |
| | | CTCTGGCACCGAGTTTACCCTGACC |
| | | ATCTCCAGCCTGCAGCCCGACGAC |
| | | TTCGCCACCTACTACTGCCAGAACG |
| | | ACTACTCCTACCCCTACACCTTCGG |
| | | CCAGGGCACCAAGGTGGAAATCAA |
| | | G |
| SEQ ID NO: 178 | LC | EIVLTQSPATLSLSPGERATLS |
| | | CKSSQSLLDSGNQKNFLTWYQQKPG |
| | | QAPRLLIYWASTRESGVPSRFSGSGS |
| | | GTEFTLTISSLQPDDFATYYCQNDYS |
| | | YPYTFGQGTKVEIKRTVAAPSVFIFPP |
| | | SDEQLKSGTASVVCLLNNFYPREAK |
| | | VQWKVDNALQSGNSQESVTEQDSK |
| | | DSTYSLSSTLTLSKADYEKHKVYAC |
| | | EVTHQGLSSPVTKSFNRGEC |
| SEQ ID NO: 228 | DNA LC | GAGATCGTGCTGACCCAGTC |
| | | CCCTGCCACCCTGTCACTGTCTCCA |
| | | GGCGAGAGAGCTACCCTGTCCTGC |
| | | AAGTCCTCCCAGTCCCTGCTGGACT |
| | | CCGGCAACCAGAAGAACTTCCTGA |
| | | CCTGGTATCAGCAGAAGCCCGGCC |
| | | AGGCCCCCAGACTGCTGATCTACT |
| | | GGGCCTCCACCCGGGAATCTGGCG |
| | | TGCCCTCTAGATTCTCCGGCTCCGG |
| | | CTCTGGCACCGAGTTTACCCTGACC |
| | | ATCTCCAGCCTGCAGCCCGACGAC |
| | | TTCGCCACCTACTACTGCCAGAACG |
| | | ACTACTCCTACCCCTACACCTTCGG |
| | | CCAGGGCACCAAGGTGGAAATCAA |
| | | GCGTACGGTGGCCGCTCCCAGCGT |
| | | GTTCATCTTCCCCCCAAGCGACGAG |
| | | CAGCTGAAGAGCGGCACCGCCAGC |
| | | GTGGTGTGTCTGCTGAACAACTTCT |
| | | ACCCCAGGGAGGCCAAGGTGCAGT |
| | | GGAAGGTGGACAACGCCCTGCAGA |
| | | GCGGCAACAGCCAGGAGAGCGTCA |
| | | CCGAGCAGGACAGCAAGGACTCCA |
| | | CCTACAGCCTGAGCAGCACCCTGA |
| | | CCCTGAGCAAGGCCGACTACGAGA |
| | | AGCACAAGGTGTACGCCTGTGAGG |
| | | TGACCCACCAGGGCCTGTCCAGCC |
| | | CCGTGACCAAGAGCTTCAACAGGG |
| | | GCGAGTGC |
| BAP049-Clone-B HC | | |
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 | HCDR1 | GYTFTTY |
| (Chothia) | | |
| SEQ ID NO: 141 | HCDR2 | YPGTGG |
| (Chothia) | | |
| SEQ ID NO: 139 | HCDR3 | WTTGTGAY |
| (Chothia) | | |
| SEQ ID NO: 172 | VH | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSS |
| SEQ ID NO: 229 | DNA VH | Gaggtgcagctggtgcagtcaggcgccgaa |
| | | gtgaagaagcccggcgagtcactgagaattagctgtaaa |
| | | ggttcaggctacaccttcactacctactggatgcactgggt |
| | | ccgccaggctaccggtcaaggcctcgagtggatgggta |
| | | atatctaccccggcaccggcggctctaacttcgacgaga |
| | | agtttaagaatagagtgactatcaccgccgataagtctact |
| | | agcaccgcctatatggaactgtctagcctgagatcagag |
| | | gacaccgccgtctactactgcactaggtggactaccggc |
| | | acaggcgcctactggggtcaaggcactaccgtgaccgt |
| | | gtctagc |
| SEQ ID NO: 225 | HC | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSSAS |
| | | TKGPSVFPLAPCSRSTSESTAALGCL |
| | | VKDYFPEPVTVSWNSGALTSGVHTF |
| | | PAVLQSSGLYSLSSVVTVPSSSLGTK |
| | | TYTCNVDHKPSNTKVDKRVESKYGP |
| | | PCPPCPAPEFLGGPSVFLFPPKPKDTL |
| | | MISRTPEVTCVVVDVSQEDPEVQFN |
| | | WYVDGVEVHNAKTKPREEQFNSTY |
| | | RVVSVLTVLHQDWLNGKEYKCKVS |
| | | NKGLPSSIEKTISKAKGQPREPQVYT |
| | | LPPSQEEMTKNQVSLTCLVKGFYPSD |
| | | IAVEWESNGQPENNYKTTPPVLDSD |
| | | GSFFLYSRLTVDKSRWQEGNVFSCS |
| | | VMHEALHNHYTQKSLSLSLG |
| SEQ ID NO: 230 | DNA HC | gaggtgcagctggtgcagtcaggcgccgaag |
| | | tgaagaagcccggcgagtcactgagaattagctgtaaag |
| | | gttcaggctacaccttcactacctactggatgcactgggtc |
| | | cgccaggctaccggtcaaggcctcgagtggatgggtaat |
| | | atctaccccggcaccggcggctctaacttcgacgagaag |
| | | tttaagaatagagtgactatcaccgccgataagtctactag |
| | | caccgcctatatggaactgtctagcctgagatcagagga |
| | | caccgccgtctactactgcactaggtggactaccggcac |
| | | aggcgcctactggggtcaaggcactaccgtgaccgtgtc |
| | | tagcgctagcactaagggcccgtccgtgttccccctggc |
| | | accttgtagccggagcactagcgaatccaccgctgccct |
| | | cggctgcctggtcaaggattacttcccggagcccgtgac |
| | | cgtgtcctggaacagcggagccctgacctccggagtgc |
| | | acaccttccccgctgtgctgcagagctccgggctgtactc |
| | | gctgtcgtcggtggtcacggtgccttcatctagcctgggt |
| | | accaagacctacacttgcaacgtggaccacaagccttcc |
| | | aacactaaggtggacaagcgcgtcgaatcgaagtacgg |
| | | cccaccgtgcccgccttgtcccgcgccggagttcctcgg |
| | | cggtccctcggtctttctgttcccaccgaagcccaaggac |
| | | actttgatgatttcccgcacccctgaagtgacatgcgtggt |
| | | cgtggacgtgtcacaggaagatccggaggtgcagttcaa |
| | | ttggtacgtggatggcgtcgaggtgcacaacgccaaaac |
| | | caagccgagggaggagcagttcaactccacttaccgcgt |
| | | cgtgtccgtgctgacggtgctgcatcaggactggctgaa |
| | | cgggaaggagtacaagtgcaaagtgtccaacaagggac |
| | | ttcctagctcaatcgaaaagaccatctcgaaagccaagg |
| | | gacagccccgggaaccccaagtgtataccctgccaccg |
| | | agccaggaagaaatgactaagaaccaagtctcattgactt |
| | | gccttgtgaagggcttctacccatcggatatcgccgtgga |
| | | atgggagtccaacggccagccggaaaacaactacaaga |
| | | ccacccctccggtgctggactcagacggatccttcttcct |
| | | ctactcgcggctgaccgtggataagagcagatggcagg |
| | | agggaaatgtgttcagctgttctgtgatgcatgaagccctg |
| | | cacaaccactacactcagaagtccctgtccctctccctgg |
| | | ga |
| BAP049-Clone-B LC | | |
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 | LCDR1 | SQSLLDSGNQKNF |
| (Chothia) | | |
| SEQ ID NO: 150 | LCDR2 | WAS |
| (Chothia) | | |
| SEQ ID NO: 167 | LCDR3 | DYSYPY |
| (Chothia) | | |
| SEQ ID NO: 188 | VL | EIVLTQSPATLSLSPGERATLS |
| | | CKSSQSLLDSGNQKNFLTWYQQKPG |
| | | KAPKLLIYWASTRESGVPSRFSGSGS |
| | | GTDFTFTISSLQPEDIATYYCQNDYS |
| | | YPYTFGQGTKVEIK |
| SEQ ID NO: 231 | DNA VL | Gagatcgtcctgactcagtcacccgctaccct |
| | | gagcctgagccctggcgagcgggctacactgagctgta |
| | | aatctagtcagtcactgctggatagcggtaatcagaagaa |
| | | cttcctgacctggtatcagcagaagcccggtaaagcccct |
| | | aagctgctgatctactgggcctctactagagaatcaggcg |
| | | tgccctctaggtttagcggtagcggtagtggcaccgactt |
| | | caccttcactatctctagcctgcagcccgaggatatcgcta |
| | | cctactactgtcagaacgactatagctacccctacaccttc |
| | | ggtcaaggcactaaggtcgagattaag |
| SEQ ID NO: 190 | LC | EIVLTQSPATLSLSPGERATLS |
| | | CKSSQSLLDSGNQKNFLTWYQQKPG |
| | | KAPKLLIYWASTRESGVPSRFSGSGS |
| | | GTDFTFTISSLQPEDIATYYCQNDYS |
| | | YPYTFGQGTKVEIKRTVAAPSVFIFPP |
| | | SDEQLKSGTASVVCLLNNFYPREAK |
| | | VQWKVDNALQSGNSQESVTEQDSK |
| | | DSTYSLSSTLTLSKADYEKHKVYAC |
| | | EVTHQGLSSPVTKSFNRGEC |
| SEQ ID NO: 232 | DNA LC | Gagatcgtcctgactcagtcacccgctaccct |
| | | gagcctgagccctggcgagcgggctacactgagctgta |
| | | aatctagtcagtcactgctggatagcggtaatcagaagaa |
| | | cttcctgacctggtatcagcagaagcccggtaaagcccct |
| | | aagctgctgatctactgggcctctactagagaatcaggcg |
| | | tgccctctaggtttagcggtagcggtagtggcaccgactt |
| | | caccttcactatctctagcctgcagcccgaggatatcgcta |
| | | cctactactgtcagaacgactatagctacccctacaccttc |
| | | ggtcaaggcactaaggtcgagattaagcgtacggtggcc |
| | | gctcccagcgtgttcatcttcccccccagcgacgagcag |
| | | ctgaagagcggcaccgccagcgtggtgtgcctgctgaa |
| | | caacttctacccccgggaggccaaggtgcagtggaagg |
| | | tggacaacgccctgcagagcggcaacagccaggagag |
| | | cgtcaccgagcaggacagcaaggactccacctacagcc |
| | | tgagcagcaccctgaccctgagcaaggccgactacgag |
| | | aagcataaggtgtacgcctgcgaggtgacccaccaggg |
| | | cctgtccagccccgtgaccaagagcttcaacaggggcg |
| | | agtgc |
| BAP049-Clone-C HC | | |
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 | HCDR1 | GYTFTTY |
| (Chothia) | | |
| SEQ ID NO: 141 | HCDR2 | YPGTGG |
| (Chothia) | | |
| SEQ ID NO: 139 | HCD R3 | WTTGTGAY |
| (Chothia) | | |
| SEQ ID NO: 172 | VH | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSS |
| SEQ ID NO: 224 | DNA VH | GAAGTGCAGCTGGTGCAGTC |
| | | TGGCGCCGAAGTGAAGAAGCCTGG |
| | | CGAGTCCCTGCGGATCTCCTGCAA |
| | | GGGCTCTGGCTACACCTTCACCACC |
| | | TACTGGATGCACTGGGTGCGACAG |
| | | GCTACCGGCCAGGGCCTGGAATGG |
| | | ATGGGCAACATCTATCCTGGCACC |
| | | GGCGGCTCCAACTTCGACGAGAAG |
| | | TTCAAGAACAGAGTGACCATCACC |
| | | GCCGACAAGTCCACCTCCACCGCC |
| | | TACATGGAACTGTCCTCCCTGAGAT |
| | | CCGAGGACACCGCCGTGTACTACT |
| | | GCACCCGGTGGACAACCGGCACAG |
| | | GCGCTTATTGGGGCCAGGGCACCA |
| | | CAGTGACCGTGTCCTCT |
| SEQ ID NO: 225 | HC | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSSAS |
| | | TKGPSVFPLAPCSRSTSESTAALGCL |
| | | VKDYFPEPVTVSWNSGALTSGVHTF |
| | | PAVLQSSGLYSLSSVVTVPSSSLGTK |
| | | TYTCNVDHKPSNTKVDKRVESKYGP |
| | | PCPPCPAPEFLGGPSVFLFPPKPKDTL |
| | | MISRTPEVTCVVVDVSQEDPEVQFN |
| | | WYVDGVEVHNAKTKPREEQFNSTY |
| | | RVVSVLTVLHQDWLNGKEYKCKVS |
| | | NKGLPSSIEKTISKAKGQPREPQVYT |
| | | LPPSQEEMTKNQVSLTCLVKGFYPSD |
| | | IAVEWESNGQPENNYKTTPPVLDSD |
| | | GSFFLYSRLTVDKSRWQEGNVFSCS |
| | | VMHEALHNHYTQKSLSLSLG |
| SEQ ID NO: 226 | DNA HC | GAAGTGCAGCTGGTGCAGTC |
| | | TGGCGCCGAAGTGAAGAAGCCTGG |
| | | CGAGTCCCTGCGGATCTCCTGCAA |
| | | GGGCTCTGGCTACACCTTCACCACC |
| | | TACTGGATGCACTGGGTGCGACAG |
| | | GCTACCGGCCAGGGCCTGGAATGG |
| | | ATGGGCAACATCTATCCTGGCACC |
| | | GGCGGCTCCAACTTCGACGAGAAG |
| | | TTCAAGAACAGAGTGACCATCACC |
| | | GCCGACAAGTCCACCTCCACCGCC |
| | | TACATGGAACTGTCCTCCCTGAGAT |
| | | CCGAGGACACCGCCGTGTACTACT |
| | | GCACCCGGTGGACAACCGGCACAG |
| | | GCGCTTATTGGGGCCAGGGCACCA |
| | | CAGTGACCGTGTCCTCTGCTTCTAC |
| | | CAAGGGGCCCAGCGTGTTCCCCCT |
| | | GGCCCCCTGCTCCAGAAGCACCAG |
| | | CGAGAGCACAGCCGCCCTGGGCTG |
| | | CCTGGTGAAGGACTACTTCCCCGA |
| | | GCCCGTGACCGTGTCCTGGAACAG |
| | | CGGAGCCCTGACCAGCGGCGTGCA |
| | | CACCTTCCCCGCCGTGCTGCAGAGC |
| | | AGCGGCCTGTACAGCCTGAGCAGC |
| | | GTGGTGACCGTGCCCAGCAGCAGC |
| | | CTGGGCACCAAGACCTACACCTGT |
| | | AACGTGGACCACAAGCCCAGCAAC |
| | | ACCAAGGTGGACAAGAGGGTGGAG |
| | | AGCAAGTACGGCCCACCCTGCCCC |
| | | CCCTGCCCAGCCCCCGAGTTCCTGG |
| | | GCGGACCCAGCGTGTTCCTGTTCCC |
| | | CCCCAAGCCCAAGGACACCCTGAT |
| | | GATCAGCAGAACCCCCGAGGTGAC |
| | | CTGTGTGGTGGTGGACGTGTCCCA |
| | | GGAGGACCCCGAGGTCCAGTTCAA |
| | | CTGGTACGTGGACGGCGTGGAGGT |
| | | GCACAACGCCAAGACCAAGCCCAG |
| | | AGAGGAGCAGTTTAACAGCACCTA |
| | | CCGGGTGGTGTCCGTGCTGACCGT |
| | | GCTGCACCAGGACTGGCTGAACGG |
| | | CAAAGAGTACAAGTGTAAGGTCTC |
| | | CAACAAGGGCCTGCCAAGCAGCAT |
| | | CGAAAAGACCATCAGCAAGGCCAA |
| | | GGGCCAGCCTAGAGAGCCCCAGGT |
| | | CTACACCCTGCCACCCAGCCAAGA |
| | | GGAGATGACCAAGAACCAGGTGTC |
| | | CCTGACCTGTCTGGTGAAGGGCTTC |
| | | TACCCAAGCGACATCGCCGTGGAG |
| | | TGGGAGAGCAACGGCCAGCCCGAG |
| | | AACAACTACAAGACCACCCCCCCA |
| | | GTGCTGGACAGCGACGGCAGCTTC |
| | | TTCCTGTACAGCAGGCTGACCGTG |
| | | GACAAGTCCAGATGGCAGGAGGGC |
| | | AACGTCTTTAGCTGCTCCGTGATGC |
| | | ACGAGGCCCTGCACAACCACTACA |
| | | CCCAGAAGAGCCTGAGCCTGTCCC |
| | | TGGGC |
| BAP049-Clone-C LC | | |
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 | LCDR1 | SQSLLDSGNQKNF |
| (Chothia) | | |
| SEQ ID NO: 150 | LCDR2 | WAS |
| (Chothia) | | |
| SEQ ID NO: 167 | LCDR3 | DYSYPY |
| (Chothia) | | |
| SEQ ID NO: 200 | VL | EIVLTQSPDFQSVTPKEKVTIT |
| | | CKSSQSLLDSGNQKNFLTWYQQKPG |
| | | QAPRLLIYWASTRESGVPSRFSGSGS |
| | | GTDFTFTISSLEAEDAATYYCQNDYS |
| | | YPYTFGQGTKVEIK |
| SEQ ID NO: 233 | DNA VL | GAGATCGTGCTGACCCAGTC |
| | | CCCCGACTTCCAGTCCGTGACCCCC |
| | | AAAGAAAAAGTGACCATCACATGC |
| | | AAGTCCTCCCAGTCCCTGCTGGACT |
| | | CCGGCAACCAGAAGAACTTCCTGA |
| | | CCTGGTATCAGCAGAAGCCCGGCC |
| | | AGGCCCCCAGACTGCTGATCTACT |
| | | GGGCCTCCACCCGGGAATCTGGCG |
| | | TGCCCTCTAGATTCTCCGGCTCCGG |
| | | CTCTGGCACCGACTTTACCTTCACC |
| | | ATCTCCAGCCTGGAAGCCGAGGAC |
| | | GCCGCCACCTACTACTGCCAGAAC |
| | | GACTACTCCTACCCCTACACCTTCG |
| | | GCCAGGGCACCAAGGTGGAAATCA |
| | | AG |
| SEQ ID NO: 202 | LC | EIVLTQSPDFQSVTPKEKVTIT |
| | | CKSSQSLLDSGNQKNFLTWYQQKPG |
| | | QAPRLLIYWASTRESGVPSRFSGSGS |
| | | GTDFTFTISSLEAEDAATYYCQNDYS |
| | | YPYTFGQGTKVEIKRTVAAPSVFIFPP |
| | | SDEQLKSGTASVVCLLNNFYPREAK |
| | | VQWKVDNALQSGNSQESVTEQDSK |
| | | DSTYSLSSTLTLSKADYEKHKVYAC |
| | | EVTHQGLSSPVTKSFNRGEC |
| SEQ ID NO: 234 | DNA LC | GAGATCGTGCTGACCCAGTC |
| | | CCCCGACTTCCAGTCCGTGACCCCC |
| | | AAAGAAAAAGTGACCATCACATGC |
| | | AAGTCCTCCCAGTCCCTGCTGGACT |
| | | CCGGCAACCAGAAGAACTTCCTGA |
| | | CCTGGTATCAGCAGAAGCCCGGCC |
| | | AGGCCCCCAGACTGCTGATCTACT |
| | | GGGCCTCCACCCGGGAATCTGGCG |
| | | TGCCCTCTAGATTCTCCGGCTCCGG |
| | | CTCTGGCACCGACTTTACCTTCACC |
| | | ATCTCCAGCCTGGAAGCCGAGGAC |
| | | GCCGCCACCTACTACTGCCAGAAC |
| | | GACTACTCCTACCCCTACACCTTCG |
| | | GCCAGGGCACCAAGGTGGAAATCA |
| | | AGCGTACGGTGGCCGCTCCCAGCG |
| | | TGTTCATCTTCCCCCCAAGCGACGA |
| | | GCAGCTGAAGAGCGGCACCGCCAG |
| | | CGTGGTGTGTCTGCTGAACAACTTC |
| | | TACCCCAGGGAGGCCAAGGTGCAG |
| | | TGGAAGGTGGACAACGCCCTGCAG |
| | | AGCGGCAACAGCCAGGAGAGCGTC |
| | | ACCGAGCAGGACAGCAAGGACTCC |
| | | ACCTACAGCCTGAGCAGCACCCTG |
| | | ACCCTGAGCAAGGCCGACTACGAG |
| | | AAGCACAAGGTGTACGCCTGTGAG |
| | | GTGACCCACCAGGGCCTGTCCAGC |
| | | CCCGTGACCAAGAGCTTCAACAGG |
| | | GGCGAGTGC |
| BAP049-Clone-D HC | | |
| SEQ ID NO: 137 (Kabat) | HCDR1 | TYWMH |
| SEQ ID NO: 138 (Kabta) | HCDR2 | NIYPGTGGSNFDEKFKN |
| SEQ ID NO: 139 (Kabat) | HCDR3 | WITGTGAY |
| SEQ ID NO: 140 | HCDR1 | GYTFTTY |
| (Chothia) | | |
| SEQ ID NO: 141 | HCDR2 | YPGTGG |
| (Chothia) | | |
| SEQ ID NO: 139 | HCDR3 | WTTGTGAY |
| (Chothia) | | |
| SEQ ID NO: 184 | VH | EVQLVQSGAEVKKPGESLRIS |
| | | CKGSGYTFTTYWMHWIRQSPSRGLE |
| | | WLGNIYPGTGGSNFDEKFKNRFTISR |
| | | DNSKNTLYLQMNSLRAEDTAVYYC |
| | | TRWTTGTGAYWGQGTTVTVSS |
| SEQ ID NO: 235 | DNA VH | GAAGTGCAGCTGGTGCAGTC |
| | | TGGCGCCGAAGTGAAGAAGCCTGG |
| | | CGAGTCCCTGCGGATCTCCTGCAA |
| | | GGGCTCTGGCTACACCTTCACCACC |
| | | TACTGGATGCACTGGATCCGGCAG |
| | | TCCCCCTCTAGGGGCCTGGAATGG |
| | | CTGGGCAACATCTACCCTGGCACC |
| | | GGCGGCTCCAACTTCGACGAGAAG |
| | | TTCAAGAACAGGTTCACCATCTCCC |
| | | GGGACAACTCCAAGAACACCCTGT |
| | | ACCTGCAGATGAACTCCCTGCGGG |
| | | CCGAGGACACCGCCGTGTACTACT |
| | | GTACCAGATGGACCACCGGAACCG |
| | | GCGCCTATTGGGGCCAGGGCACAA |
| | | CAGTGACCGTGTCCTCC |
| SEQ ID NO: 236 | HC | EVQLVQSGAEVKKPGESLRIS |
| | | CKGSGYTFTTYWMHWIRQSPSRGLE |
| | | WLGNIYPGTGGSNFDEKFKNRFTISR |
| | | DNSKNTLYLQMNSLRAEDTAVYYC |
| | | TRWTTGTGAYWGQGTTVTVSSAST |
| | | KGPSVFPLAPCSRSTSESTAALGCLV |
| | | KDYFPEPVTVSWNSGALTSGVHTFP |
| | | AVLQSSGLYSLSSVVTVPSSSLGTKT |
| | | YTCNVDHKPSNTKVDKRVESKYGPP |
| | | CPPCPAPEFLGGPSVFLFPPKPKDTL |
| | | MISRTPEVTCVVVDVSQEDPEVQFN |
| | | WYVDGVEVHNAKTKPREEQFNSTY |
| | | RVVSVLTVLHQDWLNGKEYKCKVS |
| | | NKGLPSSIEKTISKAKGQPREPQVYT |
| | | LPPSQEEMTKNQVSLTCLVKGFYPSD |
| | | IAVEWESNGQPENNYKTTPPVLDSD |
| | | GSFFLYSRLTVDKSRWQEGNVFSCS |
| | | VMHEALHNHYTQKSLSLSLG |
| SEQ ID NO: 237 | DNA HC | GAAGTGCAGCTGGTGCAGTC |
| | | TGGCGCCGAAGTGAAGAAGCCTGG |
| | | CGAGTCCCTGCGGATCTCCTGCAA |
| | | GGGCTCTGGCTACACCTTCACCACC |
| | | TACTGGATGCACTGGATCCGGCAG |
| | | TCCCCCTCTAGGGGCCTGGAATGG |
| | | CTGGGCAACATCTACCCTGGCACC |
| | | GGCGGCTCCAACTTCGACGAGAAG |
| | | TTCAAGAACAGGTTCACCATCTCCC |
| | | GGGACAACTCCAAGAACACCCTGT |
| | | ACCTGCAGATGAACTCCCTGCGGG |
| | | CCGAGGACACCGCCGTGTACTACT |
| | | GTACCAGATGGACCACCGGAACCG |
| | | GCGCCTATTGGGGCCAGGGCACAA |
| | | CAGTGACCGTGTCCTCCGCTTCTAC |
| | | CAAGGGGCCCAGCGTGTTCCCCCT |
| | | GGCCCCCTGCTCCAGAAGCACCAG |
| | | CGAGAGCACAGCCGCCCTGGGCTG |
| | | CCTGGTGAAGGACTACTTCCCCGA |
| | | GCCCGTGACCGTGTCCTGGAACAG |
| | | CGGAGCCCTGACCAGCGGCGTGCA |
| | | CACCTTCCCCGCCGTGCTGCAGAGC |
| | | AGCGGCCTGTACAGCCTGAGCAGC |
| | | GTGGTGACCGTGCCCAGCAGCAGC |
| | | CTGGGCACCAAGACCTACACCTGT |
| | | AACGTGGACCACAAGCCCAGCAAC |
| | | ACCAAGGTGGACAAGAGGGTGGAG |
| | | AGCAAGTACGGCCCACCCTGCCCC |
| | | CCCTGCCCAGCCCCCGAGTTCCTGG |
| | | GCGGACCCAGCGTGTTCCTGTTCCC |
| | | CCCCAAGCCCAAGGACACCCTGAT |
| | | GATCAGCAGAACCCCCGAGGTGAC |
| | | CTGTGTGGTGGTGGACGTGTCCCA |
| | | GGAGGACCCCGAGGTCCAGTTCAA |
| | | CTGGTACGTGGACGGCGTGGAGGT |
| | | GCACAACGCCAAGACCAAGCCCAG |
| | | AGAGGAGCAGTTTAACAGCACCTA |
| | | CCGGGTGGTGTCCGTGCTGACCGT |
| | | GCTGCACCAGGACTGGCTGAACGG |
| | | CAAAGAGTACAAGTGTAAGGTCTC |
| | | CAACAAGGGCCTGCCAAGCAGCAT |
| | | CGAAAAGACCATCAGCAAGGCCAA |
| | | GGGCCAGCCTAGAGAGCCCCAGGT |
| | | CTACACCCTGCCACCCAGCCAAGA |
| | | GGAGATGACCAAGAACCAGGTGTC |
| | | CCTGACCTGTCTGGTGAAGGGCTTC |
| | | TACCCAAGCGACATCGCCGTGGAG |
| | | TGGGAGAGCAACGGCCAGCCCGAG |
| | | AACAACTACAAGACCACCCCCCCA |
| | | GTGCTGGACAGCGACGGCAGCTTC |
| | | TTCCTGTACAGCAGGCTGACCGTG |
| | | GACAAGTCCAGATGGCAGGAGGGC |
| | | AACGTCTTTAGCTGCTCCGTGATGC |
| | | ACGAGGCCCTGCACAACCACTACA |
| | | CCCAGAAGAGCCTGAGCCTGTCCC |
| | | TGGGC |
| BAP049-Clone-D LC | | |
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 | LCDR1 | SQSLLDSGNQKNF |
| (Chothia) | | |
| SEQ ID NO: 150 | LCDR2 | WAS |
| (Chothia) | | |
| SEQ ID NO: 167 | LCDR3 | DYSYPY |
| (Chothia) | | |
| SEQ ID NO: 204 | VL | EIVLTQSPATLSLSPGERATLS |
| | | CKSSQSLLDSGNQKNFLTWYQQKPG |
| | | QAPRLLIYWASTRESGVPSRFSGSGS |
| | | GTDFTFTISSLEAEDAATYYCQNDYS |
| | | YPYTFGQGTKVEIK |
| SEQ ID NO: 238 | DNA VL | GAGATCGTGCTGACCCAGTC |
| | | CCCTGCCACCCTGTCACTGTCTCCA |
| | | GGCGAGAGAGCTACCCTGTCCTGC |
| | | AAGTCCTCCCAGTCCCTGCTGGACT |
| | | CCGGCAACCAGAAGAACTTCCTGA |
| | | CCTGGTATCAGCAGAAGCCCGGCC |
| | | AGGCCCCCAGACTGCTGATCTACT |
| | | GGGCCTCCACCCGGGAATCTGGCG |
| | | TGCCCTCTAGATTCTCCGGCTCCGG |
| | | CTCTGGCACCGACTTTACCTTCACC |
| | | ATCTCCAGCCTGGAAGCCGAGGAC |
| | | GCCGCCACCTACTACTGCCAGAAC |
| | | GACTACTCCTACCCCTACACCTTCG |
| | | GCCAGGGCACCAAGGTGGAAATCA |
| | | AG |
| SEQ ID NO: 206 | LC | EIVLTQSPATLSLSPGERATLS |
| | | CKSSQSLLDSGNQKNFLTWYQQKPG |
| | | QAPRLLIYWASTRESGVPSRFSGSGS |
| | | GTDFTFTISSLEAEDAATYYCQNDYS |
| | | YPYTFGQGTKVEIKRTVAAPSVFIFPP |
| | | SDEQLKSGTASVVCLLNNFYPREAK |
| | | VQWKVDNALQSGNSQESVTEQDSK |
| | | DSTYSLSSTLTLSKADYEKHKVYAC |
| | | EVTHQGLSSPVTKSFNRGEC |
| SEQ ID NO: 239 | DNA LC | GAGATCGTGCTGACCCAGTC |
| | | CCCTGCCACCCTGTCACTGTCTCCA |
| | | GGCGAGAGAGCTACCCTGTCCTGC |
| | | AAGTCCTCCCAGTCCCTGCTGGACT |
| | | CCGGCAACCAGAAGAACTTCCTGA |
| | | CCTGGTATCAGCAGAAGCCCGGCC |
| | | AGGCCCCCAGACTGCTGATCTACT |
| | | GGGCCTCCACCCGGGAATCTGGCG |
| | | TGCCCTCTAGATTCTCCGGCTCCGG |
| | | CTCTGGCACCGACTTTACCTTCACC |
| | | ATCTCCAGCCTGGAAGCCGAGGAC |
| | | GCCGCCACCTACTACTGCCAGAAC |
| | | GACTACTCCTACCCCTACACCTTCG |
| | | GCCAGGGCACCAAGGTGGAAATCA |
| | | AGCGTACGGTGGCCGCTCCCAGCG |
| | | TGTTCATCTTCCCCCCAAGCGACGA |
| | | GCAGCTGAAGAGCGGCACCGCCAG |
| | | CGTGGTGTGTCTGCTGAACAACTTC |
| | | TACCCCAGGGAGGCCAAGGTGCAG |
| | | TGGAAGGTGGACAACGCCCTGCAG |
| | | AGCGGCAACAGCCAGGAGAGCGTC |
| | | ACCGAGCAGGACAGCAAGGACTCC |
| | | ACCTACAGCCTGAGCAGCACCCTG |
| | | ACCCTGAGCAAGGCCGACTACGAG |
| | | AAGCACAAGGTGTACGCCTGTGAG |
| | | GTGACCCACCAGGGCCTGTCCAGC |
| | | CCCGTGACCAAGAGCTTCAACAGG |
| | | GGCGAGTGC |
| BAP049-Clone-E HC | | |
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 | HCDR1 | GYTFTTY |
| (Chothia) | | |
| SEQ ID NO: 141 | HCDR2 | YPGTGG |
| (Chothia) | | |
| SEQ ID NO: 139 | HCDR3 | WTTGTGAY |
| (Chothia) | | |
| SEQ ID NO: 172 | VH | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSS |
| SEQ ID NO: 229 | DNA VH | Gaggtgcagctggtgcagtcaggcgccgaa |
| | | gtgaagaagcccggcgagtcactgagaattagctgtaaa |
| | | ggttcaggctacaccttcactacctactggatgcactgggt |
| | | ccgccaggctaccggtcaaggcctcgagtggatgggta |
| | | atatctaccccggcaccggcggctctaacttcgacgaga |
| | | agtttaagaatagagtgactatcaccgccgataagtctact |
| | | agcaccgcctatatggaactgtctagcctgagatcagag |
| | | gacaccgccgtctactactgcactaggtggactaccggc |
| | | acaggcgcctactggggtcaaggcactaccgtgaccgt |
| | | gtctagc |
| SEQ ID NO: 225 | HC | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSSAS |
| | | TKGPSVFPLAPCSRSTSESTAALGCL |
| | | VKDYFPEPVTVSWNSGALTSGVHTF |
| | | PAVLQSSGLYSLSSVVTVPSSSLGTK |
| | | TYTCNVDHKPSNTKVDKRVESKYGP |
| | | PCPPCPAPEFLGGPSVFLFPPKPKDTL |
| | | MISRTPEVTCVVVDVSQEDPEVQFN |
| | | WYVDGVEVHNAKTKPREEQFNSTY |
| | | RVVSVLTVLHQDWLNGKEYKCKVS |
| | | NKGLPSSIEKTISKAKGQPREPQVYT |
| | | LPPSQEEMTKNQVSLTCLVKGFYPSD |
| | | IAVEWESNGQPENNYKTTPPVLDSD |
| | | GSFFLYSRLTVDKSRWQEGNVFSCS |
| | | VMHEALHNHYTQKSLSLSLG |
| SEQ ID NO: 230 | DNA HC | gaggtgcagctggtgcagtcaggcgccgaag |
| | | tgaagaagcccggcgagtcactgagaattagctgtaaag |
| | | gttcaggctacaccttcactacctactggatgcactgggtc |
| | | cgccaggctaccggtcaaggcctcgagtggatgggtaat |
| | | atctaccccggcaccggcggctctaacttcgacgagaag |
| | | tttaagaatagagtgactatcaccgccgataagtctactag |
| | | caccgcctatatggaactgtctagcctgagatcagagga |
| | | caccgccgtctactactgcactaggtggactaccggcac |
| | | aggcgcctactggggtcaaggcactaccgtgaccgtgtc |
| | | tagcgctagcactaagggcccgtccgtgttccccctggc |
| | | accttgtagccggagcactagcgaatccaccgctgccct |
| | | cggctgcctggtcaaggattacttcccggagcccgtgac |
| | | cgtgtcctggaacagcggagccctgacctccggagtgc |
| | | acaccttccccgctgtgctgcagagctccgggctgtactc |
| | | gctgtcgtcggtggtcacggtgccttcatctagcctgggt |
| | | accaagacctacacttgcaacgtggaccacaagccttcc |
| | | aacactaaggtggacaagcgcgtcgaatcgaagtacgg |
| | | cccaccgtgcccgccttgtcccgcgccggagttcctcgg |
| | | cggtccctcggtctttctgttcccaccgaagcccaaggac |
| | | actttgatgatttcccgcacccctgaagtgacatgcgtggt |
| | | cgtggacgtgtcacaggaagatccggaggtgcagttcaa |
| | | ttggtacgtggatggcgtcgaggtgcacaacgccaaaac |
| | | caagccgagggaggagcagttcaactccacttaccgcgt |
| | | cgtgtccgtgctgacggtgctgcatcaggactggctgaa |
| | | cgggaaggagtacaagtgcaaagtgtccaacaagggac |
| | | ttcctagctcaatcgaaaagaccatctcgaaagccaagg |
| | | gacagccccgggaaccccaagtgtataccctgccaccg |
| | | agccaggaagaaatgactaagaaccaagtctcattgactt |
| | | gccttgtgaagggcttctacccatcggatatcgccgtgga |
| | | atgggagtccaacggccagccggaaaacaactacaaga |
| | | ccacccctccggtgctggactcagacggatccttcttcct |
| | | ctactcgcggctgaccgtggataagagcagatggcagg |
| | | agggaaatgtgttcagctgttctgtgatgcatgaagccctg |
| | | cacaaccactacactcagaagtccctgtccctctccctgg |
| | | ga |
| BAP049-Clone-E LC | | |
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 | LCDR1 | SQSLLDSGNQKNF |
| (Chothia) | | |
| SEQ ID NO: 150 | LCDR2 | WAS |
| (Chothia) | | |
| SEQ ID NO: 167 | LCDR3 | DYSYPY |
| (Chothia) | | |
| SEQ ID NO: 204 | VL | EIVLTQSPATLSLSPGERATLS |
| | | CKSSQSLLDSGNQKNFLTWYQQKPG |
| | | QAPRLLIYWASTRESGVPSRFSGSGS |
| | | GTDFTFTISSLEAEDAATYYCQNDYS |
| | | YPYTFGQGTKVEIK |
| SEQ ID NO: 240 | DNA VL | Gagatcgtcctgactcagtcacccgctaccct |
| | | gagcctgagccctggcgagcgggctacactgagctgta |
| | | aatctagtcagtcactgctggatagcggtaatcagaagaa |
| | | cttcctgacctggtatcagcagaagcccggtcaagcccct |
| | | agactgctgatctactgggcctctactagagaatcaggcg |
| | | tgccctctaggtttagcggtagcggtagtggcaccgactt |
| | | caccttcactatctctagcctggaagccgaggacgccgct |
| | | acctactactgtcagaacgactatagctacccctacacctt |
| | | cggtcaaggcactaaggtcgagattaag |
| SEQ ID NO: 206 | LC | EIVLTQSPATLSLSPGERATLS |
| | | CKSSQSLLDSGNQKNFLTWYQQKPG |
| | | QAPRLLIYWASTRESGVPSRFSGSGS |
| | | GTDFTFTISSLEAEDAATYYCQNDYS |
| | | YPYTFGQGTKVEIKRTVAAPSVFIFPP |
| | | SDEQLKSGTASVVCLLNNFYPREAK |
| | | VQWKVDNALQSGNSQESVTEQDSK |
| | | DSTYSLSSTLTLSKADYEKHKVYAC |
| | | EVTHQGLSSPVTKSFNRGEC |
| SEQ ID NO: 241 | DNA LC | Gagatcgtcctgactcagtcacccgctaccct |
| | | gagcctgagccctggcgagcgggctacactgagctgta |
| | | aatctagtcagtcactgctggatagcggtaatcagaagaa |
| | | cttcctgacctggtatcagcagaagcccggtcaagcccct |
| | | agactgctgatctactgggcctctactagagaatcaggcg |
| | | tgccctctaggtttagcggtagcggtagtggcaccgactt |
| | | caccttcactatctctagcctggaagccgaggacgccgct |
| | | acctactactgtcagaacgactatagctacccctacacctt |
| | | cggtcaaggcactaaggtcgagattaagcgtacggtggc |
| | | cgctcccagcgtgttcatcttcccccccagcgacgagca |
| | | gctgaagagcggcaccgccagcgtggtgtgcctgctga |
| | | acaacttctacccccgggaggccaaggtgcagtggaag |
| | | gtggacaacgccctgcagagcggcaacagccaggaga |
| | | gcgtcaccgagcaggacagcaaggactccacctacagc |
| | | ctgagcagcaccctgaccctgagcaaggccgactacga |
| | | gaagcataaggtgtacgcctgcgaggtgacccaccagg |
| | | gcctgtccagccccgtgaccaagagcttcaacaggggc |
| | | gagtgc |
| BAP049 HC | | |
| SEQ ID NO: 242 (Kabat) | HCDR1 | ACTTACTGGATGCAC |
| SEQ ID NO: 243 (Kabat) | HCDR2 | AATATTTATCCTGGTACTGG |
| | | TGGTTCTAACTTCGATGAGAAGTTC |
| | | AAGAAC |
| SEQ ID NO: 244 (Kabat) | HCDR3 | TGGACTACTGGGACGGGAGC |
| | | TTAT |
| SEQ ID NO: 245 | HCDR1 | GGCTACACATTCACCACTTA |
| (Chothia) | | C |
| SEQ ID NO: 246 | HCDR2 | TATCCTGGTACTGGTGGT |
| (Chothia) | | |
| SEQ ID NO: 244 | HCDR3 | TGGACTACTGGGACGGGAGC |
| (Chothia) | | TTAT |
| BAP049 LC | | |
| SEQ ID NO: 247 (Kabat) | LCDR1 | AAGTCCAGTCAGAGTCTGTT |
| | | AGACAGTGGAAATCAAAAGAACTT |
| | | CTTGACC |
| SEQ ID NO: 248 (Kabat) | LCDR2 | TGGGCATCCACTAGGGAATC |
| | | T |
| SEQ ID NO: 249 (Kabat) | LCDR3 | CAGAATGATTATAGTTATCC |
| | | GTGCACG |
| SEQ ID NO: 250 | LCDR1 | AGTCAGAGTCTGTTAGACAG |
| (Chothia) | | TGGAAATCAAAAGAACTTC |
| SEQ ID NO: 251 | LCDR2 | TGGGCATCC |
| (Chothia) | | |
| SEQ ID NO: 252 | LCDR3 | GATTATAGTTATCCGTGC |
| (Chothia) | | |
| BAP049-chi HC | | |
| SEQ ID NO: 242 (Kabat) | HCDR1 | ACTTACTGGATGCAC |
| SEQ ID NO: 243 (Kabat) | HCDR2 | AATATTTATCCTGGTACTGG |
| | | TGGTTCTAACTTCGATGAGAAGTTC |
| | | AAGAAC |
| SEQ ID NO: 244 (Kabat) | HCDR3 | TGGACTACTGGGACGGGAGC |
| | | TTAT |
| SEQ ID NO: 245 | HCDR1 | GGCTACACATTCACCACTTA |
| (Chothia) | | C |
| SEQ ID NO: 246 | HCDR2 | TATCCTGGTACTGGTGGT |
| (Chothia) | | |
| SEQ ID NO: 244 | HCDR3 | TGGACTACTGGGACGGGAGC |
| (Chothia) | | TTAT |
| BAP049-chi LC | | |
| SEQ ID NO: 247 (Kabat) | LCDR1 | AAGTCCAGTCAGAGTCTGTT |
| | | AGACAGTGGAAATCAAAAGAACTT |
| | | CTTGACC |
| SEQ ID NO: 248 (Kabat) | LCDR2 | TGGGCATCCACTAGGGAATC |
| | | T |
| SEQ ID NO: 249 (Kabat) | LCDR3 | CAGAATGATTATAGTTATCC |
| | | GTGCACG |
| SEQ ID NO: 250 | LCDR1 | AGTCAGAGTCTGTTAGACAG |
| (Chothia) | | TGGAAATCAAAAGAACTTC |
| SEQ ID NO: 251 | LCDR2 | TGGGCATCC |
| (Chothia) | | |
| SEQ ID NO: 252 | LCDR3 | GATTATAGTTATCCGTGC |
| (Chothia) | | |
| BAP049-chi Y HC | | |
| SEQ ID NO: 242 (Kabat) | HCDR1 | ACTTACTGGATGCAC |
| SEQ ID NO: 243 (Kabat) | HCDR2 | AATATTTATCCTGGTACTGG |
| | | TGGTTCTAACTTCGATGAGAAGTTC |
| | | AAGAAC |
| SEQ ID NO: 244 (Kabat) | HCDR3 | TGGACTACTGGGACGGAGC |
| | | TTAT |
| SEQ ID NO: 245 | HCDR1 | GGCTACACATTCACCACTTA |
| (Chothia) | | C |
| SEQ ID NO: 246 | HCDR2 | TATCCTGGTACTGGTGGT |
| (Chothia) | | |
| SEQ ID NO: 244 | HCDR3 | TGGACTACTGGGACGGGAGC |
| (Chothia) | | TTAT |
| BAP049-chi Y LC | | |
| SEQ ID NO: 247 (Kabat) | LCDR1 | AAGTCCAGTCAGAGTCTGTT |
| | | AGACAGTGGAAATCAAAAGAACTT |
| | | CTTGACC |
| SEQ ID NO: 248 (Kabat) | LCDR2 | TGGGCATCCACTAGGGAATC |
| | | T |
| SEQ ID NO: 253 (Kabat) | LCDR3 | CAGAATGATTATAGTTATCC |
| | | GTACACG |
| SEQ ID NO: 250 | LCDR1 | AGTCAGAGTCTGTTAGACAG |
| (Chothia) | | TGGAAATCAAAAGAACTTC |
| SEQ ID NO: 251 | LCDR2 | TGGGCATCC |
| (Chothia) | | |
| SEQ ID NO: 254 | LCDR3 | GATTATAGTTATCCGTAC |
| (Chothia) | | |
| BAP049-hum01 HC | | |
| SEQ ID NO: 242 (Kabat) | HCDR1 | ACTTACTGGATGCAC |
| SEQ ID NO: 243 (Kabat) | HCDR2 | AATATTTATCCTGGTACTGG |
| | | TGGTTCTAACTTCGATGAGAAGTTC |
| | | AAGAAC |
| SEQ ID NO: 244 (Kabat) | HCDR3 | TGGACTACTGGGACGGGAGC |
| | | TTAT |
| SEQ ID NO: 245 | HCDR1 | GGCTACACATTCACCACTTA |
| (Chothia) | | C |
| SEQ ID NO: 246 | HCDR2 | TATCCTGGTACTGGTGGT |
| (Chothia) | | |
| SEQ ID NO: 244 | HCDR3 | TGGACTACTGGGACGGGAGC |
| (Chothia) | | TTAT |
| BAP049-hum01 LC | | |
| SEQ ID NO: 247 (Kabat) | LCDR1 | AAGTCCAGTCAGAGTCTGTT |
| | | AGACAGTGGAAATCAAAAGAACTT |
| | | CTTGACC |
| SEQ ID NO: 248 (Kabat) | LCDR2 | TGGGCATCCACTAGGGAATC |
| | | T |
| SEQ ID NO: 253 (Kabat) | LCDR3 | CAGAATGATTATAGTTATCC |
| | | GTACACG |
| SEQ ID NO: 250 | LCDR1 | AGTCAGAGTCTGTTAGACAG |
| (Chothia) | | TGGAAATCAAAAGAACTTC |
| SEQ ID NO: 251 | LCDR2 | TGGGCATCC |
| (Chothia) | | |
| SEQ ID NO: 254 | LCDR3 | GATTATAGTTATCCGTAC |
| (Chothia) | | |
| BAP049-hum02 HC | | |
| SEQ ID NO: 242 (Kabat) | HCDR1 | ACTTACTGGATGCAC |
| SEQ ID NO: 243 (Kabat) | HCDR2 | AATATTTATCCTGGTACTGG |
| | | TGGTTCTAACTTCGATGAGAAGTTC |
| | | AAGAAC |
| SEQ ID NO: 244 (Kabat) | HCDR3 | TGGACTACTGGGACGGGAGC |
| | | TTAT |
| SEQ ID NO: 245 | HCDR1 | GGCTACACATTCACCACTTA |
| (Chothia) | | C |
| SEQ ID NO: 246 | HCDR2 | TATCCTGGTACTGGTGGT |
| (Chothia) | | |
| SEQ ID NO: 244 | HCDR3 | TGGACTACTGGGACGGGAGC |
| (Chothia) | | TTAT |
| BAP049-hum02 LC | | |
| SEQ ID NO: 247 (Kabat) | LCDR1 | AAGTCCAGTCAGAGTCTGTT |
| | | AGACAGTGGAAATCAAAAGAACTT |
| | | CTTGACC |
| SEQ ID NO: 248 (Kabat) | LCDR2 | TGGGCATCCACTAGGGAATC |
| | | T |
| SEQ ID NO: 253 (Kabat) | LCDR3 | CAGAATGATTATAGTTATCC |
| | | GTACACG |
| SEQ ID NO: 250 | LCDR1 | AGTCAGAGTCTGTTAGACAG |
| (Chothia) | | TGGAAATCAAAAGAACTTC |
| SEQ ID NO: 251 | LCDR2 | TGGGCATCC |
| (Chothia) | | |
| SEQ ID NO: 254 | LCDR3 | GATTATAGTTATCCGTAC |
| (Chothia) | | |
| BAP049-hum03 HC | | |
| SEQ ID NO: 242 (Kabat) | HCDR1 | ACTTACTGGATGCAC |
| SEQ ID NO: 243 (Kabat) | HCDR2 | AATATTTATCCTGGTACTGG |
| | | TGGTTCTAACTTCGATGAGAAGTTC |
| | | AAGAAC |
| SEQ ID NO: 244 (Kabat) | HCDR3 | TGGACTACTGGGACGGGAGC |
| | | TTAT |
| SEQ ID NO: 245 | HCDR1 | GGCTACACATTCACCACTTA |
| (Chothia) | | C |
| SEQ ID NO: 246 | HCDR2 | TATCCTGGTACTGGTGGT |
| (Chothia) | | |
| SEQ ID NO: 244 | HCDR3 | TGGACTACTGGGACGGGAGC |
| (Chothia) | | TTAT |
| BAP049-hum03 LC | | |
| SEQ ID NO: 247 (Kabat) | LCDR1 | AAGTCCAGTCAGAGTCTGTT |
| | | AGACAGTGGAAATCAAAAGAACTT |
| | | CTTGACC |
| SEQ ID NO: 248 (Kabat) | LCDR2 | TGGGCATCCACTAGGGAATC |
| | | T |
| SEQ ID NO: 253 (Kabat) | LCDR3 | CAGAATGATTATAGTTATCC |
| | | GTACACG |
| SEQ ID NO: 250 | LCDR1 | AGTCAGAGTCTGTTAGACAG |
| (Chothia) | | TGGAAATCAAAAGAACTTC |
| SEQ ID NO: 251 | LCDR2 | TGGGCATCC |
| (Chothia) | | |
| SEQ ID NO: 254 | LCDR3 | GATTATAGTTATCCGTAC |
| (Chothia) | | |
| BAP049-hum04 HC | | |
| SEQ ID NO: 242 (Kabat) | HCDR1 | ACTTACTGGATGCAC |
| SEQ ID NO: 243 (Kabat) | HCDR2 | AATATTTATCCTGGTACTGG |
| | | TGGTTCTAACTTCGATGAGAAGTTC |
| | | AAGAAC |
| SEQ ID NO: 244 (Kabat) | HCDR3 | TGGACTACTGGGACGGGAGC |
| | | TTAT |
| SEQ ID NO: 245 | HCDR1 | GGCTACACATTCACCACTTA |
| (Chothia) | | C |
| SEQ ID NO: 246 | HCDR2 | TATCCTGGTACTGGTGGT |
| (Chothia) | | |
| SEQ ID NO: 244 | HCDR3 | TGGACTACTGGGACGGGAGC |
| (Chothia) | | TTAT |
| BAP049-hum04 LC | | |
| SEQ ID NO: 247 (Kabat) | LCDR1 | AAGTCCAGTCAGAGTCTGTT |
| | | AGACAGTGGAAATCAAAAGAACTT |
| | | CTTGACC |
| SEQ ID NO: 248 (Kabat) | LCDR2 | TGGGCATCCACTAGGGAATC |
| | | T |
| SEQ ID NO: 253 (Kabat) | LCDR3 | CAGAATGATTATAGTTATCC |
| | | GTACACG |
| SEQ ID NO: 250 | LCDR1 | AGTCAGAGTCTGTTAGACAG |
| (Chothia) | | TGGAAATCAAAAGAACTTC |
| SEQ ID NO: 251 | LCDR2 | TGGGCATCC |
| (Chothia) | | |
| SEQ ID NO: 254 | LCDR3 | |
| (Chothia) | | GATTATAGTTATCCGTAC |
| BAP049-hum05 HC | | |
| SEQ ID NO: 242 (Kabat) | HCDR1 | ACTTACTGGATGCAC |
| SEQ ID NO: 243 (Kabat) | HCDR2 | AATATTTATCCTGGTACTGG |
| | | TGGTTCTAACTTCGATGAGAAGTTC |
| | | AAGAAC |
| SEQ ID NO: 244 (Kabat) | HCDR3 | TGGACTACTGGGACGGGAGC |
| | | TTAT |
| SEQ ID NO: 245 | HCDR1 | GGCTACACATTCACCACTTA |
| (Chothia) | | C |
| SEQ ID NO: 246 | HCDR2 | TATCCTGGTACTGGTGGT |
| (Chothia) | | |
| SEQ ID NO: 244 | HCDR3 | TGGACTACTGGGACGGGAGC |
| (Chothia) | | TTAT |
| BAP049-hum05 LC | | |
| SEQ ID NO: 247 (Kabat) | LCDR1 | AAGTCCAGTCAGAGTCTGTT |
| | | AGACAGTGGAAATCAAAAGAACTT |
| | | CTTGACC |
| SEQ ID NO: 248 (Kabat) | LCDR2 | TGGGCATCCACTAGGGAATC |
| | | T |
| SEQ ID NO: 253 (Kabat) | LCDR3 | CAGAATGATTATAGTTATCC |
| | | GTACACG |
| SEQ ID NO: 250 | LCDR1 | AGTCAGAGTCTGTTAGACAG |
| (Chothia) | | TGGAAATCAAAAGAACTTC |
| SEQ ID NO: 251 | LCDR2 | TGGGCATCC |
| (Chothia) | | |
| SEQ ID NO: 254 | LCDR3 | |
| (Chothia) | | GATTATAGTTATCCGTAC |
| BAP049-hum06 HC | | |
| SEQ ID NO: 242 (Kabat) | HCDR1 | ACTTACTGGATGCAC |
| SEQ ID NO: 243 (Kabat) | HCDR2 | AATATTTATCCTGGTACTGG |
| | | TGGTTCTAACTTCGATGAGAAGTTC |
| | | AAGAAC |
| SEQ ID NO: 244 (Kabat) | HCDR3 | TGGACTACTGGGACGGGAGC |
| | | TTAT |
| SEQ ID NO: 245 | HCDR1 | GGCTACACATTCACCACTTA |
| (Chothia) | | C |
| SEQ ID NO: 246 | HCDR2 | TATCCTGGTACTGGTGGT |
| (Chothia) | | |
| SEQ ID NO: 244 | HCDR3 | TGGACTACTGGGACGGGAGC |
| (Chothia) | | TTAT |
| BAP049-hum06 LC | | |
| SEQ ID NO: 247 (Kabat) | LCDR1 | AAGTCCAGTCAGAGTCTGTT |
| | | AGACAGTGGAAATCAAAAGAACTT |
| | | CTTGACC |
| SEQ ID NO: 248 (Kabat) | LCDR2 | TGGGCATCCACTAGGGAATC |
| | | T |
| SEQ ID NO: 253 (Kabat) | LCDR3 | CAGAATGATTATAGTTATCC |
| | | GTACACG |
| SEQ ID NO: 250 | LCDR1 | AGTCAGAGTCTGTTAGACAG |
| (Chothia) | | TGGAAATCAAAAGAACTTC |
| SEQ ID NO: 251 | LCDR2 | TGGGCATCC |
| (Chothia) | | |
| SEQ ID NO: 254 | LCDR3 | GATTATAGTTATCCGTAC |
| (Chothia) | | |
| BAP049-hum07 HC | | |
| SEQ ID NO: 242 (Kabat) | HCDR1 | ACTTACTGGATGCAC |
| SEQ ID NO: 243 (Kabat) | HCDR2 | AATATTTATCCTGGTACTGG |
| | | TGGTTCTAACTTCGATGAGAAGTTC |
| | | AAGAAC |
| SEQ ID NO: 244 (Kabat) | HCDR3 | TGGACTACTGGGACGGGAGC |
| | | TTAT |
| SEQ ID NO: 245 | HCDR1 | GGCTACACATTCACCACTTA |
| (Chothia) | | |
| SEQ ID NO: 246 | HCDR2 | TATCCTGGTACTGGTGGT |
| (Chothia) | | |
| SEQ ID NO: 244 | HCDR3 | TGGACTACTGGGACGGGAGC |
| (Chothia) | | TTAT |
| BAP049-hum07 LC | | |
| SEQ ID NO: 247 (Kabat) | LCDR1 | AAGTCCAGTCAGAGTCTGTT |
| | | AGACAGTGGAAATCAAAAGAACTT |
| | | CTTGACC |
| SEQ ID NO: 248 (Kabat) | LCDR2 | TGGGCATCCACTAGGGAATC |
| | | T |
| SEQ ID NO: 253 (Kabat) | LCDR3 | CAGAATGATTATAGTTATCC |
| | | GTACACG |
| SEQ ID NO: 250 | LCDR1 | AGTCAGAGTCTGTTAGACAG |
| (Chothia) | | TGGAAATCAAAAGAACTTC |
| SEQ ID NO: 251 | LCDR2 | TGGGCATCC |
| (Chothia) | | |
| SEQ ID NO: 254 | LCDR3 | |
| (Chothia) | | GATTATAGTTATCCGTAC |
| BAP049-hum08 HC | | |
| SEQ ID NO: 242 (Kabat) | HCDR1 | ACTTACTGGATGCAC |
| SEQ ID NO: 243 (Kabat) | HCDR2 | AATATTTATCCTGGTACTGG |
| | | TGGTTCTAACTTCGATGAGAAGTTC |
| | | AAGAAC |
| SEQ ID NO: 244 (Kabat) | HCDR3 | TGGACTACTGGGACGGGAGC |
| | | TTAT |
| SEQ ID NO: 245 | HCDR1 | GGCTACACATTCACCACTTA |
| (Chothia) | | C |
| SEQ ID NO: 246 | HCDR2 | TATCCTGGTACTGGTGGT |
| (Chothia) | | |
| SEQ ID NO: 244 | HCDR3 | TGGACTACTGGGACGGGAGC |
| (Chothia) | | TTAT |
| BAP049-hum08 LC | | |
| SEQ ID NO: 247 (Kabat) | LCDR1 | AAGTCCAGTCAGAGTCTGTT |
| | | AGACAGTGGAAATCAAAAGAACTT |
| | | CTTGACC |
| SEQ ID NO: 248 (Kabat) | LCDR2 | TGGGCATCCACTAGGGAATC |
| | | T |
| SEQ ID NO: 253 (Kabat) | LCDR3 | CAGAATGATTATAGTTATCC |
| | | GTACACG |
| SEQ ID NO: 250 | LCDR1 | AGTCAGAGTCTGTTAGACAG |
| (Chothia) | | TGGAAATCAAAAGAACTTC |
| SEQ ID NO: 251 | LCDR2 | TGGGCATCC |
| (Chothia) | | |
| SEQ ID NO: 254 | LCDR3 | GATTATAGTTATCCGTAC |
| (Chothia) | | |
| BAP049-hum09 HC | | |
| SEQ ID NO: 242 (Kabat) | HCDR1 | ACTTACTGGATGCAC |
| SEQ ID NO: 243 (Kabat) | HCDR2 | AATATTTATCCTGGTACTGG |
| | | TGGTTCTAACTTCGATGAGAAGTTC |
| | | AAGAAC |
| SEQ ID NO: 244 (Kabat) | HCDR3 | TGGACTACTGGGACGGGAGC |
| | | TTAT |
| SEQ ID NO: 245 | HCDR1 | GGCTACACATTCACCACTTA |
| (Chothia) | | C |
| SEQ ID NO: 246 | HCDR2 | TATCCTGGTACTGGTGGT |
| (Chothia) | | |
| SEQ ID NO: 244 | HCDR3 | TGGACTACTGGGACGGGAGC |
| (Chothia) | | TTAT |
| BAP049-hum09 LC | | |
| SEQ ID NO: 247 (Kabat) | LCDR1 | AAGTCCAGTCAGAGTCTGTT |
| | | AGACAGTGGAAATCAAAAGAACTT |
| | | CTTGACC |
| SEQ ID NO: 248 (Kabat) | LCDR2 | TGGGCATCCACTAGGGAATC |
| | | T |
| SEQ ID NO: 253 (Kabat) | LCDR3 | CAGAATGATTATAGTTATCC |
| | | GTACACG |
| SEQ ID NO: 250 | LCDR1 | AGTCAGAGTCTGTTAGACAG |
| (Chothia) | | TGGAAATCAAAAGAACTTC |
| SEQ ID NO: 251 | LCDR2 | TGGGCATCC |
| (Chothia) | | |
| SEQ ID NO: 254 | LCDR3 | GATTATAGTTATCCGTAC |
| (Chothia) | | |
| BAP049-hum10 HC | | |
| SEQ ID NO: 242 (Kabat) | HCDR1 | ACTTACTGGATGCAC |
| SEQ ID NO: 243 (Kabat) | HCDR2 | AATATTTATCCTGGTACTGG |
| | | TGGTTCTAACTTCGATGAGAAGTTC |
| | | AAGAAC |
| SEQ ID NO: 244 (Kabat) | HCDR3 | TGGACTACTGGGACGGGAGC |
| | | TTAT |
| SEQ ID NO: 245 | HCDR1 | GGCTACACATTCACCACTTA |
| (Chothia) | | C |
| SEQ ID NO: 246 | HCDR2 | TATCCTGGTACTGGTGGT |
| (Chothia) | | |
| SEQ ID NO: 244 | HCDR3 | TGGACTACTGGGACGGGAGC |
| (Chothia) | | TTAT |
| BAP049-hum10 LC | | |
| SEQ ID NO: 247 (Kabat) | LCDR1 | AAGTCCAGTCAGAGTCTGTT |
| | | AGACAGTGGAAATCAAAAGAACTT |
| | | CTTGACC |
| SEQ ID NO: 248 (Kabat) | LCDR2 | TGGGCATCCACTAGGGAATC |
| | | T |
| SEQ ID NO: 253 (Kabat) | LCDR3 | CAGAATGATTATAGTTATCC |
| | | GTACACG |
| SEQ ID NO: 250 | LCDR1 | AGTCAGAGTCTGTTAGACAG |
| (Chothia) | | TGGAAATCAAAAGAACTTC |
| SEQ ID NO: 251 | LCDR2 | TGGGCATCC |
| (Chothia) | | |
| SEQ ID NO: 254 | LCDR3 | GATTATAGTTATCCGTAC |
| (Chothia) | | |
| BAP049-hum11 HC | | |
| SEQ ID NO: 242 (Kabat) | HCDR1 | ACTTACTGGATGCAC |
| SEQ ID NO: 243 (Kabat) | HCDR2 | AATATTTATCCTGGTACTGG |
| | | TGGTTCTAACTTCGATGAGAAGTTC |
| | | AAGAAC |
| SEQ ID NO: 244 (Kabat) | HCDR3 | TGGACTACTGGGACGGGAGC |
| | | TTAT |
| SEQ ID NO: 245 | HCDR1 | GGCTACACATTCACCACTTA |
| (Chothia) | | C |
| SEQ ID NO: 246 | HCDR2 | TATCCTGGTACTGGTGGT |
| (Chothia) | | |
| SEQ ID NO: 244 | HCDR3 | TGGACTACTGGGACGGGAGC |
| (Chothia) | | TTAT |
| BAP049-hum11 LC | | |
| SEQ ID NO: 247 (Kabat) | LCDR1 | AAGTCCAGTCAGAGTCTGTT |
| | | AGACAGTGGAAATCAAAAGAACTT |
| | | CTTGACC |
| SEQ ID NO: 248 (Kabat) | LCDR2 | TGGGCATCCACTAGGGAATC |
| | | T |
| SEQ ID NO: 253 (Kabat) | LCDR3 | CAGAATGATTATAGTTATCC |
| | | GTACACG |
| SEQ ID NO: 250 | LCDR1 | AGTCAGAGTCTGTTAGACAG |
| (Chothia) | | TGGAAATCAAAAGAACTTC |
| SEQ ID NO: 251 | LCDR2 | TGGGCATCC |
| (Chothia) | | |
| SEQ ID NO: 254 | LCDR3 | GATTATAGTTATCCGTAC |
| (Chothia) | | |
| BAP049-hum12 HC | | |
| SEQ ID NO: 242 (Kabat) | HCDR1 | ACTTACTGGATGCAC |
| SEQ ID NO: 243 (Kabat) | HCDR2 | AATATTTATCCTGGTACTGG |
| | | TGGTTCTAACTTCGATGAGAAGTTC |
| | | AAGAAC |
| SEQ ID NO: 244 (Kabat) | HCDR3 | TGGACTACTGGGACGGGAGC |
| | | TTAT |
| SEQ ID NO: 245 | HCDR1 | GGCTACACATTCACCACTTA |
| (Chothia) | | C |
| SEQ ID NO: 246 | HCDR2 | TATCCTGGTACTGGTGGT |
| (Chothia) | | |
| SEQ ID NO: 244 | HCDR3 | TGGACTACTGGGACGGGAGC |
| (Chothia) | | TTAT |
| BAP049-hum12 LC | | |
| SEQ ID NO: 247 (Kabat) | LCDR1 | AAGTCCAGTCAGAGTCTGTT |
| | | AGACAGTGGAAATCAAAAGAACTT |
| | | CTTGACC |
| SEQ ID NO: 248 (Kabat) | LCDR2 | TGGGCATCCACTAGGGAATC |
| | | T |
| SEQ ID NO: 253 (Kabat) | LCDR3 | CAGAATGATTATAGTTATCC |
| | | GTACACG |
| SEQ ID NO: 250 | LCDR1 | AGTCAGAGTCTGTTAGACAG |
| (Chothia) | | TGGAAATCAAAAGAACTTC |
| SEQ ID NO: 251 | LCDR2 | TGGGCATCC |
| (Chothia) | | |
| SEQ ID NO: 254 | LCDR3 | GATTATAGTTATCCGTAC |
| (Chothia) | | |
| BAP049-hum13 HC | | |
| SEQ ID NO: 242 (Kabat) | HCDR1 | ACTTACTGGATGCAC |
| SEQ ID NO: 243 (Kabat) | HCDR2 | AATATTTATCCTGGTACTGG |
| | | TGGTTCTAACTTCGATGAGAAGTTC |
| | | AAGAAC |
| SEQ ID NO: 244 (Kabat) | HCDR3 | TGGACTACTGGGACGGGAGC |
| | | TTAT |
| SEQ ID NO: 245 | HCDR1 | GGCTACACATTCACCACTTA |
| (Chothia) | | C |
| SEQ ID NO: 246 | HCDR2 | TATCCTGGTACTGGTGGT |
| (Chothia) | | |
| SEQ ID NO: 244 | HCDR3 | TGGACTACTGGGACGGGAGC |
| (Chothia) | | TTAT |
| BAP049-hum13 LC | | |
| SEQ ID NO: 285 (Kabat) | LCDR1 | AAGTCCAGTCAGAGTCTGTT |
| | | AGACAGTGGAAATCAAAAGAACTT |
| | | CTTAACC |
| SEQ ID NO: 248 (Kabat) | LCDR2 | TGGGCATCCACTAGGGAATC |
| | | T |
| SEQ ID NO: 253 (Kabat) | LCDR3 | CAGAATGATTATAGTTATCC |
| | | GTACACG |
| SEQ ID NO: 250 | LCDR1 | AGTCAGAGTCTGTTAGACAG |
| (Chothia) | | TGGAAATCAAAAGAACTTC |
| SEQ ID NO: 251 | LCDR2 | TGGGCATCC |
| (Chothia) | | |
| SEQ ID NO: 254 | LCDR3 | GATTATAGTTATCCGTAC |
| (Chothia) | | |
| BAP049-hum14 HC | | |
| SEQ ID NO: 242 (Kabat) | HCDR1 | ACTTACTGGATGCAC |
| SEQ ID NO: 243 (Kabat) | HCDR2 | AATATTTATCCTGGTACTGG |
| | | TGGTTCTAACTTCGATGAGAAGTTC |
| | | AAGAAC |
| SEQ ID NO: 255 (Kabat) | HCDR3 | TGGACTACTGGGACGGGAGC |
| | | TTAc |
| SEQ ID NO: 245 | HCDR1 | GGCTACACATTCACCACTTA |
| (Chothia) | | |
| SEQ ID NO: 246 | HCDR2 | TATCCTGGTACTGGTGGT |
| (Chothia) | | |
| SEQ ID NO: 255 | HCDR3 | TGGACTACTGGGACGGGAGC |
| (Chothia) | | TTAc |
| BAP049-hum14 LC | | |
| SEQ ID NO: 247 (Kabat) | LCDR1 | AAGTCCAGTCAGAGTCTGTT |
| | | AGACAGTGGAAATCAAAAGAACTT |
| | | CTTGACC |
| SEQ ID NO: 248 (Kabat) | LCDR2 | TGGGCATCCACTAGGGAATC |
| | | T |
| SEQ ID NO: 253 (Kabat) | LCDR3 | CAGAATGATTATAGTTATCC |
| | | GTACACG |
| SEQ ID NO: 250 | LCDR1 | AGTCAGAGTCTGTTAGACAG |
| (Chothia) | | TGGAAATCAAAAGAACTTC |
| SEQ ID NO: 251 | LCDR2 | TGGGCATCC |
| (Chothia) | | |
| SEQ ID NO: 254 | LCDR3 | GATTATAGTTATCCGTAC |
| (Chothia) | | |
| BAP049-hum15 HC | | |
| SEQ ID NO: 242 (Kabat) | HCDR1 | ACTTACTGGATGCAC |
| SEQ ID NO: 243 (Kabat) | HCDR2 | AATATTTATCCTGGTACTGG |
| | | TGGTTCTAACTTCGATGAGAAGTTC |
| | | AAGAAC |
| SEQ ID NO: 255 (Kabat) | HCDR3 | TGGACTACTGGGACGGGAGC |
| | | TTAc |
| SEQ ID NO: 245 | HCDR1 | GGCTACACATTCACCACTTA |
| (Chothia) | | C |
| SEQ ID NO: 246 | HCDR2 | TATCCTGGTACTGGTGGT |
| (Chothia) | | |
| SEQ ID NO: 255 | HCDR3 | TGGACTACTGGGACGGGAGC |
| (Chothia) | | TTAc |
| BAP049-hum15 LC | | |
| SEQ ID NO: 247 (Kabat) | LCDR1 | AAGTCCAGTCAGAGTCTGTT |
| | | AGACAGTGGAAATCAAAAGAACTT |
| | | CTTGACC |
| SEQ ID NO: 248 (Kabat) | LCDR2 | TGGGCATCCACTAGGGAATC |
| | | T |
| SEQ ID NO: 253 (Kabat) | LCDR3 | CAGAATGATTATAGTTATCC |
| | | GTACACG |
| SEQ ID NO: 250 | LCDR1 | AGTCAGAGTCTGTTAGACAG |
| (Chothia) | | TGGAAATCAAAAGAACTTC |
| SEQ ID NO: 251 | LCDR2 | TGGGCATCC |
| (Chothia) | | |
| SEQ ID NO: 254 | LCDR3 | GATTATAGTTATCCGTAC |
| (Chothia) | | |
| BAP049-hum16 HC | | |
| SEQ ID NO: 242 (Kabat) | HCDR1 | ACTTACTGGATGCAC |
| SEQ ID NO: 243 (Kabat) | HCDR2 | AATATTTATCCTGGTACTGG |
| | | TGGTTCTAACTTCGATGAGAAGTTC |
| | | AAGAAC |
| SEQ ID NO: 244 (Kabat) | HCDR3 | TGGACTACTGGGACGGGAGC |
| | | TTAT |
| SEQ ID NO: 245 | HCDR1 | GGCTACACATTCACCACTTA |
| (Chothia) | | C |
| SEQ ID NO: 246 | HCDR2 | TATCCTGGTACTGGTGGT |
| (Chothia) | | |
| SEQ ID NO: 244 | HCDR3 | TGGACTACTGGGACGGGAGC |
| (Chothia) | | TTAT |
| BAP049-hum16 LC | | |
| SEQ ID NO: 247 (Kabat) | LCDR1 | AAGTCCAGTCAGAGTCTGTT |
| | | AGACAGTGGAAATCAAAAGAACTT |
| | | CTTGACC |
| SEQ ID NO: 248 (Kabat) | LCDR2 | TGGGCATCCACTAGGGAATC |
| | | T |
| SEQ ID NO: 253 (Kabat) | LCDR3 | CAGAATGATTATAGTTATCC |
| | | GTACACG |
| SEQ ID NO: 250 | LCDR1 | AGTCAGAGTCTGTTAGACAG |
| (Chothia) | | TGGAAATCAAAAGAACTTC |
| SEQ ID NO: 251 | LCDR2 | TGGGCATCC |
| (Chothia) | | |
| SEQ ID NO: 254 | LCDR3 | |
| (Chothia) | | GATTATAGTTATCCGTAC |
| BAP049-Clone-A HC | | |
| SEQ ID NO: 256 (Kabat) | HCDR1 | ACCTACTGGATGCAC |
| SEQ ID NO: 257 (Kabat) | HCDR2 | AACATCTATCCTGGCACCGG |
| | | CGGCTCCAACTTCGACGAGAAGTT |
| | | CAAGAAC |
| SEQ ID NO: 258 (Kabat) | HCDR3 | TGGACAACCGGCACAGGCGC |
| | | TTAT |
| SEQ ID NO: 259 | HCDR1 | GGCTACACCTTCACCACCTA |
| (Chothia) | | C |
| SEQ ID NO: 260 | HCDR2 | TATCCTGGCACCGGCGGC |
| (Chothia) | | |
| SEQ ID NO: 258 | HCDR3 | TGGACAACCGGCACAGGCGC |
| (Chothia) | | TTAT |
| BAP049-Clone-A LC | | |
| SEQ ID NO: 261 (Kabat) | LCDR1 | AAGTCCTCCCAGTCCCTGCT |
| | | GGACTCCGGCAACCAGAAGAACTT |
| | | CCTGACC |
| SEQ ID NO: 262 (Kabat) | LCDR2 | TGGGCCTCCACCCGGGAATC |
| | | T |
| SEQ ID NO: 263 (Kabat) | LCDR3 | CAGAACGACTACTCCTACCC |
| | | CTACACC |
| SEQ ID NO: 264 | LCDR1 | TCCCAGTCCCTGCTGGACTC |
| (Chothia) | | CGGCAACCAGAAGAACTTC |
| SEQ ID NO: 265 | LCDR2 | TGGGCCTCC |
| (Chothia) | | |
| SEQ ID NO: 266 | LCDR3 | GACTACTCCTACCCCTAC |
| (Chothia) | | |
| BAP049-Clone-B HC | | |
| SEQ ID NO: 267 (Kabat) | HCDR1 | ACCTACTGGATGCAC |
| SEQ ID NO: 268 (Kabat) | HCDR2 | AATATCTACCCCGGCACCGG |
| | | CGGCTCTAACTTCGACGAGAAGTTT |
| | | AAGAAT |
| SEQ ID NO: 269 (Kabat) | HCDR3 | TGGACTACCGGCACAGGCGC |
| | | CTAC |
| SEQ ID NO: 270 | HCDR1 | GGCTACACCTTCACTACCTA |
| (Chothia) | | C |
| SEQ ID NO: 271 | HCDR2 | TACCCCGGCACCGGCGGC |
| (Chothia) | | |
| SEQ ID NO: 269 | HCDR3 | TGGACTACCGGCACAGGCGC |
| (Chothia) | | CTAC |
| BAP049-Clone-B LC | | |
| SEQ ID NO: 272 (Kabat) | LCDR1 | AAATCTAGTCAGTCACTGCT |
| | | GGATAGCGGTAATCAGAAGAACTT |
| | | CCTGACC |
| SEQ ID NO: 273 (Kabat) | LCDR2 | TGGGCCTCTACTAGAGAATC |
| | | A |
| SEQ ID NO: 274 (Kabat) | LCDR3 | CAGAACGACTATAGCTACCC |
| | | CTACACC |
| SEQ ID NO: 275 | LCDR1 | AGTCAGTCACTGCTGGATAG |
| (Chothia) | | |
| SEQ ID NO: 276 | LCDR2 | CGGTAATCAGAAGAACTTC |
| (Chothia) | | TGGGCCTCT |
| SEQ ID NO: 277 | LCDR3 | GACTATAGCTACCCCTAC |
| (Chothia) | | |
| BAP049-Clone-C HC | | |
| SEQ ID NO: 256 (Kabat) | HCDR1 | ACCTACTGGATGCAC |
| SEQ ID NO: 257 (Kabat) | HCDR2 | AACATCTATCCTGGCACCGG |
| | | CGGCTCCAACTTCGACGAGAAGTT |
| | | CAAGAAC |
| SEQ ID NO: 258 (Kabat) | HCDR3 | TGGACAACCGGCACAGGCGC |
| | | TTAT |
| SEQ ID NO: 259 | HCDR1 | GGCTACACCTTCACCACCTA |
| (Chothia) | | C |
| SEQ ID NO: 260 | HCDR2 | TATCCTGGCACCGGCGGC |
| (Chothia) | | |
| SEQ ID NO: 258 | HCDR3 | TGGACAACCGGCACAGGCGC |
| (Chothia) | | TTAT |
| BAP049-Clone-C LC | | |
| SEQ ID NO: 261 (Kabat) | LCDR1 | AAGTCCTCCCAGTCCCTGCT |
| | | GGACTCCGGCAACCAGAAGAACTT |
| | | CCTGACC |
| SEQ ID NO: 262 (Kabat) | LCDR2 | TGGGCCTCCACCCGGGAATC |
| | | T |
| SEQ ID NO: 263 (Kabat) | LCDR3 | CAGAACGACTACTCCTACCC |
| | | CTACACC |
| SEQ ID NO: 264 | LCDR1 | TCCCAGTCCCTGCTGGACTC |
| (Chothia) | | CGGCAACCAGAAGAACTTC |
| SEQ ID NO: 265 | LCDR2 | TGGGCCTCC |
| (Chothia) | | |
| SEQ ID NO: 266 | LCDR3 | GACTACTCCTACCCCTAC |
| (Chothia) | | |
| BAP049-Clone-D HC | | |
| SEQ ID NO: 256 (Kabat) | HCDR1 | ACCTACTGGATGCAC |
| SEQ ID NO: 278 (Kabat) | HCDR2 | AACATCTACCCTGGCACCGG |
| | | CGGCTCCAACTTCGACGAGAAGTT |
| | | CAAGAAC |
| SEQ ID NO: 279 (Kabat) | HCDR3 | TGGACCACCGGAACCGGCGC |
| | | CTAT |
| SEQ ID NO: 259 | HCDR1 | GGCTACACCTTCACCACCTA |
| (Chothia) | | C |
| SEQ ID NO: 280 | HCDR2 | TACCCTGGCACCGGCGGC |
| (Chothia) | | |
| SEQ ID NO: 279 | HCDR3 | TGGACCACCGGAACCGGCGC |
| (Chothia) | | CTAT |
| BAP049-Clone-D LC | | |
| SEQ ID NO: 261 (Kabat) | LCDR1 | AAGTCCTCCCAGTCCCTGCT |
| | | GGACTCCGGCAACCAGAAGAACTT |
| | | CCTGACC |
| SEQ ID NO: 262 (Kabat) | LCDR2 | TGGGCCTCCACCCGGGAATC |
| | | T |
| SEQ ID NO: 263 (Kabat) | LCDR3 | CAGAACGACTACTCCTACCC |
| | | CTACACC |
| SEQ ID NO: 264 | LCDR1 | TCCCAGTCCCTGCTGGACTC |
| (Chothia) | | CGGCAACCAGAAGAACTTC |
| SEQ ID NO: 265 | LCDR2 | TGGGCCTCC |
| (Chothia) | | |
| SEQ ID NO: 266 | LCDR3 | GACTACTCCTACCCCTAC |
| (Chothia) | | |
| BAP049-Clone-E HC | | |
| SEQ ID NO: 267 (Kabat) | HCDR1 | ACCTACTGGATGCAC |
| SEQ ID NO: 268 (Kabat) | HCDR2 | AATATCTACCCCGGCACCGG |
| | | CGGCTCTAACTTCGACGAGAAGTTT |
| | | AAGAAT |
| SEQ ID NO: 269 (Kabat) | HCDR3 | TGGACTACCGGCACAGGCGC |
| | | CTAC |
| SEQ ID NO: 270 | HCDR1 | GGCTACACCTTCACTACCTA |
| (Chothia) | | C |
| SEQ ID NO: 271 | HCDR2 | TACCCCGGCACCGGCGGC |
| (Chothia) | | |
| SEQ ID NO: 269 | HCDR3 | TGGACTACCGGCACAGGCGC |
| (Chothia) | | CTAC |
| BAP049-Clone-E LC | | |
| SEQ ID NO: 272 (Kabat) | LCDR1 | AAATCTAGTCAGTCACTGCT |
| | | GGATAGCGGTAATCAGAAGAACTT |
| | | CCTGACC |
| SEQ ID NO: 273 (Kabat) | LCDR2 | TGGGCCTCTACTAGAGAATC |
| | | A |
| SEQ ID NO: 274 (Kabat) | LCDR3 | CAGAACGACTATAGCTACCC |
| | | CTACACC |
| SEQ ID NO: 275 | LCDR1 | AGTCAGTCACTGCTGGATAG |
| (Chothia) | | CGGTAATCAGAAGAACTTC |
| SEQ ID NO: 276 | LCDR2 | TGGGCCTCT |
| (Chothia) | | |
| SEQ ID NO: 277 | LCDR3 | GACTATAGCTACCCCTAC |
| (Chothia) | | |
| BAP049_VH | | |
| SEQ ID NO: 533 | HCDR1 | GYTFTTYW |
| (IMGT) | | |
| SEQ ID NO: 534 | HCDR2 | IYPGTGGS |
| (IMGT) | | |
| SEQ ID NO: 535 | HCDR3 | TRWTTGTGAY |
| (IMGT) | | |
| SEQ ID NO: 142 | VH | QVQLQQPGSELVRPGASVKLS |
| | | CKASGYTFTTYWMHWVRQRPGQGL |
| | | EWIGNIYPGTGGSNFDEKFKNRTSLT |
| | | VDTSSTTAYMHLASLTSEDSAVYYC |
| | | TRWTTGTGAYWGQGTLVTVSA |
| BAP049_VL | | |
| SEQ ID NO: 536 | LCDR1 | QSLLDSGNQKNF |
| (IMGT) | | |
| SEQ ID NO: 150 | LCDR2 | WAS |
| (IMGT) | | |
| SEQ ID NO: 148 | LCDR3 | QNDYSYPCT |
| (IMGT) | | |
| SEQ ID NO: 152 | VL | SCKSSQSLLDSGNQKNFLTWYQQKP |
| | | GQPPKLLIFWASTRESGVPDRFTGSG |
| | | SVTDFTLTISSVQAEDLAVYYCQND |
| | | YSYPCTFGGGTKLEIK |
| BAP049-chi_v1_HC | | |
| SEQ ID NO: 533 | HCDR1 | GYTFTTYW |
| (IMGT) | | |
| SEQ ID NO: 534 | HCDR2 | IYPGTGGS |
| (IMGT) | | |
| SEQ ID NO: 535 | HCDR3 | TRWTTGTGAY |
| (IMGT) | | |
| SEQ ID NO: 154 | VH | QVQLQQPGSELVRPGASVKLS |
| | | CKASGYTFTTYWMHWVRQRPGQGL |
| | | EWIGNIYPGTGGSNFDEKFKNRTSLT |
| | | VDTSSTTAYMHLASLTSEDSAVYYC |
| | | TRWTTGTGAYWGQGTTVTVSS |
| BAP049_chi_v2_HC | | |
| SEQ ID NO: 533 | HCDR1 | GYTFTTYW |
| (IMGT) | | |
| SEQ ID NO: 534 | HCDR2 | IYPGTGGS |
| (IMGT) | | |
| SEQ ID NO: 535 | HCDR3 | TRWTTGTGAY |
| (IMGT) | | |
| SEQ ID NO: 158 | VH | QVQLQQSGSELVRPGASVKLS |
| | | CKASGYTFTTYWMHWVRQRPGQGL |
| | | EWIGNIYPGTGGSNFDEKFKNRTSLT |
| | | VDTSSTTAYMHLASLTSEDSAVYYC |
| | | TRWTTGTGAYWGQGTTVTVSS |
| BAP049_chi_LC | | |
| SEQ ID NO: 536 | LCDR1 | QSLLDSGNQKNF |
| (IMGT) | | |
| SEQ ID NO: 150 | LCDR2 | WAS |
| (IMGT) | | |
| SEQ ID NO: 148 | LCDR3 | QNDYSYPCT |
| (IMGT) | | |
| SEQ ID NO: 162 | VL | DIVMTQSPSSLTVTAGEKVTM |
| | | SCKSSQSLLDSGNQKNFLTWYQQKP |
| | | GQPPKLLIFWASTRESGVPDRFTGSG |
| | | SVTDFTLTISSVQAEDLAVYYCQND |
| | | YSYPCTFGQGTKVEIK |
| SEQ ID NO: 163 | DNA VL | GACATTGTGATGACCCAGTC |
| | | TCCATCCTCCCTGACTGTGACAGCA |
| | | GGAGAGAAGGTCACTATGAGCTGC |
| | | AAGTCCAGTCAGAGTCTGTTAGAC |
| | | AGTGGAAATCAAAAGAACTTCTTG |
| | | ACCTGGTACCAGCAGAAACCAGGG |
| | | CAGCCTCCTAAACTGTTGATCTTCT |
| | | GGGCATCCACTAGGGAATCTGGGG |
| | | TCCCTGATCGCTTCACAGGCAGTGG |
| | | ATCTGTAACAGATTTCACTCTCACC |
| | | ATCAGCAGTGTGCAGGCTGAAGAC |
| | | CTGGCAGTTTATTACTGTCAGAATG |
| | | ATTATAGTTATCCGTGCACGTTCGG |
| | | CCAAGGGACCAAGGTGGAAATCAA |
| | | A |
| BAP049-chi-Y_v1_HC | | |
| SEQ ID NO: 533 | HCDR1 | GYTFTTYW |
| (IMGT) | | |
| SEQ ID NO: 534 | HCDR2 | IYPGTGGS |
| (IMGT) | | |
| SEQ ID NO: 535 | HCDR3 | TRWTTGTGAY |
| (IMGT) | | |
| SEQ ID NO: 154 | VH | QVQLQQPGSELVRPGASVKLS |
| | | CKASGYTFTTYWMHWVRQRPGQGL |
| | | EWIGNIYPGTGGSNFDEKFKNRTSLT |
| | | VDTSSTTAYMHLASLTSEDSAVYYC |
| | | TRWTTGTGAYWGQGTTVTVSS |
| BAP049-chi-Y_v2_HC | | |
| SEQ ID NO: 533 | HCDR1 | GYTFTTYW |
| (IMGT) | | |
| SEQ ID NO: 534 | HCDR2 | IYPGTGGS |
| (IMGT) | | |
| SEQ ID NO: 535 | HCDR3 | TRWTTGTGAY |
| (IMGT) | | |
| SEQ ID NO: 158 | VH | QVQLQQSGSELVRPGASVKLS |
| | | CKASGYTFTTYWMHWVRQRPGQGL |
| | | EWIGNIYPGTGGSNFDEKFKNRTSLT |
| | | VDTSSTTAYMHLASLTSEDSAVYYC |
| | | TRWTTGTGAYWGQGTTVTVSS |
| BAP049-chi-Y_LC | | |
| SEQ ID NO: 536 | LCDR1 | QSLLDSGNQKNF |
| (IMGT) | | |
| SEQ ID NO: 150 | LCDR2 | WAS |
| (IMGT) | | |
| SEQ ID NO: 166 | LCDR3 | QNDYSYPYT |
| (IMGT) | | |
| SEQ ID NO: 168 | VL | DIVMTQSPSSLTVTAGEKVTM |
| | | SCKSSQSLLDSGNQKNFLTWYQQKP |
| | | GQPPKLLIFWASTRESGVPDRFTGSG |
| | | SVTDFTLTISSVQAEDLAVYYCQND |
| | | YSYPYTFGQGTKVEIK |
| BAP049-hum01_HC | | |
| SEQ ID NO: 533 | HCDR1 | GYTFTTYW |
| (IMGT) | | |
| SEQ ID NO: 534 | HCDR2 | IYPGTGGS |
| (IMGT) | | |
| SEQ ID NO: 535 | HCDR3 | TRWTTGTGAY |
| (IMGT) | | |
| SEQ ID NO: 172 | VH | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSS |
| BAP049-hum01_LC | | |
| SEQ ID NO: 536 | LCDR1 | QSLLDSGNQKNF |
| (IMGT) | | |
| SEQ ID NO: 150 | LCDR2 | WAS |
| (IMGT) | | |
| SEQ ID NO: 166 | LCDR3 | QNDYSYPYT |
| (IMGT) | | |
| SEQ ID NO: 176 | VL | EIVLTQSPATLSLSPGERATLS |
| | | CKSSQSLLDSGNQKNFLTWYQQKPG |
| | | QAPRLLIYWASTRESGVPSRFSGSGS |
| | | GTEFTLTISSLQPDDFATYYCQNDYS |
| | | YPYTFGQGTKVEIK |
| BAP049-hum02_HC | | |
| SEQ ID NO: 533 | HCDR1 | GYTFTTYW |
| (IMGT) | | |
| SEQ ID NO: 534 | HCDR2 | IYPGTGGS |
| (IMGT) | | |
| SEQ ID NO: 535 | HCDR3 | TRWTTGTGAY |
| (IMGT) | | |
| SEQ ID NO: 172 | VH | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSS |
| BAP049-hum02_LC | | |
| SEQ ID NO: 536 | LCDR1 | QSLLDSGNQKNF |
| (IMGT) | | |
| SEQ ID NO: 150 | LCDR2 | WAS |
| (IMGT) | | |
| SEQ ID NO: 166 | LCDR3 | QNDYSYPYT |
| (IMGT) | | |
| SEQ ID NO: 180 | VL | DIQMTQSPSSLSASVGDRVTIT |
| | | CKSSQSLLDSGNQKNFLTWYQQKPG |
| | | QAPRLLIYWASTRESGIPPRFSGSGY |
| | | GTDFTLTINNIESEDAAYYFCQNDYS |
| | | YPYTFGQGTKVEIK |
| BAP049-hum03_HC | | |
| SEQ ID NO: 533 | HCDR1 | GYTFTTYW |
| (IMGT) | | |
| SEQ ID NO: 534 | HCDR2 | IYPGTGGS |
| (IMGT) | | |
| SEQ ID NO: 535 | HCDR3 | TRWTTGTGAY |
| (IMGT) | | |
| SEQ ID NO: 184 | VH | EVQLVQSGAEVKKPGESLRIS |
| | | CKGSGYTFTTYWMHWIRQSPSRGLE |
| | | WLGNIYPGTGGSNFDEKFKNRFTISR |
| | | DNSKNTLYLQMNSLRAEDTAVYYC |
| | | TRWTTGTGAYWGQGTTVTVSS |
| BAP049-hum03_LC | | |
| SEQ ID NO: 536 | LCDR1 | QSLLDSGNQKNF |
| (IMGT) | | |
| SEQ ID NO: 150 | LCDR2 | WAS |
| (IMGT) | | |
| SEQ ID NO: 166 | LCDR3 | QNDYSYPYT |
| (IMGT) | | |
| SEQ ID NO: 180 | VL | DIQMTQSPSSLSASVGDRVTIT |
| | | CKSSQSLLDSGNQKNFLTWYQQKPG |
| | | QAPRLLIYWASTRESGIPPRFSGSGY |
| | | GTDFTLTINNIESEDAAYYFCQNDYS |
| | | YPYTFGQGTKVEIK |
| BAP049-hum04_HC | | |
| SEQ ID NO: 533 | HCDR1 | GYTFTTYW |
| (IMGT) | | |
| SEQ ID NO: 534 | HCDR2 | IYPGTGGS |
| (IMGT) | | |
| SEQ ID NO: 535 | HCDR3 | TRWTTGTGAY |
| (IMGT) | | |
| SEQ ID NO: 184 | VH | EVQLVQSGAEVKKPGESLRIS |
| | | CKGSGYTFTTYWMHWIRQSPSRGLE |
| | | WLGNIYPGTGGSNFDEKFKNRFTISR |
| | | DNSKNTLYLQMNSLRAEDTAVYYC |
| | | TRWTTGTGAYWGQGTTVTVSS |
| BAP049-hum04_LC | | |
| SEQ ID NO: 536 | LCDR1 | QSLLDSGNQKNF |
| (IMGT) | | |
| SEQ ID NO: 150 | LCDR2 | WAS |
| (IMGT) | | |
| SEQ ID NO: 166 | LCDR3 | QNDYSYPYT |
| (IMGT) | | |
| SEQ ID NO: 188 | VL | EIVLTQSPATLSLSPGERATLS |
| | | CKSSQSLLDSGNQKNFLTWYQQKPG |
| | | KAPKLLIYWASTRESGVPSRFSGSGS |
| | | GTDFTFTISSLQPEDIATYYCQNDYS |
| | | YPYTFGQGTKVEIK |
| BAP049-hum05_HC | | |
| SEQ ID NO: 533 | HCDR1 | GYTFTTYW |
| (IMGT) | | |
| SEQ ID NO: 534 | HCDR2 | IYPGTGGS |
| (IMGT) | | |
| SEQ ID NO: 535 | HCDR3 | TRWTTGTGAY |
| (IMGT) | | |
| SEQ ID NO: 172 | VH | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSS |
| BAP049-hum05_LC | | |
| SEQ ID NO: 536 | LCDR1 | QSLLDSGNQKNF |
| (IMGT) | | |
| SEQ ID NO: 150 | LCDR2 | WAS |
| (IMGT) | | |
| SEQ ID NO: 166 | LCDR3 | QNDYSYPYT |
| (IMGT) | | |
| SEQ ID NO: 188 | VL | EIVLTQSPATLSLSPGERATLS |
| | | CKSSQSLLDSGNQKNFLTWYQQKPG |
| | | KAPKLLIYWASTRESGVPSRFSGSGS |
| | | GTDFTFTISSLQPEDIATYYCQNDYS |
| | | YPYTFGQGTKVEIK |
| BAP049-hum06_HC | | |
| SEQ ID NO: 533 | HCDR1 | GYTFTTYW |
| (IMGT) | | |
| SEQ ID NO: 534 | HCDR2 | IYPGTGGS |
| (IMGT) | | |
| SEQ ID NO: 535 | HCDR3 | TRWTTGTGAY |
| (IMGT) | | |
| SEQ ID NO: 172 | VH | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSS |
| BAP049-hum06_LC | | |
| SEQ ID NO: 536 | LCDR1 | QSLLDSGNQKNF |
| (IMGT) | | |
| SEQ ID NO: 150 | LCDR2 | WAS |
| (IMGT) | | |
| SEQ ID NO: 166 | LCDR3 | QNDYSYPYT |
| (IMGT) | | |
| SEQ ID NO: 192 | VL | DIVMTQTPLSLPVTPGEPASIS |
| | | CKSSQSLLDSGNQKNFLTWYQQKPG |
| | | QAPRLLIYWASTRESGVPSRFSGSGS |
| | | GTDFTFTISSLEAEDAATYYCQNDYS |
| | | YPYTFGQGTKVEIK |
| BAP049-hum07_HC | | |
| SEQ ID NO: 533 | HCDR1 | GYTFTTYW |
| (IMGT) | | |
| SEQ ID NO: 534 | HCDR2 | IYPGTGGS |
| (IMGT) | | |
| SEQ ID NO: 535 | HCDR3 | TRWTTGTGAY |
| (IMGT) | | |
| SEQ ID NO: 172 | VH | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSS |
| BAP049-hum07_LC | | |
| SEQ ID NO: 536 | LCDR1 | QSLLDSGNQKNF |
| (IMGT) | | |
| SEQ ID NO: 150 | LCDR2 | WAS |
| (IMGT) | | |
| SEQ ID NO: 166 | LCDR3 | QYDYSYPTY |
| (IMGT) | | |
| SEQ ID NO: 196 | VL | EIVLTQSPATLSLSPGERATLS |
| | | CKSSQSLLDSGNQKNFLTWYQQKPG |
| | | KAPKLLIYWASTRESGVPSRFSGSGS |
| | | GTDFTFTISSLEAEDAATYYCQNDYS |
| | | YPYTFGQGTKVEIK |
| BAP049-hum08_HC | | |
| SEQ ID NO: 533 | HCDR1 | GYTFTTYW |
| (IMGT) | | |
| SEQ ID NO: 534 | HCDR2 | IYPGTGGS |
| (IMGT) | | |
| SEQ ID NO: 535 | HCDR3 | TRWTTGTGAY |
| (IMGT) | | |
| SEQ ID NO: 184 | VH | EVQLVQSGAEVKKPGESLRIS |
| | | CKGSGYTFTTYWMHWIRQSPSRGLE |
| | | WLGNIYPGTGGSNFDEKFKNRFTISR |
| | | DNSKNTLYLQMNSLRAEDTAVYYC |
| | | TRWTTGTGAYWGQGTTVTVSS |
| BAP049-hum08_LC | | |
| SEQ ID NO: 36 | LCDR1 | QSLLDSGNQKNF |
| (IMGT) | | |
| SEQ ID NO: 150 | LCDR2 | WAS |
| (IMGT) | | |
| SEQ ID NO: 166 | LCDR3 | QNDYSYPYT |
| (IMGT) | | |
| SEQ ID NO: 200 | VL | EIVLTQSPDFQSVTPKEKVTIT |
| | | CKSSQSLLDSGNQKNFLTWYQQKPG |
| | | QAPRLLIYWASTRESGVPSRFSGSGS |
| | | GTDFTFTISSLEAEDAATYYCQNDYS |
| | | YPYTFGQGTKVEIK |
| BAP049-hum09_HC | | |
| SEQ ID NO: 533 | HCDR1 | GYTFTTYW |
| (IMGT) | | |
| SEQ ID NO: 534 | HCDR2 | IYPGTGGS |
| (IMGT) | | |
| SEQ ID NO: 535 | HCDR3 | TRWTTGTGAY |
| (IMGT) | | |
| SEQ ID NO: 172 | VH | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSS |
| BAP049-hum09_LC | | |
| SEQ ID NO: 536 | LCDR1 | QSLLDSGNQKNF |
| (IMGT) | | |
| SEQ ID NO: 150 | LCDR2 | WAS |
| (IMGT) | | |
| SEQ ID NO: 166 | LCDR3 | QNDYSYPYT |
| (IMGT) | | |
| SEQ ID NO: 200 | VL | EIVLTQSPDFQSVTPKEKVTIT |
| | | CKSSQSLLDSGNQKNFLTWYQQKPG |
| | | QAPRLLIYWASTRESGVPSRFSGSGS |
| | | GTDFTFTISSLEAEDAATYYCQNDYS |
| | | YPYTFGQGTKVEIK |
| BAP049-hum10_HC | | |
| SEQ ID NO: 533 | HCDR1 | GYTFTTYW |
| (IMGT) | | |
| SEQ ID NO: 534 | HCDR2 | IYPGTGGS |
| (IMGT) | | |
| SEQ ID NO: 535 | HCDR3 | TRWTTGTGAY |
| (IMGT) | | |
| SEQ ID NO: 184 | VH | EVQLVQSGAEVKKPGESLRIS |
| | | CKGSGYTFTTYWMHWIRQSPSRGLE |
| | | WLGNIYPGTGGSNFDEKFKNRFTISR |
| | | DNSKNTLYLQMNSLRAEDTAVYYC |
| | | TRWTTGTGAYWGQGTTVTVSS |
| BAP049-hum10_LC | | |
| SEQ ID NO: 536 | LCDR1 | QSLLDSGNQKNF |
| (IMGT) | | |
| SEQ ID NO: 150 | LCDR2 | WAS |
| (IMGT) | | |
| SEQ ID NO: 166 | LCDR3 | QNDYSYPYT |
| (IMGT) | | |
| SEQ ID NO: 204 | VL | EIVLTQSPATLSLSPGERATLS |
| | | CKSSQSLLDSGNQKNFLTWYQQKPG |
| | | QAPRLLIYWASTRESGVPSRFSGSGS |
| | | GTDFTFTISSLEAEDAATYYCQNDYS |
| | | YPYTFGQGTKVEIK |
| BAP049-hum11_HC | | |
| SEQ ID NO: 533 | HCDR1 | GYTFTTYW |
| (IMGT) | | |
| SEQ ID NO: 534 | HCDR2 | IYPGTGGS |
| (IMGT) | | |
| SEQ ID NO: 535 | HCDR3 | TRWTTGTGAY |
| (IMGT) | | |
| SEQ ID NO: 172 | VH | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSS |
| BAP049-hum11_LC | | |
| SEQ ID NO: 536 | LCDR1 | QSLLDSGNQKNF |
| (IMGT) | | |
| SEQ ID NO: 150 | LCDR2 | WAS |
| (IMGT) | | |
| SEQ ID NO: 166 | LCDR3 | QNDYSYPYT |
| (IMGT) | | |
| SEQ ID NO: 204 | VL | EIVLTQSPATLSLSPGERATLS |
| | | CKSSQSLLDSGNQKNFLTWYQQKPG |
| | | QAPRLLIYWASTRESGVPSRFSGSGS |
| | | GTDFTFTISSLEAEDAATYYCQNDYS |
| | | YPYTFGQGTKVEIK |
| BAP049-hum12_HC | | |
| SEQ ID NO: 533 | HCDR1 | GYTFTTYW |
| (IMGT) | | |
| SEQ ID NO: 534 | HCDR2 | IYPGTGGS |
| (IMGT) | | |
| SEQ ID NO: 535 | HCDR3 | TRWTTGTGAY |
| (IMGT) | | |
| SEQ ID NO: 172 | VH | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSS |
| BAP049-hum12_LC | | |
| SEQ ID NO: 536 | LCDR1 | QSLLDSGNQKNF |
| (IMGT) | | |
| SEQ ID NO: 150 | LCDR2 | WAS |
| (IMGT) | | |
| SEQ ID NO: 166 | LCDR3 | QNDYSYPYT |
| (IMGT) | | |
| SEQ ID NO: 208 | VL | DIQMTQSPSSLSASVGDRVTIT |
| | | CKSSQSLLDSGNQKNFLTWYLQKPG |
| | | QSPQLLIYWASTRESGVPSRFSGSGS |
| | | GTDFTFTISSLEAEDAATYYCQNDYS |
| | | YPYTFGQGTKVEIK |
| BAP049-hum13_HC | | |
| SEQ ID NO: 533 | HCDR1 | GYTFTTYW |
| (IMGT) | | |
| SEQ ID NO: 534 | HCDR2 | IYPGTGGS |
| (IMGT) | | |
| SEQ ID NO: 535 | HCDR3 | TRWTTGTGAY |
| (IMGT) | | |
| SEQ ID NO: 172 | VH | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSS |
| BAP049-hum13_LC | | |
| SEQ ID NO: 536 | LCDR1 | QSLLDSGNQKNF |
| (IMGT) | | |
| SEQ ID NO: 150 | LCDR2 | WAS |
| (IMGT) | | |
| SEQ ID NO: 166 | LCDR3 | QNDYSYPYT |
| (IMGT) | | |
| SEQ ID NO: 212 | VL | DVVMTQSPLSLPVTLGQPASIS |
| | | CKSSQSLLDSGNQKNFLTWYQQKPG |
| | | KAPKLLIYWASTRESGVPSRFSGSGS |
| | | GTDFTFTISSLEAEDAATYYCQNDYS |
| | | YPYTFGQGTKVEIK |
| BAP049-hum14_HC | | |
| SEQ ID NO: 533 | HCDR1 | GYTFTTYW |
| (IMGT) | | |
| SEQ ID NO: 534 | HCDR2 | IYPGTGGS |
| (IMGT) | | |
| SEQ ID NO: 535 | HCDR3 | TRWTTGTGAY |
| (IMGT) | | |
| SEQ ID NO: 216 | VH | QVQLVQSGAEVKKPGASVKV |
| | | SCKASGYTFTTYWMHWIRQSPSRGL |
| | | EWLGNIYPGTGGSNFDEKFKNRFTIS |
| | | RDNSKNTLYLQMNSLRAEDTAVYY |
| | | CTRWTTGTGAYWGQGTTVTVSS |
| BAP049-hum14_LC | | |
| SEQ ID NO: 536 | LCDR1 | QSLLDSGNQKNF |
| (IMGT) | | |
| SEQ ID NO: 150 | LCDR2 | WAS |
| (IMGT) | | |
| SEQ ID NO: 166 | LCDR3 | QNDYSYPYT |
| (IMGT) | | |
| SEQ ID NO: 204 | VL | EIVLTQSPATLSLSPGERATLS |
| | | CKSSQSLLDSGNQKNFLTWYQQKPG |
| | | QAPRLLIYWASTRESGVPSRFSGSGS |
| | | GTDFTFTISSLEAEDAATYYCQNDYS |
| | | YPYTFGQGTKVEIK |
| BAP049-hum15_HC | | |
| SEQ ID NO: 533 | HCDR1 | GYTFTTYW |
| (IMGT) | | |
| SEQ ID NO: 534 | HCDR2 | IYPGTGGS |
| (IMGT) | | |
| SEQ ID NO: 535 | HCDR3 | TRWTTGTGAY |
| (IMGT) | | |
| SEQ ID NO: 216 | VH | QVQLVQSGAEVKKPGASVKV |
| | | SCKASGYTFTTYWMHWIRQSPSRGL |
| | | EWLGNIYPGTGGSNFDEKFKNRFTIS |
| | | RDNSKNTLYLQMNSLRAEDTAVYY |
| | | CTRWTTGTGAYWGQGTTVTVSS |
| BAP049-hum15_LC | | |
| SEQ ID NO: 536 | LCDR1 | QSLLDSGNQKNF |
| (IMGT) | | |
| SEQ ID NO: 150 | LCDR2 | WAS |
| (IMGT) | | |
| SEQ ID NO: 166 | LCDR3 | QNDYSYPYT |
| (IMGT) | | |
| SEQ ID NO: 200 | VL | EIVLTQSPDFQSVTPKEKVTIT |
| | | CKSSQSLLDSGNQKNFLTWYQQKPG |
| | | QAPRLLIYWASTRESGVPSRFSGSGS |
| | | GTDFTFTISSLEAEDAATYYCQNDYS |
| | | YPYTFGQGTKVEIK |
| BAP049-hum16_HC | | |
| SEQ ID NO: 533 | HCDR1 | GYTFTTYW |
| (IMGT) | | |
| SEQ ID NO: 534 | HCDR2 | IYPGTGGS |
| (IMGT) | | |
| SEQ ID NO: 535 | HCDR3 | TRWTTGTGAY |
| (IMGT) | | |
| SEQ ID NO: 220 | VH | EVQLVQSGAEVKKPGESLRIS |
| | | CKGSGYTFTTYWMHWVRQAPGQGL |
| | | EWMGNIYPGTGGSNFDEKFKNRFTIS |
| | | RDNSKNTLYLQMNSLRAEDTAVYY |
| | | CTRWTTGTGAYWGQGTTVTVSS |
| BAP049-hum16_LC | | |
| SEQ ID NO: 536 | LCDR1 | QSLLDSGNQKNF |
| (IMGT) | | |
| SEQ ID NO: 150 | LCDR2 | WAS |
| (IMGT) | | |
| SEQ ID NO: 166 | LCDR3 | QNDYSYPYT |
| (IMGT) | | |
| SEQ ID NO: 200 | VL | EIVLTQSPDFQSVTPKEKVTIT |
| | | CKSSQSLLDSGNQKNFLTWYQQKPG |
| | | QAPRLLIYWASTRESGVPSRFSGSGS |
| | | GTDFTFTISSLEAEDAATYYCQNDYS |
| | | YPYTFGQGTKVEIK |
| BAP049-Clone-A_HC | | |
| SEQ ID NO: 533 | HCDR1 | GYTFTTYW |
| (IMGT) | | |
| SEQ ID NO: 534 | HCDR2 | IYPGTGGS |
| (IMGT) | | |
| SEQ ID NO: 535 | HCDR3 | TRWTTGTGAY |
| (IMGT) | | |
| SEQ ID NO: 172 | VH | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSS |
| BAP049-Clone-A_LC | | |
| SEQ ID NO: 536 | LCDR1 | QSLLDSGNQKNF |
| (IMGT) | | |
| SEQ ID NO: 150 | LCDR2 | WAS |
| (IMGT) | | |
| SEQ ID NO: 166 | LCDR3 | QNDYSYPYT |
| (IMGT) | | |
| SEQ ID NO: 176 | VL | EIVLTQSPATLSLSPGERATLS |
| | | CKSSQSLLDSGNQKNFLTWYQQKPG |
| | | QAPRLLIYWASTRESGVPSRFSGSGS |
| | | GTEFTLTISSLQPDDFATYYCQNDYS |
| | | YPYTFGQGTKVEIK |
| BAP049-Clone-B_HC | | |
| SEQ ID NO: 533 | HCDR1 | GYTFTTYW |
| (IMGT) | | |
| SEQ ID NO: 534 | HCDR2 | IYPGTGGS |
| (IMGT) | | |
| SEQ ID NO: 535 | HCDR3 | TRWTTGTGAY |
| (IMGT) | | |
| SEQ ID NO: 172 | VH | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSS |
| BAP049-Clone-B_LC | | |
| SEQ ID NO: 536 | LCDR1 | QSLLDSGNQKNF |
| (IMGT) | | |
| SEQ ID NO: 150 | LCDR2 | WAS |
| (IMGT) | | |
| SEQ ID NO: 166 | LCDR3 | QNDYSYPYT |
| (IMGT) | | |
| SEQ ID NO: 188 | VL | EIVLTQSPATLSLSPGERATLS |
| | | CKSSQSLLDSGNQKNFLTWYQQKPG |
| | | KAPKLLIYWASTRESGVPSRFSGSGS |
| | | GTDFTFTISSLQPEDIATYYCQNDYS |
| | | YPYTFGQGTKVEIK |
| BAP049-Clone-C_HC | | |
| SEQ ID NO: 533 | HCDR1 | GYTFTTYW |
| (IMGT) | | |
| SEQ ID NO: 534 | HCDR2 | IYPGTGGS |
| (IMGT) | | |
| SEQ ID NO: 535 | HCDR3 | TRWTTGTGAY |
| (IMGT) | | |
| SEQ ID NO: 172 | VH | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSS |
| BAP049-Clone-C_LC | | |
| SEQ ID NO: 536 | LCDR1 | QSLLDSGNQKNF |
| (IMGT) | | |
| SEQ ID NO: 150 | LCDR2 | WAS |
| (IMGT) | | |
| SEQ ID NO: 166 | LCDR3 | QNDYSYPYT |
| (IMGT) | | |
| SEQ ID NO: 200 | VL | EIVLTQSPDFQSVTPKEKVTIT |
| | | CKSSQSLLDSGNQKNFLTWYQQKPG |
| | | QAPRLLIYWASTRESGVPSRFSGSGS |
| | | GTDFTFTISSLEAEDAATYYCQNDYS |
| | | YPYTFGQGTKVEIK |
| BAP049-Clone-D_HC | | |
| SEQ ID NO: 533 | HCDR1 | GYTFTTYW |
| (IMGT) | | |
| SEQ ID NO: 534 | HCDR2 | IYPGTGGS |
| (IMGT) | | |
| SEQ ID NO: 535 | HCDR3 | TRWTTGTGAY |
| (IMGT) | | |
| SEQ ID NO: 184 | VH | EVQLVQSGAEVKKPGESLRIS |
| | | CKGSGYTFTTYWMHWIRQSPSRGLE |
| | | WLGNIYPGTGGSNFDEKFKNRFTISR |
| | | DNSKNTLYLQMNSLRAEDTAVYYC |
| | | TRWTTGTGAYWGQGTTVTVSS |
| BAP049-Clone-D_LC | | |
| SEQ ID NO: 536 | LCDR1 | QSLLDSGNQKNF |
| (IMGT) | | |
| SEQ ID NO: 150 | LCDR2 | WAS |
| (IMGT) | | |
| SEQ ID NO: 166 | LCDR3 | QNDYSYPYT |
| (IMGT) | | |
| SEQ ID NO: 204 | VL | EIVLTQSPATLSLSPGERATLS |
| | | CKSSQSLLDSGNQKNFLTWYQQKPG |
| | | QAPRLLIYWASTRESGVPSRFSGSGS |
| | | GTDFTFTISSLEAEDAATYYCQNDYS |
| | | YPYTFGQGTKVEIK |
| BAP049-Clone-E_HC | | |
| SEQ ID NO: 533 | HCDR1 | GYTFTTYW |
| (IMGT) | | |
| SEQ ID NO: 534 | HCDR2 | IYPGTGGS |
| (IMGT) | | |
| SEQ ID NO: 535 | HCDR3 | TRWTTGTGAY |
| (IMGT) | | |
| SEQ ID NO: 172 | VH | EVQLVQSGAEVKKPGESLRIS |
| | | CKGSGYTFTTYWMHWVRQATGQGL |
| | | EWMGNIYPGTGGSNFDEKFKNRVTI |
| | | TADKSTSTAYMELSSLRSEDTAVYY |
| | | CTRWTTGTGAYWGQGTTVTVSS |
| BAP049-Clone-E_LC | | |
| SEQ ID NO: 536 | LCDR1 | QSLLDSGNQKNF |
| (IMGT) | | |
| SEQ ID NO: 150 | LCDR2 | WAS |
| (IMGT) | | |
| SEQ ID NO: 166 | LCDR3 | QNDYSYPYT |
| (IMGT) | | |
| SEQ ID NO: 204 | VL | EIVLTQSPATLSLSPGERATLS |
| | | CKSSQSLLDSGNQKNFLTWYQQKPG |
| | | QAPRLLIYWASTRESGVPSRFSGSGS |
| | | GTDFTFTISSLEAEDAATYYCQNDYS |
| | | YPYTFGQGTKVEIK |

In embodiments, an inhibitor of PD-1 is a molecule other than an antibody or fragment thereof. In embodiments, an inhibitor of PD-1 comprises a RNA molecule, e.g., dsRNA molecule, e.g., a a dsRNA molecule (e.g., an RNAi agents such as a shRNA, siRNA, miRNA, clustered regularly interspaced short palindromic repeats (CRISPR), transcription-activator like effector nuclease (TALEN), or zinc finger endonuclease (ZFN)) that targets and modulates or regulates, e.g., inhibits, PD-1, as described, e.g., in paragraph [00489] and Tables 16 and 17 of International Publication WO2015/090230, filed Dec. 19, 2014, which is incorporated by reference in its entirety.

Antibodies, antibody fragments, and other inhibitors of PD-1, PD-L1 and PD-L2 are available in the art and may be used combination with a CAR-expressing cell of the present disclosure described herein. In some embodiments, the PD-1 inhibitor is chosen from PDR001 (Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune).

Nivolumab (also referred to as BMS-936558 or MDX1106; Bristol-Myers Squibb) is a fully human IgG4 monoclonal antibody which specifically blocks PD-1. Nivolumab (clone 5C4) and other human monoclonal antibodies that specifically bind to PD-1 are disclosed in U.S. Pat. No. 8,008,449 and WO2006/121168.

In some embodiments, the anti-PD-1 antibody is Nivolumab. Alternative names for Nivolumab include MDX-1106, MDX-1106-04, ONO-4538, OPDIVO® or BMS-936558. In some embodiments, the anti-PD-1 antibody is Nivolumab (CAS Registry Number: 946414-94-4). Nivolumab is a fully human IgG4 monoclonal antibody which specifically blocks PD1. Nivolumab (clone 5C4) and other human monoclonal antibodies that specifically bind to PD1 are disclosed in U.S. Pat. No. 8,008,449 and WO2006/121168. In one embodiment, the inhibitor of PD-1 is Nivolumab, and having a sequence disclosed herein (or a sequence substantially identical or similar thereto, e.g., a sequence at least 85%, 90%, 95% identical or higher to the sequence specified). In one embodiment, the anti-PD-1 antibody molecule comprises one or more of the CDR sequences (or collectively all of the CDR sequences), the heavy chain or light chain variable region sequence, or the heavy chain or light chain sequence of Nivolumab.

The heavy and light chain amino acid sequences of Nivolumab are as follows:

Heavy chain
(SEQ ID NO: 281)
QVQLVESGGGVVQPGRSLRLDCKASGITFSNSGMHWVRQAPGKGLEWVA
VIWYDGSKRYYADSVKGRFTISRDNSKNTLFLQMNSLRAEDTAVYYCAT
NDDYWGQGTLVTVSSASTKGPSVFPLAPCSRSTSESTAALGCLVKDYFP
EPVTVSWNSGALTSGVHTFPAVLQSSGLYSLSSVVTVPSSSLGTKTYTC
NVDHKPSNTKVDKRVESKYGPPCPPCPAPEFLGGPSVFLFPPKPKDTLM
ISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGK
Light chain
(SEQ ID NO: 282)
EIVLTQSPATLSLSPGERATLSCRASQSVSSYLAWYQQKPGQAPRLLIY
DASNRATGIPARFSGSGSGTDFTLTISSLEPEDFAVYYCQQSSNWPRTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

Pembrolizumab (formerly known as lambrolizumab, and also referred to as MK03475; Merck) is a humanized IgG4 monoclonal antibody that binds to PD-1. Pembrolizumab and other humanized anti-PD-1 antibodies are disclosed in U.S. Pat. No. 8,354,509 and WO2009/114335. AMP-224 (B7-DCIg; Amplimmune; e.g., disclosed in WO2010/027827 and WO2011/066342), is a PD-L2 Fc fusion soluble receptor that blocks the interaction between PD-1 and B7-H1. Other anti-PD-1 antibodies include AMP 514 (Amplimmune), among others, e.g., anti-PD-1 antibodies disclosed in U.S. Pat. No. 8,609,089, US 2010028330, and/or US 20120114649.

In some embodiments, the anti-PD-1 antibody is Pembrolizumab. Pembrolizumab (also referred to as Lambrolizumab, MK-3475, MK03475, SCH-900475 or KEYTRUDA®; Merck) is a humanized IgG4 monoclonal antibody that binds to PD-1. Pembrolizumab and other humanized anti-PD-1 antibodies are disclosed in Hamid, O. et al. (2013) New England Journal of Medicine 369 (2): 134-44, U.S. Pat. No. 8,354,509 and WO2009/114335.

Pembrolizumab

In one embodiment, the inhibitor of PD-1 is Pembrolizumab disclosed in, e.g., U.S. Pat. No. 8,354,509 and WO 2009/114335, and having a sequence disclosed herein (or a sequence substantially identical or similar thereto, e.g., a sequence at least 85%, 90%, 95% identical or higher to the sequence specified). In one embodiment, the anti-PD-1 antibody molecule comprises one or more of the CDR sequences (or collectively all of the CDR sequences), the heavy chain or light chain variable region sequence, or the heavy chain or light chain sequence of Pembrolizumab.

In some embodiments, the anti-PD1 antibody molecule comprises:

(i) a heavy chain variable (VH) region comprising a VHCDR1 amino acid sequence of SEQ ID NO: 530; a VHCDR2 amino acid sequence of SEQ ID NO: 531; and a VHCDR3 amino acid sequence of SEQ ID NO: 532; and
(ii) a light chain variable (VL) region comprising a VLCDR1 amino acid sequence of SEQ ID NO: 527; a VLCDR2 amino acid sequence of SEQ ID NO: 528; and a VLCDR3 amino acid sequence of SEQ ID NO: 529,
or a sequence similar thereto, e.g., a sequence at least 85%, 90%, 95% identical or higher.

In other embodiments, the anti-PD1 antibody molecule comprises a heavy chain comprising the amino acid of SEQ ID NO: 283, and a light chain comprising the amino acid of SEQ ID NO: 284, or a sequence identical or similar thereto, e.g., a sequence at least 85%, 90%, 95% identical or higher.

Amino acid sequences of the heavy chain, light chain, heavy chain CDRs, and light chain CDRs of Pembrolizumab are as disclosed below:

| Heavy chain | |
|---|---|
| (SEQ ID NO: 283) | |
| QVQLVQSGVE VKKPGASVKV SCKASGYTFT NYYMYWVRQA PGQGLEWMGG | 50 |
| INPSNGGTNF NEKFKNRVTL TTDSSTTTAY MELKSLQFDD TAVYYCARRD | 100 |
| YRFDMGFDYW GQGTTVTVSS ASTKGPSVFP LAPCSRSTSE STAALGCLVK | 150 |
| DYFPEPVTVS WNSGALTSGV HTFPAVLQSS GLYSLSSVVT VPSSSLGTKT | 200 |
| YTCNVDHKPS NTKVDKRVES KYGPPCPPCP APEFLGGPSV FLFPPKPKDT | 250 |
| LMISRTPEVT CVVVDVSQED PEVQFNWYVD GVEVHNAKTK PREEQFNSTY | 300 |
| RVVSVLTVLH QDWLNGKEYK CKVSNKGLPS SIEKTISKAK GQPREPQVYT | 350 |
| LPPSQEEMTK NQVSLTCLVK GFYPSDIAVE WESNGQPENN YKTTPPVLDS | 400 |
| DGSFFLYSRL TVDKSRWQEG NVFSCSVMHE ALHNHYTQKS LSLSLGK | 447 |
| Light chain | |
| (SEQ ID NO: 284) | |
| EIVLTQSPAT LSLSPGERAT LSCRASKGVS TSGYSYLHWY QQKPGQAPRL | 50 |
| LIYLASYLES GVPARFSGSG SGTDFTLTIS SLEPEDFAVY YCQHSRDLPL | 100 |
| TFGGGTKVEI KRTVAAPSVF IFPPSDEQLK SGTASVVCLL NNFYPREAKV | 150 |
| QWKVDNALQS GNSQESVTEQ DSKDSTYSLS STLTLSKADY EKHKVYACEV | 200 |
| THQGLSSPVT KSFNRGEC | 218 |

Light chain CDR1:
(SEQ ID NO: 527)
RASKGVSTSGYSYLH
Light chain CDR2:
(SEQ ID NO: 528)
LASYLES
Light chain CDR3:
(SEQ ID NO: 529)
QHSRDLPLT
Heavy chain CDR1:
(SEQ ID NO: 530)
NYYMY
Heavy chain CDR2:
(SEQ ID NO: 531)
GINPSNGGTNFNEKFKN
Heavy chain CDR3:
(SEQ ID NO: 532)
RDYRFDMGFDY

In some embodiments, the anti-PD-1 antibody is Pidilizumab. Pidilizumab (CT-011; Cure Tech) is a humanized IgG1k monoclonal antibody that binds to PD1. Pidilizumab and other humanized anti-PD-1 monoclonal antibodies are disclosed in WO2009/101611, Rosenblatt, J. et al. (2011) J Immunotherapy 34(5): 409-18, U.S. Pat. Nos. 7,695,715, 7,332,582, and 8,686,119, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Pidilizumab.

In one embodiment, the anti-PD-1 antibody molecule is MEDI0680 (Medimmune), also known as AMP-514. MEDI0680 and other anti-PD-1 antibodies are disclosed in U.S. Pat. No. 9,205,148 and WO 2012/145493, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of MEDI0680.

In one embodiment, the anti-PD-1 antibody molecule is REGN2810 (Regeneron). In one embodiment, the anti-PD-1 antibody molecule comprises one or more of the CDR sequences (or collectively all of the CDR sequences), the heavy chain or light chain variable region sequence, or the heavy chain or light chain sequence of REGN2810.

In one embodiment, the anti-PD-1 antibody molecule is PF-06801591 (Pfizer). In one embodiment, the anti-PD-1 antibody molecule comprises one or more of the CDR sequences (or collectively all of the CDR sequences), the heavy chain or light chain variable region sequence, or the heavy chain or light chain sequence of PF-06801591.

In one embodiment, the anti-PD-1 antibody molecule is BGB-A317 or BGB-108 (Beigene). In one embodiment, the anti-PD-1 antibody molecule comprises one or more of the CDR sequences (or collectively all of the CDR sequences), the heavy chain or light chain variable region sequence, or the heavy chain or light chain sequence of BGB-A317 or BGB-108.

In one embodiment, the anti-PD-1 antibody molecule is INCSHR1210 (Incyte), also known as INCSHR01210 or SHR-1210. In one embodiment, the anti-PD-1 antibody molecule comprises one or more of the CDR sequences (or collectively all of the CDR sequences), the heavy chain or light chain variable region sequence, or the heavy chain or light chain sequence of INCSHR1210.

In one embodiment, the anti-PD-1 antibody molecule is TSR-042 (Tesaro), also known as ANB011. In one embodiment, the anti-PD-1 antibody molecule comprises one or more of the CDR sequences (or collectively all of the CDR sequences), the heavy chain or light chain variable region sequence, or the heavy chain or light chain sequence of TSR-042.

Other anti-PD1 antibodies include AMP 514 (Amplimmune), among others, e.g., anti-PD1 antibodies disclosed in U.S. Pat. No. 8,609,089, US 2010028330, and/or US 20120114649. Further known anti-PD-1 antibodies include those described, e.g., in WO 2015/112800, WO 2016/092419, WO 2015/085847, WO 2014/179664, WO 2014/194302, WO 2014/209804, WO 2015/200119, U.S. Pat. Nos. 8,735,553, 7,488,802, 8,927,697, 8,993,731, and 9,102,727, incorporated by reference in their entirety.

In one embodiment, the anti-PD-1 antibody is an antibody that competes for binding with, and/or binds to the same epitope on PD-1 as, one of the anti-PD-1 antibodies described herein.

In one embodiment, the PD-1 inhibitor is a peptide that inhibits the PD-1 signaling pathway, e.g., as described in U.S. Pat. No. 8,907,053, incorporated by reference in its entirety. In some embodiments, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In some embodiments, the PD-1 inhibitor is AMP-224 (B7-DCIg; Amplimmune; e.g., disclosed in WO2010/027827 and WO2011/066342), is a PD-L2 Fc fusion soluble receptor that blocks the interaction between PD-1 and B7-H1.

In one embodiment, the anti-PD-1 antibody or fragment thereof is an anti-PD-1 antibody molecule as described in US 2015/0210769, entitled “Antibody Molecules to PD-1 and Uses Thereof,” incorporated by reference in its entirety. In one embodiment, the anti-PD-1 antibody molecule includes at least one, two, three, four, five or six CDRs (or collectively all of the CDRs) from a heavy and light chain variable region from an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 1 of US 2015/0210769, or a sequence substantially identical (e.g., at least 80%, 85%, 90%, 92%, 95%, 97%, 98%, 99% or higher identical) to any of the aforesaid sequences; or closely related CDRs, e.g., CDRs which are identical or which have at least one amino acid alteration, but not more than two, three or four alterations (e.g., substitutions, deletions, or insertions, e.g., conservative substitutions).

In yet another embodiment, the anti-PD-1 antibody molecule comprises at least one, two, three or four variable regions from an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 1 of US 2015/0210769; or a sequence substantially identical (e.g., at least 80%, 85%, 90%, 92%, 95%, 97%, 98%, 99% or higher identical) to any of the aforesaid sequences.

### Therapeutic Application for Diseases and Disorders

Antigen, e.g., EGFRvIII, Associated Diseases and/or Disorders

The present disclosure provides compositions and methods for treating diseases and disorders (e.g., cancers), e.g., associated with the expression of an antigen, e.g., EGFRvIII. In one aspect, the invention provides methods for treating a disease wherein part of the cancer is negative for the antigen, e.g., EGFRvIII, and part of the cancer is positive for the antigen, e.g., EGFRvIII. For example, the methods and compositions of the invention are useful for treating subjects that have relapsed or have a refractory disease (e.g., cancer, e.g., EGFRvIII+cancer).

EGFRvIII is a tumor specific, ligand-independent, constitutively active variant of the epidermal growth factor receptor. The present invention provides compositions and methods for treating diseases and disorders associated with EGFRvIII. An example of a disease or disorder associated with EGFRvIII is glioma

Glioma refers to a cancer of the central nervous system that begins in glial cells (e.g., cells that surround and support nerve cells and includes oligodendrocytes, astrocytes, microglia, and ependymal cells). Gliomas are particularly serious in terms of both incidence and malignancy, and are classified into seven or more types such as glioblastoma and anaplastic astrocytoma according to their detailed pathological tissue type. Disease stage (tumor size, presence of distal metastasis) and histological malignancy are used when determining the degree of malignancy of primary brain tumors. Histological malignancy is classified into four levels, i.e., G1 to G4 according to the Guidelines for the Treatment of Brain Tumors ((2002) Kanehara & Co., Ltd.), and these correspond to WHO1 to WHO4, respectively. The larger the number, the higher the degree of malignancy. For example, the malignancy of glioblastoma is G4 (WHO4), while the malignancy of anaplastic astrocytoma is G3 (WHO3), and both G3 and G4 are classified as malignant. Thus, according to some embodiments, the methods of this invention target malignant gliomas. In other aspects the invention targets glioblastoma multiforme (GBM). In further embodiments, the compositions and methods of the present invention may be used in the treatment of other gliomas including, but not limited to, anaplastic astrocytoma, giant cell glioblastoma, gliosarcoma, anaplastic oligodendroglioma, anaplastic ependymoma, choroid plexus carcinoma, anaplastic ganglioglioma, pineoblastoma, medulloepithelioma, ependymoblastoma, medulloblastoma, supratentorial primitive neuroectodermal tumor, and atypical teratoid/rhabdoid tumor

Glioblastoma is the most common primary brain tumor in adults. More than half of the 18,000 patients diagnosed with malignant primary brain tumors in US each year have glioblastoma multiforme. Glioblastoma multiforme is an anaplastic, highly cellular tumor, with high proliferation indices, microvascular proliferation and focal necrosis. Signs and symptoms depend on several factors (size, rate of growth, localization of the tumor within the brain) and are mainly represented by headache, seizures, neurological deficits, changes in mental status. Glioblastoma multiforme prognosis remains dismal. Survival time is less than 2 years for the majority of patients. Karnofsky performance status (KPS) is one of the most important prognostic factors: patients with KPS>70 are alive at 18 months in approx 18% of cases, compared with 13% of patients with lower KPS scores. Primary glioblastoma multiforme develops de novo from glial cells, typically has a clinical history of less than six months, is more common in older patients and presents small-cell histology. Secondary glioblastoma multiforme develops over months or years from pre-existing low-grade astrocytomas, predominantly affects younger people and presents giant-cell histology.

Malignant gliomas are also known as high grade gliomas. They can affect the brain and the spinal cord. In some aspects, compositions and methods of the present invention may be used to treat subjects carrying a brain malignant glioma, for example, one that is chosen among anaplastic astrocytoma (AA), glioblastoma multiform (GBM), anaplastic oligodendroglioma (AO) and anaplastic oligoastrocytoma (AOA). In some aspects, compositions and methods of the present invention may be used to treat subjects carrying a glioblastoma multiforme (GBM).

Glioblastoma multiforme is the most malignant stage of astrocytoma, with survival times of less than 2 years for most patients. Histologically, these tumors are characterized by high proliferation indices, endothelial proliferation and focal necrosis. The highly proliferative nature of these lesions likely results from multiple mitogenic effects. One of the hallmarks of GBM is endothelial proliferation. A host of angiogenic growth factors and their receptors are found in GBMs.

There are biologic subsets of astrocytomas, which may reflect the clinical heterogeneity observed in these tumors. These subsets include brain stem gliomas, which are a form of pediatric diffuse, fibrillary astrocytoma that often follow a malignant course. Brain stem GBMs share genetic features with those adult GBMs that affect younger patients. Pleiomorphic xanthoastrocytoma (PXA) is a superficial, low-grade astrocytic tumor that predominantly affects young adults. While these tumors have a bizarre histological appearance, they are typically slow-growing tumors that may be amenable to surgical cure. Some PXAs, however, may recur as GBM. Pilocytic astrocytoma is the most common astrocytic tumor of childhood and differs clinically and histopathologically from the diffuse, fibrillary astrocytoma that affects adults. Pilocytic astrocytomas do not have the same genomic alterations as diffuse, fibrillary astrocytomas. Subependymal giant cell astrocytomas (SEGA) are periventricular, low-grade astrocytic tumors that are usually associated with tuberous sclerosis (TS), and are histologically identical to the so-called “candle-gutterings” that line the ventricles of TS patients. Similar to the other tumorous lesions in TS, these are slowly-growing and may be more akin to hamartomas than true neoplasms. Desmoplastic cerebral astrocytoma of infancy (DCAI) and desmoplastic infantile ganglioglioma (DIGG) are large, superficial, usually cystic, benign astrocytomas that affect children in the first year or two of life.

Oligodendrogliomas and oligoastrocytomas (mixed gliomas) are diffuse, primarily CNS glial tumors that are clinically and biologically most closely related to the diffuse, fibrillary astrocytomas. The tumors, however, are far less common than astrocytomas and have generally better prognoses than the diffuse astrocytomas. Oligodendrogliomas and oligoastrocytomas may progress, either to WHO grade III anaplastic oligodendroglioma or anaplastic oligoastrocytoma, or to WHO grade IV GBM. Thus, the genetic changes that lead to oligodendroglial tumors constitute yet another pathway to GBM.

Ependymomas are a clinically diverse group of gliomas that vary from aggressive intraventricular tumors of children to benign spinal cord tumors in adults. Transitions of ependymoma to GBM are rare. Choroid plexus tumors are also a varied group of tumors that preferentially occur in the ventricular system, ranging from aggressive supratentorial intraventricular tumors of children to benign cerebellopontine angle tumors of adults. Choroid plexus tumors have been reported occasionally in patients with Li-Fraumeni syndrome and von Hippel-Lindau (VHL) disease.

Medulloblastomas are malignant, primitive tumors that arise in the posterior fossa, primarily in children. These tumors also occur in young adults. Medulloblastomas often are surgically resected with subsequent treatment with chemotherapy and/or radiation. They may recur locally or occasionally as drop metastasis from the posterior fossa to the spine. Meningiomas are common intracranial tumors that arise in the meninges and compress the underlying brain. Although typically considered benign and only rarely frankly malignant, management of these tumors often poses clinical challenges. Histological grades of meningiomas vary with the majority benign, WHO grade I/IV (82%); less commonly atypical, WHO II/IV (15%); and infrequently they occur as anaplastic or malignant, WHO grade III/IV (3%).

Schwannomas are benign tumors that arise on peripheral nerves. Schwannomas may arise on cranial nerves, particularly the vestibular portion of the eighth cranial nerve (vestibular schwannomas, acoustic neuromas) where they present as cerebellopontine angle masses. Hemangioblastomas are tumors of uncertain origin that are composed of endothelial cells, pericytes and so-called stromal cells. These benign tumors most frequently occur in the cerebellum and spinal cord of young adults. Multiple hemangioblastomas are characteristic of von Hippel-Lindau disease (VHL). Hemangiopericytomas (HPCs) are dural tumors which may display locally aggressive behavior and may metastasize. The histogenesis of dural-based hemangiopericytoma (HPC) has long been debated, with some authors classifying it as a distinct entity and others classifying it as a subtype of meningioma.

The symptoms of both primary and metastatic brain tumors often depend on the location in the brain and the size of the tumor. Since various regions of the brain are responsible for specific functions, clinical symptoms will vary a great deal. Tumors in the frontal lobe of the brain may cause weakness and paralysis, mood disturbances, difficulty thinking, confusion and disorientation, and wide emotional mood swings. Parietal lobe tumors may cause seizures, numbness or paralysis, difficulty with handwriting, inability to perform simple mathematical problems, difficulty with certain movements, and loss of the sense of touch. Tumors in the occipital lobe can cause loss of vision in half of each visual field, visual hallucinations, and seizures. Temporal lobe tumors can cause seizures, perceptual and spatial disturbances, and receptive aphasia. If a tumor occurs in the cerebellum, the person may have ataxia, loss of coordination, headaches, and vomiting. Tumors in the hypothalamus may cause emotional changes, and changes in the perception of hot and cold. In addition, hypothalamic tumors may affect growth and nutrition in children. With the exception of the cerebellum, a tumor on one side of the brain causes symptoms and impairment on the opposite side of the body.

Compositions and methods of the present invention may be used to treat a subject who has been characterized as having cells or tissues expressing EGFRvIII, or is suspected of having cells or tissues expressing EGFRvIII. For example, subjects benefiting from treatment according to the invention include subjects with a glioma, or subjects suspected of having a glioma, for example, as evidenced by the presence of one or more of headaches, nausea and vomiting, seizures, loss of vision, pain, weakness, numbness in the extremities, and/or cranial nerve disorders as a result of increased intracranial pressure. In particular embodiments, the glioma being treated is glioblastoma multiforme. In accordance with this embodiment, the glioblastoma multiforme can be in the brain or spinal cord.

In certain embodiments, the subject has previously been administered a chemotherapy, e.g., a chemotherapy described herein prior to administration with a CAR-expressing cell and/or a PD-1 inhibitor described herein. In embodiments, the subject has previously been administered an immunotherapy prior to administration with a CAR-expressing cell and/or a PD-1 inhibitor described herein. In embodiments, the subject has previously undergone radiation therapy prior to administration with a CAR-expressing cell and/or a PD-1 inhibitor described herein.

Exemplary cancers that can be treated with the combination therapy described herein (e.g., CAR-expressing cell and a PD-1 inhibitor) include GBM. Exemplary cancers are described in greater detail below.

The disclosure includes (among other things) a type of cellular therapy where T cells are genetically modified to express a chimeric antigen receptor (CAR) and the CAR T cell is infused to a recipient in need thereof. The infused cell is able to kill tumor cells in the recipient. Unlike antibody therapies, CAR-modified T cells are able to replicate in vivo resulting in long-term persistence that can lead to sustained tumor control. In various aspects, the T cells administered to the patient, or their progeny, persist in the patient for at least four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration of the T cell to the patient.

The invention also includes a type of cellular therapy where immune effector cells, e.g., NK cells or T cells are modified, e.g., by in vitro transcribed RNA, to transiently express a chimeric antigen receptor (CAR) and the CAR-expressing (e.g., CART) cell is infused to a recipient in need thereof. The infused cell is able to kill cancer cells in the recipient. Thus, in various aspects, the CAR-expressing cells, e.g., T cells, administered to the patient, is present for less than one month, e.g., three weeks, two weeks, one week, after administration of the CAR-expressing cell, e.g., T cell, to the patient.

Without wishing to be bound by any particular theory, the anti-cancer immunity response elicited by the CAR-modified T cells may be an active or a passive immune response, or alternatively may be due to a direct vs indirect immune response. In one aspect, the CAR (e.g., EGFRvIII-CAR) transduced T cells exhibit specific proinflammatory cytokine secretion and potent cytolytic activity in response to human cancer cells expressing the target antigen (e.g., EGFRvIII), resist soluble target antigen inhibition, mediate bystander killing and mediate regression of an established human cancer. For example, antigen-less cancer cells within a heterogeneous field of target antigen-expressing cancer may be susceptible to indirect destruction by target antigen-redirected T cells that has previously reacted against adjacent antigen-positive cancer cells.

In one aspect, the disclosure features a method of treating cancer in a subject. The method comprises administering to the subject a combination therapy that includes administering a CAR-expressing cell (e.g., EGFRvIII CAR-expressing cell) and a PD-1 inhibitor such that the cancer is treated in the subject. An example of a cancer that is treatable by the combination therapy described herein is a cancer associated with expression of an antigen, e.g., EGFRvIII. In one aspect, the cancer associated with expression of an antigen, e.g., EGFRvIII, is selected from any of the 1 cancers described herein, e.g., GBM, e.g. IDH-wildtype GBM, IDH-mutant GBM, or MGMT-unmethylated GBM.

In one embodiment, the combination therapy of a CAR-expressing cell (e.g., EGFRvIII CAR-expressing cell) and a PD-1 inhibitor described herein results in one or more of: improved or increased anti-tumor activity of the CAR-expressing cell (e.g., EGFRvIII CAR-expressing cell); increased proliferation or persistence of the CAR-expressing cell; improved or increased infiltration of the CAR-expressing cell; improved inhibition of tumor progression; delay of tumor progression; inhibition or reduction in cancer cell proliferation; and/or reduction in tumor burden, e.g., tumor volume, or size, e.g., as compared to a monotherapy of CAR-expressing cell or PD-1 inhibitor alone. In one embodiment, the combination therapy results in increased persistence of the CAR-expressing cell and/or increased persistence of the CAR-expressing cell and a lower, e.g., reduced, risk of relapse.

The present invention provides methods for inhibiting the proliferation of or reducing an antigen-expressing (e.g., EGFRvIII-expressing) cell population. In one embodiment, the methods comprise administering a combination therapy, e.g., a combination comprising a CAR-expressing cell (e.g., EGFRvIII CAR-expressing cell), or a population of CAR expressing cells, and a PD-1 inhibitor. In certain embodiments, the combination therapy described herein reduces the quantity, number, amount or percentage of cells and/or cancer cells by at least at least 5%, 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% in a subject with or animal model of an antigen (e.g., EGFRvIII) or another cancer associated with antigen-expressing (e.g. EGFRvIII-expressing) cells relative to the quantity, number, amount, or percentage of cells and/or cancer cells in a subject treated with a CAR-expressing (e.g., EGFRvIII CAR-expressing) cell or a PD-1 inhibitor alone. In one embodiment, the subject is a human. In an embodiment, the subject is a monkey, e.g., cynomolgus monkey.

The invention also provides methods for preventing, treating and/or managing a disorder, e.g., a disorder associated with antigen-expressing cells (e.g., EGFRvIII-expressing cells) (e.g., a cancer described herein), the methods comprising administering to a subject in need a CAR-expressing cell (e.g., EGFRvIII CAR-expressing cell), or a population of CAR-expressing cells, and a PD-1 inhibitor. In one aspect, the subject is a human.

In one aspect, the invention pertains to a method of inhibiting growth of a cancer cell, (e.g., an antigen-expressing, e.g., EGFRvIII-expressing, cancer cell), comprising contacting the cancer cell with a CAR-expressing (e.g., EGFRvIII CAR expressing) cell, e.g., an EGFRvIII CART cell, described herein, and one or more other CAR expressing cells, e.g., as described herein, such that the CART is activated in response to the antigen and targets the cancer cell, wherein the growth of the cancer is inhibited. The CAR-expressing cell, e.g., T cell, is administered in combination with a PD-1, e.g., a PD-1 described herein.

The present disclosure also provides methods for preventing, treating and/or managing a disease, e.g., a disease associated with antigen-expressing (e.g., EGFRvIII-expressing) cells (e.g., a cancer expressing the antigen, e.g., EGFRvIII), the methods comprising administering to a subject in need an CAR-expressing (e.g., EGFRvIII CAR-expressing) cell that binds to the antigen-expressing cell and administering a PD-1 inhibitor described herein. In one aspect, the subject is a human.

The present disclosure also provides methods for preventing, treating and/or managing a disease associated with antigen-expressing (e.g., EGFRvIII-expressing) cells, the methods comprising administering to a subject in need a CART cell (e.g., an anti-EGFRvIII CART cell) of the invention that binds to the antigen-expressing (e.g., EGFRvIII-expressing) cell. In one aspect, the subject is a human.

The present disclosure also provides methods for preventing relapse of cancer, e.g., associated with antigen-expressing (e.g., EGFRvIII-expressing) cells, the methods comprising administering to a subject in need thereof a CART cell (e.g., an anti-EGFRvIII CART cell) of the invention that binds to the antigen-expressing (e.g., EGFRvIII-expressing) cell. In one aspect, the methods comprise administering to the subject in need thereof an effective amount of a CART cell (e.g., an anti-EGFRvIII CART cell) described herein that binds to the antigen-expressing (e.g., EGFRvIII-expressing) cell in combination with an effective amount of another therapy, e.g., PD-1 inhibitor.

### Combination Therapies

Any of the methods described herein may be used in combination with other known agents and therapies.

The combination described herein, e.g., a CAR-expressing cell (e.g., EGFRvIII CAR-expressing cell) and a PD-1 inhibitor, and the at least one additional therapeutic agent can be administered simultaneously, in the same or in separate compositions, or sequentially. For sequential administration, the CAR-expressing cell and/or the PD-1 inhibitor described herein can be administered after the additional therapeutic agent, or the order of administration can be reversed where the additional therapeutic agent can be administered after the CAR-expressing cell and/or the PD-1 inhibitor described herein. Alternatively, the additional therapeutic agent can be administered between administration of the CAR-expressing cell and the PD-1 inhibitor.

In further aspects, the combination described herein, e.g., a CAR-expressing cell (e.g., EGFRvIII CAR-expressing cell) and a PD-1 inhibitor, may be used in a treatment regimen in combination with surgery, chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludarabine, cyclosporin, FK506, rapamycin, mycophenolic acid, steroids, FR901228, cytokines, and irradiation. peptide vaccine, such as that described in Izumoto et al. 2008 J Neurosurg 108:963-971. Exemplary immunotherapy approaches for malignant glioma are disclosed in Johnson et al. 2010 Curr Neurol Neurosci Rep 10:259-266. In some embodiments, the combination described herein, e.g., a CAR-expressing cell (e.g., EGFRvIII CAR-expressing cell) and a PD-1 inhibitor, may be used in a treatment regimen in combination an agent targets extracellular matrix proteins, such as tenscin, e.g., an anti-tenascin antibody, e.g., a ²¹¹At-labeled anti-tenascin antibody. In some embodiments, the combination described herein, e.g., a CAR-expressing cell (e.g., EGFRvIII CAR-expressing cell) and a PD-1 inhibitor, may be used in a treatment regimen in combination with an immunomodulatory agent, such as interferon alpha, interferon beta, TGF-β2 peptide inhibitor, or poly-ICLC. In some embodiments, the combination described herein, e.g., a CAR-expressing cell (e.g., EGFRvIII CAR-expressing cell) and a PD-1 inhibitor, may be used in a treatment regimen in combination with a WT1 transcription factor peptide vaccine, such as that described in Izumoto et al. 2008 J Neurosurg 108:963-971.

In some aspects, the combination described herein, e.g., a CAR-expressing cell (e.g. EGFRvIII CAR-expressing cell) and a PD-1 inhibitor, may be used in a treatment in combination with an anti-epileptic agent, e.g., acetazolamide, bivaracetam, carbamazepine, clobazam, clonazepam, eslicarbazepine acetate, ethosuximide, gabapentin, lacosamide, lamotrigine, levetiracetam, oxcarbazepine, perampanel, phenobarbital, phenytoin, piracetam, pregabalin, primidone, rudinamide, sodium valproate, stiripentol, tiagabine, topiramate, valporic acid, vigabatrin, zonisamide. See, Weller et al. Lancet 13.9 (2012):e375-e382. In embodiments, the anti-epileptic agent is administered in an amount effective to prevent seizures before the combination described herein, e.g., a CAR-expressing cell (e.g. EGFRvIII CAR-expressing cell) and a PD-1 inhibitor. In embodiments, administration of the anit-epileptic agent is optionally continued throughout and after administration the combination described herein, e.g., a CAR-expressing cell (e.g. EGFRvIII CAR-expressing cell) and a PD-1 inhibitor. In embodiments, the combination described herein, e.g., a CAR-expressing cell (e.g. EGFRvIII CAR-expressing cell) and a PD-1 inhibitor, may be used in a treatment in combination with an anti-epileptic agent and radiation.

In one embodiment, the combination described herein, e.g., a CAR-expressing cell (e.g., EGFRvIII CAR-expressing cell) and a PD-1 inhibitor, can be used in combination with a chemotherapeutic agent. Exemplary chemotherapeutic agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, tositumomab), an antimetabolite (including, e.g., folic acid antagonists, pyrimidine analogs, purine analogs and adenosine deaminase inhibitors (e.g., fludarabine)), a platinum based agent, an angiogenesis inhibitor (e.g., a VEGF pathway inhibitor, a tyrosine kinase inhibitor (e.g., an EGF pathway inhibitor), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide).

General Chemotherapeutic agents are disclosed on pages 268-269 of International Application WO 2016/164731, filed Apr. 8, 2016, which is incorporated by reference in its entirety. General Chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

Exemplary alkylating agents are disclosed on pages 270-271 of International Application WO 2016/164731, filed Apr. 8, 2016, which is incorporated by reference in its entirety. Further examples include include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Exemplary platinum based agents include, without limitation, carboplatin, cisplatin, and oxaliplatin.

Exemplary angiogenesis inhibitors include, without limitation A6 (Angstrom Pharmacueticals), ABT-510 (Abbott Laboratories), ABT-627 (Atrasentan) (Abbott Laboratories/Xinlay), ABT-869 (Abbott Laboratories), Actimid (CC4047, Pomalidomide) (Celgene Corporation), AdGVPEDF.11D (GenVec), ADH-1 (Exherin) (Adherex Technologies), AEE788 (Novartis), AG-013736 (Axitinib) (Pfizer), AG3340 (Prinomastat) (Agouron Pharmaceuticals), AGX1053 (AngioGenex), AGX51 (AngioGenex), ALN-VSP (ALN-VSP 02) (Alnylam Pharmaceuticals), AMG 386 (Amgen), AMG706 (Amgen), Apatinib (YN968D1) (Jiangsu Hengrui Medicine), AP23573 (Ridaforolimus/MK8669) (Ariad Pharmaceuticals), AQ4N (Novavea), ARQ 197 (ArQule), ASA404 (Novartis/Antisoma), Atiprimod (Callisto Pharmaceuticals), ATN-161 (Attenuon), AV-412 (Aveo Pharmaceuticals), AV-951 (Aveo Pharmaceuticals), Avastin (Bevacizumab) (Genentech), AZD2171 (Cediranib/Recentin) (AstraZeneca), BAY 57-9352 (Telatinib) (Bayer), BEZ235 (Novartis), BIBF1120 (Boehringer Ingelheim Pharmaceuticals), BIBW 2992 (Boehringer Ingelheim Pharmaceuticals), BMS-275291 (Bristol-Myers Squibb), BMS-582664 (Brivanib) (Bristol-Myers Squibb), BMS-690514 (Bristol-Myers Squibb), Calcitriol, CCI-779 (Torisel) (Wyeth), CDP-791 (ImClone Systems), Ceflatonin (Homoharringtonine/HHT) (ChemGenex Therapeutics), Celebrex (Celecoxib) (Pfizer), CEP-7055 (Cephalon/Sanofi), CHIR-265 (Chiron Corporation), NGR-TNF, COL-3 (Metastat) (Collagenex Pharaceuticals), Combretastatin (Oxigene), CP-751,871(Figitumumab) (Pfizer), CP-547,632 (Pfizer), CS-7017 (Daiichi Sankyo Pharma), CT-322 (Angiocept) (Adnexus), Curcumin, Dalteparin (Fragmin) (Pfizer), Disulfiram (Antabuse), E7820 (Eisai Limited), E7080 (Eisai Limited), EMD 121974(Cilengitide) (EMD Pharmaceuticals), ENMD-1198 (EntreMed), ENMD-2076 (EntreMed), Endostar (Simcere), Erbitux (ImClone/Bristol-Myers Squibb), EZN-2208 (Enzon Pharmaceuticals), EZN-2968 (Enzon Pharmaceuticals), GC1008 (Genzyme), Genistein, GSK1363089(Foretinib) (GlaxoSmithKline), GW786034 (Pazopanib) (GlaxoSmithKline), GT-111 (Vascular Biogenics Ltd.), IMC-1121B (Ramucirumab) (ImClone Systems), IMC-18F1 (ImClone Systems), IMC-3G3 (ImClone LLC), INCB007839 (Incyte Corporation), INGN 241 (Introgen Therapeutics), Iressa (ZD1839/Gefitinib), LBH589 (Faridak/Panobinostst) (Novartis), Lucentis (Ranibizumab) (Genentech/Novartis), LY317615 (Enzastaurin) (Eli Lilly and Company), Macugen (Pegaptanib) (Pfizer), MEDI522 (Abegrin) (MedImmune), MLN518(Tandutinib) (Millennium), Neovastat (AE941/Benefin) (Aeterna Zentaris), Nexavar (Bayer/Onyx), NM-3 (Genzyme Corporation), Noscapine (Cougar Biotechnology), NPI-2358 (Nereus Pharmaceuticals), OSI-930 (OSI), Palomid 529 (Paloma Pharmaceuticals, Inc.), Panzem Capsules (2ME2) (EntreMed), Panzem NCD (2ME2) (EntreMed), PF-02341066 (Pfizer), PF-04554878 (Pfizer), PI-88 (Progen Industries/Medigen Biotechnology), PKC412 (Novartis), Polyphenon E (Green Tea Extract) (Polypheno E International, Inc), PPI-2458 (Praecis Pharmaceuticals), PTC299 (PTC Therapeutics), PTK787 (Vatalanib) (Novartis), PXD101 (Belinostat) (CuraGen Corporation), RAD001 (Everolimus) (Novartis), RAF265 (Novartis), Regorafenib (BAY73-4506) (Bayer), Revlimid (Celgene), Retaane (Alcon Research), SN38 (Liposomal) (Neopharm), SNS-032 (BMS-387032) (Sunesis), SOM230(Pasireotide) (Novartis), Squalamine (Genaera), Suramin, Sutent (Pfizer), Tarceva (Genentech), TB-403 (Thrombogenics), Tempostatin (Collard Biopharmaceuticals), Tetrathiomolybdate (Sigma-Aldrich), TG100801 (TargeGen), Thalidomide (Celgene Corporation), Tinzaparin Sodium, TKI258 (Novartis), TRC093 (Tracon Pharmaceuticals Inc.), VEGF Trap (Aflibercept) (Regeneron Pharmaceuticals), VEGF Trap-Eye (Regeneron Pharmaceuticals), Veglin (VasGene Therapeutics), Bortezomib (Millennium), XL184 (Exelixis), XL647 (Exelixis), XL784 (Exelixis), XL820 (Exelixis), XL999 (Exelixis), ZD6474 (AstraZeneca), Vorinostat (Merck), and ZSTK474.

Exemplary Vascular Endothelial Growth Factor (VEGF) receptor inhibitors include, but are not limited to, Bevacizumab (Avastin®), axitinib (Inlyta®); Brivanib alaninate (BMS-582664, (S)-((R)-1-(4-(4-Fluoro-2-methyl-1H-indol-5-yloxy)-5-methylpyrrolo[2,1-f][1,2,4]triazin-6-yloxy)propan-2-yl)2-aminopropanoate); Sorafenib (Nexavar®); Pazopanib (Votrient®); Sunitinib malate (Sutent®); Cediranib (AZD2171, CAS 288383-20-1); Vargatef (BIBF1120, CAS 928326-83-4); Foretinib (GSK1363089); Telatinib (BAY57-9352, CAS 332012-40-5); Apatinib (YN968D1, CAS 811803-05-1); Imatinib (Gleevec®); Ponatinib (AP24534, CAS 943319-70-8); Tivozanib (AV951, CAS 475108-18-0); Regorafenib (BAY73-4506, CAS 755037-03-7); Vatalanib dihydrochloride (PTK787, CAS 212141-51-0); Brivanib (BMS-540215, CAS 649735-46-6); Vandetanib (Caprelsa® or AZD6474); Motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470); Dovitinib dilactic acid (TKI258, CAS 852433-84-2); Linfanib (ABT869, CAS 796967-16-3); Cabozantinib (XL184, CAS 849217-68-1); Lestaurtinib (CAS 111358-88-4); N-[5-[[[5-(1,1-Dimethylethyl)-2-oxazolyl]methyl]thio]-2-thiazolyl]-4-piperidinecarboxamide (BMS38703, CAS 345627-80-7); (3R,4R)-4-Amino-1-((4-((3-methoxyphenyl)amino)pyrrolo[2,1-f][1,2,4]triazin-5-yl)methyl)piperidin-3-ol (BMS690514); N-(3,4-Dichloro-2-fluorophenyl)-6-methoxy-7-[[(3aa,5(3,6aa)-octahydro-2-methylcyclopenta[c]pyrrol-5-yl]methoxy]-4-quinazolinamine (XL647, CAS 781613-23-8); 4-Methyl-3-[[1-methyl-6-(3-pyridinyl)-1H-pyrazolo[3,4-d]pyrimidin-4-yl]amino]-N-[3-(trifluoromethyl)phenyl]-benzamide (BHG712, CAS 940310-85-0); and Aflibercept (Eylea®).

Exemplary EGF pathway inhibitors include, without limitation tyrphostin 46, EKB-569, erlotinib (Tarceva®), gefitinib (Iressa®), erbitux, nimotuzumab, lapatinib (Tykerb®), cetuximab (anti-EGFR mAb), ¹⁸⁸Re-labeled nimotuzumab (anti-EGFR mAb), and those compounds that are generically and specifically disclosed in WO 97/02266, EP 0 564 409, WO 99/03854, EP 0 520 722, EP 0 566 226, EP 0 787 722, EP 0 837 063, U.S. Pat. No. 5,747,498, WO 98/10767, WO 97/30034, WO 97/49688, WO 97/38983 and WO 96/33980. Exemplary EGFR antibodies include, but are not limited to, Cetuximab (Erbitux®); Panitumumab (Vectibix®); Matuzumab (EMD-72000); Trastuzumab (Herceptin®); Nimotuzumab (hR3); Zalutumumab; TheraCIM h-R3; MDX0447 (CAS 339151-96-1); and ch806 (mAb-806, CAS 946414-09-1). Exemplary Epidermal growth factor receptor (EGFR) inhibitors include, but not limited to, Erlotinib hydrochloride (Tarceva®), Gefitnib (Iressa®); N-[4-[(3-Chloro-4-fluorophenyl)amino]-7-[[(3″S″)-tetrahydro-3-furanyl]oxy]-6-quinazolinyl]-4(dimethylamino)-2-butenamide, Tovok®); Vandetanib (Caprelsa®); Lapatinib (Tykerb®); (3R,4R)-4-Amino-1-((4-((3-methoxyphenyl)amino)pyrrolo[2,1-f][1,2,4]triazin-5-yl)methyl)piperidin-3-ol (BMS690514); Canertinib dihydrochloride (CI-1033); 6-[4-[(4-Ethyl-1-piperazinyl)methyl]phenyl]-N-[(1R)-1-phenylethyl]-7H-Pyrrolo[2,3-d]pyrimidin-4-amine (AEE788, CAS 497839-62-0); Mubritinib (TAK165); Pelitinib (EKB569); Afatinib (BIBW2992); Neratinib (HKI-272); N-[4-[[1-[(3-Fluorophenyl)methyl]-1H-indazol-5-yl]amino]-5-methylpyrrolo[2,1-f][1,2,4]triazin-6-yl]-carbamic acid, (3S)-3-morpholinylmethyl ester (BMS599626); N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinamine (XL647, CAS 781613-23-8); and 4-[4-[[(1R)-1-Phenylethyl]amino]-7H-pyrrolo[2,3-d]pyrimidin-6-yl]-phenol (PKI166, CAS 187724-61-4).

Exemplary mTOR inhibitors include, without limitation, rapamycin (Rapamune®), and analogs and derivatives thereof; SDZ-RAD; Temsirolimus (Torisel®; also known as CCI-779); Ridaforolimus (formally known as deferolimus, (1R,2R,4S)-4-[(2R)-2 [(1R,9S,12S,15R,16E,18R,19R,21R,23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.0^{4,9}]hexatriaconta-16,24,26,28-tetraen-12-yl]propyl]-2-methoxycyclohexyl dimethylphosphinate, also known as AP23573 and MK8669, and described in PCT Publication No. WO 03/064383); Everolimus (Afinitor® or RAD001); Rapamycin (AY22989, Sirolimus®); Simapimod (CAS 164301-51-3); (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N²-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine-(SEQ ID NO: 526), inner salt (SF1126, CAS 936487-67-1).

Exemplary immunomodulators include, e.g., afutuzumab (available from Roche®); pegfilgrastim (Neulasta®); lenalidomide (CC-5013, Revlimid®); thalidomide (Thalomid®), actimid (CC4047); and IRX-2 (mixture of human cytokines including interleukin 1, interleukin 2, and interferon γ, CAS 951209-71-5, available from IRX Therapeutics).

Exemplary anthracyclines include, e.g., doxorubicin (Adriamycin® and Rubex®); bleomycin (Lenoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PFS®); mitomycin C (Mutamycin®); geldanamycin; herbimycin; ravidomycin; and desacetylravidomycin.

Exemplary vinca alkaloids include, e.g., vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®).

Exemplary proteosome inhibitors include bortezomib (Velcade®); carfilzomib (PX-171-007, (S)-4-Methyl-N—((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoacetamido)-4-phenylbutanamido)-pentanamide); marizomib (NPI-0052); ixazomib citrate (MLN-9708); delanzomib (CEP-18770); and O-Methyl-N-[(2-methyl-5-thiazolyl)carbonyl]-L-seryl-O-methyl-N-[(1S)-2-[(2R)-2-methyl-2-oxiranyl]-2-oxo-1-(phenylmethyl)ethyl]-L-serinamide (ONX-0912).

Exemplary Phosphoinositide 3-kinase (PI3K) inhibitors include, but are not limited to, 4-[2-(1H-Indazol-4-yl)-6-[[4-(methylsulfonyl)piperazin-1-yl]methyl]thieno[3,2-d]pyrimidin-4-yl]morpholine (also known as GDC 0941 and described in PCT Publication Nos. WO 09/036082 and WO 09/055730); 2-Methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in PCT Publication No. WO 06/122806); 4-(trifluoromethyl)-5-(2,6-dimorpholinopyrimidin-4-yl)pyridin-2-amine (also known as BKM120 or NVP-BKM120, and described in PCT Publication No. WO2007/084786); Tozasertib (VX680 or MK-0457, CAS 639089-54-6); (5Z)-5-[[4-(4-Pyridinyl)-6-quinolinyl]methylene]-2,4-thiazolidinedione (GSK1059615, CAS 958852-01-2); (1E,4S,4aR,5R,6aS,9aR)-5-(Acetyloxy)-1-[(di-2-propenylamino)methylene]-4,4a,5,6,6a,8,9,9a-octahydro-11-hydroxy-4-(methoxymethyl)-4a,6a-dimethyl-cyclopenta[5,6]naphtho[1,2-c]pyran-2,7,10(1H)-trione (PX866, CAS 502632-66-8); and 8-Phenyl-2-(morpholin-4-yl)-chromen-4-one (LY294002, CAS 154447-36-6). Exemplary Protein Kinase B (PKB) or AKT inhibitors include, but are not limited to. 8-[4-(1-Aminocyclobutyl)phenyl]-9-phenyl-1,2,4-triazolo[3,4-f][1,6]naphthyridin-3(2H)-one (MK-2206, CAS 1032349-93-1); Perifosine (KRX0401); 4-Dodecyl-N-1,3,4-thiadiazol-2-yl-benzenesulfonamide (PHT-427, CAS 1191951-57-1); 4-[2-(4-Amino-1,2,5-oxadiazol-3-yl)-1-ethyl-7-[(3S)-3-piperidinylmethoxy]-1H-imidazo[4,5-c]pyridin-4-yl]-2-methyl-3-butyn-2-ol (GSK690693, CAS 937174-76-0); 8-(1-Hydroxyethyl)-2-methoxy-3-[(4-methoxyphenyl)methoxy]-6H-dibenzo[b,d]pyran-6-one (palomid 529, P529, or SG-00529); Tricirbine (6-Amino-4-methyl-8-(β-D-ribofuranosyl)-4H,8H-pyrrolo[4,3,2-de]pyrimido[4,5-c]pyridazine); (αS)-α[[[5-(β-Methyl-1H-indazol-5-yl)-3-pyridinyl]oxy]methyl]-benzeneethanamine (A674563, CAS 552325-73-2); 4-[(4-Chlorophenyl)methyl]-1-(7H-pyrrolo[2,3-d]pyrimidin-4-yl)-4-piperidinamine (CCT128930, CAS 885499-61-6); 4-(4-Chlorophenyl)-4-[441H pyrazol-4-yl)phenyl]-piperidine (AT7867, CAS 857531-00-1); and Archexin (RX-0201, CAS 663232-27-7).

Drugs that inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun. 73:316-321, 1991; Bierer et al., Curr. Opin. Immun. 5:763-773, 1993) can also be used. In a further aspect, the cell compositions of the present invention may be administered to a patient in conjunction with (e.g., before, simultaneously or following) bone marrow transplantation, T cell ablative therapy using chemotherapy agents such as, fludarabine, external-beam radiation therapy (XRT), cyclophosphamide, and/or antibodies such as OKT3 or CAMPATH. In one aspect, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

In one embodiment, the subject can be administered an agent which reduces or ameliorates a side effect associated with the administration of the combination described herein, e.g., a CAR-expressing cell (e.g., EGFRvIII CAR-expressing cell) and a PD-1 inhibitor. Side effects associated with the administration of a CAR-expressing cell include, but are not limited to CRS, and hemophagocytic lymphohistiocytosis (HLH), also termed Macrophage Activation Syndrome (MAS). Symptoms of CRS include high fevers, nausea, transient hypotension, hypoxia, and the like. Accordingly, the methods described herein can comprise administering a the combination described herein, e.g., a CAR-expressing cell (e.g., EGFRvIII CAR-expressing cell) and a PD-1 inhibitor, to a subject and further administering an agent to manage elevated levels of a soluble factor resulting from treatment with a CAR-expressing cell. In one embodiment, the soluble factor elevated in the subject is one or more of IFN-γ, TNFα, IL-2 and IL-6. Therefore, an agent administered to treat this side effect can be an agent that neutralizes one or more of these soluble factors. Such agents include, but are not limited to a steroid, an inhibitor of TNFα, and an inhibitor of IL-6. An example of a TNFα inhibitor is entanercept. An example of an IL-6 inhibitor is Tocilizumab (toc).

In one embodiment, the subject can be administered an agent which enhances the activity of the combination described herein, e.g., a CAR-expressing cell (e.g., EGFRvIII CAR-expressing cell) and a PD-1 inhibitor. For example, in one embodiment, the agent can be an agent which inhibits an inhibitory molecule. Additional inhibitory molecules, e.g., can, in some embodiments, decrease the ability of a CAR-expressing cell to mount an immune effector response. Examples of inhibitory molecules include PD1, PD-L1, CTLA4, TIM3, LAGS, VISTA, BTLA, TIGIT, LAIR1, CD160, 2B4 and TGFR beta. Inhibition of an inhibitory molecule, e.g., by inhibition at the DNA, RNA or protein level, can optimize a CAR-expressing cell performance. In embodiments, an inhibitory nucleic acid, e.g., an inhibitory nucleic acid, e.g., a dsRNA, e.g., an siRNA or shRNA, can be used to inhibit expression of an inhibitory molecule in the CAR-expressing cell. In an embodiment the inhibitor is an shRNA. In an embodiment, the inhibitory molecule is inhibited within a CAR-expressing cell. In these embodiments, a dsRNA molecule that inhibits expression of the inhibitory molecule is linked to the nucleic acid that encodes a component, e.g., all of the components, of the CAR. In one embodiment, the inhibitor of an inhibitory signal can be, e.g., an antibody or antibody fragment that binds to an inhibitory molecule. For example, the agent can be an antibody or antibody fragment that binds to PD1, PD-L1, PD-L2 or CTLA4 (e.g., ipilimumab (also referred to as MDX-010 and MDX-101, and marketed as Yervoy®; Bristol-Myers Squibb; Tremelimumab (IgG2 monoclonal antibody available from Pfizer, formerly known as ticilimumab, CP-675,206).). The agent which enhances the activity of e.g., a CAR-expressing cell (e.g., EGFRvIII CAR-expressing cell) and a PD-1 inhibitor can be, e.g., a fusion protein comprising a first domain and a second domain, wherein the first domain is an inhibitory molecule, or fragment thereof, and the second domain is a polypeptide that is associated with a positive signal, e.g., the polypeptide that is associated with a positive signal is CD28, ICOS, and fragments thereof, e.g., an intracellular signaling domain of CD28 and/or ICOS. In one embodiment, the fusion protein is expressed by the same cell that expressed the CAR. In another embodiment, the fusion protein is expressed by a cell, e.g., a T cell that does not express an anti-EGFRvIII CAR.

In another embodiment, the subjects receive an infusion of the CAR-expressing cell, e.g., compositions of the present disclosure prior to transplantation, e.g., allogeneic stem cell transplant, of cells. In a preferred embodiment, CAR expressing cells transiently express CAR, e.g., by electroporation of an mRNA encoding a CAR, whereby the expression of the CAR is terminated prior to infusion of donor stem cells to avoid engraftment failure.

Some patients may experience allergic reactions to the compounds of the present disclosure and/or other anti-cancer agent(s) during or after administration; therefore, anti-allergic agents are often administered to minimize the risk of an allergic reaction. Suitable anti-allergic agents include corticosteroids, such as dexamethasone (e.g., Decadron®), beclomethasone (e.g., Beclovent®), hydrocortisone (also known as cortisone, hydrocortisone sodium succinate, hydrocortisone sodium phosphate, and sold under the tradenames Ala-Cort®, hydrocortisone phosphate, Solu-Cortef®, Hydrocort Acetate® and Lanacort®), prednisolone (sold under the tradenames Delta-Cortel®, Orapred®, Pediapred® and Prelone®), prednisone (sold under the tradenames Deltasone®, Liquid Red®, Meticorten® and Orasone®), methylprednisolone (also known as 6-methylprednisolone, methylprednisolone acetate, methylprednisolone sodium succinate, sold under the tradenames Duralone®, Medralone®, Medrol®, M-Prednisol® and Solu-Medrol®); antihistamines, such as diphenhydramine (e.g., Benadryl®), hydroxyzine, and cyproheptadine; and bronchodilators, such as the beta-adrenergic receptor agonists, albuterol (e.g., Proventil®), and terbutaline (Brethine®).

Some patients may experience nausea during and after administration of the compound of the present disclosure and/or other anti-cancer agent(s); therefore, anti-emetics are used in preventing nausea (upper stomach) and vomiting. Suitable anti-emetics include aprepitant (Emend®), ondansetron (Zofran®), granisetron HCl (Kytril®), lorazepam (Ativan®. dexamethasone (Decadron®), prochlorperazine (Compazine®), casopitant (Rezonic® and Zunrisa®), and combinations thereof.

Medication to alleviate the pain experienced during the treatment period is often prescribed to make the patient more comfortable. Common over-the-counter analgesics, such Tylenol®, are often used. However, opioid analgesic drugs such as hydrocodone/paracetamol or hydrocodone/acetaminophen (e.g., Vicodin®), morphine (e.g., Astramorph® or Avinza®), oxycodone (e.g., OxyContin® or Percocet®), oxymorphone hydrochloride (Opana®), and fentanyl (e.g., Duragesic®) are also useful for moderate or severe pain.

In an effort to protect normal cells from treatment toxicity and to limit organ toxicities, cytoprotective agents (such as neuroprotectants, free-radical scavengers, cardioprotectors, anthracycline extravasation neutralizers, nutrients and the like) may be used as an adjunct therapy. Suitable cytoprotective agents include Amifostine (Ethyol®), glutamine, dimesna (Tavocept®), mesna (Mesnex®), dexrazoxane (Zinecard® or Totect®), xaliproden (Xaprila®), and leucovorin (also known as calcium leucovorin, citrovorum factor and folinic acid). The structure of the active compounds identified by code numbers, generic or trade names may be taken from the actual edition of the standard compendium “The Merck Index” or from databases, e.g. Patents International (e.g. IMS World Publications).

The above-mentioned compounds, which can be used in combination with a compound of the present disclosure, can be prepared and administered as described in the art, such as in the documents cited above.

In one embodiment, the present disclosure provides pharmaceutical compositions comprising at least one compound of the present disclosure (e.g., a compound of the present disclosure) or a pharmaceutically acceptable salt thereof together with a pharmaceutically acceptable carrier suitable for administration to a human or animal subject, either alone or together with other anti-cancer agents.

In one embodiment, the present disclosure provides methods of treating human or animal subjects suffering from a cellular proliferative disease, such as cancer. The present disclosure provides methods of treating a human or animal subject in need of such treatment, comprising administering to the subject a therapeutically effective amount of a compound of the present disclosure (e.g., a compound of the present disclosure) or a pharmaceutically acceptable salt thereof, either alone or in combination with other anti-cancer agents.

In particular, compositions will either be formulated together as a combination therapeutic or administered separately.

In combination therapy, the compound of the present disclosure and other anti-cancer agent(s) may be administered either simultaneously, concurrently or sequentially with no specific time limits, wherein such administration provides therapeutically effective levels of the two compounds in the body of the patient.

In a preferred embodiment, the compound of the present disclosure and the other anti-cancer agent(s) is generally administered sequentially in any order by infusion or orally. The dosing regimen may vary depending upon the stage of the disease, physical fitness of the patient, safety profiles of the individual drugs, and tolerance of the individual drugs, as well as other criteria well-known to the attending physician and medical practitioner(s) administering the combination. The compound of the present disclosure and other anti-cancer agent(s) may be administered within minutes of each other, hours, days, or even weeks apart depending upon the particular cycle being used for treatment. In addition, the cycle could include administration of one drug more often than the other during the treatment cycle and at different doses per administration of the drug.

In another aspect of the present disclosure, kits that include one or more compound of the present disclosure and a combination partner as disclosed herein are provided. Representative kits include (a) a compound of the present disclosure or a pharmaceutically acceptable salt thereof, (b) at least one combination partner, e.g., as indicated above, whereby such kit may comprise a package insert or other labeling including directions for administration.

A compound of the present disclosure may also be used to advantage in combination with known therapeutic processes, for example, the administration of hormones or especially radiation. A compound of the present disclosure may in particular be used as a radiosensitizer, especially for the treatment of tumors which exhibit poor sensitivity to radiotherapy. In one embodiment, the subject can be administered an agent which reduces or ameliorates a side effect associated with the administration of a CAR-expressing cell. Side effects associated with the administration of a CAR-expressing cell include, but are not limited to CRS, and hemophagocytic lymphohistiocytosis (HLH), also termed Macrophage Activation Syndrome (MAS).

Accordingly, the methods described herein can comprise administering a CAR-expressing cell described herein to a subject and further administering one or more agents to manage elevated levels of a soluble factor resulting from treatment with a CAR-expressing cell. In one embodiment, the soluble factor elevated in the subject is one or more of IFN-γ, TNFα, IL-2 and IL-6. In an embodiment, the factor elevated in the subject is one or more of IL-1, GM-CSF, IL-10, IL-8, IL-5 and fraktalkine. Therefore, an agent administered to treat this side effect can be an agent that neutralizes one or more of these soluble factors. In one embodiment, the agent that neutralizes one or more of these soluble forms is an antibody or antigen binding fragment thereof. Examples of such agents include, but are not limited to a steroid (e.g., corticosteroid), an inhibitor of TNFα, and an inhibitor of IL-6. An example of a TNFα inhibitor is an anti-TNFα antibody molecule such as, infliximab, adalimumab, certolizumab pegol, and golimumab. Another example of a TNFα inhibitor is a fusion protein such as entanercept. Small molecule inhibitor of TNFα include, but are not limited to, xanthine derivatives (e.g. pentoxifylline) and bupropion. An example of an IL-6 inhibitor is an anti-IL-6 antibody molecule such as tocilizumab (toc), sarilumab, elsilimomab, CNTO 328, ALD518/BMS-945429, CNTO 136, CPSI-2364, CDP6038, VX30, ARGX-109, FE301, and FM101. In one embodiment, the anti-IL-6 antibody molecule is tocilizumab. An example of an IL-1R based inhibitor is anakinra.

In some embodiment, the subject is administered a corticosteroid, such as, e.g., methylprednisolone, hydrocortisone, among others.

In some embodiments, the subject is administered a vasopressor, such as, e.g., norepinephrine, dopamine, phenylephrine, epinephrine, vasopressin, or a combination thereof.

In an embodiment, the subject can be administered an antipyretic agent. In an embodiment, the subject can be administered an analgesic agent.

In one embodiment, the subject can be further administered an agent which enhances the activity or fitness of a CAR-expressing cell. For example, in one embodiment, the agent can be an agent which inhibits a molecule that modulates or regulates, e.g., inhibits, T cell function. In some embodiments, the molecule that modulates or regulates T cell function is an inhibitory molecule. Inhibitory molecules, e.g., Programmed Death 1 (PD-1) or PD-1 ligand (PD-L1), can, in some embodiments, decrease the ability of a CAR-expressing cell to mount an immune effector response. Examples of inhibitory molecules include PD-1, PD-L1, CTLA4, TIM3, CEACAM (e.g., CEACAM-1, CEACAM-3 and/or CEACAM-5), LAGS, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGF beta. Inhibition of a molecule that modulates or regulates, e.g., inhibits, T cell function, e.g., by inhibition at the DNA, RNA or protein level, can optimize a CAR-expressing cell performance. In embodiments, an agent, e.g., an inhibitory nucleic acid, e.g., an inhibitory nucleic acid, e.g., an inhibitory nucleic acid, e.g., a dsRNA, e.g., an siRNA or shRNA, a clustered regularly interspaced short palindromic repeats (CRISPR), a transcription-activator like effector nuclease (TALEN), or a zinc finger endonuclease (ZFN), e.g., as described herein, can be used to inhibit expression of an inhibitory molecule in the CAR-expressing cell. In an embodiment, the inhibitor is an shRNA.

In an embodiment, the agent that modulates or regulates, e.g., inhibits, T-cell function is inhibited within a CAR-expressing cell. In these embodiments, a dsRNA molecule that inhibits expression of a molecule that modulates or regulates, e.g., inhibits, T-cell function is linked to the nucleic acid that encodes a component, e.g., all of the components, of the CAR. In an embodiment, a nucleic acid molecule that encodes a dsRNA molecule that inhibits expression of the molecule that modulates or regulates, e.g., inhibits, T-cell function is operably linked to a promoter, e.g., a H1- or a U6-derived promoter such that the dsRNA molecule that inhibits expression of the molecule that modulates or regulates, e.g., inhibits, T-cell function is expressed, e.g., is expressed within a CAR-expressing cell. See e.g., Tiscornia G., “Development of Lentiviral Vectors Expressing siRNA,” Chapter 3, in Gene Transfer: Delivery and Expression of DNA and RNA (eds. Friedmann and Rossi). Cold Spring Harbor Laboratory Press, Cold Spring Harbor, N.Y., USA, 2007; Brummelkamp T R, et al. (2002) Science 296: 550-553; Miyagishi M, et al. (2002) Nat. Biotechnol. 19: 497-500. In an embodiment the nucleic acid molecule that encodes a dsRNA molecule that inhibits expression of the molecule that modulates or regulates, e.g., inhibits, T-cell function is present on the same vector, e.g., a lentiviral vector, that comprises a nucleic acid molecule that encodes a component, e.g., all of the components, of the CAR. In such an embodiment, the nucleic acid molecule that encodes a dsRNA molecule that inhibits expression of the molecule that modulates or regulates, e.g., inhibits, T-cell function is located on the vector, e.g., the lentiviral vector, 5′- or 3′- to the nucleic acid that encodes a component, e.g., all of the components, of the CAR. The nucleic acid molecule that encodes a dsRNA molecule that inhibits expression of the molecule that modulates or regulates, e.g., inhibits, T-cell function can be transcribed in the same or different direction as the nucleic acid that encodes a component, e.g., all of the components, of the CAR. In an embodiment the nucleic acid molecule that encodes a dsRNA molecule that inhibits expression of the molecule that modulates or regulates, e.g., inhibits, T-cell function is present on a vector other than the vector that comprises a nucleic acid molecule that encodes a component, e.g., all of the components, of the CAR. In an embodiment, the nucleic acid molecule that encodes a dsRNA molecule that inhibits expression of the molecule that modulates or regulates, e.g., inhibits, T-cell function it transiently expressed within a CAR-expressing cell. In an embodiment, the nucleic acid molecule that encodes a dsRNA molecule that inhibits expression of the molecule that modulates or regulates, e.g., inhibits, T-cell function is stably integrated into the genome of a CAR-expressing cell. Configurations of exemplary vectors for expressing a component, e.g., all of the components, of the CAR with a dsRNA molecule that inhibits expression of the molecule that modulates or regulates, e.g., inhibits, T-cell function, is provided, e.g., in FIG. 47 of International Publication WO2015/090230, filed Dec. 19, 2014, which is herein incorporated by reference.

In embodiments, the combination described herein, e.g., a CAR-expressing cell (e.g., EGFRvIII CAR-expressing cell) and a PD-1 inhibitor, is administered in combination with an anti-epileptic agent, e.g., acetazolamide, bivaracetam, carbamazepine, clobazam, clonazepam, eslicarbazepine acetate, ethosuximide, gabapentin, lacosamide, lamotrigine, levetiracetam, oxcarbazepine, perampanel, phenobarbital, phenytoin, piracetam, pregabalin, primidone, rudinamide, sodium valproate, stiripentol, tiagabine, topiramate, valporic acid, vigabatrin, zonisamide. See, Weller et al. Lancet 13.9 (2012):e375-e382. In embodiments, the anti-epileptic agent is administered in an amount effective to prevent seizures before the combination described herein, e.g., a CAR-expressing cell (e.g. EGFRvIII CAR-expressing cell) and a PD-1 inhibitor. In embodiments, administration of the anit-epileptic agent is optionally continued throughout and after administration the combination described herein, e.g., a CAR-expressing cell (e.g. EGFRvIII CAR-expressing cell) and a PD-1 inhibitor. In embodiments, the combination described herein, e.g., a CAR-expressing cell (e.g. EGFRvIII CAR-expressing cell) and a PD-1 inhibitor, may be used in a treatment in combination with an anti-epileptic agent and radiation.

### Cytokine Release Syndrome (CRS)

Cytokine release syndrome (CRS) is a potentially life-threatening cytokine-associated toxicity that can occur as a result of cancer immunotherapy, e.g., cancer antibody therapies or T cell immunotherapies (e.g., CAR T cells). CRS results from high-level immune activation when large numbers of lymphocytes and/or myeloid cells release inflammatory cytokines upon activation. The severity of CRS and the timing of onset of symptoms can vary depending on the magnitude of immune cell activation, the type of therapy administered, and/or the extent of tumor burden in a subject. In the case of T-cell therapy for cancer, symptom onset is typically days to weeks after administration of the T-cell therapy, e.g., when there is peak in vivo T-cell expansion. See, e.g., Lee et al. Blood. 124.2(2014): 188-95.

Symptoms of CRS can include neurologic toxicity, disseminated intravascular coagulation, cardiac dysfunction, adult respiratory distress syndrome, renal failure, and/or hepatic failure. For example, symptoms of CRS include high fevers, nausea, transient hypotension, hypoxia, and the like. CRS may include clinical constitutional signs and symptoms such as fever, fatigue, anorexia, myalgias, arthalgias, nausea, vomiting, and headache. CRS may include clinical skin signs and symptoms such as rash. CRS may include clinical gastrointestinal signs and symptoms such as nausea, vomiting and diarrhea. CRS may include clinical respiratory signs and symptoms such as tachypnea and hypoxemia. CRS may include clinical cardiovascular signs and symptoms such as tachycardia, widened pulse pressure, hypotension, increased cardiac output (early) and potentially diminished cardiac output (late). CRS may include clinical coagulation signs and symptoms such as elevated d-dimer, hypofibrinogenemia with or without bleeding. CRS may include clinical renal signs and symptoms such as azotemia. CRS may include clinical hepatic signs and symptoms such as transaminitis and hyperbilirubinemia. CRS may include clinical neurologic signs and symptoms such as headache, mental status changes, confusion, delirium, word finding difficulty or frank aphasia, hallucinations, tremor, dymetria, altered gait, and seizures.

IL-6 is thought to be a mediator of CRS toxicity. See, e.g., id. High IL-6 levels may initiate a proinflammatory IL-6 signaling cascade, leading to one or more of the CRS symptoms. In some cases, the level of C-reactive protein (CRP) (a biomolecule produced by the liver, e.g., in response to IL-6) can be a measure of IL-6 activity. In some cases, CRP levels may increase several fold (e.g., several logs) during CRS. CRP levels can be measured using methods described herein, and/or standard methods available in the art.

CRS Grading

In some embodiments, CRS can be graded in severity from 1-5 as follows. Grades 1-3 are less than severe CRS. Grades 4-5 are severe CRS. For Grade 1 CRS, only symptomatic treatment is needed (e.g., nausea, fever, fatigue, myalgias, malaise, headache) and symptoms are not life threatening. For Grade 2 CRS, the symptoms require moderate intervention and generally respond to moderate intervention. Subjects having Grade 2 CRS develop hypotension that is responsive to either fluids or one low-dose vasopressor; or they develop grade 2 organ toxicity or mild respiratory symptoms that are responsive to low flow oxygen (<40% oxygen). In Grade 3 CRS subjects, hypotension generally cannot be reversed by fluid therapy or one low-dose vasopressor. These subjects generally require more than low flow oxygen and have grade 3 organ toxicity (e.g., renal or cardiac dysfunction or coagulopathy) and/or grade 4 transaminitis. Grade 3 CRS subjects require more aggressive intervention, e.g., oxygen of 40% or higher, high dose vasopressor(s), and/or multiple vasopressors. Grade 4 CRS subjects suffer from immediately life-threatening symptoms, including grade 4 organ toxicity or a need for mechanical ventilation. Grade 4 CRS subjects generally do not have transaminitis. In Grade 5 CRS subjects, the toxicity causes death. For example, criteria for grading CRS is provided herein as Table A. Unless otherwise specified, CRS as used herein refers to CRS according to the criteria of Table A.

TABLE A
CRS grading

| | |
|---|---|
| Gr1 | Supportive care only |
| Gr2 | IV therapies +/− hospitalization. |
| Gr3 | Hypotension requiring IV fluids or low-dose |
| | vasoactives or hypoxemia requiring oxygen, |
| | CPAP, or BIPAP. |
| Gr4 | Hypotension requiring high-dose vasoactives |
| | or hypoxemia requiring mechanical ventilation. |
| Gr 5 | Death |

CRS Therapies

Therapies for CRS include IL-6 inhibitor or IL-6 receptor (IL-6R) inhibitors (e.g., tocilizumab or siltuximab), sgp130 blockers, vasoactive medications, corticosteroids, immunosuppressive agents, and mechanical ventilation. Exemplary therapies for CRS are described in International Application WO2014011984, which is hereby incorporated by reference.

Tocilizumab is a humanized, immunoglobulin G1kappa anti-human IL-6R monoclonal antibody. See, e.g., id. Tocilizumab blocks binding of IL-6 to soluble and membrane bound IL-6 receptors (IL-6Rs) and thus inhibitors classical and trans-IL-6 signaling. In embodiments, tocilizumab is administered at a dose of about 4-12 mg/kg, e.g., about 4-8 mg/kg for adults and about 8-12 mg/kg for pediatric subjects, e.g., administered over the course of 1 hour.

In some embodiments, the CRS therapeutic is an inhibitor of IL-6 signalling, e.g., an inhibitor of IL-6 or IL-6 receptor. In one embodiment, the inhibitor is an anti-IL-6 antibody, e.g., an anti-IL-6 chimeric monoclonal antibody such as siltuximab. In other embodiments, the inhibitor comprises a soluble gp130 or a fragment thereof that is capable of blocking IL-6 signalling. In some embodiments, the sgp130 or fragment thereof is fused to a heterologous domain, e.g., an Fc domain, e.g., is a gp130-Fc fusion protein such as FE301. In embodiments, the inhibitor of IL-6 signalling comprises an antibody, e.g., an antibody to the IL-6 receptor, such as sarilumab, olokizumab (CDP6038), elsilimomab, sirukumab (CNTO 136), ALD518/BMS-945429, ARGX-109, or FM101. In some embodiments, the inhibitor of IL-6 signalling comprises a small molecule such as CPSI-2364.

Exemplary vasoactive medications include but are not limited to angiotensin-11, endothelin-1, alpha adrenergic agonists, rostanoids, phosphodiesterase inhibitors, endothelin antagonists, inotropes (e.g., adrenaline, dobutamine, isoprenaline, ephedrine), vasopressors (e.g., noradrenaline, vasopressin, metaraminol, vasopressin, methylene blue), inodilators (e.g., milrinone, levosimendan), and dopamine.

Exemplary vasopressors include but are not limited to norepinephrine, dopamine, phenylephrine, epinephrine, and vasopressin. In some embodiments, a high-dose vasopressor includes one or more of the following: norpepinephrine monotherapy at ≥20 ug/min, dopamine monotherapy at ≥10 ug/kg/min, phenylephrine monotherapy at ≥200 ug/min, and/or epinephrine monotherapy at ≥10 ug/min. In some embodiments, if the subject is on vasopressin, a high-dose vasopressor includes vasopressin+norepinephrine equivalent of ≥10 ug/min, where the norepinephrine equivalent dose=[norepinephrine (ug/min)]+[dopamine (ug/kg/min)/2]+[epinephrine (ug/min)]+[phenylephrine (ug/min)/10]. In some embodiments, if the subject is on combination vasopressors (not vasopressin), a high-dose vasopressor includes norepinephrine equivalent of ≥20 ug/min, where the norepinephrine equivalent dose=[norepinephrine (ug/min)]+[dopamine (ug/kg/min)/2]+[epinephrine (ug/min)]+[phenylephrine (ug/min)/10]. See e.g., Id.

In some embodiments, a low-dose vasopressor is a vasopressor administered at a dose less than one or more of the doses listed above for high-dose vasopressors.

Exemplary corticosteroids include but are not limited to dexamethasone, hydrocortisone, and methylprednisolone. In embodiments, a dose of dexamethasone of 0.5 mg/kg is used. In embodiments, a maximum dose of dexamethasone of 10 mg/dose is used. In embodiments, a dose of methylprednisolone of 2 mg/kg/day is used.

Exemplary immunosuppressive agents include but are not limited to an inhibitor of TNFα or an inhibitor of IL-1. In embodiments, an inhibitor of TNFα comprises an anti-TNFα antibody, e.g., monoclonal antibody, e.g., infliximab. In embodiments, an inhibitor of TNFα comprises a soluble TNFα receptor (e.g., etanercept). In embodiments, an IL-1 or IL-1R inhibitor comprises anakinra.

In some embodiments, the subject at risk of developing severe CRS is administered an anti-IFN-gamma or anti-sIL2Ra therapy, e.g., an antibody molecule directed against IFN-gamma or sIL2Ra.

In embodiments, for a subject who has received a therapeutic antibody molecule such as blinatumomab and who has CRS or is at risk of developing CRS, the therapeutic antibody molecule is administered at a lower dose and/or a lower frequency, or administration of the therapeutic antibody molecule is halted.

In embodiments, a subject who has CRS or is at risk of developing CRS is treated with a fever reducing medication such as acetaminophen.

In embodiments, a subject herein is administered or provided one or more therapies for CRS described herein, e.g., one or more of IL-6 inhibitors or IL-6 receptor (IL-6R) inhibitors (e.g., tocilizumab), vasoactive medications, corticosteroids, immunosuppressive agents, or mechanical ventilation, in any combination, e.g., in combination with a CAR-expressing cell described herein.

In embodiments, a subject at risk of developing CRS (e.g., severe CRS) (e.g., identified as having a high risk status for developing severe CRS) is administered one or more therapies for CRS described herein, e.g., one or more of IL-6 inhibitor or IL-6 receptor (IL-6R) inhibitors (e.g., tocilizumab), vasoactive medications, corticosteroids, immunosuppressive agents, or mechanical ventilation, in any combination, e.g., in combination with a CAR-expressing cell described herein.

In embodiments, a subject herein (e.g., a subject at risk of developing severe CRS or a subject identified as at risk of developing severe CRS) is transferred to an intensive care unit. In some embodiments, a subject herein (e.g., a subject at risk of developing severe CRS or a subject identified as at risk of developing severe CRS) is monitored for one ore more symptoms or conditions associated with CRS, such as fever, elevated heart rate, coagulopathy, MODS (multiple organ dysfunction syndrome), cardiovascular dysfunction, distributive shock, cardiomyopathy, hepatic dysfunction, renal dysfunction, encephalopathy, clinical seizures, respiratory failure, or tachycardia. In some embodiments, the methods herein comprise administering a therapy for one of the symptoms or conditions associated with CRS. For instance, in embodiments, e.g., if the subject develops coagulopathy, the method comprises administering cryoprecipitate. In some embodiments, e.g., if the subject develops cardiovascular dysfunction, the method comprises administering vasoactive infusion support. In some embodiments, e.g., if the subject develops distributive shock, the method comprises administering alpha-agonist therapy. In some embodiments, e.g., if the subject develops cardiomyopathy, the method comprises administering milrinone therapy. In some embodiments, e.g., if the subject develops respiratory failure, the method comprises performing mechanical ventilation (e.g., invasive mechanical ventilation or noninvasive mechanical ventilation). In some embodiments, e.g., if the subject develops shock, the method comprises administering crystalloid and/or colloid fluids.

In embodiments, the CAR-expressing cell is administered prior to, concurrently with, or subsequent to administration of one or more therapies for CRS described herein, e.g., one or more of IL-6 inhibitor or IL-6 receptor (IL-6R) inhibitors (e.g., tocilizumab), vasoactive medications, corticosteroids, immunosuppressive agents, or mechanical ventilation. In embodiments, the CAR-expressing cell is administered within 2 weeks (e.g., within 2 or 1 week, or within 14 days, e.g., within 14, 13, 12, 11, 10, 9, 8, 7, 6, 5, 4, 3, 2, 1 day or less) of administration of one or more therapies for CRS described herein, e.g., one or more of IL-6 inhibitors or IL-6 receptor (IL-6R) inhibitors (e.g., tocilizumab), vasoactive medications, corticosteroids, immunosuppressive agents, or mechanical ventilation. In embodiments, the CAR-expressing cell is administered at least 1 day (e.g., at least 1, 2, 3, 4, 5, 6, 7, 8, 9, 10, 11, 12, 13, 14, 15, 16, 17, 18, 19, 20, 1, week, 2 weeks, 3 weeks, 4 weeks, 1 month, 2 months, 3 months, 3 months, or more) before or after administration of one or more therapies for CRS described herein, e.g., one or more of IL-6 inhibitors or IL-6 receptor (IL-6R) inhibitors (e.g., tocilizumab), vasoactive medications, corticosteroids, immunosuppressive agents, or mechanical ventilation.

In embodiments, a subject herein (e.g., a subject at risk of developing severe CRS or a subject identified as at risk of developing severe CRS) is administered a single dose of an IL-6 inhibitor or IL-6 receptor (IL-6R) inhibitor (e.g., tocilizumab). In embodiments, the subject is administered a plurality of doses (e.g., 2, 3, 4, 5, 6, or more doses) of an IL-6 inhibitor or IL-6 receptor (IL-6R) inhibitor (e.g., tocilizumab).

In embodiments, a subject at low or no risk of developing CRS (e.g., severe CRS) (e.g., identified as having a low risk status for developing severe CRS) is not administered a therapy for CRS described herein, e.g., one or more of IL-6 inhibitor or IL-6 receptor (IL-6R) inhibitors (e.g., tocilizumab), vasoactive medications, corticosteroids, immunosuppressive agents, or mechanical ventilation.

In some embodiments, the subject treated by the methods disclosed herein has a low severity of CRS, e.g., grade 1, grade 2 or grade 3.

### Pharmaceutical Compositions

Pharmaceutical compositions of the present invention may comprise the combination described herein, e.g., a CAR-expressing cell (e.g., EGFRvIII CAR-expressing cell) and a PD-1 inhibitor, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are in one aspect formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

In one embodiment, the pharmaceutical composition is substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In one embodiment, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

### Methods of Treating

When “an immunologically effective amount,” “an effective dose”, “an anti-cancer effective amount,” “a cancer-inhibiting effective amount,” or “therapeutic amount” is indicated, the precise amount of the compositions of the present invention to be administered can be determined by a physician with consideration of individual differences in age, weight, tumor size, extent of infection or metastasis, and condition of the patient (subject).

The dosage of the above treatments to be administered to a subject will vary with the precise nature of the condition being treated and the recipient of the treatment. The scaling of dosages for human administration can be performed according to art-accepted practices. The dose for CAMPATH, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766).

The administration of the compositions described herein may be carried out in any convenient manner, including by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In one embodiment, the compositions described herein, e.g., comprising a CAR-expressing cell and/or PD-1 inhibitor, are administered to a patient by intradermal or subcutaneous injection. In one embodiment, the the compositions described herein, e.g., comprising a CAR-expressing cell and/or PD-1 inhibitor, are administered by i.v. injection. The the compositions described herein, e.g., comprising a CAR-expressing cell and/or PD-1 inhibitor, may be injected directly into a tumor, lymph node, or site of infection.

It can generally be stated that a pharmaceutical composition comprising the immune effector cells described herein may be administered at a dosage of 10⁴ to 10⁹ cells/kg body weight, in some instances 10⁵ to 10⁶ cells/kg body weight, including all integer values within those ranges. The immune effector cell compositions may also be administered multiple times at these dosages. The cells can be administered by using infusion techniques that are commonly known in immunotherapy (see, e.g., Rosenberg et al., New Eng. J. of Med. 319:1676, 1988).

In certain aspects, it may be desired to administer activated immune effector cells to a subject and then subsequently redraw blood (or have an apheresis performed), activate the cells therefrom according to the present invention, and reinfuse the patient with these activated and expanded cells. This process can be carried out multiple times every few weeks. In certain aspects, the cells can be activated from blood draws of from 10 cc to 400 cc. In certain aspects, the cells are activated from blood draws of 20 cc, 30 cc, 40 cc, 50 cc, 60 cc, 70 cc, 80 cc, 90 cc, or 100 cc.

In a particular exemplary aspect, subjects may undergo leukapheresis, wherein leukocytes are collected, enriched, or depleted ex vivo to select and/or isolate the cells of interest, e.g., T cells. These T cell isolates may be expanded by methods known in the art and treated such that one or more CAR constructs of the invention may be introduced, thereby creating a CAR T cell of the invention. Subjects in need thereof may subsequently undergo standard treatment with high dose chemotherapy followed by peripheral blood stem cell transplantation. In certain aspects, following or concurrent with the transplant, subjects receive an infusion of the expanded CAR expressing cells of the present invention. In an additional aspect, expanded cells are administered before or following surgery.

In one embodiment, the CAR is introduced into immune effector cells, e.g., using in vitro transcription, and the subject (e.g., human) receives an initial administration of CAR-expressing cells of the invention, and one or more subsequent administrations of the CAR-expressing cells of the invention, wherein the one or more subsequent administrations are administered less than 15 days, e.g., 14, 13, 12, 11, 10, 9, 8, 7, 6, 5, 4, 3, or 2 days after the previous administration. In one embodiment, more than one administration of the CAR-expressing cells of the invention are administered to the subject (e.g., human) per week, e.g., 2, 3, or 4 administrations of the CAR-expressing cells of the invention are administered per week. In one embodiment, the subject (e.g., human subject) receives more than one administration of the CAR-expressing cells per week (e.g., 2, 3 or 4 administrations per week) (also referred to herein as a cycle), followed by a week of no CAR-expressing cells administration, and then one or more additional administration of the CAR-expressing cells (e.g., more than one administration of the CAR-expressing cells per week) is administered to the subject. In another embodiment, the subject (e.g., human subject) receives more than one cycle of CAR-expressing cells, and the time between each cycle is less than 10, 9, 8, 7, 6, 5, 4, or 3 days. In one embodiment, the CAR-expressing cells are administered every other day for 3 administrations per week. In one embodiment, the CAR-expressing cells of the invention are administered for at least two, three, four, five, six, seven, eight or more weeks.

In some embodiments, a dose of CAR-expressing cells (e.g., CAR-expressing cells described herein, e.g., EGFRvIII CAR-expressing cells described herein) comprises about 10⁴ to about 10⁹ cells/kg, e.g., about 10⁴ to about 10⁵ cells/kg, about 10⁵ to about 10⁶ cells/kg, about 10⁶ to about 10⁷ cells/kg, about 10⁷ to about 10⁸ cells/kg, or about 10⁸ to about 10⁹ cells/kg. In embodiments, the dose of CAR-expressing cells comprises about 0.6×10⁶ cells/kg to about 2×10⁷ cells/kg. In some embodiments, a dose of CAR-expressing cells described herein (e.g., EGFRvIII CAR-expressing cell) comprises about 2×10⁵, 1×10⁶, 1.1×10⁶, 2×10⁶, 3×10⁶, 3.6×10⁶, 5×10⁶, 1×10⁷, 1.8×10⁷, 2×10⁷, 5×10⁷, 1×10⁸, 2×10⁸, 3×10⁸, or 5×10⁸ cells/kg. In some embodiments, a dose of CAR cells (e.g., EGFRvIII CAR-expressing cell) comprises at least about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, 3×10⁸, or 5×10⁸ cells/kg. In some embodiments, a dose of CAR cells (e.g., EGFRvIII CAR-expressing cell) comprises up to about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, 3×10⁸, or 5×10⁸ cells/kg. In some embodiments, a dose of CAR cells (e.g., EGFRvIII CAR-expressing cell) comprises about 1.1×10⁶-1.8×10⁷ cells/kg. In some embodiments, a dose of CAR cells (e.g., EGFRvIII CAR-expressing cell) comprises about 1×10⁷, 2×10⁷, 5×10⁷, 1×10⁸, 2×10⁸, 3×10⁸, 5×10⁸, 1×10⁹, 2×10⁹, or 5×10⁹ cells. In some embodiments, a dose of CAR cells (e.g., e.g., EGFRvIII CAR-expressing cell) comprises at least about 1×10⁷, 2×10⁷, 5×10⁷, 1×10⁸, 2×10⁸, 3×10⁸, 5×10⁸, 1×10⁹, 2×10⁹, or 5×10⁹ cells. In some embodiments, a dose of CAR cells (e.g., e.g., EGFRvIII CAR-expressing cell) comprises up to about 1×10⁷, 2×10⁷, 5×10⁷, 1×10⁸, 2×10⁸, 3×10⁸, 5×10⁸, 1×10⁹, 2×10⁹, or 5×10⁹ cells.

In some embodiments, a dose of CAR cells (e.g., EGFRvIII CAR-expressing cell) comprises up to about 1×10⁷, 1.5×10⁷, 2×10⁷, 2.5×10⁷, 3×10⁷, 3.5×10⁷, 4×10⁷, 5×10⁷, 1×10⁸, 1.5×10⁸, 2×10⁸, 2.5×10⁸, 3×10⁸, 3.5×10⁸, 4×10⁸, 5×10⁸, 1×10⁹, 2×10⁹, or 5×10⁹ cells. In some embodiments, a dose of CAR cells (e.g., EGFRvIII CAR-expressing cell) comprises up to about 1.75-5×10⁸ cells per infusion, e.g., about 2×10⁸ cells per infusion or about 5×10⁸ cells per infusion. In some embodiments, the subject is administered about about 1.75-5×10⁸ cells per infusion of EGFRvIII CAR-expressing cells. In some embodiments, the subject is administered about 2×10⁸ cells per infusion of EGFRvIII CAR-expressing cells. In some embodiments, the subject is administered about 5×10⁸ cells per infusion of EGFRvIII CAR-expressing cells.

In some embodiments, a dose of CAR-expressing cells (e.g., CAR-expressing cells described herein, e.g., EGFRvIII CAR-expressing cells described herein) comprises about 1×10⁶ cells/m² to about 1×10⁹ cells/m², e.g., about 1×10⁷ cells/m² to about 5×10⁸ cells/m², e.g., about 1.5×10⁷ cells/m², about 2×10⁷ cells/m², about 4.5×10⁷ cells/m², about 10⁸ cells/m², about 1.2×10⁸ cells/m², or about 2×10⁸ cells/m².

In embodiments, the EGFRvIII CAR-expressing cells are administered in a plurality of doses, e.g., a first dose, a second dose, and optionally a third dose. In embodiments, the method comprises treating a subject (e.g., an adult subject) having a cancer (e.g., GBM), comprising administering to the subject a first dose, a second dose, and optionally one or more additional doses, each dose comprising immune effector cells expressing a CAR molecule, e.g., an EGFRvIII CAR molecule, e.g., a CAR molecule according to SEQ ID NO: 108.

In embodiments, the method comprises administering a dose of 2-5×10⁶ viable CAR-expressing cells/kg, wherein the subject has a body mass of less than 50 kg; or

administering a dose of 1.0-2.5×10⁸ viable CAR-expressing cells, wherein the subject has a body mass of at least 50 kg.

In embodiments, a single dose is administered to the subject, e.g., a pediatric or an adult subject.

In embodiments, the doses are administered on sequential days, e.g., the first dose is administered on day 1, the second dose is administered on day 2, and the optional third dose (if administered) is administered on day 3.

In embodiments, a fourth, fifth, or sixth dose, or more doses, are administered.

In embodiments, the first dose comprises about 10% of the total dose, the second dose comprises about 30% of the total dose, and the third dose comprises about 60% of the total dose, wherein the aforementioned percentages have a sum of 100%. In embodiments, the first dose comprises about 9-11%, 8-12%, 7-13%, or 5-15% of the total dose. In embodiments, the second dose comprises about 29-31%, 28-32%, 27-33%, 26-34%, 25-35%, 24-36%, 23-37%, 22-38%, 21-39%, or 20-40% of the total dose. In embodiments, the third dose comprises about 55-65%, 50-70%, 45-75%, or 40-80% of the total dose. In embodiments, the total dose refers to the total number of viable CAR-expressing cells administered over the course of 1 week, 2 weeks, 3 weeks, or 4 weeks. In some embodiments wherein two doses are administered, the total dose refers to the sum of the number of viable CAR-expressing cells administered to the subject in the first and second doses. In some embodiments wherein three doses are administered, the total dose refers to the sum of the number of viable CAR-expressing cells administered to the subject in the first, second, and third doses.

In embodiments, the dose is measured according to the number of viable CAR-expressing cells therein. CAR expression can be measured, e.g., by flow cytometry using an antibody molecule that binds the CAR molecule and a detectable label. Viability can be measured, e.g., by Cellometer.

In embodiments, the viable CAR-expressing cells are administered in ascending doses. In embodiments, the second dose is larger than the first dose, e.g., larger by 10%, 20%, 30%, or 50%. In embodiments, the second dose is twice, three times, four times, or five times the size of the first dose. In embodiments, the third dose is larger than the second dose, e.g., larger by 10%, 20%, 30%, or 50%. In embodiments, the third dose is twice, three times, four times, or five times the size of the second dose.

In certain embodiments, the method includes one, two, three, four, five, six, seven or all of a)-h) of the following:

a) the number of CAR-expressing, viable cells administered in the first dose is no more than 1/3, of the number of CAR-expressing, viable cells administered in the second dose;

b) the number of CAR-expressing, viable cells administered in the first dose is no more than 1/X, wherein X is 2, 3, 4, 5, 6, 7, 8, 9, 10, 15, 20, 30, 40 or 50, of the total number of CAR-expressing, viable cells administered;

c) the number of CAR-expressing, viable cells administered in the first dose is no more than 1×10⁷, 2×10⁷, 3×10⁷, 4×10⁷, 5×10⁷, 6×10⁷, 7×10⁷, 8×10⁷, 9×10⁷, 1×10⁸, 1.75×10⁸, 2×10⁸, 3×10⁸, 4×10⁸, or 5×10⁸ CAR-expressing, viable cells, and the second dose is greater than the first dose;

d) the number of CAR-expressing, viable cells administered in the second dose is no more than 1/2, of the number of CAR-expressing, viable cells administered in the third dose;

e) the number of CAR-expressing, viable cells administered in the second dose is no more than 1/Y, wherein Y is 2, 3, 4, 5, 6, 7, 8, 9, 10, 15, 20, 30, 40 or 50, of the total number of CAR-expressing, viable cells administered;

f) the number of CAR-expressing, viable cells administered in the second dose is no more than 1×10⁷, 2×10⁷, 3×10⁷, 4×10⁷, 5×10⁷, 6×10⁷, 7×10⁷, 8×10⁷, 9×10⁷, 1×10⁸, 1.75×10⁸, 2×10⁸, 3×10⁸, 4×10⁸, or 5×10⁸ CAR-expressing, viable cells, and the third dose is greater than the second dose;

h) the dosages and time periods of administration of the first, second, and optionally third doses are selected such that the subject experiences CRS at a level no greater than 4, 3, 2, or 1.

In embodiments, the total dose is about 5×10⁸ CAR-expressing, viable cells. In embodiments, the total dose is about 5×10⁷-5×10⁸ CAR-expressing, viable cells. In embodiments, the first dose is about 5×10⁷ (e.g., ±10%, 20%, or 30%) CAR-expressing, viable cells, the second dose is about 1.5×10⁸ (e.g., ±10%, 20%, or 30%) CAR-expressing, viable cells, and the third dose is about 3×10⁸ (e.g., ±10%, 20%, or 30%) CAR-expressing, viable cells.

In embodiments, the subject is evaluated for CRS after receiving a dose, e.g., after receiving the first dose, the second dose, and/or the third dose.

In embodiments, the subject receives a CRS treatment, e.g., tocilizumab, a corticosteroid, etanercept, or siltuximab. In embodiments, the CRS treatment is administered before or after the first dose of cells comprising the CAR molecule. In embodiments, the CRS treatment is administered before or after the second dose of cells comprising the CAR molecule. In embodiments, the CRS treatment is administered before or after the third dose of cells comprising the CAR molecule. In embodiments, the CRS treatment is administered between the first and second doses of cells comprising the CAR molecule, and/or between the second and third doses of cells comprising the CAR molecule.

In embodiments, in a subject having CRS after the first dose, e.g., CRS grade 1, 2, 3, or 4, the second dose is administered at least 2, 3, 4, or 5 days after the first dose. In embodiments, in a subject having CRS after the second dose, e.g., CRS grade 1, 2, 3, or 4, the third dose is administered at least 2, 3, 4, or 5 days after the second dose. In embodiments, in a subject having CRS after the first dose, the second dose of CAR-expressing cells is delayed relative to when the second dose would have been administered had the subject not had CRS. In embodiments, in a subject having CRS after the second dose, the third dose of CAR-expressing cells is delayed relative to when the third dose would have been administered had the subject not had CRS.

In embodiments, the subject has a cancer with a high disease burden before the first dose is administered. In embodiments, the subject has bone marrow blast levels of at least 1%, 2%, 3%, 4%, 5%, 6%, 7%, 8%, 9%, 10%, 15%, 20%, 25%, 30%, 35%, 40%, 45%, or 50%, e.g., at least 5%. In embodiments, the subject has a cancer in stage I, II, III, or IV. In embodiments, the subject has a tumor mass of at least 1, 2, 5, 10, 20, 50, 100, 200, 500, or 1000 g, e.g., in a single tumor or a plurality of tumors.

In some embodiments, the subject has cancer (e.g., a solid cancer described herein). In an embodiment, the subject has GBM.

In one embodiment, the cancer is a disease associated with EGFRvIII expression, e.g., as described herein. In other embodiments, the cancer is a disease associated with a tumor antigen, e.g., as described herein. In embodiments, the CAR molecule is a CAR molecule as described herein.

In one aspect, CAR-expressing cells, e.g., EGFRvIII CAR-expressing cells, are generated using lentiviral viral vectors, such as lentivirus. CAR-expressing cells generated that way will have stable CAR expression.

In one aspect, the CAR-expressing cells transiently express CAR vectors for 4, 5, 6, 7, 8, 9, 10, 11, 12, 13, 14, 15 days after transduction. Transient expression of CARs can be effected by RNA CAR vector delivery. In one aspect, the CAR RNA is transduced into the T cell by electroporation.

A potential issue that can arise in patients being treated using transiently expressing CAR-expressing cells (particularly with murine scFv bearing CAR-expressing cells) is anaphylaxis after multiple treatments.

Without being bound by this theory, it is believed that such an anaphylactic response might be caused by a patient developing humoral anti-CAR response, i.e., anti-CAR antibodies having an anti-IgE isotype. It is thought that a patient's antibody producing cells undergo a class switch from IgG isotype (that does not cause anaphylaxis) to IgE isotype when there is a ten to fourteen day break in exposure to antigen.

If a patient is at high risk of generating an anti-CAR antibody response during the course of transient CAR therapy (such as those generated by RNA transductions), CAR-expressing cell infusion breaks should not last more than ten to fourteen days.

Using CARs with human (instead of murine) scFvs can reduce the likelihood and intensity of a patient having an anti-CAR response.

Dosages and therapeutic regimens of the PD-1 inhibitor, e.g., anti-PD-1 antibody molecule, can be determined by a skilled artisan. Suitable dosages of the molecules used will depend on the age and weight of the subject and the particular drug used.

Methods of administering the antibody molecules are known in the art and are described below. Suitable dosages of the molecules used will depend on the age and weight of the subject and the particular drug used. Dosages and therapeutic regimens of the anti-PD-1 antibody molecule can be determined by a skilled artisan.

In certain embodiments, the anti-PD-1 antibody molecule is administered by injection (e.g., subcutaneously or intravenously) at a dose of about 1 to 30 mg/kg, e.g., about 5 to 25 mg/kg, about 10 to 20 mg/kg, about 1 to 5 mg/kg, or about 3 mg/kg. In some embodiments, the anti-PD-1 antibody molecule is administered at a dose of about 1 mg/kg, about 3 mg/kg, about 5 mg/kg, about 10 mg/kg, about 20 mg/kg, about 30 mg/kg, or about 40 mg/kg. In some embodiments, the anti-PD-1 antibody molecule is administered at a dose of about 1-3 mg/kg, or about 3-10 mg/kg. In some embodiments, the anti-PD-1 antibody molecule is administered at a dose of about 0.5-2, 2-4, 2-5, 5-15, or 5-20 mg/kg. The dosing schedule can vary from e.g., once a week to once every 2, 3, or 4 weeks. In one embodiment, the anti-PD-1 antibody molecule is administered at a dose from about 10 to 20 mg/kg every other week. In another embodiment, the anti-PD-1 antibody molecule is administered at a dose of about 1 mg/kg once every two weeks, about 3 mg/kg once every two weeks, 10 mg/kg once every two weeks, 3 mg/kg once every four weeks, or 5 mg/kg once every four weeks.

In other embodiments, the anti-PD-1 antibody molecule is administered by injection (e.g., subcutaneously or intravenously) at a dose (e.g., a flat dose) of about 200 mg to 500 mg, e.g., about 250 mg to 450 mg, about 300 mg to 400 mg, about 250 mg to 350 mg, about 350 mg to 450 mg, or about 200 mg, about 300 mg, or about 400 mg. In some embodiments, the anti-PD-1 antibody molecule is administered at a dose of about 200 mg, about 250 mg, about 300 mg, about 350 mg, about 400 mg, about 450 mg, or about 500 mg. In some embodiments, the anti-PD1 antibody is administered at a dose of 200 or 300 mg. In some embodiments, the anti-PD-1 antibody molecule is administered at a dose of about 250-450 mg, or about 300-400 mg. In some embodiments, the anti-PD-1 antibody molecule is administered at a dose of about 200-300 mg, 250-350 mg, 300-400 mg, 350-450 mg, or 400-500 mg. The dosing schedule can vary from e.g., once a week to once every 2, 3, 4, 5, or 6 weeks. In one embodiment the anti-PD-1 antibody molecule is administered at a dose from about 300 mg to 400 mg once every three or once every four weeks. In one embodiment, the anti-PD-1 antibody molecule is administered at a dose from about 200 mg once every three weeks. In one embodiment, the anti-PD-1 antibody molecule is administered at a dose from about 300 mg once every three weeks. In one embodiment, the anti-PD-1 antibody molecule is administered at a dose from about 400 mg once every four weeks. The anti-PD-1 antibody can be administered one or more times, e.g., one, two, three, four, five, six, seven or more times. In one embodiment, the anti-PD-1 antibody is administered six times. The anti-PD-1 antibody can be administered at least 5 days, e.g., about 5, 6, 7, 8, 9, 10, 11, 12, 13, 14, 20, 25, 30, 35, or 40 days, after administration of CAR-expressing cells, e.g., EGFRvIII CAR expressing cells. In some embodiments, the anti-PD-1 antibody can be administered about 8 days or about 15 days after administration of CAR-expressing cells, e.g., EGFRvIII CAR expressing cells.

The antibody molecules can be administered by a variety of methods known in the art, although for many therapeutic applications, the preferred route/mode of administration is intravenous injection or infusion. For example, the antibody molecules can be administered by intravenous infusion at a rate of more than 20 mg/min, e.g., 20-40 mg/min, and typically greater than or equal to 40 mg/min to reach a dose of about 35 to 440 mg/m², typically about 70 to 310 mg/m², and more typically, about 110 to 130 mg/m². In embodiments, the antibody molecules can be administered by intravenous infusion at a rate of less than 10 mg/min; preferably less than or equal to 5 mg/min to reach a dose of about 1 to 100 mg/m², preferably about 5 to 50 mg/m², about 7 to 25 mg/m² and more preferably, about 10 mg/m². As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

The antibody molecule can be administered by intravenous infusion at a rate of more than 20 mg/min, e.g., 20-40 mg/min, and typically greater than or equal to 40 mg/min to reach a dose of about 35 to 440 mg/m², typically about 70 to 310 mg/m², and more typically, about 110 to 130 mg/m². In embodiments, the infusion rate of about 110 to 130 mg/m² achieves a level of about 3 mg/kg. In other embodiments, the antibody molecule can be administered by intravenous infusion at a rate of less than 10 mg/min, e.g., less than or equal to 5 mg/min to reach a dose of about 1 to 100 mg/m², e.g., about 5 to 50 mg/m², about 7 to 25 mg/m², or, about 10 mg/m². In some embodiments, the antibody is infused over a period of about 30 min.

In an embodiment, the method comprises administering radiation to the subject, e.g. prior to administration of the CAR-expressing cell. In embodiments, the total dose of radiation does not exceed a standard dose, e.g. 60 Gy. In embodiments, the total dose of radiation does not exceed 40 Gy. In embodiments, the radiation is administered in one or more fractions, e.g. 2, 3, 4, 5, 6, 7, 8, 9, 10, 11, 12, 13, 14, 15, 16, 17, 18, 19, 20, 21, 22, 23, 24, 25, 26, 27, 28, 29, 30, or more fractions. For example, a total dose of 60 Gy may be delivered in 30 equivalent fractions of 2 Gy each, and a total dose of 40 Gy may be delivered in 15 equivalent fractions of 8/3 Gy each.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1: Generation of EGFRvIII CAR T Cells

Murine monoclonal antibody (mAb) 3C10 was originally developed by immunization of mice with a 14 amino acid peptide (PEP3) including the EGFRvIII-specific fusion junction and demonstrated highly specific recognition of EGFRvIII without any detectable binding to wild-type EGFR (Okamoto et al, British J. Cancer 1996, 73:1366-1372). Subsequently, a single-chain variable fragment (scFv) of mAb 3C10 was produced and cDNA for the 3C10 scFv was obtained. While avidity and/or antigen-specificity of the original mAbs can be often lost in scFv forms, the 3C10 scFv retained its selective reactivity with the EGFRvIII-specific epitope (Nakayashiki et al., Jpn. J. Cancer Res. 2000, 91:1035-1043).

An EGFRvIII CAR was constructed by cloning the 3C10scFv (mouse) with CD28, 4-1BB, and CD3 zeta into the pELNS lentiviral backbone plasmid (EF1 promoter). Another EGFRvIII CAR was generated by cloning the 3C10scFv into a CD8ahinge/CD8TM/4-1BB/CD3zeta pELNS lentiviral backbone, which is expressed by EF1a promoter.

3C10scFv-CD28BBzeta CAR (Amino Acid)
(SEQ ID NO: 1)
MALPVTALLLPLALLLHAARPGSEIQLQQSGAELVKPGASVKLSCTGSGFNIEDY
YIHWVKQRTEQGLEWIGRIDPENDETKYGPIFQGRATITADTSSNTVYLQLSSLTS
EDTAVYYCAFRGGVYWGPGTTLTVSSGGGGSGGGGSGGGGSHMDVVMTQSPL
TLSVAIGQSASISCKSSQSLLDSDGKTYLNWLLQRPGQSPKRLISLVSKLDSGVPDRFTGS
GSGTDFTLRISRVEAEDLGIYYCWQGTHFPGTFGGGTKLEIKASTTTPAPR
PPTPAPTIASQPLSLRPEACRPAAGGAVHTRGLDFACDFWVLVVVGGVLACYSL
LVTVAFIIFWVRSKRSRLLHSDYMNMTPRRPGPTRKHYQPYAPPRDFAAYRSKR
GRKKLLYIFKQPFMRPVQTTQEEDGCSCRFPEEEEGGCELRVKFSRSADAPAYKQ
GQNQLYNELNLGRREEYDVLDKRRGRDPEMGGKPRRKNPQEGLYNELQKDKMAEAY
SEIGMKGERRRGKGHDGLYQGLSTATKDTYDALHMQALPPR
3C10scFv-BBz CAR (Amino Acid)
(SEQ ID NO: 2)
MALPVTALLLPLALLLHAARPGSEIQLQQSGAELVKPGASVKLSCTGSGFNIE
DYYIHWVKQRTEQGLEWIGRIDPENDETKYGPIFQGRATITADTSSNTVYLQLSSLTSED
TAVYYCAFRGGVYWGPGTTLTVSSGGGGSGGGGSGGGGSHMDVVMTQSPL
TLSVAIGQSASISCKSSQSLLDSDGKTYLNWLLQRPGQSPKRLISLVSKLDSGVPD
RFTGSGSGTDFTLRISRVEAEDLGIYYCWQGTHFPGTFGGGTKLEIKASTTTPAPR
PPTPAPTIASQPLSLRPEACRPAAGGAVHTRGLDFACDIYIWAPLAGTCGVLLLSL
VITLYCKRGRKKLLYIFKQPFMRPVQTTQEEDGCSCRFPEEEEGGCELRVKFSRSADAPA
YKQGQNQLYNELNLGRREEYDVLDKRRGRDPEMGGKPRRKNPQEGLYNELQKDKMA
EAYSEIGMKGERRRGKGHDGLYQGLSTATKDTYDALHMQALPPR
3C10scFv-CD28BBzeta CAR (Nucleic Acid)
(SEQ ID NO: 18)
atggccttaccagtgaccgccttgctcctgccgctggccttgctgctccacgccgccaggccgggatccgagattcagctgcag
caatctggggcagaacttgtgaagccaggggcctcagtcaagctgtcctgcacaggttctggcttcaacattgaagactactatat
tcactgggtgaagcagaggactgaacagggcctggaatggattggaaggattgatcctgagaatgatgaaactaaatatggccc
aatattccagggcagggccactataacagcagacacatcctccaacacagtctacctgcaactcagcagcctgacatctgagga
cactgccgtctattactgtgcctttcgcggtggagtctactgggggccaggaaccactctcacagtctcctcaggaggtggtggttccggtgg
tggtggttccggaggtggtggttcacatatggatgttgtgatgacccagtctccactcactctatcggttgccattggac
aatcagcctccatctcttgcaagtcaagtcagagcctcttagatagtgatggaaagacatatttgaattggttgttacagaggccaggccag
tctccaaagcgcctaatctctctggtgtctaaactggactctggagtccctgacaggttcactggcagtggatcagggac
agatttcacactgagaatcagcagagtggaggctgaggatttgggaatttattattgctggcaaggtacacattttcctgggacgtt
cggtggagggaccaagctggagataaaagctagcaccacgacgccagcgccgcgaccaccaacaccggcgcccaccatcg
cgtcgcagcccctgtccctgcgcccagaggcgtgccggccagcggcggggggcgcagtgcacacgagggggctggacttcgcctgtg
atttttgggtgctggtggtggttggtggagtcctggcttgctatagcttgctagtaacagtggcctttattattttctgggtg
aggagtaagaggagcaggctcctgcacagtgactacatgaacatgactccccgccgccccgggcccacccgcaagcattacc
agccctatgccccaccacgcgacttcgcagcctatcgctccaaacggggcagaaagaaactcctgtatatattcaaacaaccatt
tatgagaccagtacaaactactcaagaggaagatggctgtagctgccgatttccagaagaagaagaaggaggatgtgaactga
gagtgaagttcagcaggagcgcagacgcccccgcgtacaagcagggccagaaccagctctataacgagctcaatctaggacgaagaga
ggagtacgatgttttggacaagagacgtggccgggaccctgagatggggggaaagccgagaaggaagaaccct
caggaaggcctgtacaatgaactgcagaaagataagatggcggaggcctacagtgagattgggatgaaaggcgagcgccgg
aggggcaaggggcacgatggcctttaccagggtctcagtacagccaccaaggacacctacgacgcccttcacatgcaggccc
tgccccctcgc
3C10scFv-BBz CAR (Nucleic Acid)
(SEQ ID NO: 19)
Atggccttaccagtgaccgccttgctcctgccgctggccttgctgctccacgccgccaggccgggatccgagattcagctgca
gcaatctggggcagaacttgtgaagccaggggcctcagtcaagctgtcctgcacaggttctggcttcaacattgaagactactat
attcactgggtgaagcagaggactgaacagggcctggaatggattggaaggattgatcctgagaatgatgaaactaaatatggc
ccaatattccagggcagggccactataacagcagacacatcctccaacacagtctacctgcaactcagcagcctgacatctgaggacactg
ccgtctattactgtgcctttcgcggtggagtctactgggggccaggaaccactctcacagtctcctcaggaggtggtg
gttccggtggtggtggttccggaggtggtggttcacatatggatgttgtgatgacccagtctccactcactctatcggttgccattg
gacaatcagcctccatctcttgcaagtcaagtcagagcctcttagatagtgatggaaagacatatttgaattggttgttacagaggc
caggccagtctccaaagcgcctaatctctctggtgtctaaactggactctggagtccctgacaggttcactggcagtggatcagg
gacagatttcacactgagaatcagcagagtggaggctgaggatttgggaatttattattgctggcaaggtacacattttcctgggacgttcg
gtggagggaccaagctggagataaaagctagcaccacgacgccagcgccgcgaccaccaacaccggcgcccacc
atcgcgtcgcagcccctgtccctgcgcccagaggcgtgccggccagcggcggggggcgcagtgcacacgagggggctgg
acttcgcctgtgatatctacatctgggcgcccttggccgggacttgtggggtccttctcctgtcactggttatcaccctttactgcaa
acggggcagaaagaaactcctgtatatattcaaacaaccatttatgagaccagtacaaactactcaagaggaagatggctgtagc
tgccgatttccagaagaagaagaaggaggatgtgaactgagagtgaagttcagcaggagcgcagacgcccccgcgtacaagcagggcc
agaaccagctctataacgagctcaatctaggacgaagagaggagtacgatgttttggacaagagacgtggccggg
accctgagatggggggaaagccgagaaggaagaaccctcaggaaggcctgtacaatgaactgcagaaagataagatggcggaggcct
acagtgagattgggatgaaaggcgagcgccggaggggcaaggggcacgatggcctttaccagggtctcagtacagccaccaaggaca
cctacgacgcccttcacatgcaggccctgccccctcgc

The scFv fragment termed “139” is a human antibody to EGFRvIII (Morgan et al., 2012 Hum Gene Ther 23(10): 1043-53). An EGFRvIII CAR comprising the 139 scFv was generated by initially synthesizing the 139 scFv. The sequence for the 139 scFv was cloned with a leader sequence, CD8 hinge, transmembrane (TM) domain, and the desired signaling domains. For example, the sequence for the 139 scFv was cloned with the signaling domains for 4-1BB and CD3 zeta. The CAR construct (139scFv-BBZ) is expressed from the pELNS vector for lentivirus production.

139scFv-BBz CAR (Amino Acid)
(SEQ ID NO: 3)
MALPVTALLLPLALLLHAARPGSDIQMTQSPSSLSASVGDRVTITCRASQGIRNNL
AWYQQKPGKAPKRLIYAASNLQSGVPSRFTGSGSGTEFTLIVSSLQPEDFATYYC
LQHHSYPLTSGGGTKVEIKRTGSTSGSGKPGSGEGSEVQVLESGGGLVQPGGSLR
LSCAASGFTFSSYAMSWVRQAPGKGLEWVSAISGSGGSTNYADSVKGRFTISRD
NSKNTLYLQMNSLRAEDTAVYYCAGSSGWSEYWGQGTLVTVSSASTTTPAPRP
PTPAPTIASQPLSLRPEACRPAAGGAVHTRGLDFACDIYIWAPLAGTCGVLLLSLV
ITLYCKRGRKKLLYIFKQPFMRPVQTTQEEDGCSCRFPEEEEGGCELRVKFSRSADAPAY
KQGQNQLYNELNLGRREEYDVLDKRRGRDPEMGGKPRRKNPQEGLYNELQKDKMAE
AYSEIGMKGERRRGKGHDGLYQGLSTATKDTYDALHMQALPPR
139scFv-BBz CAR (Nucleic Acid)
(SEQ ID NO: 20)
Atggccttaccagtgaccgccttgctcctgccgctggccttgctgctccacgccgccaggccgggatccgacatccagatgacccagagc
cctagcagcctgagcgccagcgtgggcgacagagtgaccatcacctgtcgggccagccagggcatcagaaaca
acctggcctggtatcagcagaagcccggcaaggcccccaagagactgatctacgctgccagcaatctgcagagcggcgtgcc
cagcagattcaccggaagcggctccggcaccgagttcaccctgatcgtgtccagcctgcagcccgaggacttcgccacctact
actgcctgcagcaccacagctaccctctgaccagcggcggaggcaccaaggtggagatcaagcggaccggcagcaccagc
ggcagcggcaagcctggcagcggcgagggaagcgaggtccaggtgctggaatctggcggcggactggtgcagcctggcggcagcct
gagactgagctgtgccgccagcggcttcaccttcagcagctacgccatgtcttgggtccggcaggctcctggaaag
ggcctggaatgggtgtccgccatcagcggctctggcggctccaccaactacgccgacagcgtgaagggccggttcaccatcagccggg
acaacagcaagaacaccctgtatctgcagatgaacagcctgagagccgaggacaccgccgtgtactactgtgccgg
cagcagcgggtggagcgagtactggggccagggcacactggtcacagtgtctagcgctagcaccacgacgccagcgccgc
gaccaccaacaccggcgcccaccatcgcgtcgcagcccctgtccctgcgcccagaggcgtgccggccagcggcgggggg
cgcagtgcacacgagggggctggacttcgcctgtgatatctacatctgggcgcccttggccgggacttgtggggtccttctcctgtcactgg
ttatcaccctttactgcaaacggggcagaaagaaactcctgtatatattcaaacaaccatttatgagaccagtacaaactactcaagaggaag
atggctgtagctgccgatttccagaagaagaagaaggaggatgtgaactgagagtgaagttcagcaggagcgcagacgcccccgcgtac
aagcagggccagaaccagctctataacgagctcaatctaggacgaagagaggagtacgatgttttggacaagagacgtggccgggaccc
tgagatggggggaaagccgagaaggaagaaccctcaggaaggcctgtacaatgaactgcagaaagataagatggcggaggcctacagt
gagattgggatgaaaggcgagcgccggaggggcaaggggcacgatggcctttaccagggtctcagtacagccaccaaggacacctac
gacgcccttcacatgcaggccctgccccctcgct

### Car Components

### Nucleic Acid Sequences:

3C10 scFv Nucleotide Sequence (Mouse);
(SEQ ID NO: 4)
GAGATTCAGCTGCAGCAATCTGGGGCAGAACTTGTGAAGCCAGGGGCCTC
AGTCAAGCTGTCCTGCACAGGTTCTGGCTTCAACATTGAAGACTACTATA
TTCACTGGGTGAAGCAGAGGACTGAACAGGGCCTGGAATGGATTGGAAGG
ATTGATCCTGAGAATGATGAAACTAAATATGGCCCAATATTCCAGGGCAG
GGCCACTATAACAGCAGACACATCCTCCAACACAGTCTACCTGCAACTCA
GCAGCCTGACATCTGAGGACACTGCCGTCTATTACTGTGCCTTTCGCGGT
GGAGTCTACTGGGGGCCAGGAACCACTCTCACAGTCTCCTCAGGAGGTGG
TGGTTCCGGTGGTGGTGGTTCCGGAGGTGGTGGTTCACATATGGATGTTG
TGATGACCCAGTCTCCACTCACTCTATCGGTTGCCATTGGACAATCAGCC
TCCATCTCTTGCAAGTCAAGTCAGAGCCTCTTAGATAGTGATGGAAAGAC
ATATTTGAATTGGTTGTTACAGAGGCCAGGCCAGTCTCCAAAGCGCCTAA
TCTCTCTGGTGTCTAAACTGGACTCTGGAGTCCCTGACAGGTTCACTGGC
AGTGGATCAGGGACAGATTTCACACTGAGAATCAGCAGAGTGGAGGCTGA
GGATTTGGGAATTTATTATTGCTGGCAAGGTACACATTTTCCTGGGACGT
TCGGTGGAGGGACCAAGCTGGAGATAAAA
139 scFv Nucleotide Sequence (Humanized);
(SEQ ID NO: 5)
GACATCCAGATGACCCAGAGCCCTAGCAGCCTGAGCGCCAGCGTGGGCGA
CAGAGTGACCATCACCTGTCGGGCCAGCCAGGGCATCAGAAACAACCTGG
CCTGGTATCAGCAGAAGCCCGGCAAGGCCCCCAAGAGACTGATCTACGCT
GCCAGCAATCTGCAGAGCGGCGTGCCCAGCAGATTCACCGGAAGCGGCTC
CGGCACCGAGTTCACCCTGATCGTGTCCAGCCTGCAGCCCGAGGACTTCG
CCACCTACTACTGCCTGCAGCACCACAGCTACCCTCTGACCAGCGGCGGA
GGCACCAAGGTGGAGATCAAGCGGACCGGCAGCACCAGCGGCAGCGGCAA
GCCTGGCAGCGGCGAGGGAAGCGAGGTCCAGGTGCTGGAATCTGGCGGCG
GACTGGTGCAGCCTGGCGGCAGCCTGAGACTGAGCTGTGCCGCCAGCGGC
TTCACCTTCAGCAGCTACGCCATGTCTTGGGTCCGGCAGGCTCCTGGAAA
GGGCCTGGAATGGGTGTCCGCCATCAGCGGCTCTGGCGGCTCCACCAACT
ACGCCGACAGCGTGAAGGGCCGGTTCACCATCAGCCGGGACAACAGCAAG
AACACCCTGTATCTGCAGATGAACAGCCTGAGAGCCGAGGACACCGCCGT
GTACTACTGTGCCGGCAGCAGCGGGTGGAGCGAGTACTGGGGCCAGGGCA
CACTGGTCACAGTGTCTAGC
leader (nucleic acid sequence);
(SEQ ID NO: 6)
ATGGCCTTACCAGTGACCGCCTTGCTCCTGCCGCTGGCCTTGCTGCTCCA
CGCCGCCAGGCCG
hinge (nucleic acid sequence);
(SEQ ID NO: 7)
ACCACGACGCCAGCGCCGCGACCACCAACACCGGCGCCCACCATCGCGTC
GCAGCCCCTGTCCCTGCGCCCAGAGGCGTGCCGGCCAGCGGCGGGGGGCG
CAGTGCACACGAGGGGGCTGGACTTCGCCTGTGAT
transmembrane (nucleic acid sequence);
(SEQ ID NO: 8)
ATCTACATCTGGGCGCCCTTGGCCGGGACTTGTGGGGTCCTTCTCCTGTC
ACTGGTTATCACCCTTTACTGC
4-1BB Intracellular domain
(nucleic acid sequence);
(SEQ ID NO: 9)
AAACGGGGCAGAAAGAAACTCCTGTATATATTCAAACAACCATTTATGAG
ACCAGTACAAACTACTCAAGAGGAAGATGGCTGTAGCTGCCGATTTCCAG
AAGAAGAAGAAGGAGGATGTGAACTG
CD3 zeta (nucleic acid sequence);
(SEQ ID NO: 10)
AGAGTGAAGTTCAGCAGGAGCGCAGACGCCCCCGCGTACAAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGAGA
AGGAAGAACCCTCAGGAAGGCCTGTACAATGAACTGCAGAAAGATAAGAT
GGCGGAGGCCTACAGTGAGATTGGGATGAAAGGCGAGCGCCGGAGGGGCA
AGGGGCACGATGGCCTTTACCAGGGTCTCAGTACAGCCACCAAGGACACC
TACGACGCCCTTCACATGCAGGCCCTGCCCCCTCGC
CD3 zeta (nucleic acid sequence;
NCBI Reference Sequence NM_000734.3);
(SEQ ID NO: 100)
AGAGTGAAGTTCAGCAGGAGCGCAGACGCCCCCGCGTACCAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGAGA
AGGAAGAACCCTCAGGAAGGCCTGTACAATGAACTGCAGAAAGATAAGAT
GGCGGAGGCCTACAGTGAGATTGGGATGAAAGGCGAGCGCCGGAGGGGCA
AGGGGCACGATGGCCTTTACCAGGGTCTCAGTACAGCCACCAAGGACACC
TACGACGCCCTTCACATGCAGGCCCTGCCCCCTCGC

### Amino Acid Sequences:

3C10 scFv Amino Sequence (Mouse);
(SEQ ID NO: 11)
EIQLQQSGAELVKPGASVKLSCTGSGFNIEDYYIHWVKQRTEQGLEWIGR
IDPENDETKYGPIFQGRATITADTSSNTVYLQLSSLTSEDTAVYYCAFRG
GVYWGPGTTLTVSSGGGGSGGGGSGGGGSHMDVVMTQSPLTLSVAIGQSA
SISCKSSQSLLDSDGKTYLNWLLQRPGQSPKRLISLVSKLDSGVPDRFTG
SGSGTDFTLRISRVEAEDLGIYYCWQGTHFPGTFGGGTKLEIK
139 scFv Amino Sequence (Human);
(SEQ ID NO: 12)
DIQMTQSPSSLSASVGDRVTITCRASQGIRNNLAWYQQKPGKAPKRLIYA
ASNLQSGVPSRFTGSGSGTEFTLIVSSLQPEDFATYYCLQHHSYPLTSGG
GTKVEIKRTGSTSGSGKPGSGEGSEVQVLESGGGLVQPGGSLRLSCAASG
FTFSSYAMSWVRQAPGKGLEWVSAISGSGGSTNYADSVKGRFTISRDNSK
NTLYLQMNSLRAEDTAVYYCAGSSGWSEYWGQGTLVTVSS
leader (amino acid sequence)
(SEQ ID NO: 13)
MALPVTALLLPLALLLHAARP
hinge (amino acid sequence)
(SEQ ID NO: 14)
TTTPAPRPPTPAPTIASQPLSLRPEACRPAAGGAVHTRGLDFACD
transmembrane (amino acid sequence)
(SEQ ID NO: 15)
IYIWAPLAGTCGVLLLSLVITLYC
4-1BB Intracellular domain (amino acid sequence)
(SEQ ID NO: 16)
KRGRKKLLYIFKQPFMRPVQTTQEEDGCSCRFPEEEEGGCEL
CD3 zeta domain (amino acid sequence)
(SEQ ID NO: 17)
RVKFSRSADAPAYKQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR
CD3 zeta domain (amino acid sequence;
NCBI Reference Sequence NM_000734.3)
(SEQ ID NO: 99)
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

The nucleotide encoding the polypeptide of SEQ ID NO:11 is provided as SEQ ID NO:4. The nucleotide encoding the polypeptide of SEQ ID NO:12 is provided as SEQ ID NO:5. The nucleotide encoding the polypeptide of SEQ ID NO:13 is provided as SEQ ID NO:6. The nucleotide encoding the polypeptide of SEQ ID NO:14 is provided as SEQ ID NO:7. The nucleotide encoding the polypeptide of SEQ ID NO:15 is provided as SEQ ID NO:8. The nucleotide encoding the polypeptide of SEQ ID NO:16 is provided as SEQ ID NO:9. The nucleotide encoding the polypeptide of SEQ ID NO:17 is provided as SEQ ID NO:10. The nucleotide encoding the polypeptide of SEQ ID NO:1 is provided as SEQ ID NO:18. The nucleotide encoding the polypeptide of SEQ ID NO:2 is provided as SEQ ID NO:19. The nucleotide encoding the polypeptide of SEQ ID NO:3 is provided as SEQ ID NO:20. The nucleotide encoding the polypeptide of SEQ ID NO:99 is provided as SEQ ID NO:100.

The predicted CDR designations for the EGFRvIII CAR under Kabat are as follows:

VH:
(SEQ ID NO: 21);
EIQLQQSGAELVKPGASVKLSCTGSGFNIEDYYIHWVKQRTEQGLEWIGR
IDPENDETKYGPIFQGRATITADTSSNTVYLQLSSLTSEDTAVYYCAFRG
GVYWGPGTTLTVSS;
(SEQ ID NO: 22)
wherein CDR1 is DYYIH,
(SEQ ID NO: 23)
CDR2 is RIDPENDETKYGPIFQG,
and
(SEQ ID NO: 24)
CDR3 is RGGVY.
VL:
(SEQ ID NO: 25);
DVVMTQSPLTLSVAIGQSASISCKSSQSLLDSDGKTYLNWLLQRPGQSPK
RLISLVSKLDSGVPDRFTGSGSGTDFTLRISRVEAEDLGIYYCWQGTHFP
GTFGGGTKLEIK;
(SEQ ID NO: 26)
wherein CDR1 is KSSQSLLDSDGKTYLN,
(SEQ ID NO: 27)
CDR2 is LVSKLDS,
and
(SEQ ID NO: 28)
CDR3 is WQGTHFPGT.

The predicted CDR designations for the EGFRvIII CAR under Chothia are as follows:

VH:
(SEQ ID NO: 29);
EIQLQQSGAELVKPGASVKLSCTGSGFNIEDYYIHWVKQRTEQGLEWIGR
IDPENDETKYGPIFQGRATITADTSSNTVYLQLSSLTSEDTAVYYCAFRG
GVYWGPGTTLTVSS;
(SEQ ID NO: 30)
wherein CDR1 is GFNIEDY,
(SEQ ID NO: 31)
CDR2 is DPENDE,
and
(SEQ ID NO: 32)
CDR3 is RGGVY.
VL:
(SEQ ID NO: 33);
DVVMTQSPLTLSVAIGQSASISCKSSQSLLDSDGKTYLNWLLQRPGQSPK
RLISLVSKLDSGVPDRFTGSGSGTDFTLRISRVEAEDLGIYYCWQGTHFP
GTFGGGTKLEIK;
(SEQ ID NO: 34)
wherein CDR1 is SQSLLDSDGKTY,
(SEQ ID NO: 35)
CDR2 is LYS,
and
(SEQ ID NO: 36)
CDR3 is GTHFPG.

Humanization of murine EGFRvIII antibody is desired for the clinical setting, where the mouse-specific residues may induce a human-anti-mouse antigen (HAMA) response in patients who receive treatment with T cells transduced with the murine CAR construct. VH and VL sequences of hybridoma derived murine EGFRvIII antibody were extracted from published literature (Morgan et al. (2012) Human Gene Therapy, 23: 1043-1953, Supra). Humanization was accomplished by grafting CDR regions from murine EGFRvIII antibody onto human germline acceptor frameworks VH1_1-f or VH5_5a as well as VK2_A17 or VK4_B3 (vBASE database). In addition to the CDR regions, several framework residues, i.e. VK2 #36, #49, VK4 #2, #36, #46, #49, VH1 #2, #24, #76, #94 and VH5 #2, #24, #73, #76, #94, thought to support the structural integrity of the CDR regions were retained from the murine sequence. Further, the human J elements JH6 and JK4 were used for the heavy and light chain, respectively. The resulting amino acid sequences of the humanized antibody were designated VK2_A17/Hz1 and VK4_B3/Hz1 for the light-chains and VH1_1-f/Hz1, VH5_5-a/Hz1 for the heavy chains shown in FIG. 9 of US2014/0322275A1. The residue numbering follows Kabat (Kabat E. A. et al, 1991, supra). For CDR definitions, both Kabat as well as Chothia et al, 1987 supra) were used. Frame work residues retained from mouse EGFRvIII are shown boxed bold/italic, CDR residues are underlined.

Based on the humanized light and heavy chain sequences shown in FIG. 9 of US2014/0322275A1, a total of 8 framework combinations were used to generate soluble scFv's for further validation. The order in which the VL and VH domains appear in the scFv was varied (i.e., VL-VH, or VH-VL orientation), and four copies of the “G4S” subunit (SEQ ID NO: 37), in which each subunit comprises the sequence GGGGS (SEQ ID NO:37) was used to connect the frameworks. FIG. 9 of US2014/0322275A1discloses the CDR's in the VH and VL sequences calculated by Kabat et al and Chothia et al. (Supra).

### Cloning:

DNA sequences coding for mouse and humanized VL and VH domains were obtained, and the codons for the constructs were optimized for expression in cells from Homo sapiens.

Sequences coding for VL and VH domain were subcloned into expression vectors suitable for secretion in mammalian cells. Elements of the expression vector include a promoter (Cytomegalovirus (CMV) enhancer-promoter), a signal sequence to facilitate secretion, a polyadenylation signal and transcription terminator (Bovine Growth Hormone (BGH) gene), an element allowing episomal replication and replication in prokaryotes (e.g. SV40 origin and

ColE1 or others known in the art) and elements to allow selection (ampicillin resistance gene and zeocin marker).

Characterization of Humanized Anti-EGFRvIII Soluble scFv Fragments

Soluble scFv fragments were generated described above using standard molecule biology techniques. These soluble scFvs were used in characterization studies to examine the stability, cell surface expression, and binding properties of the scFvs.

scFv Expression and Purification

For transfection of each scFv construct, approximately 3e8 293F cells were transfected with 100m of plasmid using PEI as the transfection reagent at the ratio of 3:1 (PEI:DNA). The cells were grown in 100 ml EXPi293 Expression media (Invitrogen) in a shaker flask at 37° C., 125 rpm, 8% CO₂. The culture was harvested after six days and used for protein purification.

293F cells were harvested by spinning down at 3500 g for 20 minutes. The supernatant was collected and filtered through VacuCap90 PF Filter Unit (w/0.8/0.2 μm Super Membrane, PALL). Around 400 ul of Ni-NTA agarose beads (Qiagen) were added to the supernatant. The mixture was rotated and incubated for 4 hrs at 4° C. It was loaded onto a purification column and washed with washing buffer with 20 mM Histidine. The protein was eluted with 500p1 elution buffer with 300 mM Histidine. The samples were dialyzed against PBS buffer at 4C overnight. Protein samples were quantified using nanodrop 2000c.

EC₅₀ by FACS Binding of Purified scFv's to Cells Expressing Either Human EGFR Wild Type or EGFRvIII

The following experiments were conducted to demonstrate that all the humanized EGFRvIII scFv variants have comparable binding to EGFRvIII, but no binding to wild type EGFR.

HEK293F suspension cells were transiently transfected with either wild type hEGFR or hEGFRvIII and were harvested 2 days after transfection. Approximately 5e5 cells/per well were transferred to a BD Falcon 96 well plate. The cells were spun down at 900 rpm (Sorval Legend XT centrifuge) for 3 minutes. The supernatant was removed. Anti-EGFRvIII scFv protein samples were diluted in DPBS with 5% FBS. The samples were added into the wells, mixed and incubated for 1 hour. The cells were washed twice in the DPBS with 5% FBS. The cells were incubated with anti-poly His PE (R&D) for 1 hour, washed twice before FACS analysis (LSRII from BD Biosciences).

The EC₅₀ of mouse scFv (m3C10) for hEGFRvIII was determined to be ˜5 nM as shown in FIG. 10 of US2014/0322275A1. All the humanized EGFRvIII scFv variants showed EC₅₀ values in the single digit to low double digit nM EC₅₀s range (5-50 nM), Moreover, no appreciable binding of constructs 2173 and 2174 to wild type EGFR expressing cell lines was detected indicating an improved safety profile compared to murine 3C10, as shown in FIG. 11 of US2014/0322275A1.

### Humanized EGFRvIII CAR Constructs

ScFv to be used in the final CAR constructs were derived from the humanized framework sequences described in Example 1. The order in which the VL and VH domains appear in the scFv was varied (i.e., VL-VH, or VH-VL orientation). A (G45)₄ (SEQ ID NO: 113), linker was used to connect the variable domains to create the scFvs shown in Table 1.

TABLE 1
Humanized EGFRvIII scFv constructs showing
VH and VL orientation and linker length (Table
discloses “G4S” as SEQ ID NO: 37)

| construct ID | Length aa | annotation |
|---|---|---|
| 108358 | 277 | VH1-VK4, 4G4S (SEQ ID NO: 113) |
| 108359 | 277 | VK4-VH1, 4G4S (SEQ ID NO: 113) |
| 108360 | 277 | VH5-VK2, 4G4S (SEQ ID NO: 113) |
| 108361 | 277 | VK2-VH5, 4G4S (SEQ ID NO: 113) |
| 107276 | 277 | VH1-VK2, 4G4S (SEQ ID NO: 113) |
| 111046 | 278 | VH5-VK4, 4G4S (SEQ ID NO: 113) |
| 111048 | 278 | VK4-VH5, 4G4S (SEQ ID NO: 113) |
| 107277 | 277 | VK2-VH1, 4G4S (SEQ ID NO: 113) |
| 107275 | | |
| mEGFRvIII 3C10 | 274 | VH-VL, 3G4S (SEQ ID NO: 114) + HM |
| EGFRvIII 139 | 269 | VL-VH, |

The sequences of the humanized scFv fragments are provided below in Table 2 (SEQ ID NO:38, SEQ ID NO:44, SEQ ID NO:50, SEQ ID NO:56, SEQ ID NO:62, SEQ ID NO:68, SEQ ID NO:74, and SEQ ID NO:80). These scFv fragments were used with additional sequences, SEQ ID NOs: 13-17, to generate full CAR constructs with SEQ ID NOs: SEQ ID NO:43, SEQ ID NO:49, SEQ ID NO:55, SEQ ID NO:61, SEQ ID NO:67, SEQ ID NO:73, SEQ ID NO:79, and SEQ ID NO:85.

These clones all contained a Q/K residue change in the signal domain of the co-stimulatory domain derived from CD3zeta chain.

TABLE 2
Humanized EGFRvIII CAR Constructs

| | | |
|---|---|---|
| Name | SEQ ID NO: | Sequence |

CAR 1

| | | |
|---|---|---|
| CAR1 | 38 | eiqlvqsgeavkkpgatvkisckgsgfniedyyihwvqqapgkglewmgridpendet |
| scFv | | kygpifqgrvtitadtstntvymelsslrsedtayycafrggvywgqgttvtvssggg |
| domain | | gsggggsggggsggggsdvvmtqspdslavslgeratinckssqslldsdgktylnwl |
| | | qqkpgqppkrlislvskldsgvpdrfsgsgsgtdftltisslqaedvavyycwqgthf |
| | | pgtfgggtkveik |
| CAR1 | 39 | gaaatccagctggtccaatcgggagctgaggtcaagaagccgggagccaccgtcaaga |
| scFv | | tctcatgcaaggggtcgggattcaacatcgaggactactacattcactgggtgcagca |
| domain nt | | agctccgggaaaaggcctggaatggatgggcagaatcgacccagaaaacgacgaaact |
| | | aagtacggaccgattttccaaggaagagtgactatcaccgccgatacttcaaccaata |
| | | ccgtctacatggaactgagctcgctccggtccgaagatactgcagtgtattactgtgc |
| | | ctttcgcggaggggtgtactggggccaaggaactactgtcactgtctcgtcaggaggc |
| | | ggagggtcgggaggaggcgggagcggaggcggtggctcgggtggcggaggaagcgacg |
| | | tggtgatgacccagtccccggactccctcgccgtgagcctcggagagagggcgactat |
| | | caattgcaagtcgtcccagtcacttctggattccgatggtaaaacgtacctcaactgg |
| | | ctgcagcaaaagccagggcagccacccaaacggttgatctcccttgtgtccaaactgg |
| | | atagcggagtgcctgaccgcttctcgggttccggtagcgggaccgacttcaccctgac |
| | | gatcagctcactgcaggcggaggacgtggcagtgtactactgctggcagggaacccac |
| | | ttccctggcacctttggaggtggcaccaaggtggagatcaag |
| CAR1 | 40 | atggccctccctgtcaccgccctgctgcttccgctggctcttctgctccacgccgctc |
| Soluble | | ggcccgaaatccagctggtccaatcgggagctgaggtcaagaagccgggagccaccgt |
| scFv-nt | | caagatctcatgcaaggggtcgggattcaacatcgaggactactacattcactgggtg |
| | | cagcaagctccgggaaaaggcctggaatggatgggcagaatcgacccagaaaacgacg |
| | | aaactaagtacggaccgattttccaaggaagagtgactatcaccgccgatacttcaac |
| | | caataccgtctacatggaactgagctcgctccggtccgaagatactgcagtgtattac |
| | | tgtgcctttcgcggaggggtgtactggggccaaggaactactgtcactgtctcgtcag |
| | | gaggcggagggtcgggaggaggcgggagcggaggcggtggctcgggtggcggaggaag |
| | | cgacgtggtgatgacccagtccccggactccctcgccgtgagcctcggagagagggcg |
| | | actatcaattgcaagtcgtcccagtcacttctggattccgatggtaaaacgtacctca |
| | | actggctgcagcaaaagccagggcagccacccaaacggttgatctcccttgtgtccaa |
| | | actggatagcggagtgcctgaccgcttctcgggttccggtagcgggaccgacttcacc |
| | | ctgacgatcagctcactgcaggcggaggacgtggcagtgtactactgctggcagggaa |
| | | cccacttccctggcacctttggaggtggcaccaaggtggagatcaagggatcgcacca |
| | | ccatcaccatcatcatcac |
| CAR1 | 41 | Malpvtalllplalllhaarpqiqlvqsgaevkkpgatvkisckgsgfniedyyihwv |
| Soluble | | qqapgkglewmgridpendetkygpifqgrvtitadtstntvymelsslrsedtavyy |
| scFv-nt | | cafrggvywgqgttvtvssggggsggggsggggsggggsdvvmtqspdslavslgera |
| | | tinckssqslldsdgktylnwlqqkpgqppkrlislvskldsgvpdrfsgsgsgtdft |
| | | ltisslqaedvavyycwqgthfpgtfgggtkveikgshhhhhhhh |
| CAR 1- | 42 | atggccctccctgtcaccgccctgctgcttccgctggctcttctgctccacgccgctc |
| Full-nt | | ggcccgagatccagctggtgcagtcgggagctgaagtcaaaaagcctggcgcaaccgt |
| lentivirus | | caagatctcgtgcaaaggatcagggttcaacatcgaggactactacatccattgggtg |
| | | caacaggcacccggaaaaggcctggagtggatggggaggattgacccagaaaatgacg |
| | | aaaccaagtacggaccgatcttccaaggacgggtgaccatcacggctgacacttccac |
| | | taacaccgtctacatggaactctcgagccttcgctcggaagataccgcggtgtactac |
| | | tgcgcctttagaggtggagtctactggggacaagggactaccgtcaccgtgtcgtcag |
| | | gtggcggaggatcaggcggaggcggctccggtggaggaggaagcggaggaggtggctc |
| | | cgacgtggtgatgacgcagtcaccggactccttggcggtgagcctgggtgaacgcgcc |
| | | actatcaactgcaagagctcccagagcttgctggactccgatggaaagacttatctca |
| | | attggctgcaacagaagcctggccagccgccaaagagactcatctcactggtgagcaa |
| | | gctggatagcggagtgccagatcggttttcgggatcgggctcaggcaccgacttcacc |
| | | ctgactatttcctccctccaagccgaggatgtggccgtctactactgttggcagggga |
| | | ctcacttcccggggaccttcggtggaggcactaaggtggagatcaaaaccactacccc |
| | | agcaccgaggccacccaccccggctcctaccatcgcctcccagcctctgtccctgcgt |
| | | ccggaggcatgtagacccgcagctggtggggccgtgcatacccggggtcttgacttcg |
| | | cctgcgatatctacatttgggcccctctggctggtacttgcggggtcctgctgctttc |
| | | actcgtgatcactctttactgtaagcgcggtcggaagaagctgctgtacatctttaag |
| | | caacccttcatgaggcctgtgcagactactcaagaggaggacggctgttcatgccggt |
| | | tcccagaggaggaggaaggcggctgcgaactgcgcgtgaaattcagccgcagcgcaga |
| | | tgctccagcctacaagcaggggcagaaccagctctacaacgaactcaatcttggtcgg |
| | | agagaggagtacgacgtgctggacaagcggagaggacgggacccagaaatgggcggga |
| | | agccgcgcagaaagaatccccaagagggcctgtacaacgagctccaaaaggataagat |
| | | ggcagaagcctatagcgagattggtatgaaaggggaacgcagaagaggcaaaggccac |
| | | gacggactgtaccagggactcagcaccgccaccaaggacacctatgacgctcttcaca |
| | | tgcaggccctgccgcctcgg |
| CAR 1- | 43 | malpvtalllplalllhaarpeiqlvqsgaevkkpgatvkisckgsgfniedyyihwv |
| Full-aa | | qqapgkglewmgridpendetkygpifqgrvtitadtstntvymelsslrsedtavyy |
| | | cafrggvywgqgttvtvssggggsggggsggggsggggsdvvmtqspdslavslgera |
| | | tinckssqslldsdgktylnwlqqkpgqppkrlislvskldsgvpdrfsgsgsgtdft |
| | | ltisslqaedvavyycwqgthfpgtfgggtkveiktttpaprpptpaptiasqplslr |
| | | peacrpaaggavhtrgldfacdiyiwaplagtcgvlllslvitlyckrgrkkllyifk |
| | | qpfmrpvqttqeedgcscrfpeeeeggcelrvkfsrsadapaykqgqnqlynelnlgr |
| | | reeydvldkrrgrdpemggkprrknpqeglynelqkdkmaeayseigmkgerrrgkgh |
| | | dglyqglstatkdtydalhmqalppr |

CAR 2

| | | |
|---|---|---|
| CAR2 | 44 | dvvmtqspdslavslgeratinckssqslldsdgktylnwlqqkpgqppkrlislvsk |
| scFv | | ldsgvpdrfsgsgsgtdftltisslqaedvavyycwqgthfpgtfgggtkveikgggg |
| domain | | sggggsggggsggggseiqlvqsgaevkkpgatvkisckgsgfniedyyihwvqqapg |
| | | kglewmgridpendetkygpifqgrvtitadtstntvymelsslrsedtavyycafrg |
| | | gvywgqgttvtvss |
| CAR2 | 45 | gatgtcgtgatgacccagtccccagactccctcgcagtgtccttgggagaacgggcca |
| scFv | | ccatcaactgcaaatcgagccagtcactgctggactcagacggaaagacctacctcaa |
| domain-nt | | ctggctgcagcagaagcctggccagccaccgaagcgcctgatctccctggtgtccaag |
| | | ctggactcgggcgtcccggacaggtttagcggtagcggctcgggaaccgacttcactc |
| | | tgaccattagctcgctccaagctgaagatgtggcggtctactactgctggcaggggac |
| | | ccacttccccgggacctttggcggaggaactaaagtcgaaatcaaaggaggaggcgga |
| | | tcaggtggaggaggcagcggaggaggagggagcggcggtggcggctccgaaattcaac |
| | | ttgtgcaatccggtgccgaggtgaagaaacctggtgccactgtcaagatctcgtgtaa |
| | | gggatcgggattcaatatcgaggactactacatccactgggtgcaacaggcgccagga |
| | | aagggattggagtggatgggtcgcatcgacccggaaaacgatgagactaagtacggac |
| | | cgatcttccaaggccgggtcacgatcactgcggatacctccactaataccgtgtatat |
| | | ggagctctcgtcactgagaagcgaagatacggccgtgtactactgcgcattcagagga |
| | | ggtgtgtactggggccagggaactactgtgaccgtgtcgtcg |
| CAR2- | 46 | atggccctccctgtcaccgccctgctgcttccgctggctcttctgctccacgccgctc |
| Soluble | | ggcccgatgtcgtgatgacccagtccccagactccctcgcagtgtccttgggagaacg |
| scFv-nt | | ggccaccatcaactgcaaatcgagccagtcactgctggactcagacggaaagacctac |
| | | ctcaactggctgcagcagaagcctggccagccaccgaagcgcctgatctccctggtgt |
| | | ccaagctggactcgggcgtcccggacaggtttagcggtagcggctcgggaaccgactt |
| | | cactctgaccattagctcgctccaagctgaagatgtggcggtctactactgctggcag |
| | | gggacccacttccccgggacctttggcggaggaactaaagtcgaaatcaaaggaggag |
| | | gcggatcaggtggaggaggcagcggaggaggagggagcggcggtggcggctccgaaat |
| | | tcaacttgtgcaatccggtgccgaggtgaagaaacctggtgccactgtcaagatctcg |
| | | tgtaagggatcgggattcaatatcgaggactactacatccactgggtgcaacaggcgc |
| | | caggaaagggattggagtggatgggtcgcatcgacccggaaaacgatgagactaagta |
| | | cggaccgatcttccaaggccgggtcacgatcactgcggatacctccactaataccgtg |
| | | tatatggagctctcgtcactgagaagcgaagatacggccgtgtactactgcgcattca |
| | | gaggaggtgtgtactggggccagggaactactgtgaccgtgtcgtcggggtcacatca |
| | | ccaccatcatcatcaccac |
| CAR2- | 47 | malpvtalllplalllhaarpdvvmtqspdslavslgeratinckssqslldsdgkty |
| Soluble | | lnwlqqkpgqppkrlislvskldsgvdprfsgsgsgtdftltisslqaedvavyycwq |
| scFv-nt | | gthfpgtfgggtkveikggggsggggsggggsggggseiqlvqsgaevkkpgatvkis |
| | | ckgsgfniedyyihwvqqapgkglewmgridpendetkygpifqgrvtitadtstntv |
| | | ymelsslrsedtavyycafrggvywgqgttvtvssgshhhhhhhh |
| CAR2- | 48 | atggccctccctgtcaccgccctgctgcttccgctggctcttctgctccacgccgctc |
| Full-nt | | ggcccgacgtggtcatgactcaaagcccagattccttggctgtctcccttggagaaag |
| | | agcaacgatcaattgcaaaagctcgcagtccctgttggactccgatggaaaaacctac |
| | | ctcaactggctgcagcagaagccgggacaaccaccaaagcggctgatttccctcgtgt |
| | | ccaagctggacagcggcgtgccggatcgcttctcgggcagcggctcgggaaccgattt |
| | | tactctcactatttcgtcactgcaagcggaggacgtggcggtgtattactgctggcag |
| | | ggcactcacttcccgggtacttttggtggaggtaccaaagtcgaaatcaagggtggag |
| | | gcgggagcggaggaggcgggtcgggaggaggaggatcgggtggcggaggctcagaaat |
| | | ccagctggtgcagtcaggtgccgaagtgaagaagcctggggccacggtgaagatctcg |
| | | tgcaaggggagcggattcaacatcgaggattactacatccattgggtgcaacaggccc |
| | | ctggcaaagggctggaatggatgggaaggatcgaccccgagaatgacgagactaagta |
| | | cggcccgatcttccaaggacgggtgaccatcactgcagacacttcaaccaacaccgtc |
| | | tacatggaactctcctcgctgcgctccgaggacaccgccgtgtactactgtgctttca |
| | | gaggaggagtctactggggacagggaacgaccgtgaccgtcagctcaaccactacccc |
| | | agcaccgaggccacccaccccggctcctaccatcgcctcccagcctctgtccctgcgt |
| | | ccggaggcatgtagacccgcagctggtggggccgtgcatacccggggtcttgacttcg |
| | | cctgcgatatctacatttgggcccctctggctggtacttgcggggtcctgctgctttc |
| | | actcgtgatcactctttactgtaagcgcggtcggaagaagctgctgtacatctttaag |
| | | caacccttcatgaggcctgtgcagactactcaagaggaggacggctgttcatgccggt |
| | | tcccagaggaggaggaaggcggctgcgaactgcgcgtgaaattcagccgcagcgcaga |
| | | tgctccagcctacaagcaggggcagaaccagctctacaacgaactcaatcttggtcgg |
| | | agagaggagtacgacgtgctggacaagcggagaggacgggacccagaaatgggcggga |
| | | agccgcgcagaaagaatccccaagagggcctgtacaacgagctccaaaaggataagat |
| | | ggcagaagcctatagcgagattggtatgaaaggggaacgcagaagaggcaaaggccac |
| | | gacggactgtaccagggactcagcaccgccaccaaggacacctatgacgctcttcaca |
| | | tgcaggccctgccgcctcgg |
| CAR 2- | 49 | malpvtalllplalllhaarpdvvmtqspdslavslgeratinckssqslldsdgkty |
| Full-aa | | lnwlqqkpgqppkrlislvskldsgvpdrfsgsgsgtdftltisslqaedvavyycwq |
| | | gthfpgtfgggtkveikggggsggggsggggsggggseqilvqsgaevkkpgatvkis |
| | | ckgsgfniedyyihwvqqapgkglewmgridpendetkygpirqgrvtitadtstntv |
| | | ymelsslrsedtavyycafrggvywgqgttvtvsstttpaprpptpaptiasqplslr |
| | | peacrpaaggavhtrgldfacdiyiwaplagtcgvlllslvitlyckrgrkkllyifk |
| | | qpfmrpvqqtqeedgcscrfpeeeeggcelrvkfsrsadapaykqgqnqlynelnlgr |
| | | reeydvldkrrgrdpemggkprrknpqeglynelqkdkmaeayseigmkgerrrgkgh |
| | | dglyqglstatkdtydalhmqalppr |

CAR 3

| | | |
|---|---|---|
| CAR3 | 50 | eiqlvqsgaevkkpgeslrisckgsgfniedyyihwvrqmpgklgewmgridpendet |
| scFv | | kygpifqghvtisadtsintvylqwsslkasdtamyycafrggvywgqgttvtvssgg |
| domain | | ggsggggsggggsggggsdvvmtqsplslpvtlgqpasisckssqslldsdgktylnw |
| | | lqqrpgqsprrlislvskldsgvpdrfsgsgsgtdftlkisrveaedvgvyycwqgth |
| | | fpgtfgggtkveik |
| CAR3 | 51 | gagattcagctggtccaaagcggcgcagaagtgaaaaagccaggggaatcgttgcgca |
| scFv | | tcagctgtaaaggttccggcttcaacatcgaggactattacatccattgggtgcggca |
| domain nt | | gatgccaggaaaggggctggaatggatgggacggattgacccggagaacgacgaaacc |
| | | aagtacggaccgatctttcaaggacacgtgactatctccgccgacaccagcatcaata |
| | | cggtgtacctccaatggtcctcactcaaggcctcggataccgcgatgtactactgcgc |
| | | gttcagaggaggcgtctactggggacaagggactactgtgactgtctcatcaggaggt |
| | | ggaggaagcggaggaggtggctcgggcggaggtggatcgggaggaggagggtccgatg |
| | | tggtgatgacccagtccccactgtcgctcccggtgaccctcggacagcctgctagcat |
| | | ctcgtgcaaatcctcgcaatccctgctggactcggacggaaaaacgtacctcaattgg |
| | | ctgcagcagcgccctggccagagcccgagaaggcttatctcgctggtgtcaaagctgg |
| | | atagcggtgtgcccgaccggttcagcggctcagggtcaggaaccgatttcaccttgaa |
| | | gatctcccgcgtggaagccgaagatgtcggagtctactactgctggcagggtactcac |
| | | ttcccggggacctttggtggcggcactaaggtcgagattaag |
| CAR 3- | 52 | atggccctccctgtcaccgccctgctgcttccgctggctcttctgctccacgccgctc |
| Soluble | | ggcccgagattcagctggtccaaagcggcgcagaagtgaaaaagccaggggaatcgtt |
| scFv-nt | | gcgcatcagctgtaaaggttccggcttcaacatcgaggactattacatccattgggtg |
| | | cggcagatgccaggaaaggggctggaatggatgggacggattgacccggagaacgacg |
| | | aaaccaagtacggaccgatctttcaaggacacgtgactatctccgccgacaccagcat |
| | | caatacggtgtacctccaatggtcctcactcaaggcctcggataccgcgatgtactac |
| | | tgcgcgttcagaggaggcgtctactggggacaagggactactgtgactgtctcatcag |
| | | gaggtggaggaagcggaggaggtggctcgggcggaggtggatcgggaggaggagggtc |
| | | cgatgtggtgatgacccagtccccactgtcgctcccggtgaccctcggacagcctgct |
| | | agcatctcgtgcaaatcctcgcaatccctgctggactcggacggaaaaacgtacctca |
| | | attggctgcagcagcgccctggccagagcccgagaaggcttatctcgctggtgtcaaa |
| | | gctggatagcggtgtgcccgaccggttcagcggctcagggtcaggaaccgatttcacc |
| | | ttgaagatctcccgcgtggaagccgaagatgtcggagtctactactgctggcagggta |
| | | ctcacttcccggggacctttggtggcggcactaaggtcgagattaagggctcacacca |
| | | tcatcaccatcaccaccac |
| CAR 3- | 53 | malpvtalllplalllhaarpeiqlvqsgaevkkpgeslrisckgsgfniedyyihwv |
| Soluble | | rqmpgkglewmgridpendetkygpifqghvtisadtsintvylqwsslkasdtamyy |
| scFv-aa | | cafrggvywgqgttvtvssggggsggggsggggsggggsdvvmtqsplslpvtlgqpa |
| | | sisckssqslldsdgktylnwlqqrpgqsprrlislvskldsgvpdrfsgsgsgtdft |
| | | lkisrveaedvtvyycwqgthfpgtfgggtkveikgshhhhhhhh |
| CAR 3- | 54 | atggccctccctgtcaccgccctgctgcttccgctggctcttctgctccacgccgctc |
| Full-nt | | ggcccgaaatccagctggtgcaaagcggagccgaggtgaagaagcccggagaatccct |
| | | gcgcatctcgtgtaagggttccggctttaacatcgaggattactacatccactgggtg |
| | | agacagatgccgggcaaaggtctggaatggatgggccgcatcgacccggagaacgacg |
| | | aaaccaaatacggaccaatcttccaaggacatgtgactatttccgcggatacctccat |
| | | caacactgtctacttgcagtggagctcgctcaaggcgtcggataccgccatgtactac |
| | | tgcgcattcagaggaggtgtgtactggggccagggcactacggtcaccgtgtcctcgg |
| | | gaggtggagggtcaggaggcggaggctcgggcggtggaggatcaggcggaggaggaag |
| | | cgatgtggtcatgactcaatccccactgtcactgcctgtcactctggggcaaccggct |
| | | tccatctcatgcaagtcaagccaatcgctgctcgactccgacggaaaaacctacctca |
| | | attggcttcagcagcgcccaggccagtcgcctcggaggctgatctcactcgtgtcgaa |
| | | gcttgactccggggtgccggatcggtttagcggaagcggatcggggaccgacttcacg |
| | | ttgaagattagccgggtggaagccgaggacgtgggagtctattactgctggcagggga |
| | | cccacttcccggggactttcggaggaggcaccaaagtcgagattaagaccactacccc |
| | | agcaccgaggccacccaccccggctcctaccatcgcctcccagcctctgtccctgcgt |
| | | ccggaggcatgtagacccgcagctggtggggccgtgcatacccggggtcttgacttcg |
| | | cctgcgatatctacatttgggcccctctggctggtacttgcggggtcctgctgctttc |
| | | actcgtgatcactctttactgtaagcgcggtcggaagaagctgctgtacatctttaag |
| | | caacccttcatgaggcctgtgcagactactcaagaggaggacggctgttcatgccggt |
| | | tcccagaggaggaggaaggcggctgcgaactgcgcgtgaaattcagccgcagcgcaga |
| | | tgctccagcctacaagcaggggcagaaccagctctacaacgaactcaatcttggtcgg |
| | | agagaggagtacgacgtgctggacaagcggagaggacgggacccagaaatgggcggga |
| | | agccgcgcagaaagaatccccaagagggcctgtacaacgagctccaaaaggataagat |
| | | ggcagaagcctatagcgagattggtatgaaaggggaacgcagaagaggcaaaggccac |
| | | gacggactgtaccagggactcagcaccgccaccaaggacacctatgacgctcttcaca |
| | | tgcaggccctgccgcctcgg |
| CAR 3- | 55 | malpvtalllplalllhaarpeiqlvqsgaevkkpgeslrisckgsgfniedyyihwv |
| Full-aa | | rqmpgkglewmgridpendetkygpifqghvtisadtsintvylqwsslkasdtamyy |
| | | cafrggvywgqgttvtvssggggsggggsggggsggggsdvvmtqsplslpvtlgpas |
| | | isckssqslldsdgktylnwlqqrpgqsprrlislvskldsgvpdrfsgsgsgtdftl |
| | | kisrveadvgvyycwqgthfpgtfgggtkveiktttpaprpptpaptiasqplslrpe |
| | | acrpaaggavhtrgldfacdiyiwaplagtcgvlllslvitlyckrgrkkllyifkqp |
| | | fmrpvqqtqeedgcscrfpeeeeggcelrvkfsrsadapaykqgqnqlynelnlgrre |
| | | eydvldkrrgrdpemggkprrknpqeglynelqkdkmaeayseigmkgerrrgkghdg |
| | | lyqglstatkdtydalhmqalppr |

CAR 4

| | | |
|---|---|---|
| CAR4 | 56 | dvvmtqsplslpvtlgqpasisckssqslldsdgktylnwlqqrpgqsprrlislvsk |
| scFv | | ldsgvpdrfsgsgsgtdftlkisrveaedvgvyycwqgthfpgtfgggtkveikgggg |
| domain | | sggggsggggsggggseiqlvqsgaevkkpgeslrisckgsgfniedyyihwvrqmpg |
| | | kglewmgridpendetkygpifqghvtisadtsintvylqwsslkasdtamyycafrg |
| | | gvywgqgttvtvss |
| CAR4 | 57 | gacgtcgtcatgacccagagcccgctgtcactgcctgtgaccctgggccagccggcgt |
| scFv | | ccattagctgcaaatcctcgcaatccctgctcgactcagacggaaaaacgtacttgaa |
| domain nt | | ctggctccaacagcgccctgggcaatccccaaggcggcttatctcactcgtcagcaag |
| | | ctcgatagcggtgtcccagacagattttcgggctcgggatcgggcactgatttcactc |
| | | tgaagatctcgcgggtggaagccgaggatgtgggagtgtactattgctggcagggcac |
| | | tcacttccccgggacgtttggcggaggaactaaggtcgagatcaaaggaggaggtgga |
| | | tcaggcggaggtgggagcggaggaggaggaagcggtggtggaggttccgaaatccagc |
| | | tggtgcaatcaggagccgaggtgaagaagccgggagaatccctgcgcatctcgtgcaa |
| | | gggctcgggcttcaacatcgaggattactacatccactgggtgcggcagatgccggga |
| | | aaggggttggaatggatgggacgcattgacccggaaaatgatgaaaccaaatacgggc |
| | | caatcttccaaggccacgtgaccattagcgctgacacttccatcaacaccgtgtacct |
| | | tcagtggtcctcactgaaggcgtcggacactgccatgtactactgtgcattcagagga |
| | | ggggtctactggggacagggcaccaccgtgaccgtgagctcc |
| CAR4- | 58 | atggccctccctgtcaccgccctgctgcttccgctggctcttctgctccacgccgctc |
| Soluble | | ggcccgacgtcgtcatgacccagagcccgctgtcactgcctgtgaccctgggccagcc |
| scFv-nt | | ggcgtccattagctgcaaatcctcgcaatccctgctcgactcagacggaaaaacgtac |
| | | ttgaactggctccaacagcgccctgggcaatccccaaggcggcttatctcactcgtca |
| | | gcaagctcgatagcggtgtcccagacagattttcgggctcgggatcgggcactgattt |
| | | cactctgaagatctcgcgggtggaagccgaggatgtgggagtgtactattgctggcag |
| | | ggcactcacttccccgggacgtttggcggaggaactaaggtcgagatcaaaggaggag |
| | | gtggatcaggcggaggtgggagcggaggaggaggaagcggtggtggaggttccgaaat |
| | | ccagctggtgcaatcaggagccgaggtgaagaagccgggagaatccctgcgcatctcg |
| | | tgcaagggctcgggcttcaacatcgaggattactacatccactgggtgcggcagatgc |
| | | cgggaaaggggttggaatggatgggacgcattgacccggaaaatgatgaaaccaaata |
| | | cgggccaatcttccaaggccacgtgaccattagcgctgacacttccatcaacaccgtg |
| | | taccttcagtggtcctcactgaaggcgtcggacactgccatgtactactgtgcattca |
| | | gaggaggggtctactggggacagggcaccaccgtgaccgtgagctccggctcgcatca |
| | | ccatcatcaccaccatcac |
| CAR4- | 59 | malpvtalllplalllhaardpvvmtqsplslpvtlgqpasisckssqslldsdgkty |
| Soluble | | lnwlqqrpgqsprrlislvskldsgvpdrfsgsgsgtdftlkisrveadedvgvyycw |
| scFv-aa | | qgtfpgtfgggtkveikggggsggggsggggsggggseiqlvqsgaevkkpgeslris |
| | | ckgsgfniedyyihwvrqmpgkglewmgridpendetkygpifqghvtisadtsintv |
| | | ylqwsslkasdtamyycafrggvywgqgttvtvssgshhhhhhhh |
| CAR 4- | 60 | atggccctccctgtcaccgccctgctgcttccgctggctcttctgctccacgccgctc |
| Full-nt | | ggcccgacgtcgtcatgacccaatcccctctctccctgccggtcaccctgggtcagcc |
| | | ggcgtcgatctcatgcaaaagctcacagtccctgctggattcggacggaaaaacctac |
| | | ttgaactggctccaacagaggccgggtcagtcccctcgcagactgatctcgctggtga |
| | | gcaagctcgactcgggtgtgccggatcggttctccgggtcaggatcgggcaccgactt |
| | | tacgctcaagatttcgagagtggaggccgaggatgtgggagtgtactattgctggcag |
| | | ggcacgcatttccccgggacctttggaggcgggactaaggtggaaatcaagggaggtg |
| | | gcggatcaggcggaggaggcagcggcggaggtggatcaggaggcggagggtcagagat |
| | | ccagctggtccaaagcggagcagaggtgaagaagccaggcgagtcccttcgcatttcg |
| | | tgcaaagggagcggcttcaacattgaagattactacatccactgggtgcggcaaatgc |
| | | caggaaagggtctggaatggatgggacggatcgacccagaaaatgatgaaactaagta |
| | | cggaccgatcttccaaggacacgtcactatctccgcggacacttcgatcaacaccgtg |
| | | tacctccagtggagcagcttgaaagcctccgacaccgctatgtactactgtgccttcc |
| | | gcggaggagtctactggggacaggggactactgtgaccgtgtcgtccaccactacccc |
| | | agcaccgaggccacccaccccggctcctaccatcgcctcccagcctctgtccctgcgt |
| | | ccggaggcatgtagacccgcagctggtggggccgtgcatacccggggtcttgacttcg |
| | | cctgcgatatctacatttgggcccctctggctggtacttgcggggtcctgctgctttc |
| | | actcgtgatcactctttactgtaagcgcggtcggaagaagctgctgtacatctttaag |
| | | caacccttcatgaggcctgtgcagactactcaagaggaggacggctgttcatgccggt |
| | | tcccagaggaggaggaaggcggctgcgaactgcgcgtgaaattcagccgcagcgcaga |
| | | tgctccagcctacaagcaggggcagaaccagctctacaacgaactcaatcttggtcgg |
| | | agagaggagtacgacgtgctggacaagcggagaggacgggacccagaaatgggcggga |
| | | agccgcgcagaaagaatccccaagagggcctgtacaacgagctccaaaaggataagat |
| | | ggcagaagcctatagcgagattggtatgaaaggggaacgcagaagaggcaaaggccac |
| | | gacggactgtaccagggactcagcaccgccaccaaggacacctatgacgctcttcaca |
| | | tgcaggccctgccgcctcgg |
| CAR 4- | 61 | malpvtalllplalllhaarpdvvmtqsplslpvtlgqpasisckssqslldsdgkty |
| Full-aa | | lnwlqqrpgqsprrlislvskldsgvpdrfsgsgsgtdftlkisrveaedvgvyycwq |
| | | gthfpgtfgggtkveikggggsggggsggggsggggseiqlvqsgaevkkpgeslris |
| | | ckgsgfniedyyihwvrqmpgklgewmgridpendetkvgpifqghvtisadtsintv |
| | | ylqwsslkasdtamyycafrggvvwgqgttvtvsstttpaprpptpaptiasqplslr |
| | | peacrpaaggavhtrgldfacdiyiwaplagtcgvlllslvitlyckrgrkkllyifk |
| | | qpfmrpvqttqeedgcscrfpeeeeggcelrvkfsrsadapaykqgqnqlynelnlgr |
| | | reeydvldkrrgrdpemggkprrknpqeglynelqkdkmaeayseigmkgerrrgkgh |
| | | dglyqglstatdtydalhmqalppr |

CAR 5

| | | |
|---|---|---|
| CAR5 | 62 | eiqlvqsgaevkkpgatvkisckgsgfniedyyihwvqqapgkglewmgridpendet |
| scFv | | kygpifqgrvtitadtstntvymelsslrsedtavyycafrggvywgqgttvtvssgg |
| domain | | ggsggggsggggsggggsdvvmtqsplslpvtlgqpasisckssqslldsdgktylnw |
| | | lqqrpgqsprrlislvskldsgvpdrfsgsgsgtdftlkisrveaedvgvyycwqgth |
| | | fpgtfgggtkveik |
| CAR5 | 63 | gaaatccagctcgtgcagagcggagccgaggtcaagaaaccgggtgctaccgtgaaga |
| scFv | | tttcatgcaagggatcgggcttcaacatcgaggattactacatccactgggtgcagca |
| domain nt | | ggcaccaggaaaaggacttgaatggatgggccggatcgacccggaaaatgacgagact |
| | | aagtacggccctatcttccaaggacgggtgacgatcaccgcagacactagcaccaaca |
| | | ccgtctatatggaactctcgtccctgaggtccgaagatactgccgtgtactactgtgc |
| | | gtttcgcggaggtgtgtactggggacagggtaccaccgtcaccgtgtcatcgggcggt |
| | | ggaggctccggtggaggagggtcaggaggcggtggaagcggaggaggcggcagcgacg |
| | | tggtcatgactcaatcgccgctgtcgctgcccgtcactctgggacaacccgcgtccat |
| | | cagctgcaaatcctcgcagtcactgcttgactccgatggaaagacctacctcaactgg |
| | | ctgcagcaacgcccaggccaatccccaagacgcctgatctcgttggtgtcaaagctgg |
| | | actcaggggtgccggaccggttctccgggagcgggtcgggcacggatttcactctcaa |
| | | gatctccagagtggaagccgaggatgtgggagtctactactgctggcagggaacccat |
| | | ttccctggaacttttggcggaggaactaaggtcgagattaaa |
| CAR5- | 64 | atggccctccctgtcaccgccctgctgcttccgctggctcttctgctccacgccgctc |
| Soluble | | ggcccgaaatccagctcgtgcagagcggagccgaggtcaagaaaccgggtgctaccgt |
| scFv-nt | | gaagatttcatgcaagggatcgggcttcaacatcgaggattactacatccactgggtg |
| | | cagcaggcaccaggaaaaggacttgaatggatgggccggatcgacccggaaaatgacg |
| | | agactaagtacggccctatcttccaaggacgggtgacgatcaccgcagacactagcac |
| | | caacaccgtctatatggaactctcgtccctgaggtccgaagatactgccgtgtactac |
| | | tgtgcgtttcgcggaggtgtgtactggggacagggtaccaccgtcaccgtgtcatcgg |
| | | gcggtggaggctccggtggaggagggtcaggaggcggtggaagcggaggaggcggcag |
| | | cgacgtggtcatgactcaatcgccgctgtcgctgcccgtcactctgggacaacccgcg |
| | | tccatcagctgcaaatcctcgcagtcactgcttgactccgatggaaagacctacctca |
| | | actggctgcagcaacgcccaggccaatccccaagacgcctgatctcgttggtgtcaaa |
| | | gctggactcaggggtgccggaccggttctccgggagcgggtcgggcacggatttcact |
| | | ctcaagatctccagagtggaagccgaggatgtgggagtctactactgctggcagggaa |
| | | cccatttccctggaacttttggcggaggaactaaggtcgagattaaagggagccacca |
| | | tcatcatcaccaccaccac |
| CAR5- | 65 | malpvtalllplalllhaarpeiqlvqsgaevkkpgatvkisckgsgfniedyyihwv |
| Soluble | | qqapgkglewmgridpendetkygpifqgrvtitadtstntvymelsslrsedtavyy |
| scFv-aa | | cafrggvywgqgttvtvssggggsggggsggggsggggsdvvmtqsplslpvtlgqpa |
| | | sisckssqslldsdgktylnwlqqrpgqsprrlislvkskldsgvpdrfsgsgsgtdf |
| | | tlkisrveaedvgvyycwqgthfp[gtfgggtkveikgshhhhhhhh |
| CAR 5- | 66 | atggccctccctgtcaccgccctgctgcttccgctggctcttctgctccacgccgctc |
| Full-nt | | ggcccgaaatccagctcgtgcagagcggagccgaggtcaagaaaccgggtgctaccgt |
| | | gaagatttcatgcaagggatcgggcttcaacatcgaggattactacatccactgggtg |
| | | cagcaggcaccaggaaaaggacttgaatggatgggccggatcgacccggaaaatgacg |
| | | agactaagtacggccctatcttccaaggacgggtgacgatcaccgcagacactagcac |
| | | caacaccgtctatatggaactctcgtccctgaggtccgaagatactgccgtgtactac |
| | | tgtgcgtttcgcggaggtgtgtactggggacagggtaccaccgtcaccgtgtcatcgg |
| | | gcggtggaggctccggtggaggagggtcaggaggcggtggaagcggaggaggcggcag |
| | | cgacgtggtcatgactcaatcgccgctgtcgctgcccgtcactctgggacaacccgcg |
| | | tccatcagctgcaaatcctcgcagtcactgcttgactccgatggaaagacctacctca |
| | | actggctgcagcaacgcccaggccaatccccaagacgcctgatctcgttggtgtcaaa |
| | | gctggactcaggggtgccggaccggttctccgggagcgggtcgggcacggatttcact |
| | | ctcaagatctccagagtggaagccgaggatgtgggagtctactactgctggcagggaa |
| | | cccatttccctggaacttttggcggaggaactaaggtcgagattaaaaccactacccc |
| | | agcaccgaggccacccaccccggctcctaccatcgcctcccagcctctgtccctgcgt |
| | | ccggaggcatgtagacccgcagctggtggggccgtgcatacccggggtcttgacttcg |
| | | cctgcgatatctacatttgggcccctctggctggtacttgcggggtcctgctgctttc |
| | | actcgtgatcactctttactgtaagcgcggtcggaagaagctgctgtacatctttaag |
| | | caacccttcatgaggcctgtgcagactactcaagaggaggacggctgttcatgccggt |
| | | tcccagaggaggaggaaggcggctgcgaactgcgcgtgaaattcagccgcagcgcaga |
| | | tgctccagcctacaagcaggggcagaaccagctctacaacgaactcaatcttggtcgg |
| | | agagaggagtacgacgtgctggacaagcggagaggacgggacccagaaatgggcggga |
| | | agccgcgcagaaagaatccccaagagggcctgtacaacgagctccaaaaggataagat |
| | | ggcagaagcctatagcgagattggtatgaaaggggaacgcagaagaggcaaaggccac |
| | | gacggactgtaccagggactcagcaccgccaccaaggacacctatgacgctcttcaca |
| | | tgcaggccctgccgcctcgg |
| CAR 5- | 67 | malpvtalllplalllhaarpeiqlvqsgacvkkpgatvkisckgsgfniedyyihwv |
| Full-aa | | qqapgkglewmgridpendetkygpifqgrvtitadtstntvymelsslrsedtavyy |
| | | cafrggvywgqgttvtvssggggsggggsggggsggggsdvvmtqsplslpvtlgqpa |
| | | sisckssqslldsdgkltylnwlqqrpgqsprrlislvskldsgvpdrfsgsgsgtdf |
| | | tlkisrveaedvgvyycwqgthfpgtfgggtkveiktttpaprpptpaptiasqplsl |
| | | rpeacrpaaggavhtrgldfacdiyiwaplagtcgvlllslvitlyckrgrkkllyif |
| | | kqpfmrpvqttqeedgcscrfpeeeeggcelrvkfsrsadapaykqgqnqlynelnlg |
| | | rreeydvldkrrgrdpemggkprrknpqeglynelqkdkmacayseigmkgerrrgkg |
| | | hdglyqglstatkdtydalhmqalppr |

CAR 6

| | | |
|---|---|---|
| CAR6 | 68 | eiqlvqsgaevkkpgeslrisckgsgfniedyyihwvrqmpgkglewmgridpendet |
| scFv | | kygpifqghvtisadtsintvylqwsslkasdtamyycafrggvywgqgttvtvssgg |
| domain | | ggsggggsggggsggggsdvvmtqspdslavslgeratinckssqslldsdgktylnw |
| | | lqqkpgqppkrlislvskldsgvpdrfsgsgsgtdftltisslqaedvavyycwqgth |
| | | fpgtfgggtkveik |
| CAR6 | 69 | gaaatccagctggtgcagtcaggcgccgaggtcaagaagccgggagagtcgctgagaa |
| scFv | | tctcgtgcaagggctcggggttcaacatcgaggactactacattcactgggtcaggca |
| domain nt | | gatgccgggaaagggactggaatggatgggccggatcgacccagaaaatgacgaaacc |
| | | aaatacgggccgatttttcaaggccacgtgactatcagcgcagacacgagcatcaaca |
| | | ctgtctacctccagtggtcctcgcttaaggccagcgataccgctatgtactactgcgc |
| | | attcagaggcggggtgtactggggacaaggaaccactgtgaccgtgagcagcggaggt |
| | | ggcggctcgggaggaggtgggagcggaggaggaggttccggcggtggaggatcagatg |
| | | tcgtgatgacccagtccccggactccctcgctgtctcactgggcgagcgcgcgaccat |
| | | caactgcaaatcgagccagtcgctgttggactccgatggaaagacttatctgaattgg |
| | | ctgcaacagaaaccaggacaacctcccaagcggctcatctcgcttgtgtcaaaactcg |
| | | attcgggagtgccagaccgcttctcggggtccgggagcggaactgactttactttgac |
| | | catttcctcactgcaagcggaggatgtggccgtgtattactgttggcagggcacgcat |
| | | ttccctggaaccttcggtggcggaactaaggtggaaatcaag |
| CAR6- | 70 | atggccctccctgtcaccgccctgctgcttccgctggctcttctgctccacgccgctc |
| Soluble | | ggcccgaaatccagctggtgcagtcaggcgccgaggtcaagaagccgggagagtcgct |
| scFv-nt | | gagaatctcgtgcaagggctcggggttcaacatcgaggactactacattcactgggtc |
| | | aggcagatgccgggaaagggactggaatggatgggccggatcgacccagaaaatgacg |
| | | aaaccaaatacgggccgatttttcaaggccacgtgactatcagcgcagacacgagcat |
| | | caacactgtctacctccagtggtcctcgcttaaggccagcgataccgctatgtactac |
| | | tgcgcattcagaggcggggtgtactggggacaaggaaccactgtgaccgtgagcagcg |
| | | gaggtggcggctcgggaggaggtgggagcggaggaggaggaccggcggtggaggatca |
| | | gatgtcgtgatgacccagtccccggactccctcgctgtctcactgggcgagcgcgcga |
| | | ccatcaactgcaaatcgagccagtcgctgaggactccgatggaaagacttatctgaat |
| | | tggctgcaacagaaaccaggacaacctcccaagcggctcatctcgcagtgtcaaaact |
| | | cgattcgggagtgccagaccgcactcggggtccgggagcggaactgacatactagacc |
| | | atacctcactgcaagcggaggatgtggccgtgtattactgaggcagggcacgcatacc |
| | | ctggaaccacggtggcggaactaaggtggaaatcaagggatcacaccaccatcatcac |
| | | catcaccaccat |
| CAR6- | 71 | malpvtalllplalllhaarpeiqlvqsgaevkkpgeslrisckgsgfniedyyihwv |
| Soluble | | rqmpgkglewmgridpendetkygpifqghvtisadtsintvylqwsslkasdtamyy |
| scFv-aa | | cafrggvywgqgttvtvssggggsggggsggggsggggsdvvmtqspdslavslgera |
| | | tinckssqslldsdgktylnwlqqkpgqppkrlislvskldsgvpdrfsgsgsgtdft |
| | | lisslqaedvavyycwqgthfpgtfgggtkveikgshhhhhhhh |
| CAR6- | 72 | atggccctccctgtcaccgccctgctgatccgctggctcactgctccacgccgctcgg |
| Full-nt | | cccgagattcagctcgtgcaatcgggagcggaagtcaagaagccaggagagtccagcg |
| | | gatctcatgcaagggtagcggattaacatcgaggattactacatccactgggtgaggc |
| | | agatgccggggaagggactcgaatggatgggacggatcgacccagaaaacgacgaaac |
| | | taagtacggtccgatcaccaaggccatgtgactattagcgccgatacttcaatcaata |
| | | ccgtgtatctgcaatggtcctcattgaaagcctcagataccgcgatgtactactgtga |
| | | ttcagaggaggggtctactggggacagggaactaccgtgactgtctcgtccggcggag |
| | | gcgggtcaggaggtggcggcagcggaggaggagggtccggcggaggtgggtccgacgt |
| | | cgtgatgacccagagccctgacagcctggcagtgagcctgggcgaaagagctaccatt |
| | | aactgcaaatcgtcgcagagcctgctggactcggacggaaaaacgtacctcaattggc |
| | | tgcagcaaaagcctggccagccaccgaagcgcatatctcactggtgtcgaagctggat |
| | | tcgggagtgcccgatcgcactccggctcgggatcgggtactgacttcaccctcactat |
| | | ctcctcgcttcaagcagaggacgtggccgtctactactgctggcagggaacccactac |
| | | cgggaaccacggcggagggacgaaagtggagatcaagaccactaccccagcaccgagg |
| | | ccacccaccccggctcctaccatcgcctcccagcctctgtccctgcgtccggaggcat |
| | | gtagacccgcagctggtggggccgtgcatacccggggtcttgacttcgcctgcgatat |
| | | ctacatagggcccctctggctggtacttgcggggtcctgctgattcactcgtgatcac |
| | | tctttactgtaagcgcggtcggaagaagctgctgtacatattaagcaaccatcatgag |
| | | gcctgtgcagactactcaagaggaggacggctgacatgccggacccagaggaggagga |
| | | aggcggctgcgaactgcgcgtgaaattcagccgcagcgcagatgctccagcctacaag |
| | | caggggcagaaccagctctacaacgaactcaatcttggtcggagagaggagtacgacg |
| | | tgctggacaagcggagaggacgggacccagaaatgggcgggaagccgcgcagaaagaa |
| | | tccccaagagggcctgtacaacgagctccaaaaggataagatggcagaagcctatagc |
| | | gagattggtatgaaaggggaacgcagaagaggcaaaggccacgacggactgtaccagg |
| | | gactcagcaccgccaccaaggacacctatgacgctcttcacatgcaggccctgccgcc |
| | | tcgg |
| CAR6- | 73 | malpvtalllplalllhaarpqeilvqsgaevkkpgeslrisckgsgfniedyyihwv |
| Full-aa | | rqmpgkglewmgridpendetkygpifqghvtisadtsintvylqwsslkasdtamyy |
| | | cafrggvywgqgttvtvssggggsggggsggggsggggsdvvmtqspdslavslgera |
| | | tinckssqslldsdgktylnwlqqkpgqppkrlislvskldsgvpdrfsgsgsgtdft |
| | | ltisslqaedvavyycwqgthfpgtfgggtkveiktttpaprpptpaptiasqplslr |
| | | peacrpaaggavhtrgldfacdiyiwaplagtcgvlllslvitlyckrgrkkllyifk |
| | | qpfmrpvqttqeedgcscrfpeeeeggcelrvkgsrsadapaykqgqnqlynelnlgr |
| | | reeydvldkrrgrdpemggkprrknpqelgynelqkdkmaeayseigmkgerrrgkgh |
| | | dglyqglstatdtydalhmqalppr |

CAR 7

| | | |
|---|---|---|
| CAR7 | 74 | dvvmtqspdslavslgeratinckssqslldsdgkgylnwlqqkpgqppkrlislvsk |
| scFv | | ldsgvpdrfsgsgsgtdftltisslqaedvavyycwqghtfpgtfgggtkveikgggg |
| domain | | sggggsggggsggggseiqlvqsgaevkkpgeslrisckgsgfniedyyihwvrqmpg |
| | | kglewmgridpendetkygpifqghvtisadtsintvylqwsslkasdtamyycafrg |
| | | gvywgqgttvtvss |
| CAR7 | 75 | gacgtggtgatgacccaatcgccagattccctggcagtgtccctgggcgaacgcgcca |
| scFv | | ctattaactgcaaatcgtcacagtccttgcttgattccgacggaaagacctacctcaa |
| domain nt | | ttggctccagcagaagccaggacaaccgccaaagagactgatctccctggtgtcaaag |
| | | ctggactcgggagtgcctgatcggttctcgggtagcgggagcggcaccgacttcactc |
| | | tgaccatctcgtcactccaggctgaggacgtggccgtgtattactgttggcagggtac |
| | | tcactttccgggcactttcggaggcggcaccaaggtggagattaaaggaggaggcgga |
| | | agcggaggtggaggatcgggaggtggtgggagcggcggaggagggagcgagatccagc |
| | | tcgtccaatcgggagcggaagtgaagaagcccggagagtcacttagaatctcatgcaa |
| | | ggggtcgggcttcaacatcgaggattactacatccattgggtccgccagatgcctggt |
| | | aaaggactggaatggatggggaggattgacccggaaaacgacgaaactaagtacggac |
| | | cgatctttcaagggcacgtgactatctccgctgatacctcaatcaatactgtctacct |
| | | ccagtggtcctcgctgaaagcaagcgacaccgcgatgtactactgcgccttccgggga |
| | | ggagtgtactggggccaaggcaccacggtcacggtcagctcc |
| CAR7- | 76 | atggccctccctgtcaccgccctgctgcttccgctggctcttctgctccacgccgctc |
| Soluble | | ggcccgacgtggtgatgacccaatcgccagattccctggcagtgtccctgggcgaacg |
| scFv-nt | | cgccactattaactgcaaatcgtcacagtccttgcttgattccgacggaaagacctac |
| | | ctcaattggctccagcagaagccaggacaaccgccaaagagactgatctccctggtgt |
| | | caaagctggactcgggagtgcctgatcggttctcgggtagcgggagcggcaccgactt |
| | | cactctgaccatctcgtcactccaggctgaggacgtggccgtgtattactgttggcag |
| | | ggtactcactttccgggcactttcggaggcggcaccaaggtggagattaaaggaggag |
| | | gcggaagcggaggtggaggatcgggaggtggtgggagcggcggaggagggagcgagat |
| | | ccagctcgtccaatcgggagcggaagtgaagaagcccggagagtcacttagaatctca |
| | | tgcaaggggtcgggcttcaacatcgaggattactacatccattgggtccgccagatgc |
| | | ctggtaaaggactggaatggatggggaggattgacccggaaaacgacgaaactaagta |
| | | cggaccgatctttcaagggcacgtgactatctccgctgatacctcaatcaatactgtc |
| | | tacctccagtggtcctcgctgaaagcaagcgacaccgcgatgtactactgcgccttcc |
| | | ggggaggagtgtactggggccaaggcaccacggtcacggtcagctccggctcccatca |
| | | ccaccaccatcaccatcatcac |
| CAR7- | 77 | malpvtalllplalllhaarpdvvmtqspdslavlsgeratinckssqslldsdgkty |
| Soluble | | lnwlqqkpgqppkrlislvskldsgvpdrfsgsgsgtdftltisslqaedvavyycwq |
| scFv-aa | | gthfpgtfgggtkveikggggsggggsggggsggggseiqlvqsgaevkkpgeslris |
| | | ckgsgfniedyyihwvrqmpgkglewmgridpendetkygpifqghvtisadtsintv |
| | | ylqwsslkasdtamyycafrggvywgqgttvtvssgshhhhhhhh |
| CAR 7 | 78 | atggccctccctgtcaccgccctgctgcttccgctggctcttctgctccacgccgctc |
| Full-nt | | ggcccgacgtggtgatgactcagtcgcctgactcgctggctgtgtcccttggagagcg |
| | | ggccactatcaattgcaagtcatcccagtcgctgctggattccgacgggaaaacctac |
| | | ctcaattggctgcagcaaaaaccgggacagcctccaaagcggctcatcagcctggtgt |
| | | ccaagttggacagcggcgtgccagaccgcttctccggttcgggaagcggtactgattt |
| | | cacgctgaccatctcatccctccaagcggaggatgtggcagtctactactgttggcag |
| | | ggcacgcattttccgggcacttttggaggagggaccaaggtcgaaatcaagggaggag |
| | | gtggctcgggcggaggaggctcgggaggaggaggatcaggaggcggtggaagcgagat |
| | | tcaactggtccagagcggcgcagaagtcaagaagccgggtgaatcgctcagaatctcg |
| | | tgcaaaggatcgggattcaacatcgaggactactacattcactgggtcagacaaatgc |
| | | cgggcaaagggctggaatggatggggaggatcgaccccgaaaacgatgaaaccaagta |
| | | cggaccaatcttccaagggcacgtgaccatttcggcggacacctcaatcaacactgtg |
| | | tacctccagtggagctcacttaaggccagcgataccgccatgtactattgcgctttcc |
| | | gcggaggggtgtactggggacagggcactactgtgaccgtgtcatccaccactacccc |
| | | agcaccgaggccacccaccccggctcctaccatcgcctcccagcctctgtccctgcgt |
| | | ccggaggcatgtagacccgcagctggtggggccgtgcatacccggggtcttgacttcg |
| | | cctgcgatatctacatttgggcccctctggctggtacttgcggggtcctgctgctttc |
| | | actcgtgatcactctttactgtaagcgcggtcggaagaagctgctgtacatctttaag |
| | | caacccttcatgaggcctgtgcagactactcaagaggaggacggctgttcatgccggt |
| | | tcccagaggaggaggaaggcggctgcgaactgcgcgtgaaattcagccgcagcgcaga |
| | | tgctccagcctacaagcaggggcagaaccagctctacaacgaactcaatcttggtcgg |
| | | agagaggagtacgacgtgctggacaagcggagaggacgggacccagaaatgggcggga |
| | | agccgcgcagaaagaatccccaagagggcctgtacaacgagctccaaaaggataagat |
| | | ggcagaagcctatagcgagattggtatgaaaggggaacgcagaagaggcaaaggccac |
| | | gacggactgtaccagggactcagcaccgccaccaaggacacctatgacgctcttcaca |
| | | tgcaggccctgccgcctcgg |
| CAR 7 | 79 | malpvtalllplalllhaarpdvvmtqspdslavslgeratinckssqslldsdgkty |
| Full-aa | | lnwlqqkpgqppkrlislvskldsgvpdrfsgsgsgtdftltisslqaedvavyycwq |
| | | gthfpgtfgggtkveikggggsggggsggggsggggseiqlvqsgaevkkpgeslris |
| | | ckgsgfniedyvhiwvrqmpgkglewmgridpendetkvgpifqghvtisadtsintv |
| | | ylqwsslkasdtamyycafrggvywgqgttvtvsstttpaprpptpaptiasqplslr |
| | | peacrpaaggavhtrgldfacdiyiwaplagtcgvlllslvitlyckrgrkkllyifk |
| | | qpfmrpvqttqeedgcscrfpeeeeggcelrvkfsrsadapaykqgqnqlynelnlgr |
| | | reeydvldkrrgrdpemggkprrknpqeglynelqkdkmaeayseigmkgerrrgkgh |
| | | dglyqglstatdtydalhmqalppr |

CAR 8

| | | |
|---|---|---|
| CAR8 | 80 | dvvmtqsplslpvtlgqpasisckssqslldsdgktylnwlqqrpgqsprrlislvsk |
| scFv | | ldsgvpdrfsgsgsgtdftltkisrveaedvgvyycwqgthfpgtfgggtkveikggg |
| domain | | gsggggsggggsggggseiqlvqsgaevkkpgatvkisckgsgfniedyyihwvqqap |
| | | gkglewmgridpendetkygpifqgrvtitadtstntvymelsslrsedtavyycafr |
| | | ggvywgqgttvtvss |
| CAR8 | 81 | gatgtggtcatgacgcagtcaccactgtccctccccgtgacccttggacagccagcgt |
| scFv | | cgattagctgcaagtcatcccaatccctgctcgattcggatggaaagacctatctcaa |
| domain nt | | ctggctgcagcaaagacccggtcagagccctaggagactcatctcgttggtgtcaaag |
| | | ctggacagcggagtgccggaccggttttccggttcgggatcggggacggacttcactc |
| | | tgaagatttcacgggtggaagctgaggatgtgggagtgtactactgctggcagggaac |
| | | ccatttccctggcacttttggcggaggaactaaggtcgaaatcaagggaggaggtggc |
| | | tcgggaggaggcggatcgggcggaggcgggagcggcggaggagggtccgaaatccaac |
| | | ttgtccagtcaggagccgaagtgaagaaaccgggagccaccgtcaaaatcagctgtaa |
| | | gggatcgggattcaatatcgaggactactacatccactgggtgcagcaagctccgggc |
| | | aaaggactggagtggatggggcgcatcgacccagagaacgacgaaaccaaatacggcc |
| | | cgatcttccaagggcgggtgaccatcaccgcggacacctcaactaacactgtgtacat |
| | | ggagctgagctccctgcgctccgaagatactgcagtctactactgcgccttccgcggt |
| | | ggtgtgtactggggacagggcaccactgtgactgtcagctcg |
| CAR8- | 82 | atggccctccctgtcaccgccctgctgcttccgctggctcttctgctccacgccgctc |
| Soluble | | ggcccgatgtggtcatgacgcagtcaccactgtccctccccgtgacccttggacagcc |
| scFv-nt | | agcgtcgattagctgcaagtcatcccaatccctgctcgattcggatggaaagacctat |
| | | ctcaactggctgcagcaaagacccggtcagagccctaggagactcatctcgttggtgt |
| | | caaagctggacagcggagtgccggaccggttttccggttcgggatcggggacggactt |
| | | cactctgaagatttcacgggtggaagctgaggatgtgggagtgtactactgctggcag |
| | | ggaacccatttccctggcacttttggcggaggaactaaggtcgaaatcaagggaggag |
| | | gtggctcgggaggaggcggatcgggcggaggcgggagcggcggaggagggtccgaaat |
| | | ccaacttgtccagtcaggagccgaagtgaagaaaccgggagccaccgtcaaaatcagc |
| | | tgtaagggatcgggattcaatatcgaggactactacatccactgggtgcagcaagctc |
| | | cgggcaaaggactggagtggatggggcgcatcgacccagagaacgacgaaaccaaata |
| | | cggcccgatcttccaagggcgggtgaccatcaccgcggacacctcaactaacactgtg |
| | | tacatggagctgagctccctgcgctccgaagatactgcagtctactactgcgccttcc |
| | | gcggtggtgtgtactggggacagggcaccactgtgactgtcagctcggggtcccacca |
| | | tcatcaccaccaccatcac |
| CAR8- | 83 | malpvtalllplalllhaarpdvvmtqsplslpvtlgqpasisckssqslldsdgkty |
| Soluble | | lnwlqqrpgqsprrlislvskldsgvpdrfsgsgsgtdftlisrveaedvgvyycwqg |
| scFv-aa | | thfpgtfgggtkveikggggsggggsggggsggggseiqlvqsgaevkkpgatvkisc |
| | | kgsfgniedyyihwvqqapgkglewmgridpendetkygpifqgrvtitadtstntvy |
| | | melsslrsedtavyycafrggvywgqgttvtvssgshhhhhhhh |
| CAR 8- | 84 | atggccctccctgtcaccgccctgctgcttccgctggctcttctgctccacgccgctc |
| Full-nt | | ggcccgatgtggtcatgacgcagtcaccactgtccctccccgtgacccttggacagcc |
| | | agcgtcgattagctgcaagtcatcccaatccctgctcgattcggatggaaagacctat |
| | | ctcaactggctgcagcaaagacccggtcagagccctaggagactcatctcgttggtgt |
| | | caaagctggacagcggagtgccggaccggttttccggttcgggatcggggacggactt |
| | | cactctgaagatttcacgggtggaagctgaggatgtgggagtgtactactgctggcag |
| | | ggaacccatttccctggcacttttggcggaggaactaaggtcgaaatcaagggaggag |
| | | gtggctcgggaggaggcggatcgggcggaggcgggagcggcggaggagggtccgaaat |
| | | ccaacttgtccagtcaggagccgaagtgaagaaaccgggagccaccgtcaaaatcagc |
| | | tgtaagggatcgggattcaatatcgaggactactacatccactgggtgcagcaagctc |
| | | cgggcaaaggactggagtggatggggcgcatcgacccagagaacgacgaaaccaaata |
| | | cggcccgatcttccaagggcgggtgaccatcaccgcggacacctcaactaacactgtg |
| | | tacatggagctgagctccctgcgctccgaagatactgcagtctactactgcgccttcc |
| | | gcggtggtgtgtactggggacagggcaccactgtgactgtcagctcgaccactacccc |
| | | agcaccgaggccacccaccccggctcctaccatcgcctcccagcctctgtccctgcgt |
| | | ccggaggcatgtagacccgcagctggtggggccgtgcatacccggggtcttgacttcg |
| | | cctgcgatatctacatttgggcccctctggctggtacttgcggggtcctgctgctttc |
| | | actcgtgatcactciltactgtaagcgcggtcggaagaagctgctgtacatctttaag |
| | | caacccttcatgaggcctgtgcagactactcaagaggaggacggctgttcatgccggt |
| | | tcccagaggaggaggaaggcggctgcgaactgcgcgtgaaattcagccgcagcgcaga |
| | | tgctccagcctacaagcaggggcagaaccagctctacaacgaactcaatcttggtcgg |
| | | agagaggagtacgacgtgctggacaagcggagaggacgggacccagaaatgggcggga |
| | | agccgcgcagaaagaatccccaagagggcctgtacaacgagctccaaaaggataagat |
| | | ggcagaagcctatagcgagattggtatgaaaggggaacgcagaagaggcaaaggccac |
| | | gacggactgtaccagggactcagcaccgccaccaaggacacctatgacgctcttcaca |
| | | tgcaggccctgccgcctcgg |
| CAR 8- | 85 | malpvtalllplalllhaarpdvvmtqsplslpvtlgqpasisckssqslldsdgkty |
| Full-aa | | lnwlqqrpgqsprrlislvskldsgvpdrfsgsgsgtdftlkisrveaedvgvyycwq |
| | | gthfpgtfgggtkveikggggsggggsggggsggggseiqlvqsgaevkkpgatvkis |
| | | ckgsgfniedyyihwvqqapgklgewmgridpendetkygpifqgrvtitadtstntv |
| | | ymelsslrsedtavyycafrggvywgqgttvtvsstttpaprpptpaptiasqplslr |
| | | peacrpaaggavhtrgldfacdiyiwaplagtcgvlllslvitlyckrgrkkllyifk |
| | | qpfmrpvqttqeedgcscrfpeeeeggcelrvkfsrsadapaykqgqnqlynelnlgr |
| | | reeydvldkrrgrdpemggkprrknpqeglynelqkdkmaeayseigmkgerrrgkgh |
| | | dglyqglstatkdtydalhmqalppr |

CAR 9 Mouse anti-EGFRvIII clone 3C10

| | | |
|---|---|---|
| CAR9 | 86 | eiqlqqsgaelvkpgasvklsctgsgfniedyyihwvkqrteqglewigridpendet |
| scFv | | kygpifqgratitadtssntvylqlssltsedtavyycafrggvywgpgttlvtvssg |
| domain | | gggsgggsggggshmdvvmtqspltlsvaigqsasisckssqslldsdgktylnwllq |
| | | rpgqspkrlislvskldsgvpdrftgsgsgtdftlrisrveaedlgiyycwqghtfpg |
| | | tfgggtkleik |
| CAR9 | 98 | gagatccagctccaacagagcggagccgaactggtcaaaccgggagcgtcggtgaagt |
| scFv | | tgtcatgcactggatcgggcttcaacatcgaggattactacatccactgggtcaagca |
| domain nt | | acgcaccgagcaggggctggaatggatcggacggatcgaccccgaaaacgatgaaacc |
| | | aagtacgggcctatcttccaaggacgggccaccattacggctgacacgtcaagcaata |
| | | ccgtctacctccagctttccagcctgacctccgaggacactgccgtgtactactgcgc |
| | | cttcagaggaggcgtgtactggggaccaggaaccactttgaccgtgtccagcggaggc |
| | | ggtggatcaggaggaggaggctcaggcggtggcggctcgcacatggacgtggtcatga |
| | | ctcagtccccgctgaccctgtcggtggcaattggacagagcgcatccatctcgtgcaa |
| | | gagctcacagtcgctgctggattccgacggaaagacttatctgaactggctgctccaa |
| | | agaccagggcaatcaccgaaacgccttatctccctggtgtcgaaactcgactcgggtg |
| | | tgccggatcggtttaccggtagcgggtccggcacggacttcactctccgcatttcgag |
| | | ggtggaagcggaggatctcgggatctactactgttggcagggaacccacttccctggg |
| | | acttttggaggcggaactaagctggaaatcaag |
| CAR9- | 87 | atggccctccctgtcaccgccctgctgcttccgctggctcttctgctccacgccgctc |
| Soluble | | ggcccgagatccagctccaacagagcggagccgaactggtcaaaccgggagcgtcggt |
| scFv-nt | | gaagttgtcatgcactggatcgggcttcaacatcgaggattactacatccactgggtc |
| | | aagcaacgcaccgagcaggggctggaatggatcggacggatcgaccccgaaaacgatg |
| | | aaaccaagtacgggcctatcttccaaggacgggccaccattacggctgacacgtcaag |
| | | caataccgtctacctccagctttccagcctgacctccgaggacactgccgtgtactac |
| | | tgcgccttcagaggaggcgtgtactggggaccaggaaccactttgaccgtgtccagcg |
| | | gaggcggtggatcaggaggaggaggctcaggcggtggcggctcgcacatggacgtggt |
| | | catgactcagtccccgctgaccctgtcggtggcaattggacagagcgcatccatctcg |
| | | tgcaagagctcacagtcgctgctggattccgacggaaagacttatctgaactggctgc |
| | | tccaaagaccagggcaatcaccgaaacgccttatctccctggtgtcgaaactcgactc |
| | | gggtgtgccggatcggtttaccggtagcgggtccggcacggacttcactctccgcatt |
| | | tcgagggtggaagcggaggatctcgggatctactactgttggcagggaacccacttcc |
| | | ctgggacttttggaggcggaactaagctggaaatcaagggtagccatcaccatcacca |
| | | ccaccatcat |
| CAR9- | 88 | malpvtalllplalllhaarpeiqlqqsgaelvkpgasvklsctgsgfneidyyihwv |
| Soluble | | kqrteqglewigridpendetkygpifqgratitadtssntvylqlssltsedtavyy |
| scFv-aa | | cafrggvywgpgttlvtvssggggsggggsggggshmdvvmtqspltlsvaigqsasi |
| | | sckssqslldsdgktylnwllqrpgqspkrlislvskldsgvpdrftgsgsgtdftlr |
| | | isrveaedlgiyycwqgthfpgtfgggtkleikgshhhhhhhh |
| CAR 9- | 89 | atggccctccctgtcaccgccctgctgcttccgctggctcttctgctccacgccgctc |
| Full-nt | | ggcccgagatccagctccaacagagcggagccgaactggtcaaaccgggagcgtcggt |
| | | gaagttgtcatgcactggatcgggcttcaacatcgaggattactacatccactgggtc |
| | | aagcaacgcaccgagcaggggctggaatggatcggacggatcgaccccgaaaacgatg |
| | | aaaccaagtacgggcctatcttccaaggacgggccaccattacggctgacacgtcaag |
| | | caataccgtctacctccagctttccagcctgacctccgaggacactgccgtgtactac |
| | | tgcgccttcagaggaggcgtgtactggggaccaggaaccactttgaccgtgtccagcg |
| | | gaggcggtggatcaggaggaggaggctcaggcggtggcggctcgcacatggacgtggt |
| | | catgactcagtccccgctgaccctgtcggtggcaattggacagagcgcatccatctcg |
| | | tgcaagagctcacagtcgctgctggattccgacggaaagacttatctgaactggctgc |
| | | tccaaagaccagggcaatcaccgaaacgccttatctccctggtgtcgaaactcgactc |
| | | gggtgtgccggatcggtttaccggtagcgggtccggcacggacttcactctccgcatt |
| | | tcgagggtggaagcggaggatctcgggatctactactgttggcagggaacccacttcc |
| | | ctgggacttttggaggcggaactaagctggaaatcaagaccactaccccagcaccgag |
| | | gccacccaccccggctcctaccatcgcctcccagcctctgtccctgcgtccggaggca |
| | | tgtagacccgcagctggtggggccgtgcatacccggggtcttgacttcgcctgcgata |
| | | tctacatttgggcccctctggctggtacttgcggggtcctgctgctttcactcgtgat |
| | | cactctttactgtaagcgcggtcggaagaagctgctgtacatctttaagcaacccttc |
| | | atgaggcctgtgcagactactcaagaggaggacggctgttcatgccggttcccagagg |
| | | aggaggaaggcggctgcgaactgcgcgtgaaattcagccgcagcgcagatgctccagc |
| | | ctacaagcaggggcagaaccagctctacaacgaactcaatcttggtcggagagaggag |
| | | tacgacgtgctggacaagcggagaggacgggacccagaaatgggcgggaagccgcgca |
| | | gaaagaatccccaagagggcctgtacaacgagctccaaaaggataagatggcagaagc |
| | | ctatagcgagattggtatgaaaggggaacgcagaagaggcaaaggccacgacggactg |
| | | taccagggactcagcaccgccaccaaggacacctatgacgctcttcacatgcaggccc |
| | | tgccgcctcgg |
| CAR 9- | 90 | malpvtalllplalllhaarpeiqlqqsgaelvkpgasvklsctgsgfniedyyihwv |
| Full-aa | | kqrtegqlewigridpendetkygpirqgratitadtssntvylqlssltsedtavyy |
| | | cafrggvywgpgttltvssggggsggggsggggshmdvvmtqspltlsvaigqsasis |
| | | ckssqslldsdgktylnwllqrpgqspkrlislvskldsgvpdrftgsgsgtdftlri |
| | | srveaedlgiyycwqgthfpgtfgggtkleiktttpaprpptpaptiasqplslrpea |
| | | crpaaggavhtrgldfacdiyiwaplagtcgvlllslvitlyckrgrkkllyifkqpf |
| | | mrpvqttqeedgcscrfpeeeeggcelrvkfsrsadapaykqgqnqlynelnlgrree |
| | | ydvldkrrgrdpemggkprrknpqeglynelqkdkmaeayseigmkgerrrgkghdgl |
| | | yqglstatkdtydalhmqalppr |

CAR10 Anti-EGFRvIII clone 139

| | | |
|---|---|---|
| CAR10 | 91 | diqmtqspsslsasvgdrvtitcrasqgirnnlawyqqkpgkapkrliyaasnlqsgv |
| scFv | | psrftgsgsgteftlivsslqpedfatyyclqhhsypltsgggtkveikrtgstsgsg |
| domain | | kpgsgegsevqvlesggglvqpggslrlscaasgftfssyamswvrqapgkglewvsa |
| | | isgsggstnyadsvkgrftisrdnskntlylqmnslraedtavyycagssgwseywgq |
| | | gtlvtvss |
| CAR9 | 92 | gatatccaaatgactcagagcccttcatccctgagcgccagcgtcggagacagggtga |
| scFv | | ccatcacgtgccgggcatcccaaggcattagaaataacttggcgtggtatcagcaaaa |
| domain nt | | accaggaaaggccccgaagcgcctgatctacgcggcctccaaccttcagtcaggagtg |
| | | ccctcgcgcttcaccgggagcggtagcggaactgagtttacccttatcgtgtcgtccc |
| | | tgcagccagaggacttcgcgacctactactgcctccagcatcactcgtacccgttgac |
| | | ttcgggaggcggaaccaaggtcgaaatcaaacgcactggctcgacgtcagggtccggt |
| | | aaaccgggatcgggagaaggatcggaagtccaagtgctggagagcggaggcggactcg |
| | | tgcaacctggcgggtcgctgcggctcagctgtgccgcgtcgggttttactttcagctc |
| | | gtacgctatgtcatgggtgcggcaggctccgggaaaggggctggaatgggtgtccgct |
| | | atttccggctcgggtggaagcaccaattacgccgactccgtgaagggacgcttcacca |
| | | tctcacgggataactccaagaatactctgtacctccagatgaactcgctgagagccga |
| | | ggacaccgcagtgtactactgcgcagggtcaagcggctggtccgaatactggggacag |
| | | ggcaccctcgtcactgtcagctcc |
| CAR10- | 93 | atggccctccctgtcaccgccctgctgcttccgctggctcttctgctccacgccgctc |
| Soluble | | ggcccgatatccaaatgactcagagcccttcatccctgagcgccagcgtcggagacag |
| scFv-nt | | ggtgaccatcacgtgccgggcatcccaaggcattagaaataacttggcgtggtatcag |
| | | caaaaaccaggaaaggccccgaagcgcctgatctacgcggcctccaaccttcagtcag |
| | | gagtgccctcgcgcttcaccgggagcggtagcggaactgagtttacccttatcgtgtc |
| | | gtccctgcagccagaggacttcgcgacctactactgcctccagcatcactcgtacccg |
| | | ttgacttcgggaggcggaaccaaggtcgaaatcaaacgcactggctcgacgtcagggt |
| | | ccggtaaaccgggatcgggagaaggatcggaagtccaagtgctggagagcggaggcgg |
| | | actcgtgcaacctggcgggtcgctgcggctcagctgtgccgcgtcgggttttactttc |
| | | agctcgtacgctatgtcatgggtgcggcaggctccgggaaaggggctggaatgggtgt |
| | | ccgctatttccggctcgggtggaagcaccaattacgccgactccgtgaagggacgctt |
| | | caccatctcacgggataactccaagaatactctgtacctccagatgaactcgctgaga |
| | | gccgaggacaccgcagtgtactactgcgcagggtcaagcggctggtccgaatactggg |
| | | gacagggcaccctcgtcactgtcagctcccatcaccatcaccaccaccatcac |
| CAR10- | 94 | malpvtalllplalllhaarpdqimtqspsslsasvgdrvtitcrasqgirnnlawyq |
| Soluble | | qkpgkapkrliyaasnlqsgvpsrftgsgsgteftlivsslqpedfatyyclqhhsyp |
| scFv-aa | | ltsgggtkveikrtgstsgsgkpgsgegsevqvlesggglvqpggslrlscaasgftf |
| | | ssyamswvrqapgkglewvsaisgsggstnyadsvkgrftisrdnskntlylmqnslr |
| | | aedtavyycagssgwseywgqgtlvtvsshhhhhhhh |
| CAR 10 | 95 | atggccctccctgtcaccgccctgctgcttccgctggctcttctgctccacgccgctc |
| Full-nt | | ggcccgatatccaaatgactcagagcccttcatccctgagcgccagcgtcggagacag |
| | | ggtgaccatcacgtgccgggcatcccaaggcattagaaataacttggcgtggtatcag |
| | | caaaaaccaggaaaggccccgaagcgcctgatctacgcggcctccaaccttcagtcag |
| | | gagtgccctcgcgcttcaccgggagcggtagcggaactgagtttacccttatcgtgtc |
| | | gtccctgcagccagaggacttcgcgacctactactgcctccagcatcactcgtacccg |
| | | ttgacttcgggaggcggaaccaaggtcgaaatcaaacgcactggctcgacgtcagggt |
| | | ccggtaaaccgggatcgggagaaggatcggaagtccaagtgctggagagcggaggcgg |
| | | actcgtgcaacctggcgggtcgctgcggctcagctgtgccgcgtcgggttttactttc |
| | | agctcgtacgctatgtcatgggtgcggcaggctccgggaaaggggctggaatgggtgt |
| | | ccgctatttccggctcgggtggaagcaccaattacgccgactccgtgaagggacgctt |
| | | caccatctcacgggataactccaagaatactctgtacctccagatgaactcgctgaga |
| | | gccgaggacaccgcagtgtactactgcgcagggtcaagcggctggtccgaatactggg |
| | | gacagggcaccctcgtcactgtcagctccaccactaccccagcaccgaggccacccac |
| | | cccggctcctaccatcgcctcccagcctctgtccctgcgtccggaggcatgtagaccc |
| | | gcagctggtggggccgtgcatacccggggtcttgacttcgcctgcgatatctacattt |
| | | gggcccctctggctggtacttgcggggtcctgctgctttcactcgtgatcactcttta |
| | | ctgtaagcgcggtcggaagaagctgctgtacatctttaagcaacccttcatgaggcct |
| | | gtgcagactactcaagaggaggacggctgttcatgccggttcccagaggaggaggaag |
| | | gcggctgcgaactgcgcgtgaaattcagccgcagcgcagatgctccagcctacaagca |
| | | ggggcagaaccagctctacaacgaactcaatcttggtcggagagaggagtacgacgtg |
| | | ctggacaagcggagaggacgggacccagaaatgggcgggaagccgcgcagaaagaatc |
| | | cccaagagggcctgtacaacgagctccaaaaggataagatggcagaagcctatagcga |
| | | gattggtatgaaaggggaacgcagaagaggcaaaggccacgacggactgtaccaggga |
| | | ctcagcaccgccaccaaggacacctatgacgctcttcacatgcaggccctgccgcctc |
| | | gg |
| CAR 10 | 96 | malpvtalllplalllhaarpdiqmtqspsslsasvgdrvtitcrasqgirnnlawyq |
| Full-aa | | qkpgkapkrliyaasnlqsgvpsrftgsgsgteftlivsslqpedfatyyclqhhsyp |
| | | ltsgggtkveikrtgstsgsgkpgsgegsevqvlesggglvqpggslrlscaasgftf |
| | | ssyamswvrqapgkglewvsaisgsggstnyadsvkgrftisrdnskntlylqmnslr |
| | | aedtavyycagssgwseywgqgtlvtvsstttpaprpptpaptiasqplslrpeacrp |
| | | aaggavhtrgldfacdiyiwaplagtcgvlllslvitlyckrgrkkllyifqpfmrpv |
| | | qttqeedgcscrfpeeeeggcelrvkfsrsadapaykqgqnqlynelnlgrreeydvl |
| | | dkrrgrdpemggkprrknpqeglynelqkdkmaeayseigmkgerrrgkghdglygql |
| | | statkdtydalhmqalppr |

CAR1_VH

| | | |
|---|---|---|
| SEQ ID NO: 537 | HCDR1 | GFNIEDYY |
| (IMGT) | | |
| SEQ ID NO: 538 | HCDR2 | IDPENDET |
| (IMGT) | | |
| SEQ ID NO: 539 | HCDR3 | AFRGGVY |
| (IMGT) | | |
| SEQ ID NO: 123 | VH | EIQLVQSGAEVKKPGATVKISCKGSGFNIEDYYIHWVQQAPGKGLEWMGRIDPENDET |
| | | KYGPIFQGRVTITADTSTNTVYMELSSLRSEDTAVYYCAFRGGVYWGQGTTVTVSS |

CAR1_VL

| | | |
|---|---|---|
| SEQ ID NO: 540 | LCDR1 | QSLLDSDGKTY |
| (IMGT) | | |
| SEQ ID NO: 35 | LCDR2 | LVS |
| (IMGT) | | |
| SEQ ID NO: 28 | LCDR3 | WQGTHFPGT |
| (IMGT) | | |
| SEQ ID NO: 127 | VL | DVVMTQSPDSLAVSLGERATINCKSSQSLLDSDGKTYLNWLQQKPGQPPKRLISLVSK |
| | | LDSGVPDRFSGSGSGTDFTLTISSLQAEDVAVYYCWQGTHFPGTFGGGTKVEIK |

CAR2_VL

| | | |
|---|---|---|
| SEQ ID NO: 540 | LCDR1 | QSLLDSDGKTY |
| (IMGT) | | |
| SEQ ID NO: 35 | LCDR2 | LV2 |
| (IMGT) | | |
| SEQ ID NO: 28 | LCDR3 | WQGTHFPGT |
| (IMGT) | | |
| SEQ ID NO: 127 | VL | DVVMTQSPDSLAVSLGERATINCKSSQSLLDSDGKTYLNWLQQKPGQPPKRLISLVSK |
| | | LDSGVPDRFSGSGSGTDFTLTISSLQAEDVAVYYCWQGTHFPGTFGGGTKVEIK |

CAR2_VH

| | | |
|---|---|---|
| SEQ ID NO: 537 | HCDR1 | GFNIEDYY |
| (IMGT) | | |
| SEQ ID NO: 538 | HCDR2 | IDPENDET |
| (IMGT) | | |
| SEQ ID NO: 539 | HCDR3 | AFRGGVY |
| (IMGT) | | |
| SEQ ID NO: 123 | VH | EIQLVQSGAEVKKPGATVKISCKGSGFNIEDYYIHWVQQAPGKGLEWMGRIDPENDET |
| | | KYGPIFQGRVTITADTSTNTVYMELSSLRSEDTAVYYCAFRGGVYWGQGTTVTVSS |

CAR3_VH

| | | |
|---|---|---|
| SEQ ID NO: 537 | HCDR1 | GFNIEDYY |
| (IMGT) | | |
| SEQ ID NO: 538 | HCDR2 | IDPENDET |
| (IMGT) | | |
| SEQ ID NO: 539 | HCDR3 | AFRGGVY |
| (IMGT) | | |
| SEQ ID NO: 124 | VH | EIQLVQSGAEVKKPGESLRISCKGSGFNIEDYYIHWVRQMPGKGLEWMGRIDPENDET |
| | | KYGPIFQGHVTISADTSINTVYLQWSSLKASDTAMYYCAFRGGVYWGQGTTVTVSS |

CAR3_VL

| | | |
|---|---|---|
| SEQ ID NO: 540 | LCDR1 | QSLLDSDGKTY |
| (IMGT) | | |
| SEQ ID NO: 35 | LCDR2 | LVS |
| (IMGT) | | |
| SEQ ID NO: 28 | LCDR3 | WQGTHFPGT |
| (IMGT) | | |
| SEQ ID NO: 126 | VL | DVVMTQSPLSLPVTLGQPASISCKSSQSLLDSDGKTYLNWLQQRPGQSPRRLISLVSK |
| | | LDSGVPDRFSGSGSGTDFTLKISRVEAEDVGVYYCWQGTHFPGTFGGGTKVEIK |

CAR4_VL

| | | |
|---|---|---|
| SEQ ID NO: 540 | LCDR1 | QSLLDSDGKTY |
| (IMGT) | | |
| SEQ ID NO: 35 | LCDR2 | LVS |
| (IMGT) | | |
| SEQ ID NO: 28 | LCDR3 | WQGTHFPGT |
| (IMGT) | | |
| SEQ ID NO: 126 | VL | DVVMTQSPLSLPVTLGQPASISCKSSQSLLDSDGKTYLNWLQQRPGQSPRRLISLVSK |
| | | LDSGVPDRFSGSGSGTDFTLKISRVEAEDVGVYYCWQGTHFPGTFGGGTKVEIK |

CAR4_VH

| | | |
|---|---|---|
| SEQ ID NO: 537 | HCDR1 | GFNIEDYY |
| (IMGT) | | |
| SEQ ID NO: 538 | HCDR2 | IDPENDET |
| (IMGT) | | |
| SEQ ID NO: 539 | HCDR3 | AFRGGVY |
| (IMGT) | | |
| SEQ ID NO: 124 | VH | EIQLVQSGAEVKKPGESLRISCKGSGFNIEDYYIHWVRQMPGKGLEWMGRIDPENDET |
| | | KYGPIFQGHVTISADTSINTVYLQWSSLKASDTAMYYCAFRGGVYWGQGTTVTVSS |

CAR5_VH

| | | |
|---|---|---|
| SEQ ID NO: 537 | HCDR1 | GFNIEDYY |
| (IMGT) | | |
| SEQ ID NO: 538 | HCDR2 | IDPENDET |
| (IMGT) | | |
| SEQ ID NO: 539 | HCDR3 | AFRGGVY |
| (IMGT) | | |
| SEQ ID NO: 123 | VH | EIQLVQSGAEVKKPGATVKISCKGSGFNIEDYYIHWVQQAPGKGLEWMGRIDPENDET |
| | | KYGPIFQGRVTITADTSTNTVYMELSSLRSEDTAVYYCAFRGGVYWGQGTTVTVSS |

CAR5_VL

| | | |
|---|---|---|
| SEQ ID NO: 540 | LCDR1 | QSLLDSDGKTY |
| (IMGT) | | |
| SEQ ID NO: 35 | LCDR2 | LVS |
| (IMGT) | | |
| SEQ ID NO: 28 | LCDR3 | WQGTHFPGT |
| (IMGT) | | |
| SEQ ID NO: 126 | VL | DVVMTQSPLSLPVTLGQPASISCKSSQSLLDSDGKTYLNWLQQRPGQSPRRLISLVSK |
| | | LDSGVPDRFSGSGSGTDFTLKISRVEAEDVGVYYCWQGTHFPGTFGGGTKVEIK |

CAR6_VH

| | | |
|---|---|---|
| SEQ ID NO: 537 | HCDR1 | GFNIEDYY |
| (IMGT) | | |
| SEQ ID NO: 538 | HCDR2 | IDPENDET |
| (IMGT) | | |
| SEQ ID NO: 539 | HCDR3 | AFRGGVY |
| (IMGT) | | |
| SEQ ID NO: 124 | VH | EIQLVQSGAEVKKPGESLRISCKGSGFNIEDYYIHWVRQMPGKGLEWMGRIDPENDET |
| | | KYGPIFQGHVTISADTSINTVYLQWSSLKASDTAMYYCAFRGGVYWGQGTTVTVSS |

CAR6_VL

| | | |
|---|---|---|
| SEQ ID NO: 540 | LCDR1 | QSLLDSDGKTY |
| (IMGT) | | |
| SEQ ID NO: 35 | LCDR2 | LVS |
| (IMGT) | | |
| SEQ ID NO: 28 | LCDR3 | WQGTHFPGT |
| (IMGT) | | |
| SEQ ID NO: 127 | VL | DVVMTQSPDSLAVSLGERATINCKSSQSLLDSDGKTYLNWLQQKPGQPPKRLISLVSK |
| | | LDSGVPDRFSGSGSGTDFTLTISSLQAEDVAVYYCWQGTHFPGTFGGGTKVEIK |

CAR7_VL

| | | |
|---|---|---|
| SEQ ID NO: 540 | LCDR1 | QSLLDSDGKTY |
| (IMGT) | | |
| SEQ ID NO: 35 | LCDR2 | LVS |
| (IMGT) | | |
| SEQ ID NO: 28 | LCDR3 | WQGTHFPGT |
| (IMGT) | | |
| SEQ ID NO: 127 | VL | DVVMTQSPDSLAVSLGERATINCKSSQSLLDSDGKTYLNWLQQKPGQPPKRLISLVSK |
| | | LDSGVPDRFSGSGSGTDFTLTISSLQAEDVAVYYCWQGHTFPGTFGGGTKVEIK |

CAR7_VH

| | | |
|---|---|---|
| SEQ ID NO: 537 | HCDR1 | GFNIEDYY |
| (IMGT) | | |
| SEQ ID NO: 538 | HCDR2 | IDPENDET |
| (IMGT) | | |
| SEQ ID NO: 539 | HCDR3 | AFRGGVY |
| (IMGT) | | |
| SEQ ID NO: 124 | VH | EIQLVQSGAEVKKPGESLRISCKGSGFNEIDYYIHWVRQMPGKGLEWMGRIDPENDET |
| | | KYGPIFQGHVTISADTSINTVYLQWSSLKASDTAMYYCAFRGGVYWGQGTTVTVSS |

CAR8_VL

| | | |
|---|---|---|
| SEQ ID NO: 540 | LCDR1 | QSLLDSDGKTY |
| (IMGT) | | |
| SEQ ID NO: 35 | LCDR2 | LVS |
| (IMGT) | | |
| SEQ ID NO: 28 | LCDR3 | WQGTHFPGT |
| (IMGT) | | |
| SEQ ID NO: 126 | VL | DVVMTQSPLSLPVTLGQPASISCKSSQSLLDSDGKTYLNWLQQRPGQSPRRLISLVSK |
| | | LDSGVPDRFSGSGSGTFTLKISRVEAEDVGVYYCWQGTHFPGTFGGGTKVEIK |

CAR8_VH

| | | |
|---|---|---|
| SEQ ID NO: 537 | HCDR1 | GFNIEDYY |
| (IMGT) | | |
| SEQ ID NO: 538 | HCDR2 | IDPENDET |
| (IMGT) | | |
| SEQ ID NO: 539 | HCDR3 | AFRGGVY |
| (IMGT) | | |
| SEQ ID NO: 123 | VH | EIQLVQSGAEVKKPGATVKISCKGSGFNIEDYYIHWVQQAPGKGLEWMGRIDPENDET |
| | | KYGPIFQGRVTITADTSTNTVYMELSSLRSEDTAVYYCAFRGGVYWGQGTTVTVSS |

CAR9_VH_Mouse_anti-EGFRvIII_clone_3C10

| | | |
|---|---|---|
| SEQ ID NO: 537 | HCDR1 | GFNIEDYY |
| (IMGT) | | |
| SEQ ID NO:538 | HCDR2 | IDPENDET |
| (IMGT) | | |
| SEQ ID NO: 539 | HCDR3 | AFRGGVY |
| (IMGT) | | |
| SEQ ID NO: 21 | VH | EIQLQQSGAELVKPGASVKLSCTGSGFNIEDYYIHWVKQRTEQGLEWIGRIDPENDET |
| | | KYGPIFQGRATITADTSSNTVYLQLSSLTSEDTAVYYCAFRGGVYWGPGTTLVTVSS |

CAR9_VL_Mouse_anti-EGFRvIII_clone_3C10

| | | |
|---|---|---|
| SEQ ID NO: 540 | LCDR1 | QSLLDSDGKTY |
| (IMGT) | | |
| SEQ ID NO: 35 | LCDR2 | LVS |
| (IMGT) | | |
| SEQ ID NO: 28 | LCDR3 | WQGTHFPGT |
| (IMGT) | | |
| SEQ ID NO: 25 | VL | DVVMTQSPLTLSVAIGQSASISCKSSQSLLDSDGKTYLNWLLQRPGQSPKRLISLVSK |
| | | LDSGVPDRFTGSGSGTDFTLRISRVEAEDLGIYYCWQGTHFPGTFGGGTKLEIK |

CAR10_VL_Anti-EGFRvIII_clone_139

| | | |
|---|---|---|
| SEQ ID NO: 541 | LCDR1 | QGIRNN |
| (IMGT) | | |
| SEQ ID NO: 542 | LCDR2 | AAS |
| (IMGT) | | |
| SEQ ID NO: 543 | LCDR3 | LQHHSYPLT |
| (IMGT) | | |
| SEQ ID NO: 544 | VL | DIQMTQSPSSLSASVGDRVTITCRASQGIRNNLAWYQQKPGKAPKRLIYAASNLQSGV |
| | | PSRFTGSGSGTEFTLIVSSLQPEDFATYYCLQHHSYPLTSGGGTKVEIK |

CAR10_VH_Anti-EGFRvIII_clone_139

| | | |
|---|---|---|
| SEQ ID NO: 545 | HCDR1 | GFTFSSYA |
| (IMGT) | | |
| SEQ ID NO: 546 | HCDR2 | ISGSGGST |
| (IMGT) | | |
| SEQ ID NO: 547 | HCDR3 | AGSSGWSEY |
| (IMGT) | | |
| SEQ ID NO: 548 | VH | EVQLVESGGGLVQPGGSLRLSCAASGFTFSSYAMSWVRQAPGKGLEWVSAISGSGGST |
| | | NYADSVKGRFTISRDNSKNTLYLQMNSLRAEDTAVYYCAGSSGWSEYWGQGTLVTVSS |

The CAR scFv fragments were then cloned into lentiviral vectors to create a full length CAR construct in a single coding frame, and using the EF1 alpha promoter for expression (SEQ ID NO: 97).

EF1 alpha promoter
(SEQ ID NO: 97)
GTGAGGCTCCGGTGCCCGTCAGTGGGCAGAGCGCACATCGCCCACAGTCC
CCGAGAAGTTGGGGGGAGGGGTCGGCAATTGAACCGGTGCCTAGAGAAGG
TGGCGCGGGGTAAACTGGGAAAGTGATGTCGTGTACTGGCTCCGCCTTTT
TCCCGAGGGTGGGGGAGAACCGTATATAAGTGCAGTAGTCGCCGTGAACG
TTCTTTTTCGCAACGGGTTTGCCGCCAGAACACAGGTAAGTGCCGTGTGT
GGTTCCCGCGGGCCTGGCCTCTTTACGGGTTATGGCCCTTGCGTGCCTTG
AATTACTTCCACCTGGCTGCAGTACGTGATTCTTGATCCCGAGCTTCGGG
TTGGAAGTGGGTGGGAGAGTTCGAGGCCTTGCGCTTAAGGAGCCCCTTCG
CCTCGTGCTTGAGTTGAGGCCTGGCCTGGGCGCTGGGGCCGCCGCGTGCG
AATCTGGTGGCACCTTCGCGCCTGTCTCGCTGCTTTCGATAAGTCTCTAG
CCATTTAAAATTTTTGATGACCTGCTGCGACGCTTTTTTTCTGGCAAGAT
AGTCTTGTAAATGCGGGCCAAGATCTGCACACTGGTATTTCGGTTTTTGG
GGCCGCGGGCGGCGACGGGGCCCGTGCGTCCCAGCGCACATGTTCGGCGA
GGCGGGGCCTGCGAGCGCGGCCACCGAGAATCGGACGGGGGTAGTCTCAA
GCTGGCCGGCCTGCTCTGGTGCCTGGCCTCGCGCCGCCGTGTATCGCCCC
GCCCTGGGCGGCAAGGCTGGCCCGGTCGGCACCAGTTGCGTGAGCGGAAA
GATGGCCGCTTCCCGGCCCTGCTGCAGGGAGCTCAAAATGGAGGACGCGG
CGCTCGGGAGAGCGGGCGGGTGAGTCACCCACACAAAGGAAAAGGGCCTT
TCCGTCCTCAGCCGTCGCTTCATGTGACTCCACGGAGTACCGGGCGCCGT
CCAGGCACCTCGATTAGTTCTCGAGCTTTTGGAGTACGTCGTCTTTAGGT
TGGGGGGAGGGGTTTTATGCGATGGAGTTTCCCCACACTGAGTGGGTGGA
GACTGAAGTTAGGCCAGCTTGGCACTTGATGTAATTCTCCTTGGAATTTG
CCCTTTTTGAGTTTGGATCTTGGTTCATTCTCAAGCCTCAGACAGTGGTT
CAAAGTTTTTTTCTTCCATTTCAGGTGTCGTGA.

### Surface Expression of CAR9, CAR10 and Select Humanized EGFRvIII CAR Constructs and Staining by FACS

The following experiments showed that there appears to be an affinity difference for EGFRvIII based in vitro binding studies in both Jurkat cells and primary T cells.

Jurkat E6 cells were electroporated with either CAR9 vector or CAR10 vector using Amaxa Cell Line Nucleofector Kit V (Lonza, Colgne AG, Germany) and program X-001. One day after the transfection, 0.5×10⁶ cells were placed into each well of a V-shape 96 well plate (Greiner Bio-One, Germany) in 0.2 ml FACS buffer (DPBS buffer containing 5% FBS) and incubated for 10 minutes at room temperature. Cells were then spun down and resuspended in 0.2 ml of the FACS buffer with different concentrations of EGFRvIII-Fc or EGFRwt-Fc and incubated at 4° C. for 30 minutes. Cells were then washed with FACS buffer three times, and incubated with 0.2 ml of the FACS buffer with 2 μl of PE anti-human IgG Fc (Jackson ImmunoResearch Laboratories, West Grove, Pa.) for 30 minutes at 4° C. in the dark. After washing with 0.2 ml of FACS buffer three times, cells were analyzed on a LSRII (BD Biosciences, San Jose, Calif.) machine using the FACSDiva software (BD Biosciences, San Jose, Calif.). Immunofluorescence staining was analyzed as the relative log fluorescence of live cells, and the percentage of the PE positive cells were measured.

As shown in FIG. 12 of US2014/0322275A1, binding of the CAR9 expressed in Jurkat cells to EGFRvIII-Fc fusion protein is approximately 1000 fold stronger than to wild type EGFR-Fc. Furthermore, the CART construct expressing CAR10 exhibits a significantly lower (˜40 fold) binding to EGFRvIII compared to CAR9. This suggests that although murine CAR9 binds to EGFRvIII, it still retains some binding to wild type EGFR. Moreover, it strongly indicates that CAR9 has a higher binding affinity for EGFRvIII than the CAR10 construct.

Further experiments in primary T cells yielded similar results. Briefly, primary human CD3+ T cells were stimulated with anti-CD3/CD28 beads for 24 hrs and then transduced with lentiviral vectors encoding either CAR9, CAR10, CAR6 or a control CAR at a MOI of 3:1. Included in the experiment was also a mock transduced T cell population. These cells were expanded for about 8-9 days in culture until they started to rest down. At this point, 0.5×106 cells were placed into each well of a V-shape 96 well plate. The cells were washed one time with PBS and stained with Live/Dead reagent (1:1000 in PBS) for 30 min on ice. Cells were then washed twice with FACS buffer and incubated with 1 □g/ml biotinylated EGFRvIII or EGFR wt protein for 30 min on ice. Cells were then washed two times and incubated with 0.2 ml of FACS buffer with 1:1000 dilution of streptavidin-PE for 15 min on ice. After washing twice with FACS buffer, cells were analyzed on a LSRII. Immunofluorescence staining was analyzed as the relative log fluorescence of live cells and the percentage of the PE positive cells were measured in conjunction with the geometric mean of the positive population.

As shown in FIG. 13 of US2014/0322275A1, the CAR9 and CAR6 CARs show a 10-fold higher geometric mean (21K for CAR9, 27K for CAR6) for EGFRvIII binding than the CAR10 (only 2K) when saturating amounts of EGFRvIII protein are used for detection, even though all constructs transduce equivalently (˜50% transduction efficiency for all). Similarly, the specificity for EGFR wt protein is about 10-fold lower, as depicted by the log shift downwards for the staining with EGFR wt protein. This provides additional support to the findings in the Jurkat cells above that indicate CAR9 and CAR6 have a stronger affinity for EGFRvIII protein compared to CAR10 when expressed in primary T cells and suggest they will be more efficacious in the clinic.

Functional analysis of the panel of humanized CAR constructs was conducted as described in Example 8 of US2014/0322275A1.

### Example 2: Mouse Model of EGFRvIII CAR T Cells and PD-1

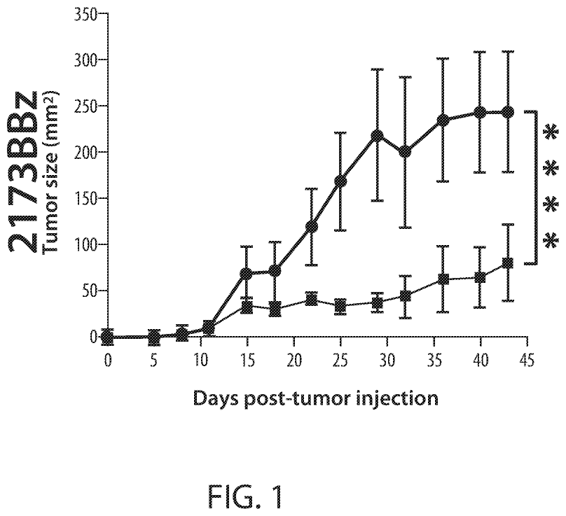

A patient-derived xenograft glioma line, D270, was used to generate a model system in NGS mice. It as previously been shown that D270 expresses high levels of EGFRvIII and demonstrates a pathology similar to human gliomas in vivo. Subcutaneous tumors were implanted in the mice. After tumor implantation, the mice were treated with both EGFRvIII CAR T cells (expressing 2173 EGFRvIII CAR) and PD-1 blockade. While PD-1 alone demonstrated no efficacy, the combination of EGFvIII and PD-1 resulted in synergistic grown inhibition (FIG. 1).

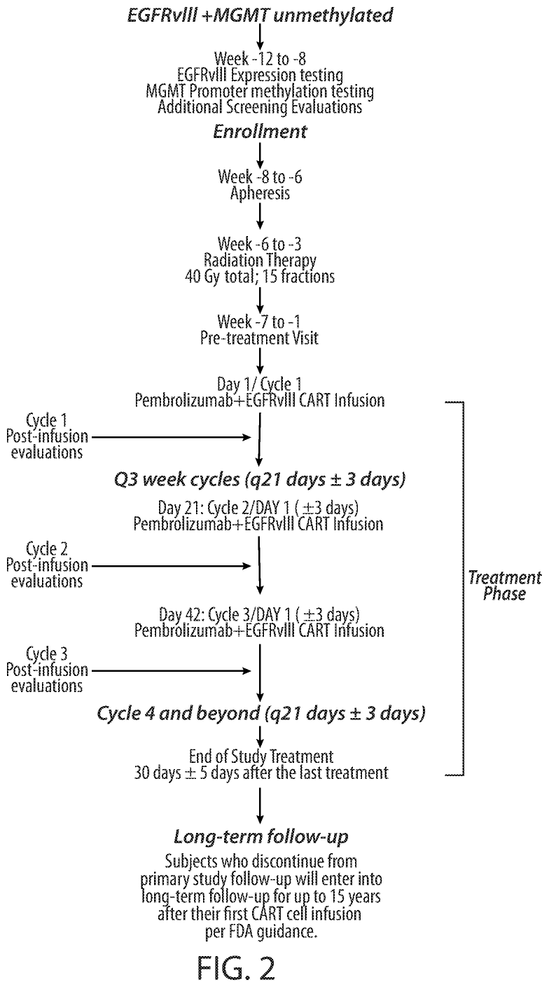

### Example 3: EGFRvIII-Directed CAR T Cells Combined with PD-1 Inhibition in Patients with MGMT-Unmethylated Glioblastoma (See Exemplary Schematic in FIG. 2)

Adult subjects (18 and over) are identified, each having newly diagnosed glioblastoma (GBM) that has not been previously been treated with a systemic therapy. The following inclusion/exclusion criteria are applied:

### Inclusion Criteria:

1. Newly diagnosed glioblastoma (GBM) that is histologically confirmed by pathology review of surgically resected tissue.
2. Undergone tumor resection.
3. No prior systemic therapies, radiation, tumor-treating fields, or intratumoral therapeutic agents including Gliadel wafers are allowed. Tumor resection must be the only tumor-directed treatment that the patient has received for glioboblastoma.
4. Tumor tissue is positive for EGFRvIII expression, as performed by the University of Pennsylvania's in-house fusion transcript panel (RNA-based assay using Illumina HiSeq platform). Outside EGFRvIII expression testing is not allowed.
5. Tumor tissue is negative for MGMT promoter methylation (i.e. the tumor is MGMT-unmethylated), as performed by the University of Pennsylvania's in-house pyrosequencing protocol. Outside MGMT testing is not allowed.
6. Patients ≥18 years of age
7. ECOG performance status 0-1
8. Provides written informed consent
9. Must have adequate organ function as measured by:
   1. White blood count ≥2500/mm3; platelets ≥100,000/mm3, hemoglobin ≥9.0 g/dL; without transfusion or growth factor support
   2. AST, ALT, GGT, LDH, alkaline phosphatase within 2.5×upper normal limit, and total bilirubin ≤2.0 mg/dL
   3. Serum creatinine <1.5×upper limit of normal
   4. Adequate cardiac function (LVEF ≥45%)
10. Subjects of reproductive potential must agree to use acceptable birth control methods.

Exclusion Criteria:
1. Pregnant or lactating women
2. Inadequate venous access for or contraindications to leukapheresis.
3. Active Hepatitis B, hepatitis C, or HIV infection, or other active, uncontrolled infection
4. History of allergy or hypersensitivity to study product excipients (human serum albumin, DMSO, and Dextran 40)
5. History of severe hypersensitivity reactions to other monoclonal antibodies which in the opinion of the investigator may post an increased risk of serious infusion reactions.
6. Requirement for immunosuppressive agents including but not limited to cyclosporine, MMF, tacrolimus, rapamycin, or anti-TNF agents within 4 weeks of eligibility confirmation by the physician-investigator.
7. Subjects with a history of known or suspected, severe or uncontrolled autoimmune or connective tissue disease. Patients with vitiligo, controlled type 1 diabetes mellitus (on stable insulin dose), residual autoimmune-related hypothyroidism (due to autoimmune condition only requiring hormone replacement), or psoriasis (not requiring systemic treatment), or conditions not expected to recur in the absence of an external trigger, are permitted to enroll.
8. Known history or current interstitial lung disease or non-infectious pneumonitis
9. Prior allogenic bone marrow or solid organ transplant
10. Any concurrent malignancy other than non-melanoma skin cancer that has been curatively treated, or carcinoma in situ of the cervix or bladder that has been curatively treated. For any prior invasive malignancy, at least 5 years must have elapsed since curative therapy and patients must not have received any radiation to the brain. Monoclonal gammopathy of undetermined significance (MGUS) is permitted.
11. Any uncontrolled active medical or psychiatric disorder that would preclude participation as outlined.
12. Severe, active co-morbidity in the opinion of the physician-investigator would preclude participation in this study, including but not limited to the following:
   1. Unstable angina within 6 months prior to eligibility confirmation by the physician-investigator
   2. Transmural myocardial infarction within the last 6 months prior to eligibility confirmation by the physician-investigator
   3. New York Heart Association grade II or greater congestive heart failure requiring hospitalization within 12 months prior to eligibility confirmation by the physician-investigator.
   4. Serious and inadequately controlled cardiac arrhythmia
   5. Serious or non-healing wound, ulcer, or history of abdominal fistula, gastrointestinal perforation, intra-abdominal abscess major surgical procedure, open biopsy, or significant traumatic injury within 28 days prior to eligibility confirmation by the physician-investigator, with the exception of the craniotomy for tumor resection.

The subjects' tumors are assessed for EGFRvIII expression by an RNA-based assay. Subjects' tumors are further screened to identify those subjects with tumors negative for MGMT promoter methylation (i.e., the tumor is MGMT-unmethylated) by pyrosequencing. Subjects are further assessed for other qualifying and disqualifying criteria, e.g. reproductive status and/or the presence of other malignancies or active medical or psychiatric disorder. Subjects that have MGMT-unmethylated tumors that have undergone either complete or partial resection and satisfy the remaining criteria are selected for the study. Subjects further must be taking at least one anti-epileptic medication at the time of enrollment, e.g. 24 hours prior to the initiation of the study treatment, and take the drug throughout the course of the study.

For each patient, large volume aphresis is performed with the intention to harvest at least 5×10⁹ white blood cells to manufacture CAR T cells. Additional aphresis may be carried out to yield an adewuate number of cells. EGFRvIII CAR T cells are generated from these cells

No more than six weeks after surgery, a short course of radiation—with a total does of 40 Gy delivered in 15 fractions over 3 weeks—is administered to the subjects.

Prior to receiving the study treatment, subjects are screened to establish a baseline, e.g., current medical conditions, a physical, standard neurological tests, vitals, ECO performance, concomitant medications, complete blood count and differential, chemistry panels, serum pregnancy (females, only), HLH/MAS, coagulation factors, endocrine evaluation, EKG, and MRI. Subjects that still qualify receive an infusion of about 1×10⁸-2×10⁸ of the EGFRvIII CAR T cells (via rapid infusion) and 200 mg of pembrolizumab (IV) every three weeks. Subjects may receive up to 4 infusions of pembrolizumab so longs as it provides a clinical benefit and 3 infusions of the EGFRvIII CAR T cells depending on availability.

Subjects are monitored to determine safety and tolerability. Overall survival, disease progression (determined by MRI and RANO criteria), and objective response rate (determined by modified RANO criteria) are further assessed. The combination treatment of EGFRvIII CAR T cells and anti-PD-1 is found to have a synergistic effect on treatment of MGMT-unmethylated GBM.

### EQUIVALENTS

The disclosures of each and every patent, patent application, and publication cited herein are hereby incorporated herein by reference in their entirety. While this invention has been disclosed with reference to specific aspects, it is apparent that other aspects and variations of this invention may be devised by others skilled in the art without departing from the true spirit and scope of the invention. The appended claims are intended to be construed to include all such aspects and equivalent variations.

What is claimed is:

1. A CAR therapy comprising a population of immune effector cells expressing a chimeric antigen receptor (CAR) for use in combination with a PD-1 inhibitor, wherein the CAR comprises an antigen (e.g., a EGFRvIII) binding domain, a transmembrane domain and an intracellular signaling domain, and wherein the dose of the PD-1 inhibitor, e.g., anti-PD-1 antibody molecule, is about 200 mg to about 450 mg, e.g., about 300 mg to about 400 mg, e.g., administered every 2 weeks, 3 weeks, 4 weeks, or 5 weeks.

2. A method of treating a subject having a cancer, comprising administering to the subject:
(i) a CAR therapy comprising a population of immune effector cells expressing a chimeric antigen receptor (CAR), wherein the CAR comprises an antigen (e.g., a EGFRvIII) binding domain, a transmembrane domain, and an intracellular signaling domain; and
(ii) a PD-1 inhibitor,
wherein the dose of the PD-1 inhibitor, e.g., anti-PD-1 antibody molecule, is about 200 mg to about 450 mg, e.g., about 300 mg to about 400 mg, e.g., administered every 2 weeks, 3 weeks, 4 weeks, or 5 weeks.

3. A CAR therapy comprising a population of immune effector cells expressing a chimeric antigen receptor (CAR) for use in combination with a PD-1 inhibitor, wherein the CAR comprises an antigen (e.g., a EGFRvIII) binding domain, a transmembrane domain and an intracellular signaling domain, and wherein administration of the PD-1 inhibitor is initiated 20 days or less after administration of the CAR therapy.

4. A method of treating a subject having a cancer, comprising administering to the subject:
(i) a CAR therapy comprising a population of immune effector cells expressing a chimeric antigen receptor (CAR), wherein the CAR comprises an antigen (e.g., EGFRvIII) binding domain, a transmembrane domain, and an intracellular signaling domain; and
(ii) a PD-1 inhibitor,
wherein administration of the PD-1 inhibitor is initiated 20 days or less after administration of the CAR therapy.

5. The CAR therapy for use or the method of claim 3 or 4, wherein administration of the PD-1 inhibitor is initiated 16 days or less, 15 days or less, 14 days or less, 13 days or less, 12 days or less, 11 days or less, 10 days or less, 9 days or less, 8 days or less, 7 days or less, 6 days or less, 5 days or less, 4 days or less, 3 days or less, 2 days or less, after administration of the CAR therapy.

6. A CAR therapy comprising a population of immune effector cells expressing a chimeric antigen receptor (CAR) for use in combination with a PD-1 inhibitor, wherein the CAR comprises an antigen (e.g., a EGFRvIII) binding domain, a transmembrane domain and an intracellular signaling domain, and wherein administration of the PD-1 inhibitor is initiated after the subject has, or is identified as having, one or more of the following:
(a) a partial or no detectable response to the CAR therapy,
(b) a relapsed cancer after the CAR therapy,
(c) a cancer refractory to the CAR therapy; or
(d) a progressive form of the cancer after the CAR therapy.

7. A method of treating a subject having a cancer, comprising administering to the subject:
(i) a CAR therapy comprising a population of immune effector cells expressing a chimeric antigen receptor (CAR), wherein the CAR comprises an antigen (e.g., a EGFRvIII) binding domain, a transmembrane domain, and an intracellular signaling domain; and
(ii) a PD-1 inhibitor,
wherein administration of the PD-1 inhibitor is initiated after the subject has, or is identified as having, one or more of the following:
(a) a partial or no detectable response to the CAR therapy,
(b) a relapsed cancer after the CAR therapy,
(c) a cancer refractory to the CAR therapy; or
(d) a progressive form of the cancer after the CAR therapy.

8. A CAR therapy comprising a population of immune effector cells expressing a chimeric antigen receptor (CAR) for use in combination with a PD-1 inhibitor, wherein the CAR comprises an antigen (e.g., a EGFRvIII) binding domain, a transmembrane domain and an intracellular signaling domain, and wherein administration of the PD-1 inhibitor is initiated after administration of the CAR therapy, and the subject does not have, or has not been identified as having, one or more of the following:
(a) a partial or no detectable response to the CAR therapy,
(b) a relapsed cancer after the CAR therapy,
(c) a cancer refractory to the CAR therapy, or
(d) a progressive form of the cancer.

9. A method of treating a subject having a cancer, comprising administering to the subject:
(i) a CAR therapy comprising a population of immune effector cells expressing a chimeric antigen receptor (CAR), wherein the CAR comprises an antigen (e.g., a EGFRvIII) binding domain, a transmembrane domain, and an intracellular signaling domain; and
(ii) a PD-1 inhibitor,
wherein administration of the PD-1 inhibitor is initiated after administration of the CAR therapy, and the subject does not have, or has not been identified as having, one or more of the following:
(a) a partial or no detectable response to the CAR therapy,
(b) a relapsed cancer after the CAR therapy,
(c) a cancer refractory to the CAR therapy, or
(d) a progressive form of the cancer.

10. The CAR therapy for use or the method of any of the preceding claims, further comprising administering one or more, e.g., 1, 2, 3, 4, or more, subsequent doses of the PD-1 inhibitor.

11. The CAR therapy for use or the method of claim 10, wherein up to 4 doses of the PD-1 inhibitor are administered.

12. The CAR therapy for use or the method of any of claims 1-11, wherein the method further comprising evaluating the presence or absence of CRS in the subject.

13. The CAR therapy for use or the method of any of claims 1-12, wherein the subject does not have, or is identified, as not having CRS, e.g., severe CRS (e.g., CRS grade 3 or grade 4), after the CAR therapy.

14. The CAR therapy for use or the method of either of claim 12 or 13, wherein administration of the PD-1 inhibitor is initiated after the subject is identified as not having CRS, e.g., severe CRS (e.g., CRS grade 3 or grade 4), after the CAR therapy.

15. The CAR therapy for use or the method of any of claims 12-14, wherein administration of the PD-1 inhibitor is initiated after treatment of CRS, e.g., after CRS resolution, after the CAR therapy.

16. The CAR therapy for use or the method of any of the preceding claims, wherein the CAR therapy and the PD-1 inhibitor are administered for a treatment interval, and wherein the treatment interval comprises a single dose of the PD-1 inhibitor and a single dose of the CAR-expressing cell.

17. The CAR therapy for use or the method of claim 16, wherein the treatment interval is initiated upon administration of the dose of the CAR-therapy and completed upon administration of the dose of the PD-1 inhibitor.

18. The CAR therapy for use or the method of claim 16 or 17, wherein the treatment interval further comprises administering one or more, e.g., 1, 2, 3, 4, or more, subsequent doses of the PD-1 inhibitor.

19. The CAR therapy for use or the method of claim 18, wherein up to 4 doses of the PD-1 inhibitor are administered during the treatment interval.

20. The CAR therapy for use or the method of any of claims 1-19, wherein the dose of the CAR-therapy is administered concurrently with the dose of PD-1 inhibitor is administered.

21. The CAR therapy for use or the method of any of claims 1-20, wherein the treatment interval further comprises administering one or more, e.g., 1, 2, 3, or more, subsequent doses of the population of immune effector cells expressing a chimeric antigen receptor (CAR).

22. The CAR therapy for use or the method of claim 21, wherein up to 3 doses of the population of immune effector cells expressing a chimeric antigen receptor (CAR) are administered during the treatment interval.

23. The CAR therapy for use or the method of any of claims 1-22, wherein the treatment interval is repeated, e.g., one or more times, e.g., 1, 2, 3, 4, or more times.

24. The CAR therapy for use or the method of claim 23, wherein the one or more subsequent treatment intervals is administered at least 3 weeks after the completion of the first or previous treatment interval.

25. The CAR therapy for use or the method of any of claims 1-24, wherein the CAR-therapy comprises an RNA CAR molecule, e.g., an in vitro transcribed (IVT) RNA, and wherein one or more, e.g., 1, 2, 3, 4, or 5, subsequent doses of a CAR-therapy is administered to the subject after the initial dose of the CAR-therapy.

26. The CAR therapy for use or the method of any of the preceding claims, wherein the CAR therapy comprises a dose of CAR-expressing cells comprising about 1.75-5×10⁸ cells.

27. The CAR therapy for use or the method claim 26, wherein the dose of CAR-expressing cells is about 2×10⁸ cells.

28. The CAR therapy for use or the method of claim 26, wherein the dose of CAR-expressing cells is about 5×10⁸ cells.

29. The CAR therapy for use or the method of any of claims 1-28, wherein the dose of the PD-1 inhibitor is about 200 mg or about 300 mg, e.g., administered every 3 weeks, e.g., via intravenous infusion.

30. The CAR therapy for use or the method of any of claims 1-29, wherein the PD-1 inhibitor is a PD-1 antibody molecule and is administered at a dose of about 200 mg every 2 weeks, 3 weeks, or 4 weeks, and the CAR therapy is administered at a dose of 1.75-5×10⁸ cells.

31. The CAR therapy for use or the method of any of the preceding claims, wherein the PD-1 inhibitor comprises an antibody molecule, a small molecule, a polypeptide, e.g., a fusion protein, or an inhibitory nucleic acid, e.g., a siRNA or shRNA.

32. The CAR therapy for use or the method of any of the preceding claims, wherein the PD-1 inhibitor is characterized by one or more of the following:
a. inhibits or reduces PD-1 expression, e.g., transcription or translation of PD-1;
b. inhibits or reduces PD-1 activity, e.g., inhibits or reduces binding of PD-1 to its cognate ligand, e.g., PD-L1 or PD-L2; or
c. binds to PD-1 or its ligand(s), e.g., PD-L1 or PD-L2.

33. The CAR therapy for use or the method of any of the preceding claims, wherein the PD-1 inhibitor is an antibody molecule.

34. The CAR therapy for use or the method of any of the preceding claims, wherein the PD-1 inhibitor is selected from the group consisting of Nivolumab, Pembrolizumab, PDR001, Pidilizumab, AMP 514, AMP-224, and any anti-PD-1 antibody molecule provided in Table 6.

35. The CAR therapy for use or the method of any of the preceding claims, wherein the PD-1 inhibitor comprises an anti-PD-1 antibody molecule comprising
d. a heavy chain complementary determining region 1 (HC CDR1), a heavy chain complementary determining region 2 (HC CDR2), and a heavy chain complementary determining region 3 (HC CDR3) of any PD-1 antibody molecule amino acid sequence listed in Table 6; and
e. a light chain complementary determining region 1 (LC CDR1), a light chain complementary determining region 2 (LC CDR2), and a light chain complementary determining region 3 (LC CDR3) of any PD-1 antibody molecule amino acid sequence listed in Table 6.

36. The CAR therapy for use or the method of claim 35 wherein the anti-PD-1 antibody molecule thereof comprises a) a HC CDR1 amino acid sequence chosen from SEQ ID NO: 137 or 140, a HC CDR2 amino acid sequence of SEQ ID NO: 138 or 141, and a HC CDR3 amino acid sequence of SEQ ID NO: 139; and
b) a LC CDR1 amino acid sequence of SEQ ID NO: 146 or 149, a LC CDR2 amino acid sequence of SEQ ID NO: 147 or 150, and a LC CDR3 amino acid sequence of SEQ ID NO: 148, 151, 166, or 167 (e.g., a LC CDR3 amino acid sequence of SEQ ID NO: 166 or 167).

37. The CAR therapy for use or the method of claim 35 or 36, wherein the anti-PD-1 antibody molecule comprises a heavy chain variable region comprising:
i) the amino acid sequence of any heavy chain variable region listed in Table 6, e.g., SEQ ID NOs: 142, 144, 154, 158, 172, 184, 216, or 220;
ii) the amino acid sequence having at least one, two, or three modifications but not more than 30, 20, or 10 modifications to the amino acid sequence of any heavy chain variable region provided in Table 6, e.g., SEQ ID NOs: 142, 144, 154, 158, 172, 184, 216, or 220; or
iii) an amino acid sequence with 95-99% identity to the amino acid sequence of any heavy chain variable region provided in Table 6, e.g., SEQ ID NOs: 142, 144, 154, 158, 172, 184, 216, or 220.

38. The CAR therapy for use or the method of any of claims 35-37, wherein the anti-PD-1 antibody molecule comprises a heavy chain comprising:
i) the amino acid sequence of any heavy chain listed in Table 6, e.g., SEQ ID NOs: 156, 160, 174, 186, 218, 222, 225, or 236;
ii) the amino acid sequence having at least one, two, or three modifications but not more than 30, 20, or 10 modifications to any heavy chain listed in Table 6, e.g., SEQ ID NOs: 156, 160, 174, 186, 218, 222, 225, or 236; or
iii) an amino acid sequence with 95-99% identity to the amino acid sequence of any heavy chain listed in Table 6, e.g., SEQ ID NOs: 156, 160, 174, 186, 218, 222, 225, or 236.

39. The CAR therapy for use or the method of any of claims 35-38, wherein the anti-PD-1 antibody molecule comprises a light chain variable region comprising:
i) the amino acid sequence of any light chain variable region listed in Table 6, e.g., SEQ ID NOs: 152, 162, 168, 176, 180, 188, 192, 196, 200, 204, 208, or 212;
ii) the amino acid sequence having at least one, two, or three modifications but not more than 30, 20, or 10 modifications to the amino acid sequence of any light chain variable region provided in Table 6, e.g., SEQ ID NOs: 152, 162, 168, 176, 180, 188, 192, 196, 200, 204, 208, or 212; or
iii) an amino acid sequence with 95-99% identity to the amino acid sequence of any light chain variable region provided in Table 6, e.g., SEQ ID NOs: 152, 162, 168, 176, 180, 188, 192, 196, 200, 204, 208, or 212.

40. The CAR therapy for use or the method of any of claims 35-39, wherein the anti-PD-1 antibody molecule comprises a light chain comprising:
i) the amino acid sequence of any light chain listed in Table 6, e.g., SEQ ID NOs: 164, 170, 178, 182, 190, 194, 198, 202, 206, 210, or 214;
ii) the amino acid sequence having at least one, two, or three modifications but not more than 30, 20, or 10 modifications to any light chain listed in Table 6, e.g., SEQ ID NOs: 164, 170, 178, 182, 190, 194, 198, 202, 206, 210, or 214; or
iii) an amino acid sequence with 95-99% identity to the amino acid sequence to any any light chain listed in Table 6, e.g., SEQ ID NOs: 164, 170, 178, 182, 190, 194, 198, 202, 206, 210, or 214.

41. The CAR therapy for use or the method of any of claims 35-40, wherein the anti-PD-1 antibody molecule comprises:
i) a heavy chain variable domain comprising the amino acid sequence of SEQ ID NO: 172 and a light chain variable domain comprising the amino acid sequence of SEQ ID NO: 204 ii) a heavy chain variable domain comprising the amino acid sequence of SEQ ID NO: 142 or 144 and a light chain variable domain comprising the amino acid sequence of SEQ ID NO: 152;
iii) a heavy chain variable domain comprising the amino acid sequence of SEQ ID NO: 154 or 158 and a light chain variable domain comprising the amino acid sequence of SEQ ID NO: 162;
iv) a heavy chain variable domain comprising the amino acid sequence of SEQ ID NO: 154 or 158 and a light chain variable domain comprising the amino acid sequence of SEQ ID NO: 168;
v) a heavy chain variable domain comprising the amino acid sequence of SEQ ID NO: 172 and a light chain variable domain comprising the amino acid sequence of SEQ ID NO: 176;
vi) a heavy chain variable domain comprising the amino acid sequence of SEQ ID NO: 172 and a light chain variable domain comprising the amino acid sequence of SEQ ID NO: 180;
vii) a heavy chain variable domain comprising the amino acid sequence of SEQ ID NO: 184 and a light chain variable domain comprising the amino acid sequence of SEQ ID NO: 180;
viii) a heavy chain variable domain comprising the amino acid sequence of SEQ ID NO: 184 and a light chain variable domain comprising the amino acid sequence of SEQ ID NO: 188;
ix) a heavy chain variable domain comprising the amino acid sequence of SEQ ID NO: 172 and a light chain variable domain comprising the amino acid sequence of SEQ ID NO: 188;
x) a heavy chain variable domain comprising the amino acid sequence of SEQ ID NO: 172 and a light chain variable domain comprising the amino acid sequence of SEQ ID NO: 192;
xi) a heavy chain variable domain comprising the amino acid sequence of SEQ ID NO: 172 and a light chain variable domain comprising the amino acid sequence of SEQ ID NO: 196;
xii) a heavy chain variable domain comprising the amino acid sequence of SEQ ID NO: 184 and a light chain variable domain comprising the amino acid sequence of SEQ ID NO: 200;
xiii) a heavy chain variable domain comprising the amino acid sequence of SEQ ID NO: 172 and a light chain variable domain comprising the amino acid sequence of SEQ ID NO: 200;
xiv) a heavy chain variable domain comprising the amino acid sequence of SEQ ID NO: 184 and a light chain variable domain comprising the amino acid sequence of SEQ ID NO: 204;
xv) a heavy chain variable domain comprising the amino acid sequence of SEQ ID NO: 172 and a light chain variable domain comprising the amino acid sequence of SEQ ID NO: 204;
xvi) a heavy chain variable domain comprising the amino acid sequence of SEQ ID NO: 172 and a light chain variable domain comprising the amino acid sequence of SEQ ID NO: 208;
xvii) a heavy chain variable domain comprising the amino acid sequence of SEQ ID NO: 172 and a light chain variable domain comprising the amino acid sequence of SEQ ID NO: 212;
xviii) a heavy chain variable domain comprising the amino acid sequence of SEQ ID NO: 216 and a light chain variable domain comprising the amino acid sequence of SEQ ID NO: 204;
xix) a heavy chain variable domain comprising the amino acid sequence of SEQ ID NO: 216 and a light chain variable domain comprising the amino acid sequence of SEQ ID NO: 200;
xx) a heavy chain variable domain comprising the amino acid sequence of SEQ ID NO: 220 and a light chain variable domain comprising the amino acid sequence of SEQ ID NO: 200;
xxi) a heavy chain variable domain comprising the amino acid sequence of SEQ ID NO: 172 and a light chain variable domain comprising the amino acid sequence of SEQ ID NO: 176;
xxii) a heavy chain variable domain comprising the amino acid sequence of SEQ ID NO: 172 and a light chain variable domain comprising the amino acid sequence of SEQ ID NO: 188;
xxiii) a heavy chain variable domain comprising the amino acid sequence of SEQ ID NO: 172 and a light chain variable domain comprising the amino acid sequence of SEQ ID NO: 200; or
xxiv) a heavy chain variable domain comprising the amino acid sequence of SEQ ID NO: 184 and a light chain variable domain comprising the amino acid sequence of SEQ ID NO: 204.

42. The CAR therapy for use or the method of any of claims 35-41, wherein the anti-PD-1 antibody molecule comprises:
a. a heavy chain comprising the amino acid sequence of SEQ ID NO: 225 and a light chain comprising the amino acid sequence of SEQ ID NO: 206;
b. a heavy chain comprising the amino acid sequence of SEQ ID NO: 144 and a light chain comprising the amino acid sequence of SEQ ID NO: 152;
c. a heavy chain comprising the amino acid sequence of SEQ ID NO: 156 or 160 and a light chain comprising the amino acid sequence of SEQ ID NO: 164;
d. a heavy chain comprising the amino acid sequence of SEQ ID NO: 156 or 160 and a light chain comprising the amino acid sequence of SEQ ID NO: 170.
e. a heavy chain comprising the amino acid sequence of SEQ ID NO: 174 and a light chain comprising the amino acid sequence of SEQ ID NO: 178;
f. a heavy chain comprising the amino acid sequence of SEQ ID NO: 174 and a light chain comprising the amino acid sequence of SEQ ID NO: 182;
g. a heavy chain comprising the amino acid sequence of SEQ ID NO: 186 and a light chain comprising the amino acid sequence of SEQ ID NO: 182;
h. a heavy chain comprising the amino acid sequence of SEQ ID NO: 186 and a light chain comprising the amino acid sequence of SEQ ID NO: 190;
i. a heavy chain comprising the amino acid sequence of SEQ ID NO: 174 and a light chain comprising the amino acid sequence of SEQ ID NO: 190;
j. a heavy chain comprising the amino acid sequence of SEQ ID NO: 174 and a light chain comprising the amino acid sequence of SEQ ID NO: 194;
k. a heavy chain comprising the amino acid sequence of SEQ ID NO: 174 and a light chain comprising the amino acid sequence of SEQ ID NO: 198;
l. a heavy chain comprising the amino acid sequence of SEQ ID NO: 186 and a light chain comprising the amino acid sequence of SEQ ID NO: 202;
m. a heavy chain comprising the amino acid sequence of SEQ ID NO: 174 and a light chain comprising the amino acid sequence of SEQ ID NO: 202;
n. a heavy chain comprising the amino acid sequence of SEQ ID NO: 186 and a light chain comprising the amino acid sequence of SEQ ID NO: 206;
o. a heavy chain comprising the amino acid sequence of SEQ ID NO: 174 and a light chain comprising the amino acid sequence of SEQ ID NO: 206;
p. a heavy chain comprising the amino acid sequence of SEQ ID NO: 174 and a light chain comprising the amino acid sequence of SEQ ID NO: 210;
q. a heavy chain comprising the amino acid sequence of SEQ ID NO: 174 and a light chain comprising the amino acid sequence of SEQ ID NO: 214;
r. a heavy chain comprising the amino acid sequence of SEQ ID NO: 218 and a light chain comprising the amino acid sequence of SEQ ID NO: 206;
s. a heavy chain comprising the amino acid sequence of SEQ ID NO: 218 and a light chain comprising the amino acid sequence of SEQ ID NO: 202;
t. a heavy chain comprising the amino acid sequence of SEQ ID NO: 222 and a light chain comprising the amino acid sequence of SEQ ID NO: 202;
u. a heavy chain comprising the amino acid sequence of SEQ ID NO: 225 and a light chain comprising the amino acid sequence of SEQ ID NO: 178;
v. a heavy chain comprising the amino acid sequence of SEQ ID NO: 225 and a light chain comprising the amino acid sequence of SEQ ID NO: 190;
w. a heavy chain comprising the amino acid sequence of SEQ ID NO: 225 and a light chain comprising the amino acid sequence of SEQ ID NO: 202; or
x. a heavy chain comprising the amino acid sequence of SEQ ID NO: 236 and a light chain comprising the amino acid sequence of SEQ ID NO: 206.

43. The CAR therapy for use or the method of any of claims 35-42, wherein the PD-1 inhibitor comprises an anti-PD-1 antibody molecule comprising a heavy chain variable domain comprising the amino acid sequence of SEQ ID NO: 172 and a light chain variable domain comprising the amino acid sequence of SEQ ID NO: 204.

44. The CAR therapy for use or the method of claim 43, wherein the anti-PD1 antibody molecule comprises:
(i) a heavy chain variable (VH) region comprising the VHCDR1 amino acid sequence of SEQ ID NO: 503; the VHCDR2 amino acid sequence of SEQ ID NO: 504; and the VHCDR3 amino acid sequence of SEQ ID NO: 505; and
(ii) a light chain variable (VL) region comprising the VLCDR1 amino acid sequence of SEQ ID NO: 500; the VLCDR2 amino acid sequence of SEQ ID NO: 501; and rge VLCDR3 amino acid sequence of SEQ ID NO: 502,
or an amino acid sequence at least 85%, 90%, 95% identical or higher.

45. The CAR therapy for use or the method of any of the preceding claims, wherein the CAR comprises an anti-EGFRvIII binding domain, a transmembrane domain, and an intracellular signaling domain comprising a stimulatory domain, and wherein said anti-EGFRvIII binding domain comprises one or more of light chain complementary determining region 1 (LC CDR1), light chain complementary determining region 2 (LC CDR2), and light chain complementary determining region 3 (LC CDR3) of any anti-EGFRvIII light chain binding domain amino acid sequence listed in Table 2 or SEQ ID NO:11, and one or more of heavy chain complementary determining region 1 (HC CDR1), heavy chain complementary determining region 2 (HC CDR2), and heavy chain complementary determining region 3 (HC CDR3) of any anti-EGFRvIII heavy chain binding domain amino acid sequence listed in Table 2 or SEQ ID NO:11.

46. The CAR therapy for use or the method of any of the preceding claims, wherein the anti-EGFRvIII binding domain comprises a LC CDR1, LC CDR2 and LC CDR3 of any anti-EGFRvIII light chain binding domain amino acid sequence listed in Table 2 or SEQ ID NO:11.

47. The CAR therapy for use or the method of any of the preceding claims, wherein the anti-EGFRvIII binding domain comprises an LC CDR1, LC CDR2 and LC CDR3 of any anti-EGFRvIII light chain binding domain amino acid sequence listed in Table 2 or SEQ ID NO:11 and/or an HC CDR1, HC CDR2, and HC CDR3 of any anti-EGFRvIII heavy chain binding domain amino acid sequence listed in Table 2 or SEQ ID NO:11.

48. The CAR therapy for use or the method of any of the preceding claims, wherein the anti-EGFRvIII binding domain comprises any light chain variable region listed in Table 2 or SEQ ID NO:11 and/or any heavy chain variable region listed in Table 2 or SEQ ID NO:11.

49. The CAR therapy for use or the method of any of the preceding claims, wherein the anti-EGFRvIII binding domain is an scFv.

50. The CAR therapy for use or the method of any of the preceding claims, wherein the anti-EGFRvIII binding domain is comprises a light chain variable region comprising an amino acid sequence having at least one, two, or three modifications but not more than 30, 20, or 10 modifications of an amino acid sequence of a light chain variable region provided in Table 2 or SEQ ID NO:11, or a sequence with 95-99% identity with an amino acid sequence provided in Table 2 or SEQ ID NO:11 and/or a heavy chain variable region comprising an amino acid sequence having at least one, two or three modifications but not more than 30, 20 or 10 modifications of an amino acid sequence of a heavy chain variable region provided in Table 2 or SEQ ID NO:11, or a sequence with 95-99% identity to an amino acid sequence provided in Table 2 or SEQ ID NO:11.

51. The CAR therapy for use or the method of any of the preceding claims, wherein the anti-EGFRvIII binding domain comprises a sequence selected from a group consisting of SEQ ID NO:38, SEQ ID NO:44, SEQ ID NO:50, SEQ ID NO:56, SEQ ID NO:62, SEQ ID NO:68, SEQ ID NO:74, SEQ ID NO:80, and SEQ ID NO:86, or a sequence with 95-99% identify thereof.

52. The CAR therapy for use or the method of any of the preceding claims wherein the transmembrane domain comprises a transmembrane domain from a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 and CD154.

53. The CAR therapy for use or the method of any of the preceding claims, wherein the transmembrane domain comprises
(i) the amino acid sequence of SEQ ID NO: 15,
(ii) an amino acid sequence comprises at least one, two or three modifications but not more than 20, 10 or 5 modifications of the amino acid sequence of SEQ ID NO:15, or
(iii) a sequence at least 95% identical, e.g., with 95-99% identity, to the amino acid sequence of SEQ ID NO:15.

54. The CAR therapy for use or the method of any of the preceding claims, wherein
the CD19 binding domain is connected to the transmembrane domain by a hinge region.

55. The CAR therapy for use or the method of any of the preceding claims, wherein the hinge region comprises SEQ ID NO:14, or a sequence at least 95% identical, e.g., with 95-99%, identity thereof.

56. The CAR therapy for use or the method of any of the preceding claims, wherein the intracellular signaling domain comprises a costimulatory signaling domain, optionally further comprising a functional signaling domain obtained from a protein selected from the group consisting of a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

57. The CAR therapy for use or the method of claim 56, wherein the costimulatory domain comprises the amino acid sequence of SEQ ID NO:16, or an amino acid sequence having at least one, two, or three modifications but not more than 20, 10, or 5 modifications of the amino acid sequence of SEQ ID NO:16 or an amino acid sequence at least 95% identical to the amino acid sequence of SEQ ID NO:16.

58. The CAR therapy for use or the method of any of the preceding claims, wherein the intracellular signaling domain comprises a functional signaling domain of 4-1BB and/or a functional signaling domain of CD3 zeta.

59. The CAR therapy for use or the method of any of the preceding claims, wherein the intracellular signaling domain comprises the amino acid sequence of SEQ ID NO: 16 and/or the amino acid sequence of SEQ ID NO:17 or SEQ ID NO: 99; or an amino acid sequence having at least one, two, or three modifications but not more than 20, 10, or 5 modifications of the amino acid sequence of SEQ ID NO:16 and/or the amino acid sequence of SEQ ID NO:17 or SEQ ID NO: 99; or an amino acid sequence at least 95% identical to the amino acid sequence of SEQ ID NO:16 and/or the amino acid sequence of SEQ ID NO:17 or SEQ ID NO:99.

60. The CAR therapy for use or the method of any of the preceding claims, wherein the intracellular signaling domain comprises the amino acid sequence of SEQ ID NO:16 and the amino acid sequence of SEQ ID NO:17 or SEQ ID NO:99, wherein the amino acid sequences comprising the intracellular signaling domain are expressed in the same frame and as a single polypeptide chain.

61. The CAR therapy for use or the method of any of the preceding claims, wherein the CAR further comprises a leader sequence, optionally further comprising the amino acid sequence of SEQ ID NO:13 or a sequence with 95-99% identity to an amino acid sequence of SEQ ID NO:13.

62. The CAR therapy for use or the method of any of the preceding claims, wherein the CAR comprises:
(i) the amino acid sequence of any of SEQ ID NO:38, 44, 50, 56, 62, 68, 74, or 80;
(ii) an amino acid sequence having at least one, two or three modifications but not more than 30, 20 or 10 modifications to any of SEQ ID NO:38, 44, 50, 56, 62, 68, 74, or 80; or
(iii) an amino acid sequence at least 95 identical to any of SEQ ID NO:38, 44, 50, 56, 62, 68, 74, or 80.

63. The CAR therapy for use or the method of any of the preceding claims, wherein the cell comprising a CAR comprises a nucleic acid encoding the CAR.

64. The CAR therapy for use or the method of claim 63, wherein the nucleic acid encoding the CAR is a vector.

65. The CAR therapy for use or the method of claim 63 or 64, wherein the nucleic acid encoding the CAR is introduced into the cells by transduction of a plasmid, a lentivirus vector, adenoviral vector, or a retrovirus vector.

66. The CAR therapy for use or the method of any of claim 65, wherein the nucleic acid encoding the CAR is an RNA, e.g., an in vitro transcribed RNA.

67. The CAR therapy for use or the method of claim 66, wherein the nucleic acid encoding the CAR is introduced into the cells by electroporation.

68. The CAR therapy for use or the method of any of the preceding claims, wherein the cell is a T cell or an NK cell.

69. The CAR therapy for use or the method of claim 68, wherein the T cell is an autologous or allogeneic T cell.

70. The CAR therapy for use or the method of any of the preceding claims, further comprising administering an additional anti-cancer agent.

71. The CAR therapy for use or the method of any of the preceding claims, wherein the cancer is glioblastoma.

72. The CAR therapy for use or the method of any of the preceding claims, wherein the cancer is MGMT-unmethylated glioblastoma.

73. The CAR therapy for use or the method of any of the preceding claims, wherein radiation is administered to the subject prior to the CAR therapy.

74. The CAR therapy for use or the method of claim 73, wherein the radiation total dose of radiation is about 40 Gy.

75. The CAR therapy for use or the method of claim 74, wherein the radiation is delivered in 15 fractions over three weeks.

76. The CAR therapy for use or the method of any of the preceding claims, wherein the subject is a mammal, e.g., a human.

77. The CAR therapy for use or the method of any of the preceding claims, wherein the subject is an adult.

78. The CAR therapy for use or the method of any of the preceding claims, wherein the subject expresses PD-1, PD-L1, and/or PD-L2.

79. The CAR therapy for use or the method of claim 78, wherein a cancer cell or a cell in close proximity to a cancer cell in the subject expresses PD-1, PD-L1, and/or PD-L2.

80. The CAR therapy for use or the method of any of the preceding claims, wherein the subject has, or is identified as having, a higher number or percentage of immune effector cells, e.g., CD4⁺ and/or CD8⁺ T cells, expressing one, two, three, or all of PD-1, LAG-3 or TIM-3, compared to a reference value, e.g., a complete responder to the CAR therapy.

81. The CAR therapy for use or the method of 80, wherein the subject has, or is identified as having, a higher number of: PD-1 expressing immune effector cells, e.g., CD4⁺ and/or CD8⁺ T cells; PD-1 and LAG-3-expressing immune effector cells, e.g., CD4⁺ and/or CD8⁺ T cells; PD-1 and TIM-3 expressing immune effector cells, e.g., CD4⁺ and/or CD8⁺ T cells; or PD-1, TIM-3 and LAG-3 expressing immune effector cells, e.g., CD4⁺ and/or CD8⁺ T cells.

82. The CAR therapy for use or the method of 80 or 81, wherein the immune effector cells, e.g., CD4⁺ and/or CD8⁺ T cells, coexpress a CAR, e.g., a EGFRvIII CAR.

83. A combination comprising:
a cell, e.g., a population of immune effector cells, comprising a CAR, wherein the CAR comprises an antigen binding domain, a transmembrane domain, and an intracellular signaling domain; and
a PD-1 inhibitor chosen pembrolizumab, nivolumab, or any of the antibody molecules from Table 6, e.g., comprising the variable light chain and the variable heavy chain amino acid sequences of SEQ ID NO: 204 and SEQ ID NO: 172,
for use in treating a cancer, in a subject.

84. A composition (e.g., one or more compositions or dosage forms), comprising:
a cell, e.g., a population of immune effector cells, comprising a CAR, wherein the CAR comprises an antigen binding domain, a transmembrane domain, and an intracellular signaling domain, and
a PD-1 inhibitor chosen from Table 6, e.g., comprising the variable light chain and the variable heavy chain amino acid sequences of SEQ ID NO: 204 and SEQ ID NO: 172.

85. The method, combination, or composition of any of the preceding claims, wherein the anti-EGFRvIII binding domain comprises SEQ ID NO: 68.

86. A method of treating MGMT-unmethylated glioblastoma in a subject in need thereof, comprising administering to the subject:
(i) low dose radiation; and
(ii) a combination comprising an effective amount of a CAR therapy comprising a population of immune effector cells expressing a chimeric antigen receptor (CAR), wherein the CAR comprises an antigen (e.g., a EGFRvIII) binding domain, a transmembrane domain, and an intracellular signaling domain; and an effective amount of a PD-1 inhibitor.

87. The method of claim 86, wherein the population of immune effector cells expressing a chimeric antigen receptor (CAR) comprises an EGFRvIII binding domain scFv encoded by SEQ ID NO: 68, optionally wherein the effective amount is between about 1.75×10⁸ and about 5×10⁸ cells.

88. The method of claim 86 or 87, wherein the PD-1 inhibitor is pembrolizumab, optionally wherein the effective amount is about 200 mg.

89. The method of any one of claims 86-88, wherein the low dose radiation of (i) is 40 Gy.

90. The method of claim 89, wherein the low dose radiation of (i) is administered in 15 fractions over three weeks.

91. The method of any one of claims 86-90, wherein the combination of (ii) is administered after the low dose radiation of (i).

92. The method of claim 91, wherein the combination of (ii) is administered at treatment intervals of three weeks.

93. The method of claim 91 or 92, wherein the combination of (ii) is administered three times.

94. The method of any one of claims 86-93, wherein the subject is taking an anti-epileptic drug.
